(19) United States
(12) Patent Application Publication
(10) Pub. No.: US 2025/0390019 A1
(43) Pub. Date: Dec. 25, 2025

(54) ONIUM SALT MONOMER, POLYMER, CHEMICALLY AMPLIFIED RESIST COMPOSITION, AND PATTERN FORMING METHOD

(71) Applicant: Shin-Etsu Chemical Co., Ltd., Tokyo (JP)

(72) Inventor: Masahiro Fukushima, Joetsu-shi (JP)

(73) Assignee: Shin-Etsu Chemical Co., Ltd., Tokyo (JP)

(21) Appl. No.: 19/234,681

(22) Filed: Jun. 11, 2025

(30) Foreign Application Priority Data
Jun. 20, 2024 (JP) 2024-099834

(51) Int. Cl. G03F 7/029; G03F 7/00

(52) U.S. Cl. CPC G03F 7/029; G03F 7/70025; G03F 7/70033

(57) ABSTRACT

Provided are: an onium salt monomer used in a chemically amplified resist composition that is excellent in solvent solubility and has high sensitivity and high contrast in photolithography using a high energy ray, is excellent in lithographic performance of EL, LWR, CDU, DOF, or the like, is resistant to pattern collapse even in fine pattern formation, and is excellent in etching resistance; a polymer containing a repeat unit derived from the onium salt monomer; a chemically amplified resist composition containing the polymer; and a pattern forming method using the chemically amplified resist composition.

An onium salt monomer having the following formula (a):

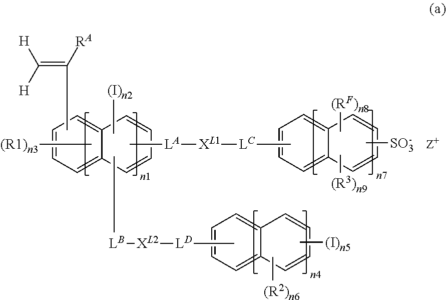

### CROSS-REFERENCE TO RELATED APPLICATION

This non-provisional application claims priority under 35 U.S.C. § 119 (a) on Patent Application No. 2024-099834 filed in Japan on Jun. 20, 2024, the entire contents of which are hereby incorporated by reference.

### TECHNICAL FIELD

The present invention relates to an onium salt monomer, a polymer, a chemically amplified resist composition, and a pattern forming method.

### BACKGROUND ART

With high integration and high speed of LSI, miniaturization of a pattern rule is rapidly progressing. In particular, expansion of a flash memory market and an increase in storage capacity lead miniaturization. As the most advanced miniaturization technique, mass production of a 65 nm node device by ArF lithography is performed, and preparation for mass production of a 45 nm node device by next-generation ArF immersion lithography is in progress. For a next-generation 32 nm node device, immersion lithography using an ultra-high NA lens in which a liquid having a refractive index higher than water, a high refractive index lens, and a high refractive index resist film are combined, extreme ultraviolet (EUV) lithography of a wavelength of 13.5 nm, double exposure (double patterning lithography) of ArF lithography, and the like are candidates, and studies are being conducted.

As miniaturization progresses and a diffraction limit of light is approached, contrast of light decreases. Due to a decrease in contrast of light, resolution of a hole pattern or a trench pattern and a focus margin decrease in a positive resist film.

Line width roughness (LWR) of a line pattern and dimensional uniformity (CDU) of a hole pattern have been regarded as problems along with miniaturization of a pattern. An influence of uneven distribution and aggregation of a base polymer and an acid generator and an influence of acid diffusion have been pointed out. Furthermore, LWR tends to increase as a resist film is thinned, and deterioration of LWR due to thinning with progress of miniaturization has become a serious problem.

In a resist composition for EUV lithography, it is necessary to simultaneously achieve high sensitivity, high resolution, and low LWR. If an acid diffusion distance is shortened, LWR is decreased but sensitivity is lowered. For example, by lowering a post exposure bake (PEB) temperature, LWR is decreased but sensitivity is lowered. By increasing the addition amount of a quencher, LWR is decreased but sensitivity is lowered. It is necessary to break a trade-off relationship between sensitivity and LWR.

In order to suppress acid diffusion, a resist compound containing a repeat unit derived from a sulfonic acid onium salt having a polymerizable unsaturated bond has been proposed (Patent Documents 1 and 2). Such a so-called polymer-bound acid generator is characterized by very short acid diffusion because a polymeric sulfonic acid is generated by exposure. In addition, sensitivity can also be improved by increasing a ratio of the acid generator. If the addition amount of an addition-type acid generator is increased, sensitivity is increased, but in this case, an acid diffusion distance is also increased. Since an acid diffuses non-uniformly, LWR and CDU deteriorate as acid diffusion increases. It can be said that the polymeric acid generator has high ability in a balance of sensitivity, LWR, and CDU.

Since an iodine atom has very large absorption of EUV of a wavelength of 13.5 nm, an effect of generating secondary electrons from the iodine atom during exposure has been confirmed, and the iodine atom has attracted attention in EUV lithography. Patent Document 3 describes a photoacid generator in which an iodine atom is introduced into an anion, and Patent Document 4 describes a polymerizable group-containing photoacid generator in which an iodine atom is introduced into an anion. Patent Document 5 describes a photoacid generator in which an iodine atom is introduced into each of a cation and an anion. As a result, although improvement in lithographic performance to some extent has been confirmed, an iodine atom does not have high organic solvent solubility, and there is a concern about deposition thereof in a solvent.

Patent Document 6 describes a photoacid generator in which a plurality of fluorine atoms are introduced into a cation. Although solvent solubility of the photoacid generator is improved by introduction of a plurality of fluorine atoms, it is not sufficient from a viewpoint of absorption of EUV, and there is room for improvement.

Patent Documents 7 to 11 describe a photoacid generator and a quencher (acid diffusion controlling agent) in which an iodine atom and a fluorine atom are contained in a cation. In addition, Patent Documents 12 to 15 describe an onium salt monomer in which an iodine atom and a polymerizable group are introduced into a cation. Furthermore, Patent Documents 16 and 17 describe an onium salt monomer in which an iodine atom and a polymerizable group are introduced into an anion. Although improvement in performance as a resist material has been confirmed by these developments, it is not yet satisfactory from a viewpoint of acid diffusion control, and further development of a resist material useful for fine pattern formation is required.

### CITATION LIST

Patent Document 1: JP 4425776
Patent Document 2: WO 2023/063203
Patent Document 3: JP 6720926
Patent Document 4: JP 6973274
Patent Document 5: JP 7041204
Patent Document 6: JP 7389562
Patent Document 7: JP-A 2021-123579
Patent Document 8: JP-A 2021-123580
Patent Document 9: JP-A 2022-123839
Patent Document 10: JP-A 2023-88869
Patent Document 11: JP-A 2023-88870
Patent Document 12: JP-A 2022-28615
Patent Document 13: JP-A 2023-93372
Patent Document 14: JP-A 2023-165660
Patent Document 15: JP-A 2023-171323
Patent Document 16: JP 6973274
Patent Document 17: WO 2024/014462

### SUMMARY OF THE INVENTION

In a chemically amplified resist composition using an acid as a catalyst, it is desired to develop a resist composition which has higher sensitivity, can improve lithographic performance such as exposure latitude (EL), LWR, CDU, or depth of focus (DOF), and is also excellent in etching resistance after pattern formation.

The present invention has been made in view of the above circumstances, and an object thereof is to provide: an onium salt monomer used in a chemically amplified resist composition that is excellent in solvent solubility, has high sensitivity and high contrast, is excellent in lithographic performance such as EL, LWR, CDU, or DOF, is resistant to pattern collapse even in fine pattern formation, and is excellent in etching resistance in photolithography using a high energy ray such as KrF excimer laser light, ArF excimer laser light, an electron beam (EB), or EUV; a polymer containing a repeat unit derived from the onium salt monomer; a chemically amplified resist composition containing the polymer; and a pattern forming method using the chemically amplified resist composition.

As a result of intensive studies to achieve the above object, the present inventors have found that a polymer containing a repeat unit derived from an onium salt monomer having an aromatic ring substituted with a polymerizable group and an iodine atom and having a structure in which a substituent including a fluorosulfonic acid anion structure and a substituent including an aromatic ring substituted with an iodine atom are bonded to the aromatic ring has good solvent solubility, and by using the polymer as a polymer-bound photoacid generator, a chemically amplified resist composition having high sensitivity, high contrast, high resolution, improved lithographic performance such as LWR or CDU, and excellent etching resistance after pattern formation can be obtained, thereby completing the present invention.

That is, the present invention provides the following onium salt monomer, polymer, chemically amplified resist composition, and pattern forming method.

1. An onium salt monomer having the following formula (a):

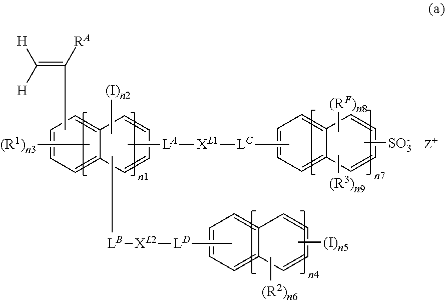

wherein n1 is 0 or 1, n2 is 1, 2, 3, or 4, n3 is 0, 1, or 2, provided that 1≤n2+n3≤4 when n1 is 0, and 1≤n2+n3≤6 when n1 is 1, n4 is 0 or 1, n5 is 1, 2, 3, or 4, n6 is 0, 1, or 2, provided that 1≤n5+n6≤4 when n4 is 0, and 1≤n5+n6≤6 when n4 is 1, n7 is 0 or 1, n8 is 0, 1, 2, 3, or 4, n9 is 0, 1, or 2, provided that 0≤n8+n9≤4 when n7 is 0, and 0≤n8+n9≤6 when n7 is 1,
R^{A} is a hydrogen atom, a fluorine atom, a methyl group, or a trifluoromethyl group,
R¹ is a halogen atom other than an iodine atom, a nitro group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when n3 is 2, R's may be the same as or different from each other, and two R¹s may be bonded to each other to form a ring together with carbon atoms to which they are bonded,
R² is a halogen atom other than an iodine atom, a nitro group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when n6 is 2, R²s may be the same as or different from each other, and two R²s may be bonded to each other to form a ring together with carbon atoms to which they are bonded,
R³ is a halogen atom other than a fluorine atom, a nitro group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when n9 is 2, R³s may be the same as or different from each other, and two R³s may be bonded to each other to form a ring together with carbon atoms to which they are bonded,
R^{F} is a fluorine atom, a C₁-C₆ fluorinated saturated hydrocarbyl group, a C₁-C₆ fluorinated saturated hydrocarbyloxy group, or a C₁-C₆ fluorinated saturated hydrocarbylthio group, when n8 is 2, 3, or 4, RFs may be the same as or different from each other,
L^{A}, L^{B}, L^{C}, and L^{D} are each independently a single bond, an ether bond, an ester bond, a sulfonic acid ester bond, an amide bond, a sulfonic acid amide bond, a carbonate bond, or a carbamate bond,
X^{L1} and X^{L2} are each independently a single bond or a C₁-C₄₀ hydrocarbylene group which may contain a heteroatom, and
Z⁺ is an onium cation.

2. The onium salt monomer according to the item 1, which has the following formula (a1):

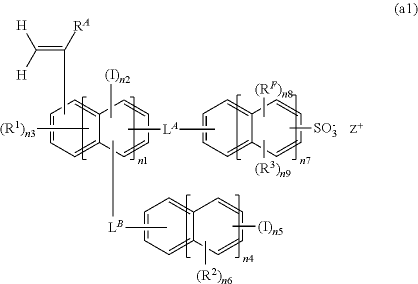

wherein n1 to n9, R^{A}, R¹, R², R³, R^{F}, L^{A}, L^{B}, and Z⁺ are as defined above.

3. The onium salt monomer according to the item 2, which has the following formula (a2):

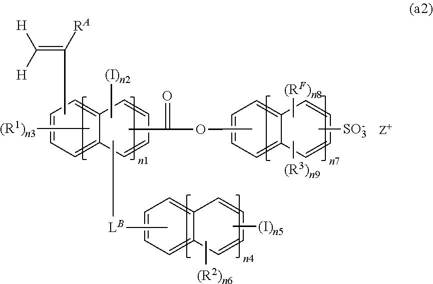

wherein n1 to n9, R^{A}, R¹, R², R³, R^{F}, L^{B}, and Z⁺ are as defined above.

4. The onium salt monomer according to any one of the items 1 to 3, wherein Z⁺ is a sulfonium cation having the following formula (Z-1) or an iodonium cation having the following formula (Z-2):

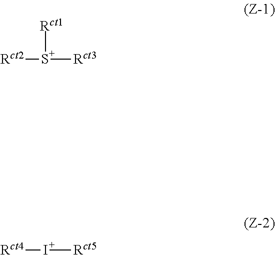

wherein R^{ct1} to R^{ct5} are each independently a halogen atom or a C₁-C₃₀ hydrocarbyl group which may contain a heteroatom, and R^{ct1} and R^{ct2} may be bonded to each other to form a ring together with a sulfur atom to which they are bonded.

5. The onium salt monomer according to any one of the items 1 to 3, wherein Z⁺ is a sulfonium cation having the following formula (Z-3):

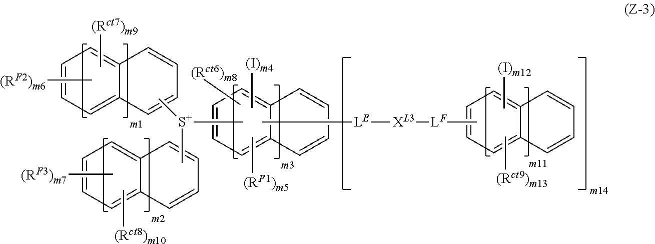

wherein m1 is 0 or 1, m2 is 0 or 1, m3 is 0 or 1, m4 is 0, 1, 2, 3, or 4, m5 is 0, 1, 2, 3, or 4, m6 is 0, 1, 2, 3, 4, 5, or 6, m7 is 0, 1, 2, 3, 4, 5, or 6, m8 is 0, 1, or 2, m9 is 0, 1, or 2, m10 is 0, 1, or 2, m11 is 0 or 1, m12 is 0, 1, 2, 3, or 4, m13 is 0, 1, or 2, m14 is 0, 1, or 2, provided that 0≤m6+m9≤4 when m1 is 0, and 0≤m6+m9≤6 when m1 is 1, 0≤m7+m10≤4 when m2 is 0, and 0≤m7+m10≤6 when m2 is 1, 1≤m4+m5+m8+m14≤4 when m3 is 0, and 1≤m4+m5+m8+m14≤6 when m3 is 1, 0≤m12+m13≤4 when m11 is 0, and 0≤m12+m13≤6 when m11 is 1, m4+m12≥1,
R^{F1} to R^{F3} are each independently a fluorine atom, a C₁-C₆ fluorinated saturated hydrocarbyl group, a C₁-C₆ fluorinated saturated hydrocarbyloxy group, or a C₁-C₆ fluorinated saturated hydrocarbylthio group, when m5 is 2 or more, R^{F1}s may be the same as or different from each other, when m6 is 2 or more, R^{F2}s may be the same as or different from each other, when m7 is 2 or more, R^{F3}s may be the same as or different from each other,
each of R^{ct6} to R^{ct9} is a halogen atom other than an iodine atom and a fluorine atom, a nitro group, a cyano group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when m8 is 2, two R^{ct6}s may be the same or different from each other, and may be bonded to each other to form a ring together with carbon atoms to which they are bonded, when m9 is 2, two R^{ct7}s may be the same or different from each other, and may be bonded to each other to form a ring together with carbon atoms to which they are bonded, when m10 is 2, two R^{ct8}s may be the same or different from each other, and may be bonded to each other to form a ring together with carbon atoms to which they are bonded, when m13 is 2, two R^{ct9}s may be the same or different from each other, and may be bonded to each other to form a ring with carbon atoms to which they are bonded,
aromatic rings directly bonded to S⁺ in the sulfonium cation may be bonded to each other to form a ring together with S⁺,
L^{E} and L^{F} are each independently a single bond, an ether bond, an ester bond, an amide bond, a sulfonic acid ester bond, a sulfonic acid amide bond, a carbonate bond, or a carbamate bond, and
X^{L3} is a single bond or a C₁-C₄₀ hydrocarbylene group which may contain a heteroatom.

6. The onium salt monomer according to the item 5, wherein the sulfonium cation having formula (Z-3) has the following formula (Z-3-1):

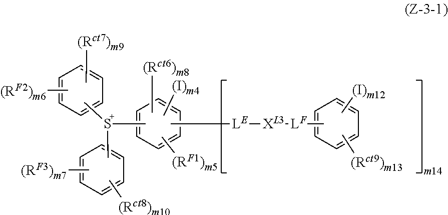

wherein m4 to m10, m12 to m14, R^{F1} to R^{F3}, R^{ct6} to R^{ct9}, L^{E}, L^{F}, and X^{L3} are as defined above.

7. The onium salt monomer according to the item 6, wherein the sulfonium cation having formula (Z-3-1) has the following formula (Z-3-2):

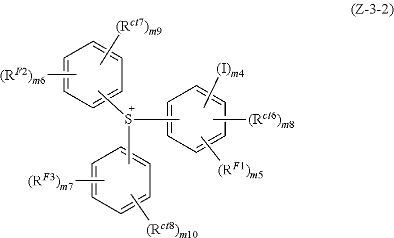

wherein m4 to m10, R^{F1} to R^{F3}, and R^{ct6} to R^{ct8} are as defined above.

8. A monomeric photoacid generator including the onium salt monomer according to any one of the items 1 to 7.

9. A polymer including a repeat unit derived from the monomeric photoacid generator according to the item 8.

10. The polymer according to the item 9, further including a repeat unit having the following formula (b1) or (b2):

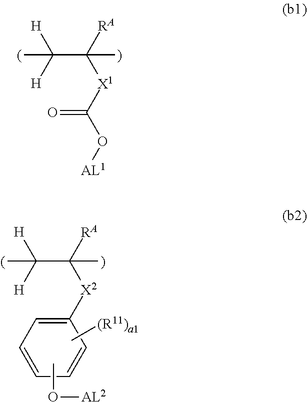

wherein R^{A}s are each independently a hydrogen atom, a fluorine atom, a methyl group, or a trifluoromethyl group,
X¹ is a single bond, a phenylene group, a naphthylene group, *—C(═O)—O—X¹¹—, or *—C(═O)—NH—X¹¹—, and the phenylene group or the naphthylene group may be substituted with a hydroxy group, a nitro group, a cyano group, a C₁-C₁₀ saturated hydrocarbyl group which may contain a fluorine atom, a C₁-C₁₀ saturated hydrocarbyloxy group which may contain a fluorine atom, or a halogen atom, X¹¹ is a C₁-C₁₀ saturated hydrocarbylene group, a phenylene group, or a naphthylene group, and the saturated hydrocarbylene group may contain a hydroxy group, an ether bond, an ester bond, or a lactone ring,
X² is a single bond, *—C(═O)—O—, or *—C(═O)—NH—,
* designates a point of attachment to the carbon atom of the main chain,
R¹¹ is a halogen atom, a cyano group, a hydroxy group, a nitro group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom,
AL¹ and AL² are each independently an acid-unstable group, and
a1 is 0, 1, 2, 3, or 4.

11. The polymer according to the item 9 or 10, further including a repeat unit having the following formula (b3):

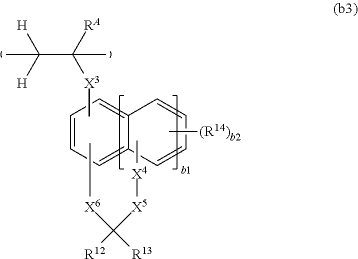

wherein b1 is 0 or 1, b2 is 0, 1, 2, or 3 when b1 is 0, and b2 is 0, 1, 2, 3, 4, or 5 when b1 is 1,
R^{A} is a hydrogen atom, a fluorine atom, a methyl group, or a trifluoromethyl group,
X³ is a single bond, *—C(═O)—O—, or *—C(═O)—NH—, * designates a point of attachment to the carbon atom of the main chain,
R¹² and R¹³ are each independently a hydrogen atom or a C₁-C₂₀ hydrocarbyl group which may contain a heteroatom, R¹² and R¹³ may be bonded to each other to form a ring together with a carbon atom to which they are bonded,
R¹⁴ is a halogen atom, a hydroxy group, a cyano group, a nitro group, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{14A})(R^{14B}), R^{14A} and R^{14B} are each independently a hydrogen atom or a C₁-C₆ hydrocarbyl group, when b2 is 2 or more, a plurality of R¹⁴s may be bonded to each other to form a ring together with aromatic ring carbon atoms to which they are bonded,
X⁴ is a single bond, a C₁-C₄ aliphatic hydrocarbylene group, a carbonyl group, a sulfonyl group, or a group obtained by combining these groups, and
X⁵ and X⁶ are each independently an oxygen atom or a sulfur atom, provided that X⁴ and X⁶ are bonded to adjacent aromatic ring carbon atoms.

12. The polymer according to any one of the items 9 to 11, further including a repeat unit having the following formula (c):

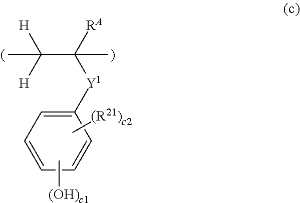

wherein R^{A} is a hydrogen atom, a fluorine atom, a methyl group, or a trifluoromethyl group,
Y¹ is a single bond, *—C(═O)—O—, or *—C(═O)—NH—, * designates a point of attachment to the carbon atom of the main chain,
R²¹ is a halogen atom, a nitro group, a cyano group, a carboxy group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom,
c1 is 1, 2, 3, or 4, and c2 is 0, 1, 2, or 3, provided that 1≤c1+c2≤5.

13. The polymer according to any one of the items 9 to 12, further including a repeat unit having the following formula (d):

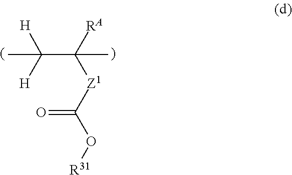

wherein R^{A} is a hydrogen atom, a fluorine atom, a methyl group, or a trifluoromethyl group,
Z¹ is a single bond, a phenylene group, a naphthylene group, *—C(═O)—O—Z¹¹—, or *—C(═O)—NH—Z¹¹—, and the phenylene group or the naphthylene group may be substituted with a hydroxy group, a nitro group, a cyano group, a C₁-C₁₀ saturated hydrocarbyl group which may contain a fluorine atom, a C₁-C₁₀ saturated hydrocarbyloxy group which may contain a fluorine atom, or a halogen atom, * designates a point of attachment to the carbon atom of the main chain, Z¹¹ is a C₁-C₁₀ saturated hydrocarbylene group, a phenylene group, or a naphthylene group, and the saturated hydrocarbylene group may contain a hydroxy group, an ether bond, an ester bond, or a lactone ring,
R³¹ is a hydrogen atom or a C₁-C₂₀ group containing at least one structure selected from a hydroxy group other than a phenolic hydroxy group, a cyano group, a carbonyl group, a carboxy group, an ether bond, an ester bond, a sulfonic acid ester bond, a carbonate bond, a lactone ring, a sultone ring, and a carboxylic anhydride (—C(═O)—O—C(═O)—).

14. A chemically amplified resist composition including a base polymer including (A) the polymer according to any one of the items 9 to 13.

15. The chemically amplified resist composition according to the item 14, further including (B) an organic solvent.

16. The chemically amplified resist composition according to the item 14 or 15, further including (C) a quencher.

17. The chemically amplified resist composition according to any one of the items 14 to 16, further including (D) an acid generator.

18. The chemically amplified resist composition according to any one of the items 14 to 17, further including (E) a surfactant.

19. The chemically amplified resist composition according to any of the items 14 to 18, further including (F) a dissolution inhibitor.

20. A pattern forming method including: a step of forming a resist film on a substrate using the chemically amplified resist composition according to any one of the items 14 to 19;

a step of exposing the resist film to a high energy ray; and a step of developing the exposed resist film using a developer.

21. The pattern forming method according to the item 20, wherein the high energy ray is ArF excimer laser light having a wavelength of 193 nm, KrF excimer laser light having a wavelength of 248 nm, EB, or EUV having a wavelength of 3 to 15 nm.

### Advantageous Effects of the Invention

A polymer containing a repeat unit that generates an acid upon exposure, the repeat unit being derived from an onium salt monomer having an aromatic ring substituted with a polymerizable group and an iodine atom and having a structure in which a substituent including a fluorosulfonic acid anion structure and a substituent including an aromatic ring substituted with an iodine atom are further bonded to the aromatic ring, has better solvent solubility due to a branched structure thereof. In EUV lithography of a wavelength of 13.5 nm, since an iodine atom has very large absorption of EUV, secondary electrons are generated from the iodine atom during exposure. Since the fluorosulfonic acid anion bonded to the aromatic ring substituted with a polymerizable group and an iodine atom has a short distance from a polymer main chain to an acid generation position, and is also at a position spatially close to the aromatic ring substituted with an iodine atom and bonded to the aromatic ring substituted with a polymerizable group and an iodine atom, secondary electrons generated from an iodine atom promotes decomposition of a cation located in the vicinity of the anion, and an acid is efficiently generated, whereby sensitivity is increased. Furthermore, since an iodine atom has a large atomic weight and a generated acid is bonded to the polymer main chain, acid diffusion is small. This makes it possible to prevent a decrease in resolution due to blurring of acid diffusion and to improve LWR and CDU. Furthermore, the aromatic ring in the repeat unit acts as a good etch-resistant group, and is suitable for fine pattern formation.

### DESCRIPTION OF THE PREFERRED EMBODIMENT

### Onium Salt Monomer

An onium salt monomer of the present invention has the following formula (a):

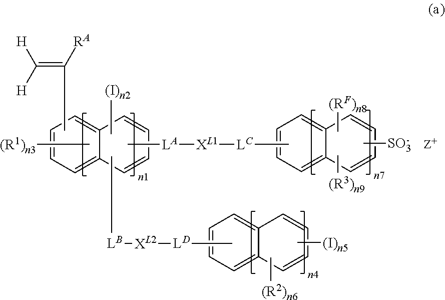

In formula (a), n1 is 0 or 1. When n1 is 0, it is a benzene ring, and when n1 is 1, it is a naphthalene ring, but a benzene ring in which n1 is 0 is preferable from a viewpoint of solvent solubility. n2 is 1, 2, 3, or 4. n2 is preferably 1, 2 or 3, more preferably 1 or 2, and still more preferably 1 from a viewpoint of raw material procurement. n3 is 0, 1, or 2. Provided that 1≤n2+n3≤4 when n1 is 0, and 1≤n2+n3≤6 when n1 is 1.

In formula (a), n4 is 0 or 1. When n4 is 0, it is a benzene ring, and when n4 is 1, it is a naphthalene ring, but a benzene ring in which n4 is 0 is preferable from a viewpoint of solvent solubility. n5 is 1, 2, 3, or 4, but preferably 1, 2, or 3. The larger the number of iodine atoms in the anion structure, the higher absorption particularly for EUV, but since there is a concern that solvent solubility is poor and precipitation occurs in a resist composition, the number of iodine atoms in the anion, that is, n2+n5 is preferably 2, 3, 4, or 5, and more preferably 2, 3, or 4. n6 is 0, 1, or 2. Provided that 1≤n5+n6≤4 when n4 is 0, and 1≤n5+n6≤6 when n4 is 1.

In formula (a), n7 is 0 or 1. When n7 is 0, it is a benzene ring, and when n7 is 1, it is a naphthalene ring, but a benzene ring in which n7 is 0 is preferable from a viewpoint of solvent solubility. n8 is 0, 1, 2, 3, or 4. n9 is 0, 1, or 2. Provided that 0≤n8+n9≤4 when n7 is 0, and 0≤n8+n9≤6 when n7 is 1.

In formula (a), R^{A} is a hydrogen atom, a fluorine atom, a methyl group, or a trifluoromethyl group. Among these, R^{A} is preferably a hydrogen atom or a methyl group, and more preferably a hydrogen atom.

In formula (a), R¹ is a halogen atom other than an iodine atom, a nitro group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. The halogen atom other than an iodine atom is preferably a fluorine atom, a chlorine atom, or a bromine atom, and more preferably a fluorine atom. The hydrocarbyl group and hydrocarbyl moieties of the hydrocarbyloxy group and the hydrocarbylthio group may be saturated or unsaturated, and may be linear, branched, or cyclic. Specific examples thereof include: a C₁-C₂₀ alkyl group such as a methyl group, an ethyl group, a n-propyl group, an isopropyl group, a n-butyl group, an isobutyl group, a sec-butyl group, a tert-butyl group, a n-pentyl group, a n-hexyl group, a n-octyl group, a n-nonyl group, a n-decyl group, an undecyl group, a dodecyl group, a tridecyl group, a tetradecyl group, a pentadecyl group, a heptadecyl group, an octadecyl group, a nonadecyl group, or an icosyl group; a C₃-C₂₀ cyclic saturated hydrocarbyl group such as a cyclopropyl group, a cyclopentyl group, a cyclohexyl group, a cyclopropylmethyl group, a 4-methylcyclohexyl group, a cyclohexylmethyl group, a norbornyl group, or an adamantyl group; a C₂-C₂₀ alkenyl group such as a vinyl group, an allyl group, a propenyl group, a butenyl group, or a hexenyl group; a C₃-C₂₀ cyclic unsaturated hydrocarbyl group such as a cyclohexenyl group; a C₆-C₂₀ aryl group such as a phenyl group or a naphthyl group; a C₇-C₂₀ aralkyl group such as a benzyl group, a 1-phenylethyl group, or a 2-phenylethyl group; and a group obtained by combining these. Some or all of hydrogen atoms of the hydrocarbyl group may be replaced with a group containing a heteroatom such as an oxygen atom, a sulfur atom, a nitrogen atom, or a halogen atom, some of (—CH₂—)s in the hydrocarbyl group may be replaced with a group containing a heteroatom such as an oxygen atom, a sulfur atom, or a nitrogen atom, and as a result, the hydrocarbyl group may contain a hydroxy group, a cyano group, a fluorine atom, a chlorine atom, a bromine atom, an iodine atom, a carbonyl group, an ether bond, an ester bond, a sulfonic acid ester bond, a carbonate bond, a lactone ring, a sultone ring, a carboxylic acid anhydride (—C(═O)—O—C(═O)—), a haloalkyl group, or the like. When n3 is 2, R¹s may be the same as or different from each other.

When n3 is 2, two R¹s may be bonded to each other to form a ring together with carbon atoms to which they are bonded. Specific examples of the ring formed at this time include a cyclopropane ring, a cyclobutane ring, a cyclopentane ring, a cyclohexane ring, a norbornane ring, and an adamantane ring. Some or all of hydrogen atoms of the ring may be replaced with a group containing a heteroatom such as an oxygen atom, a sulfur atom, a nitrogen atom, or a halogen atom, some of (—CH₂—)s in the ring may be replaced with a group containing a heteroatom such as an oxygen atom, a sulfur atom, or a nitrogen atom, and as a result, the ring may contain a hydroxy group, a fluorine atom, a chlorine atom, a bromine atom, an iodine atom, a cyano group, a carbonyl group, an ether bond, an ester bond, a sulfonic acid ester bond, a carbonate bond, a lactone ring, a sultone ring, a carboxylic acid anhydride (—C(═O)—O—C(═O)—), a haloalkyl group, or the like.

In formula (a), R² is a halogen atom other than an iodine atom, a nitro group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. Specific examples of the halogen atom other than an iodine atom include a fluorine atom, a chlorine atom, and a bromine atom. The hydrocarbyl group and hydrocarbyl moieties of the hydrocarbyloxy group and the hydrocarbylthio group may be saturated or unsaturated, and may be linear, branched, or cyclic. Specific examples thereof include, but are not limited to, those similar to those exemplified as the hydrocarbyl group of R¹. When n6 is 2, R²s may be the same as or different from each other.

When n6 is 2, two R²s may be bonded to each other to form a ring together with carbon atoms to which they are bonded. The ring is preferably a 5- to 8-membered ring.

In formula (a), R³ is a halogen atom other than a fluorine atom, a nitro group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a Specific examples of the halogen atom other than a fluorine atom include a heteroatom. chlorine atom, a bromine atom, and an iodine atom. The hydrocarbyl group and hydrocarbyl moieties of the hydrocarbyloxy group and the hydrocarbylthio group may be saturated or unsaturated, and may be linear, branched, or cyclic. Specific examples thereof include, but are not limited to, those similar to those exemplified as the hydrocarbyl group of R¹. When n9 is 2, R³s may be the same as or different from each other.

When n9 is 2, two R³s may be bonded to each other to form a ring together with carbon atoms to which they are bonded. The ring is preferably a 5- to 8-membered ring.

In formula (a), RFs are each independently a fluorine atom, a C₁-C₆ fluorinated saturated hydrocarbyl group, a C₁-C₆ fluorinated saturated hydrocarbyloxy group, or a C₁-C₆ fluorinated saturated hydrocarbylthio group. Among these, R^{F} is preferably a fluorine atom, a trifluoromethyl group, a trifluoromethoxy group, or a trifluoromethylthio group, and more preferably a fluorine atom. When n8 is 2, 3, or 4, R^{F}s may be the same as or different from each other.

In formula (a), L^{A}, L^{B}, L^{C}, and L^{D} are each independently a single bond, an ether bond, an ester bond, a sulfonic acid ester bond, an amide bond, a sulfonic acid amide bond, a carbonate bond, or a carbamate bond. Among these, L^{A} is preferably a single bond, an ether bond, an ester bond, or a sulfonic acid ester bond, and more preferably an ester bond or a sulfonic acid ester bond. L^{B} is preferably a single bond, an ether bond, an ester bond, or a sulfonic acid ester bond, and more preferably an ester bond or a sulfonic acid ester bond. L^{C} is preferably a single bond, an ether bond, an ester bond, or a sulfonic acid ester bond, and more preferably a single bond, an ether bond, or an ester bond. L^{D} is preferably a single bond, an ether bond, an ester bond, or a sulfonic acid ester bond, and more preferably a single bond, an ether bond, or an ester bond.

L^{A} and L^{B} are preferably bonded to adjacent aromatic ring carbon atoms. At this time, since the substituent including an aromatic sulfonic acid anion structure and the substituent including an aromatic ring substituted with an iodine atom are present at spatially closer positions, higher sensitivity is expected.

In formula (a), X^{L1} and X^{L2} are each independently a single bond or a C₁-C₄₀ hydrocarbylene group which may contain a heteroatom. The hydrocarbylene group may be linear, branched, or cyclic, and specific examples thereof include an alkanediyl group, a cyclic saturated hydrocarbylene group, and an arylene group. Specific examples of the heteroatom include an oxygen atom, a nitrogen atom, and a sulfur atom.

Specific examples of the C₁-C₄₀ hydrocarbylene group which may contain a heteroatom, represented by X^{L1} and X^{L2} include, but are not limited to, those listed below. Note that, in the following formulas, * designates a point of attachment to L^{A}, L^{C}, L^{B}, or L^{D}.

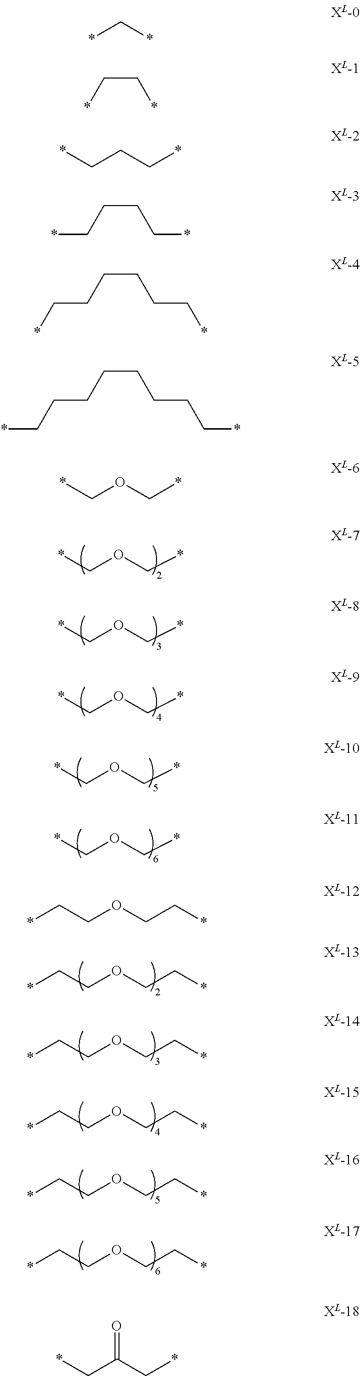

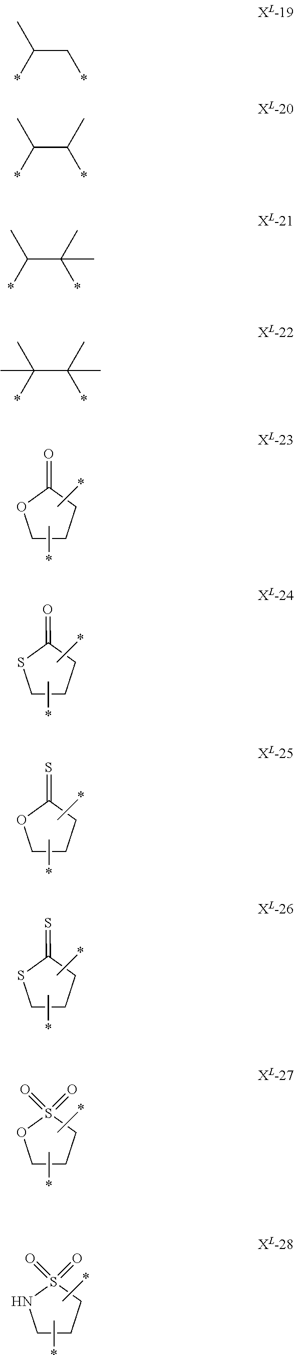

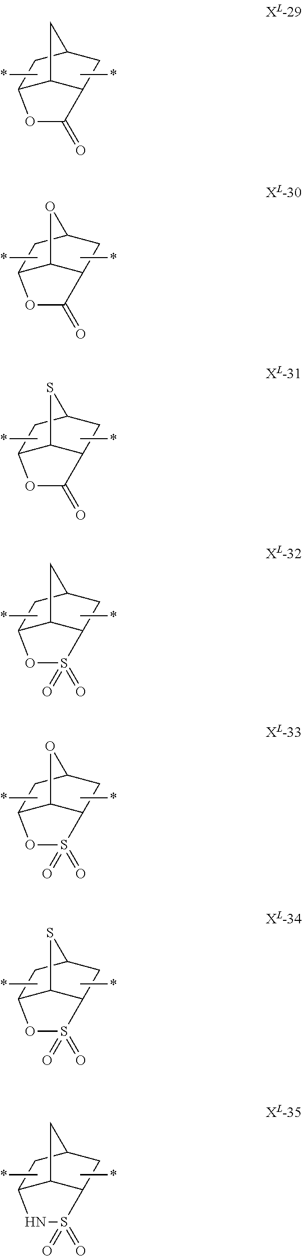

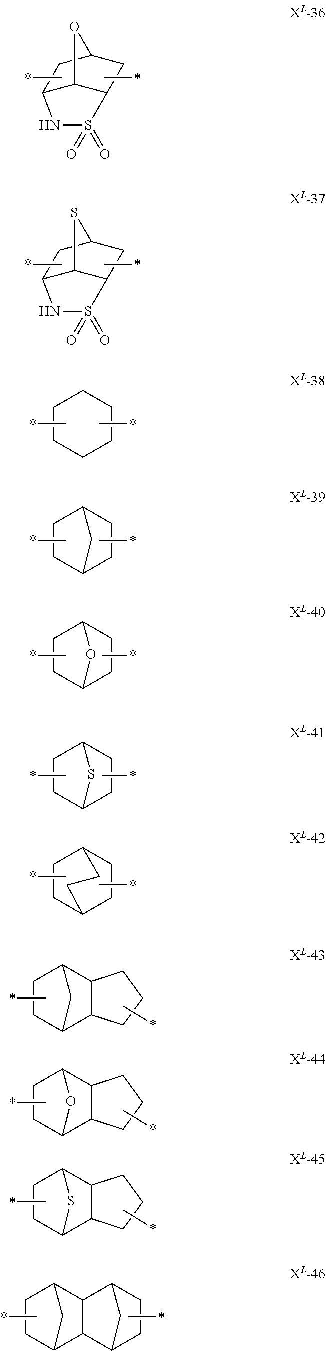

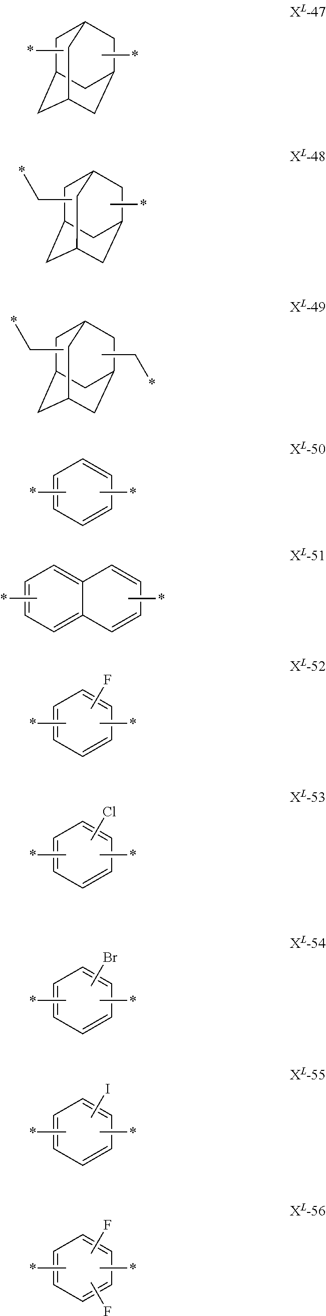

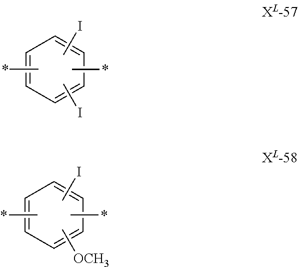

Among these, X^{L}-0 to X^{L}-22, X^{L}-29 to X^{L}-34, and X^{L}-47 to X^{L}-58 are preferable.

The onium salt monomer having formula (a) preferably has the following formula (a1):

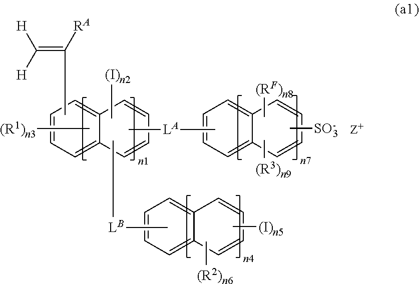

wherein n1 to n9, R^{A}, R¹, R², R³, R^{F}, L^{A}, L^{B}, and Z⁺ are as defined above.

The onium salt monomer having formula (a1) preferably has the following formula (a2):

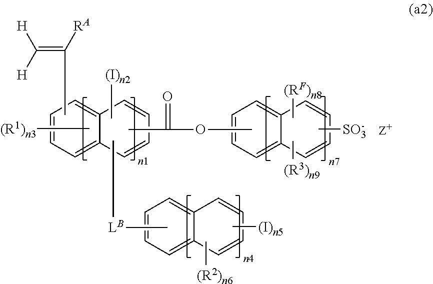

wherein n1 to n9, R^{A}, R¹, R², R³, R^{F}, L^{B}, and Z⁺ are as defined above.

Examples of an anion of the onium salt monomer having formula (a) include, but are not limited to, those listed below. Note that, in the following formulas, R^{A} is as defined above, and Me is a methyl group. Bonding positions of various substituents on an aromatic ring may be replaced with each other.

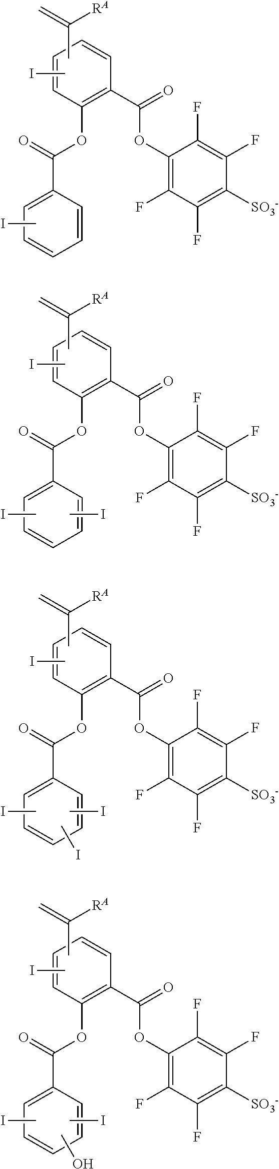

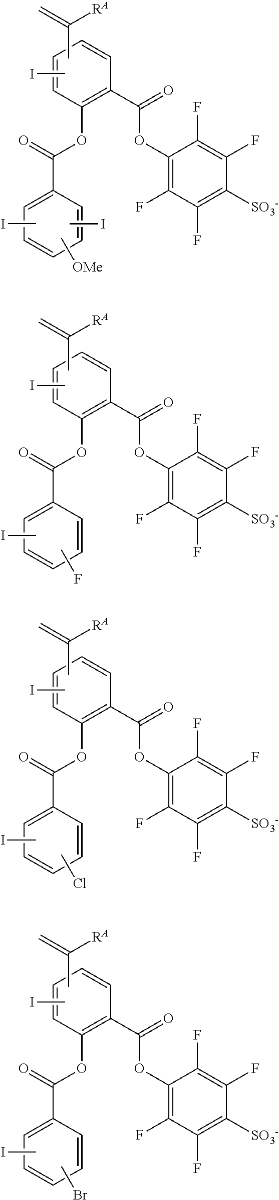

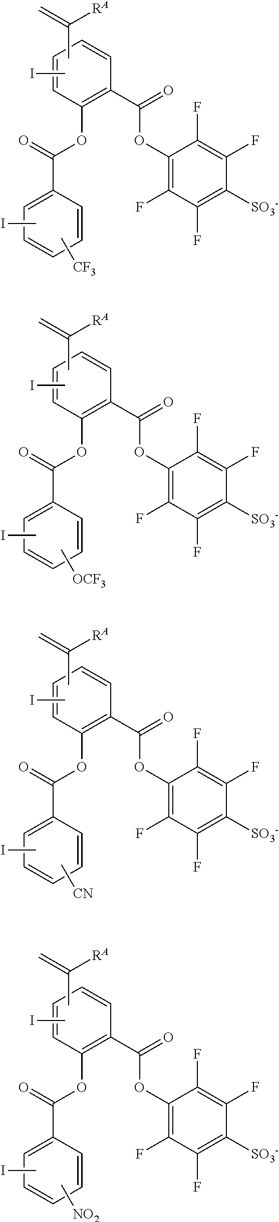

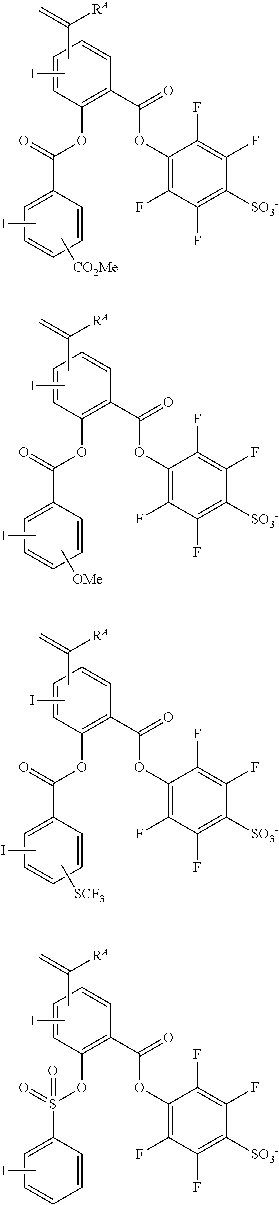

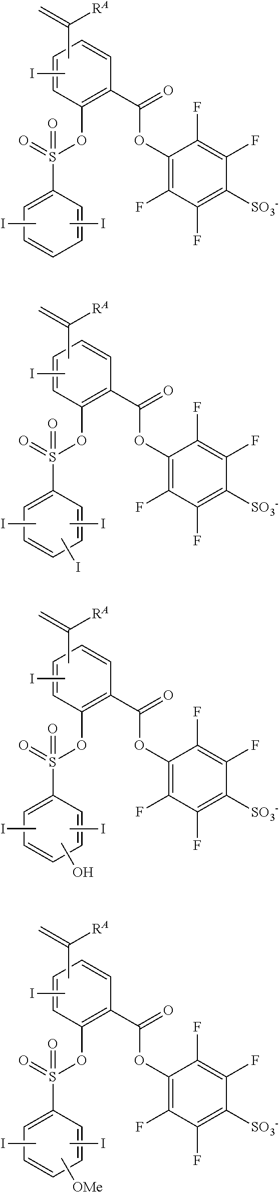

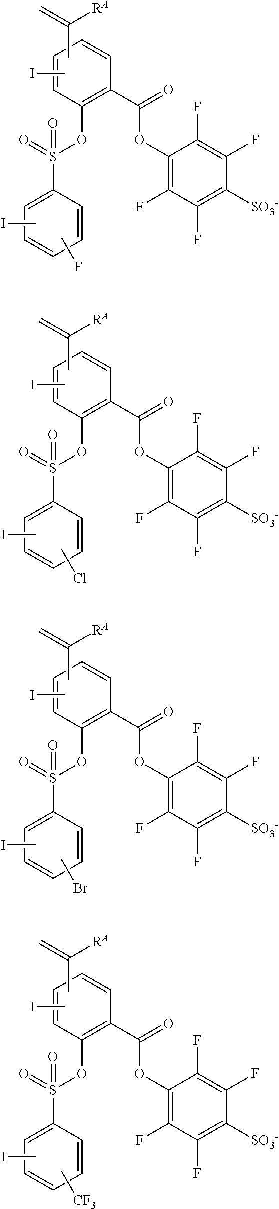

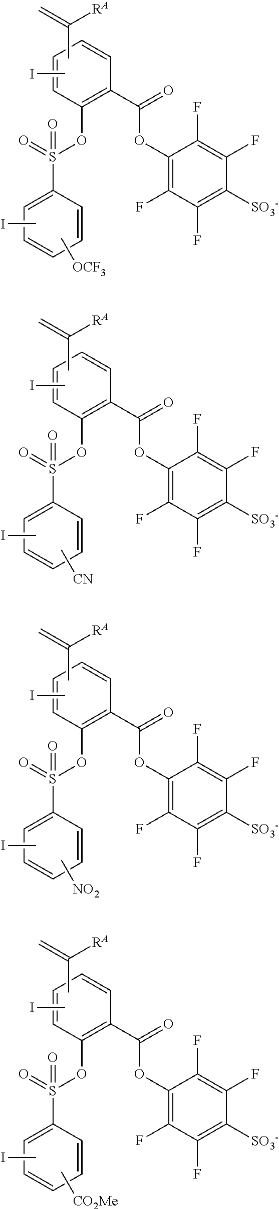

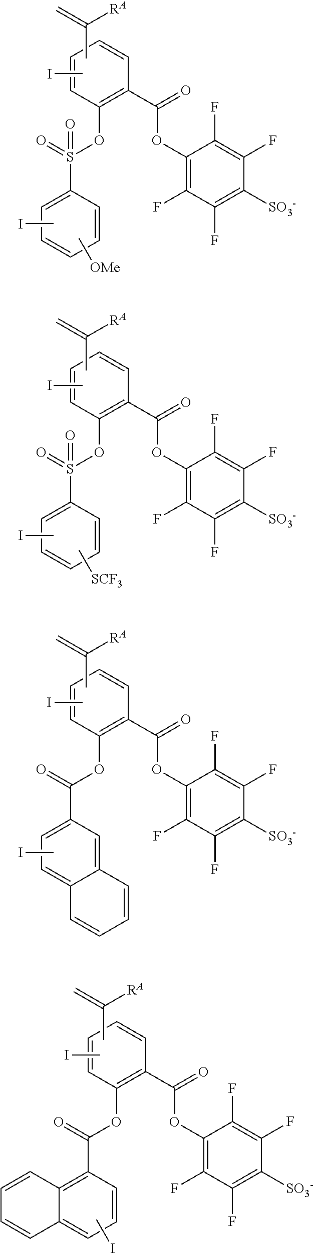

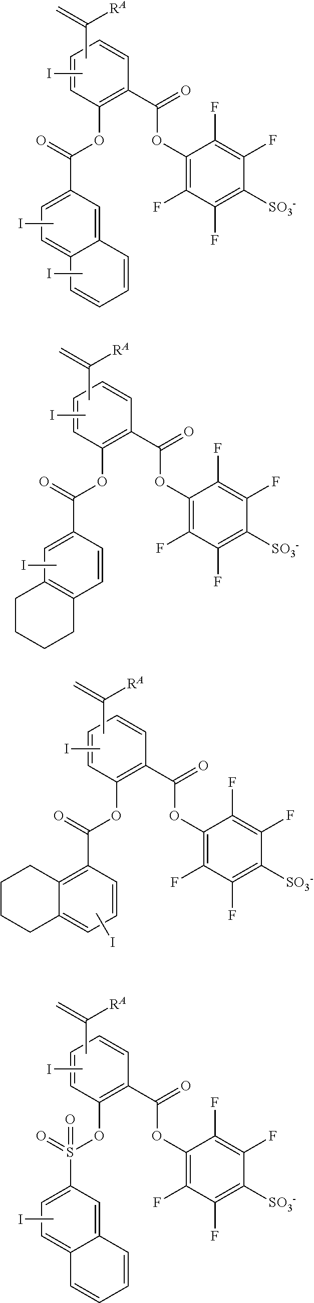

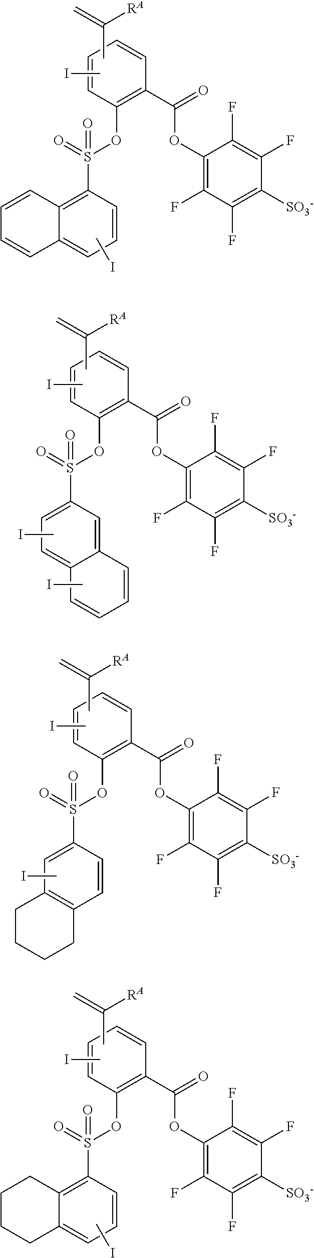

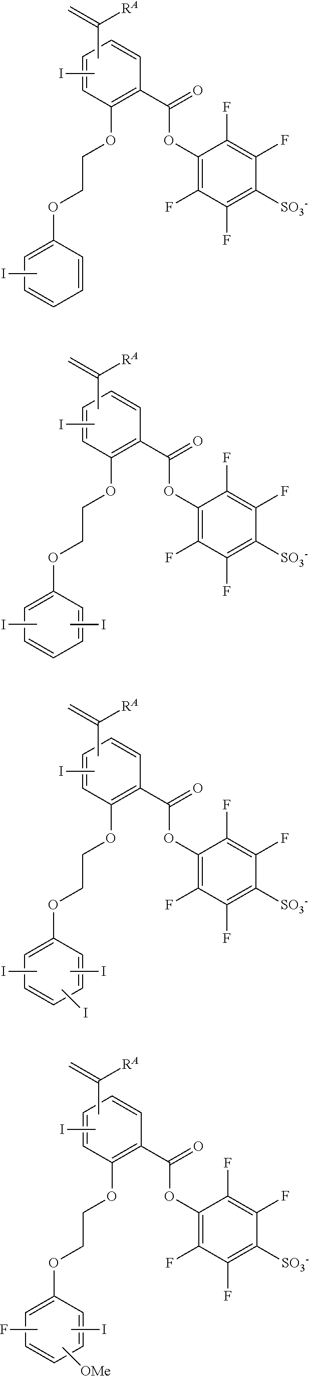

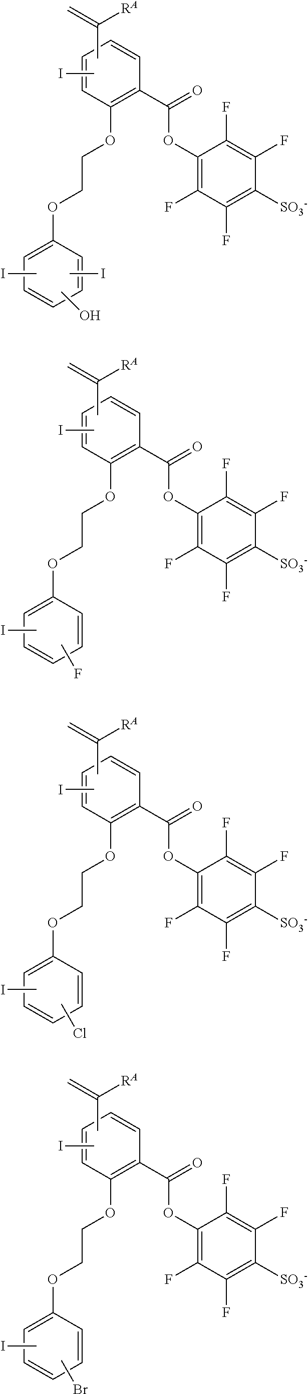

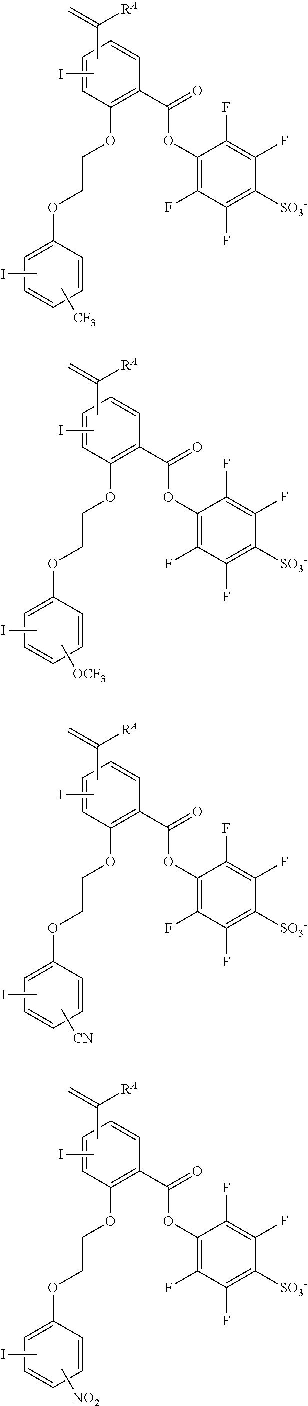

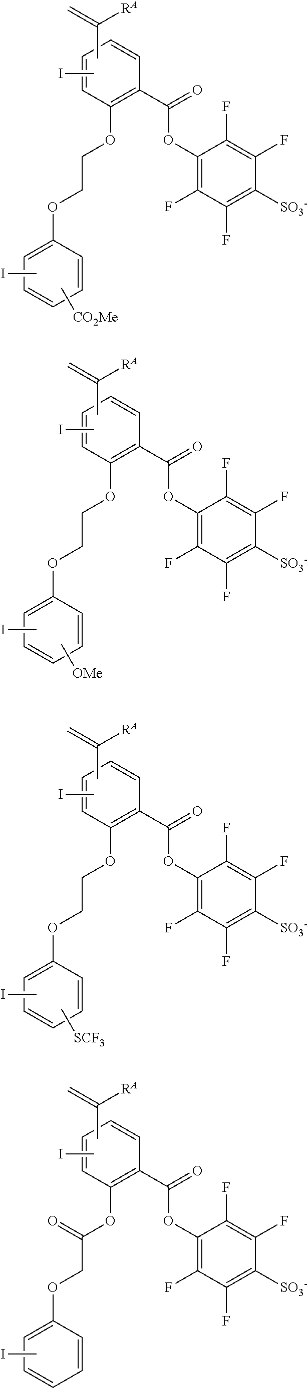

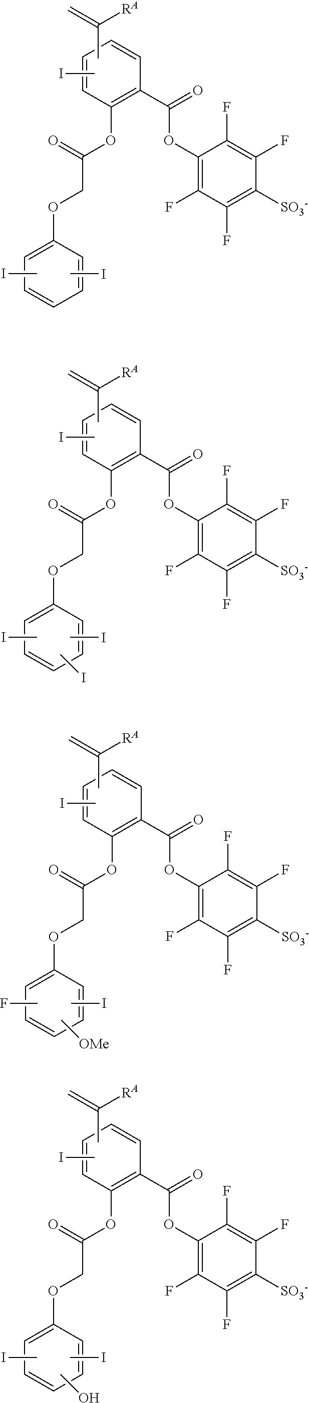

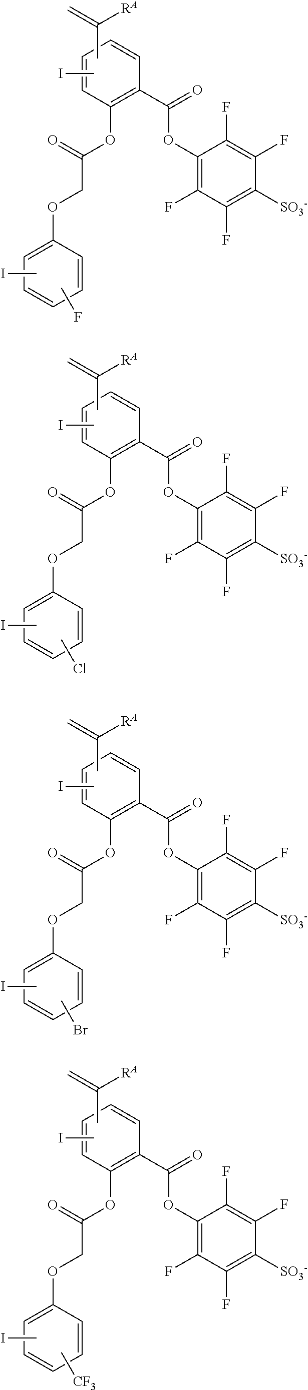

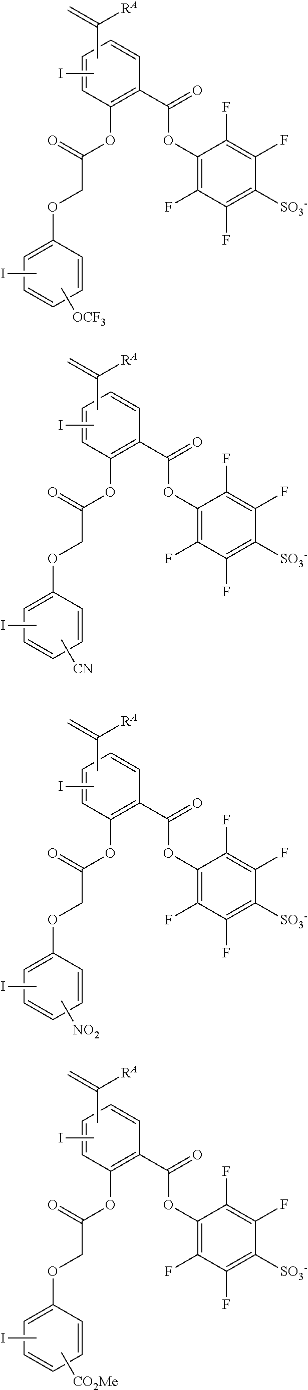

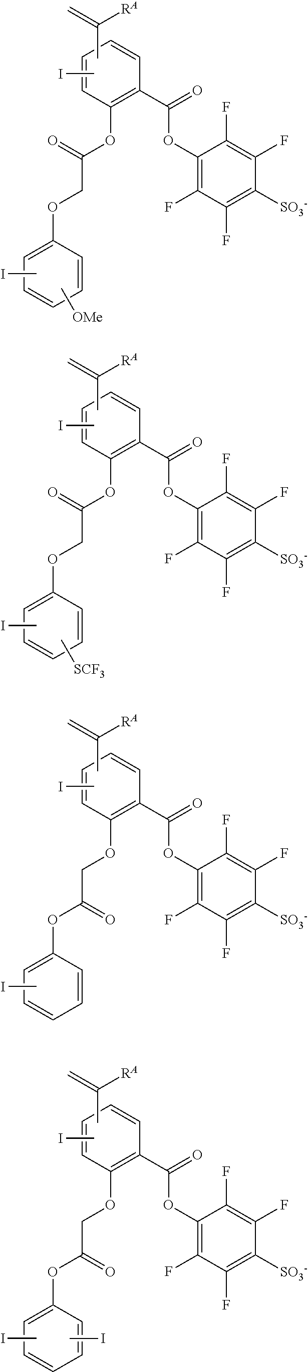

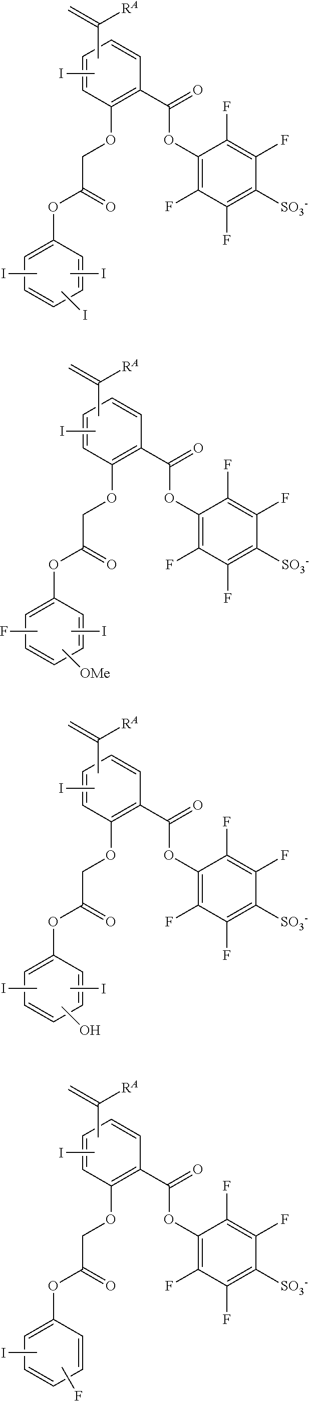

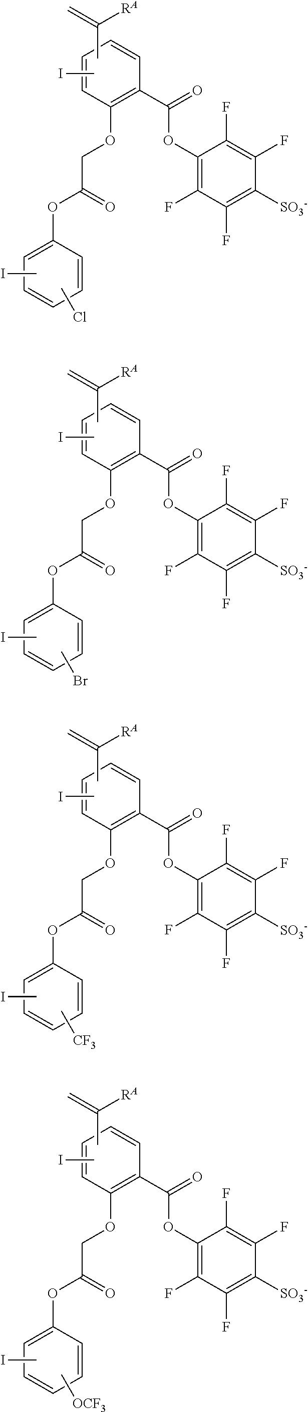

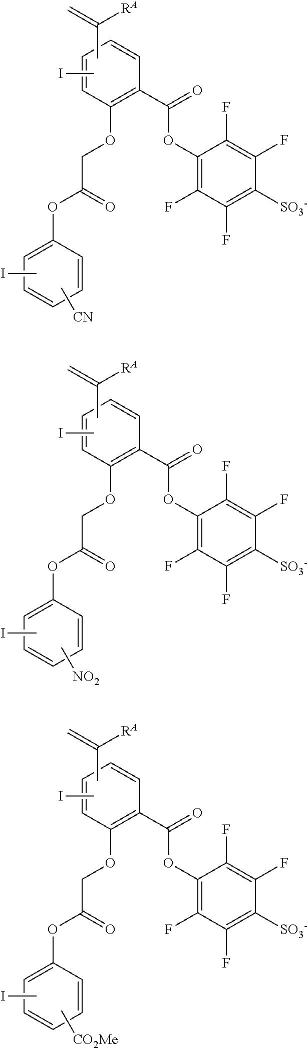

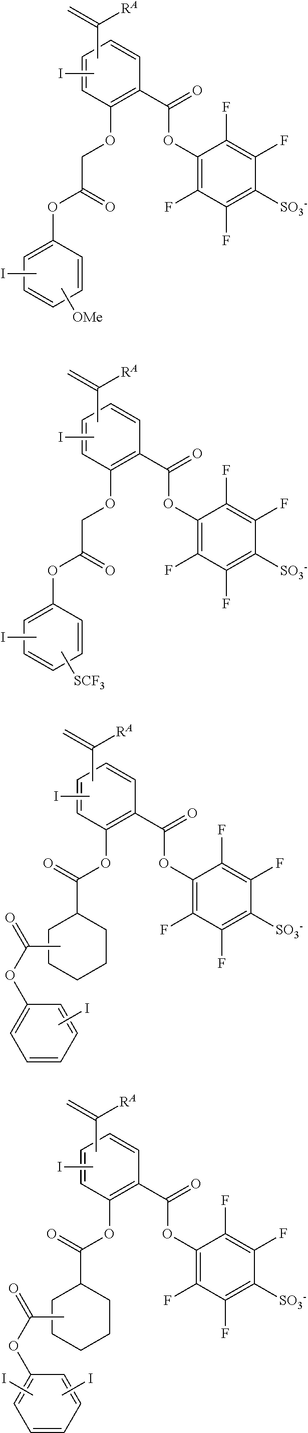

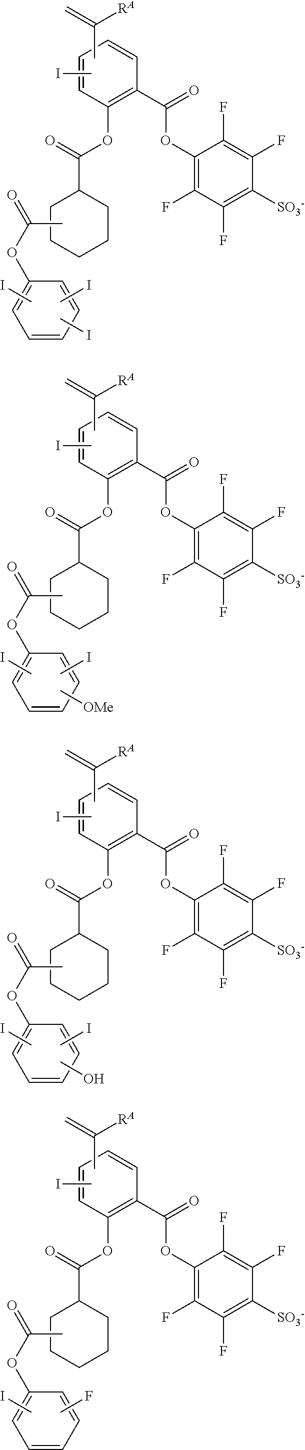

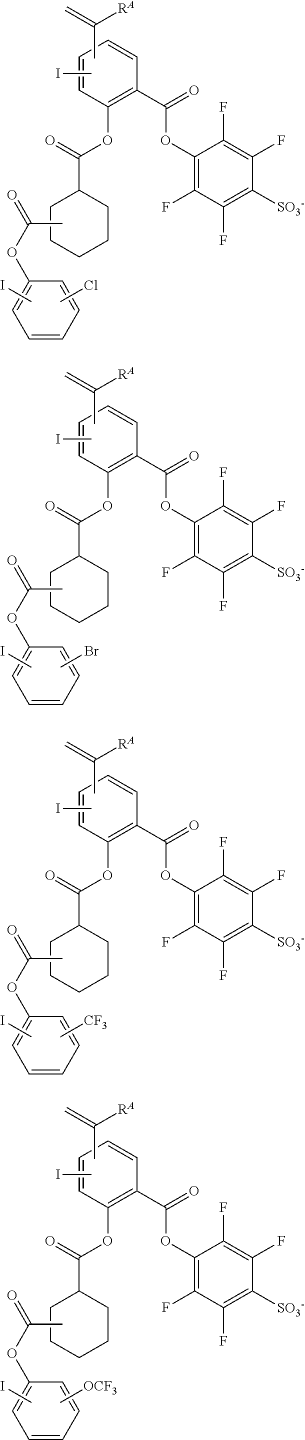

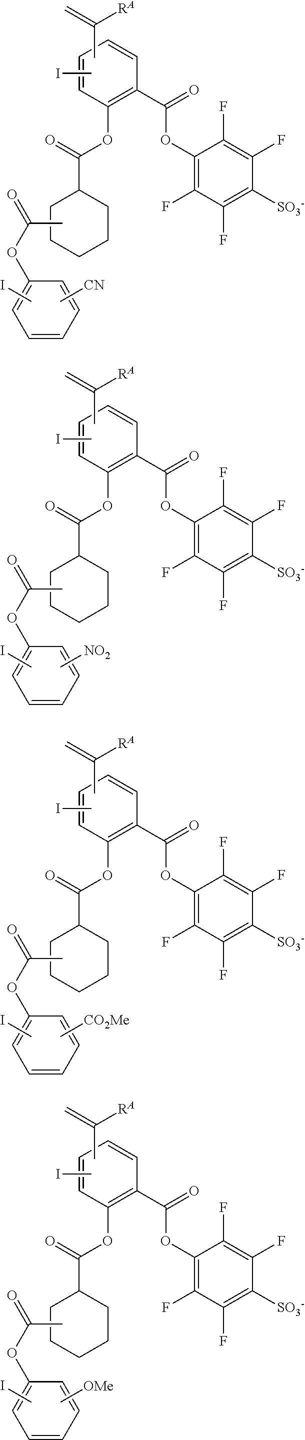

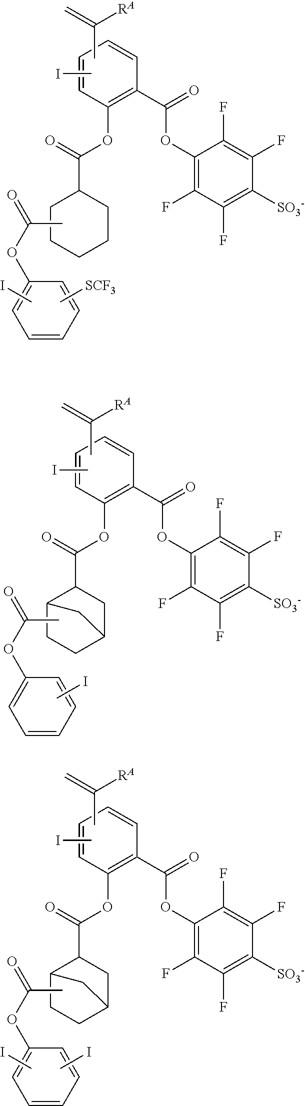

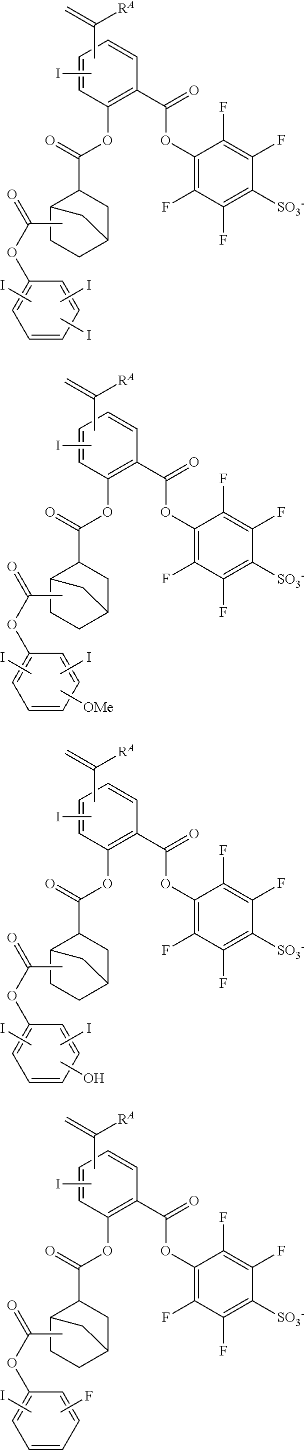

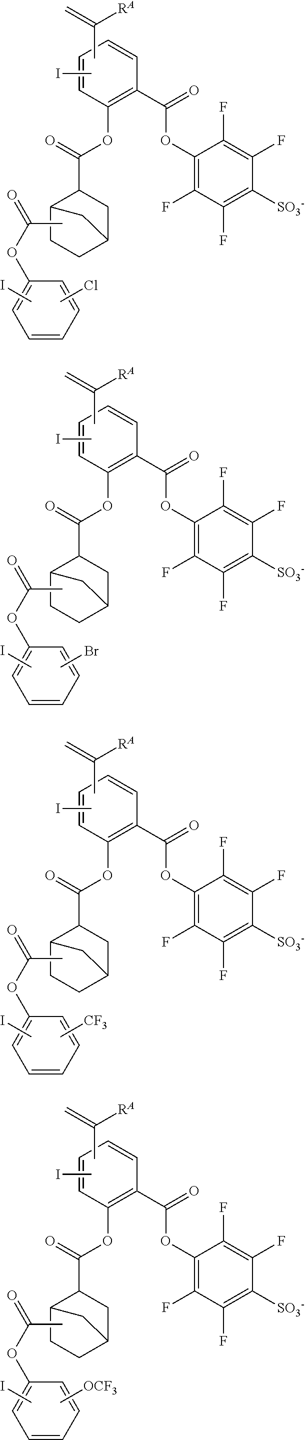

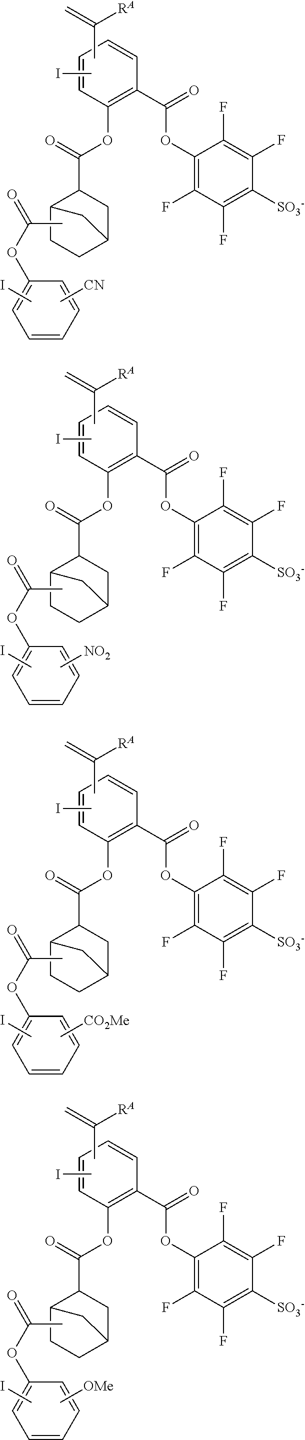

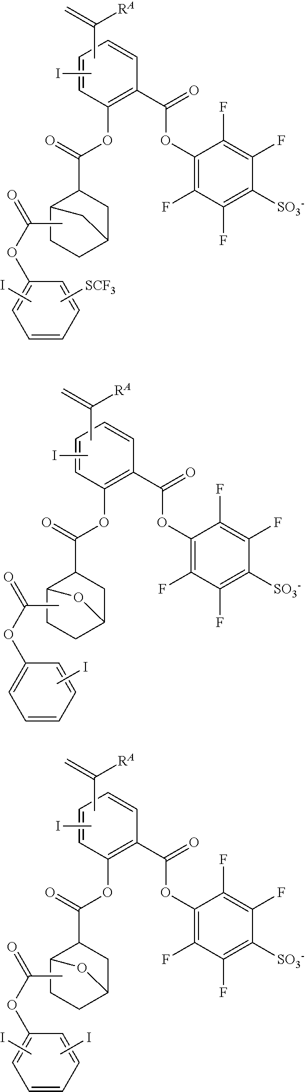

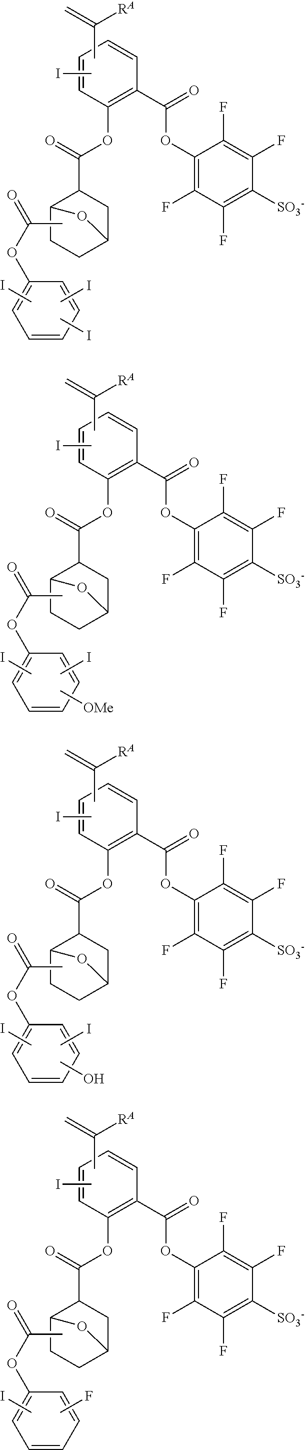

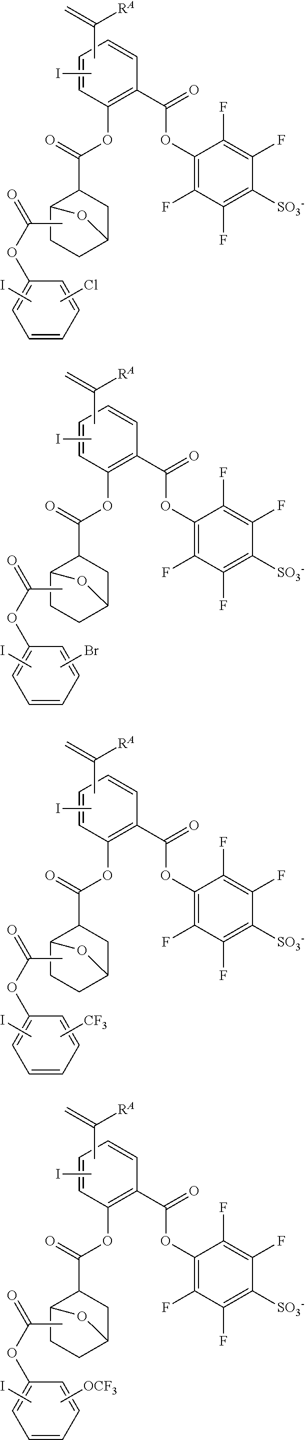

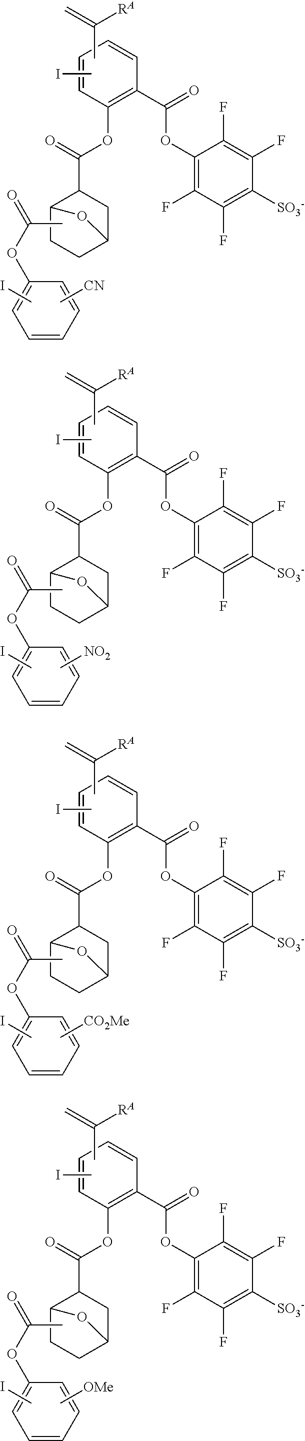

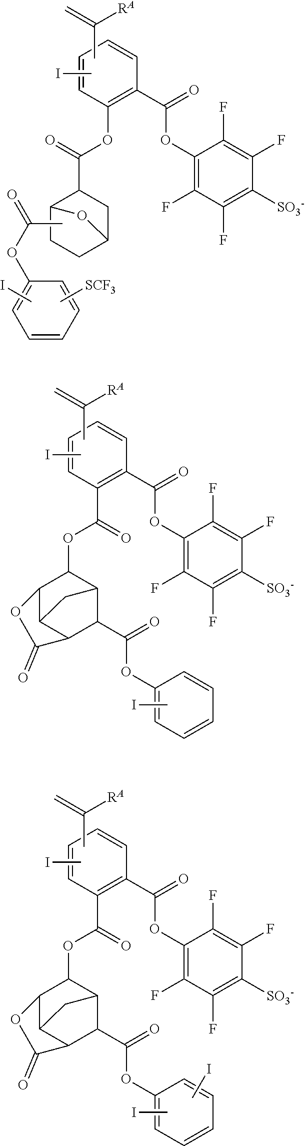

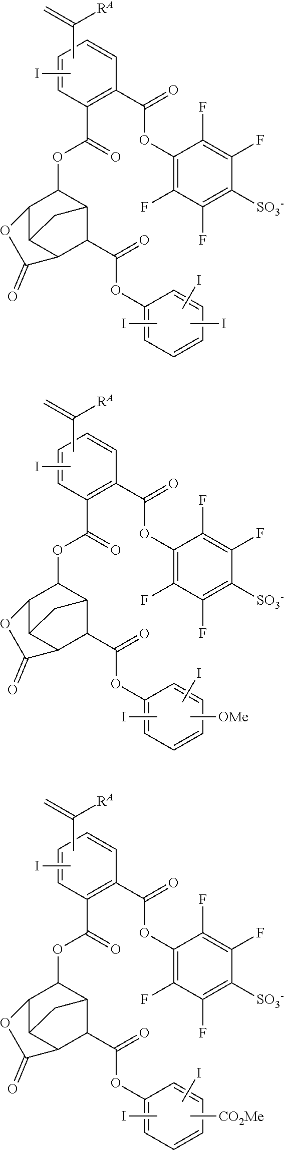

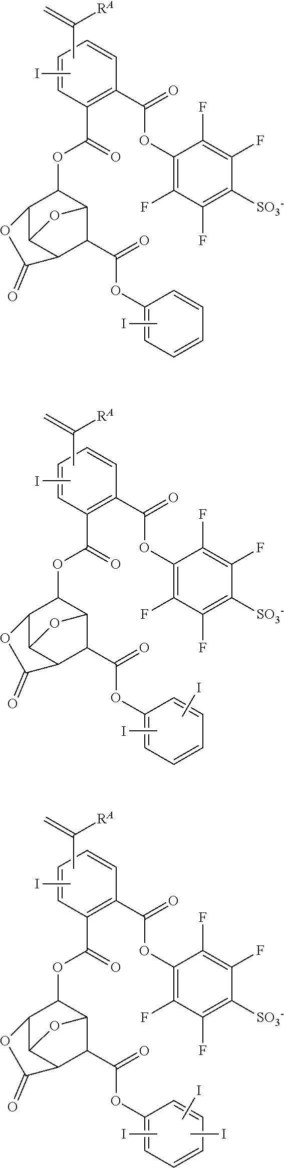

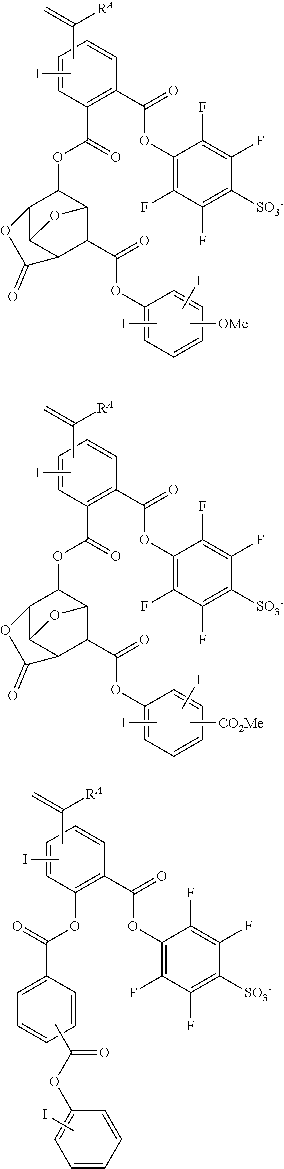

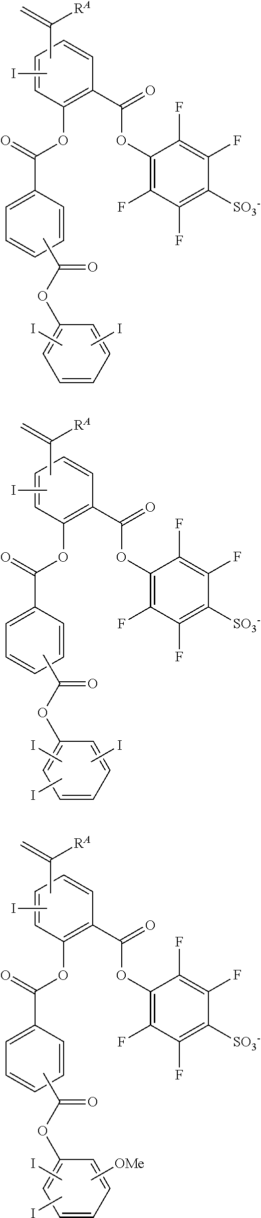

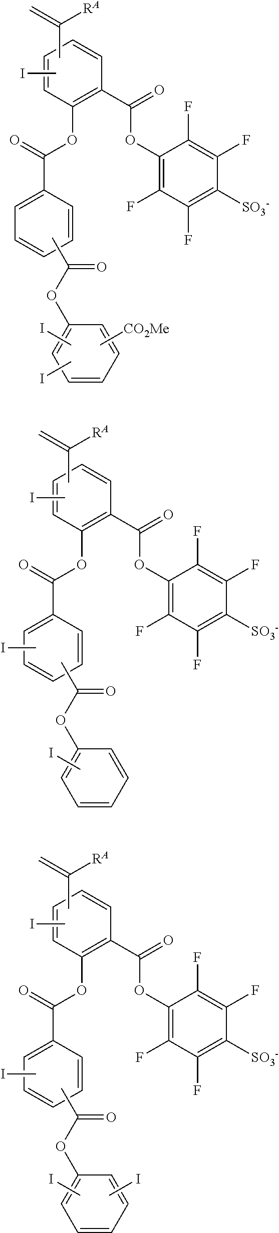

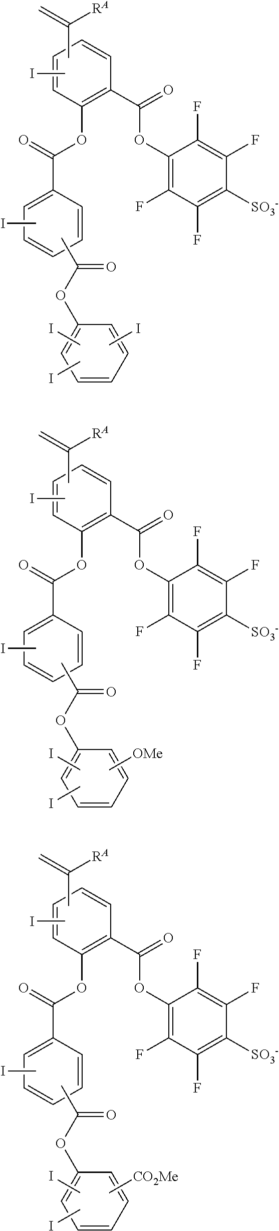

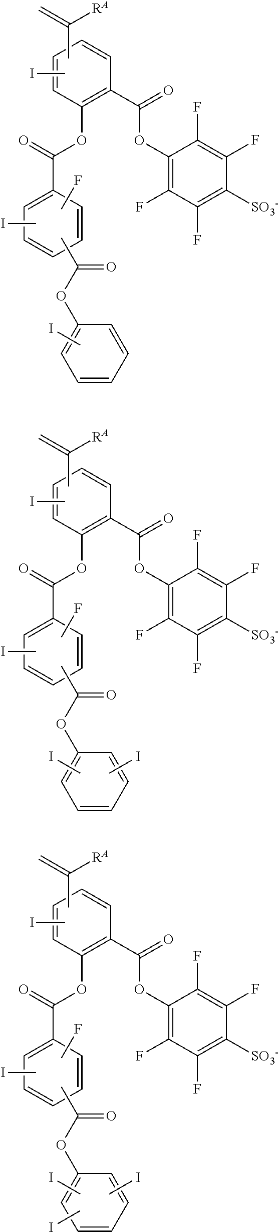

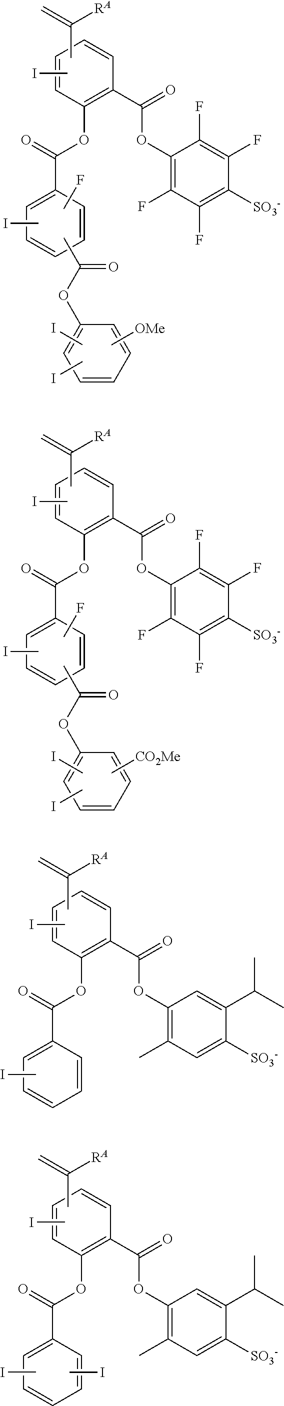

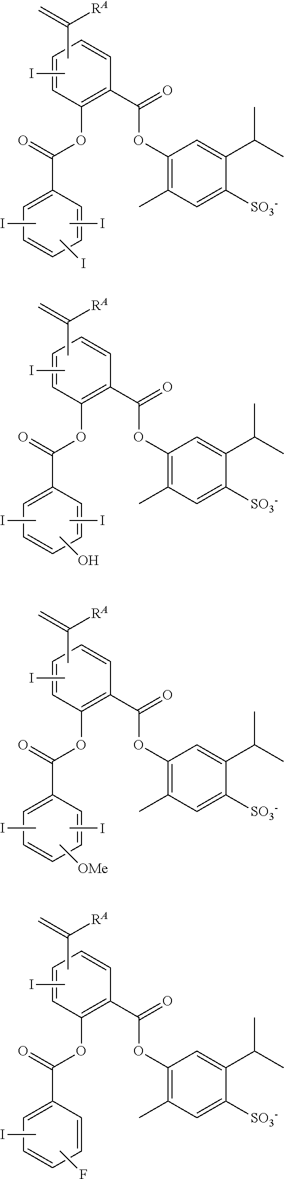

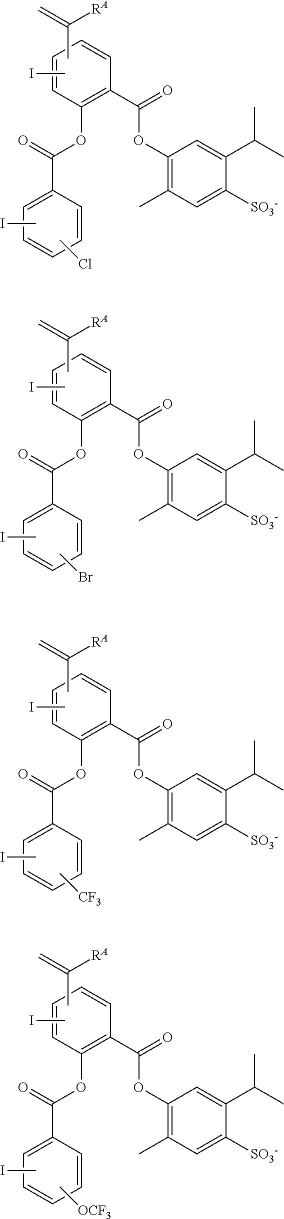

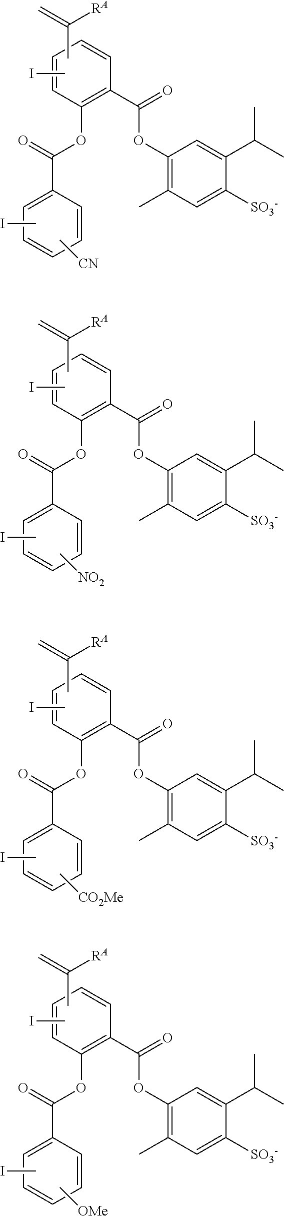

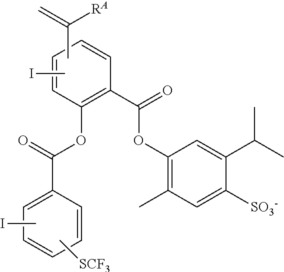

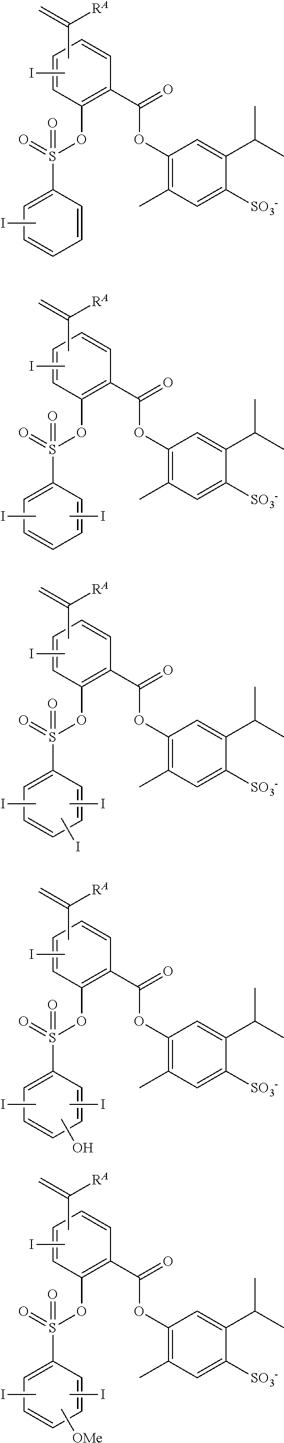

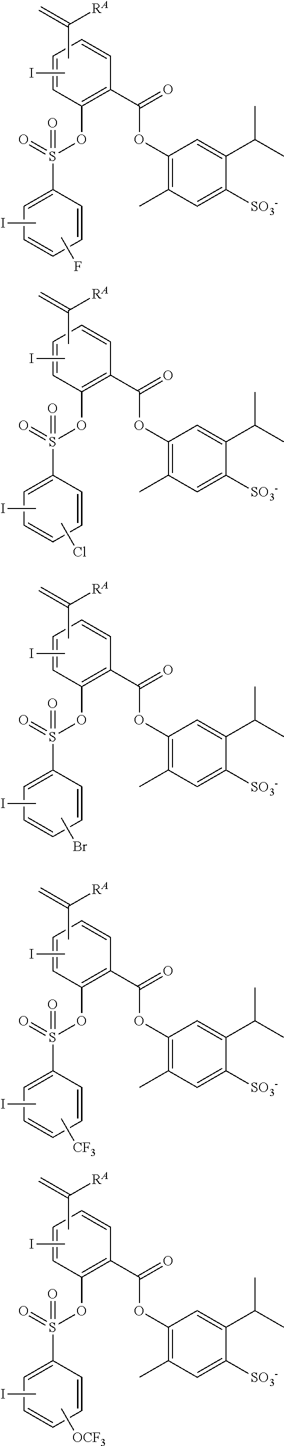

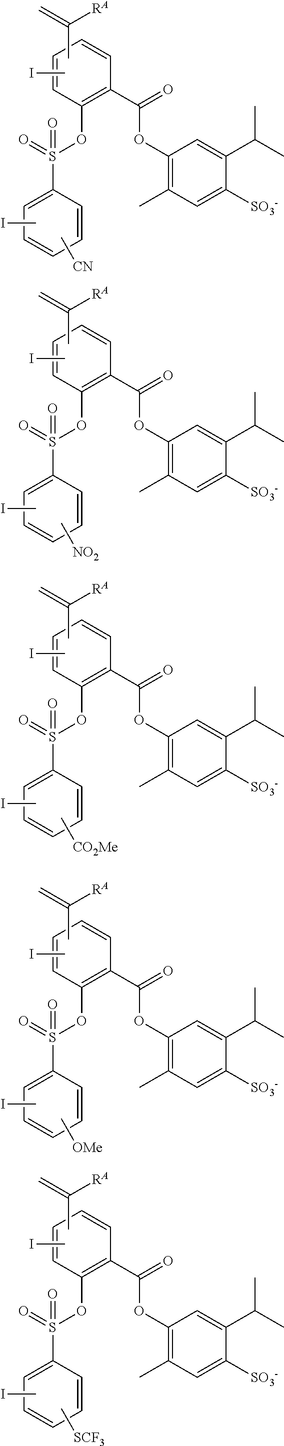

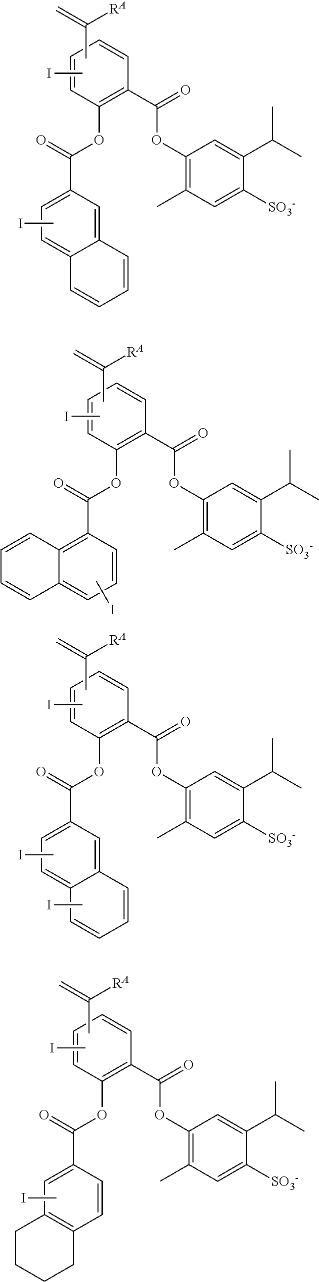

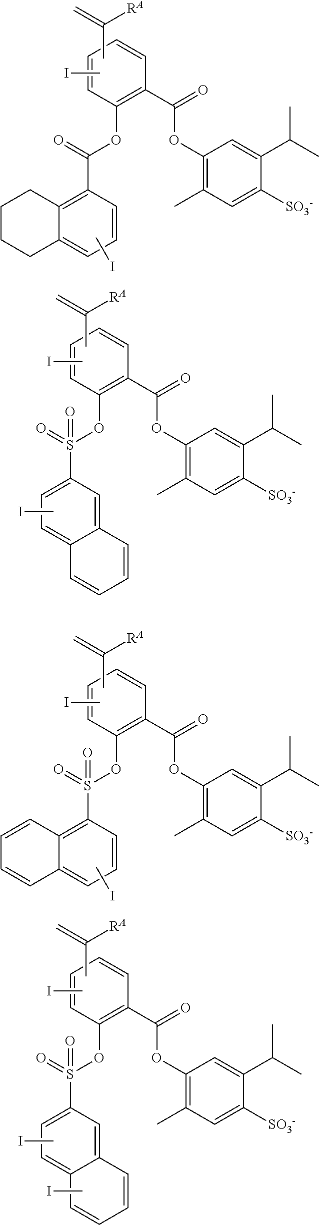

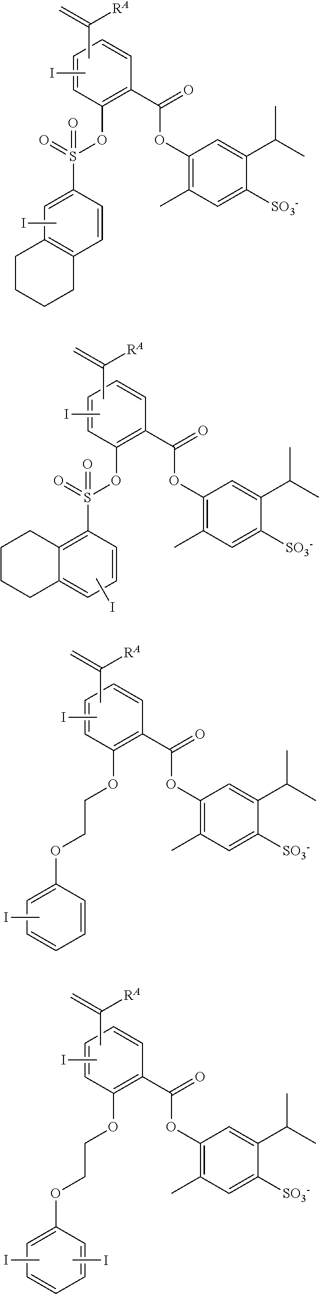

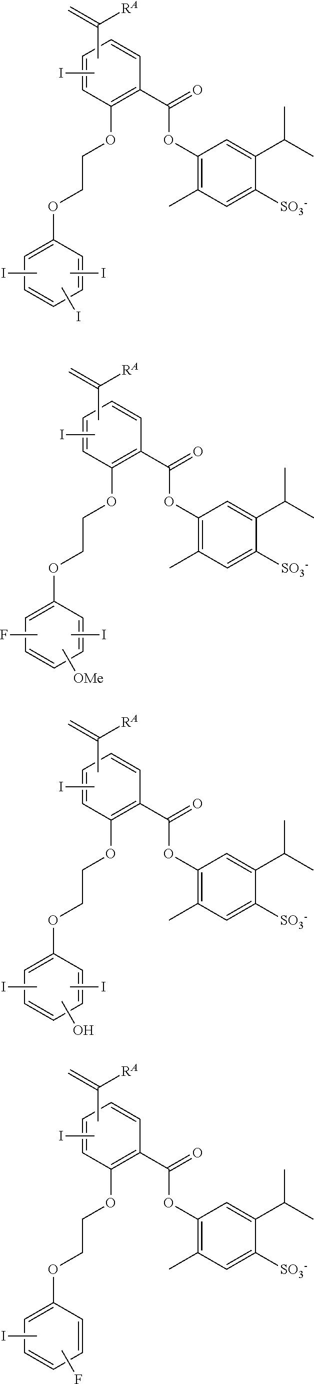

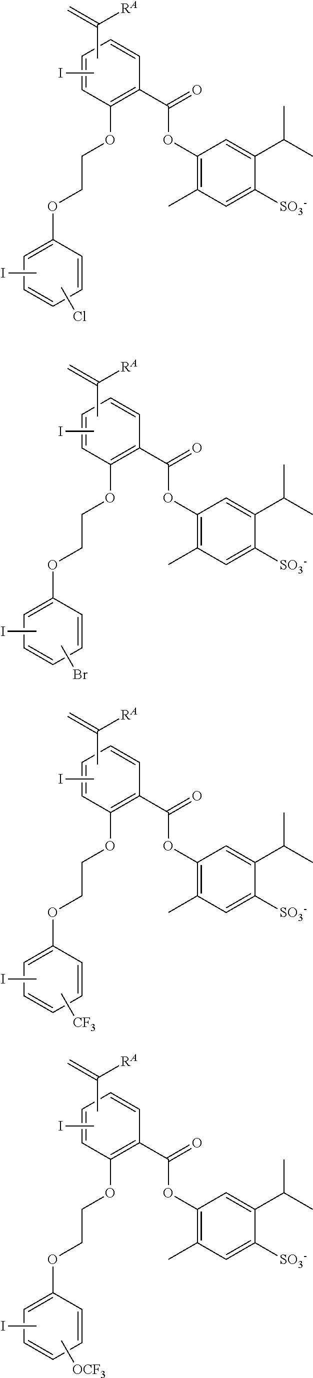

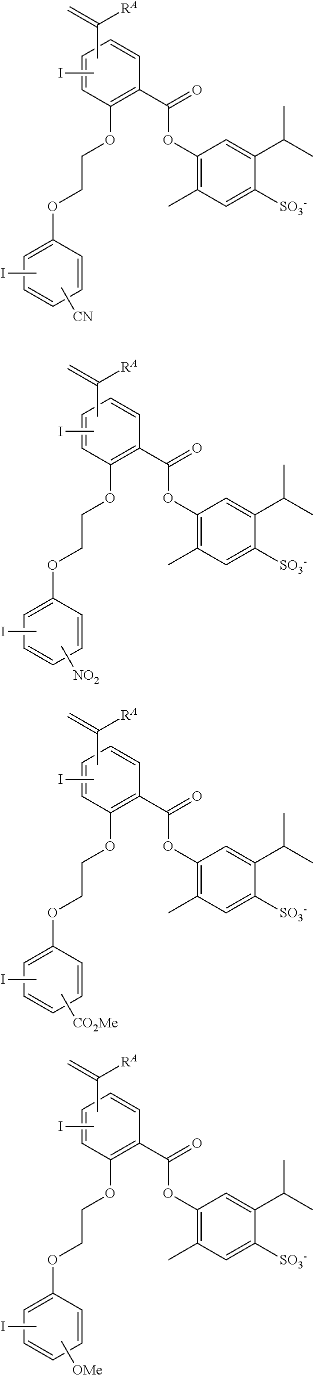

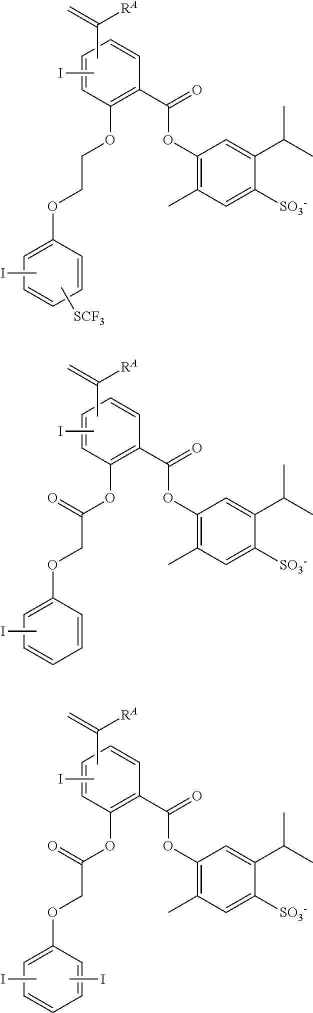

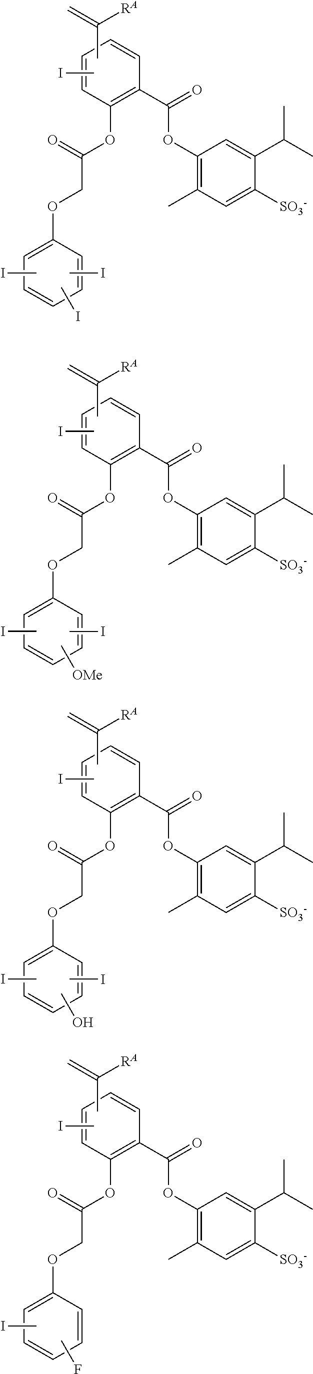

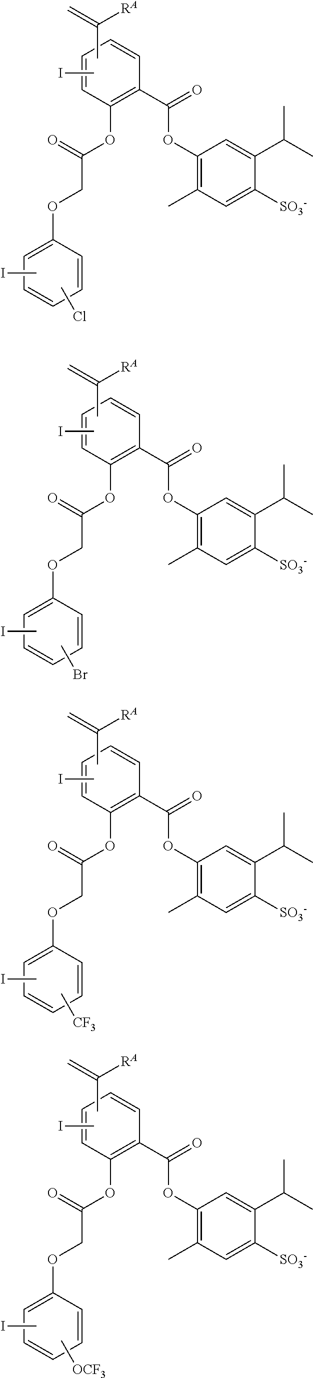

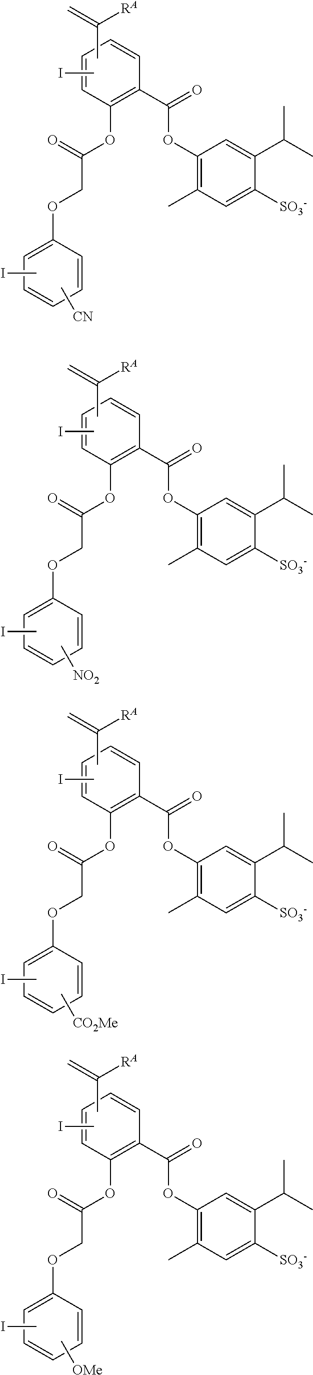

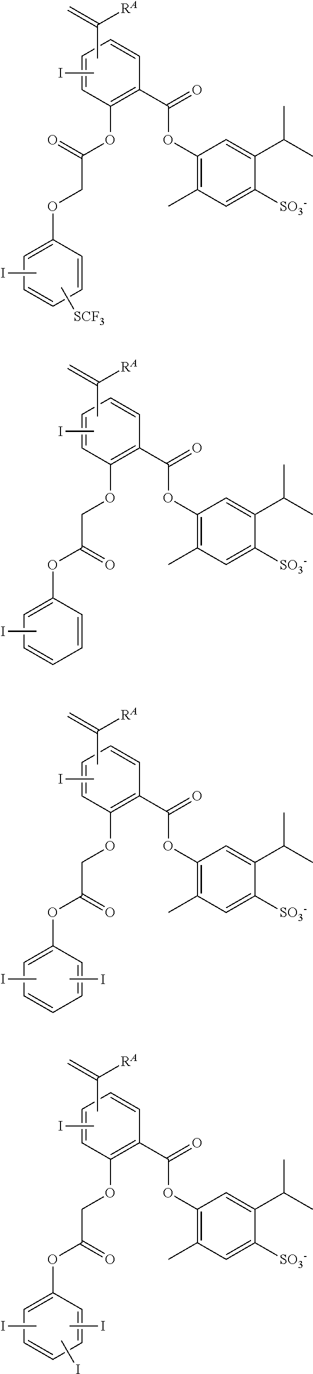

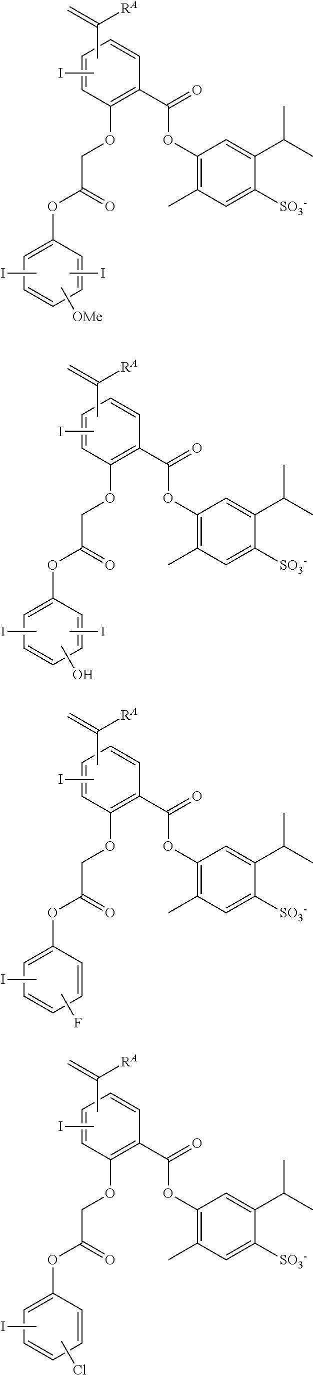

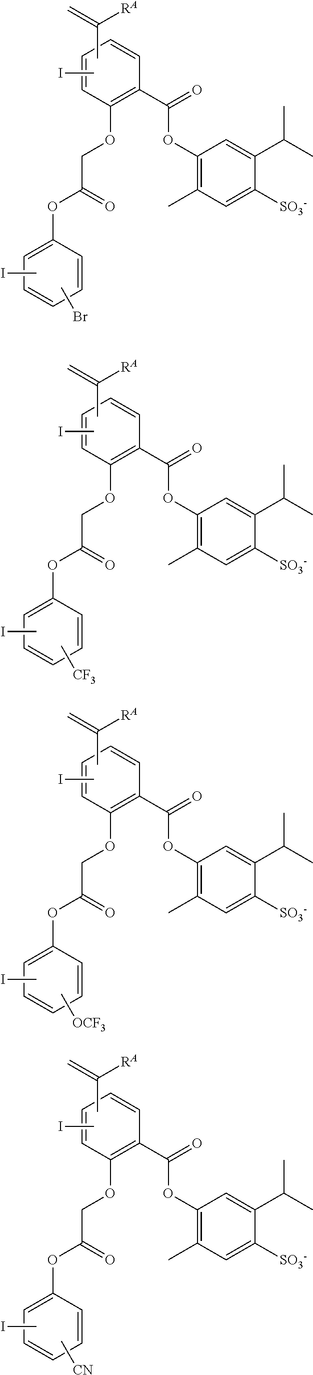

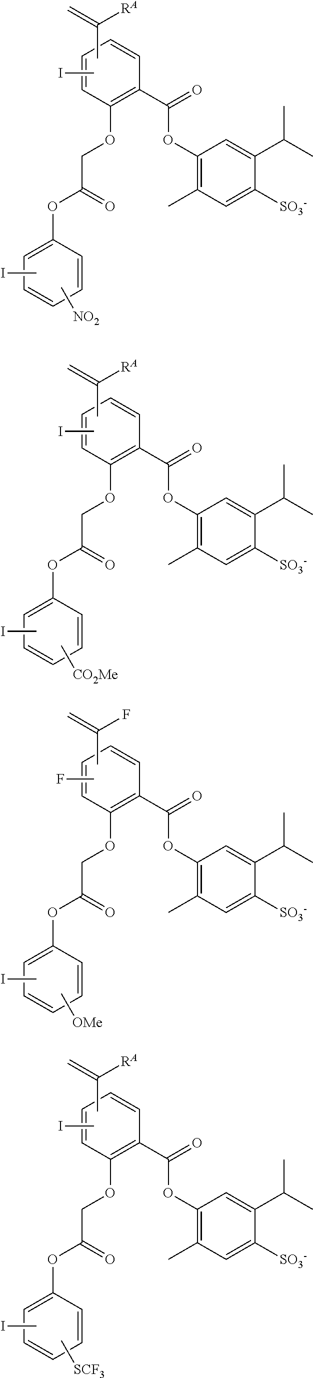

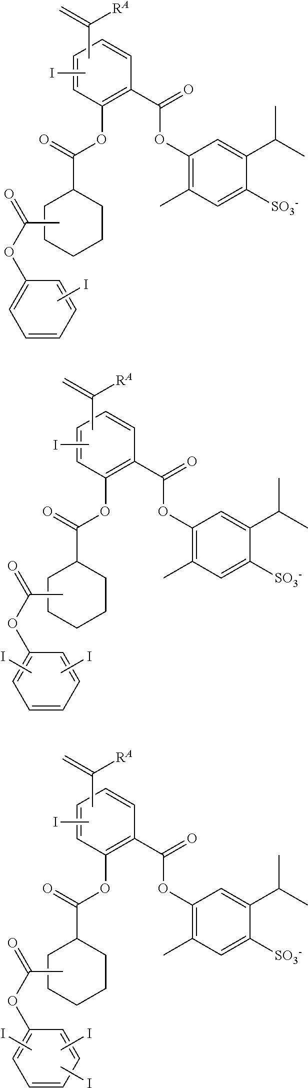

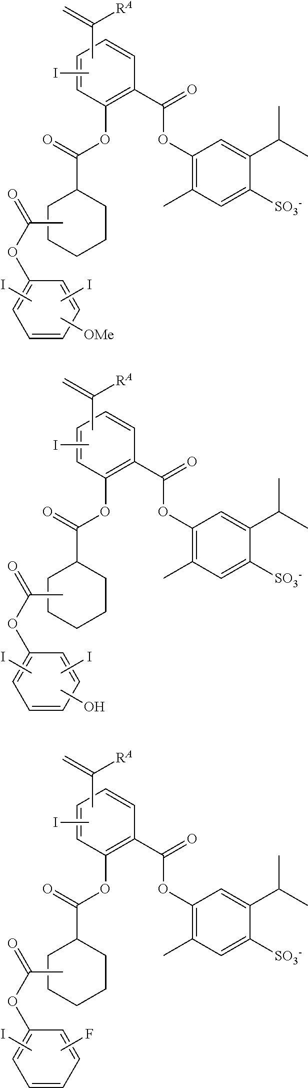

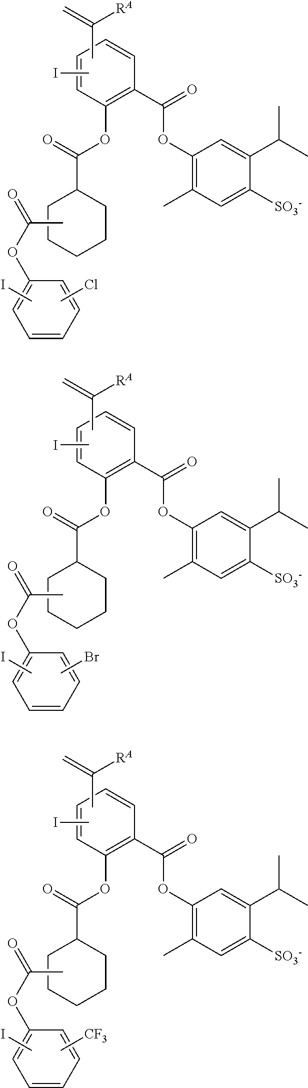

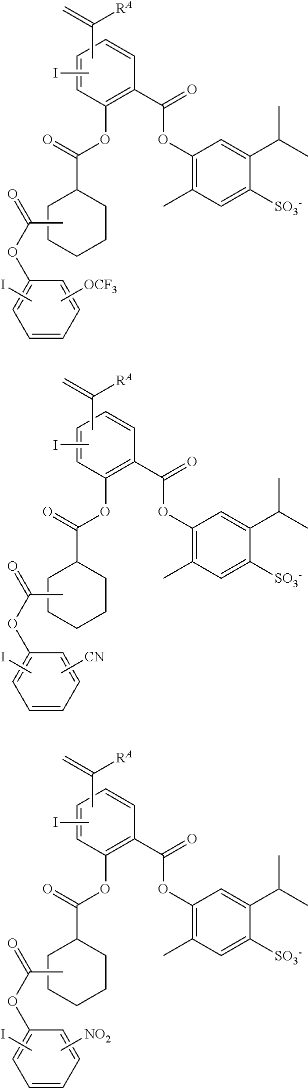

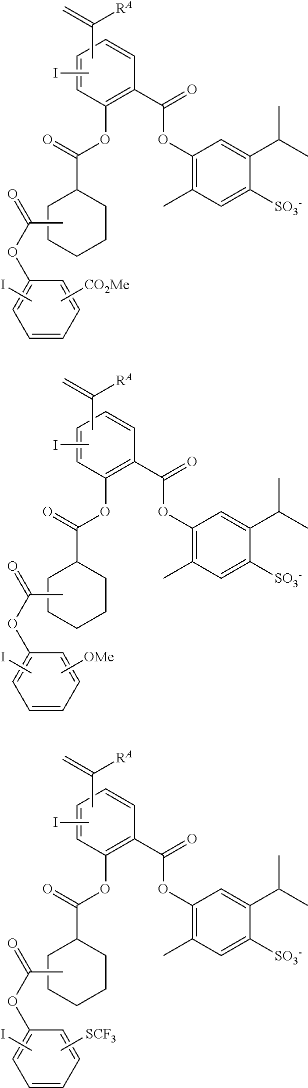

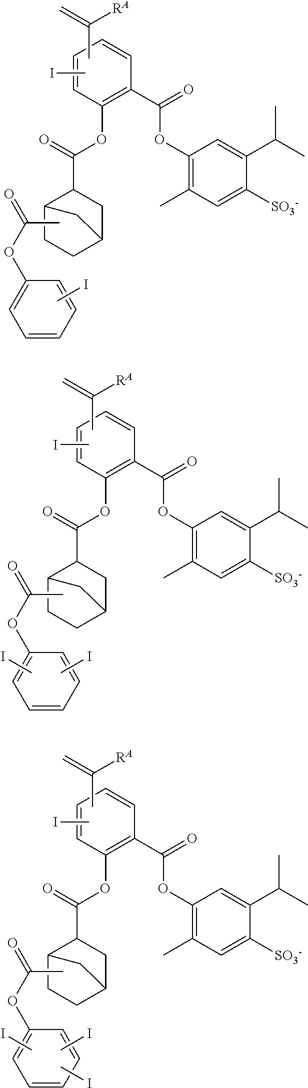

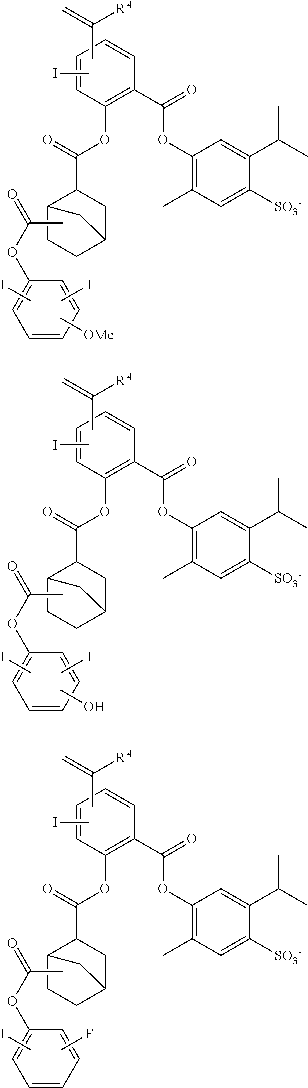

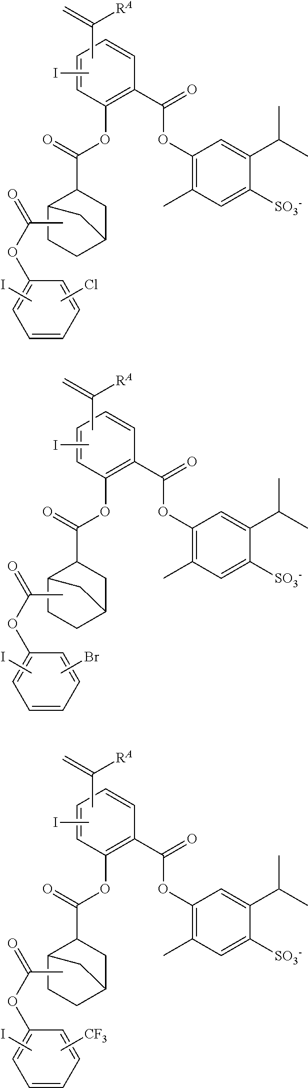

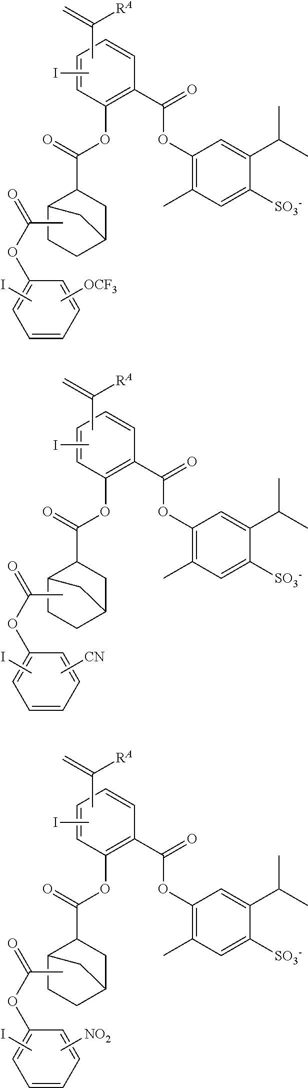

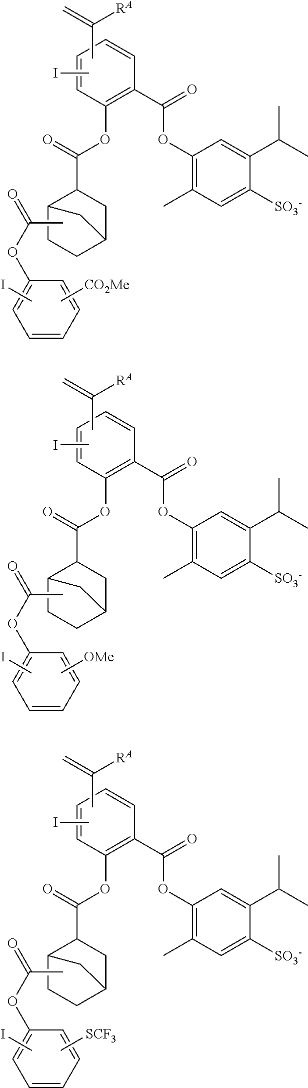

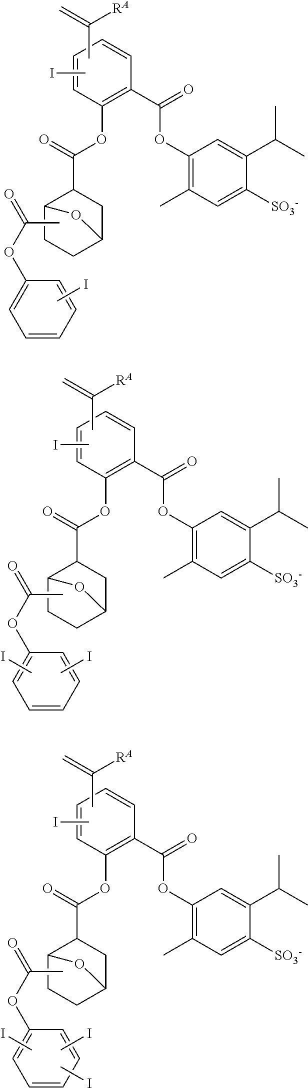

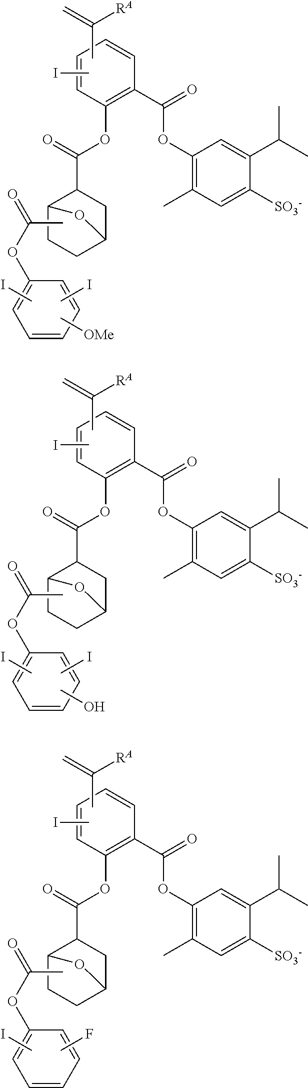

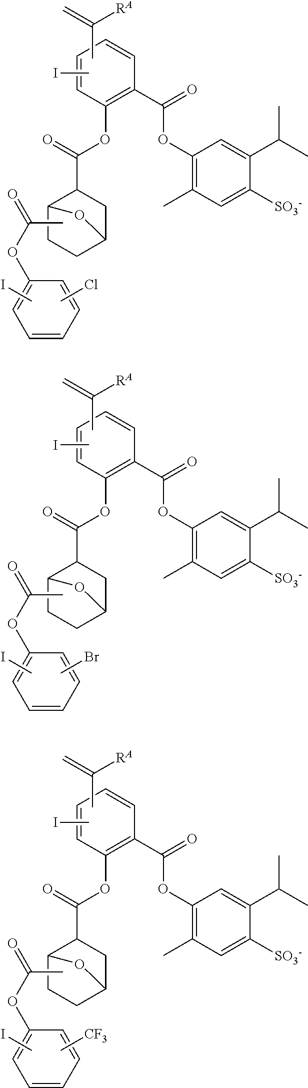

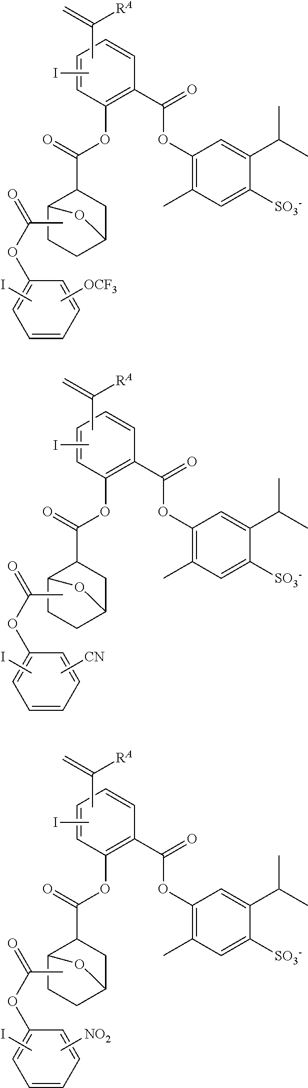

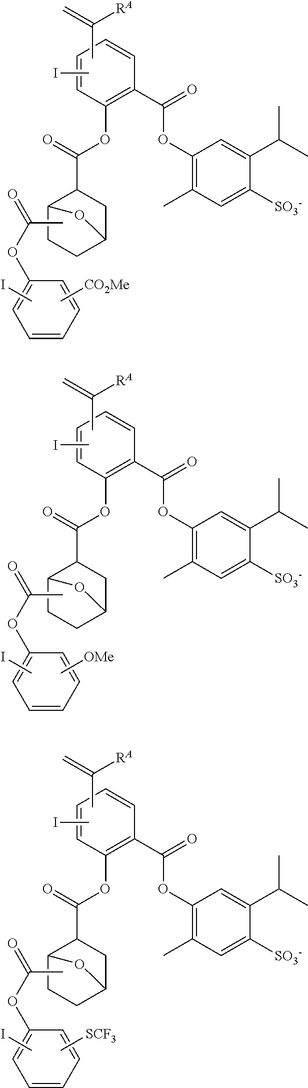

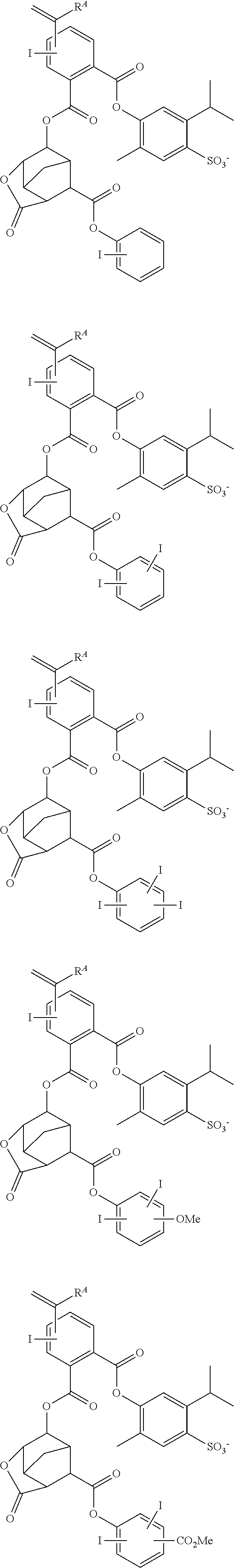

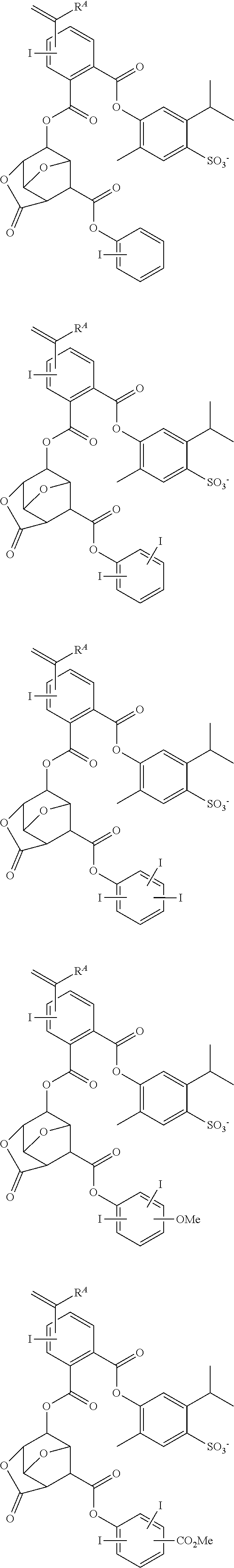

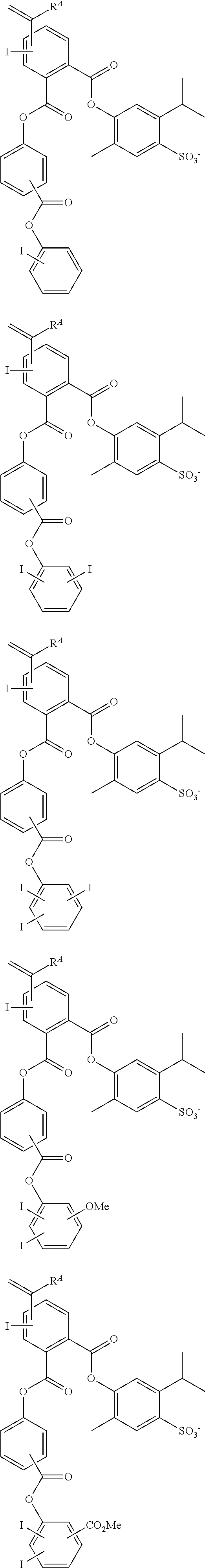

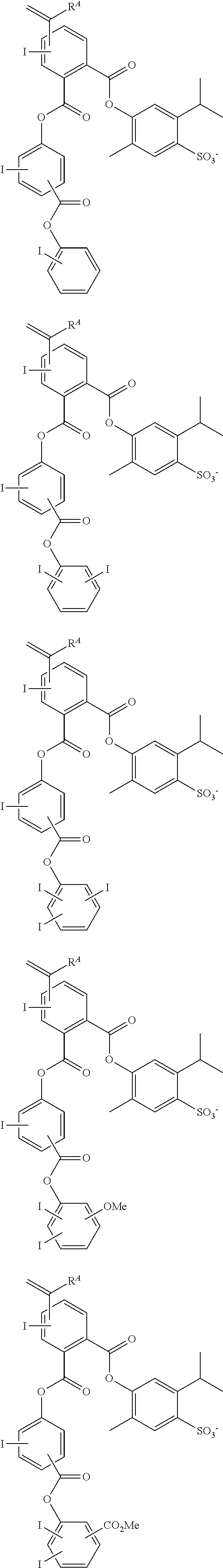

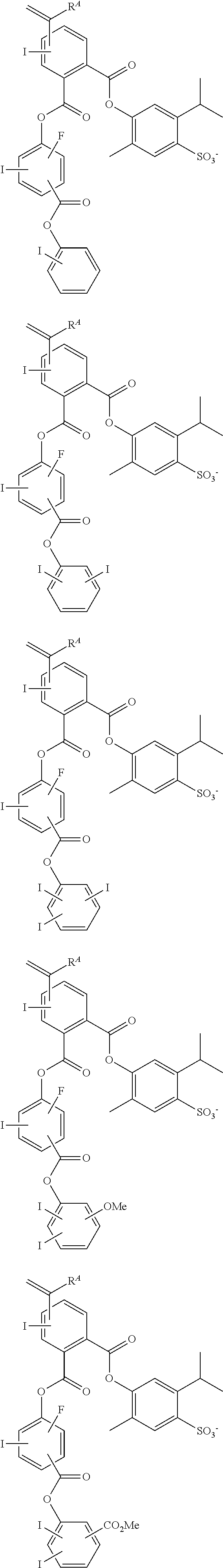

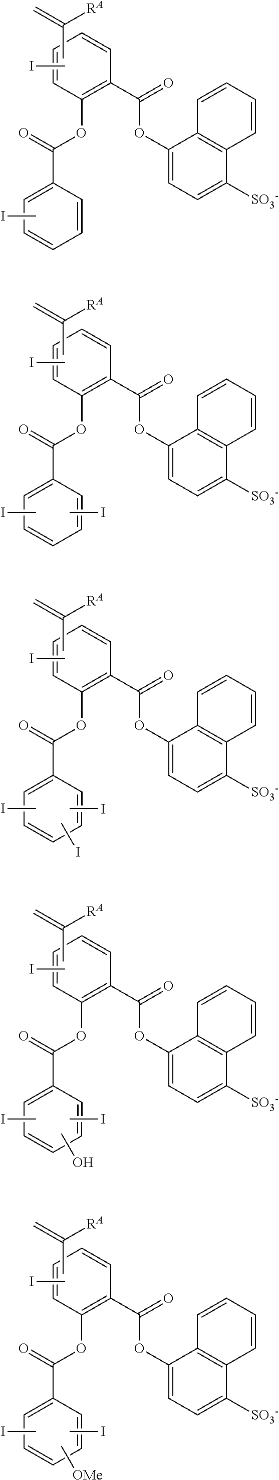

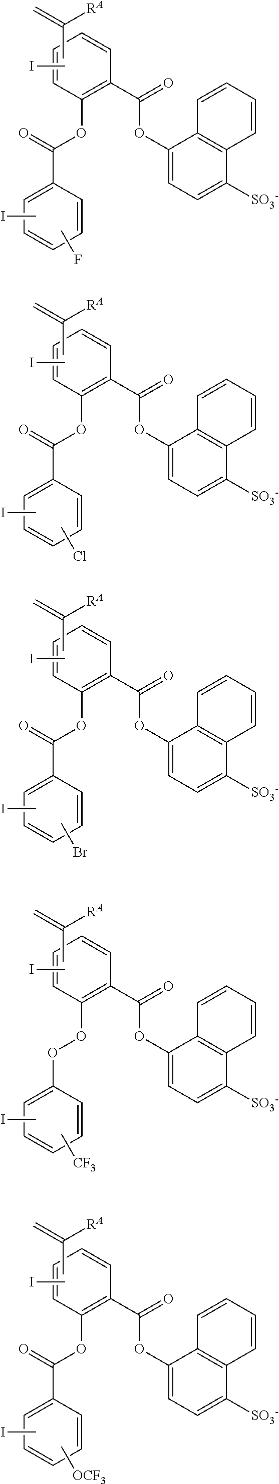

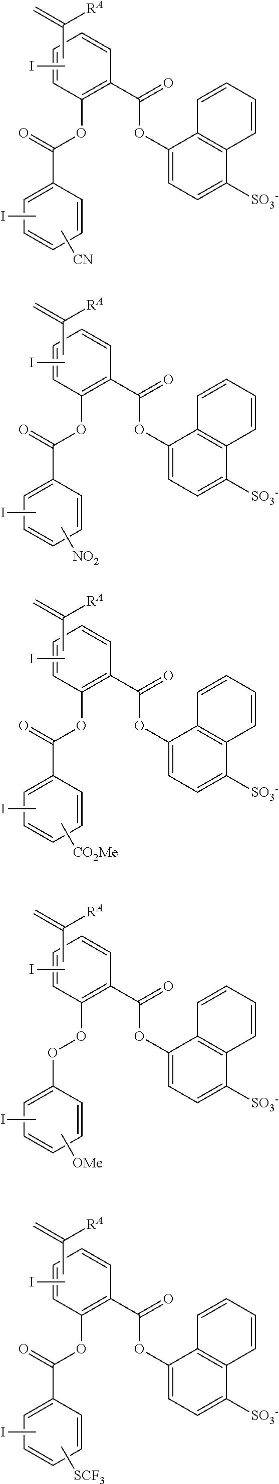

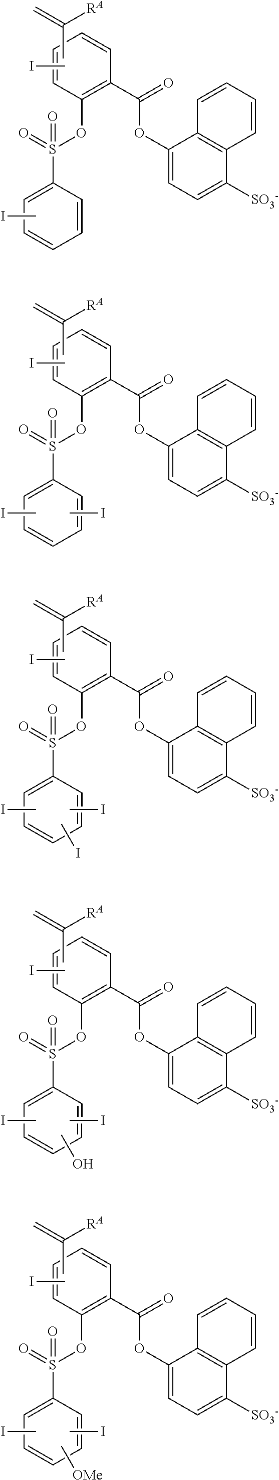

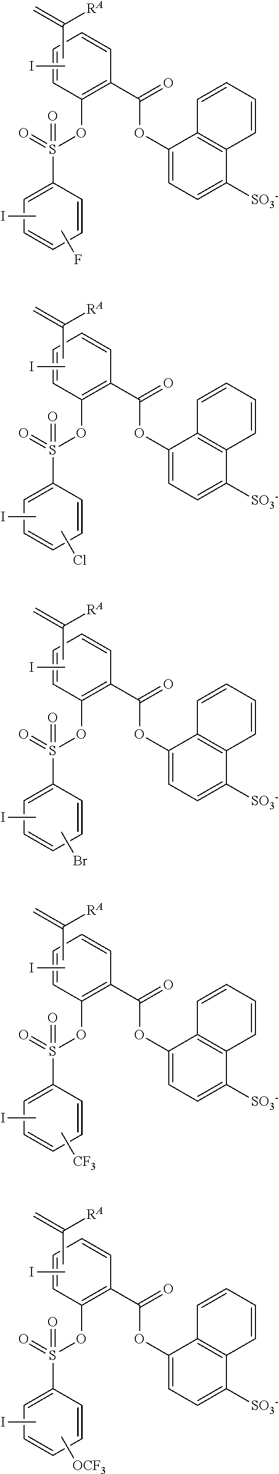

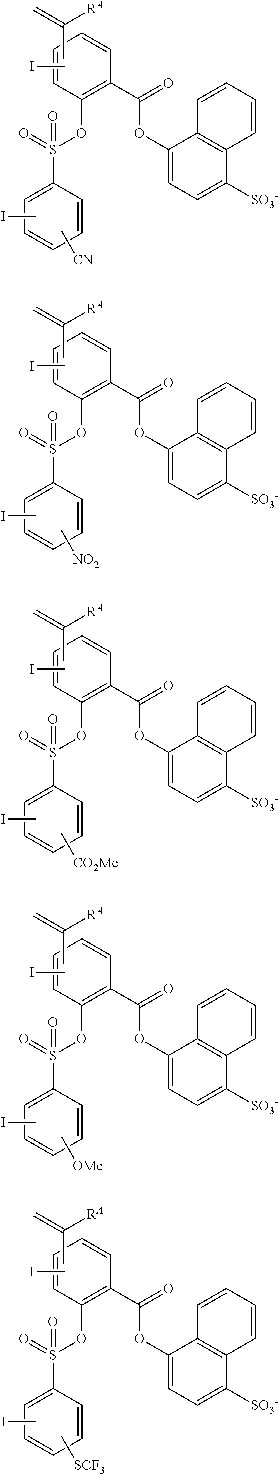

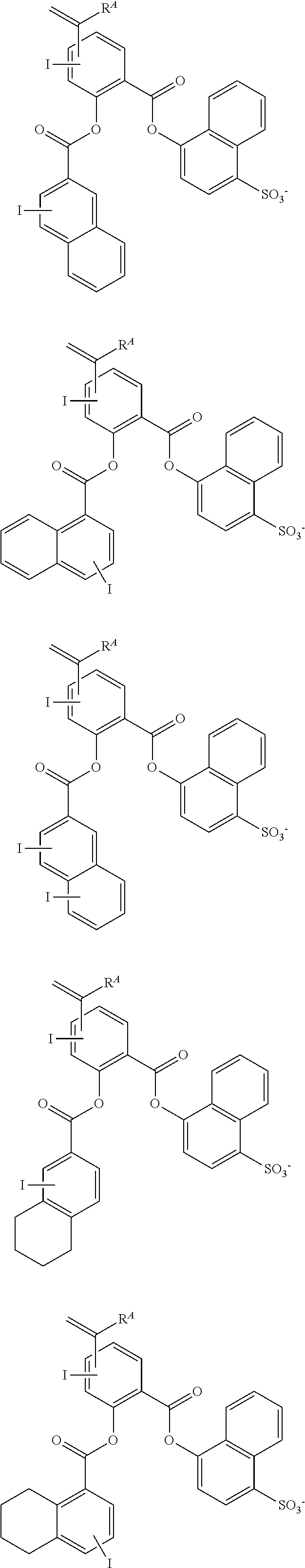

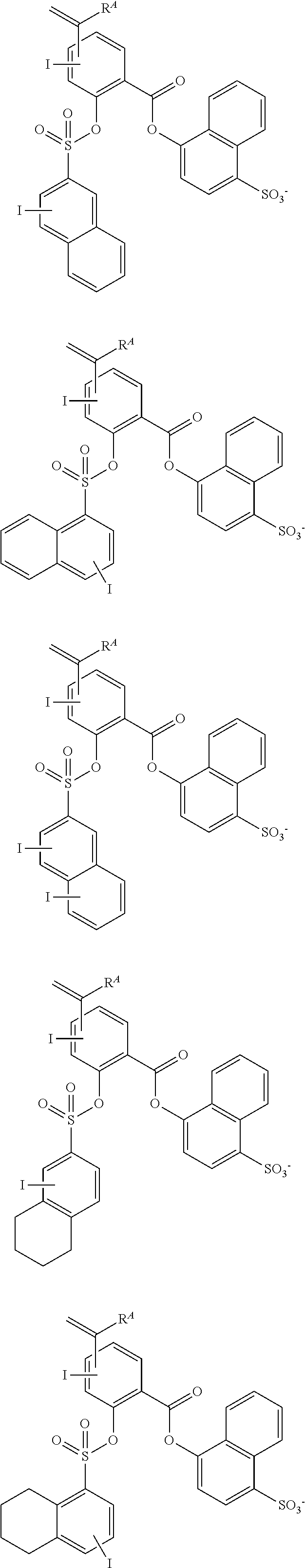

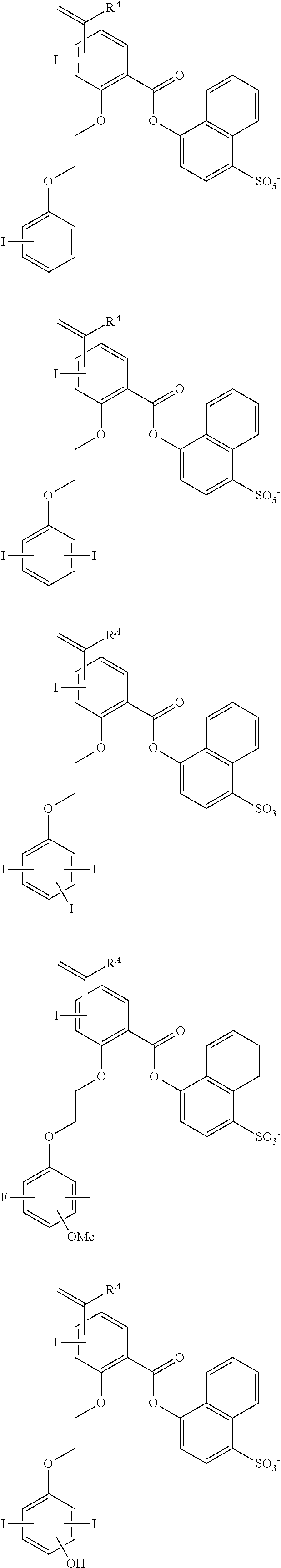

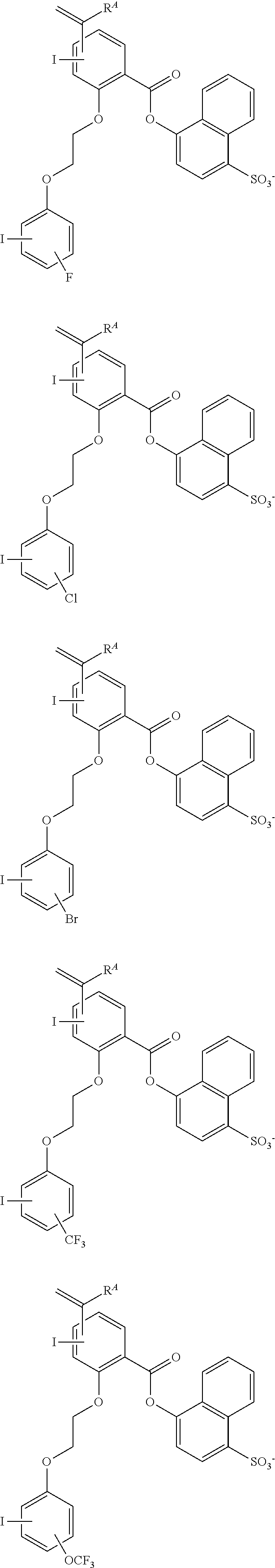

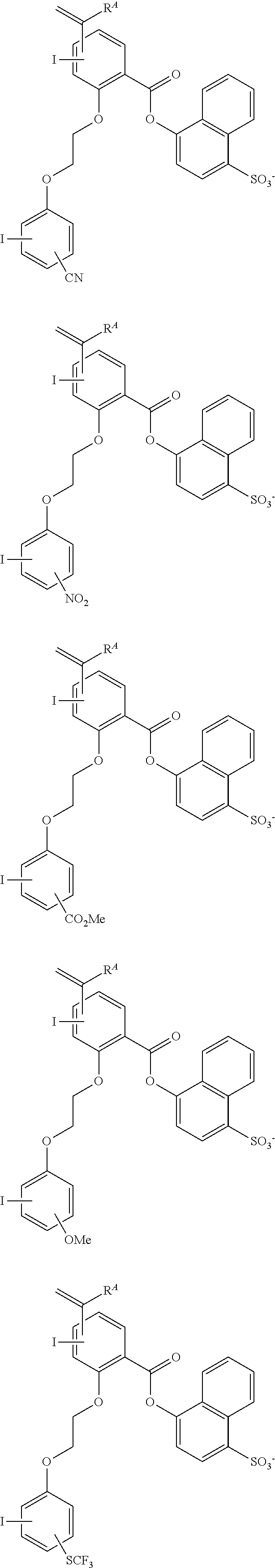

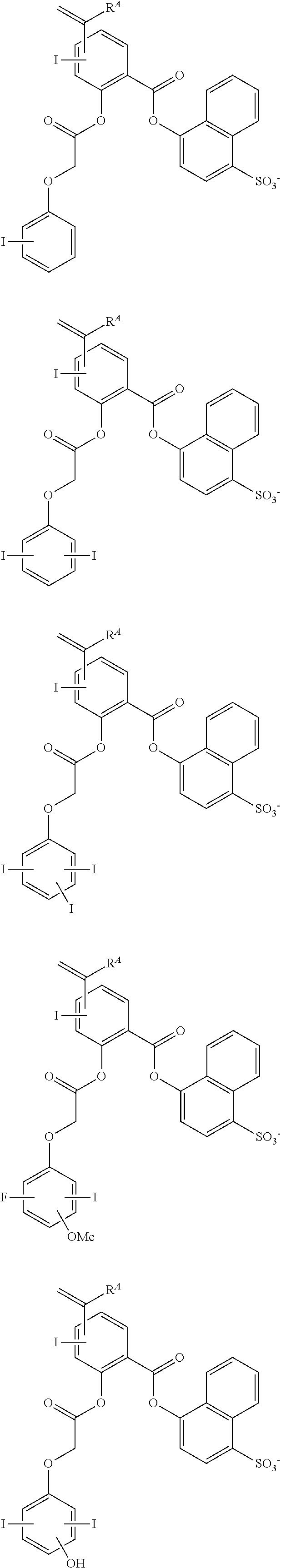

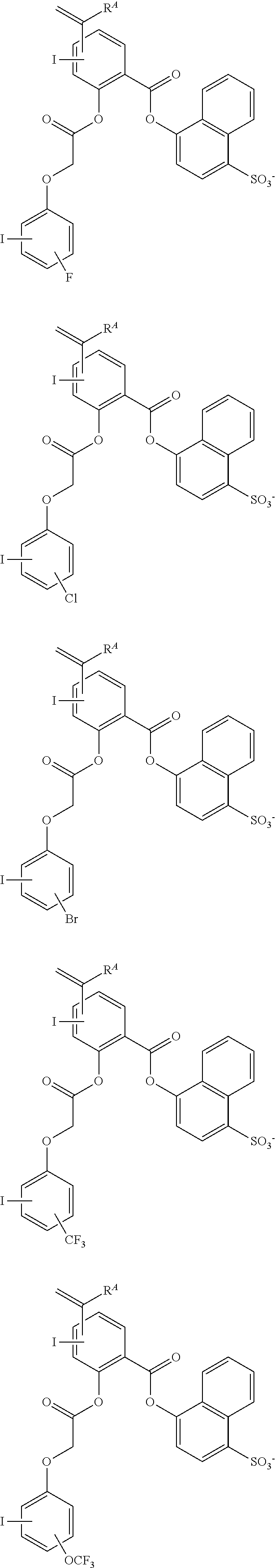

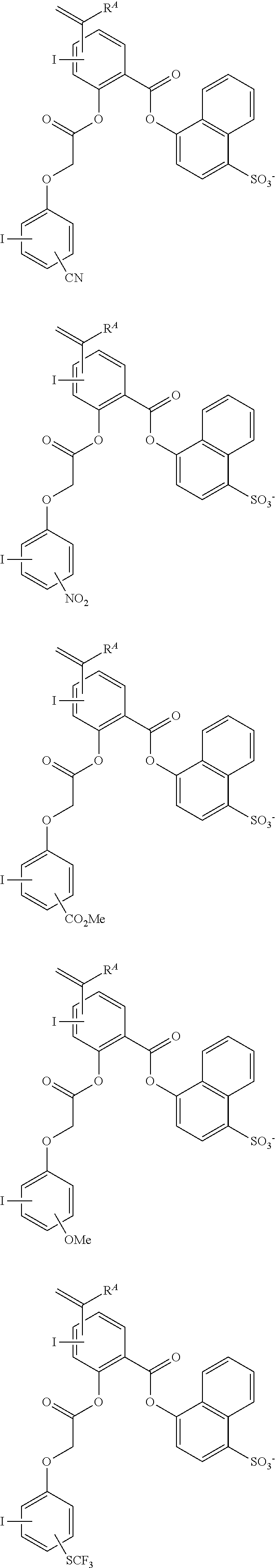

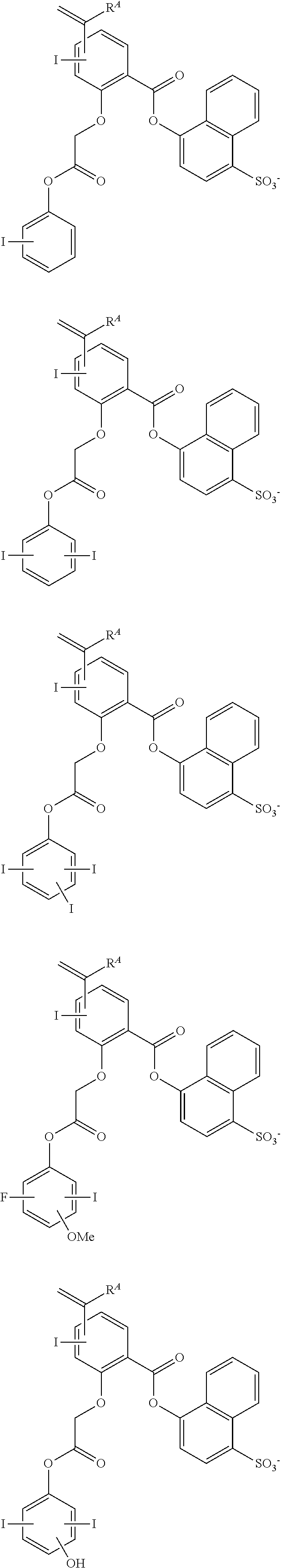

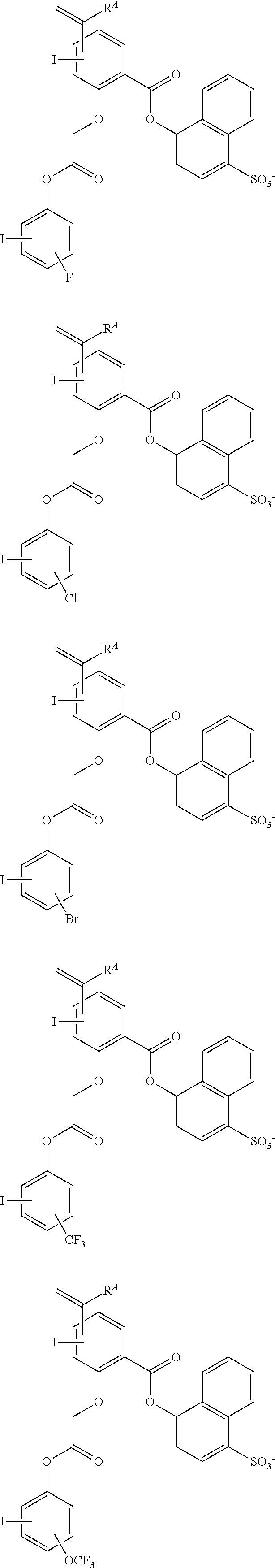

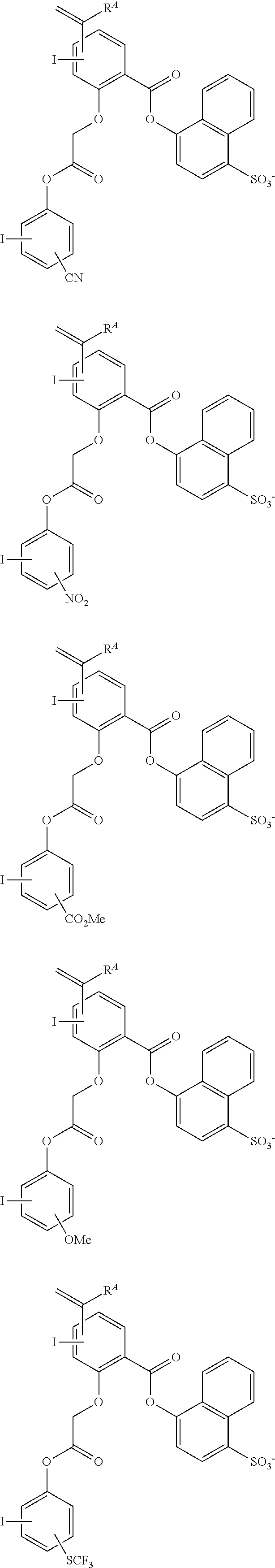

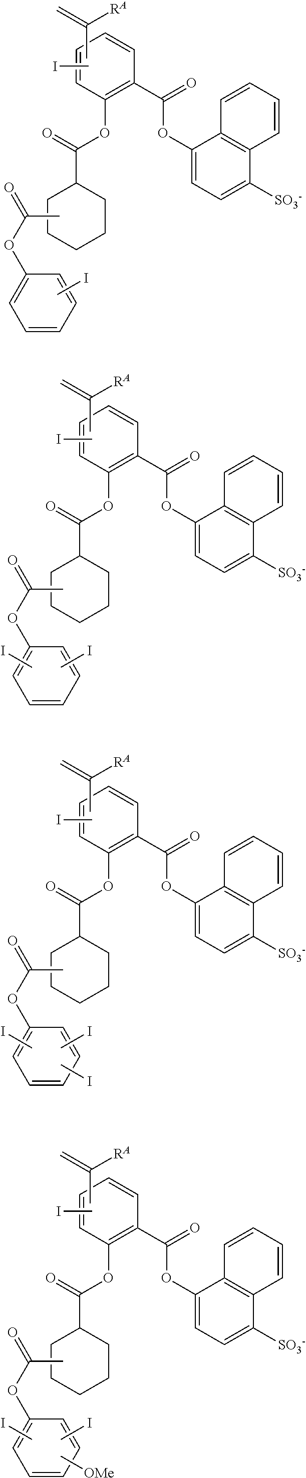

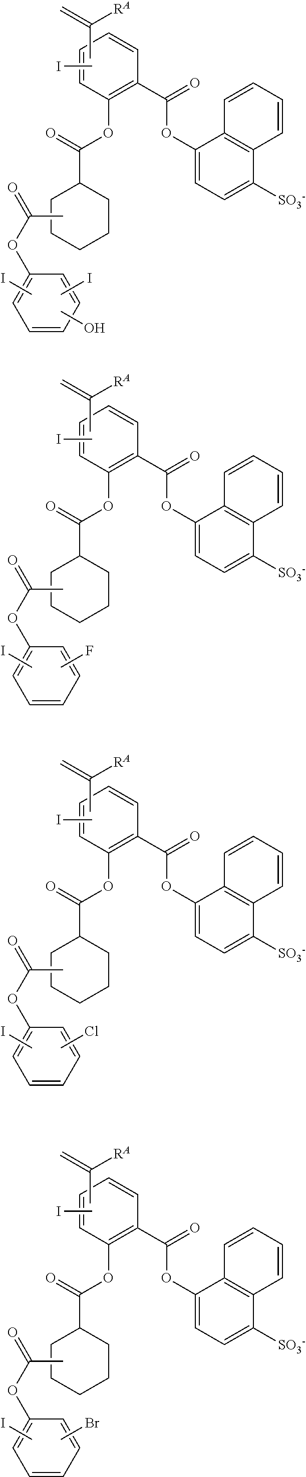

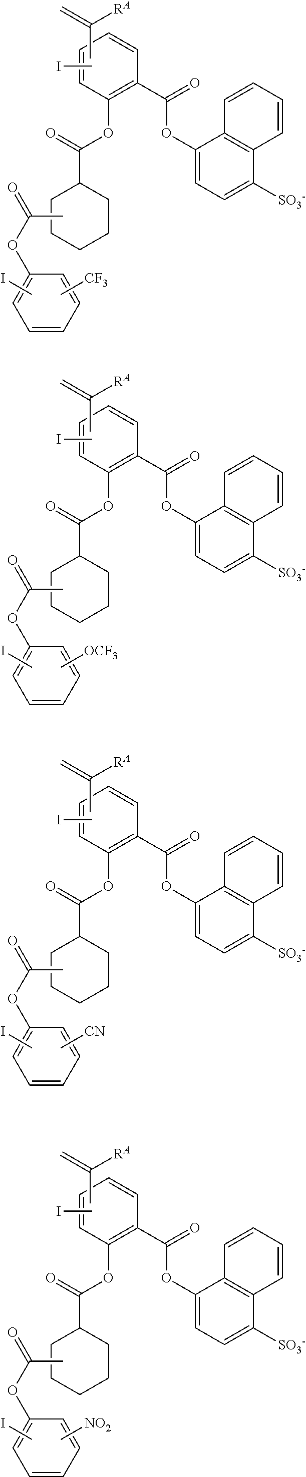

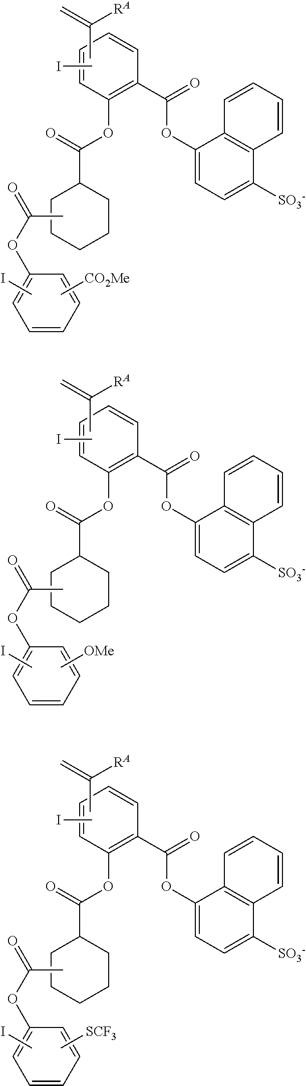

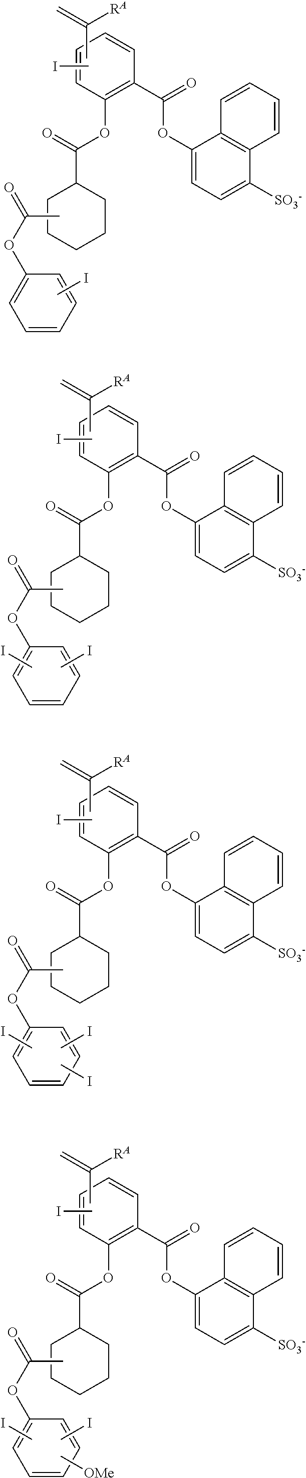

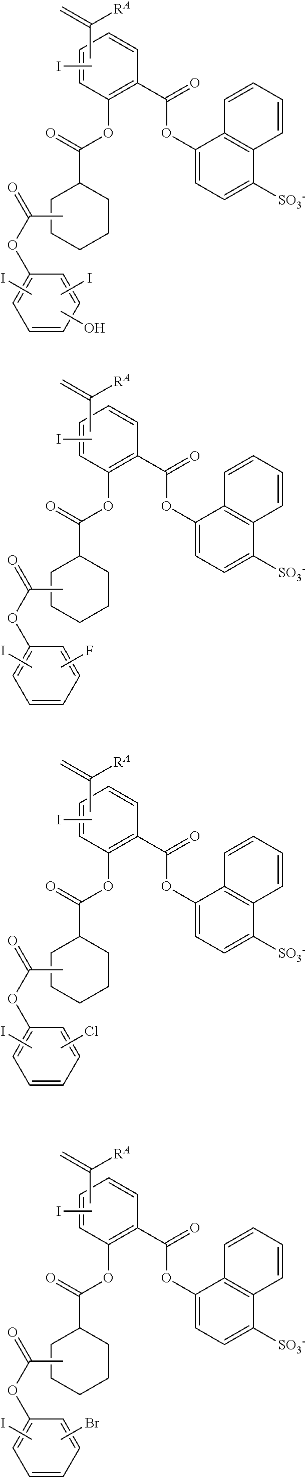

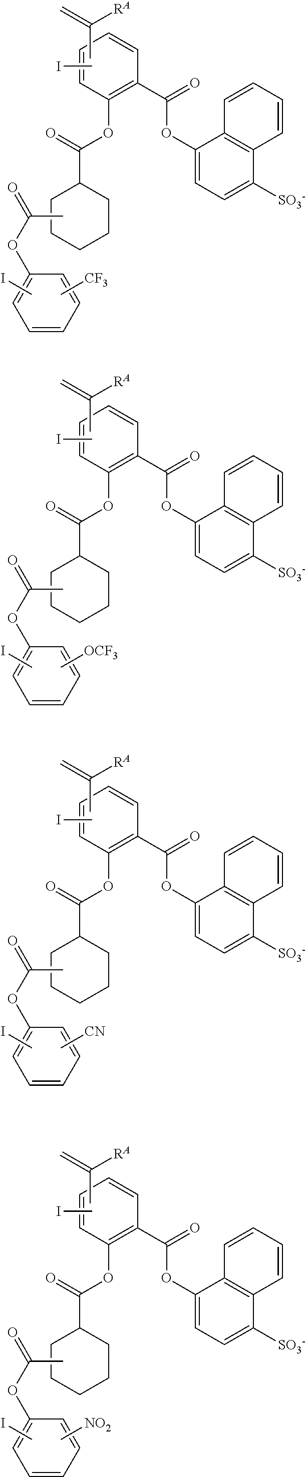

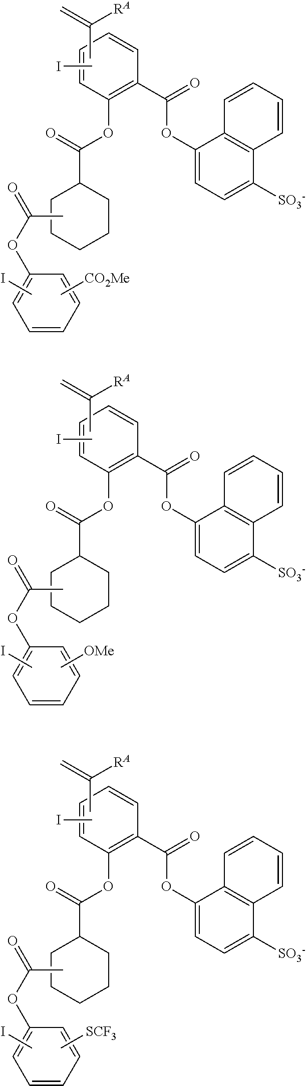

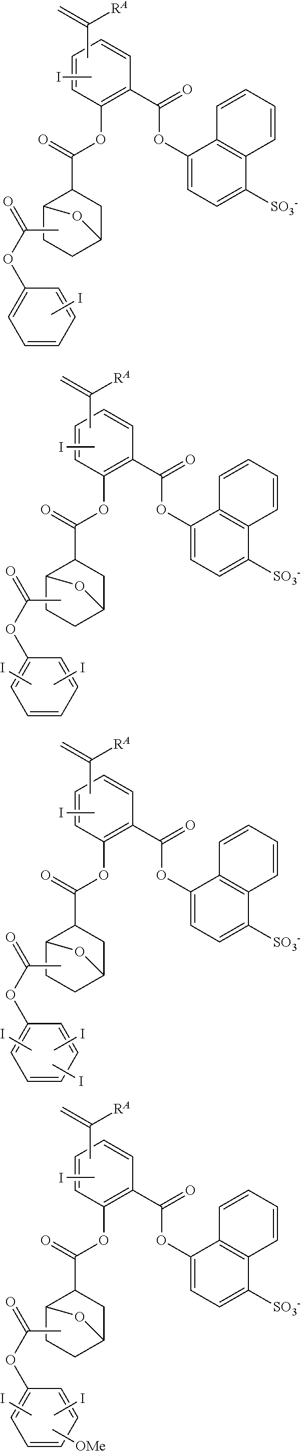

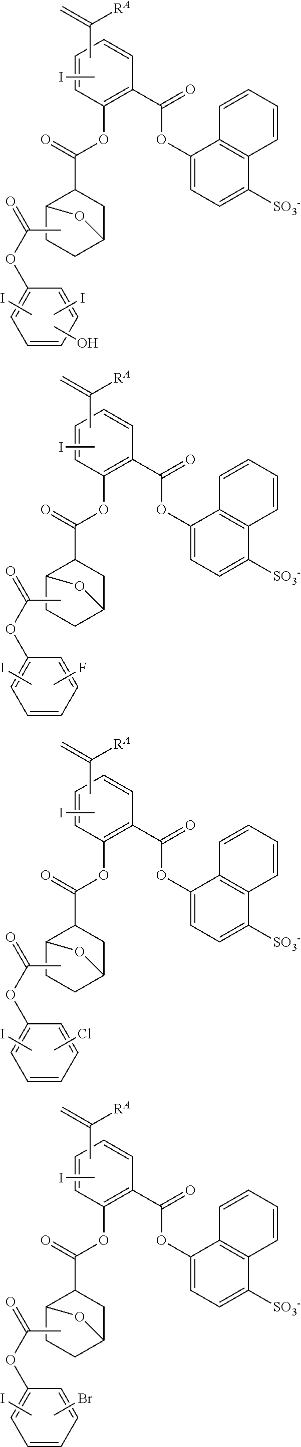

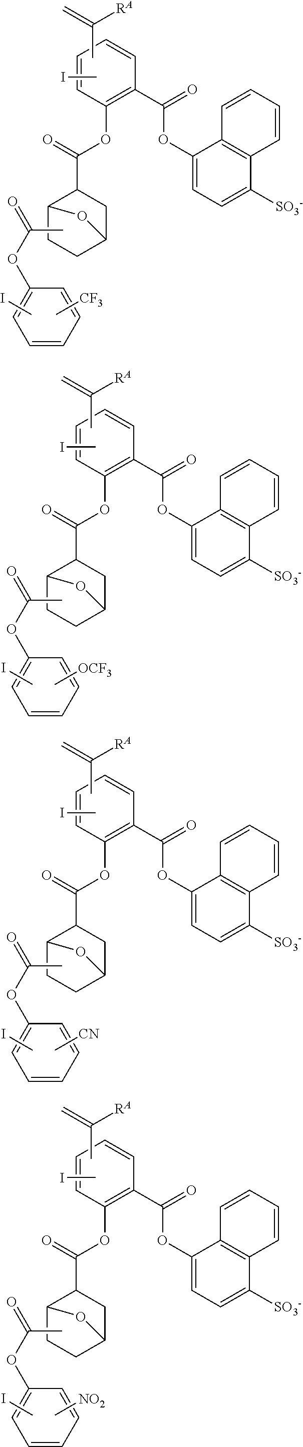

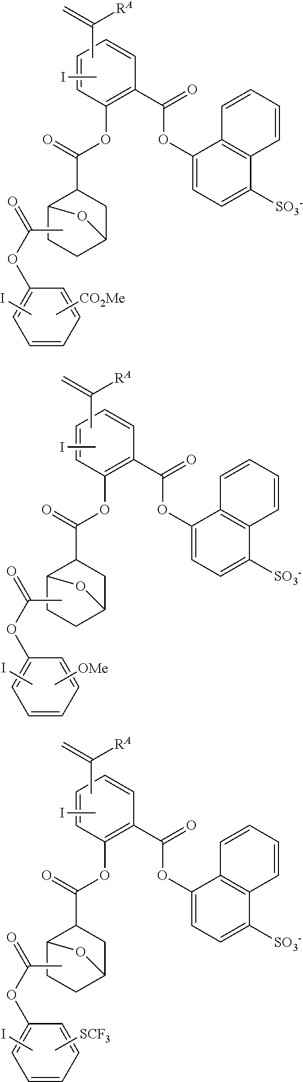

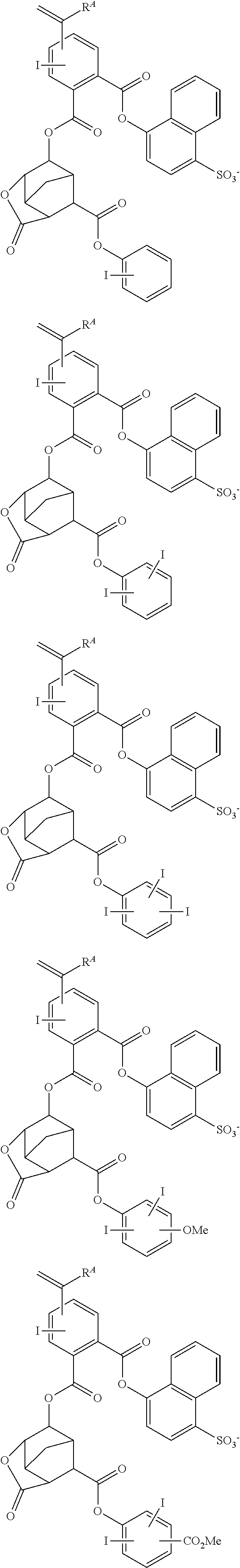

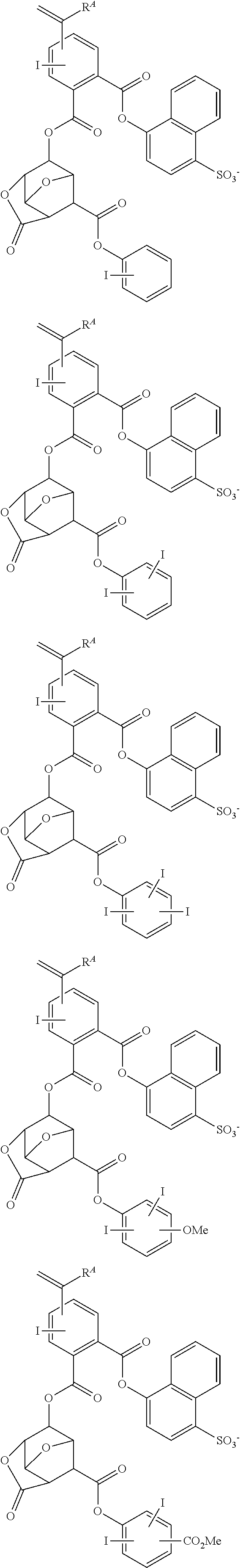

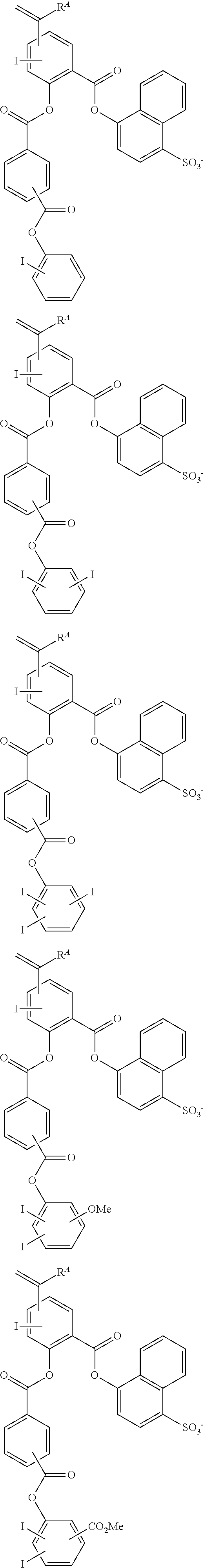

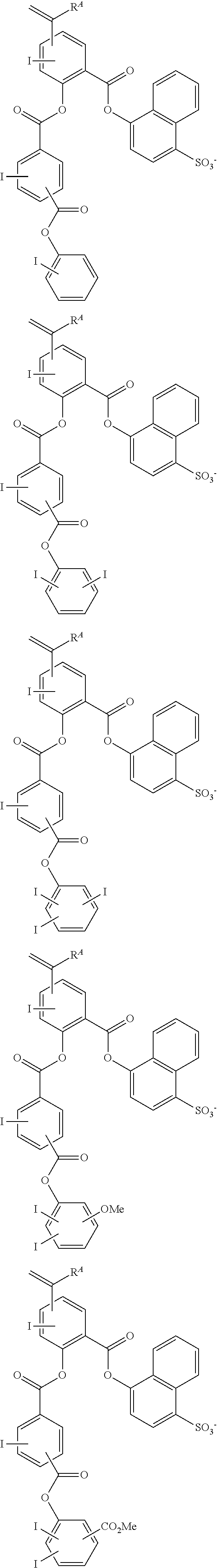

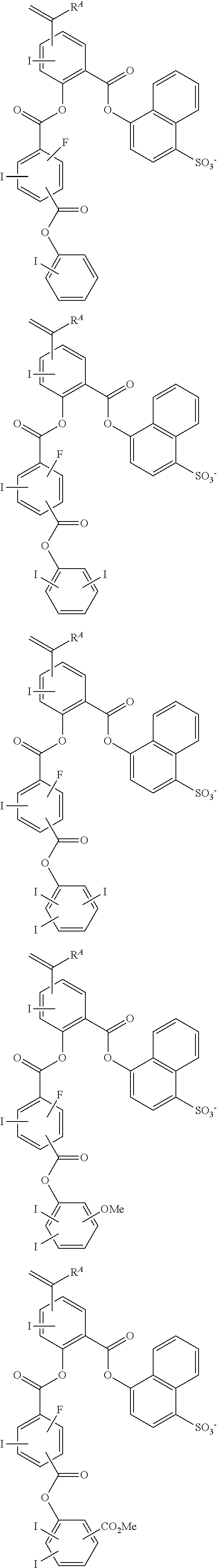

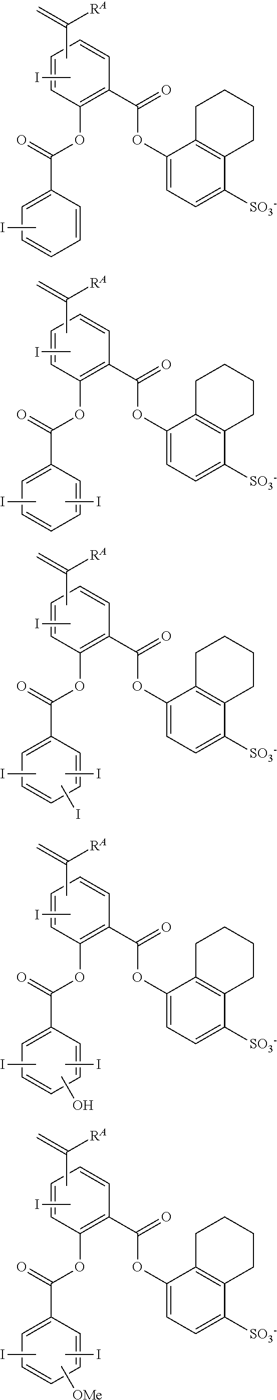

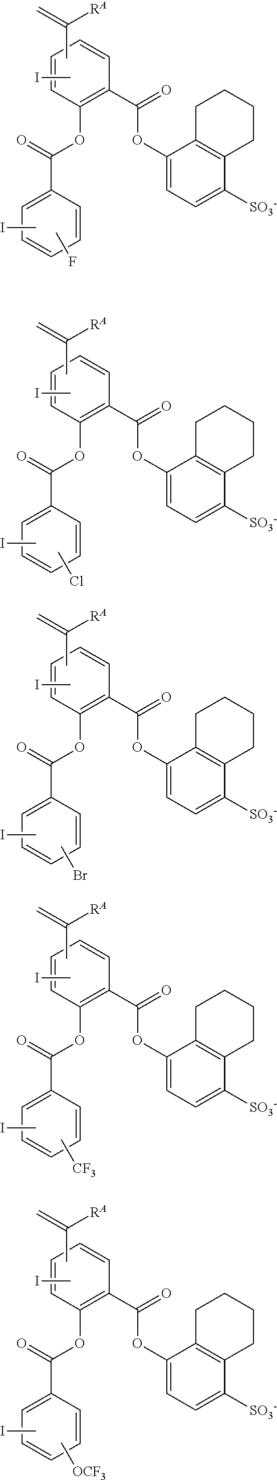

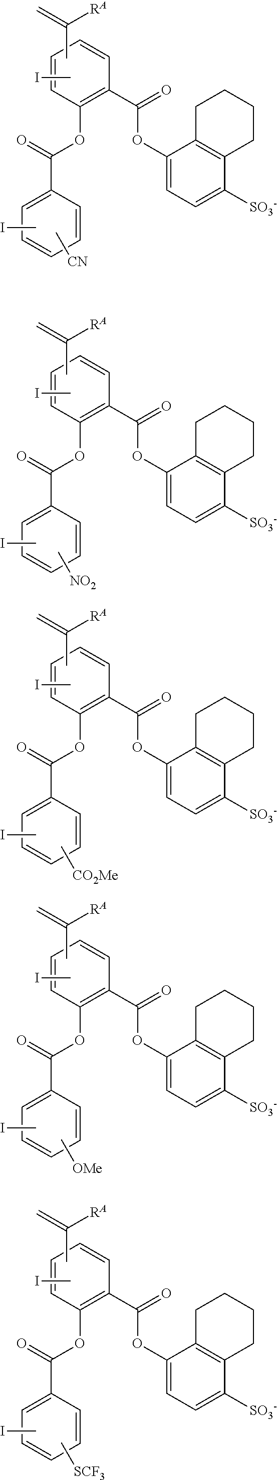

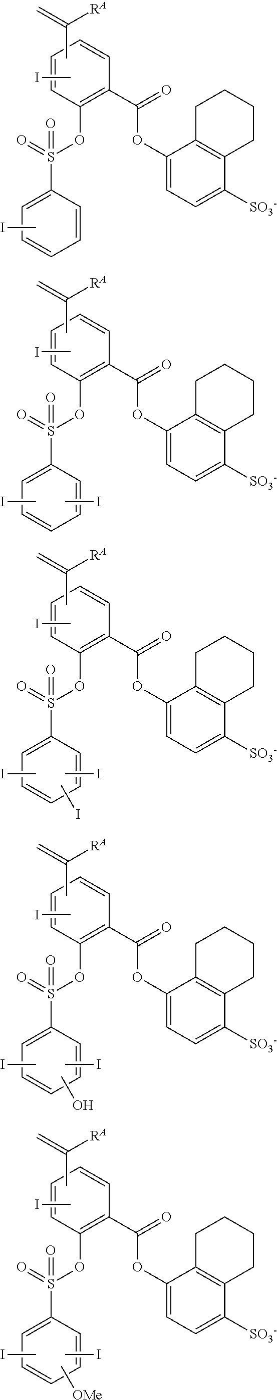

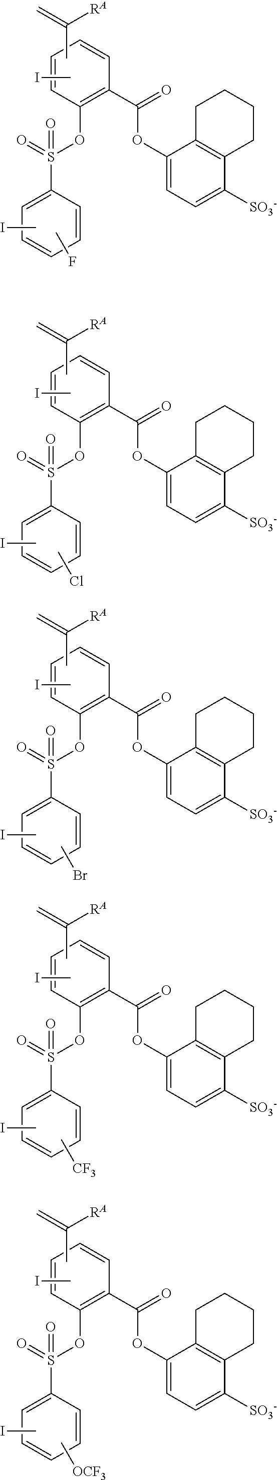

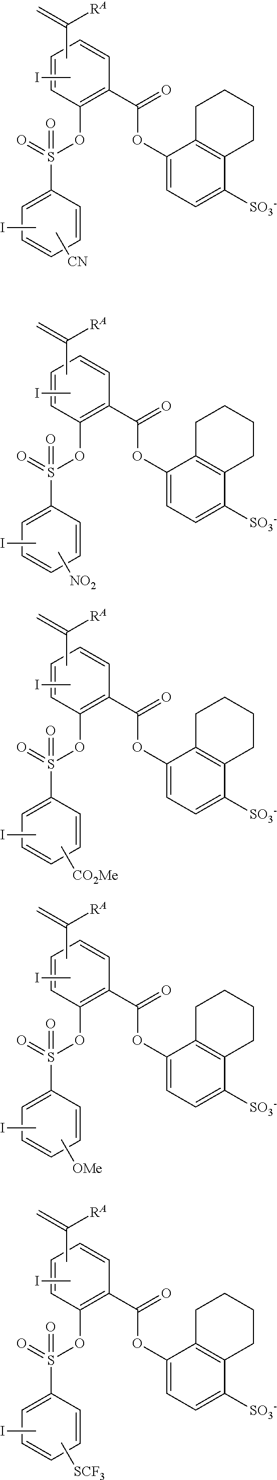

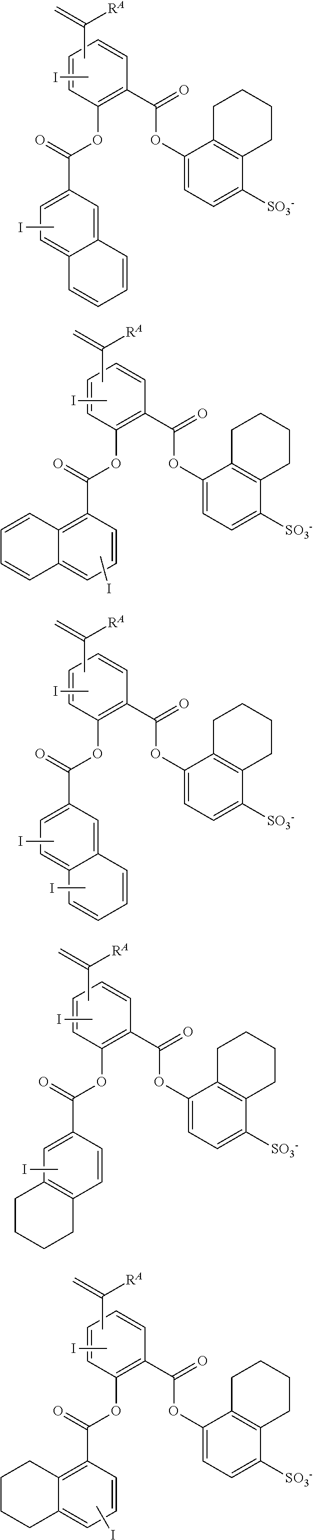

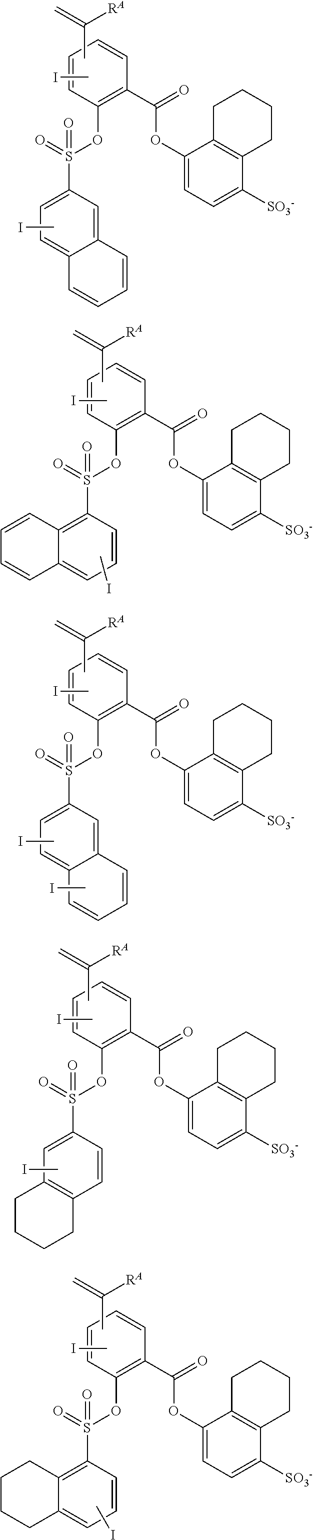

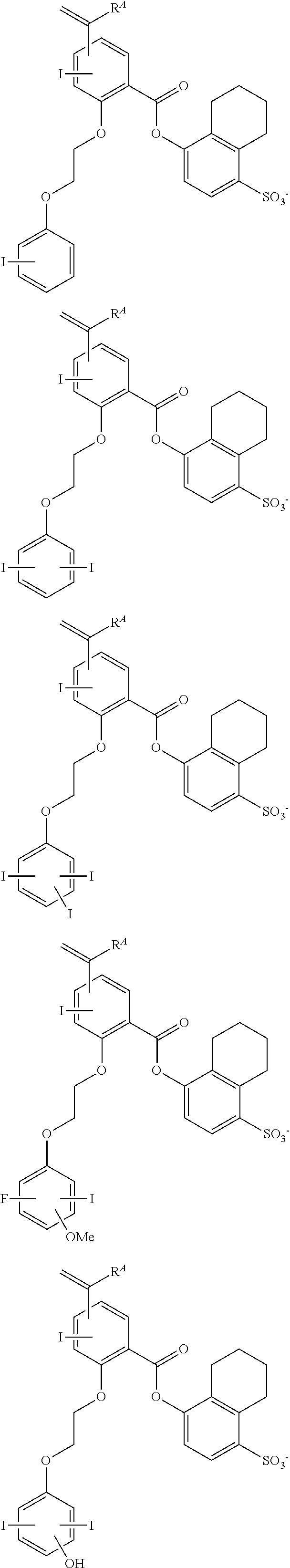

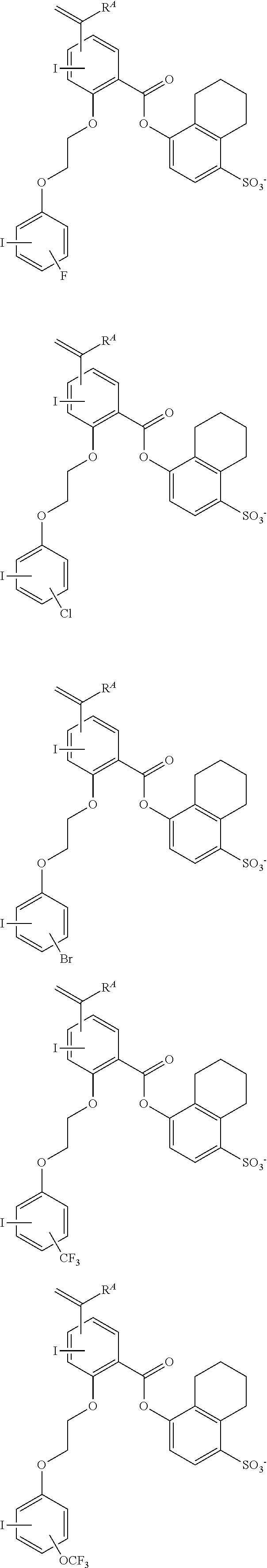

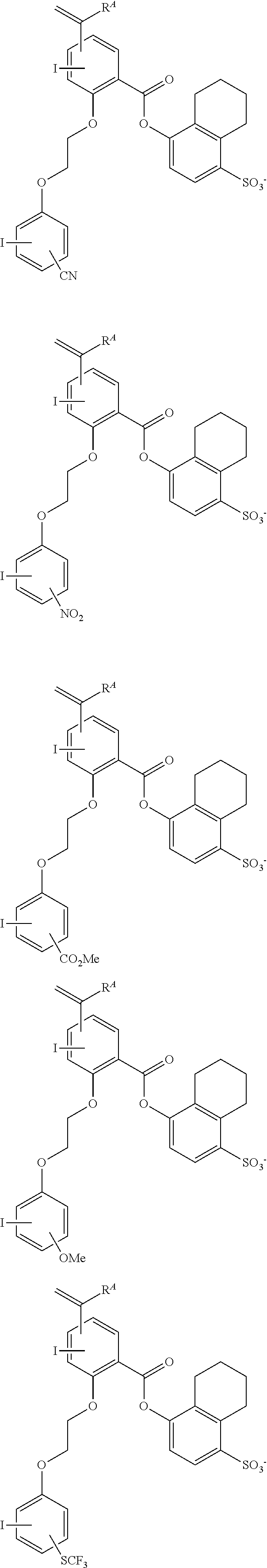

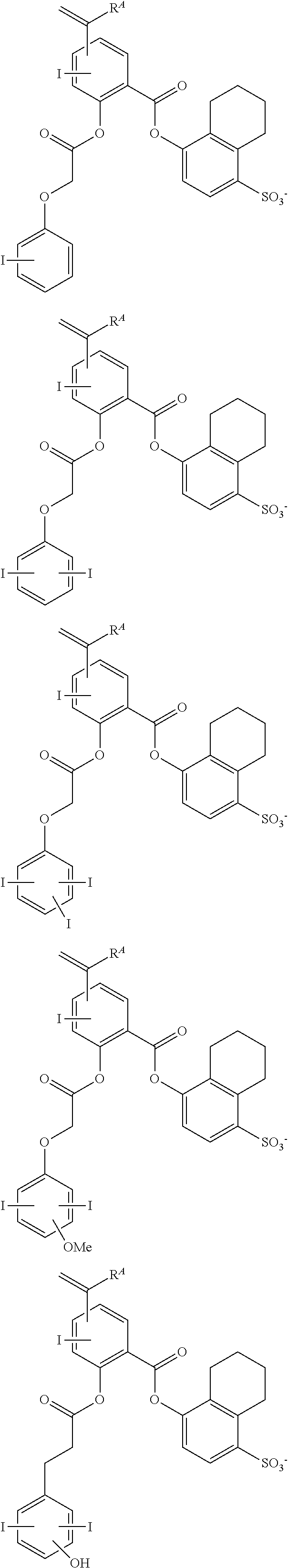

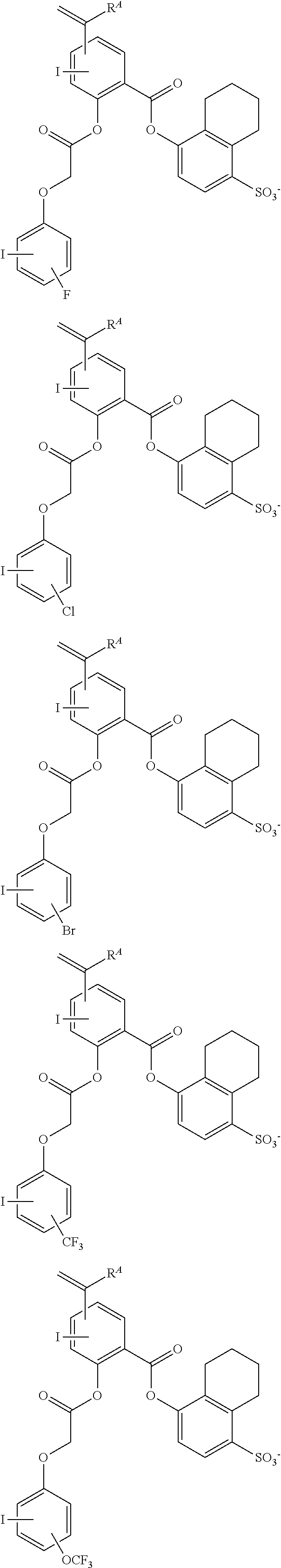

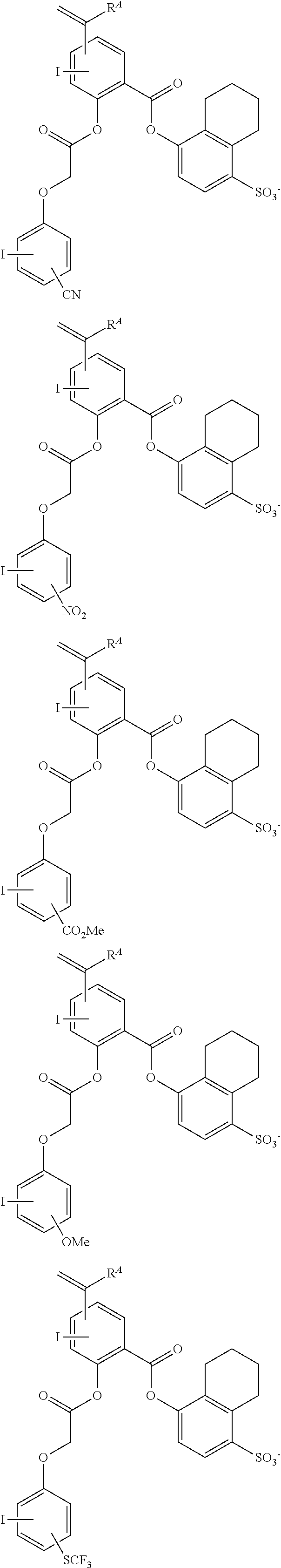

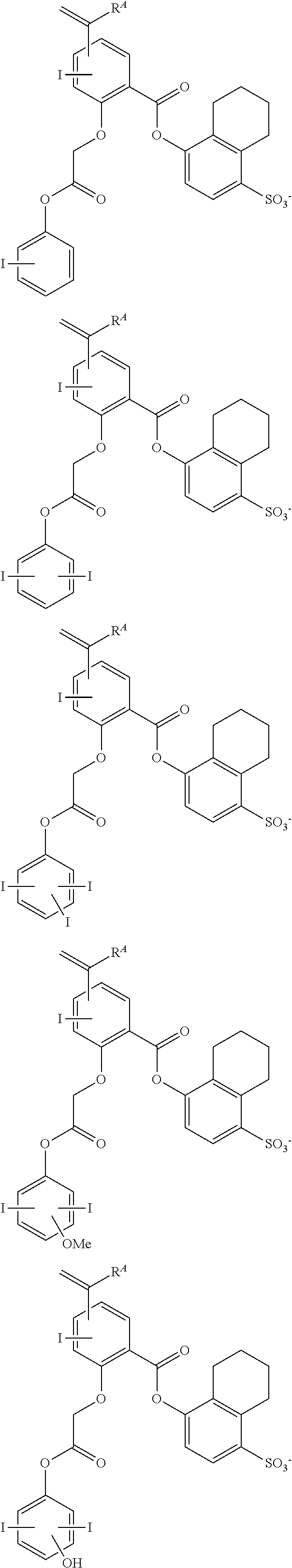

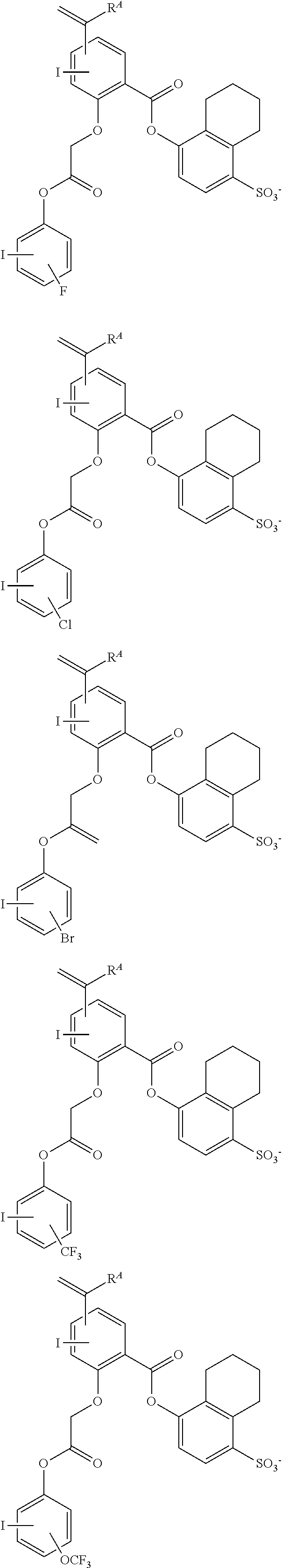

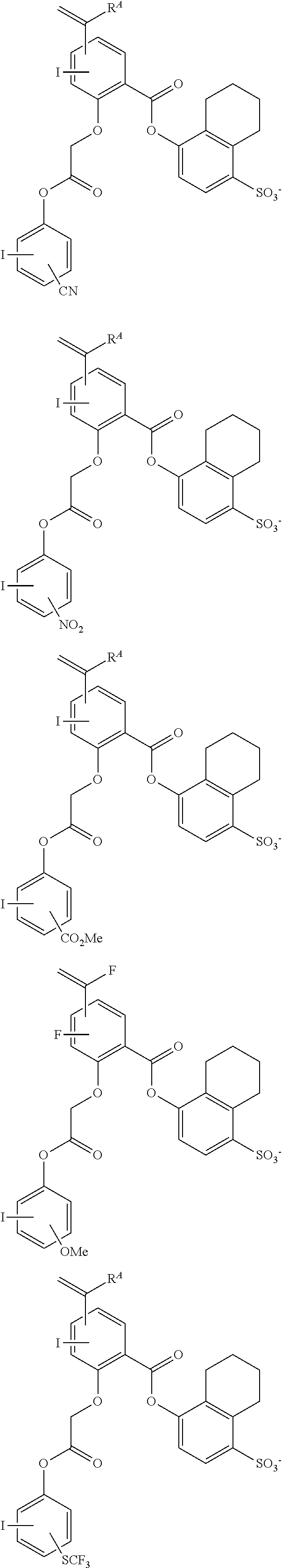

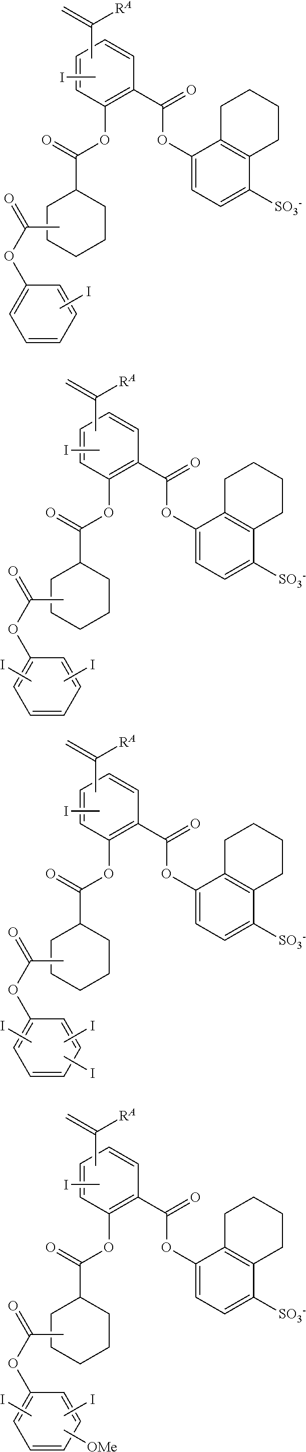

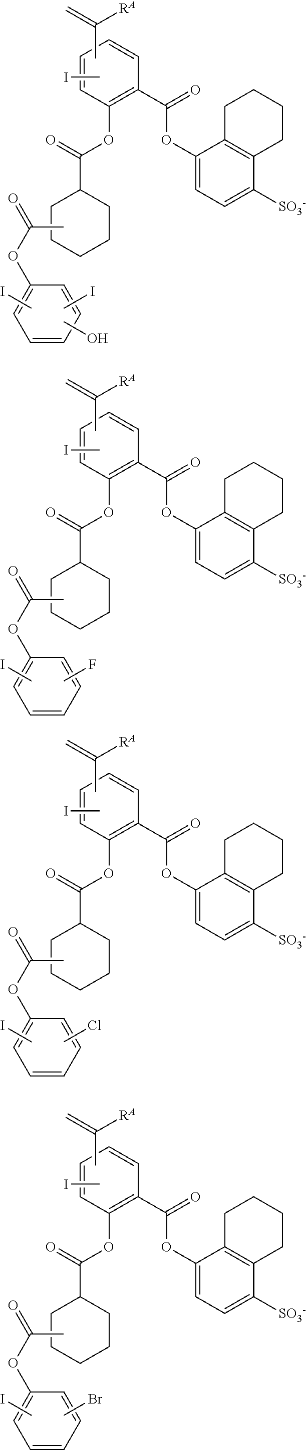

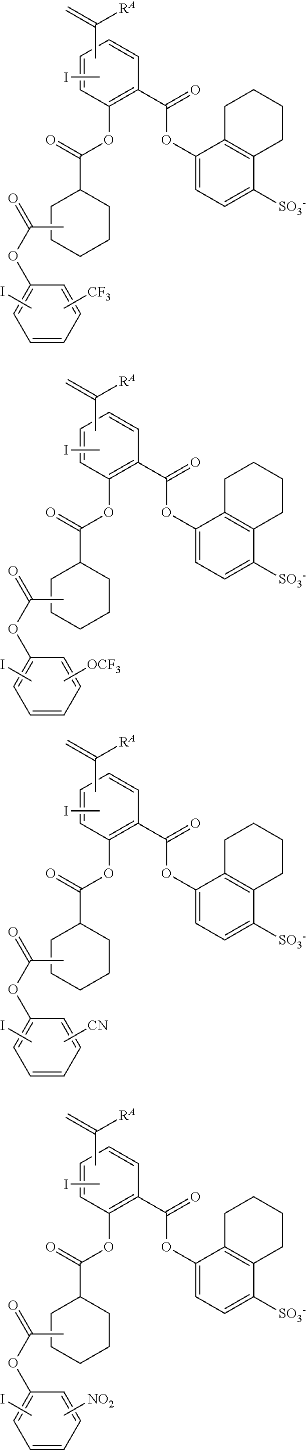

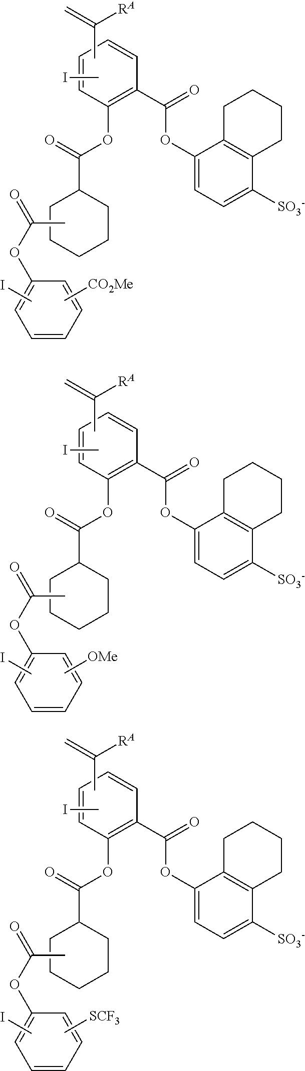

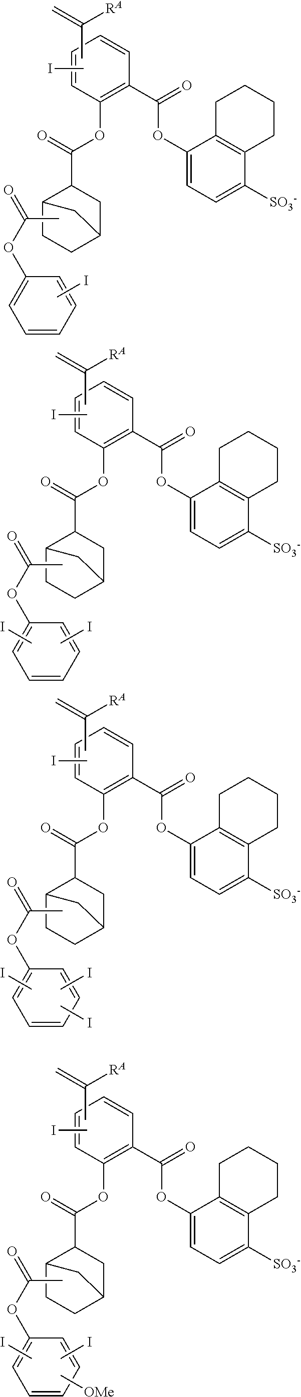

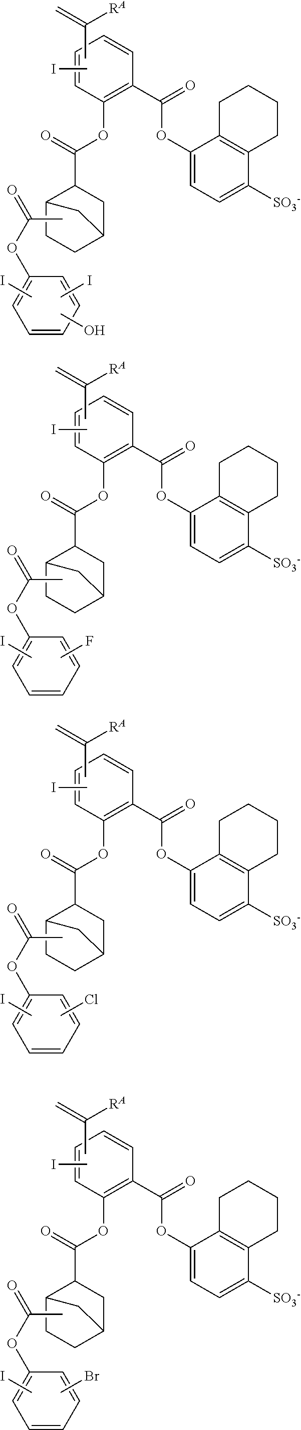

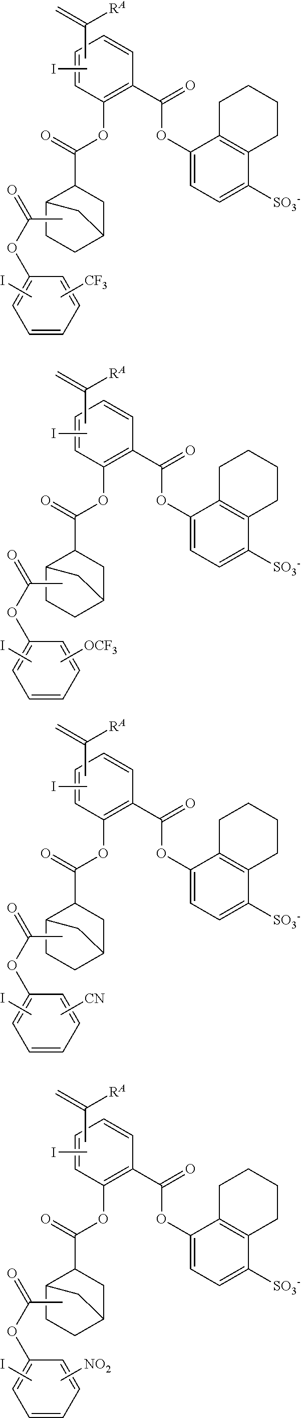

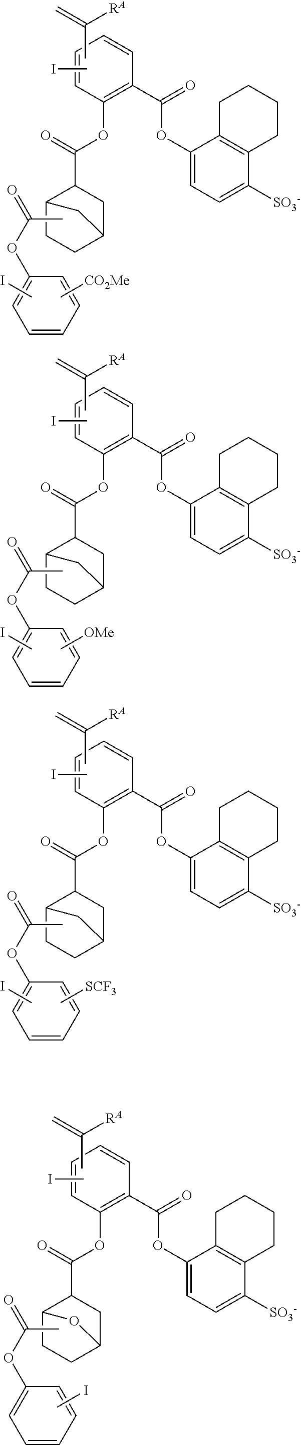

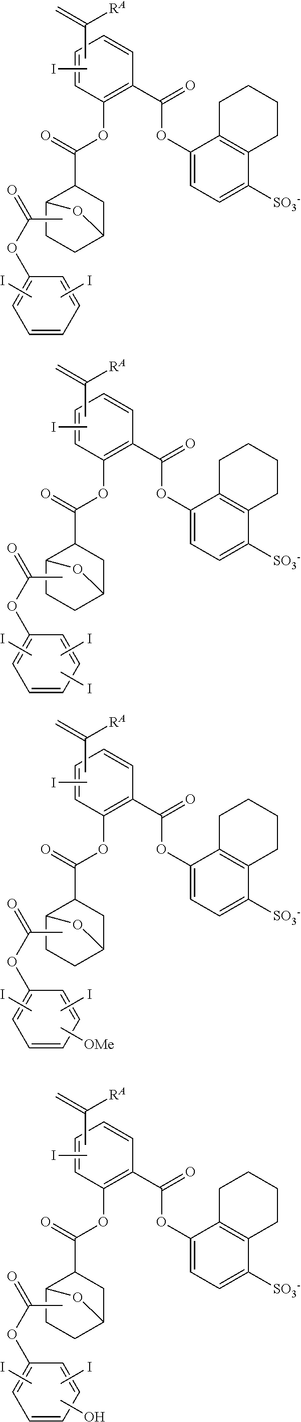

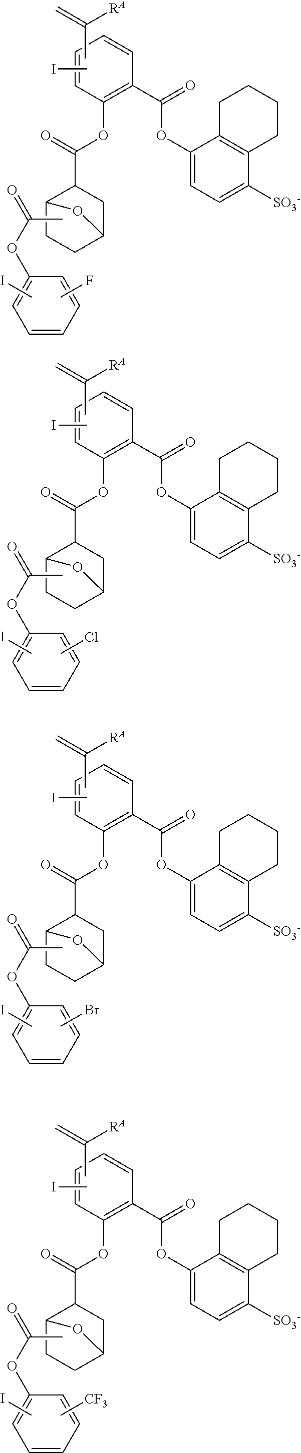

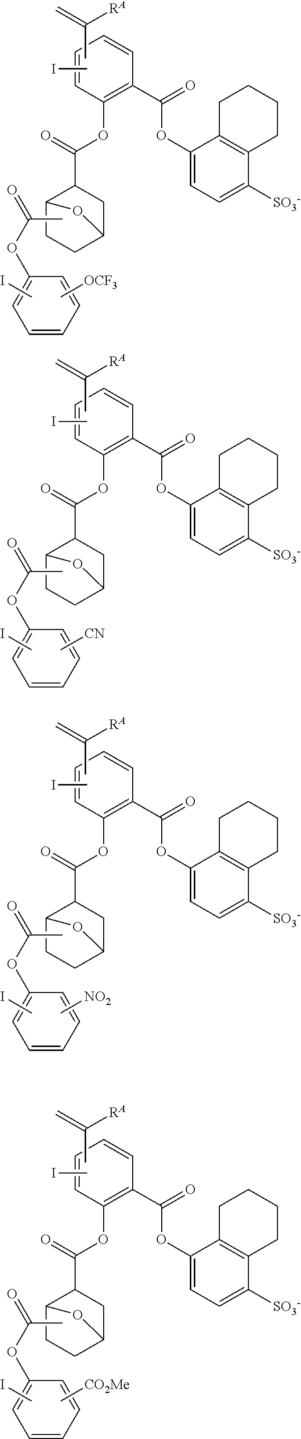

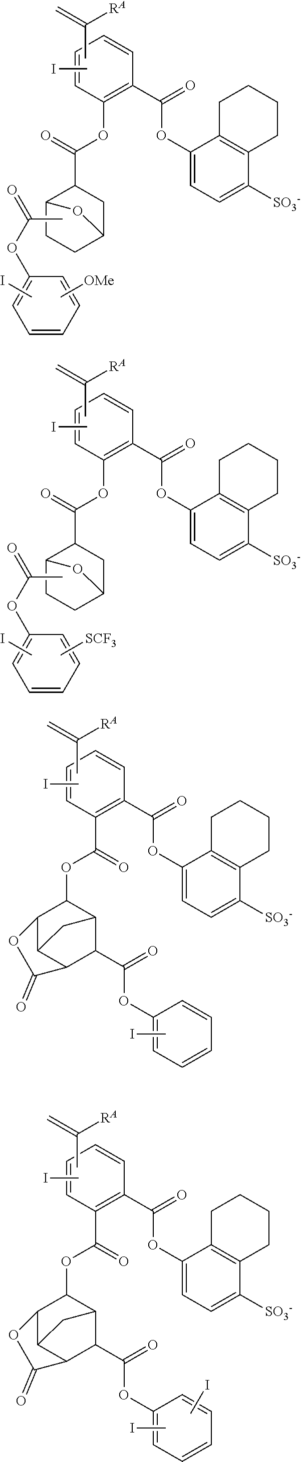

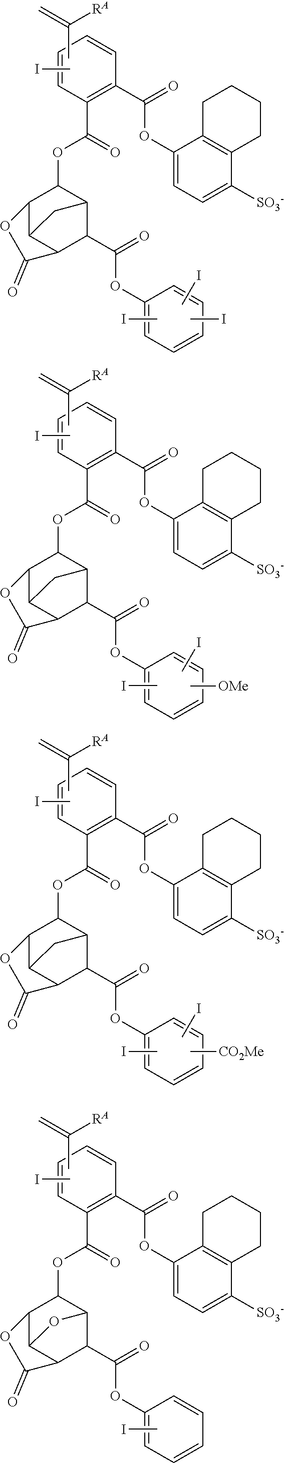

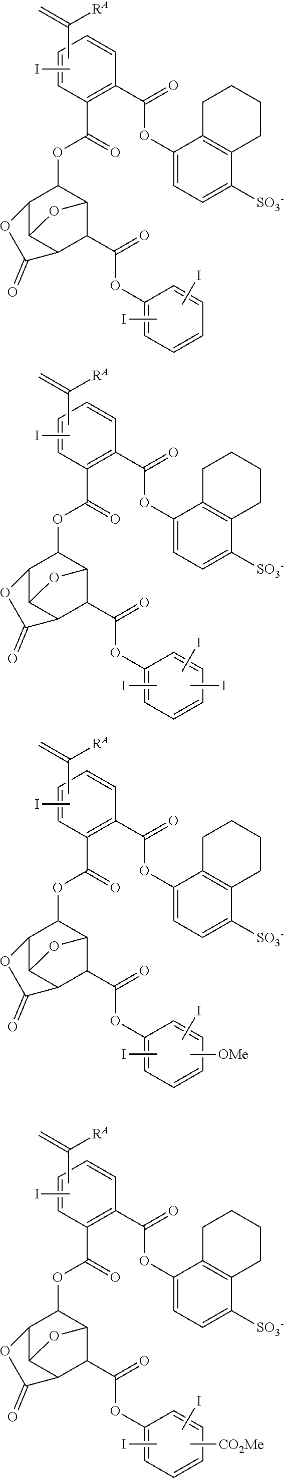

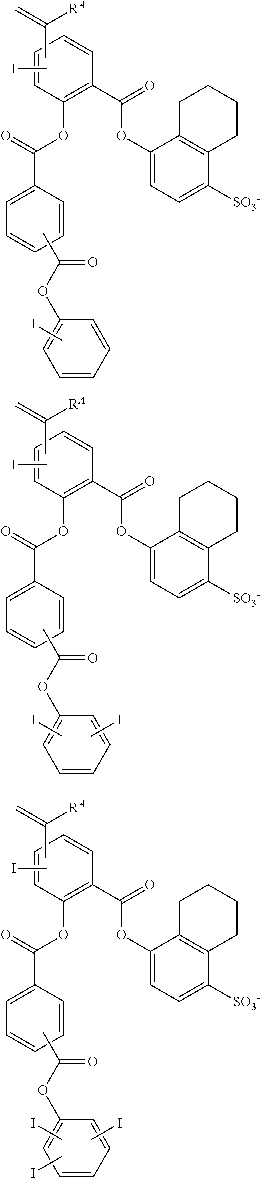

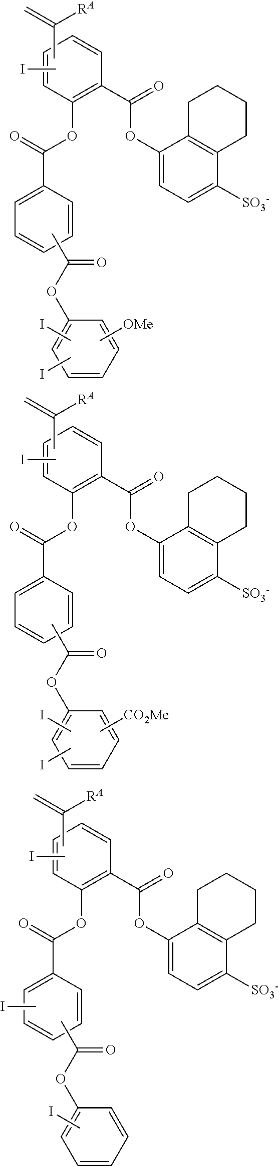

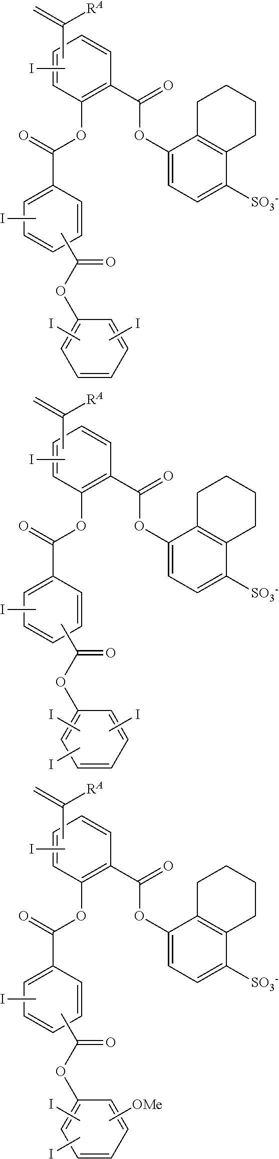

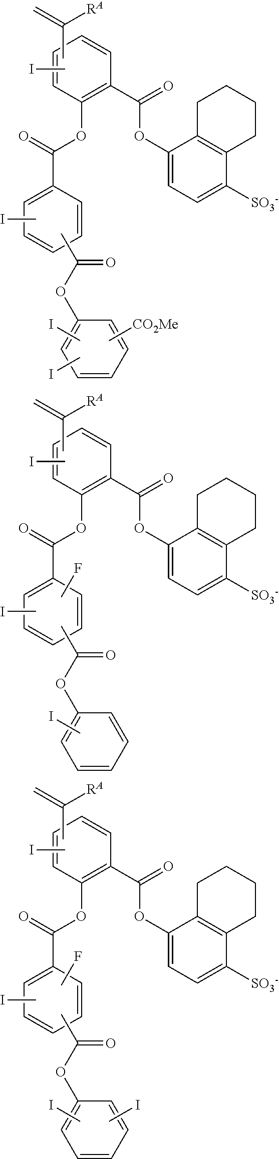

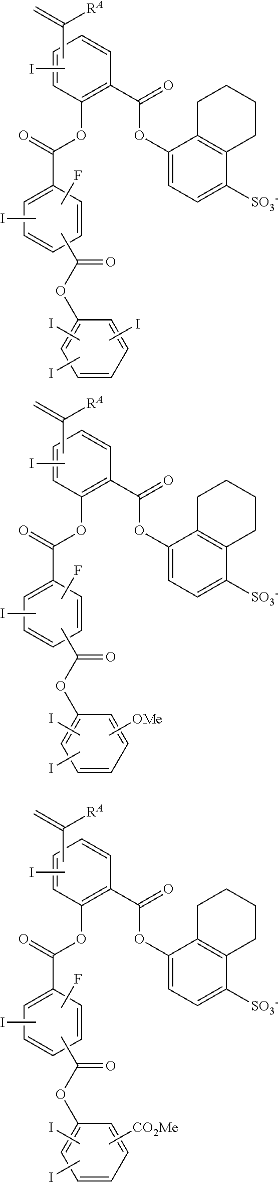

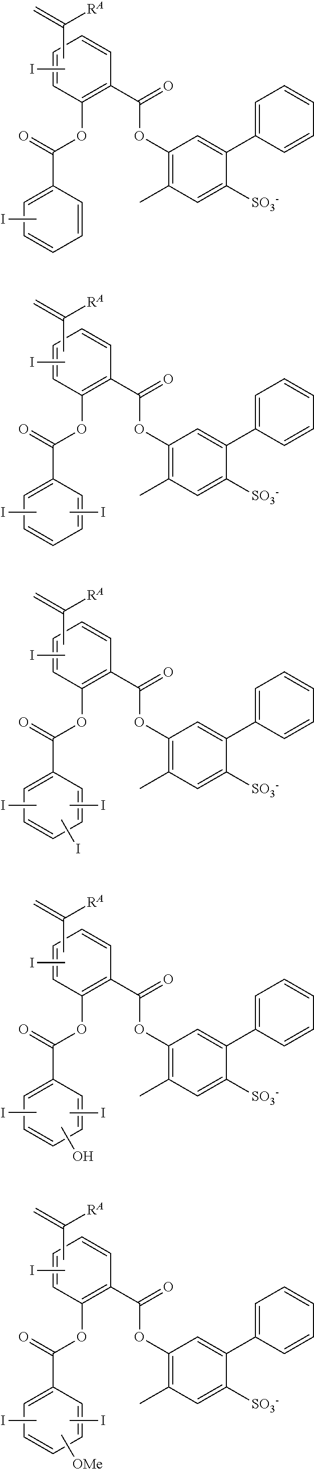

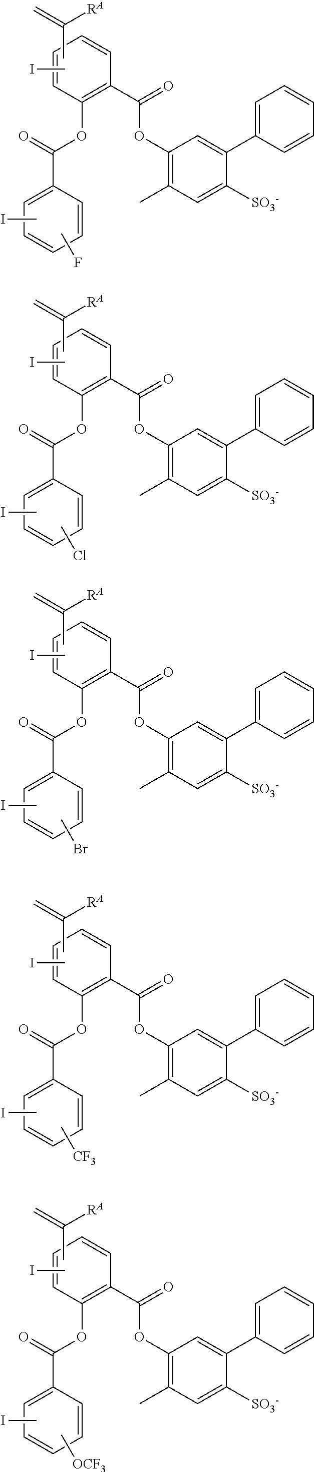

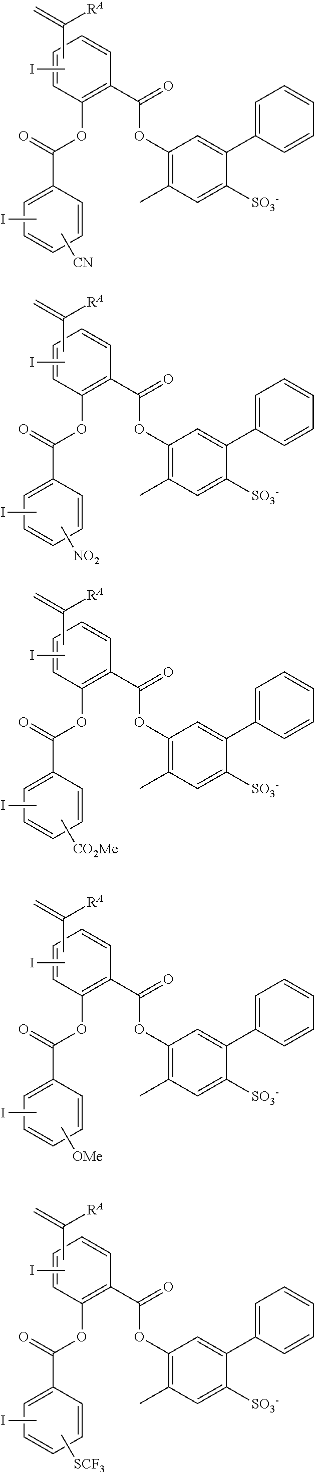

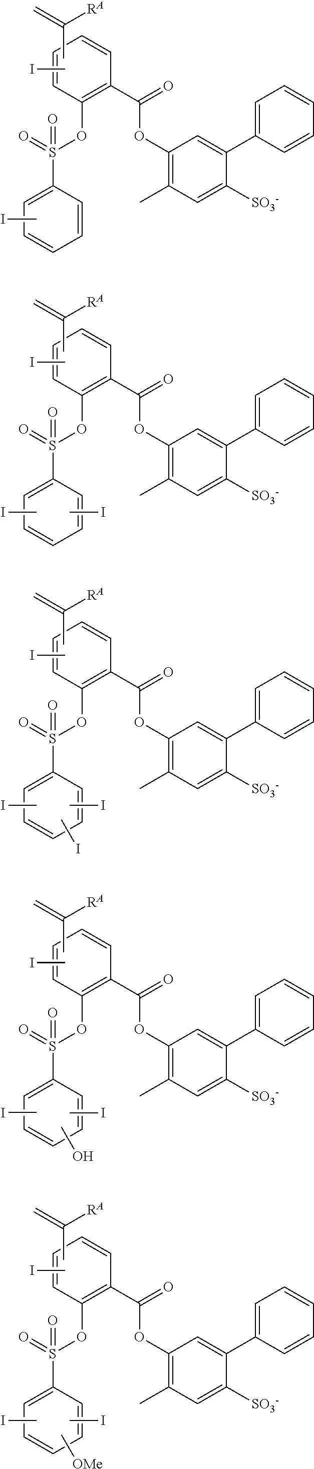

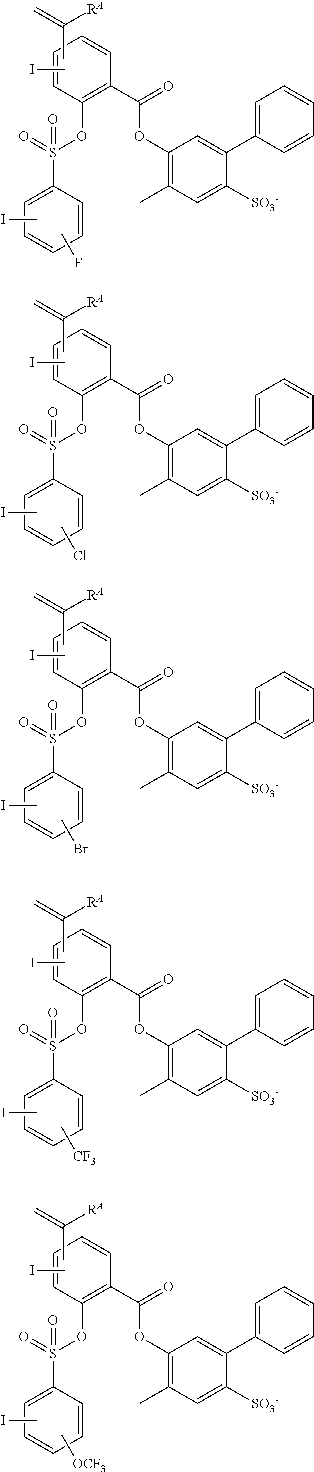

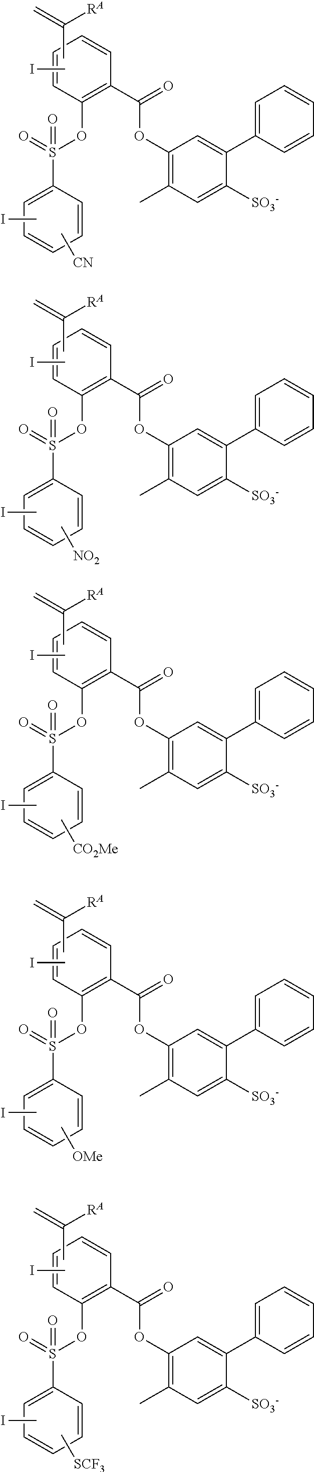

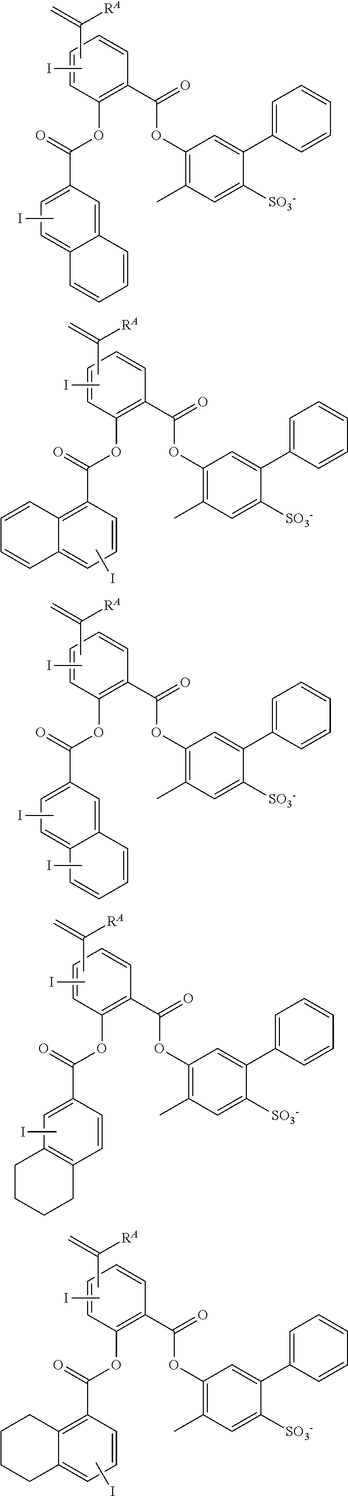

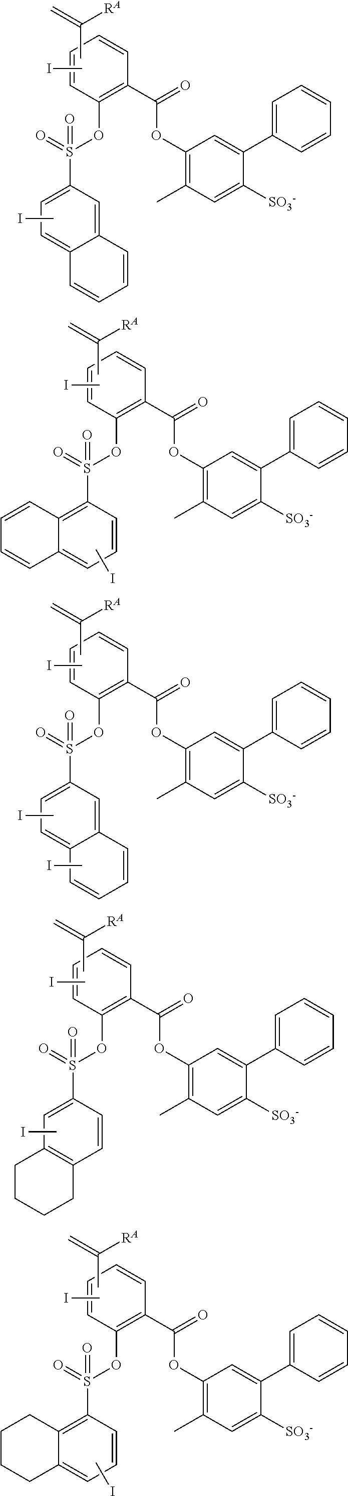

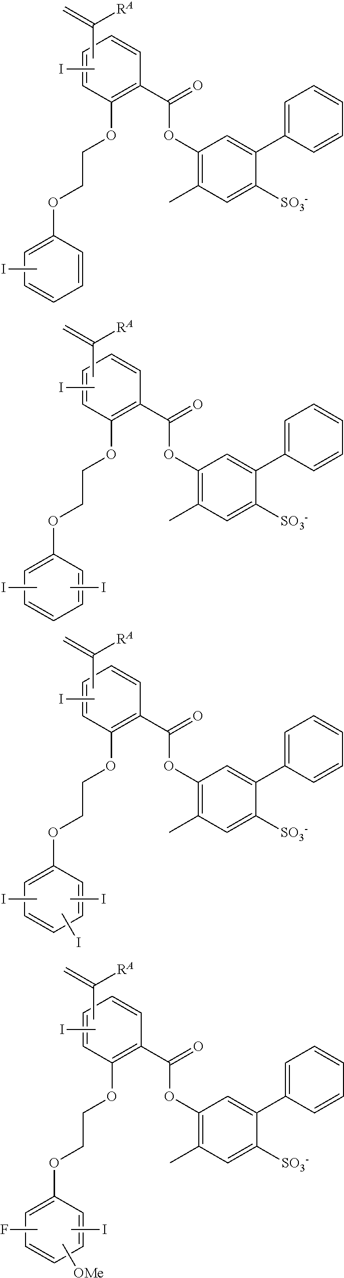

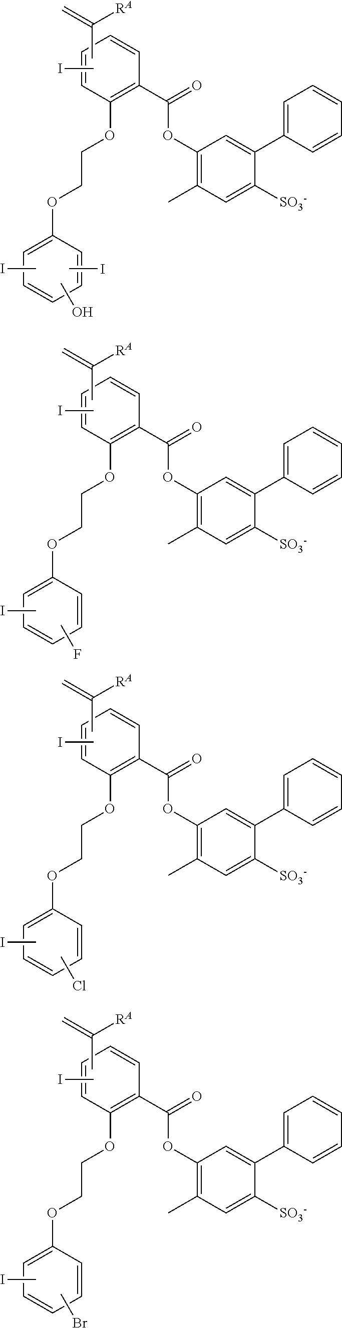

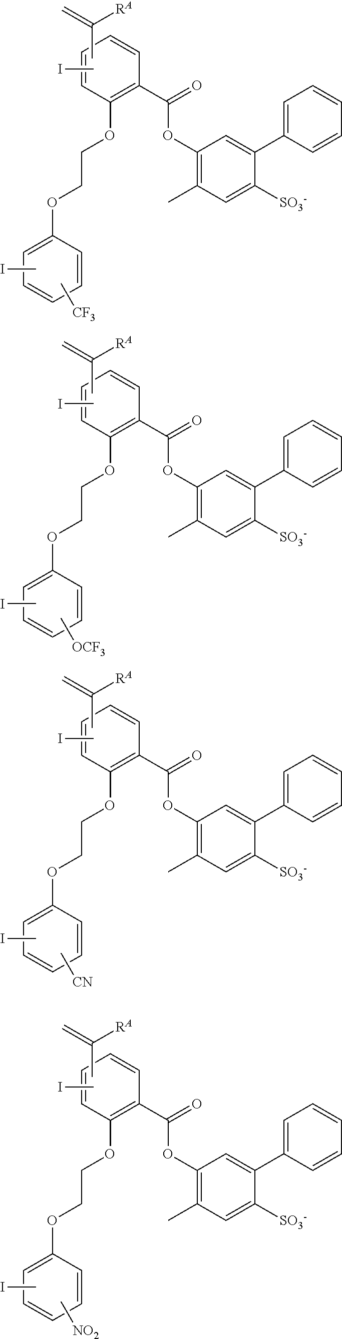

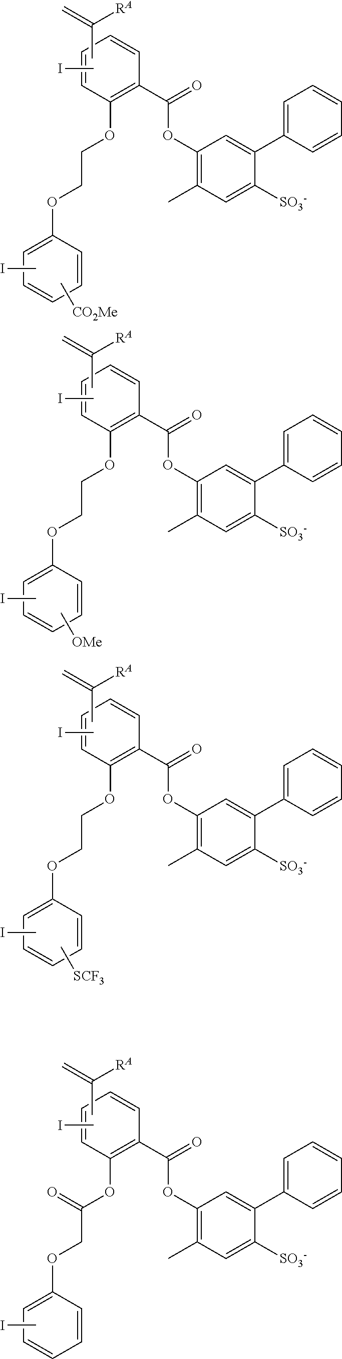

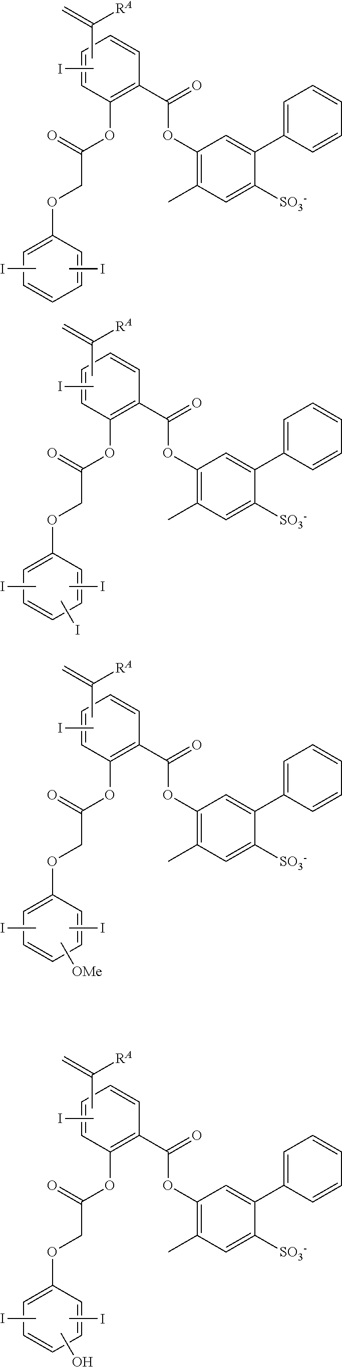

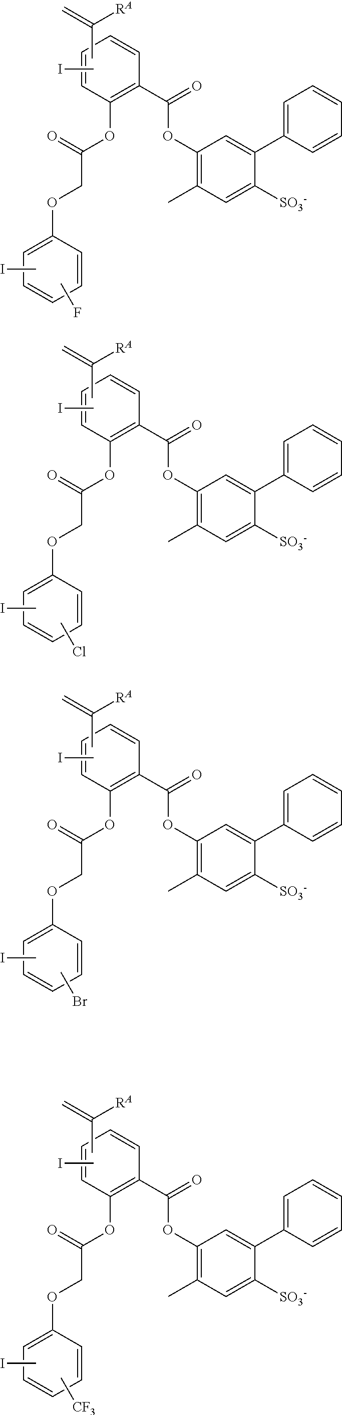

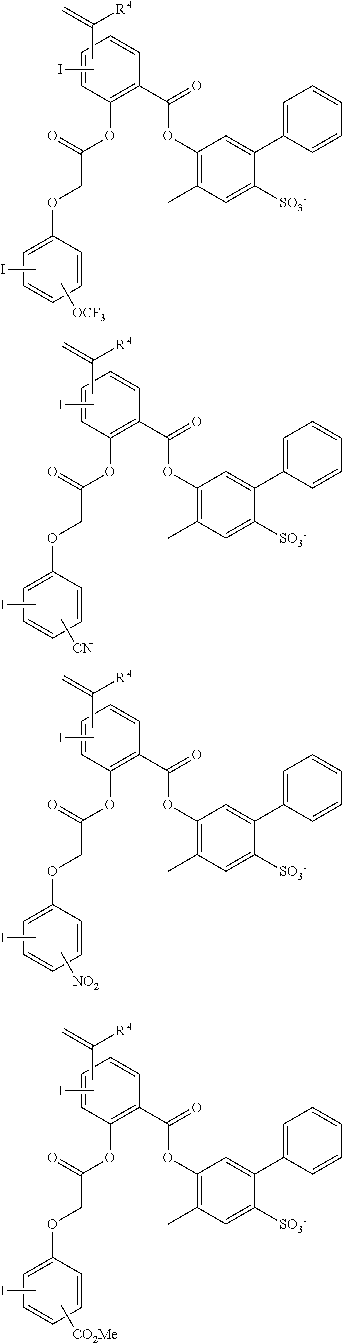

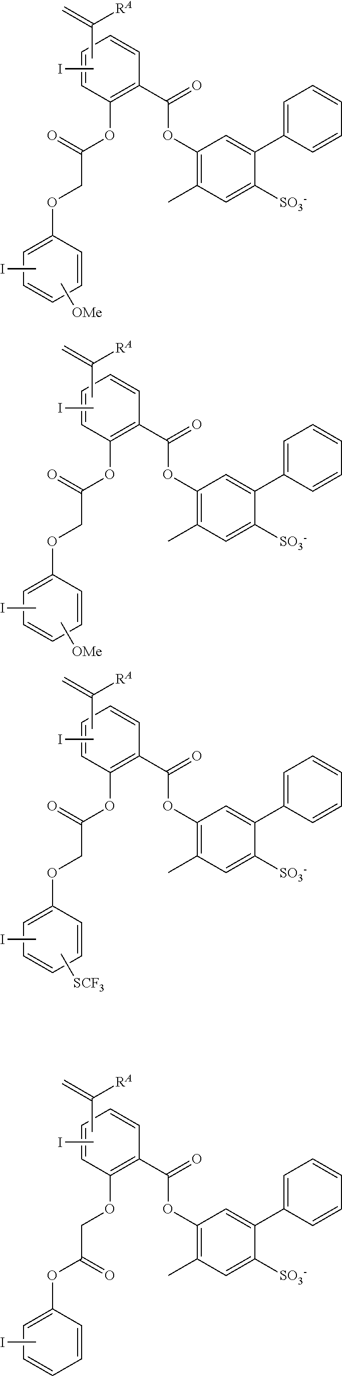

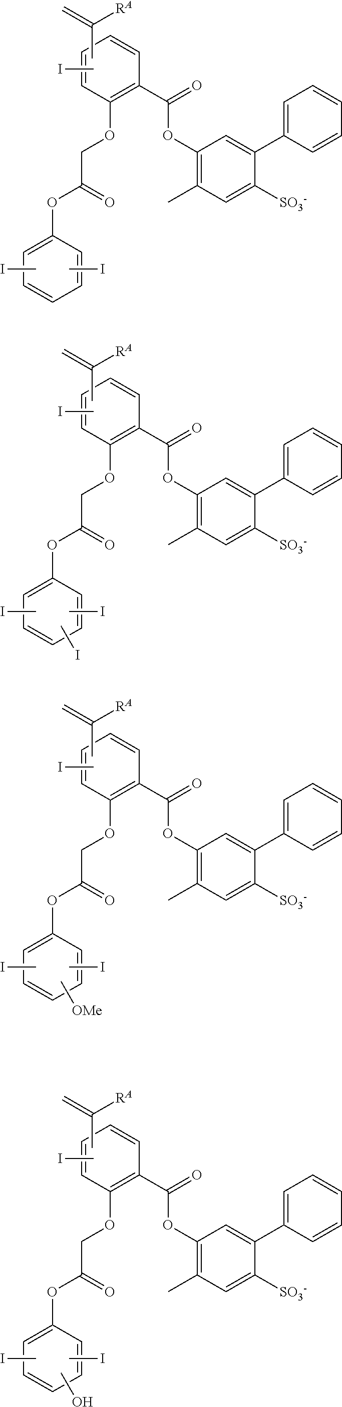

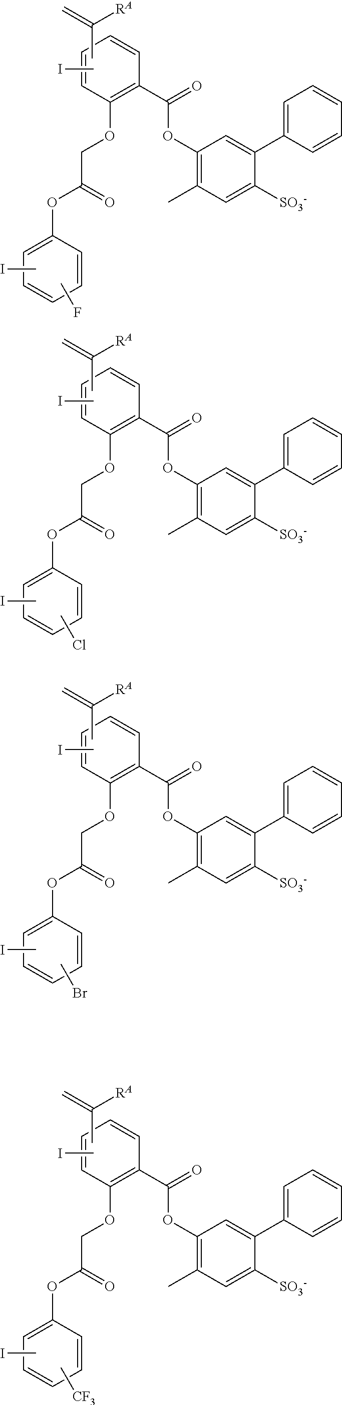

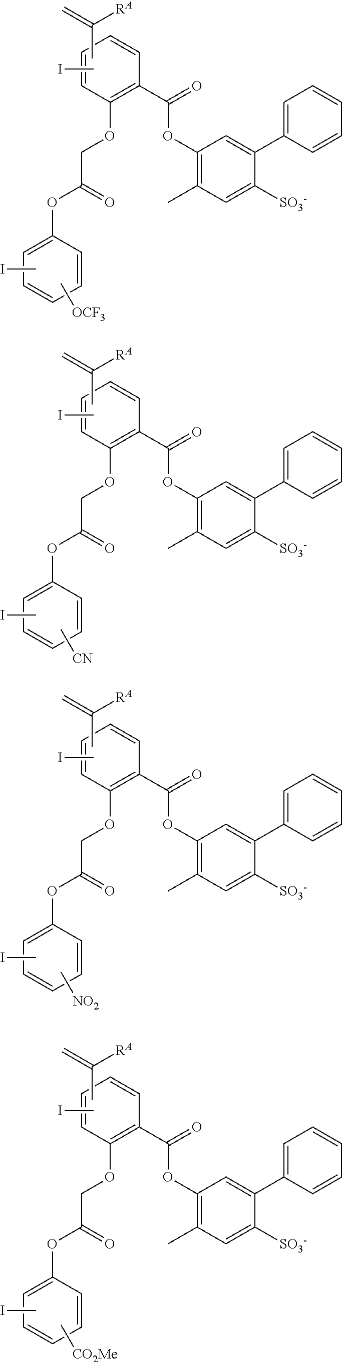

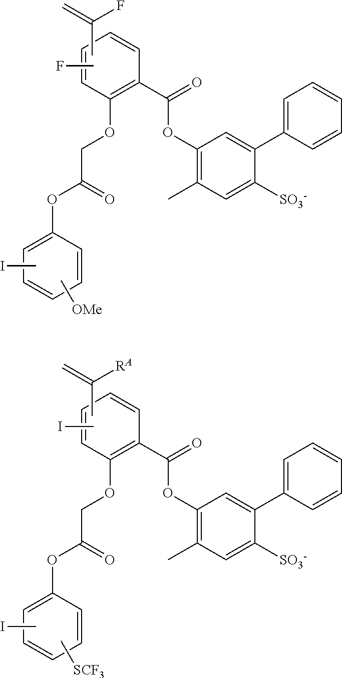

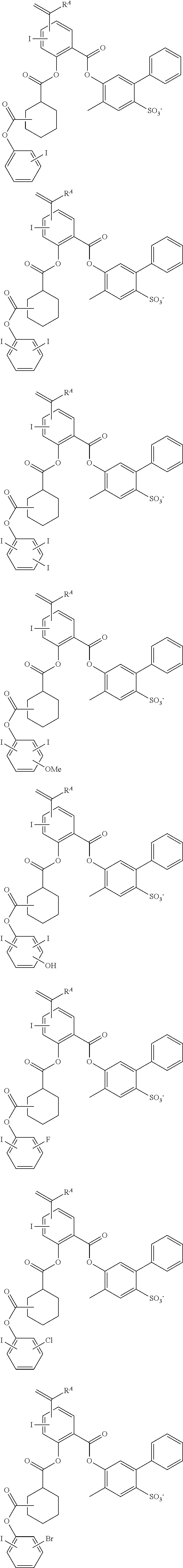

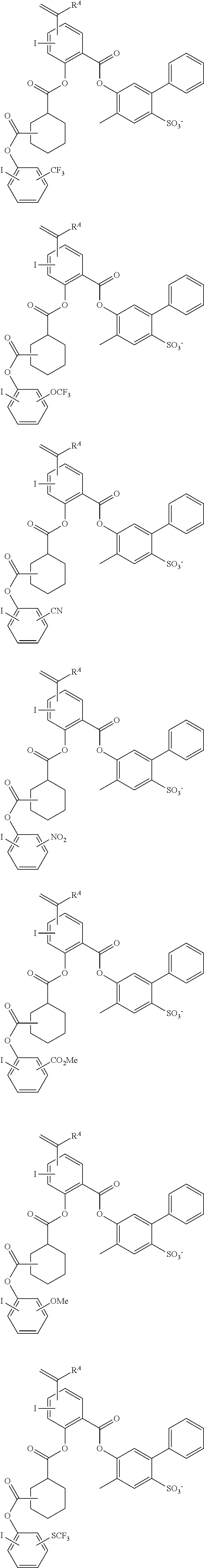

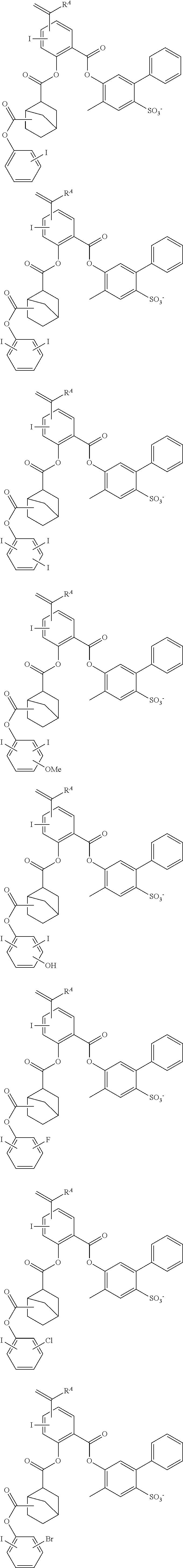

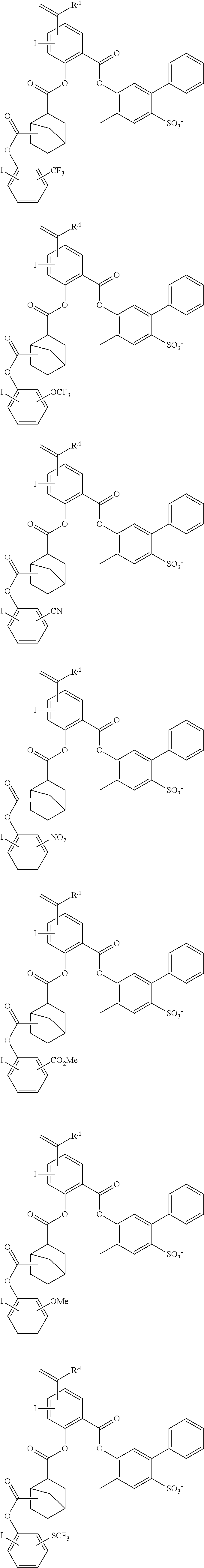

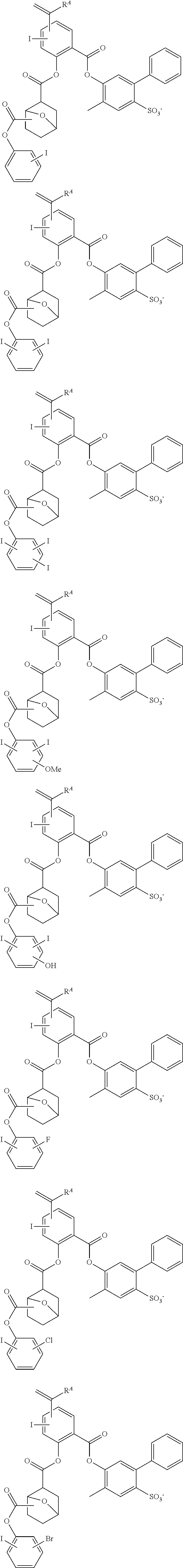

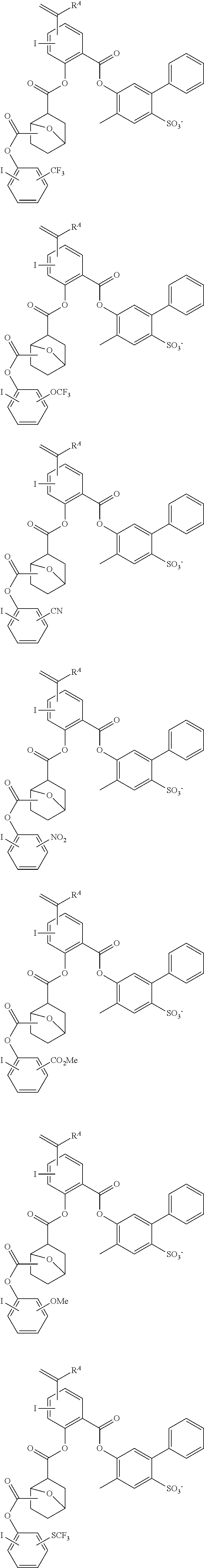

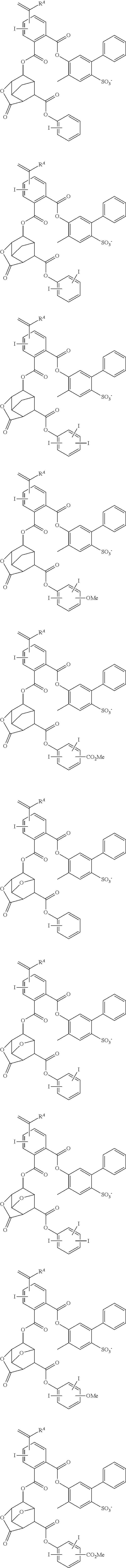

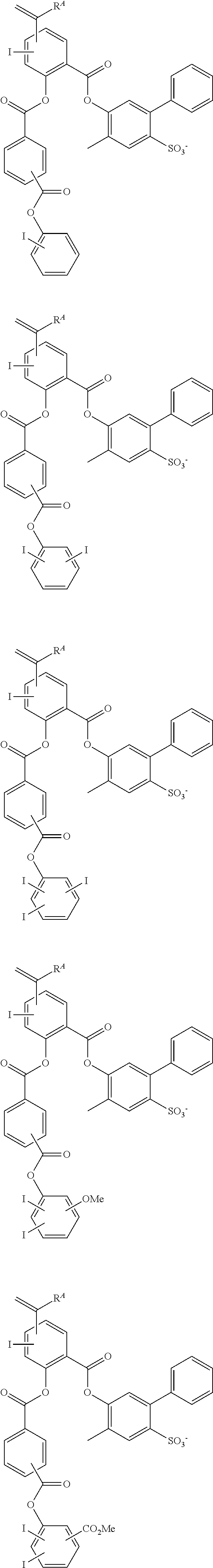

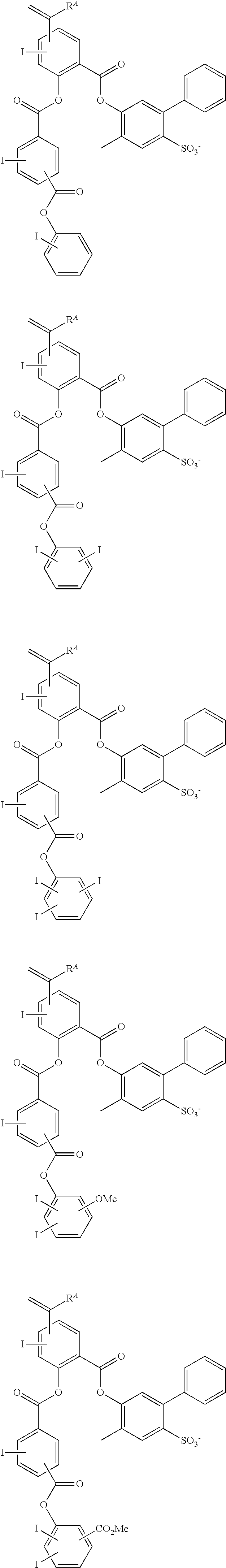

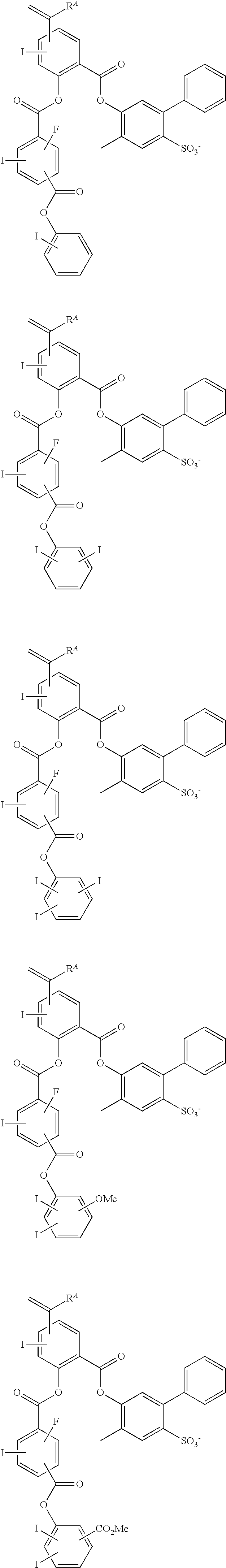

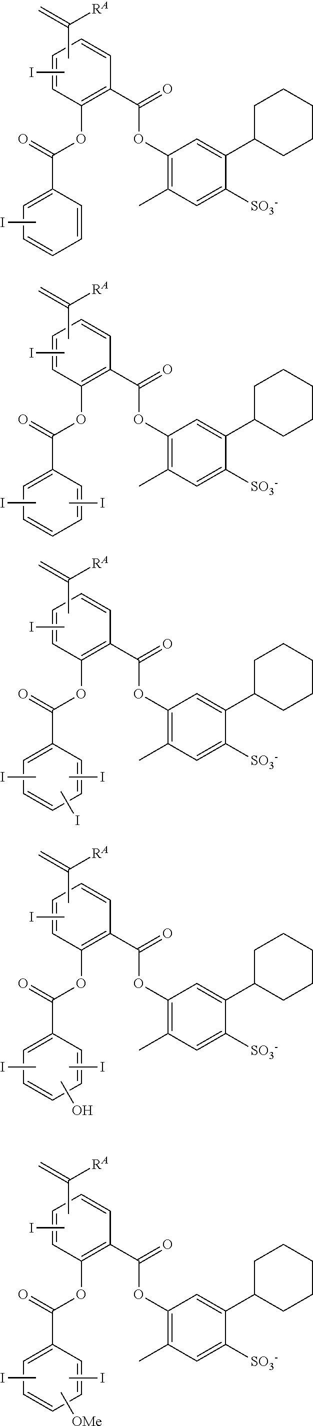

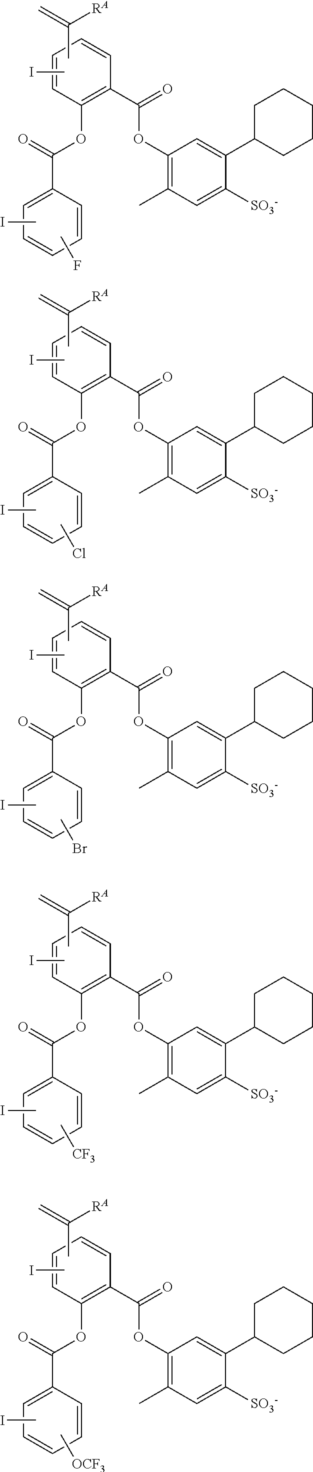

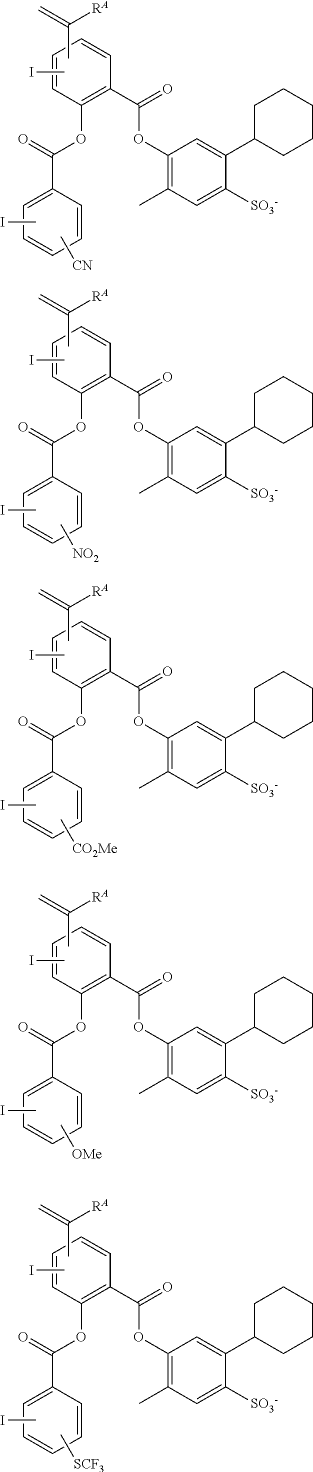

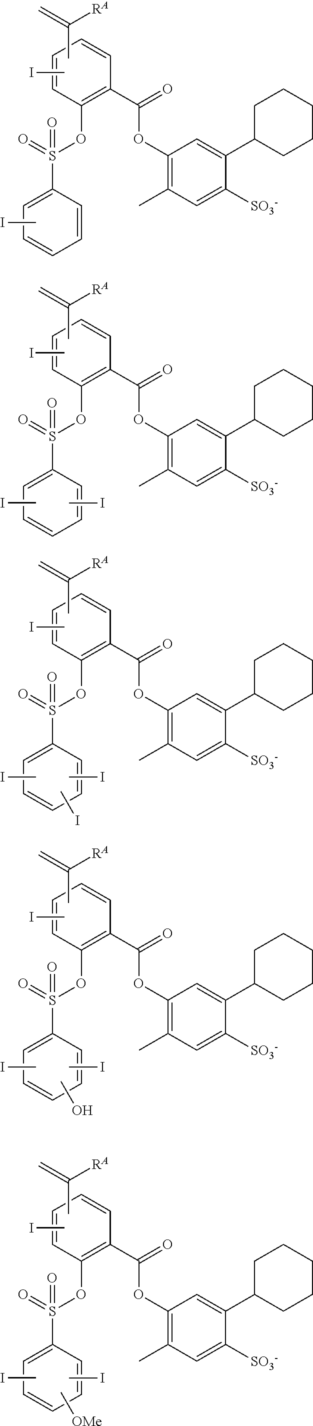

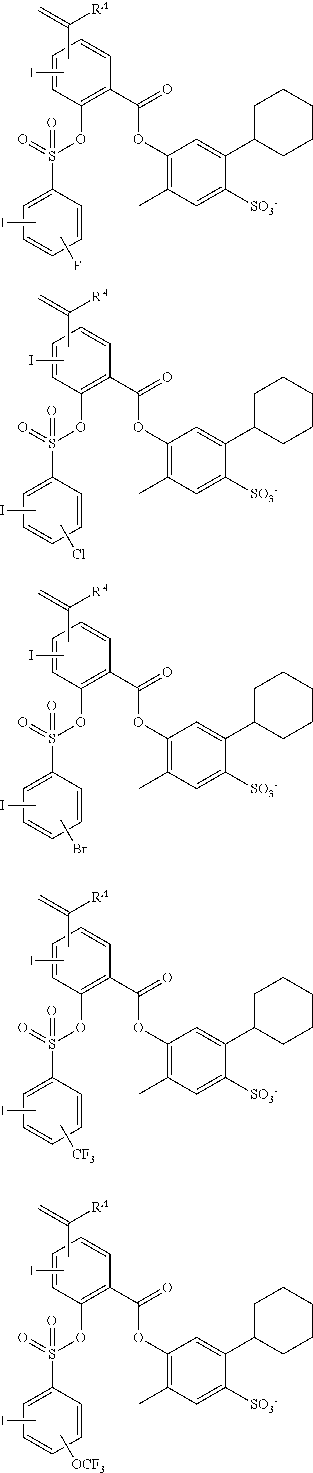

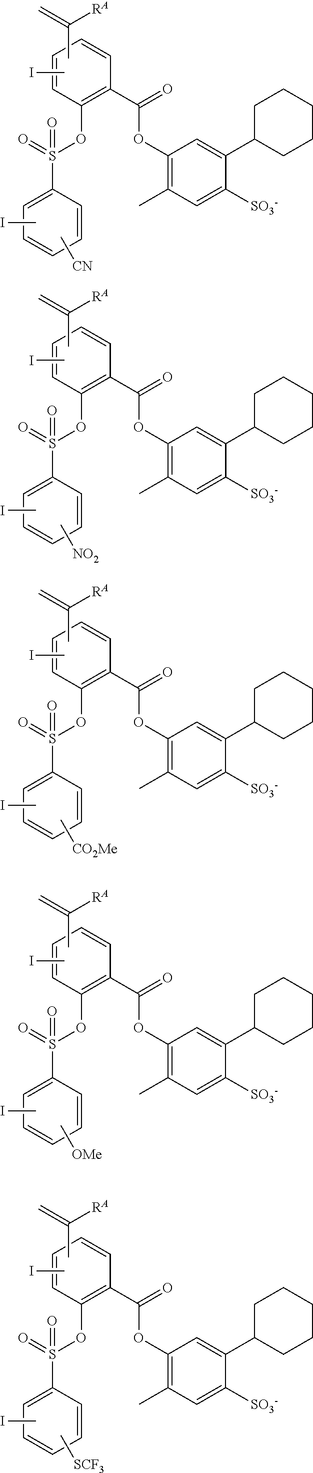

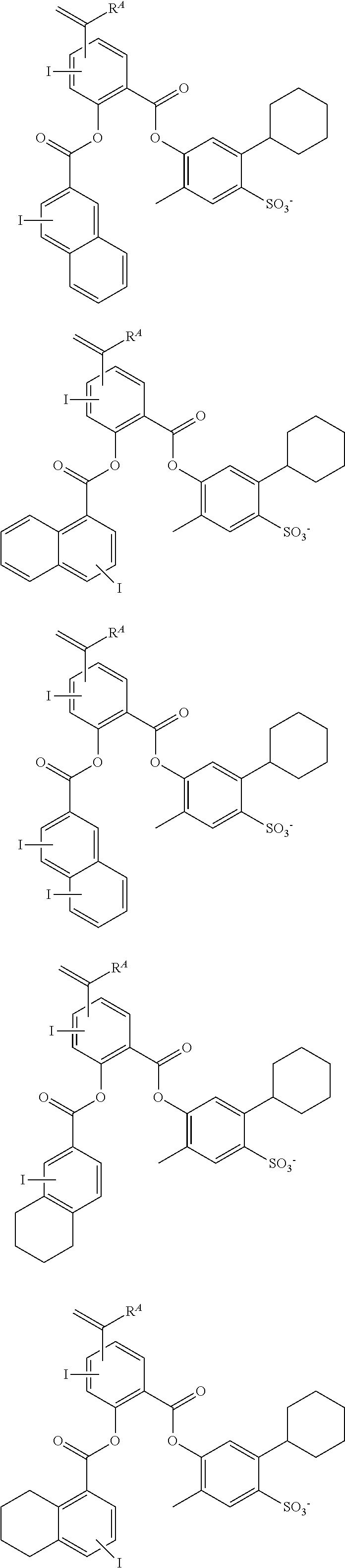

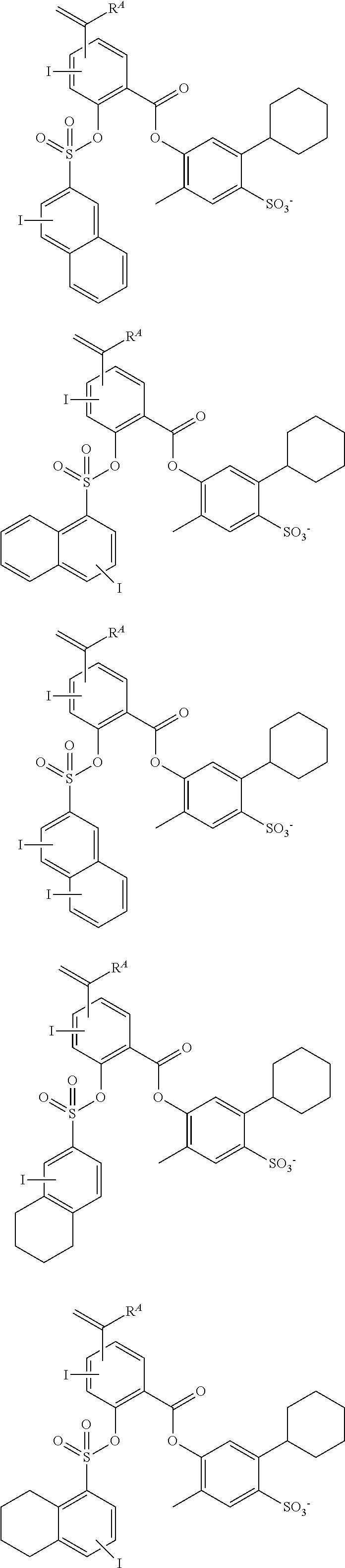

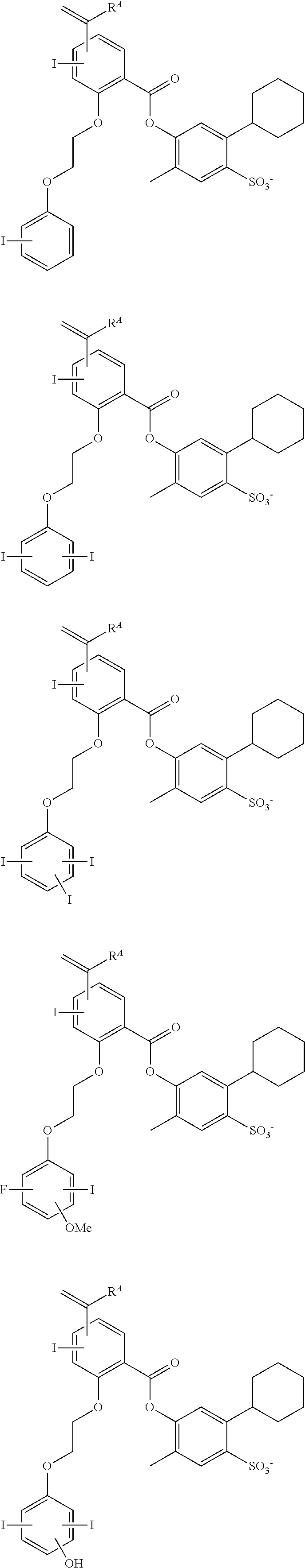

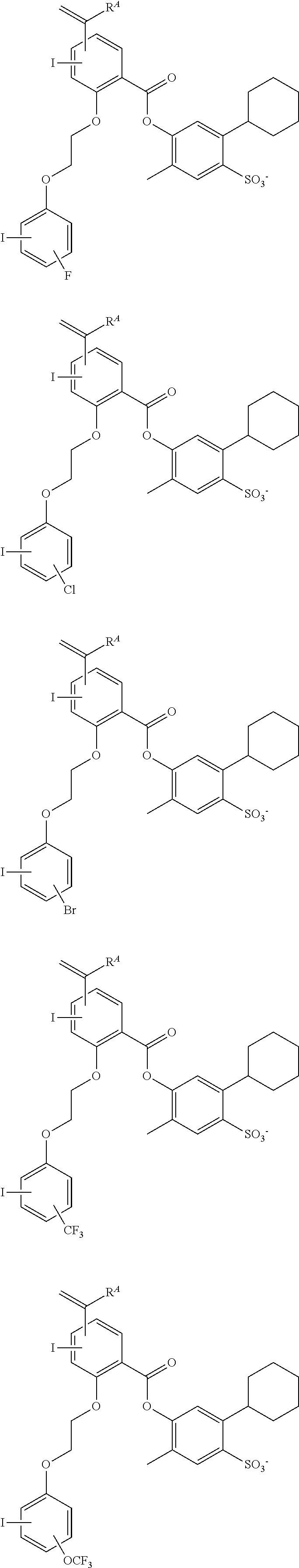

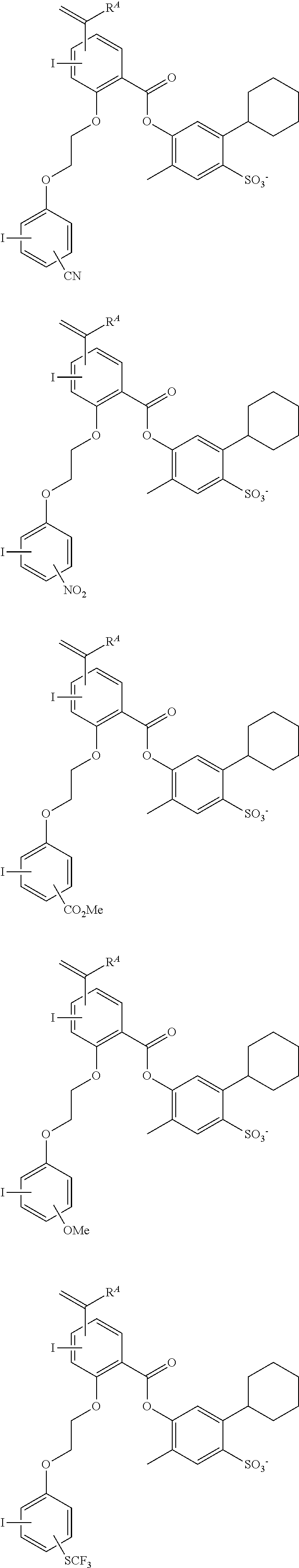

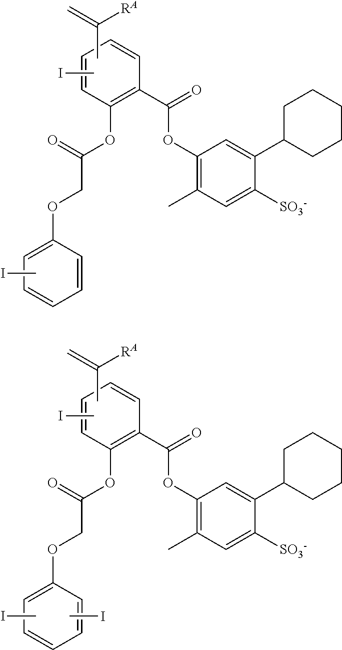

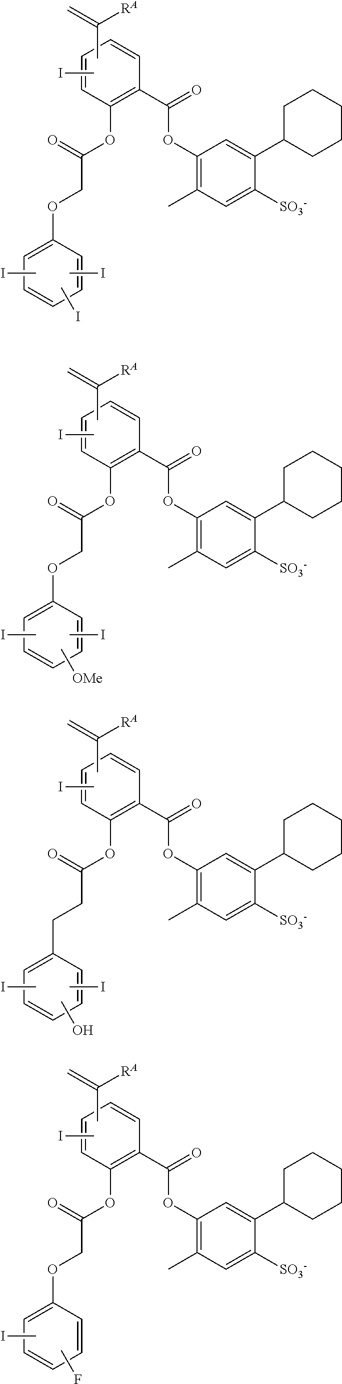

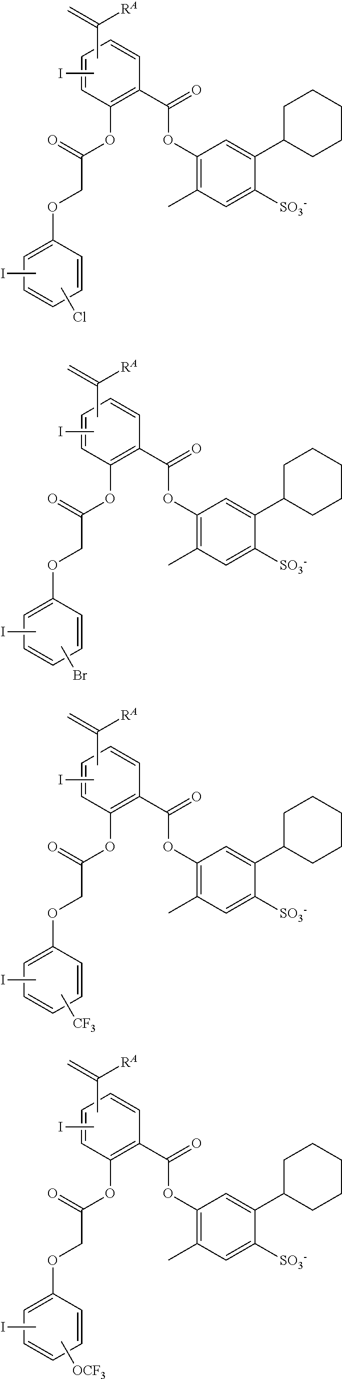

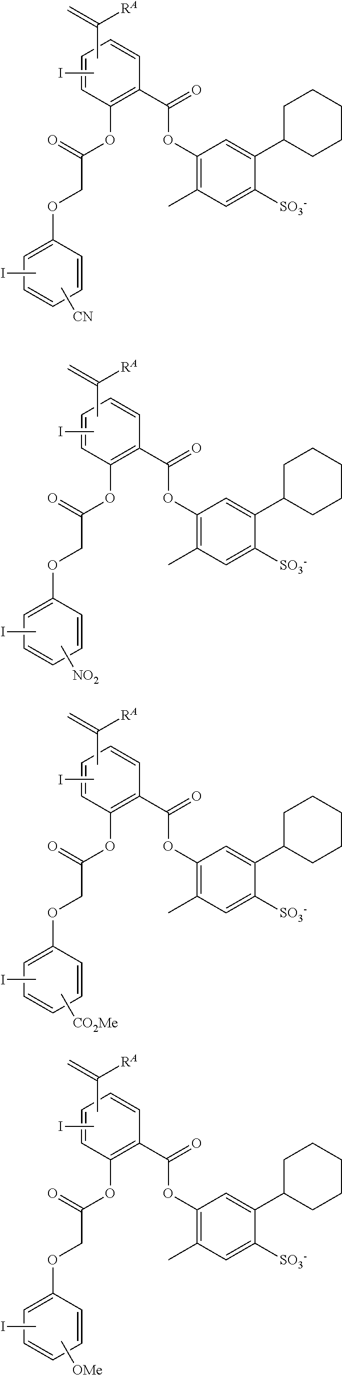

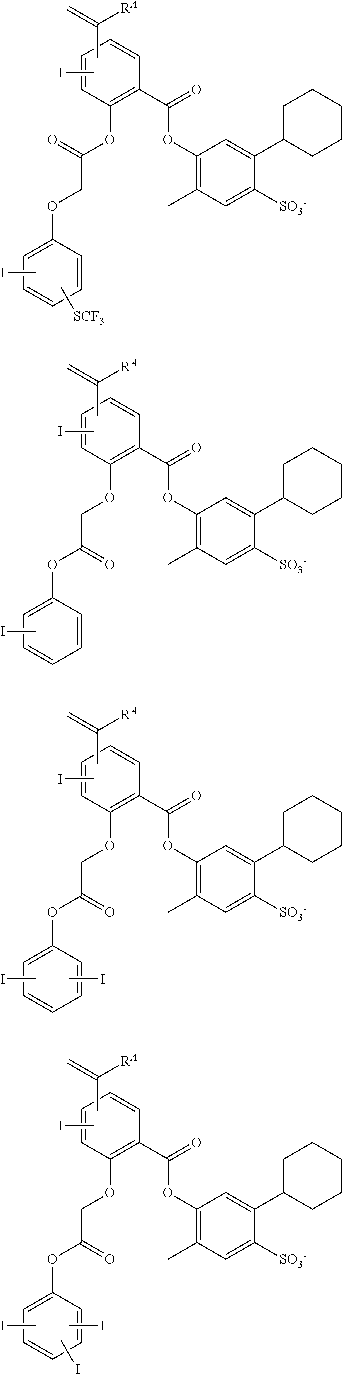

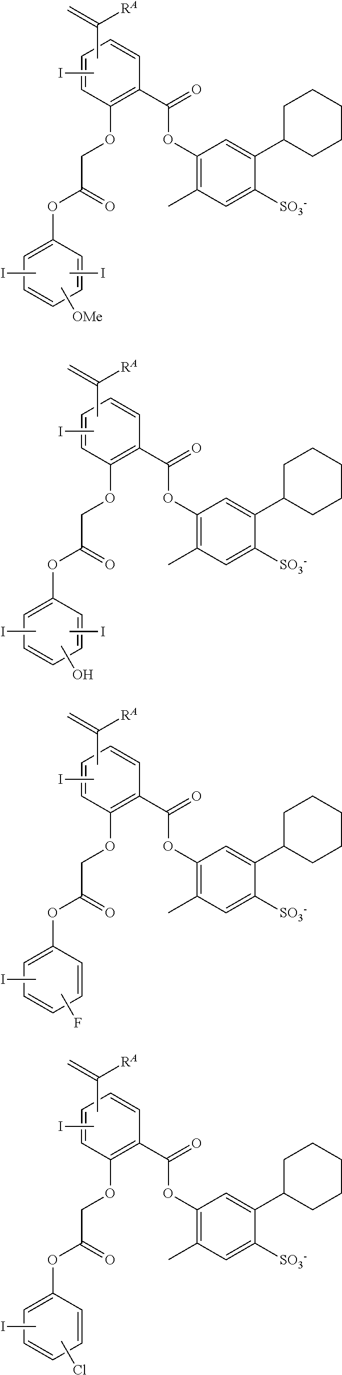

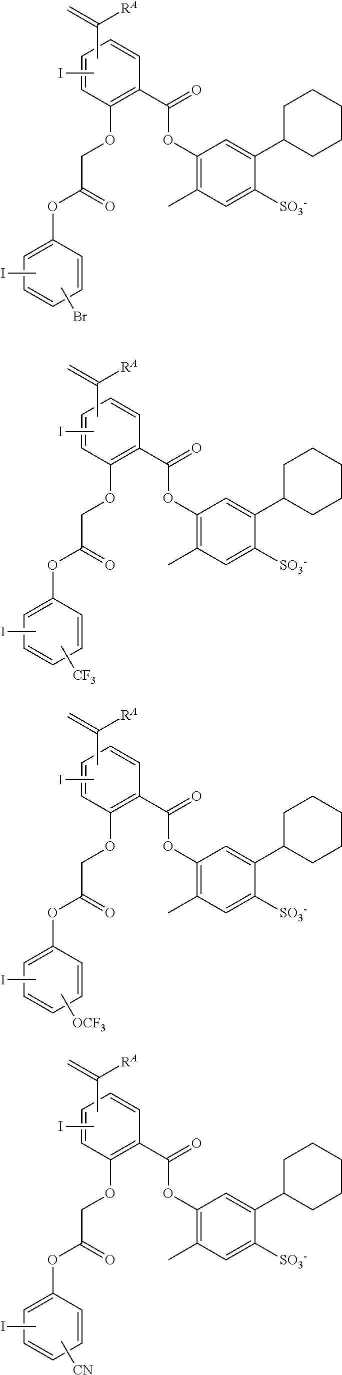

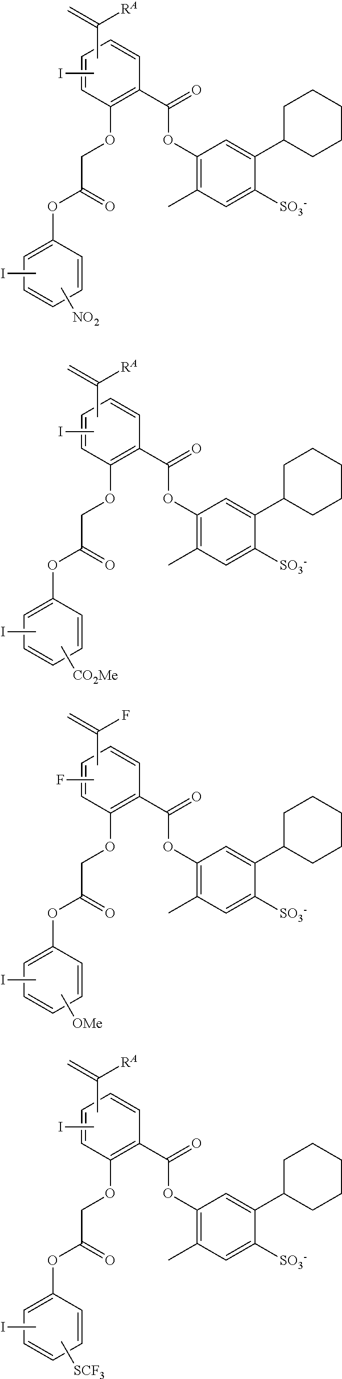

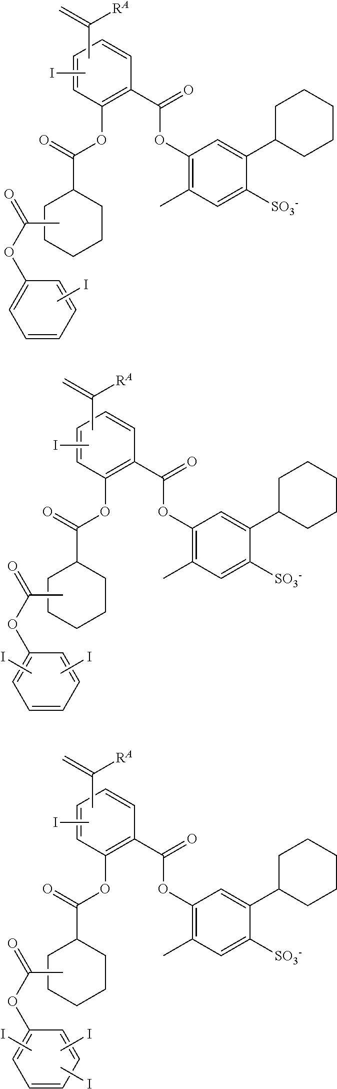

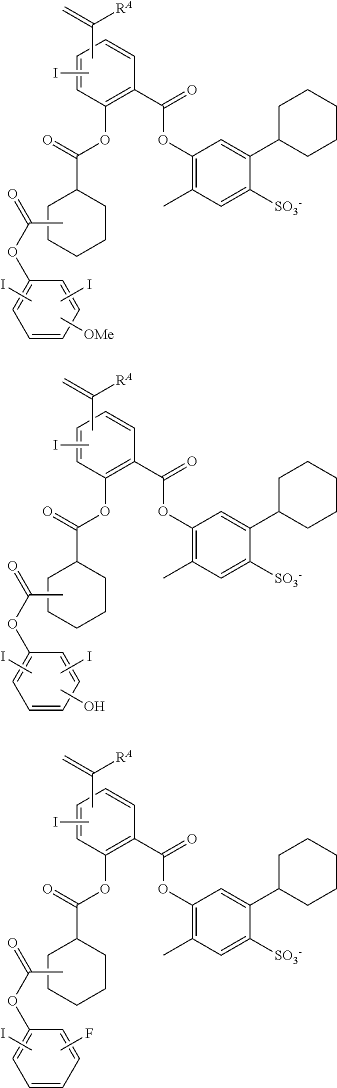

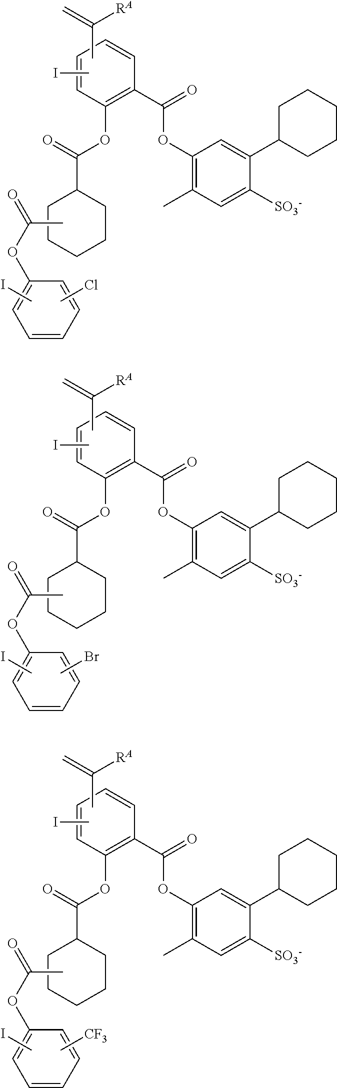

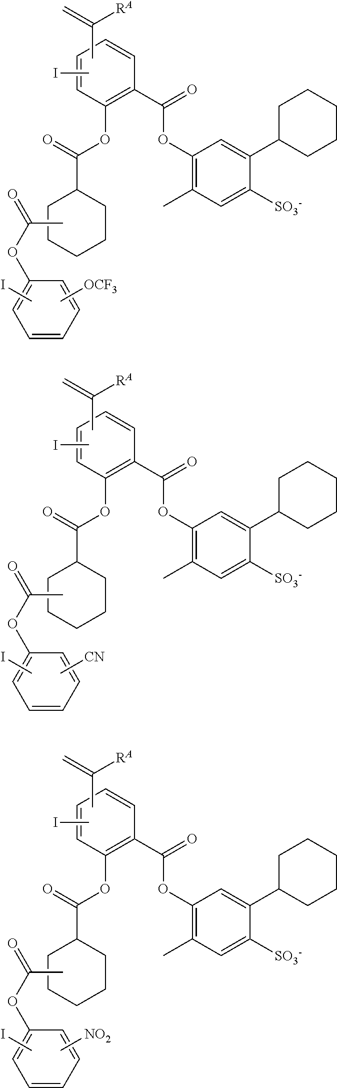

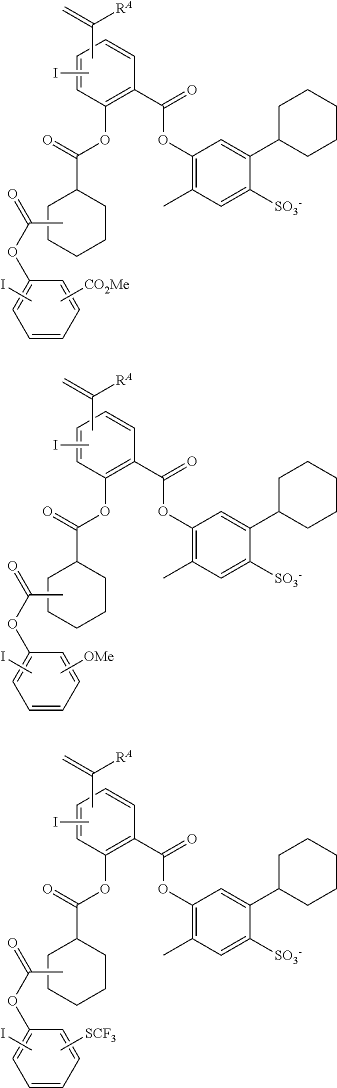

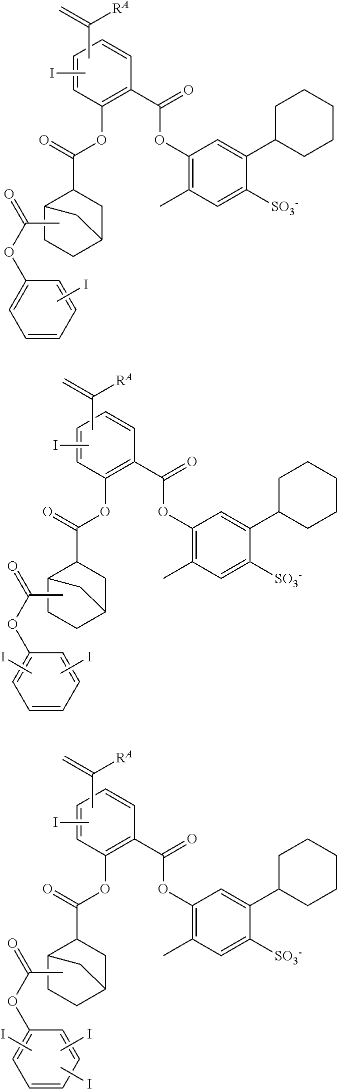

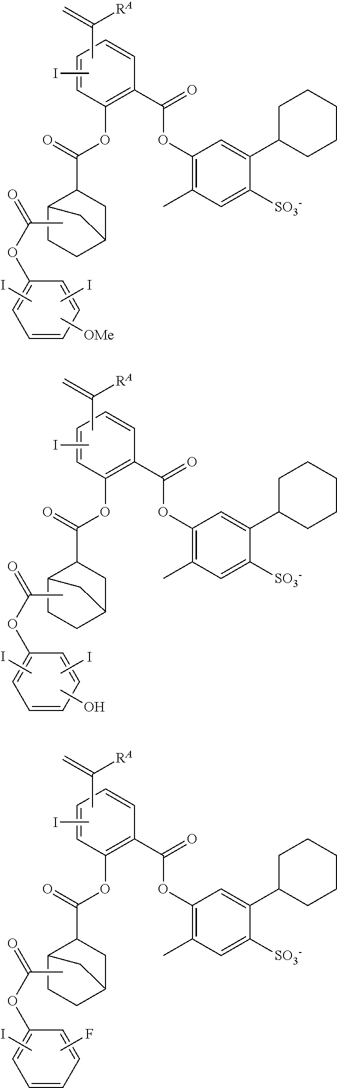

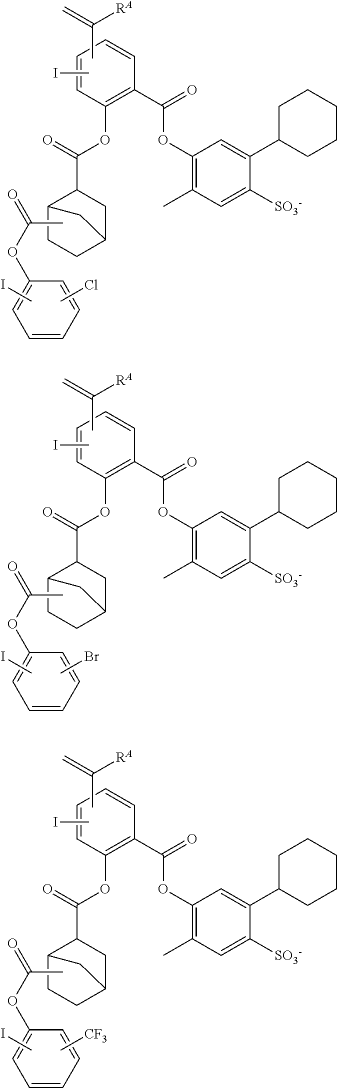

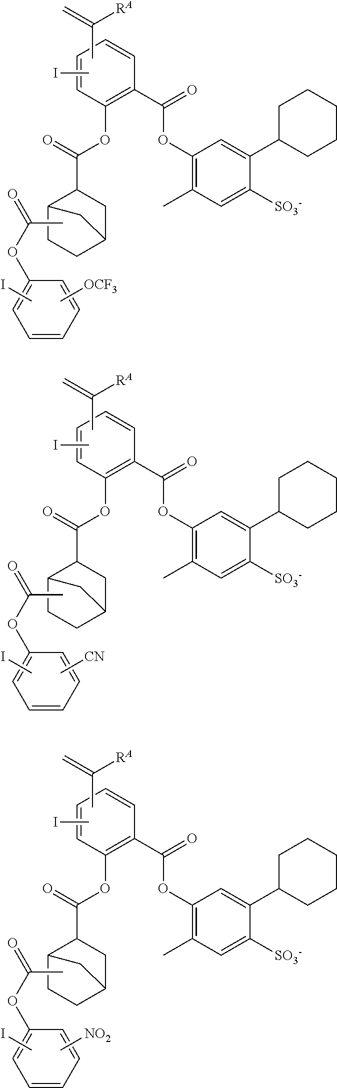

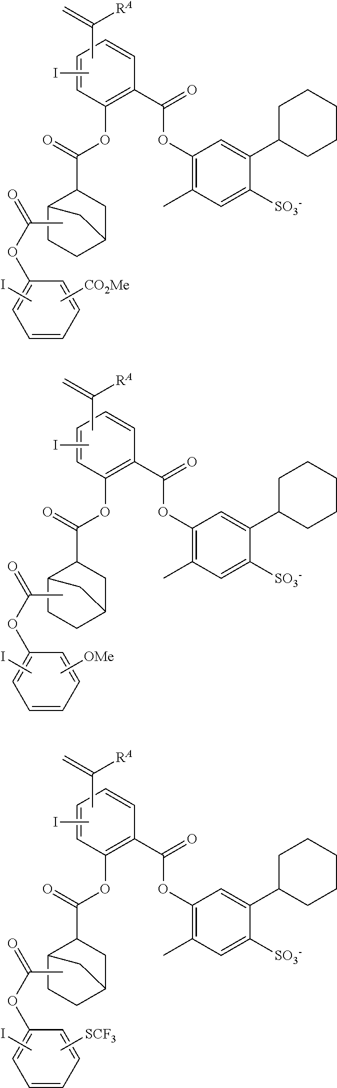

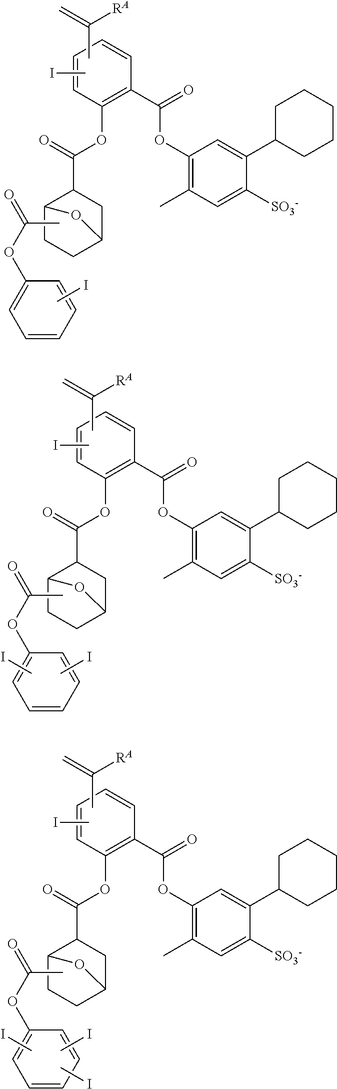

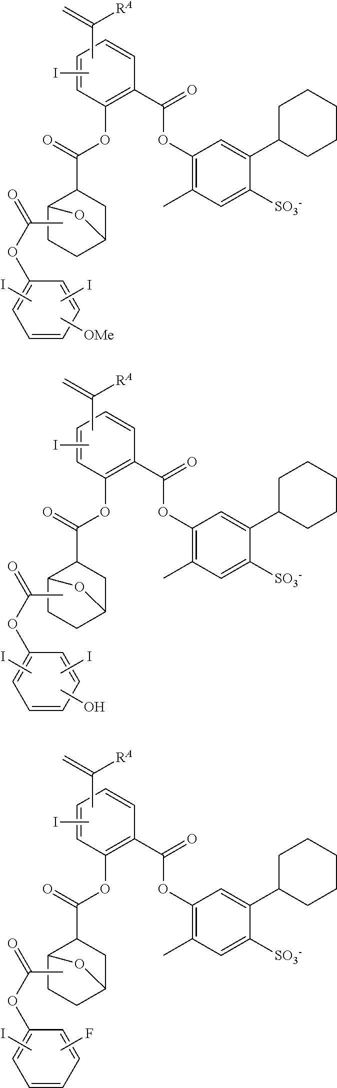

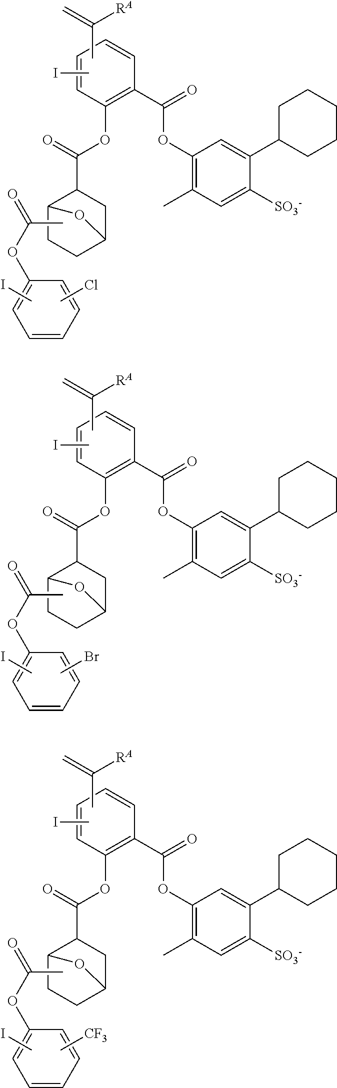

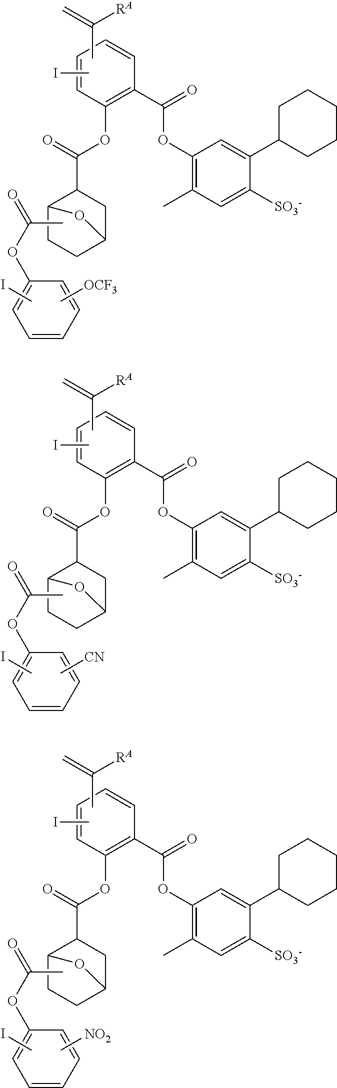

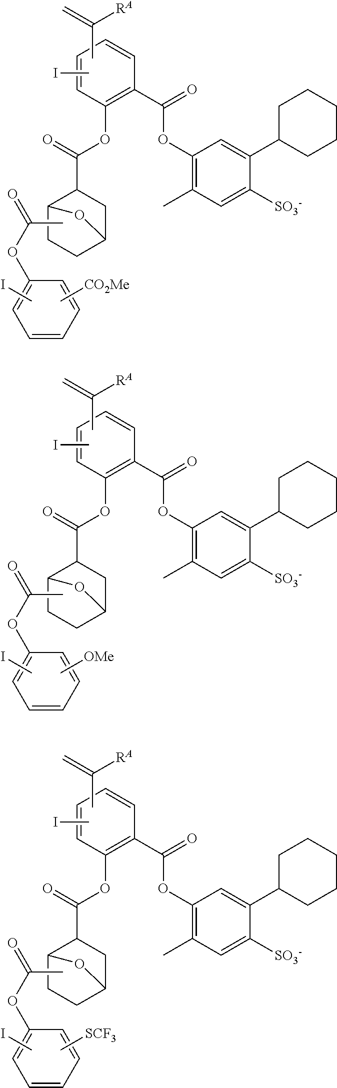

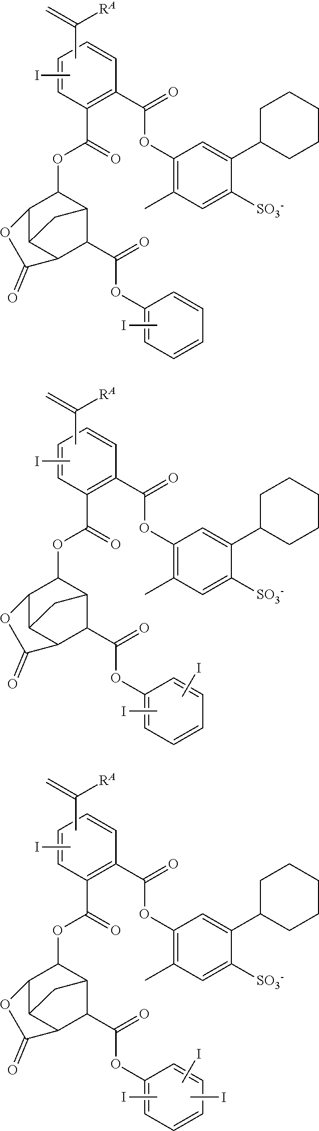

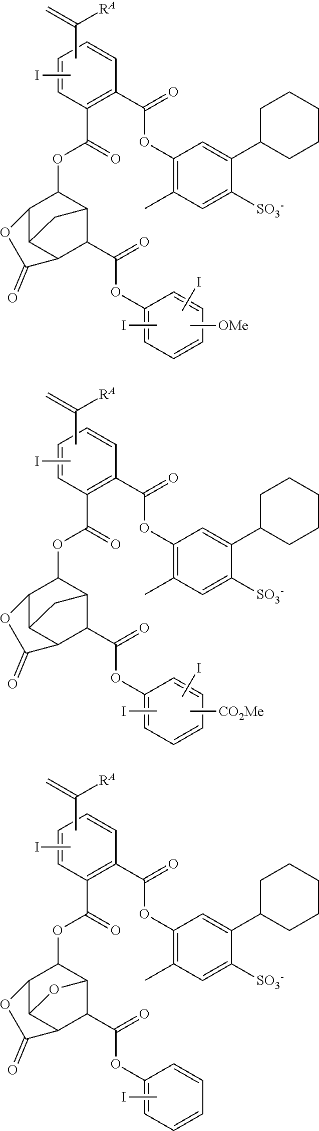

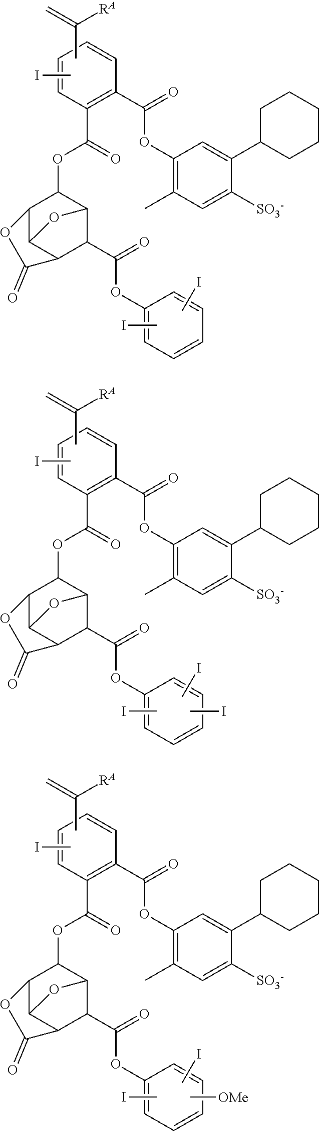

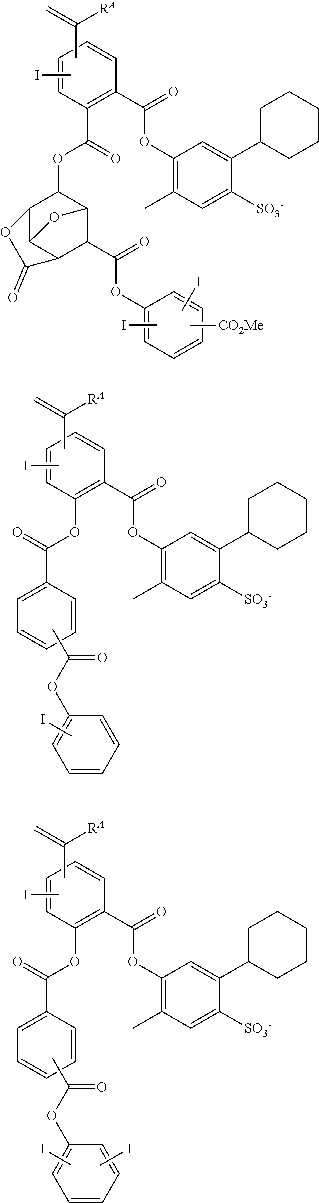

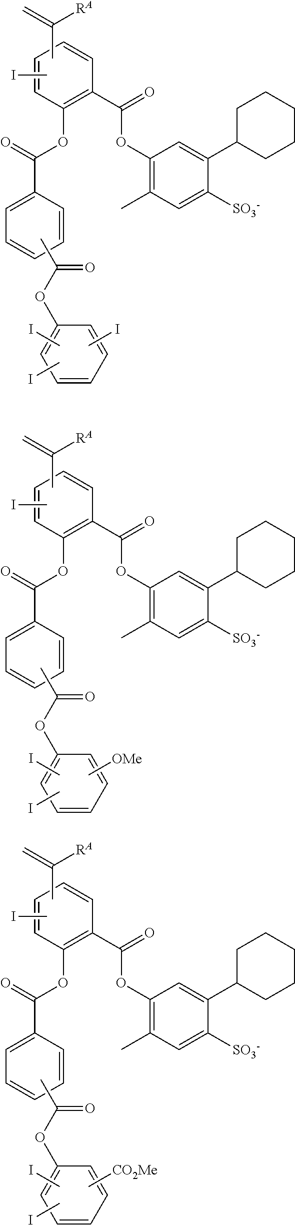

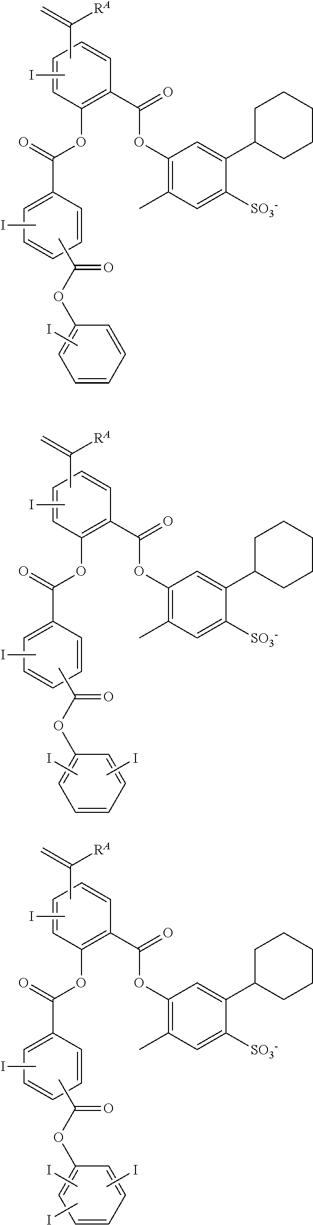

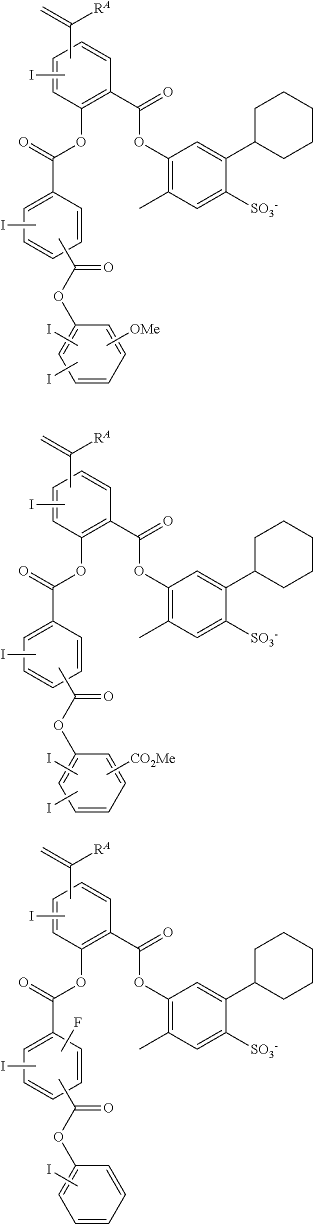

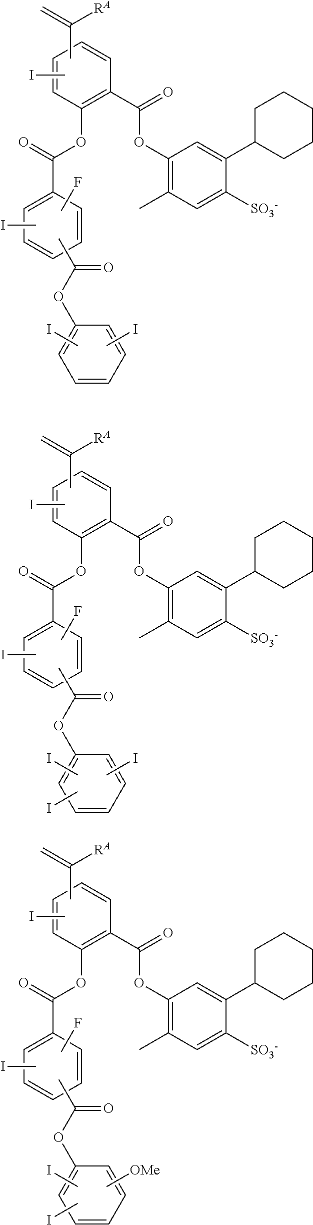

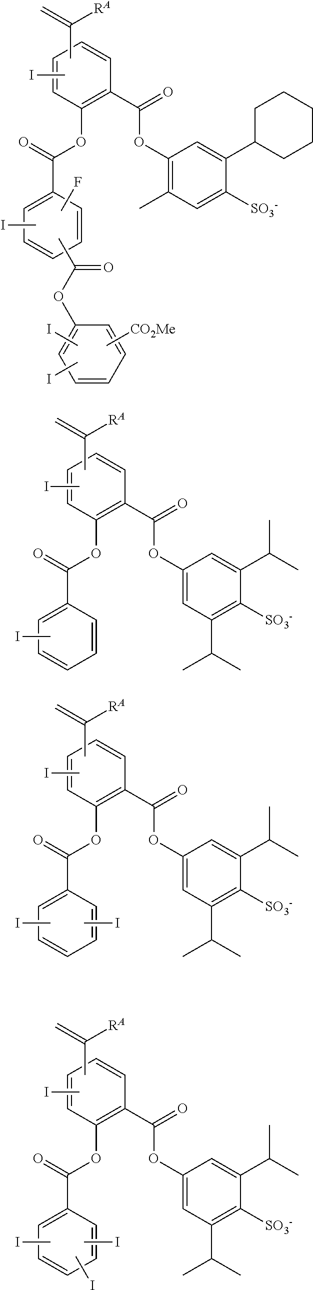

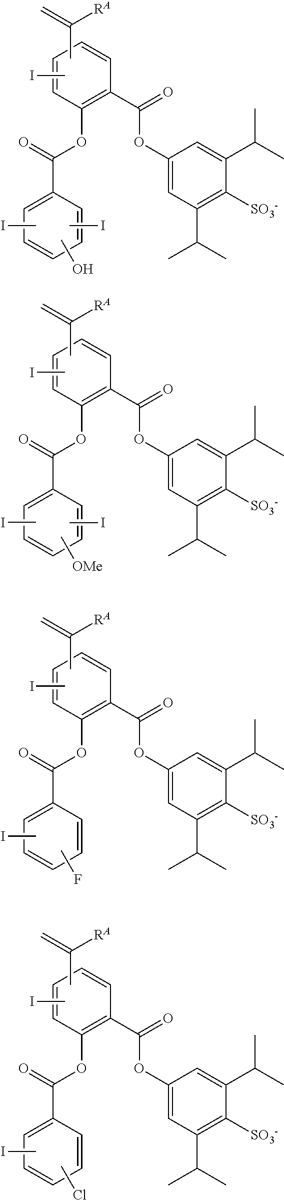

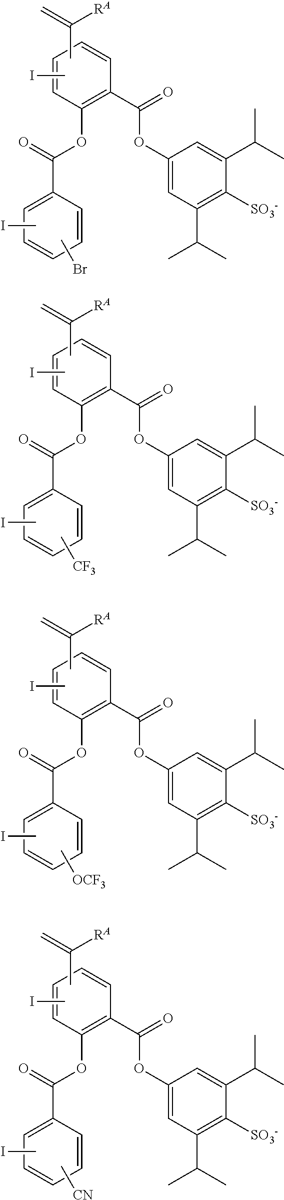

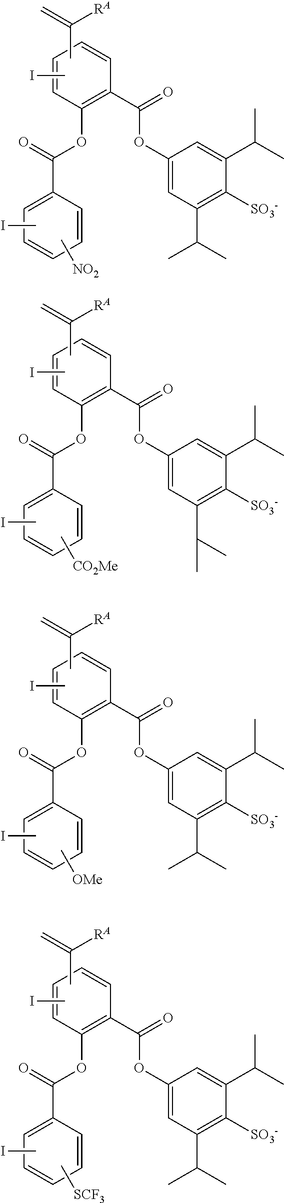

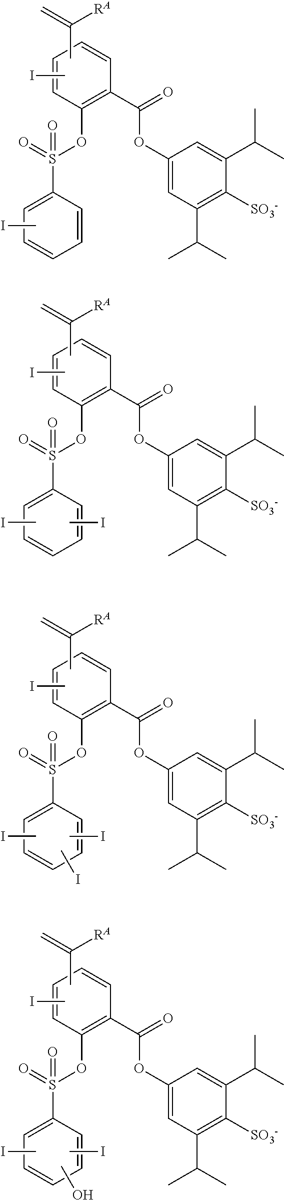

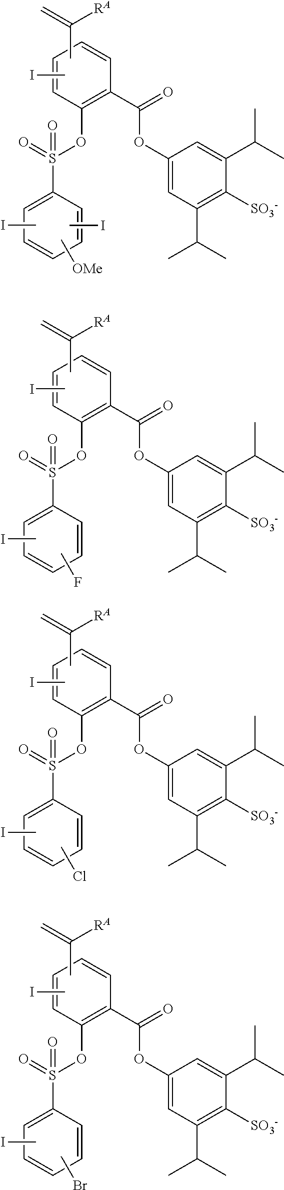

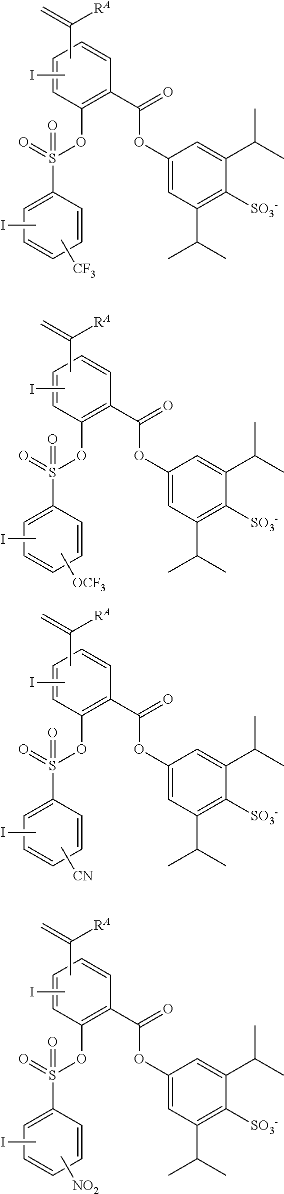

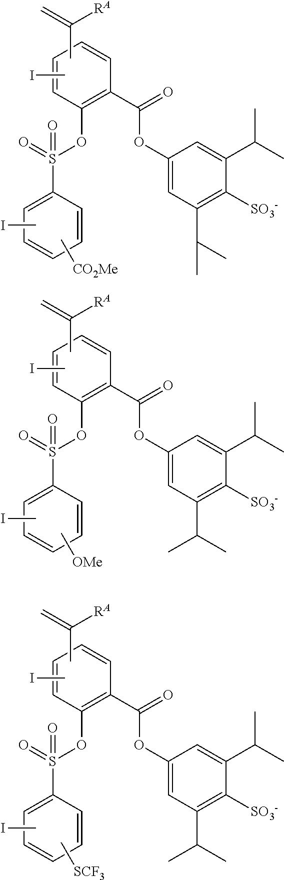

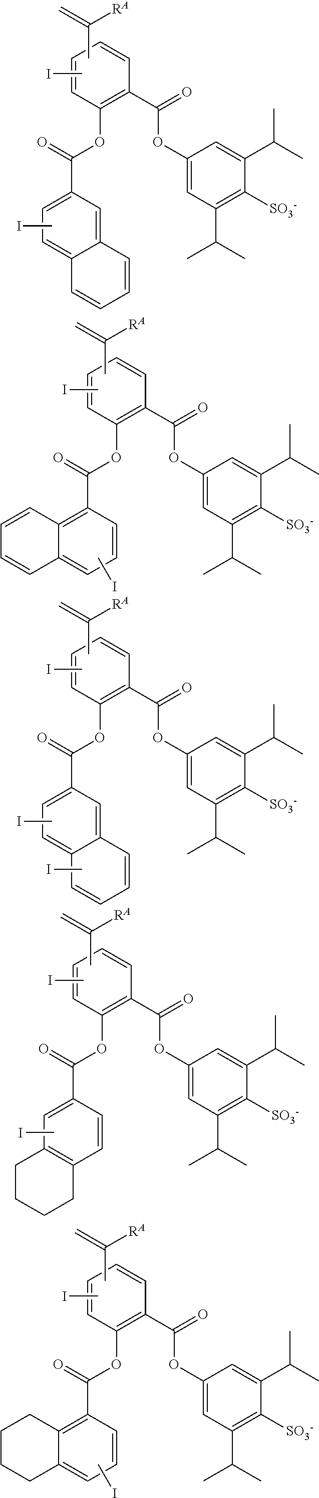

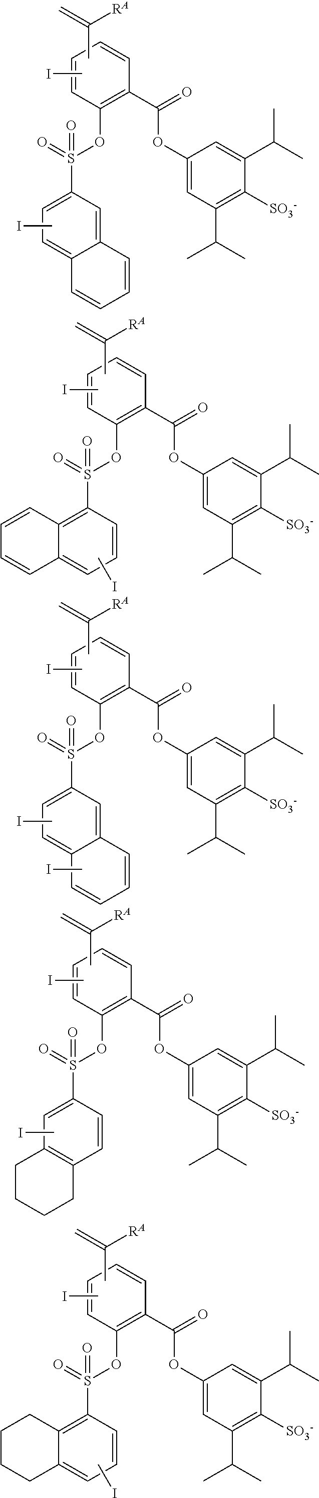

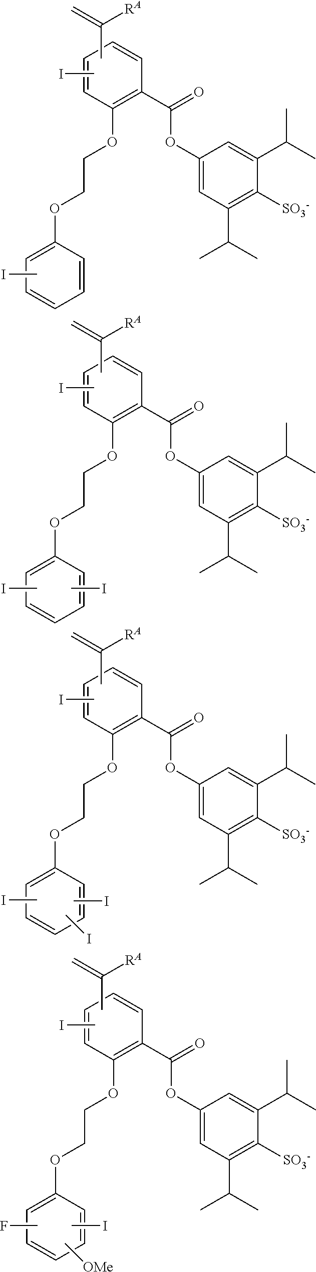

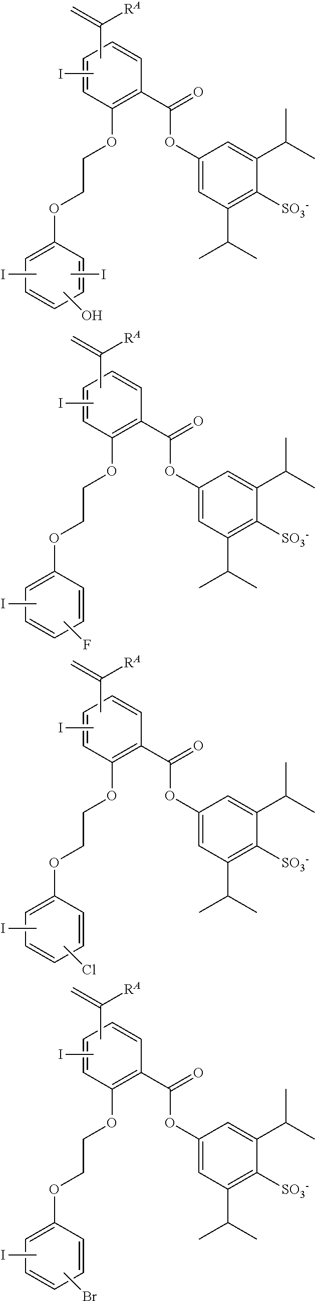

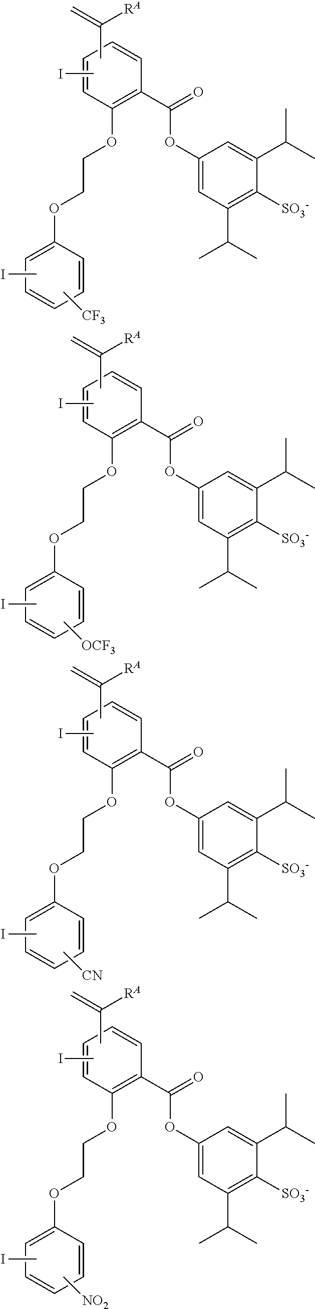

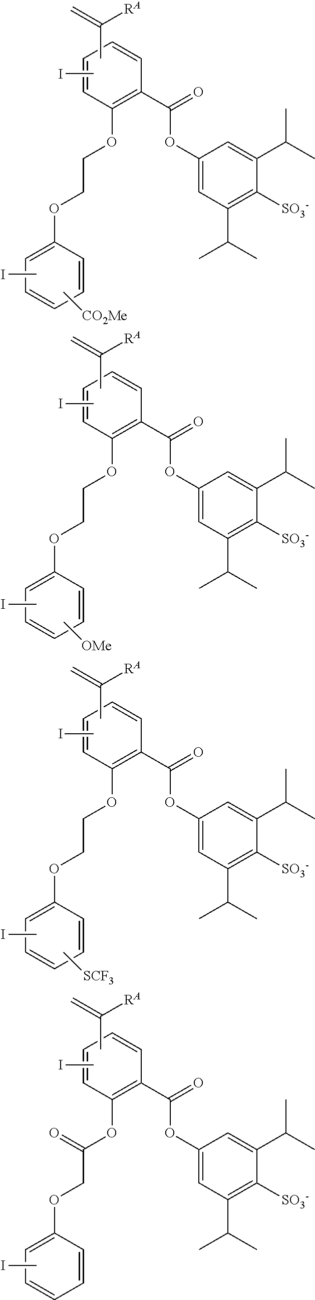

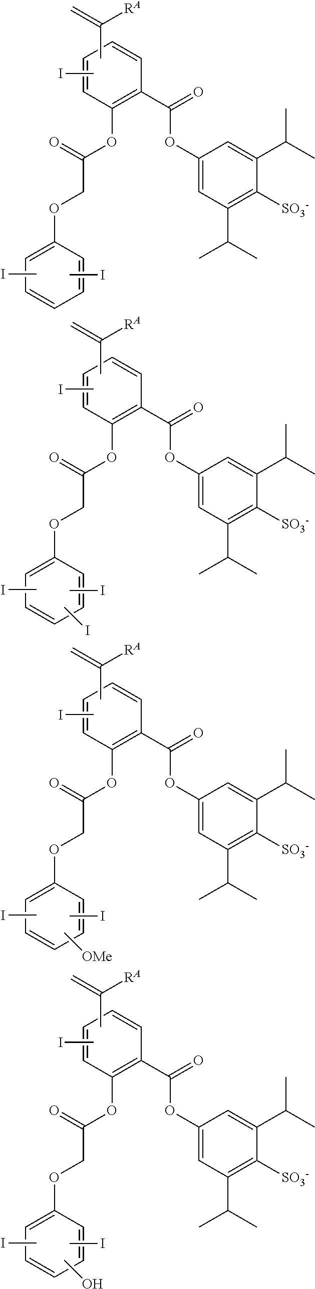

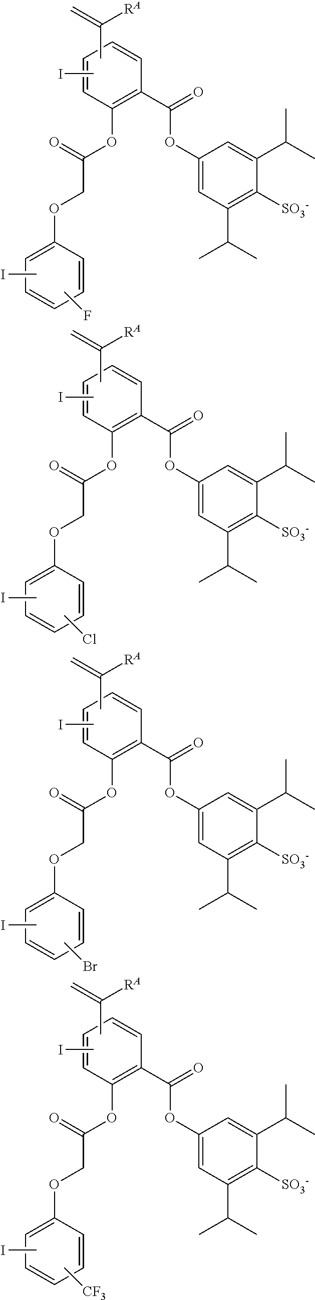

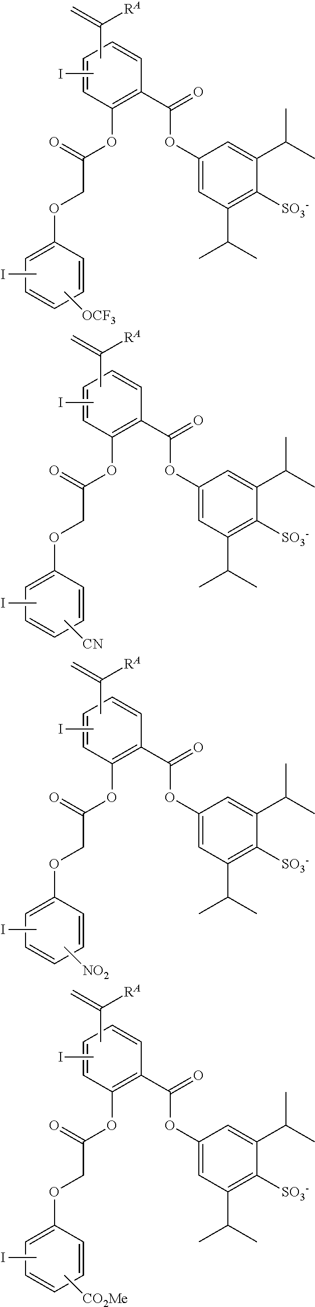

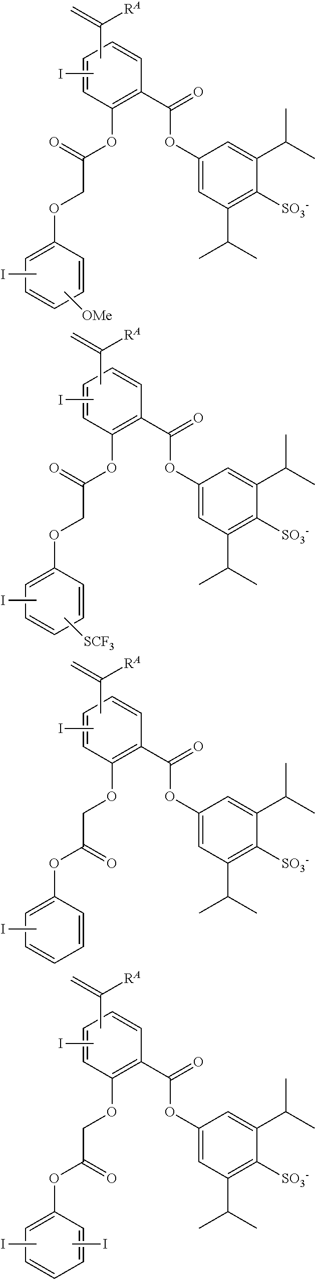

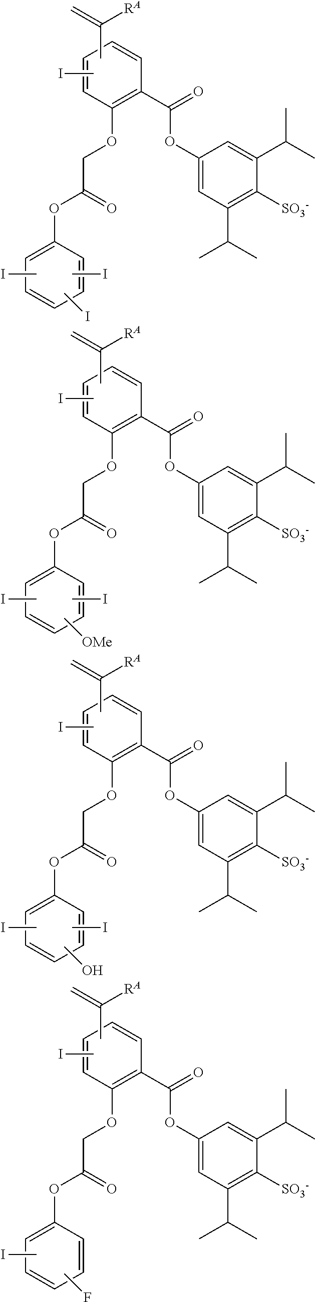

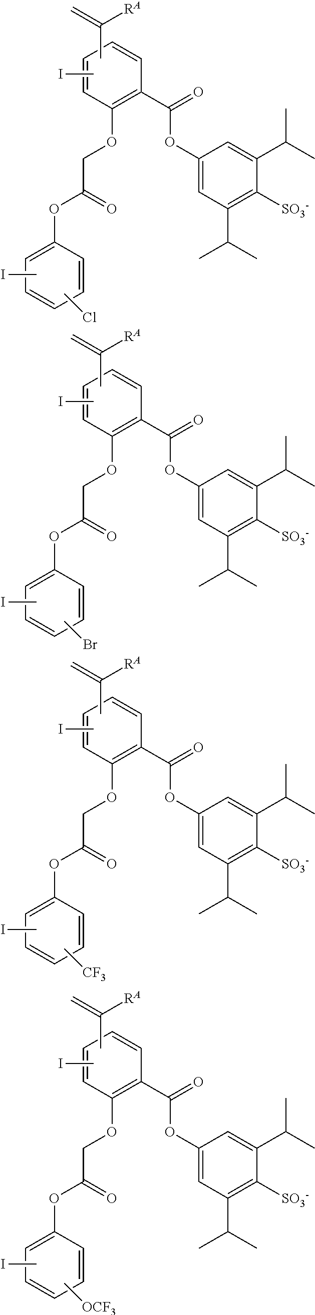

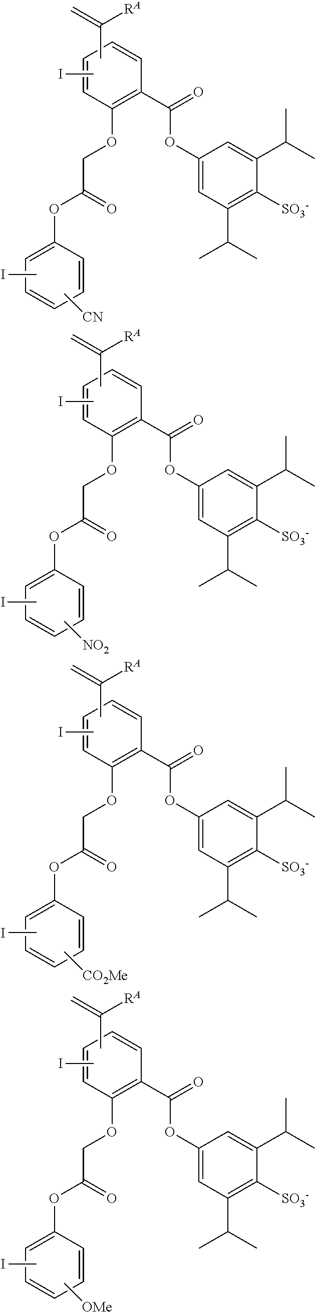

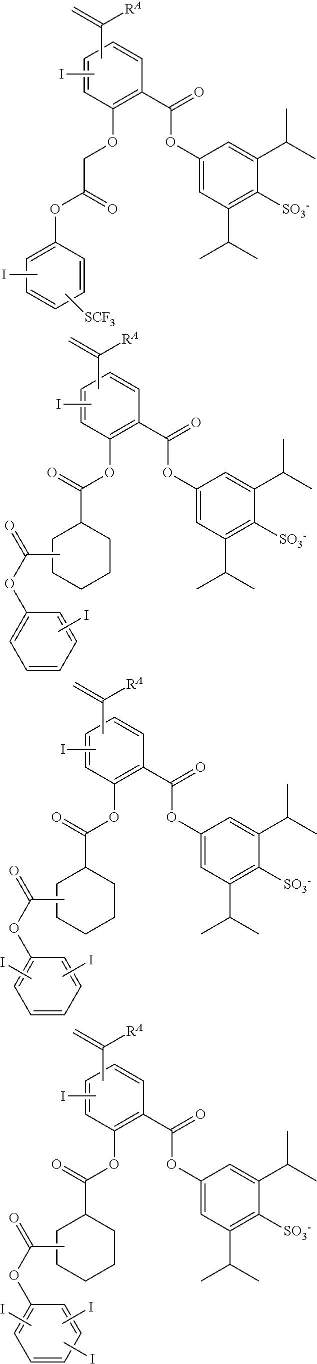

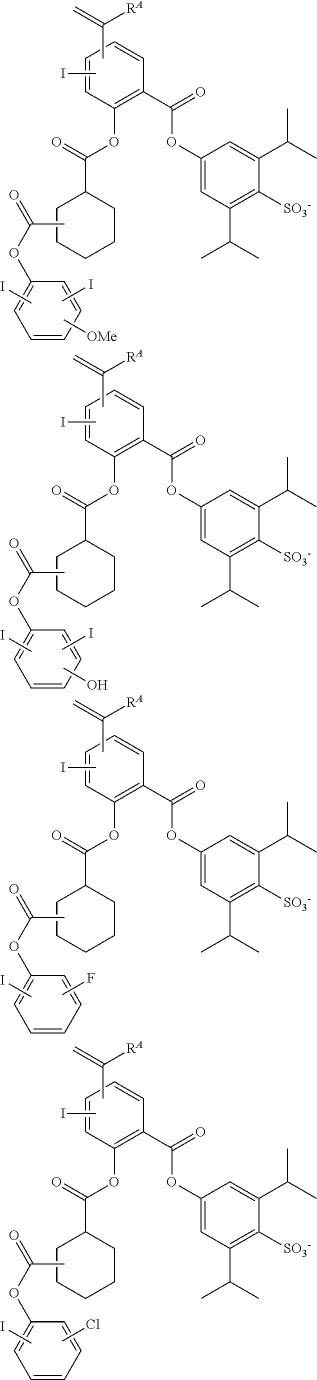

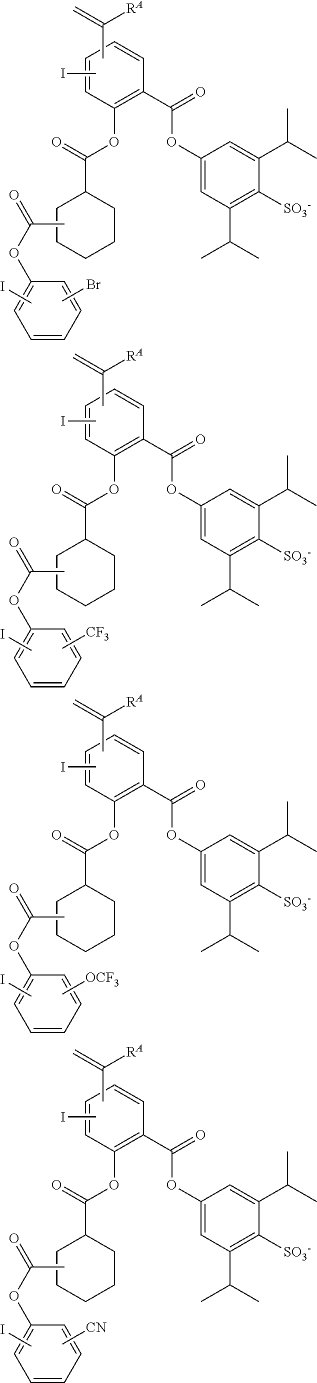

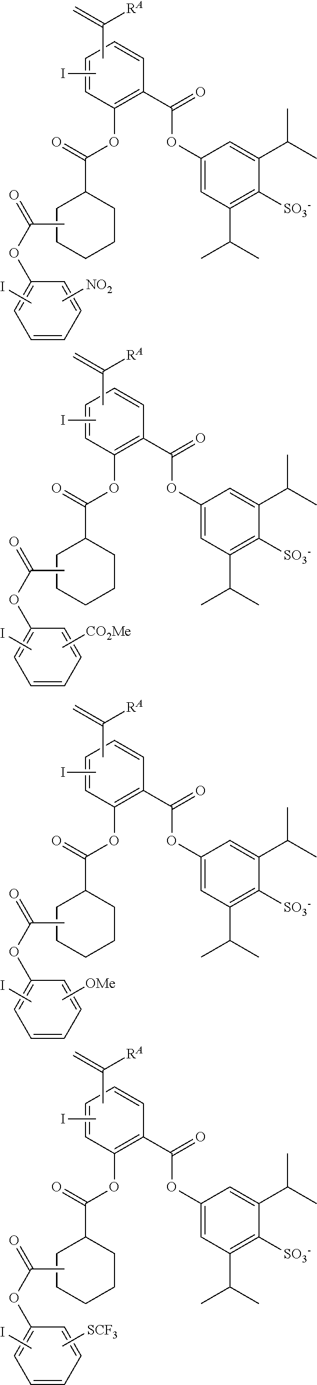

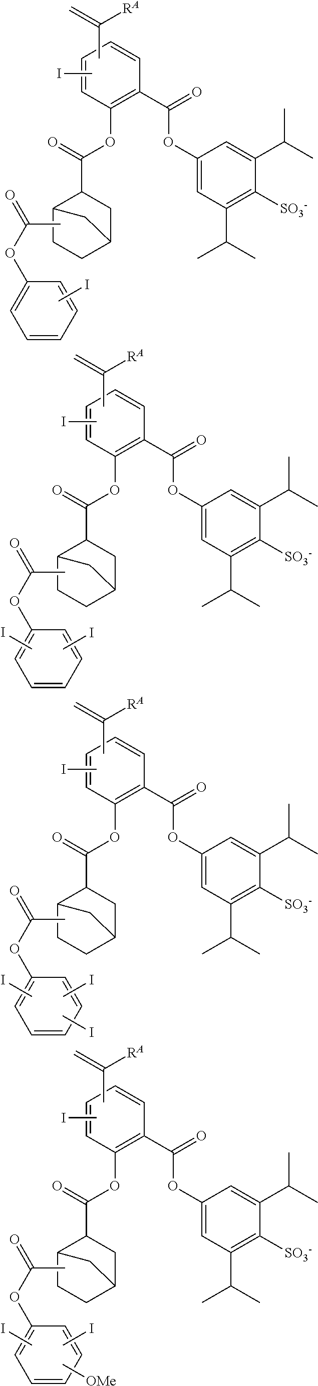

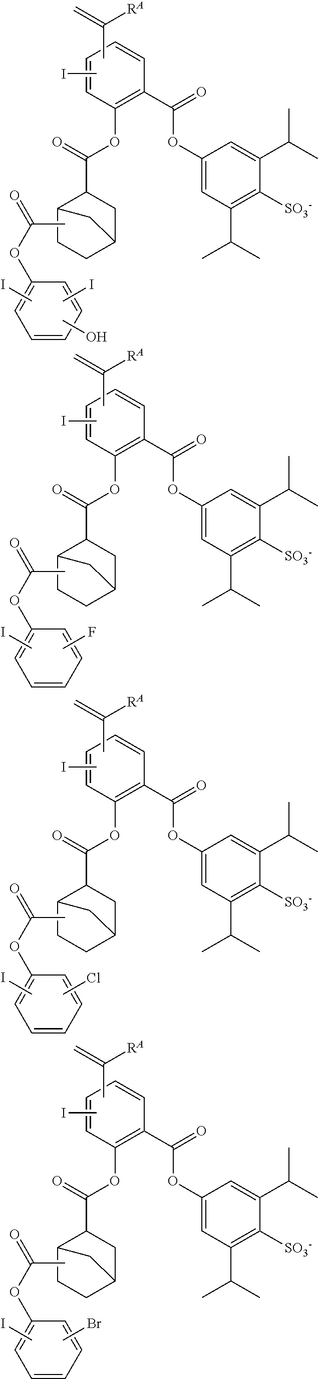

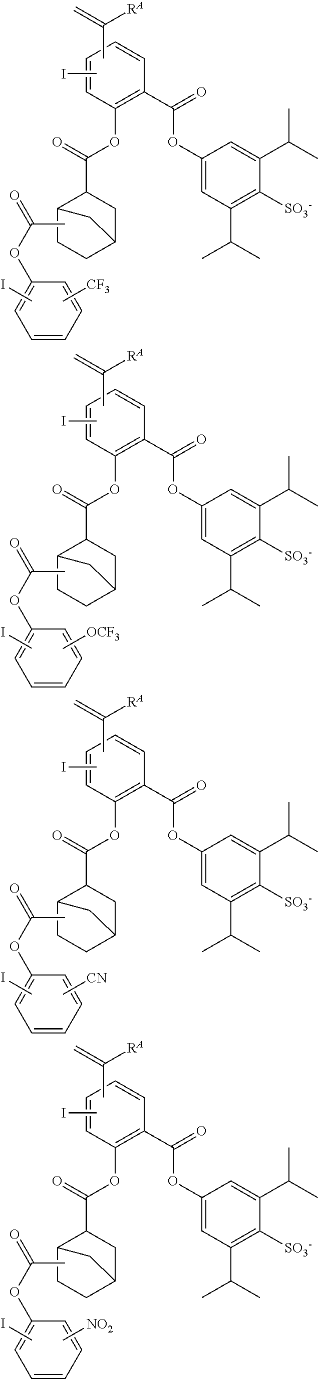

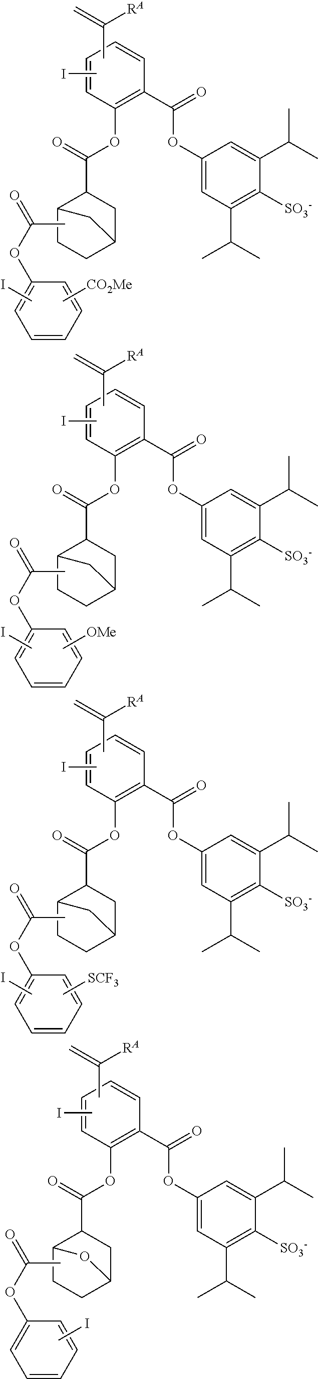

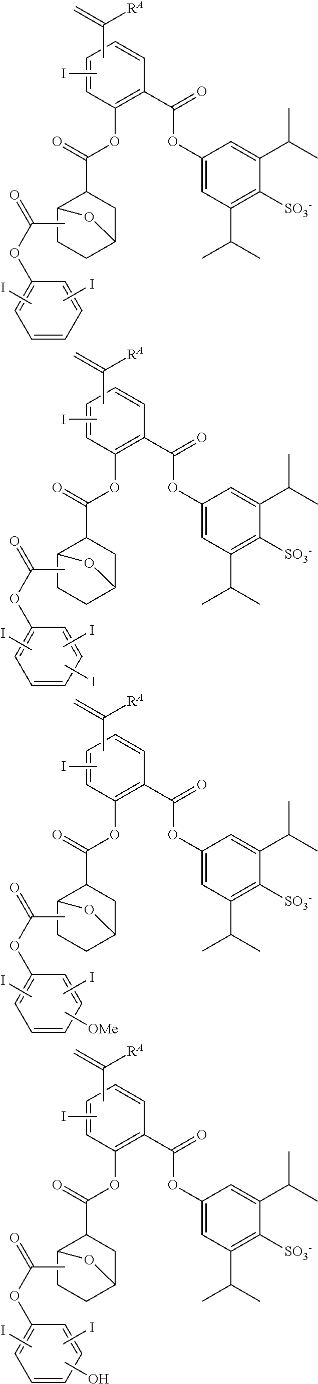

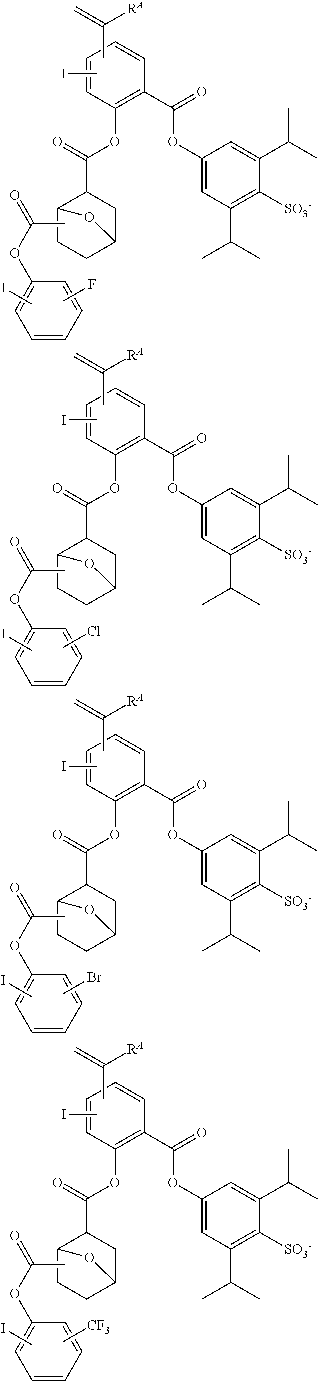

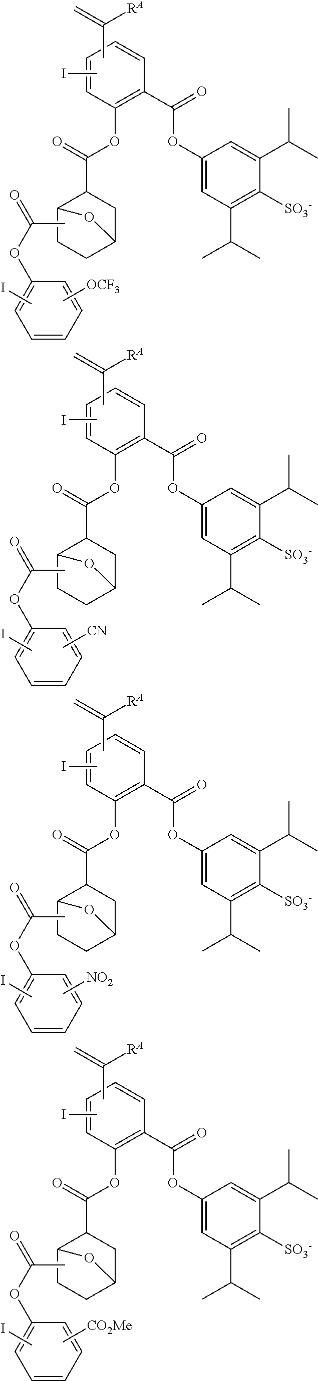

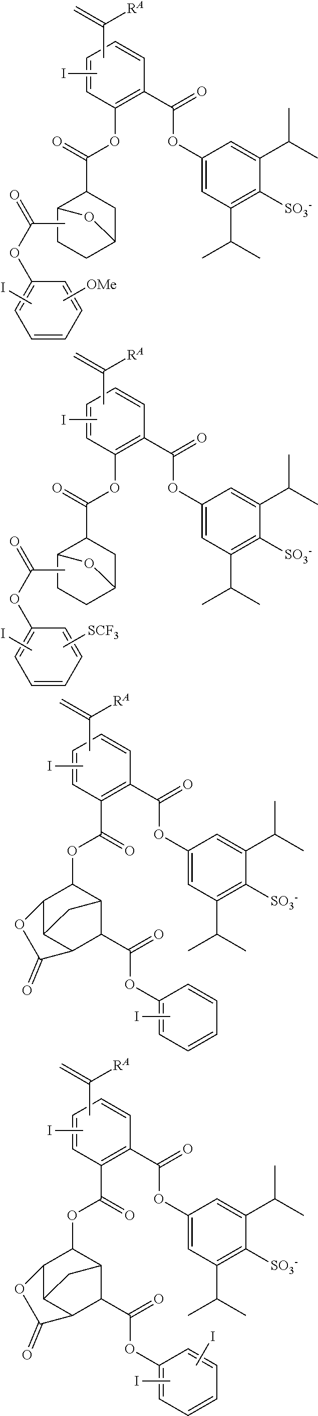

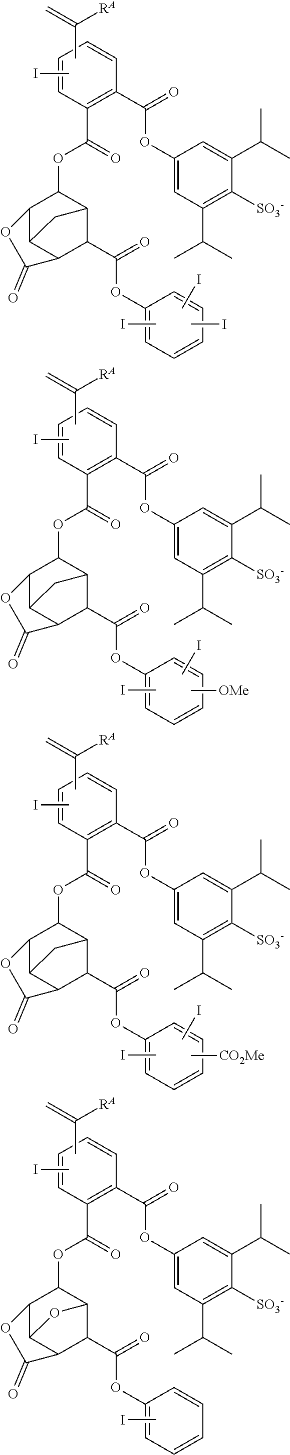

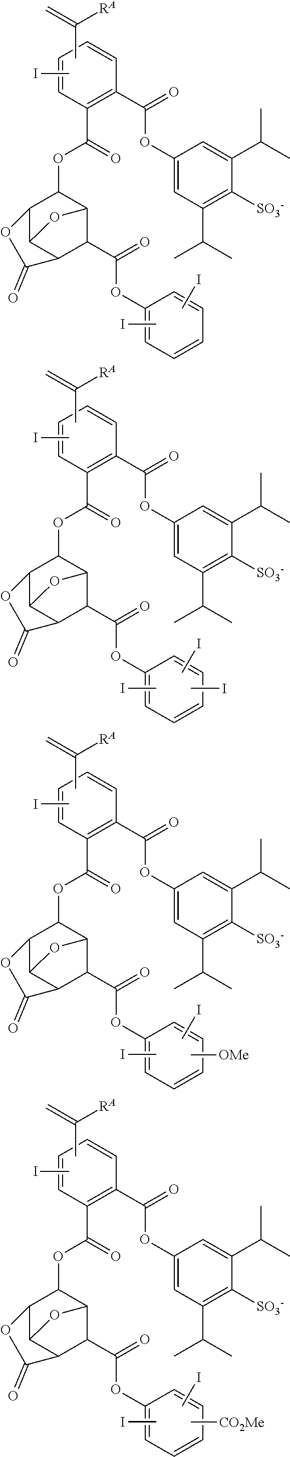

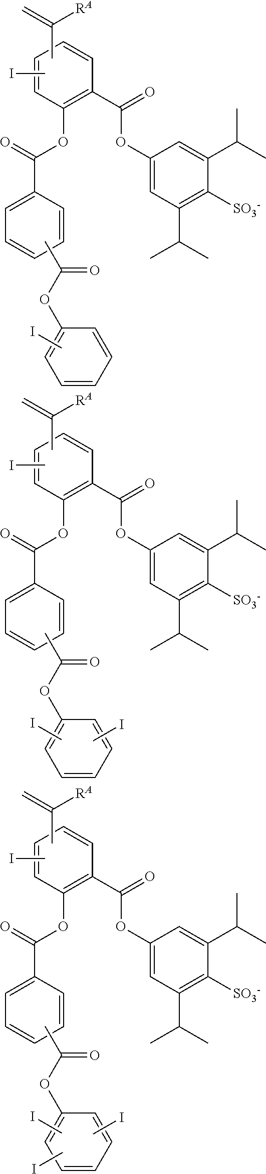

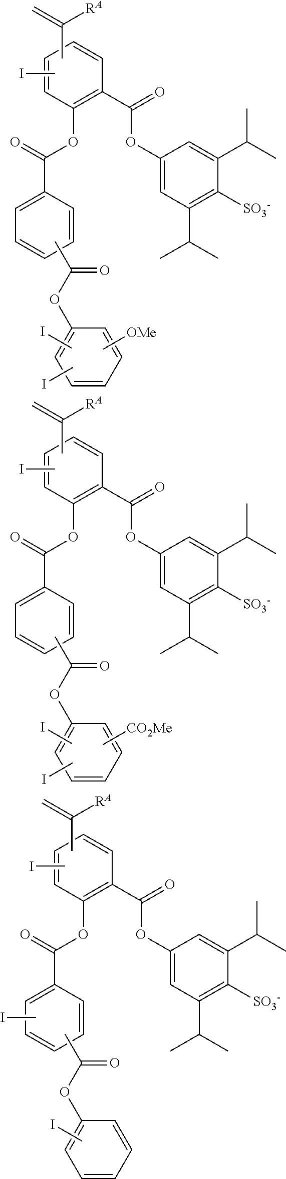

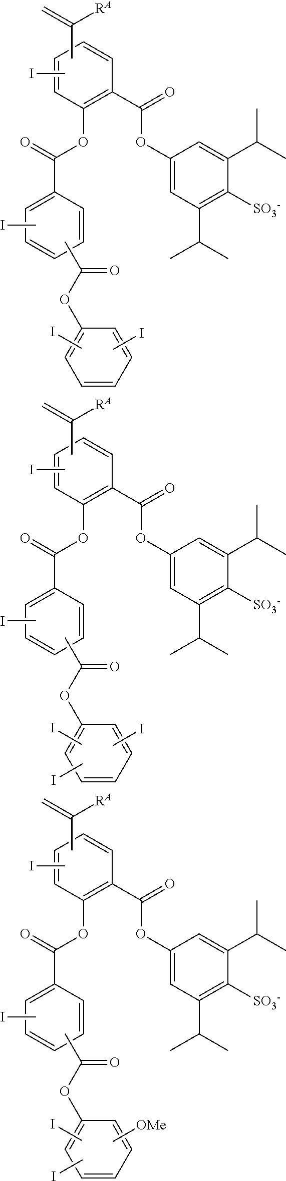

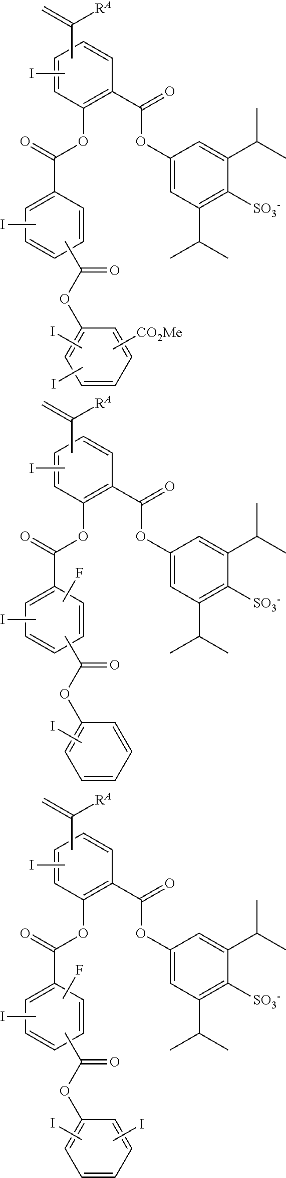

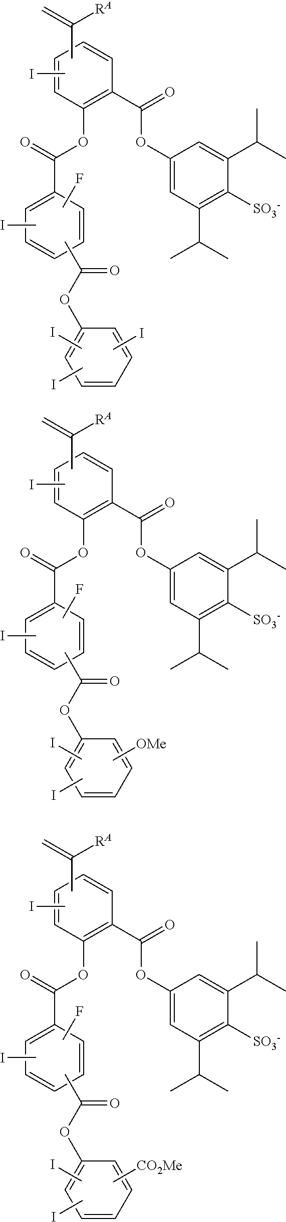

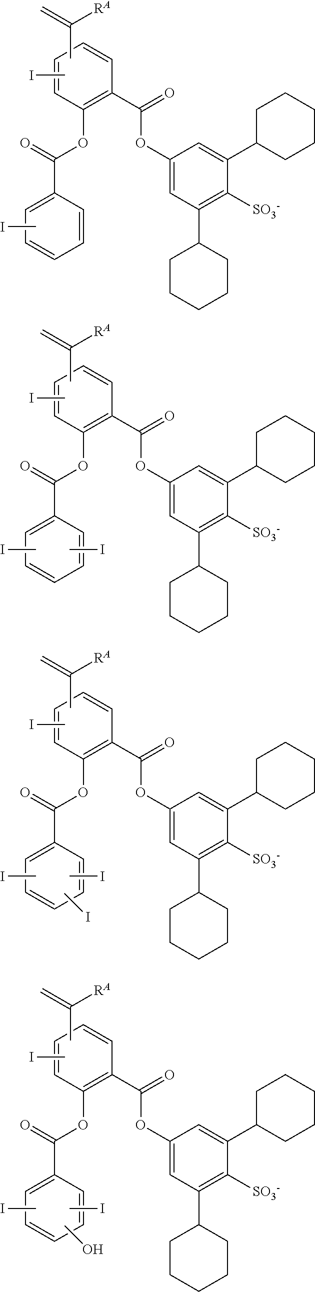

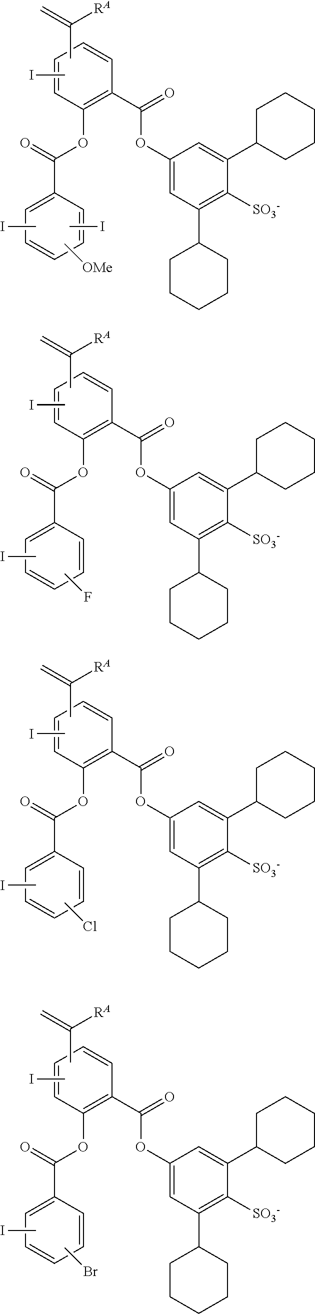

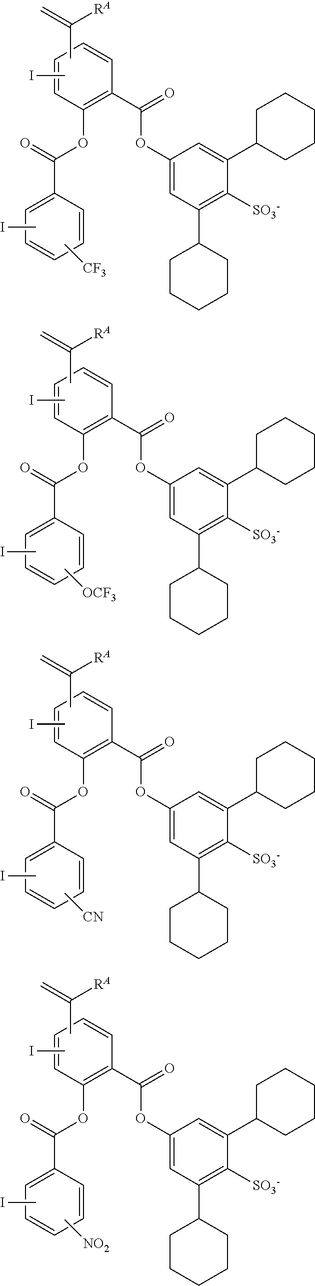

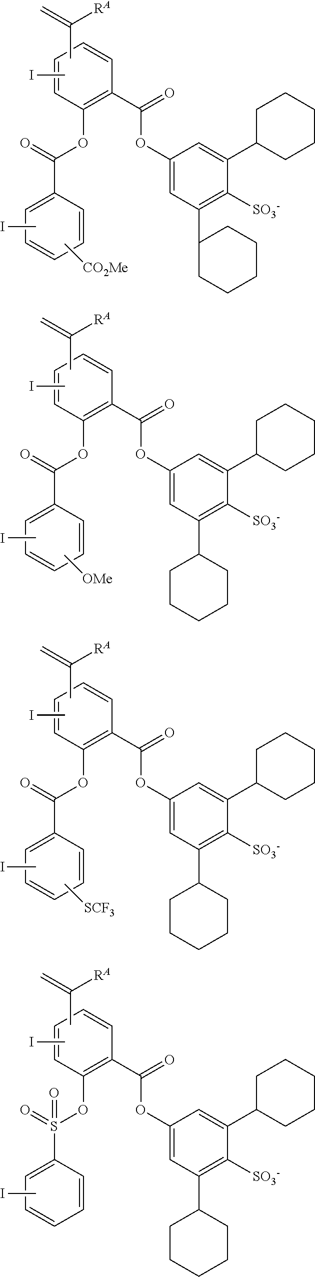

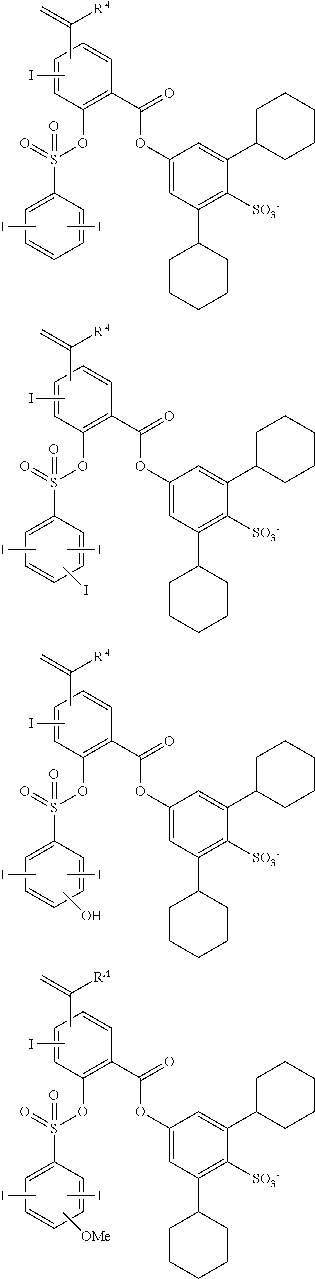

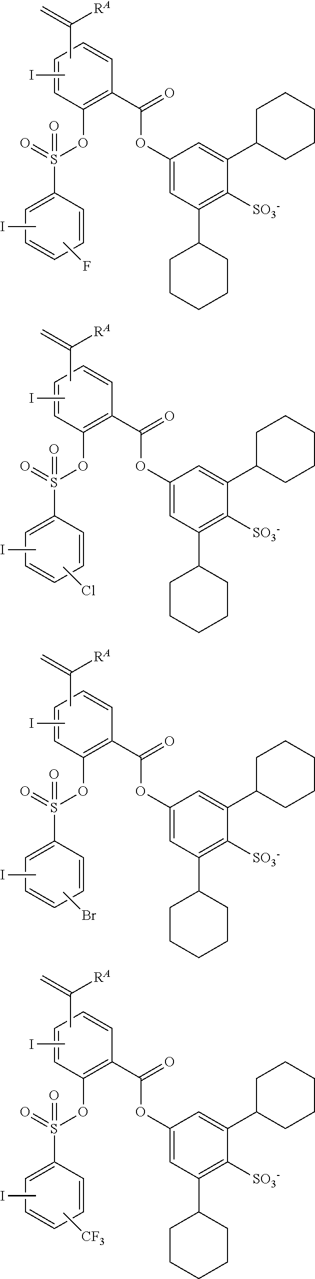

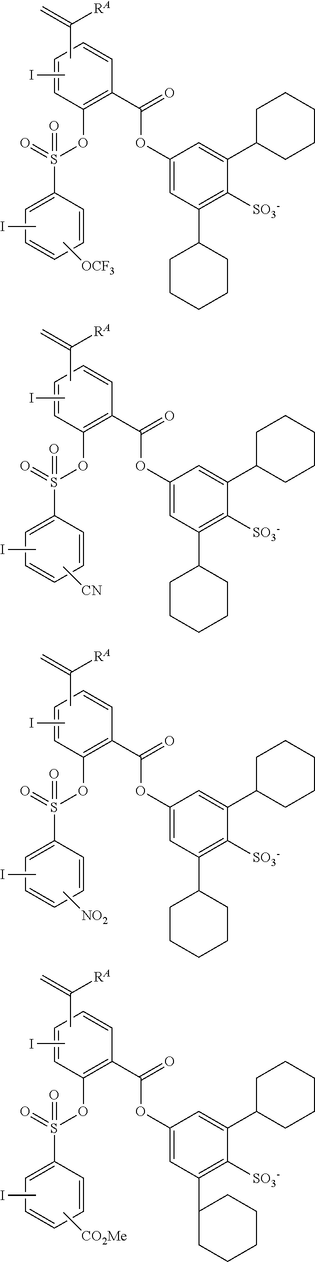

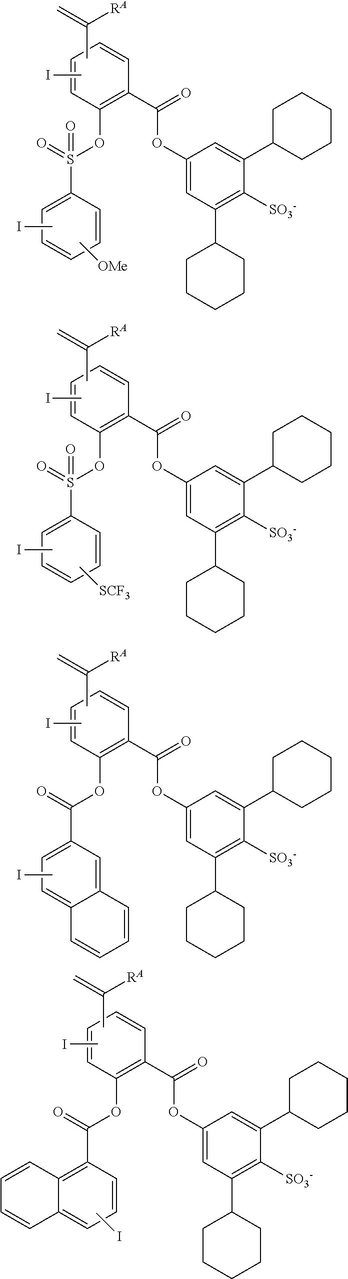

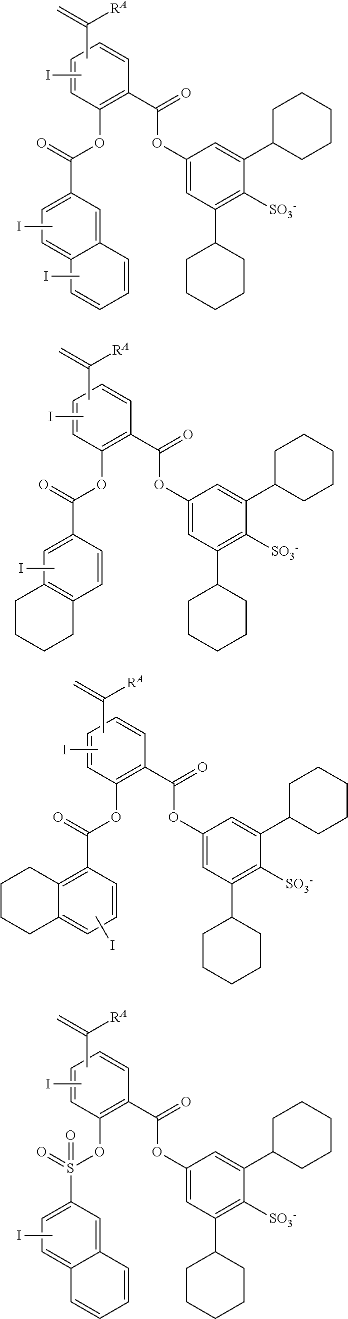

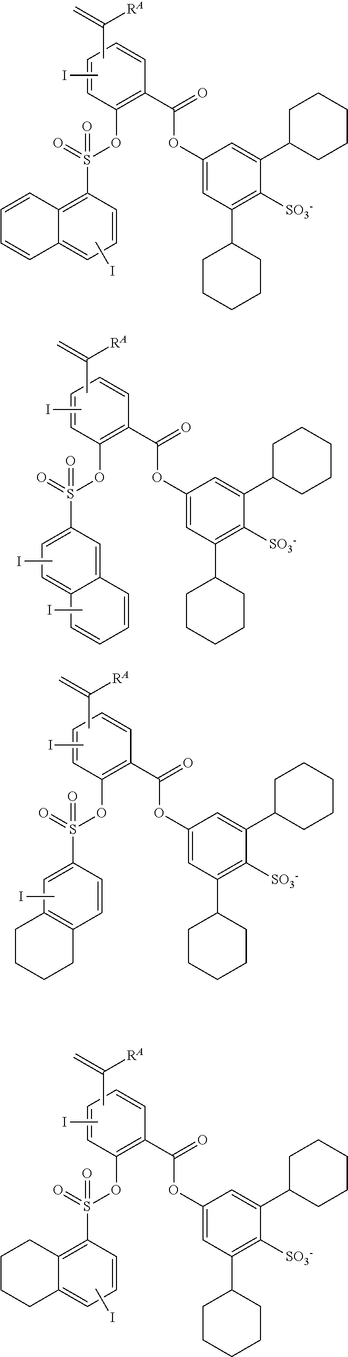

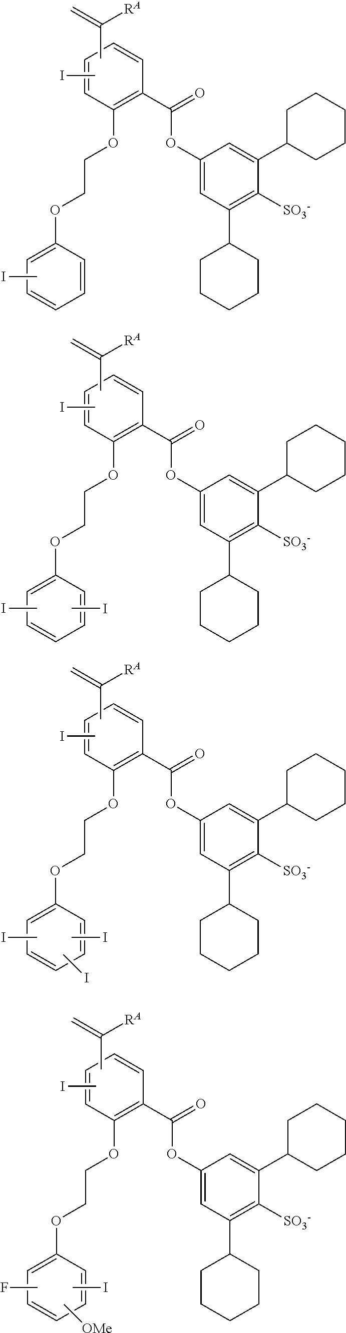

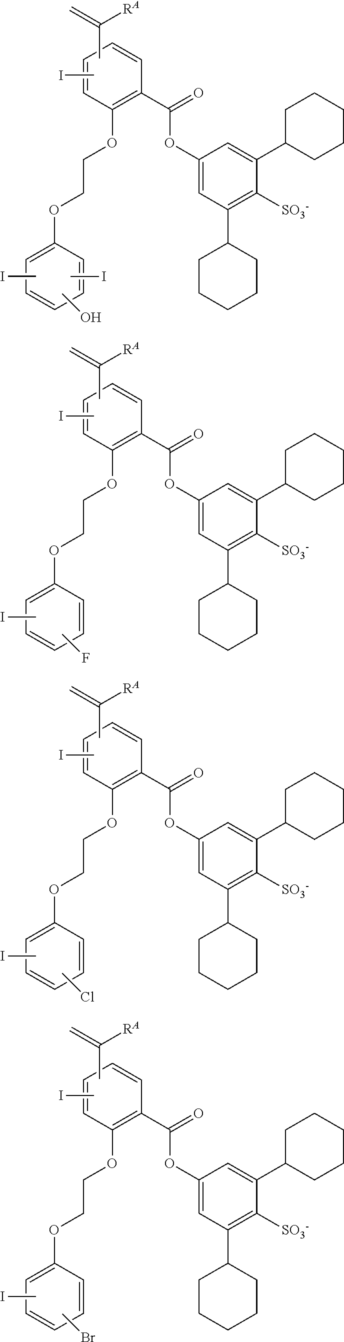

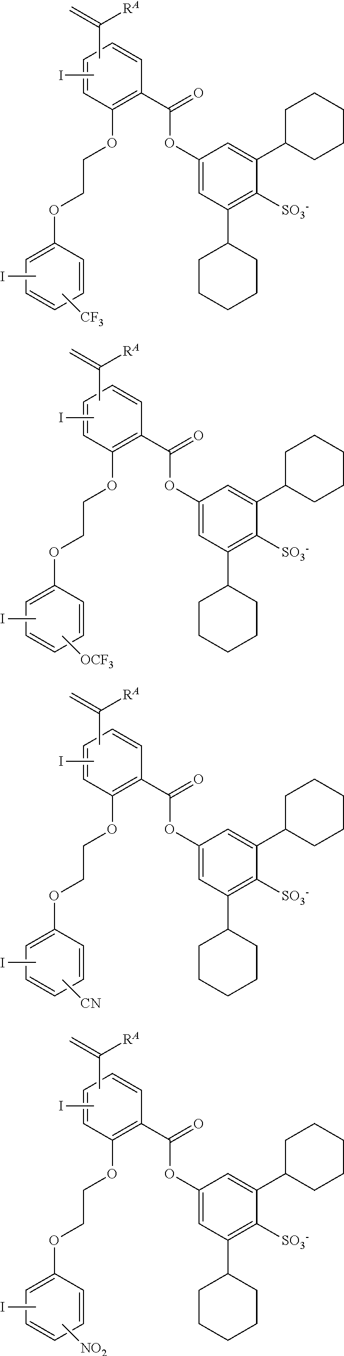

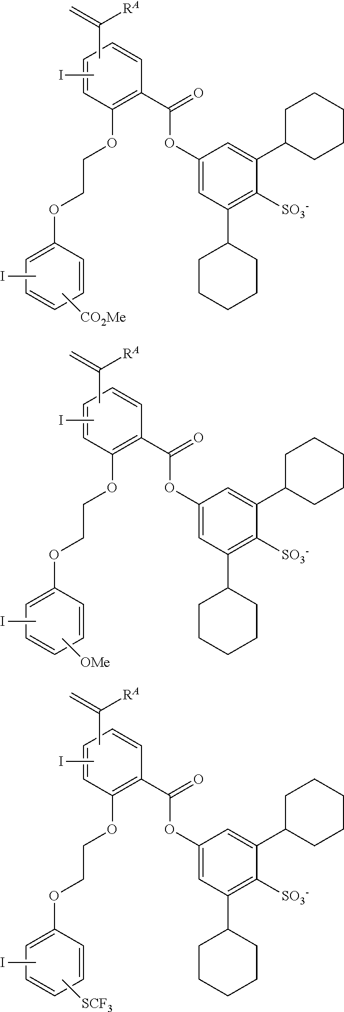

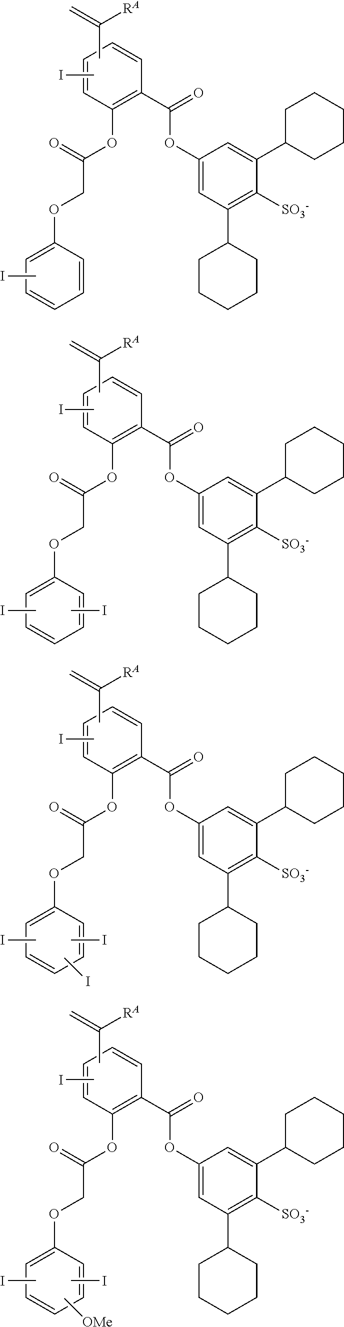

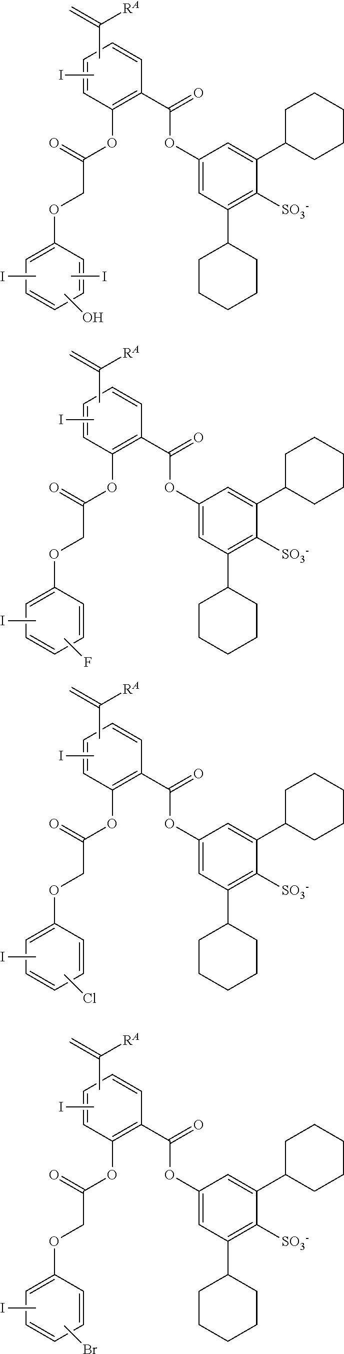

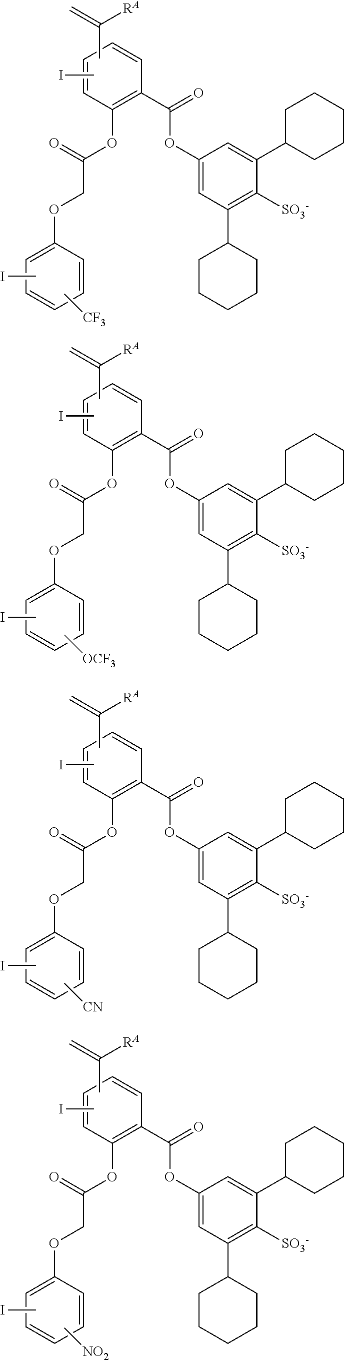

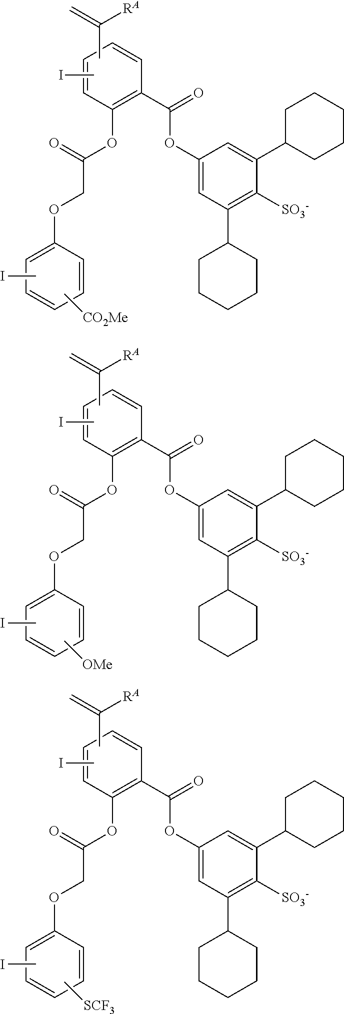

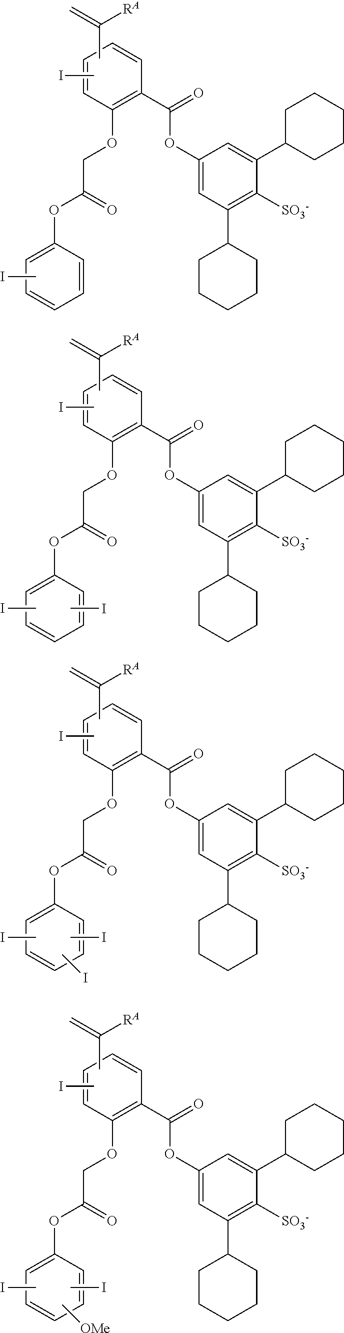

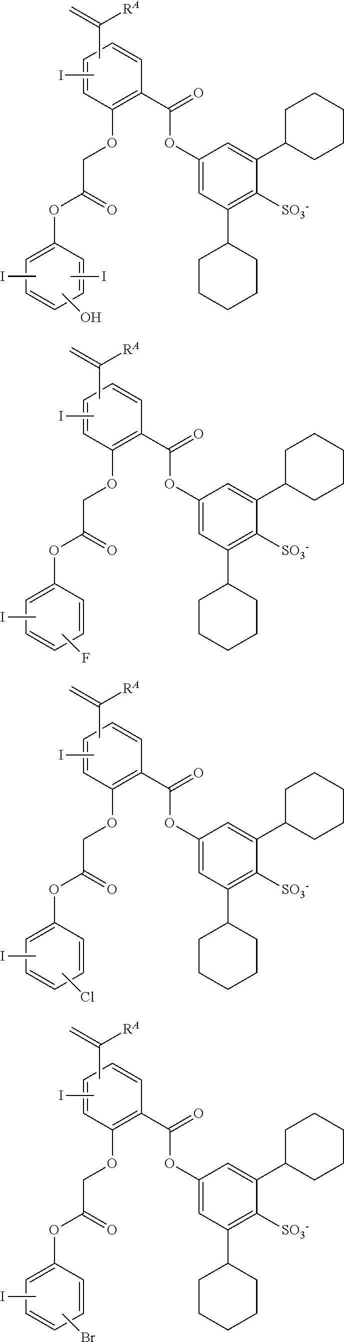

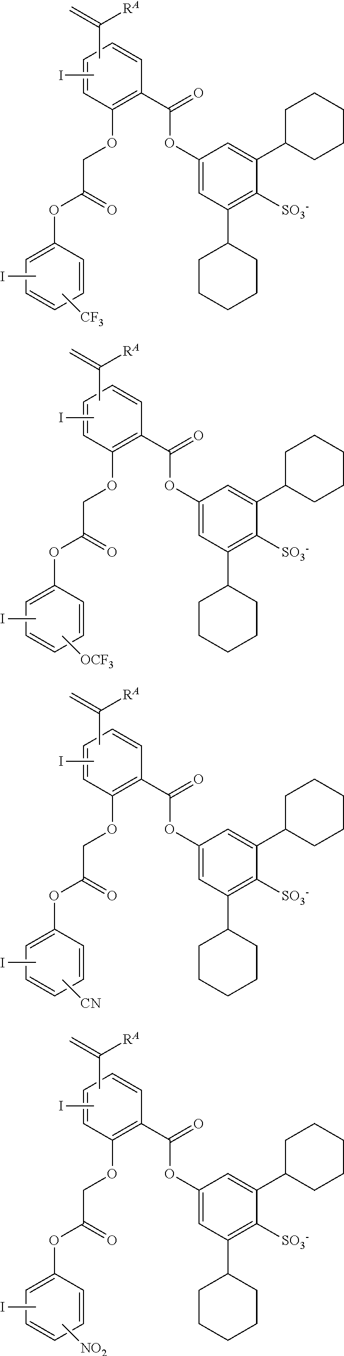

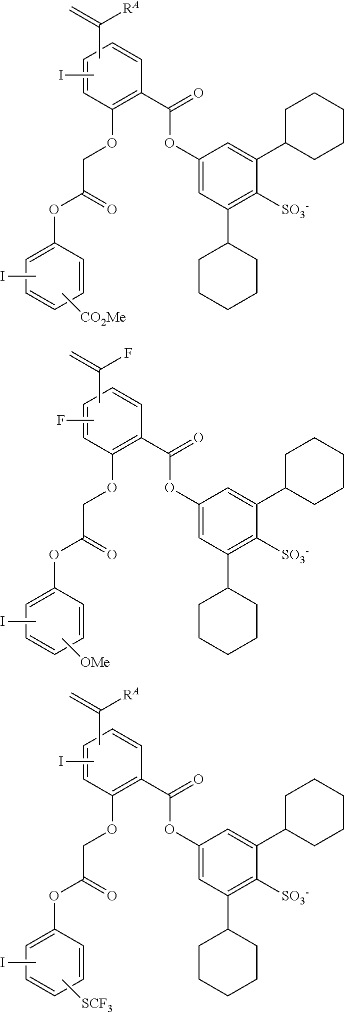

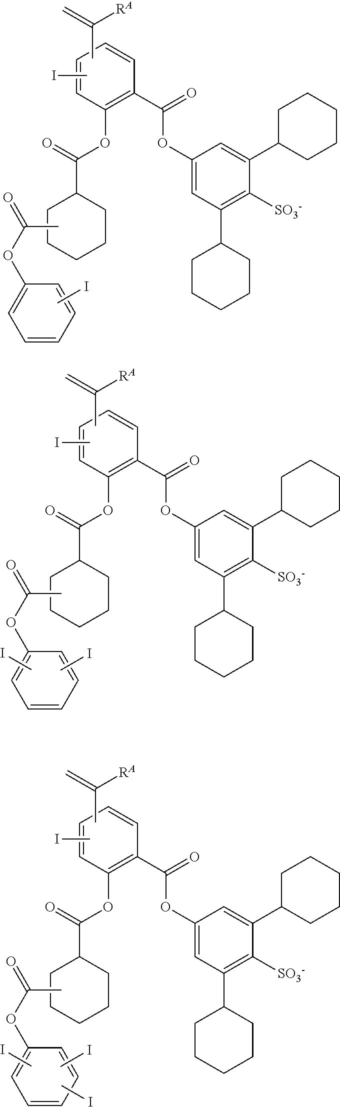

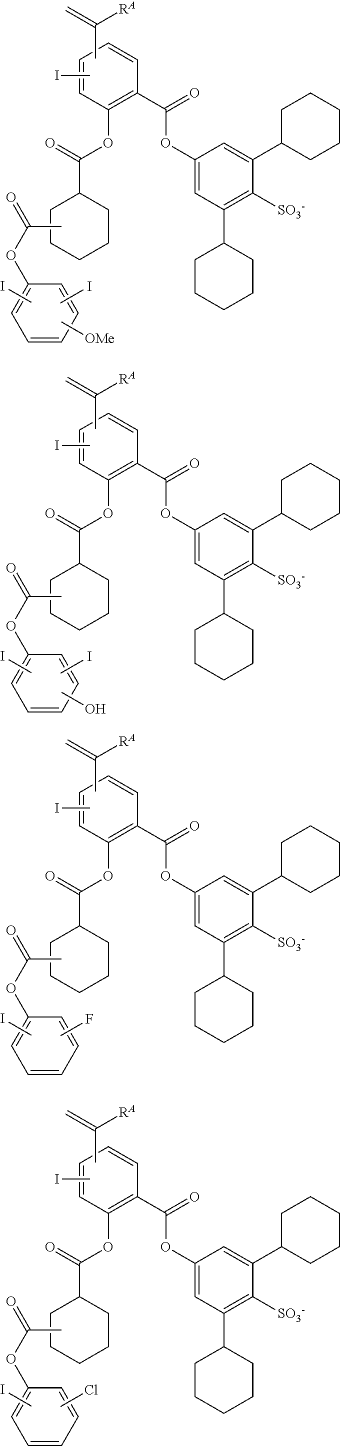

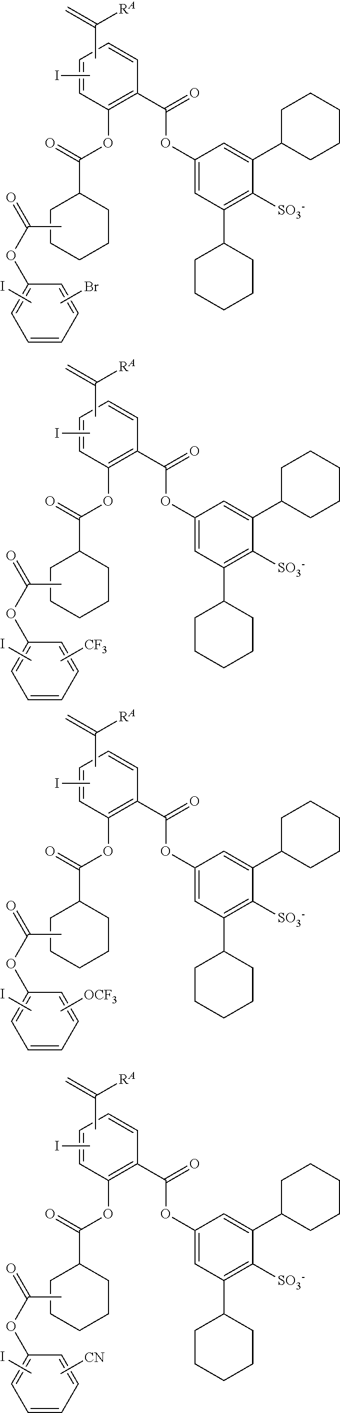

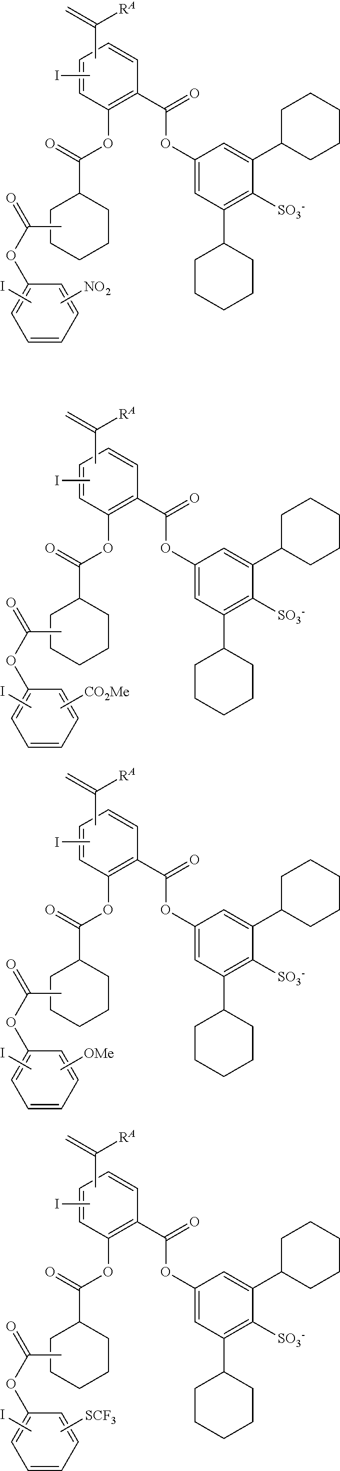

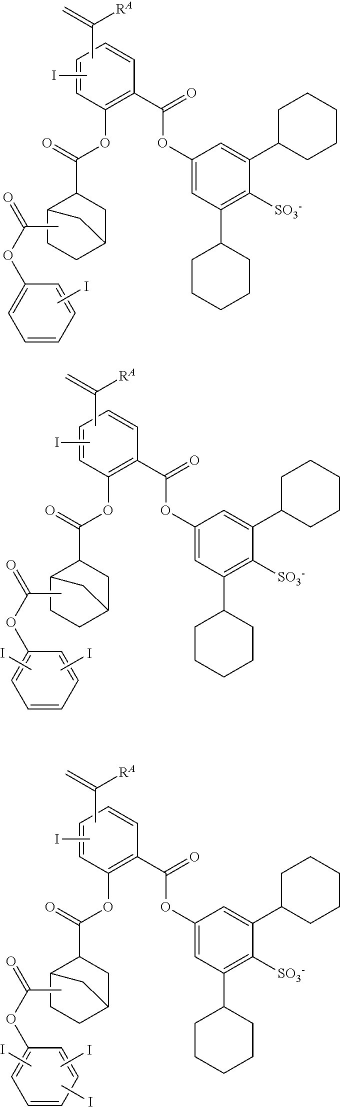

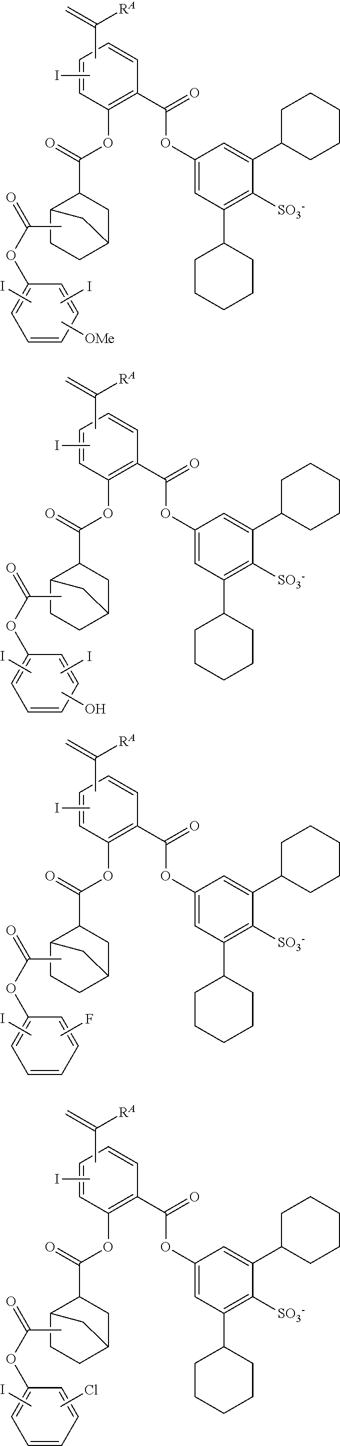

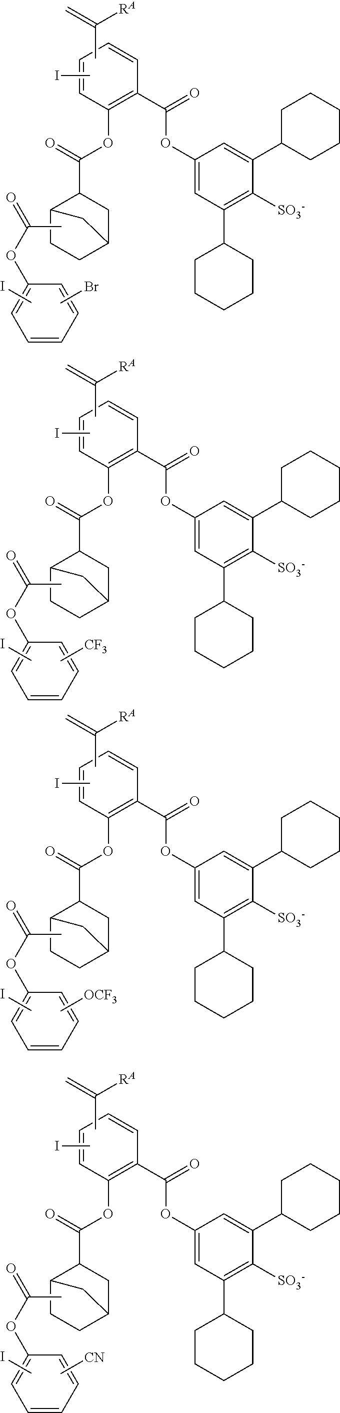

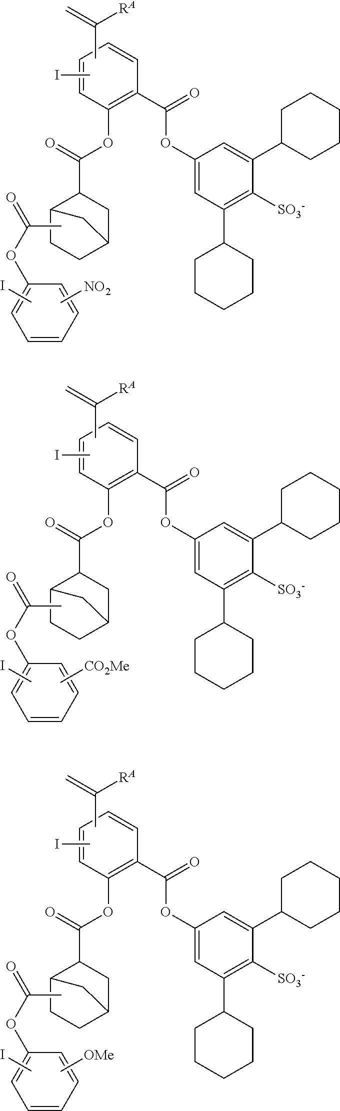

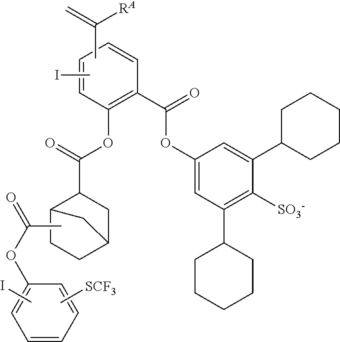

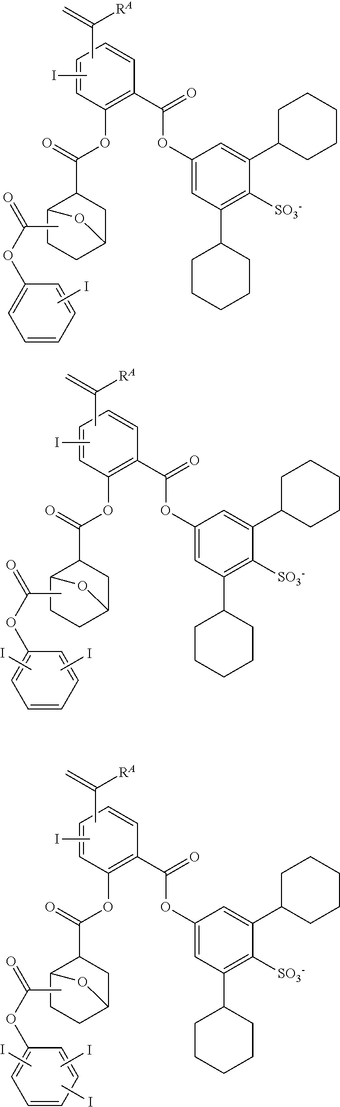

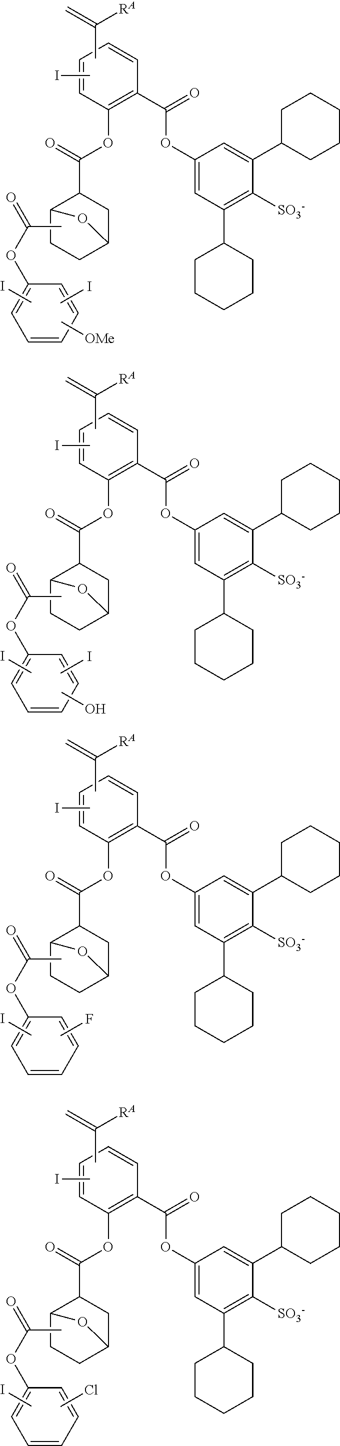

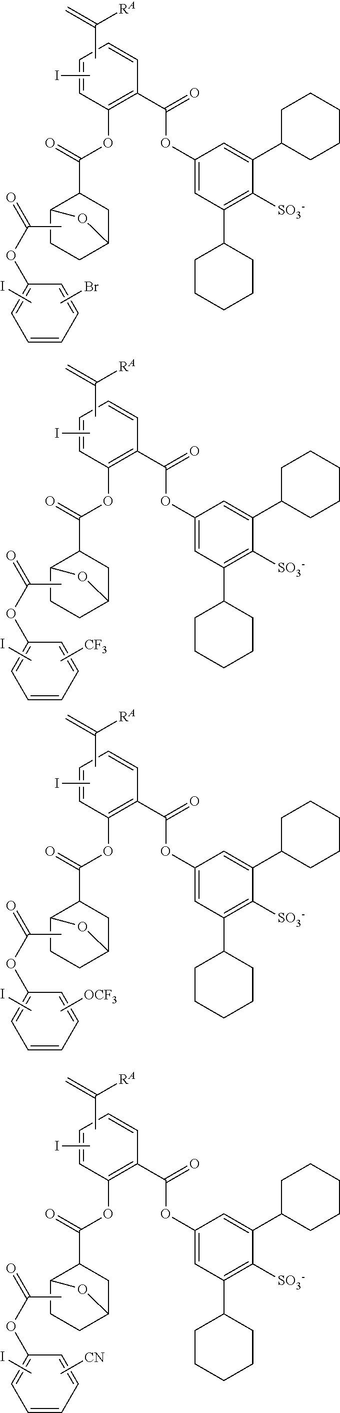

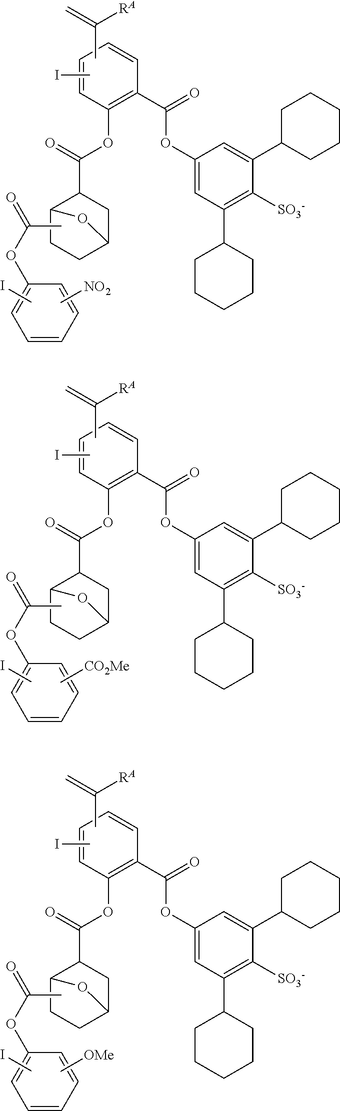

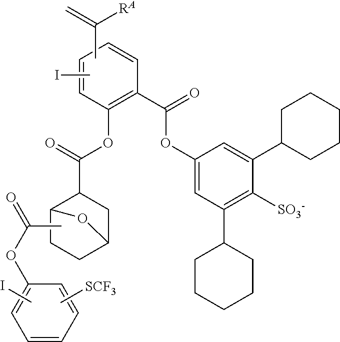

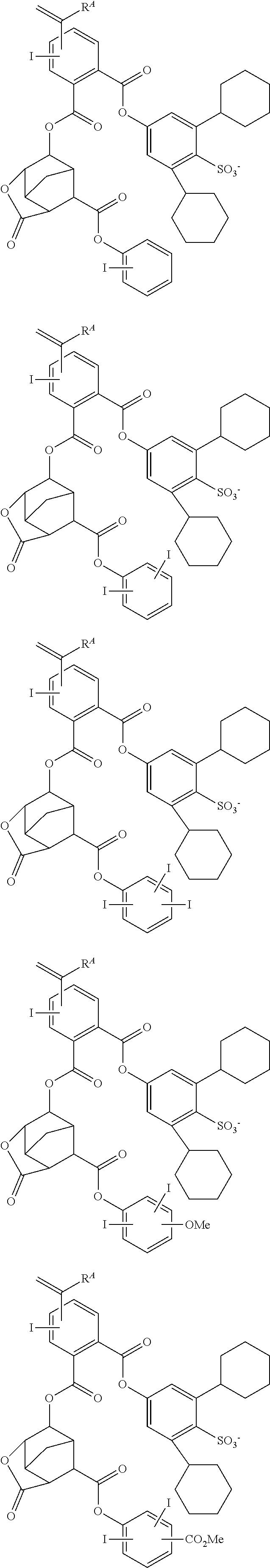

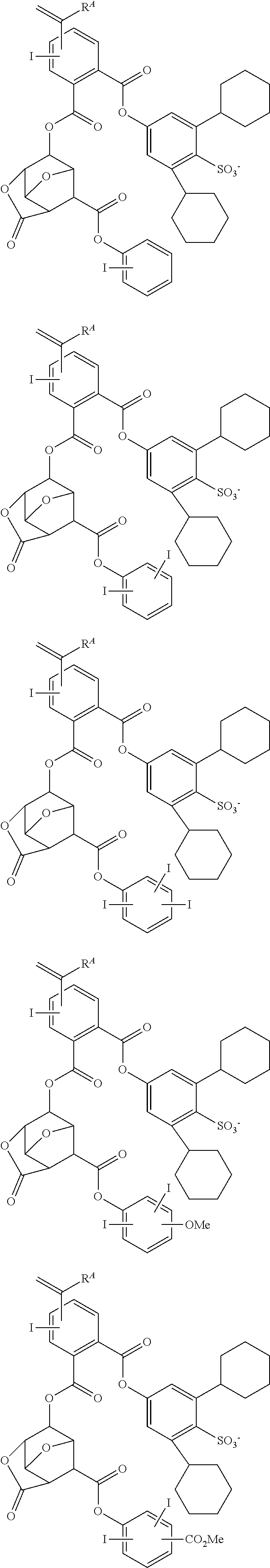

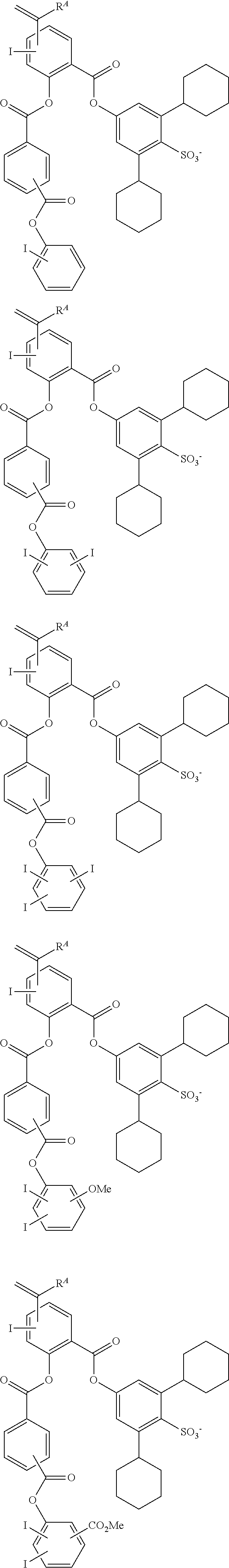

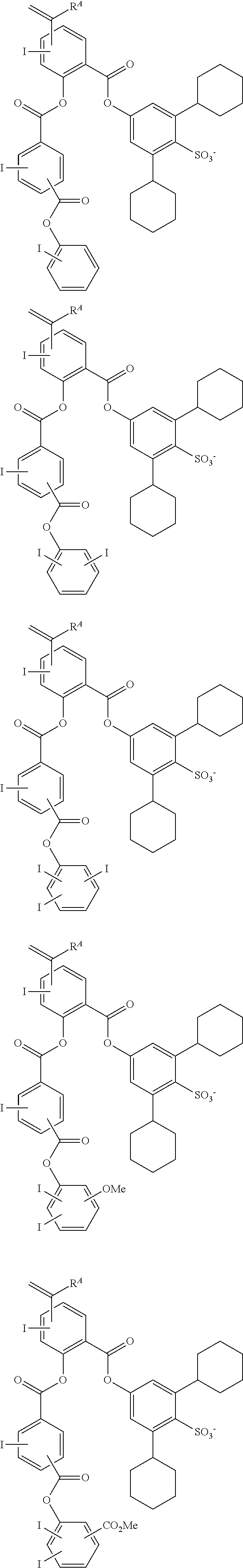

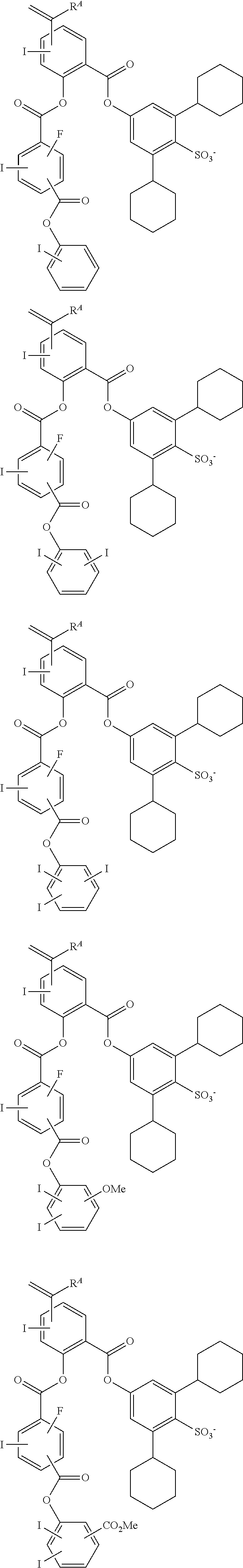

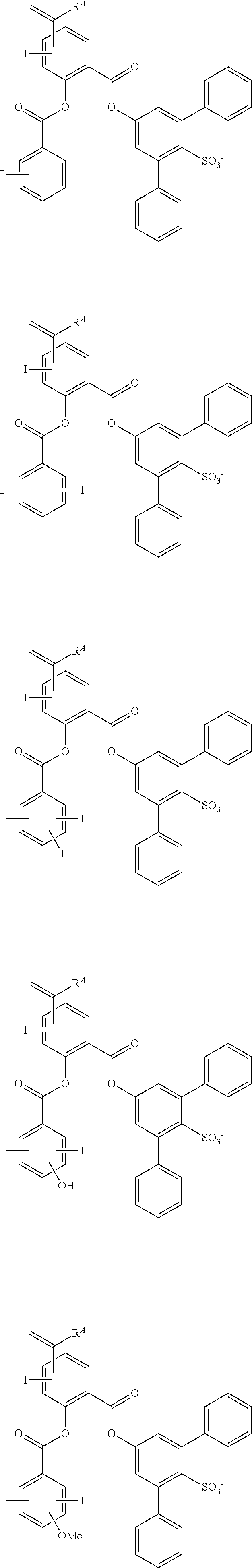

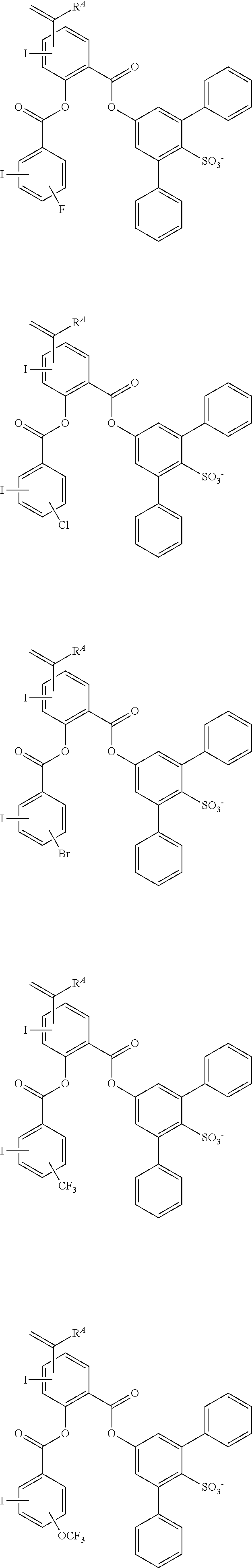

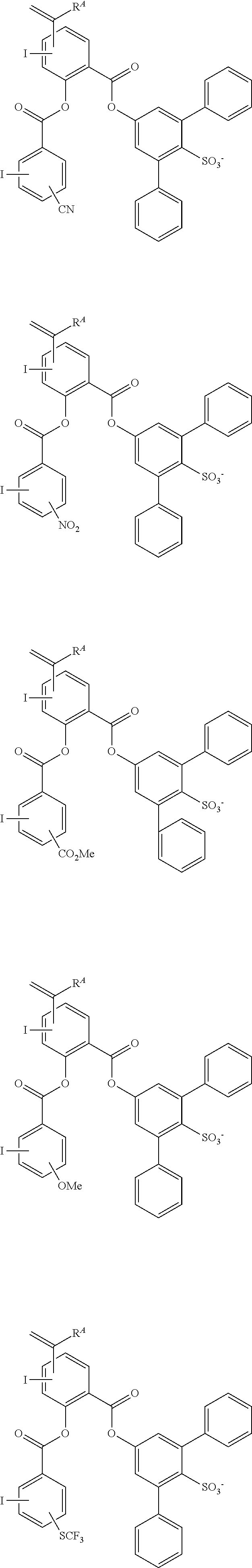

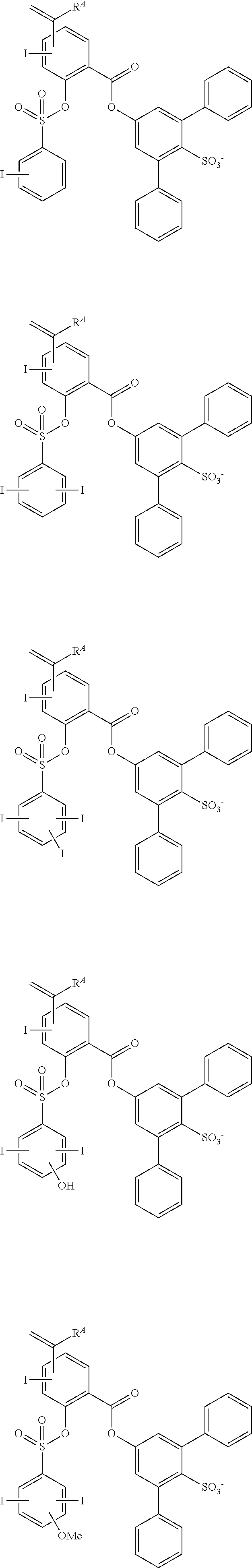

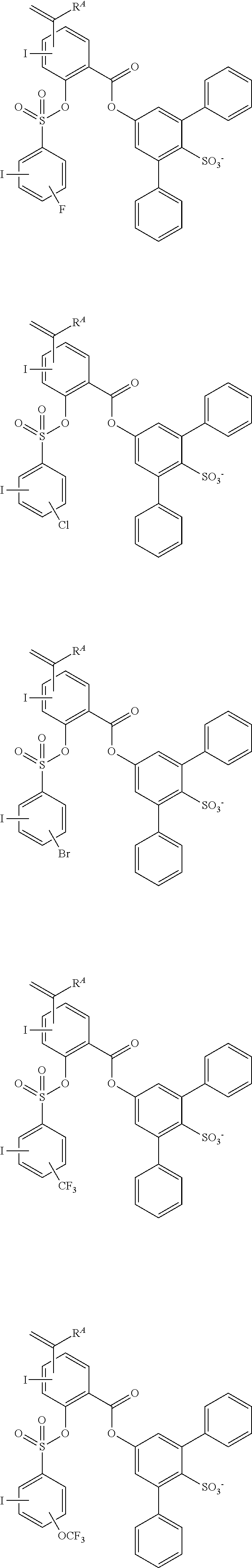

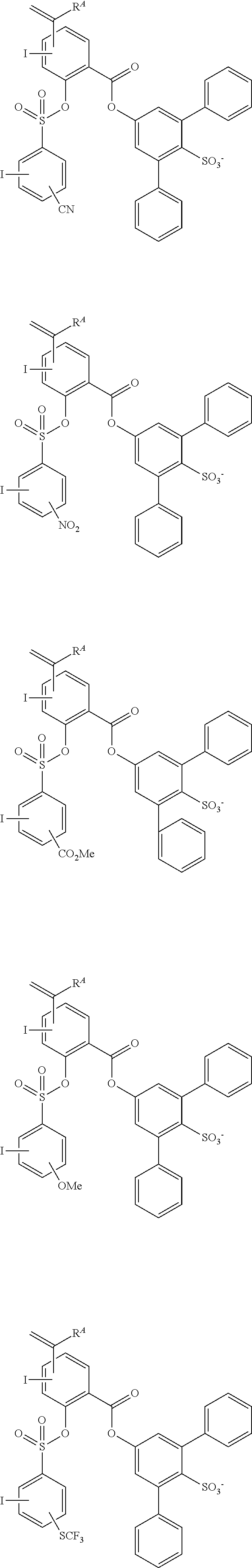

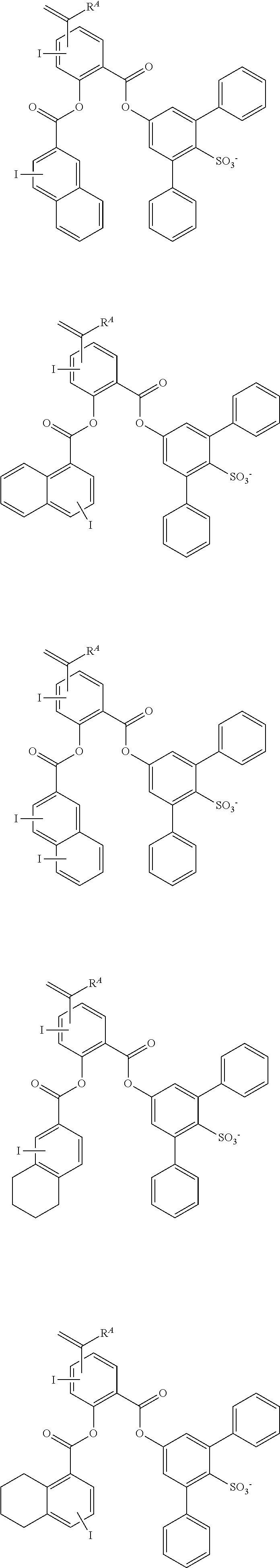

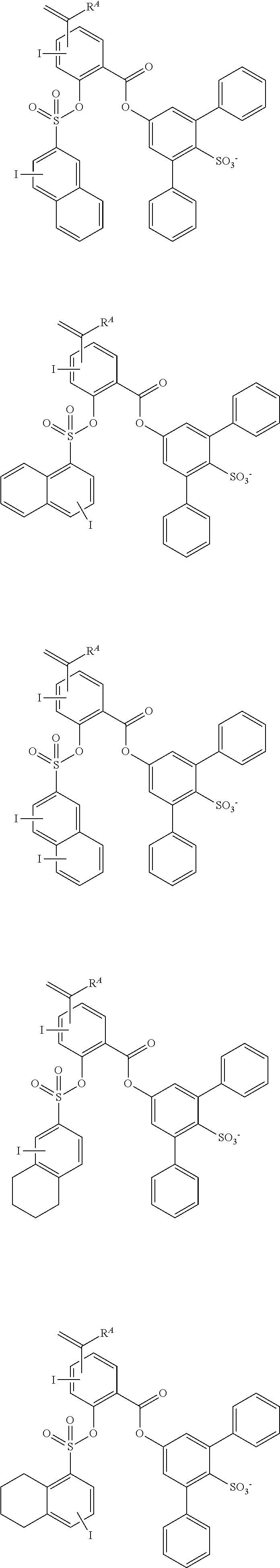

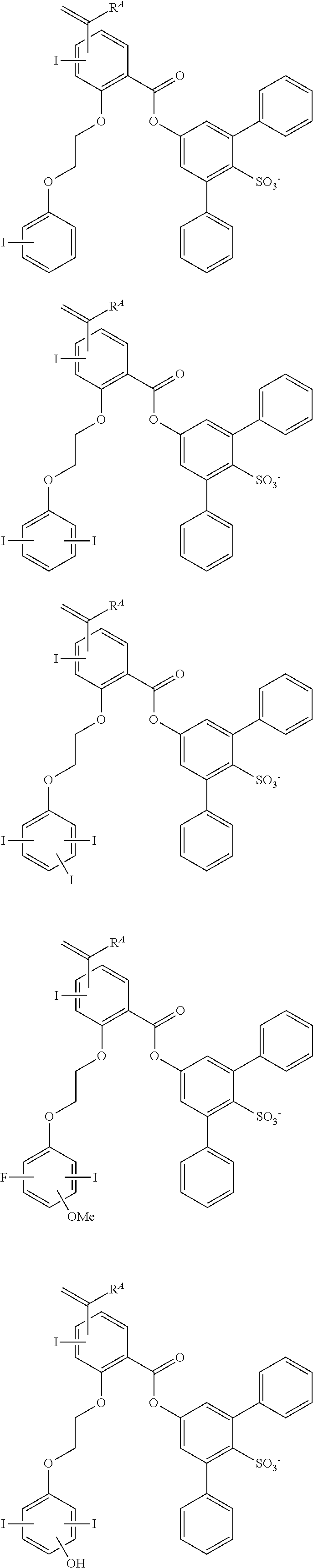

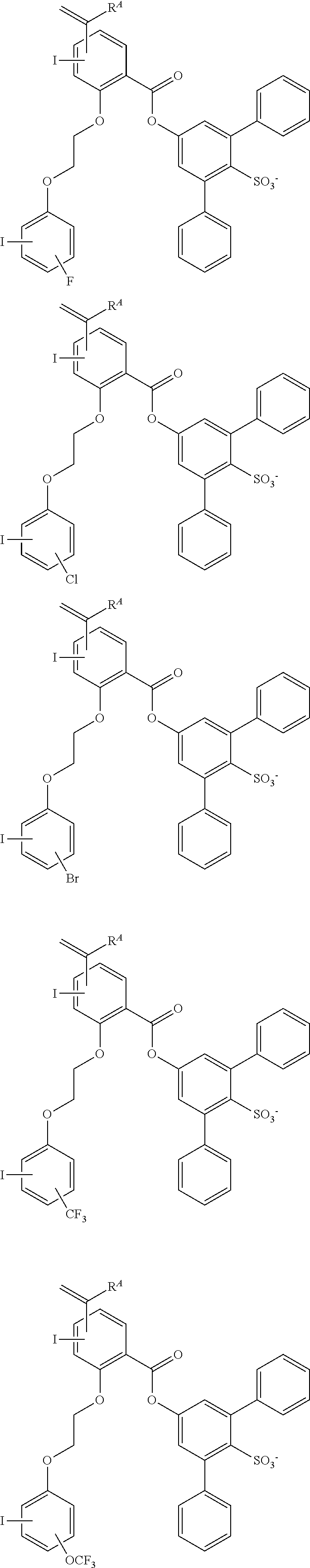

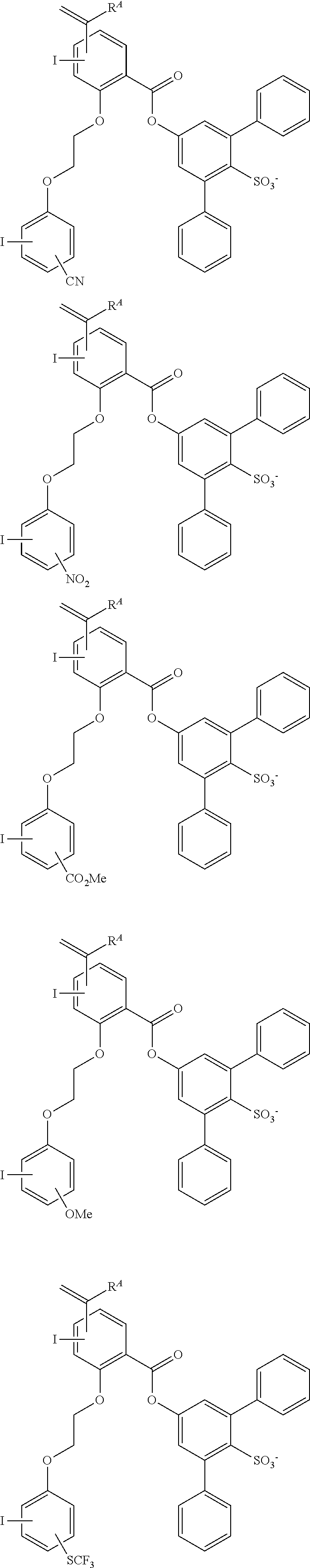

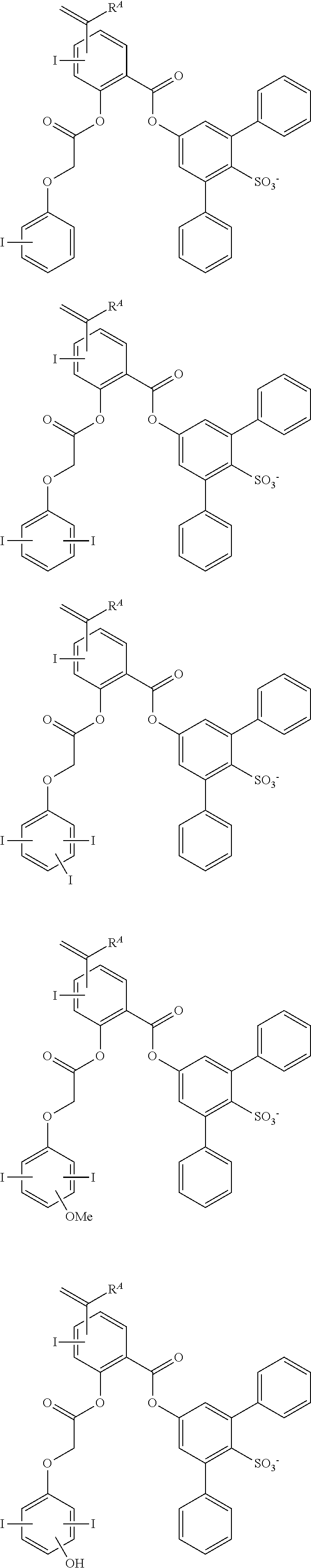

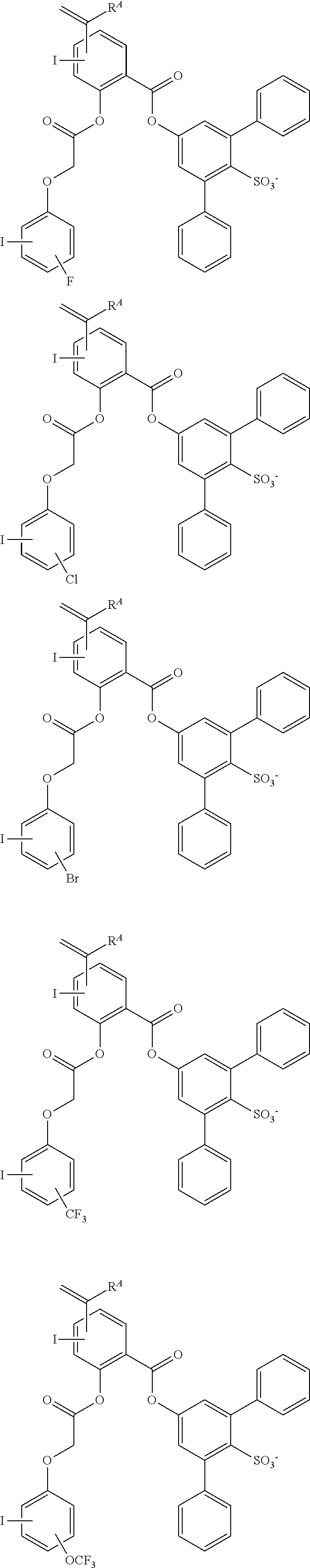

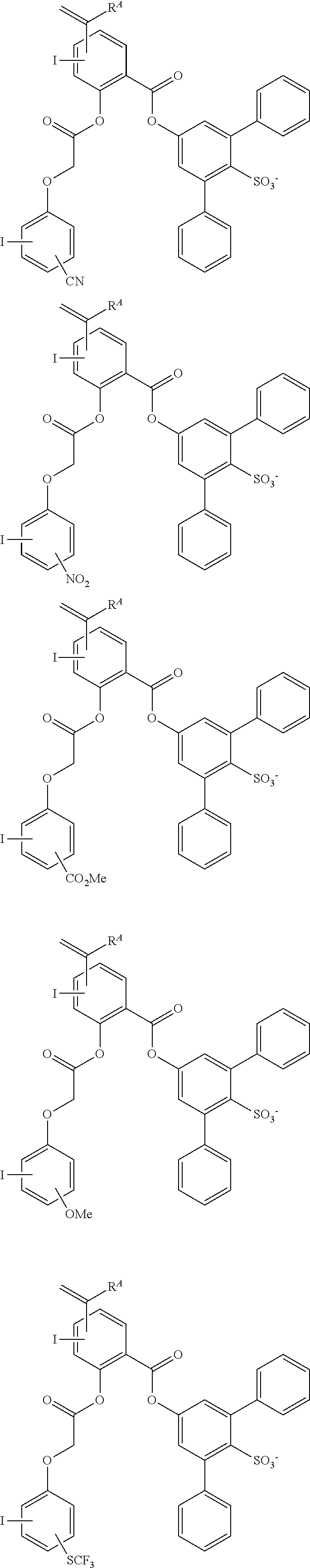

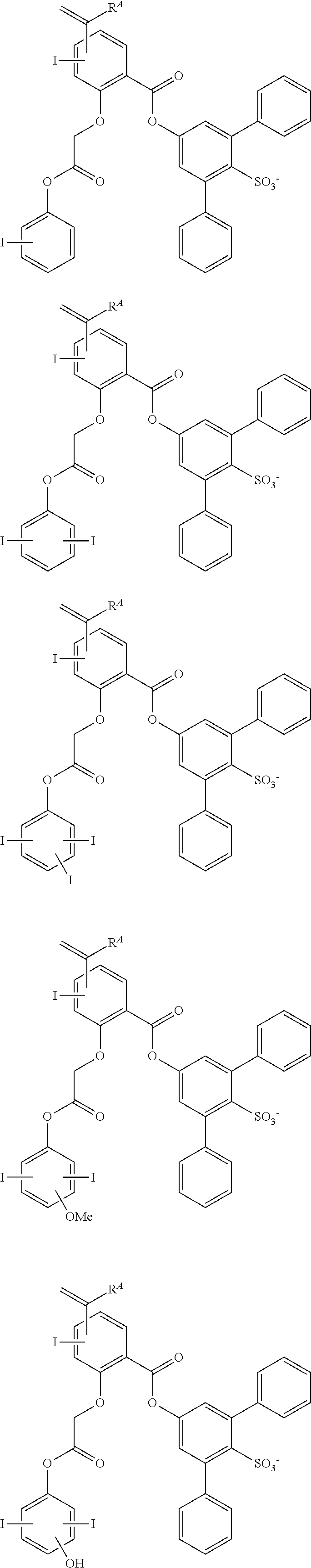

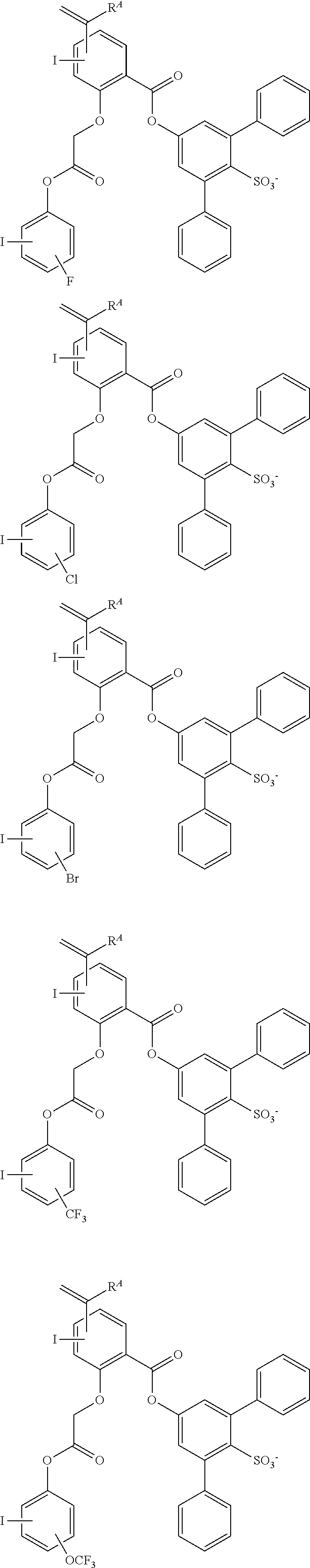

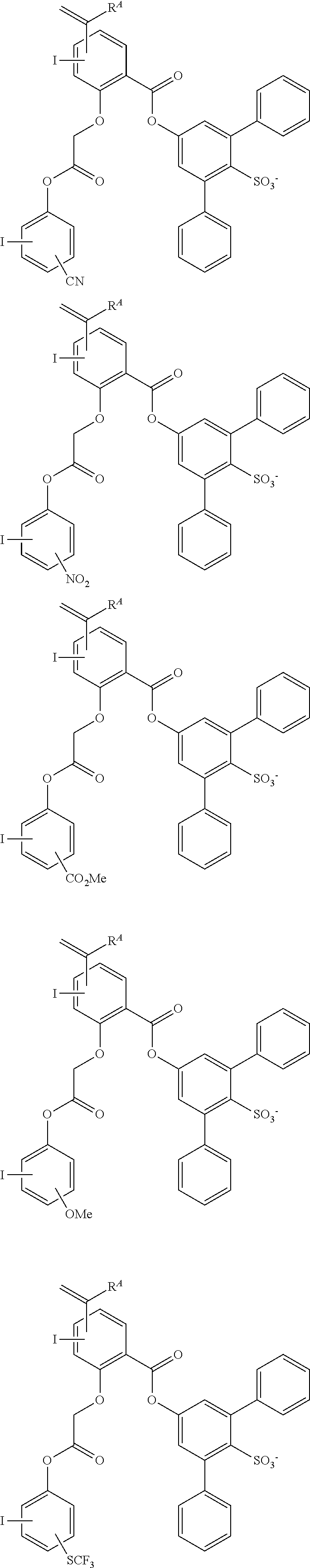

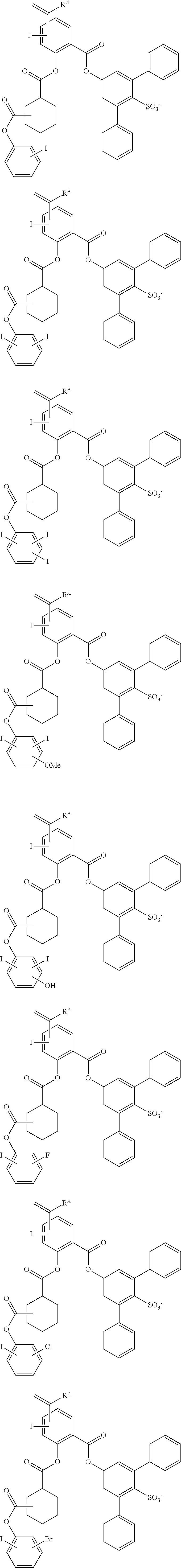

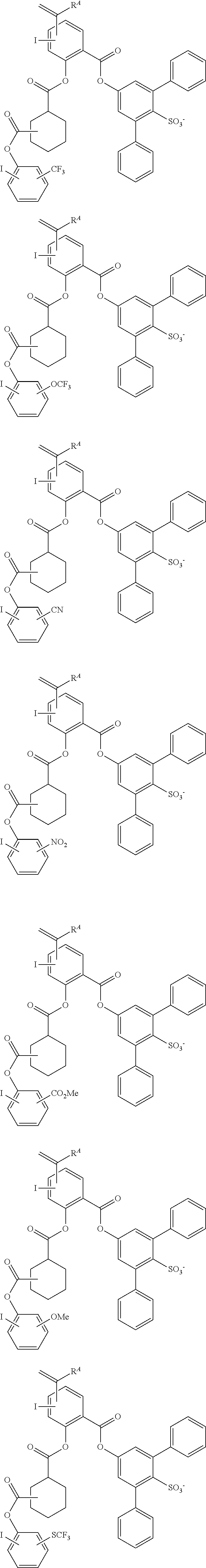

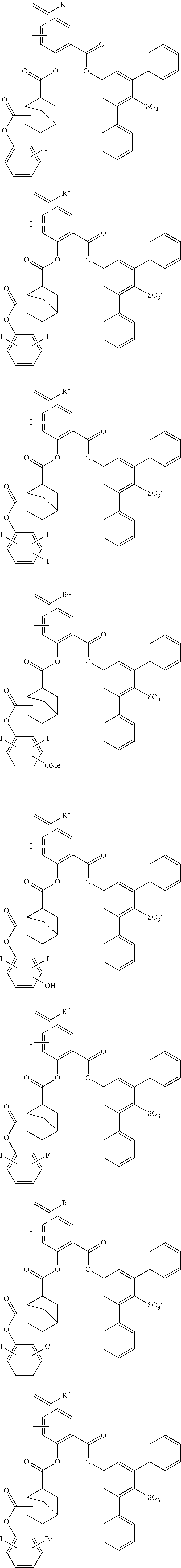

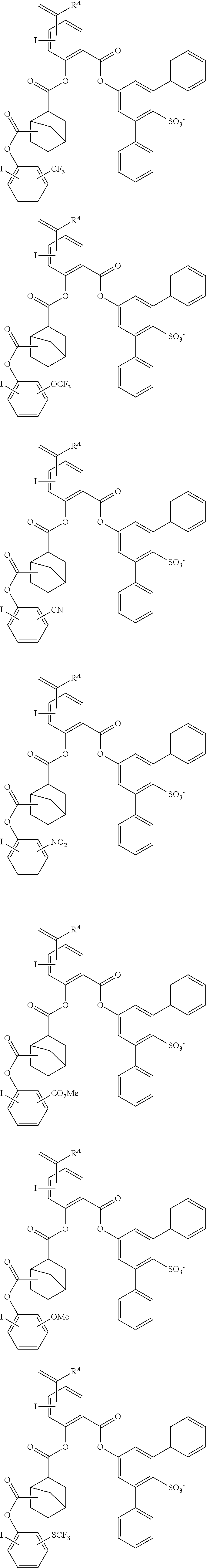

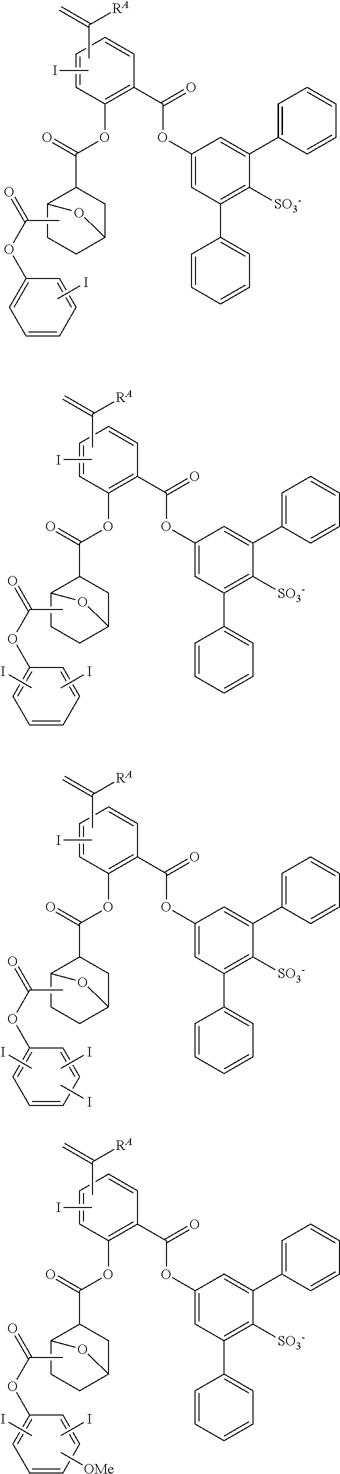

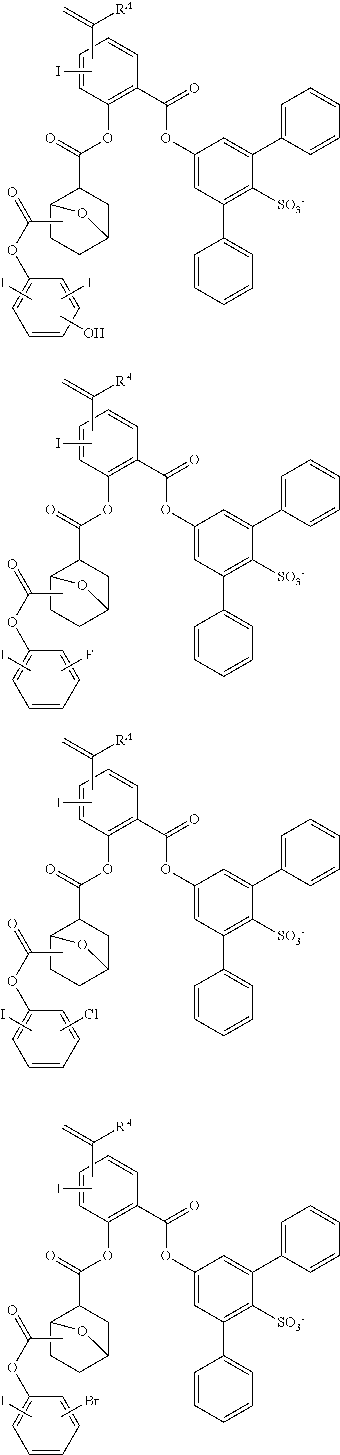

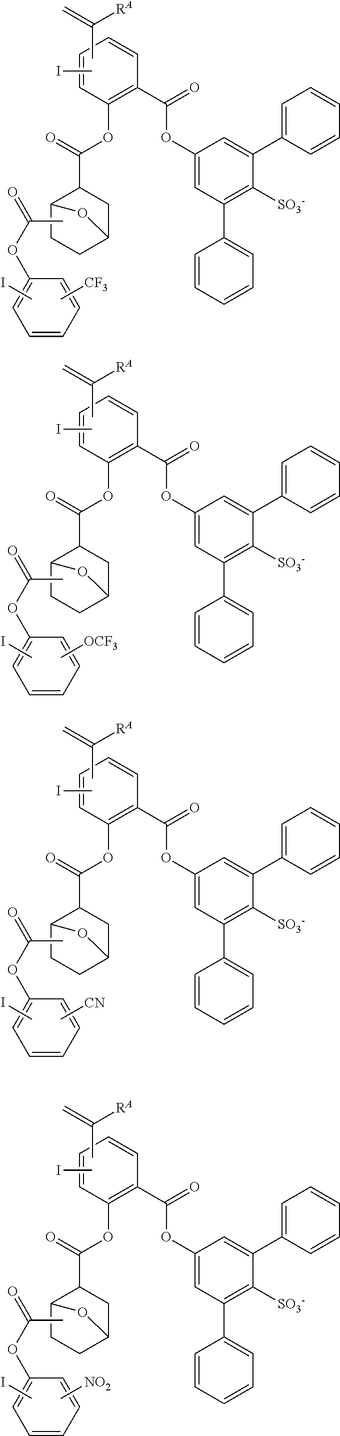

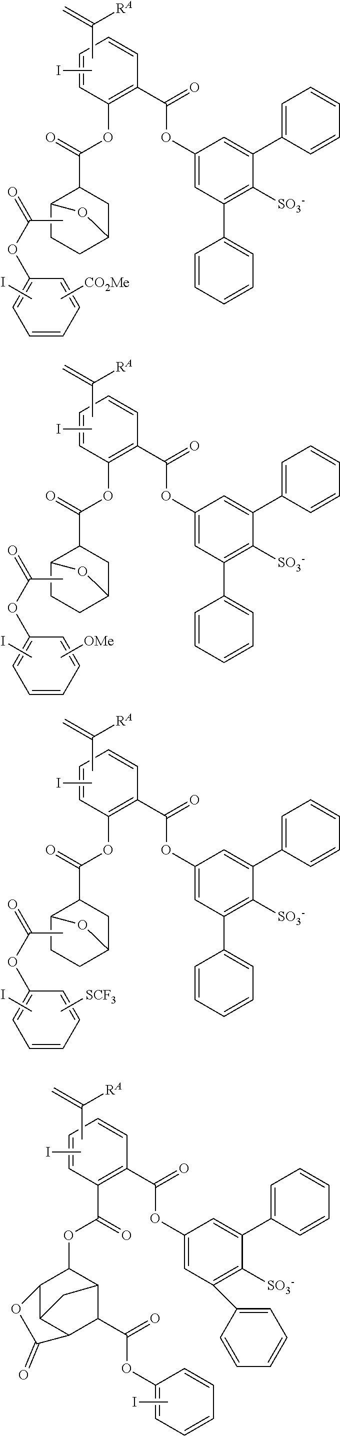

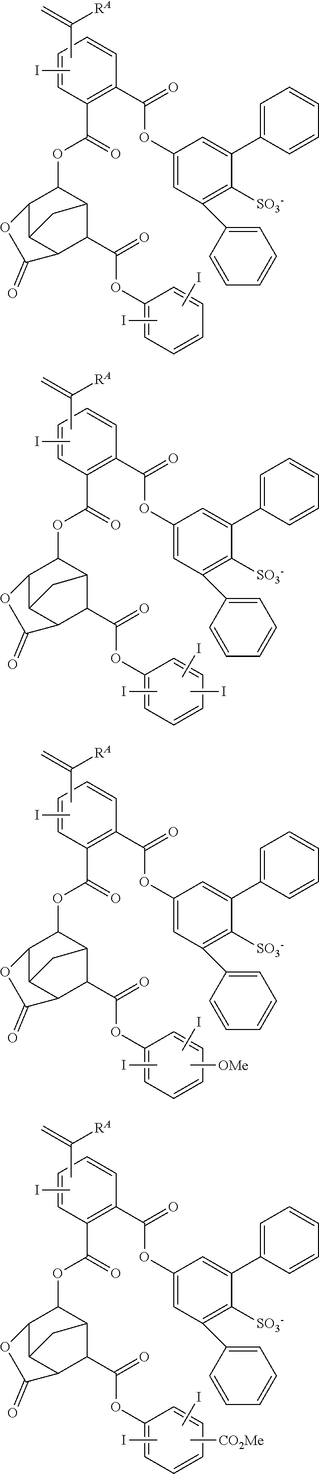

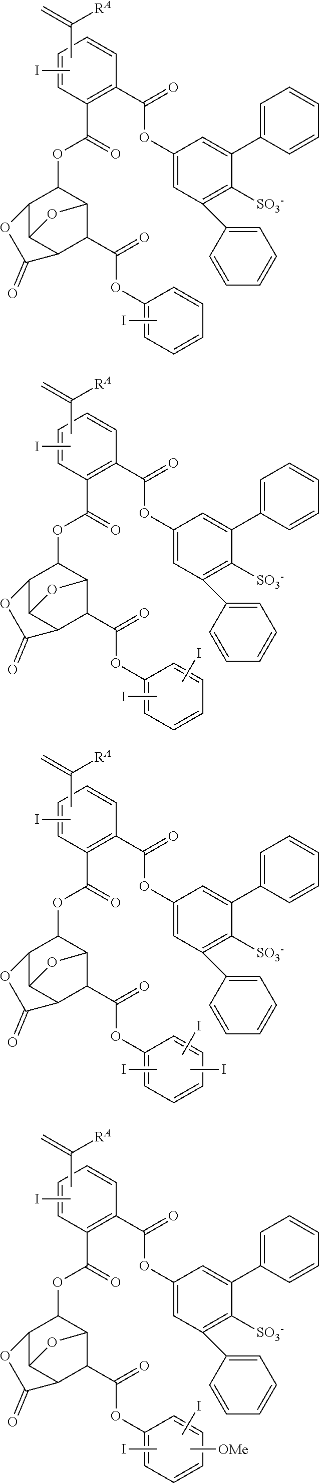

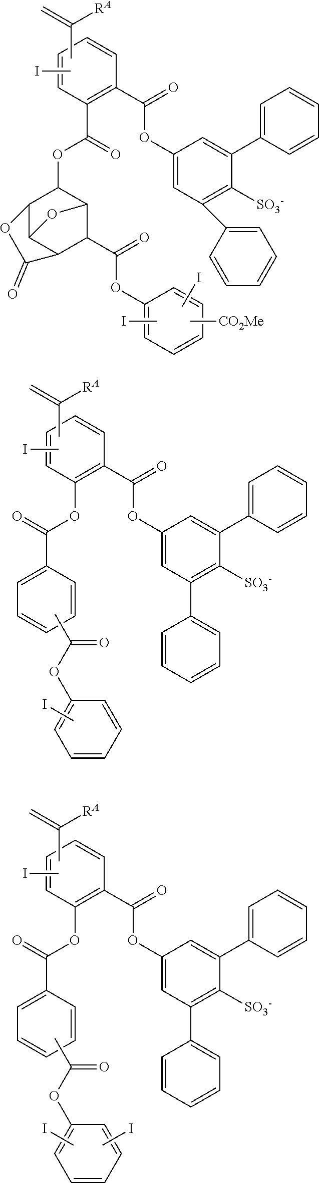

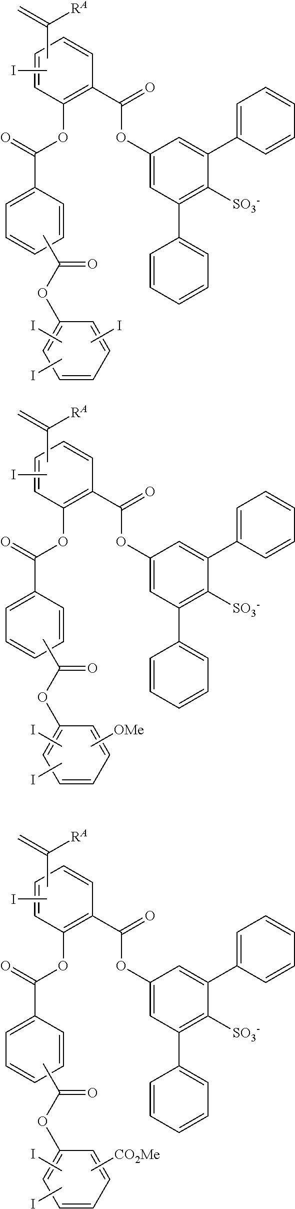

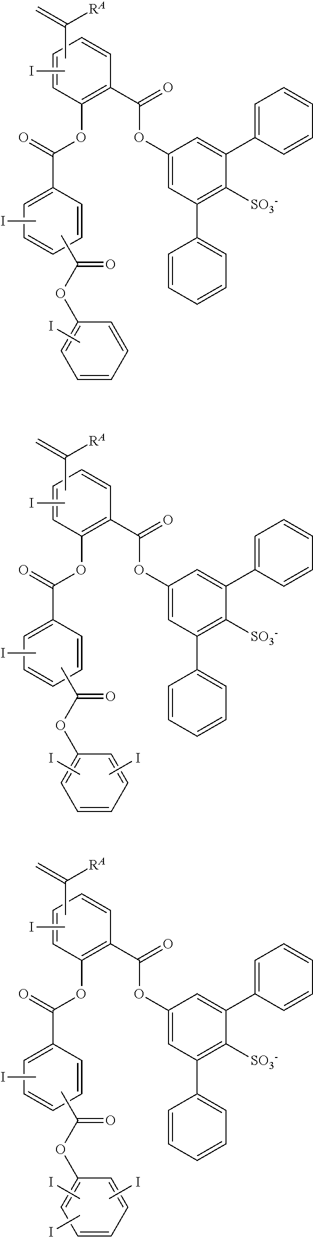

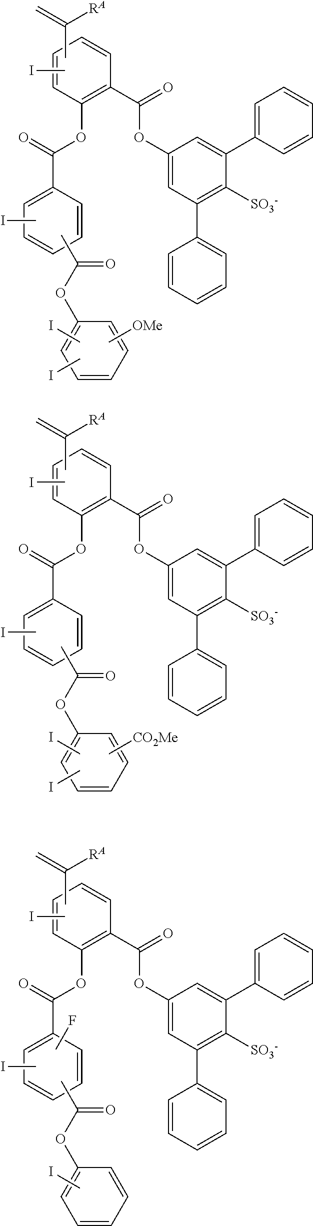

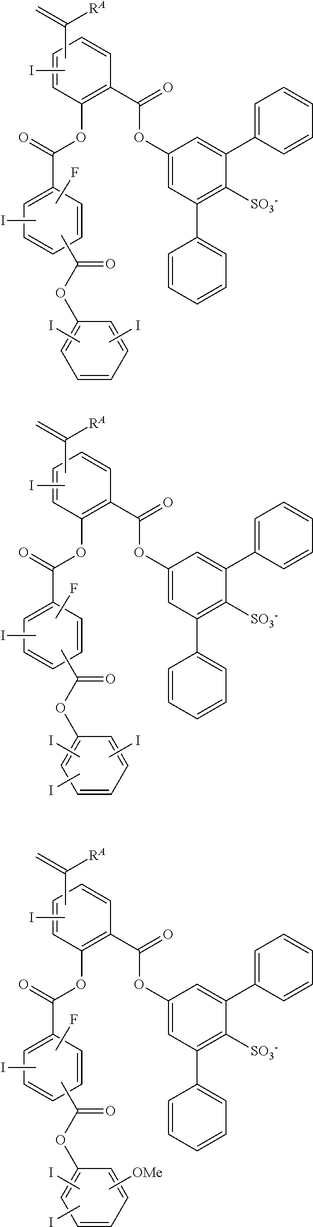

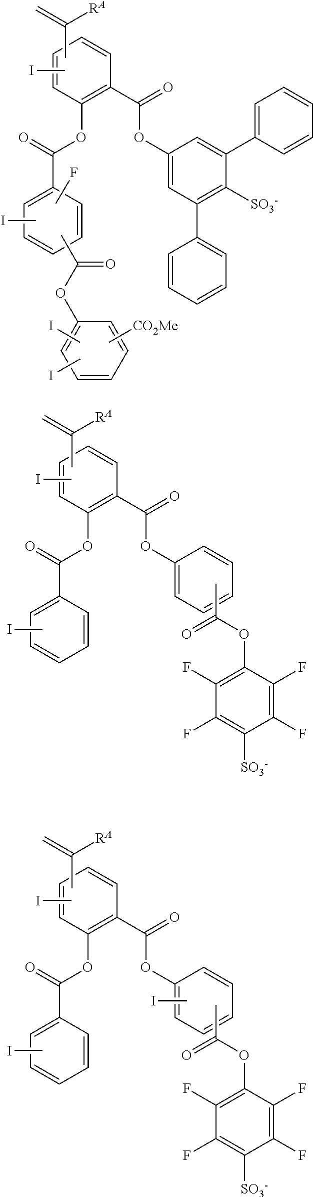

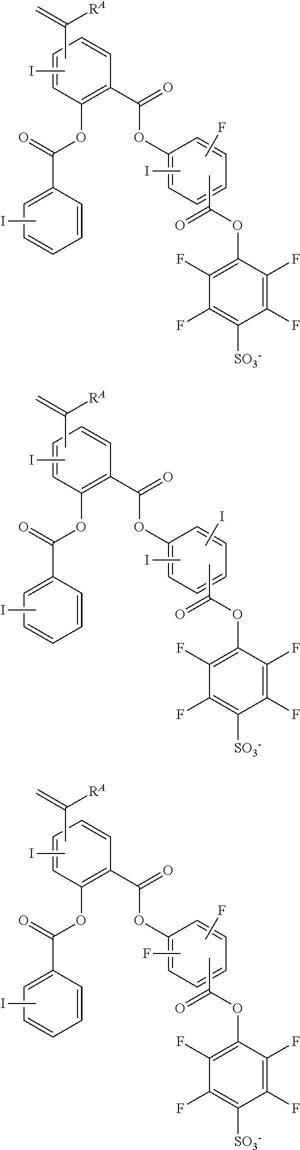

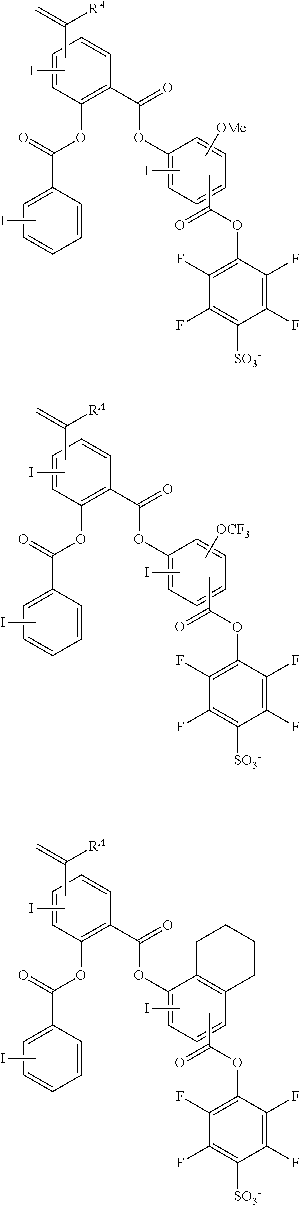

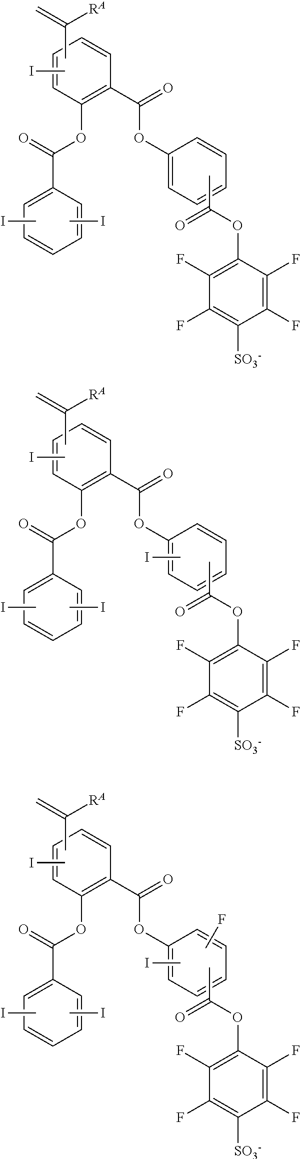

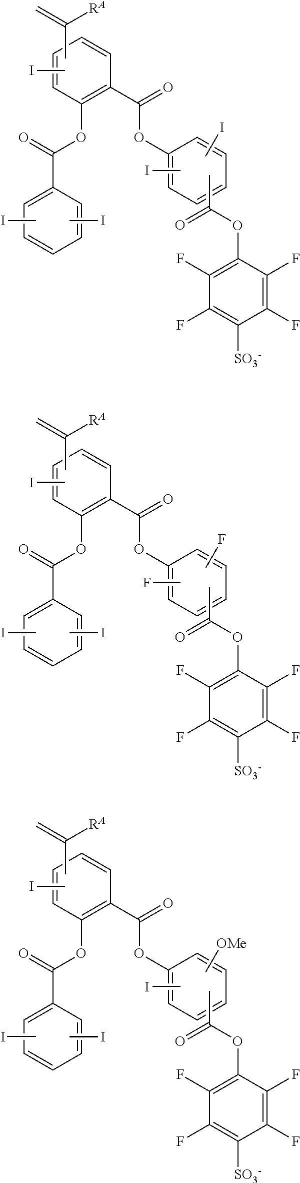

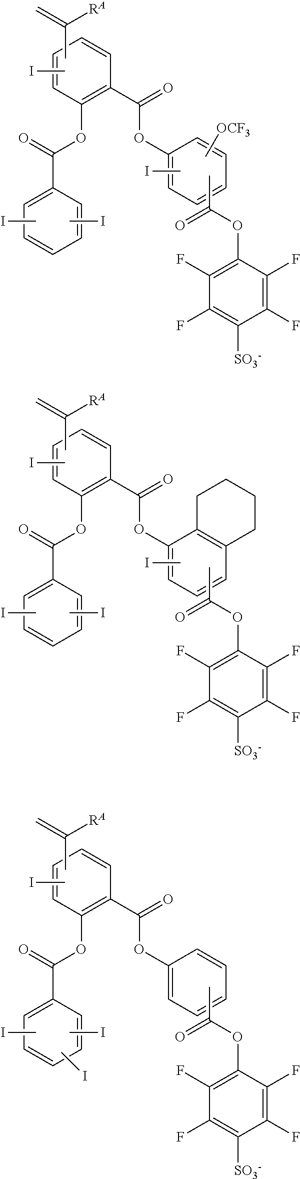

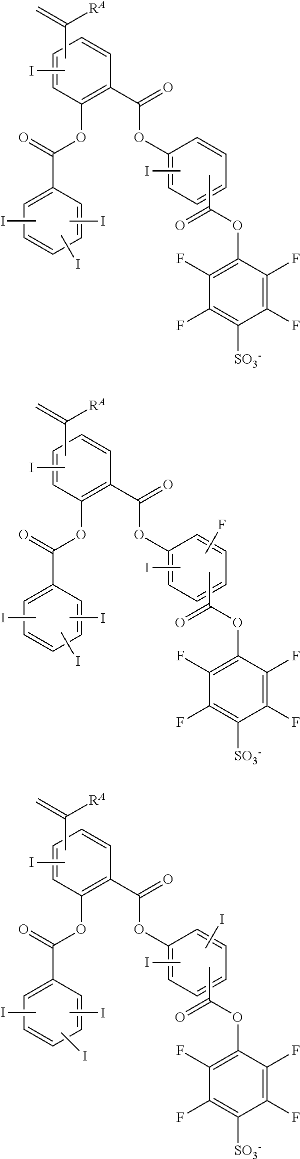

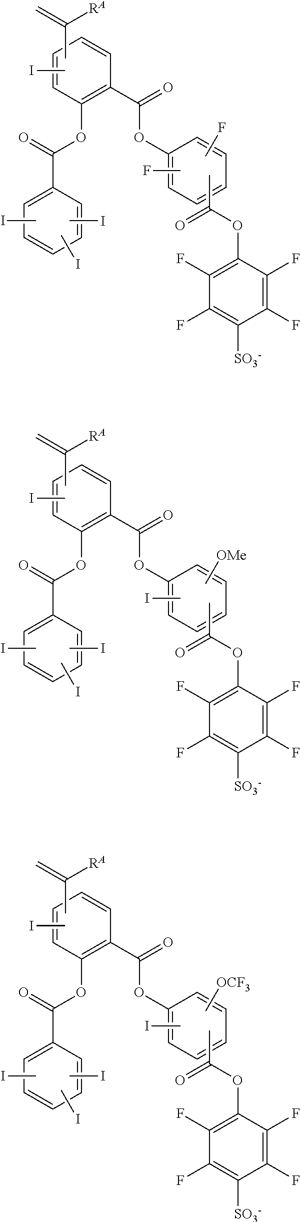

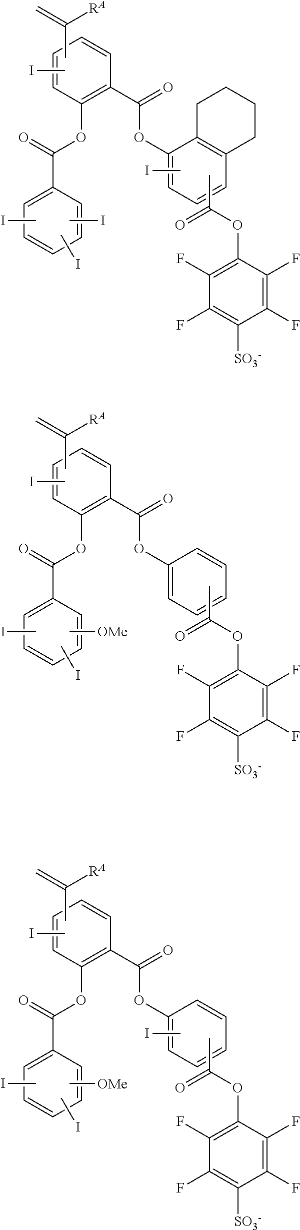

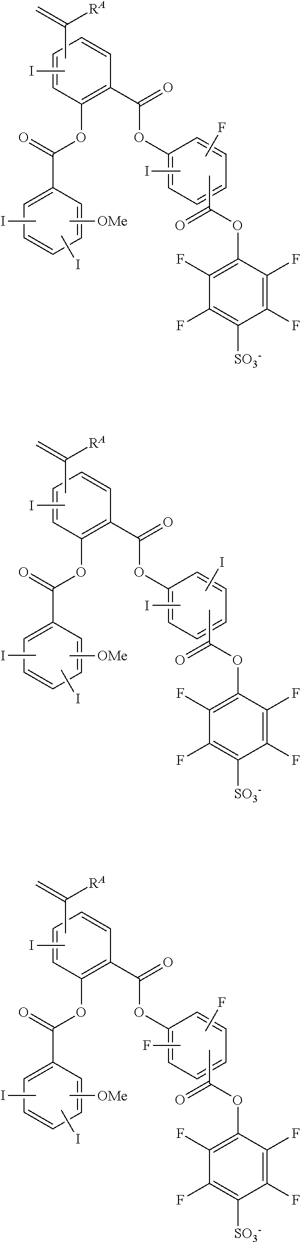

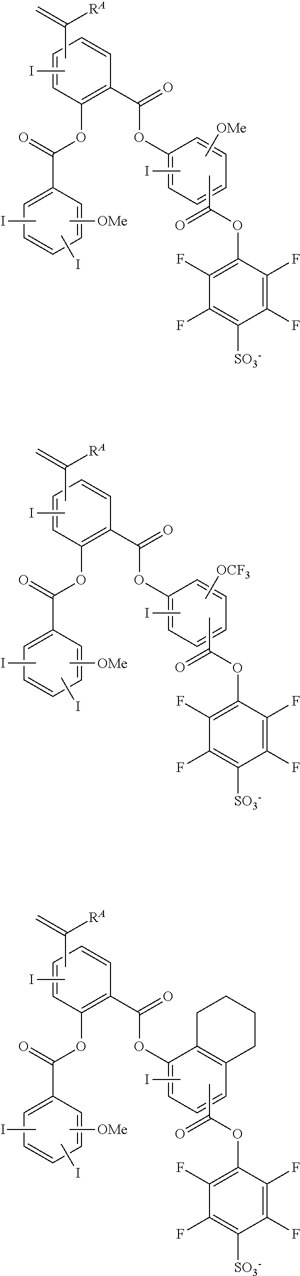

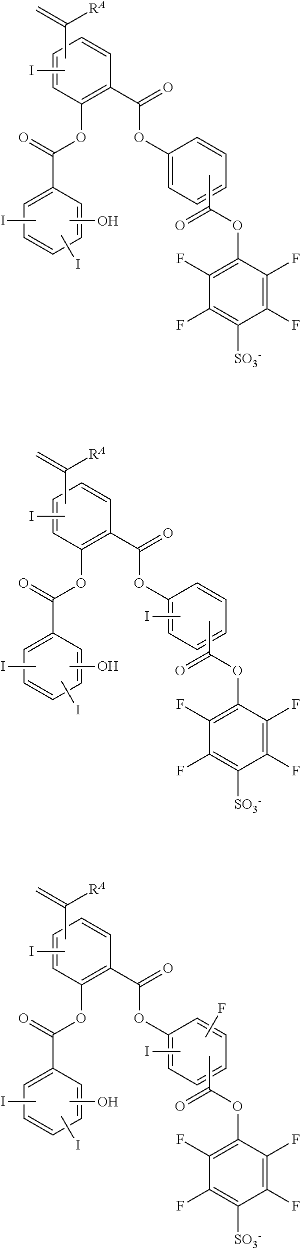

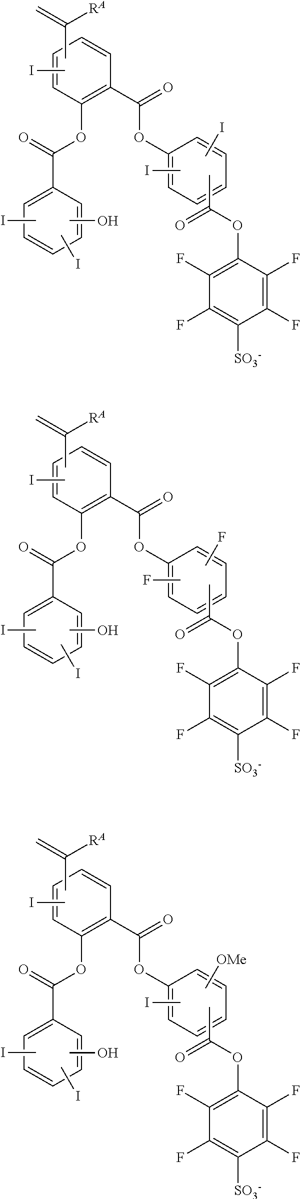

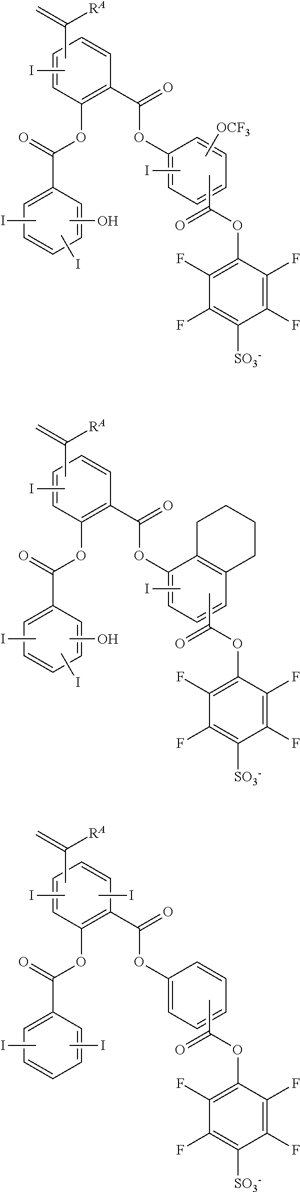

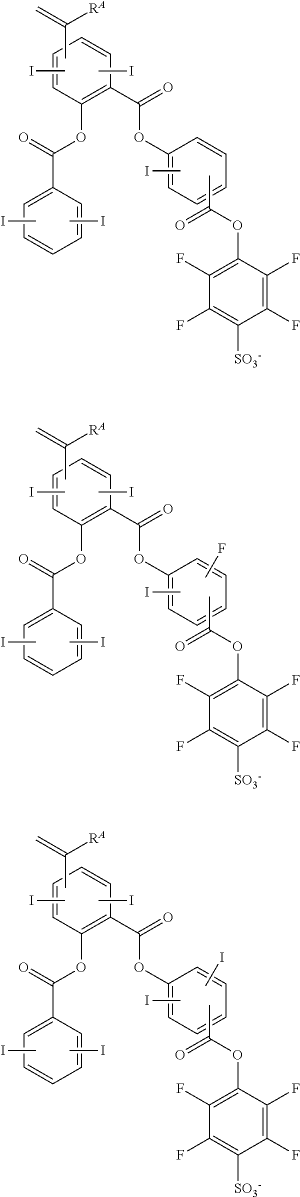

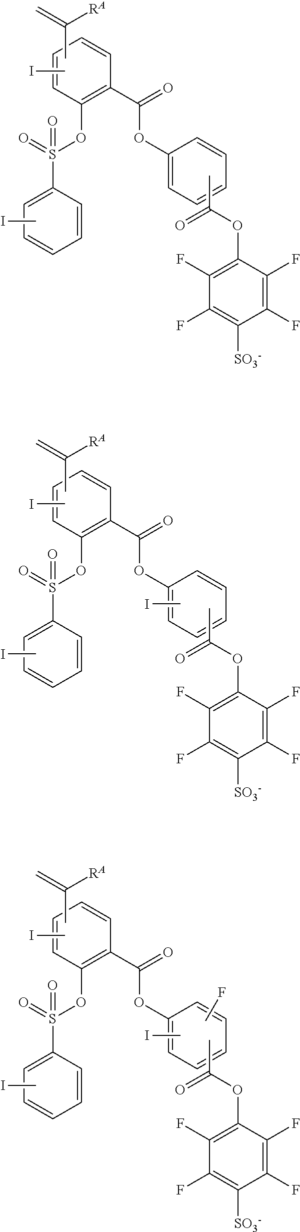

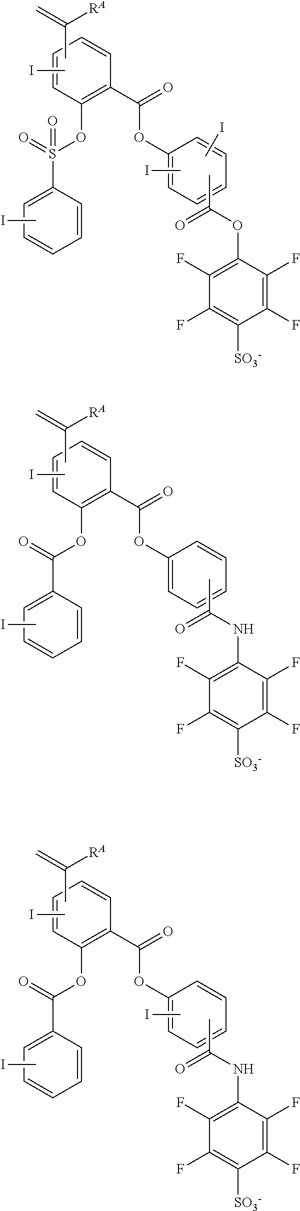

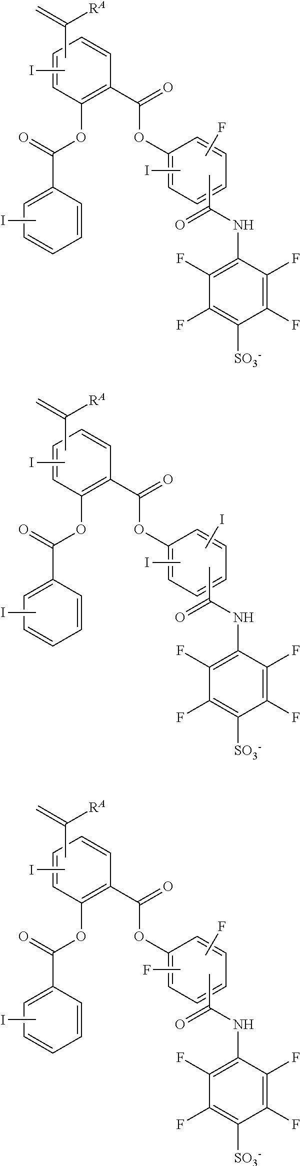

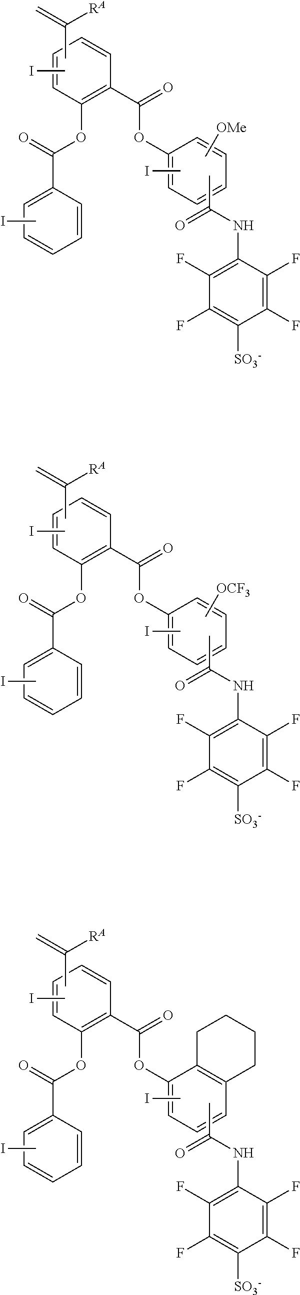

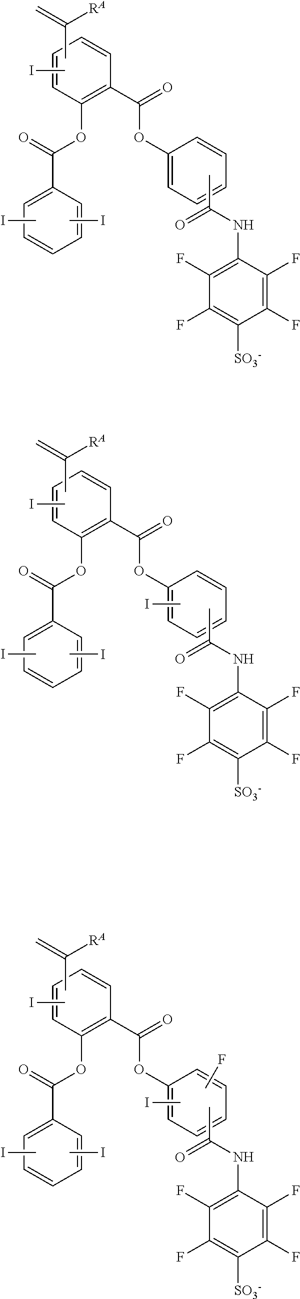

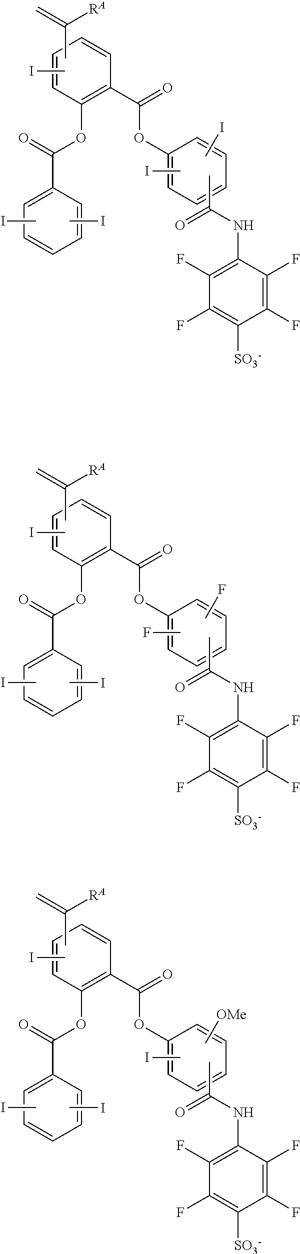

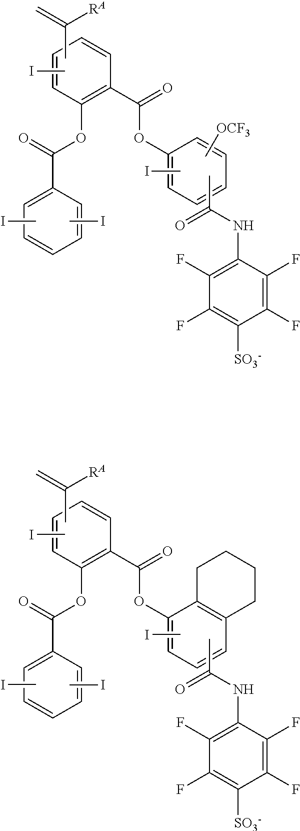

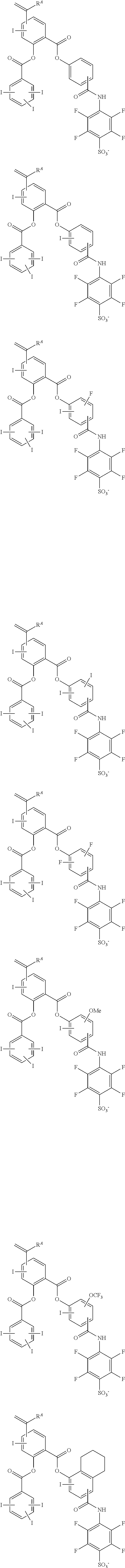

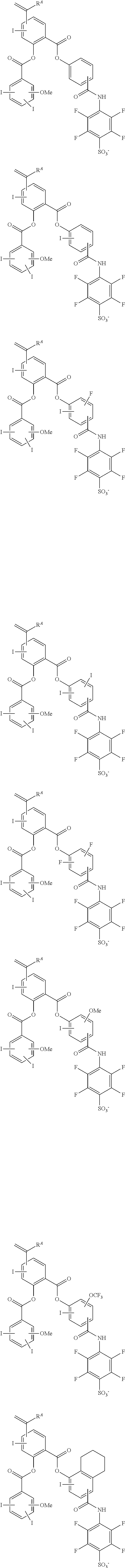

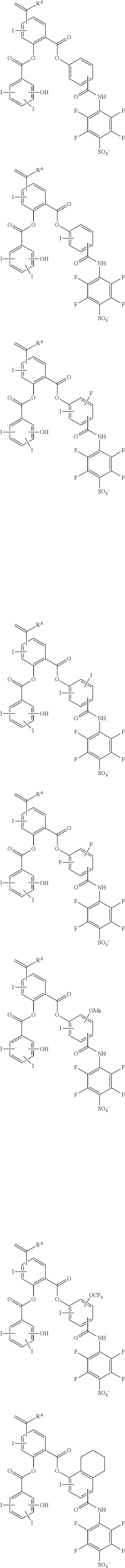

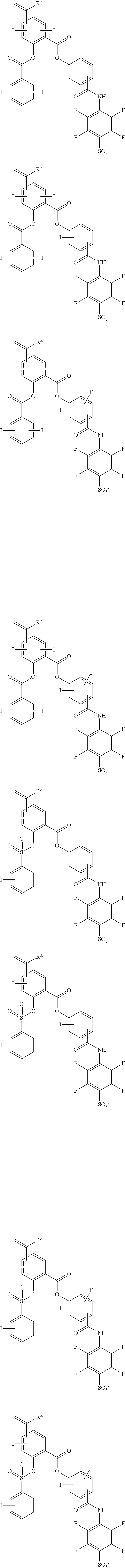

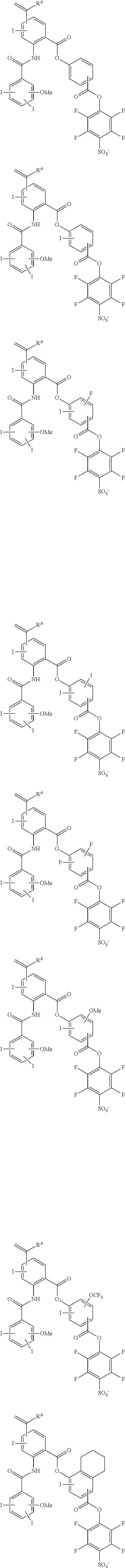

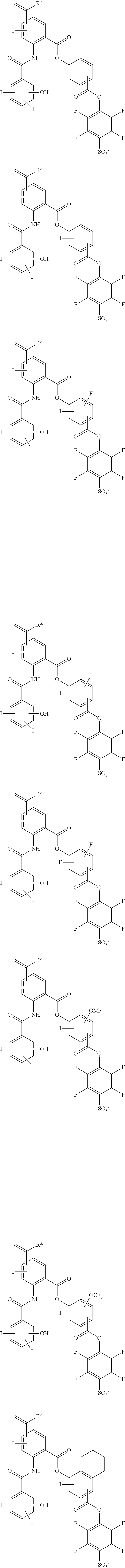

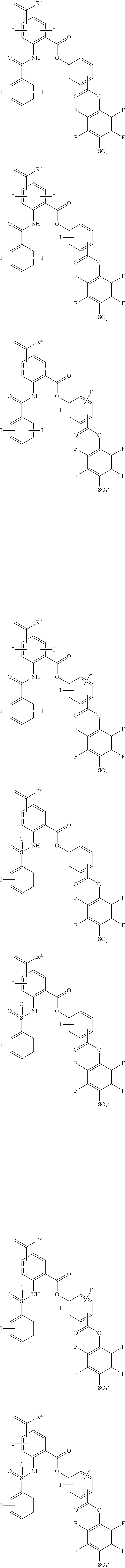

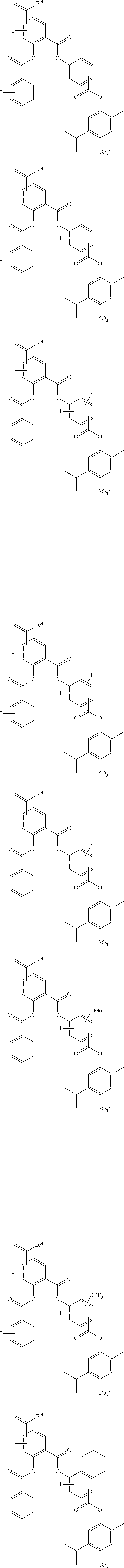

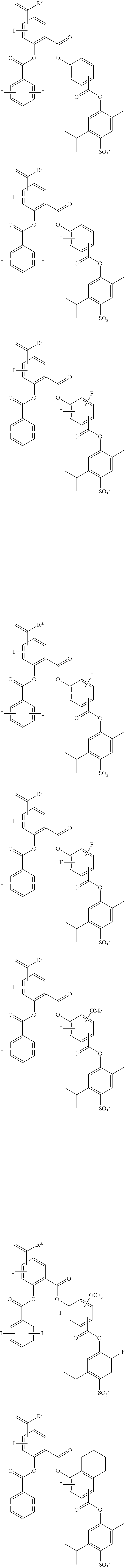

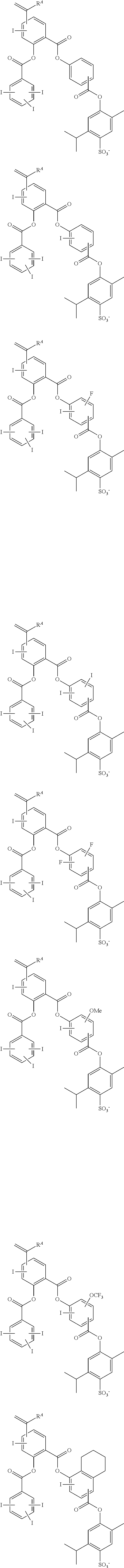

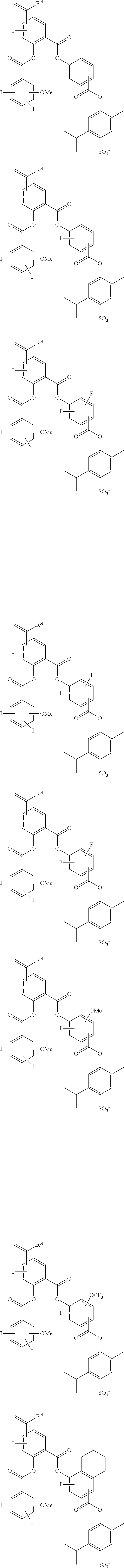

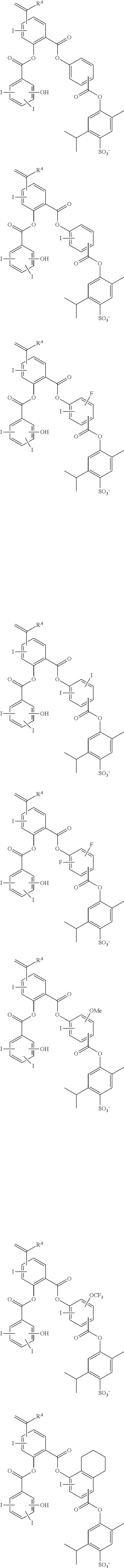

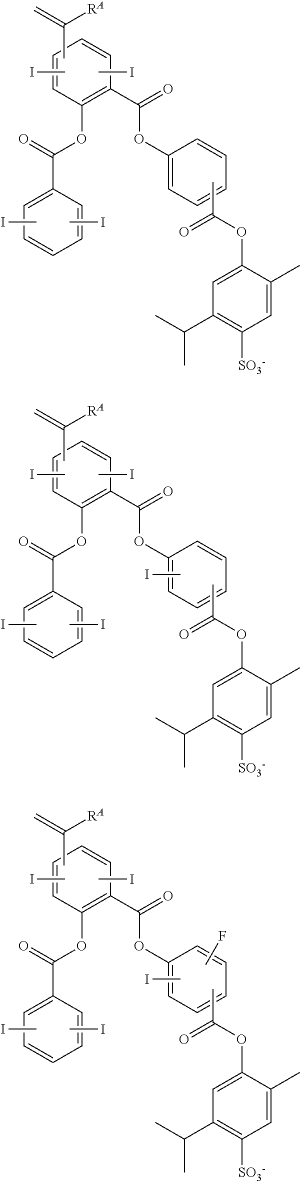

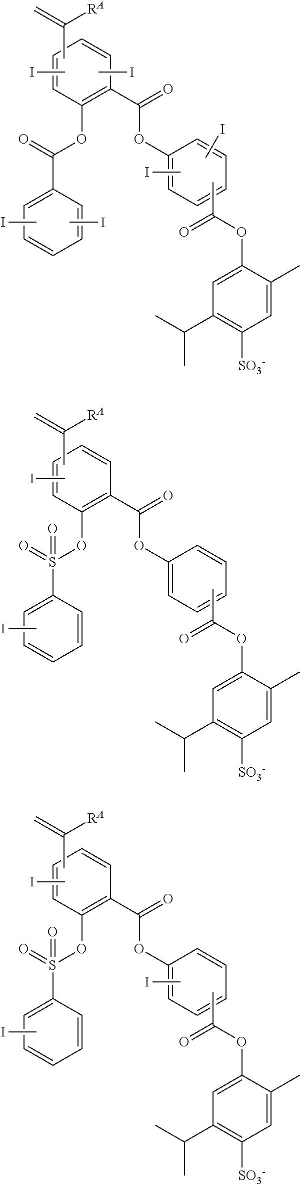

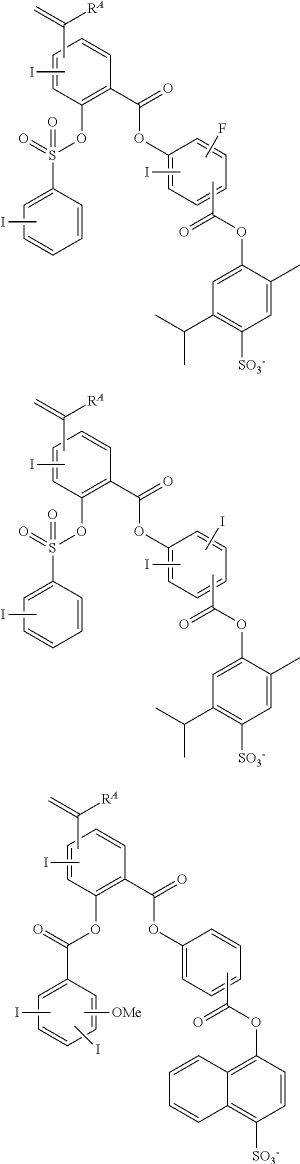

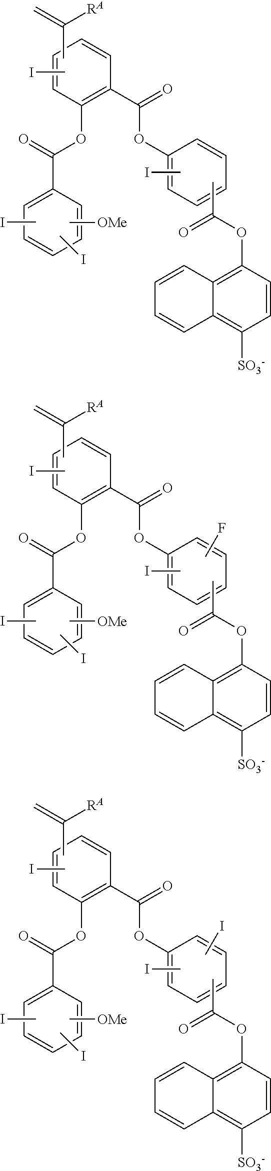

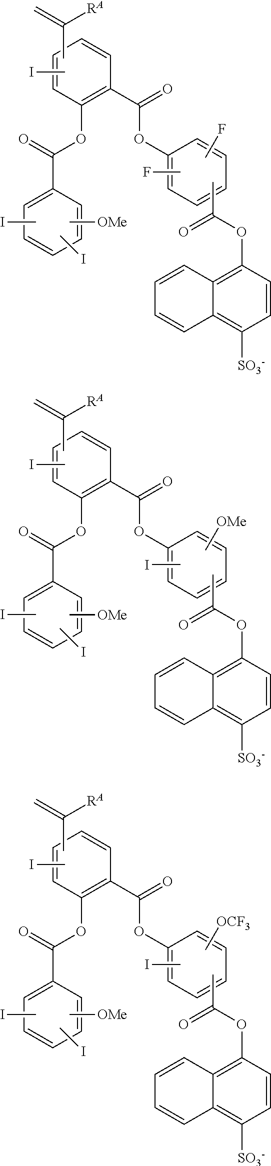

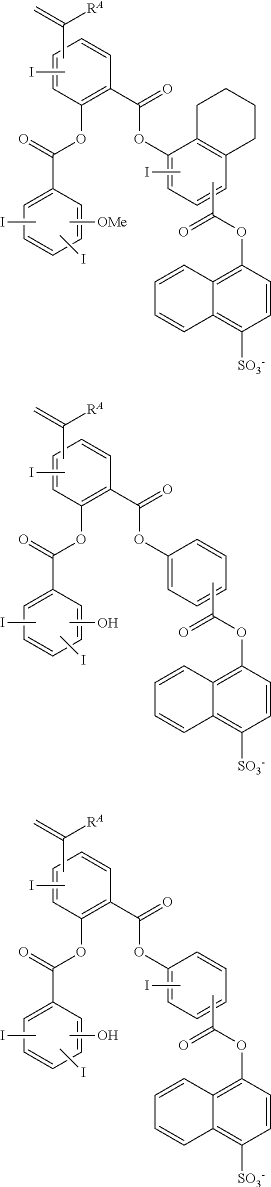

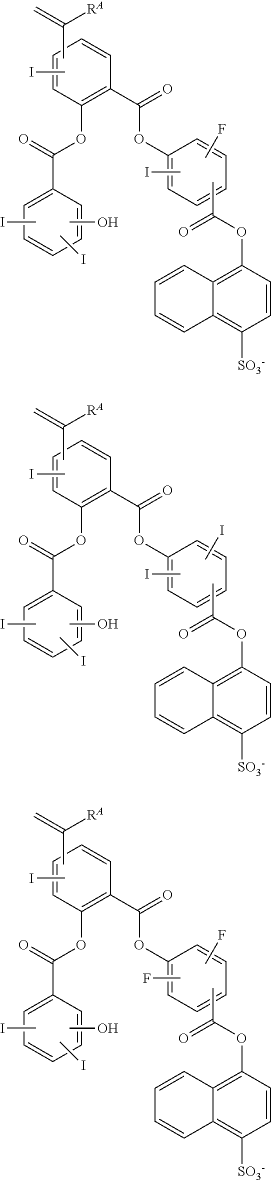

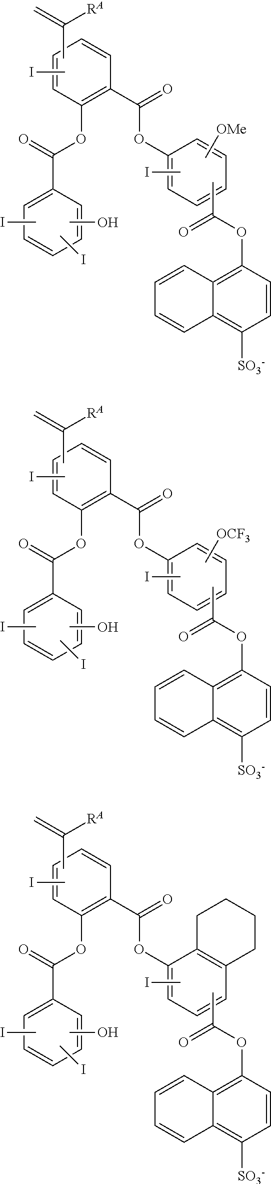

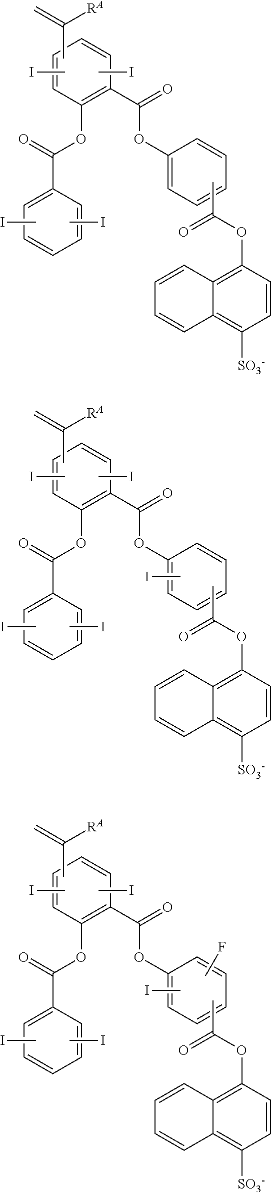

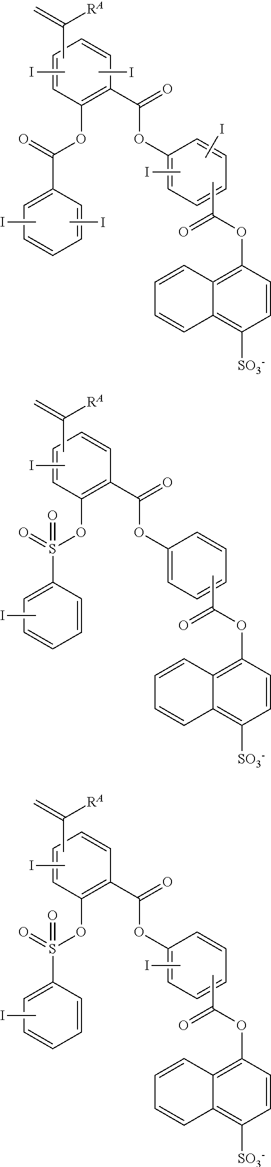

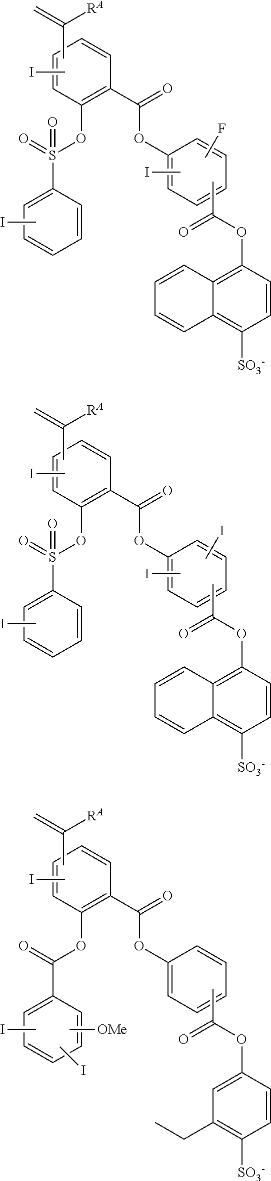

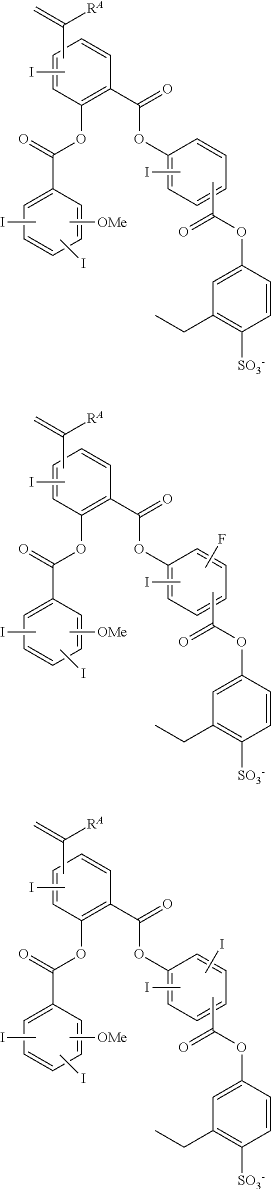

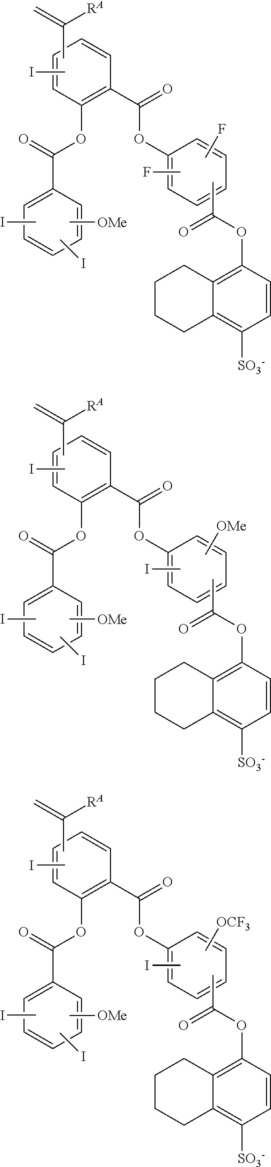

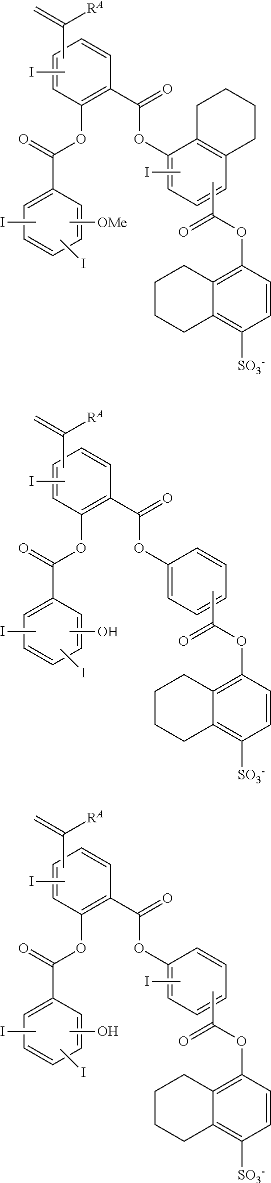

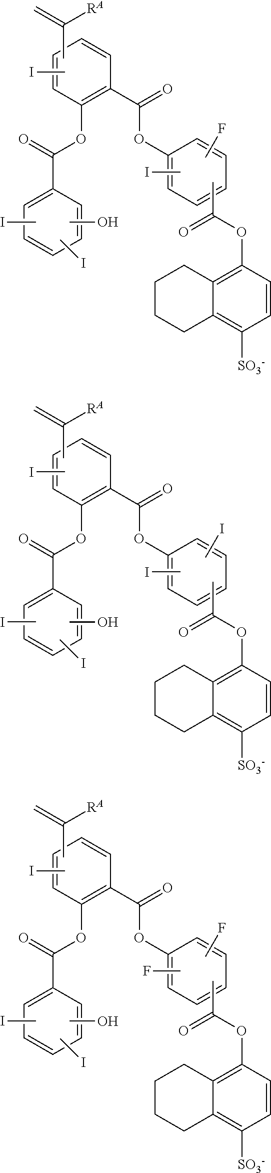

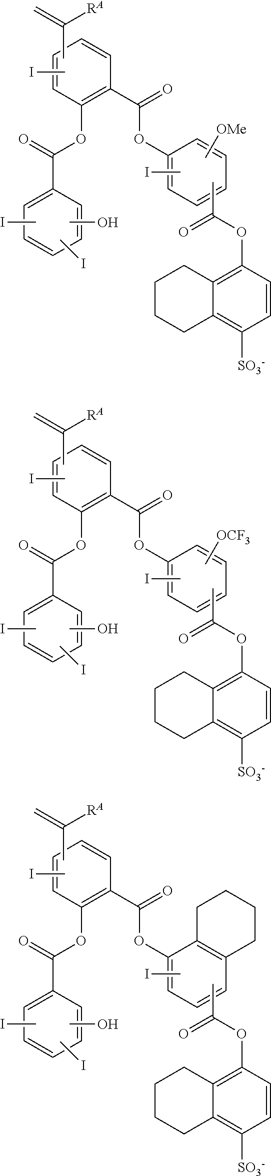

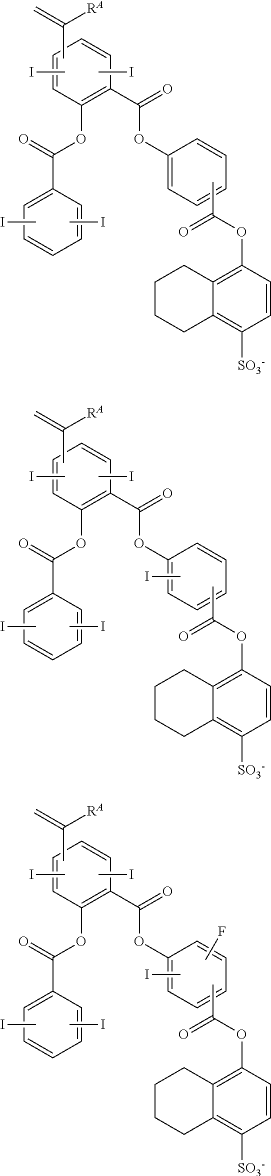

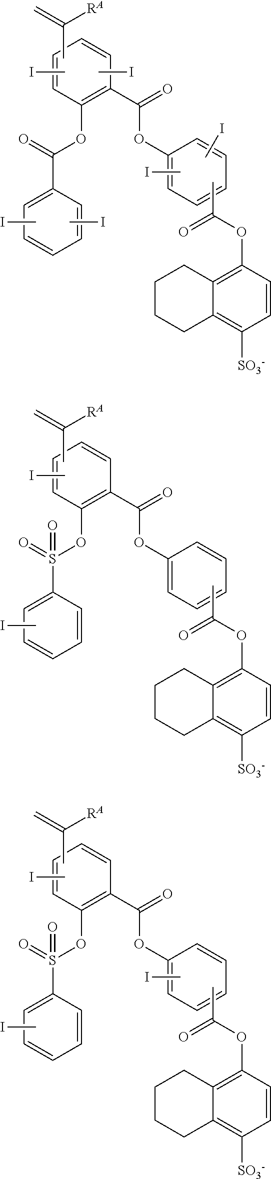

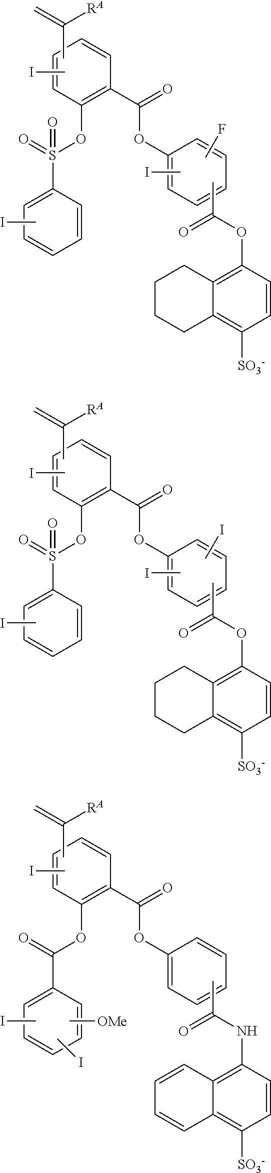

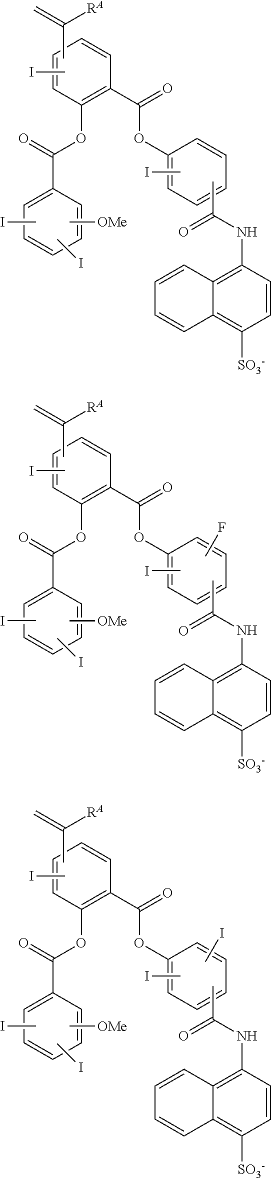

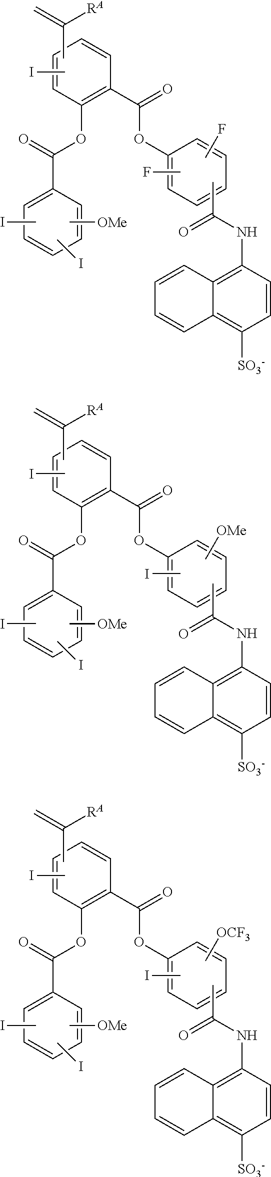

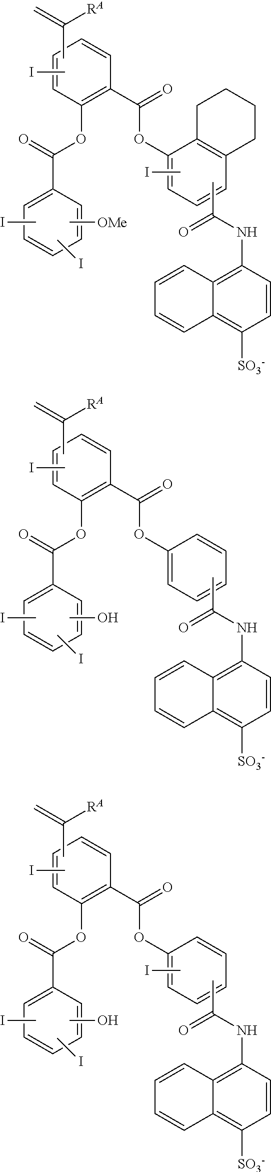

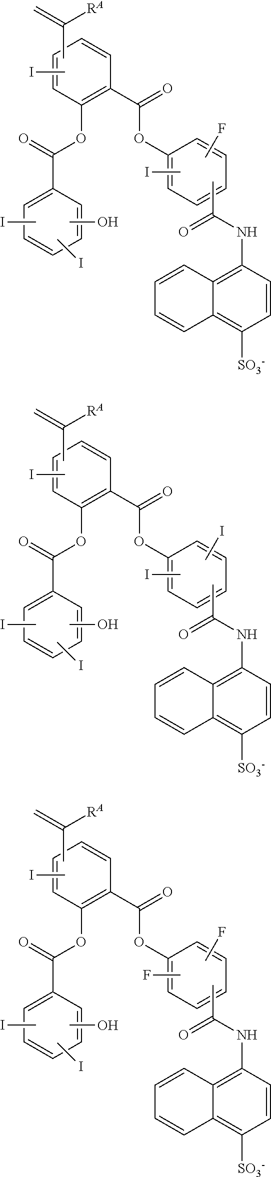

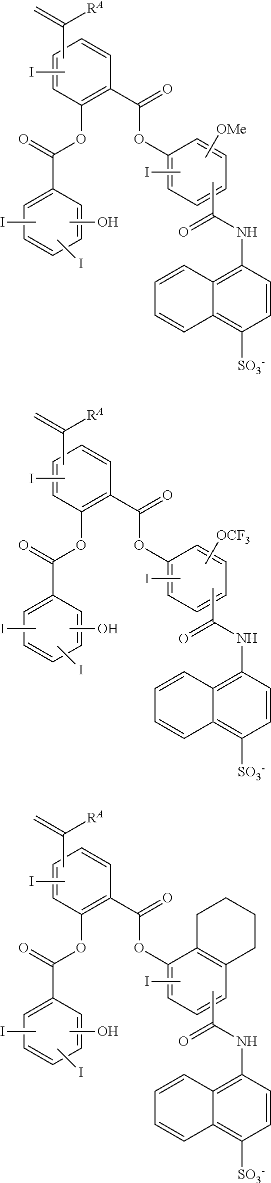

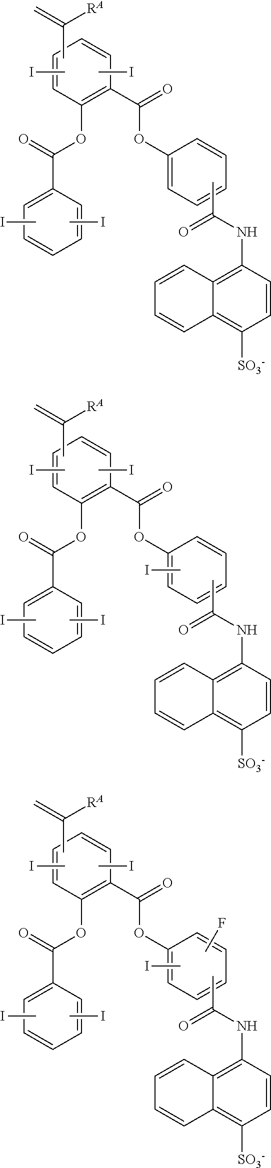

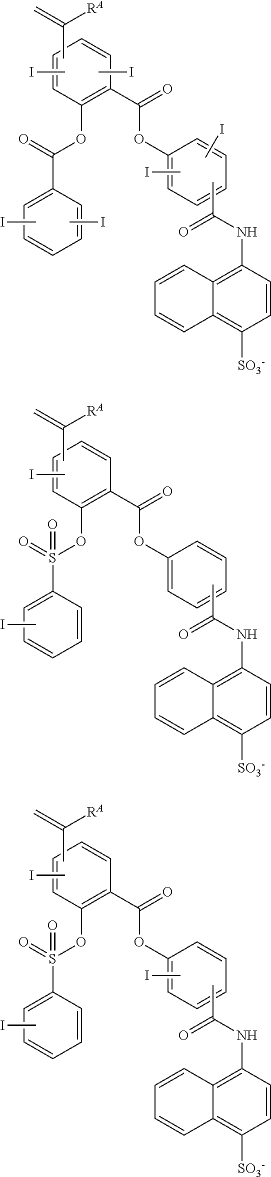

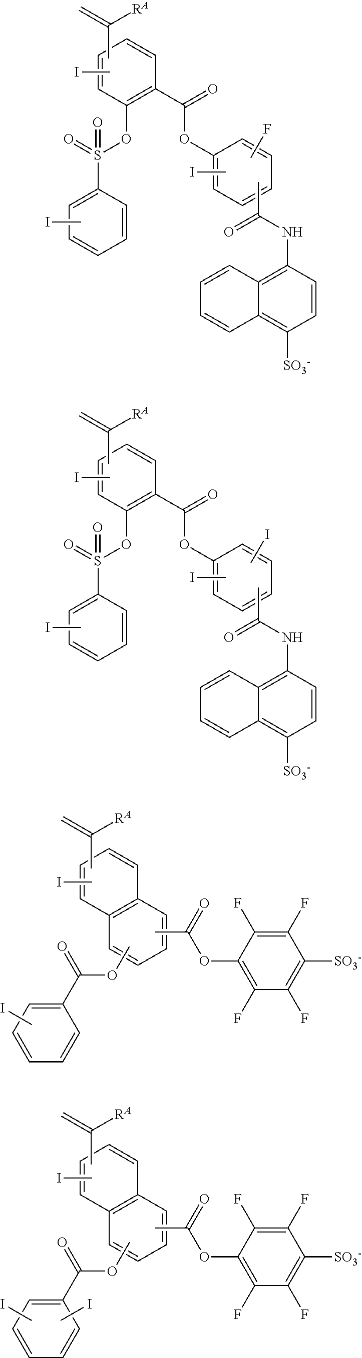

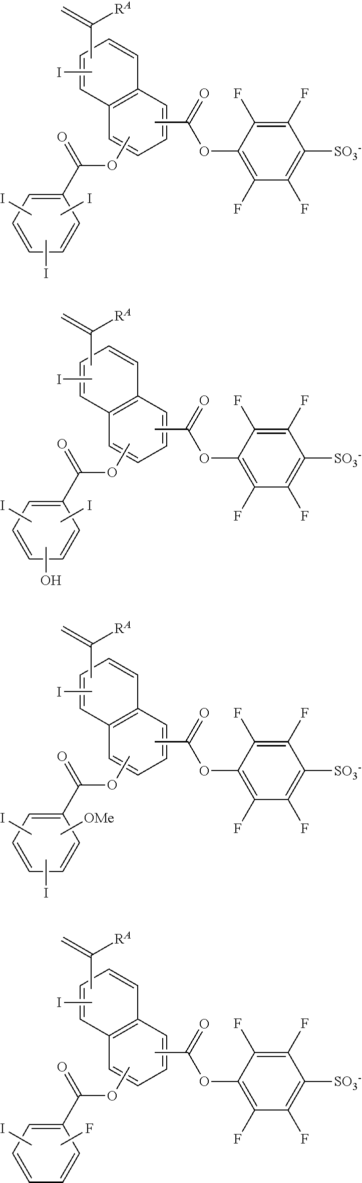

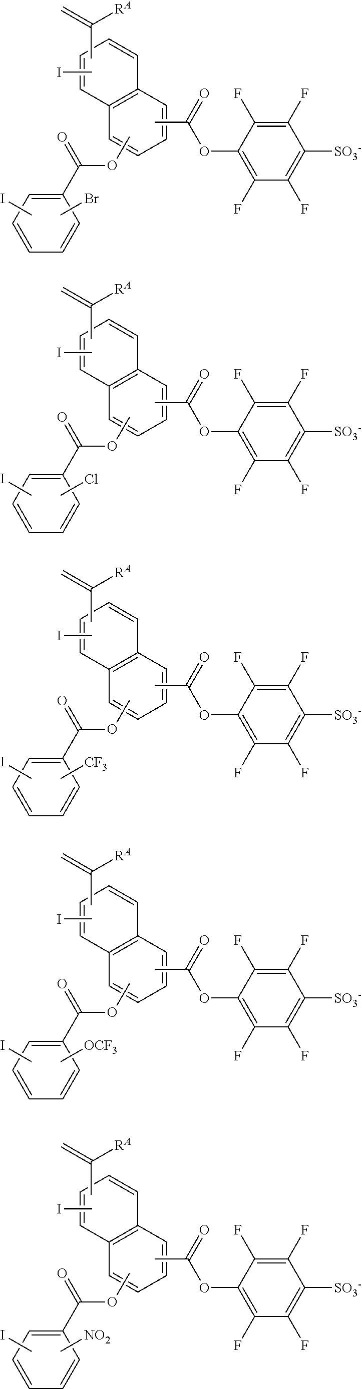

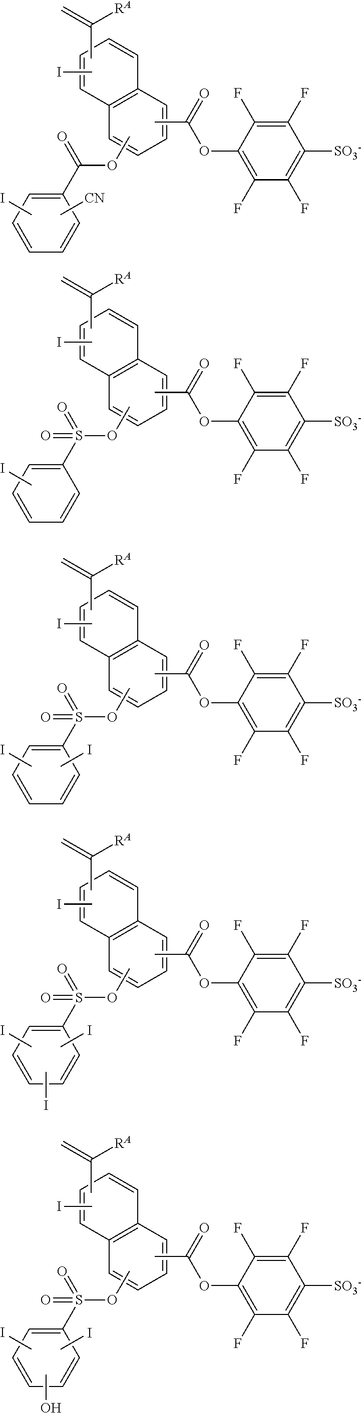

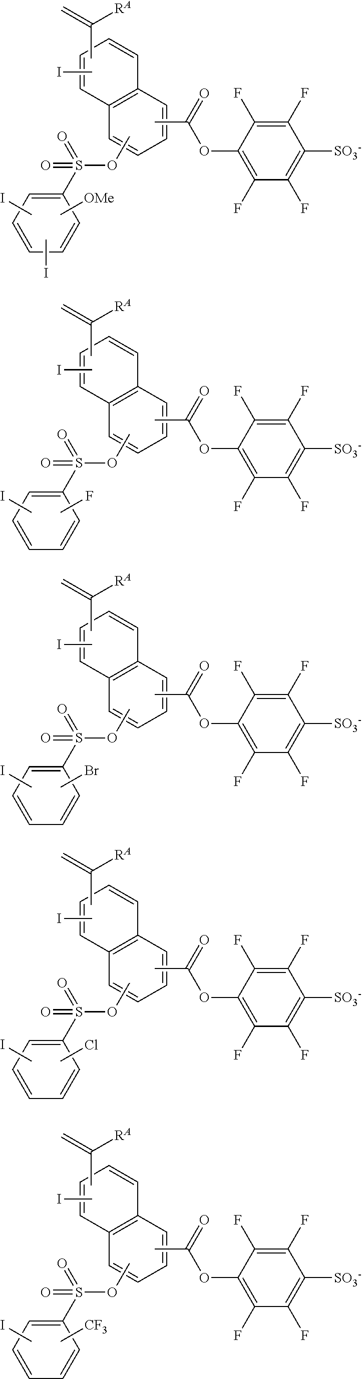

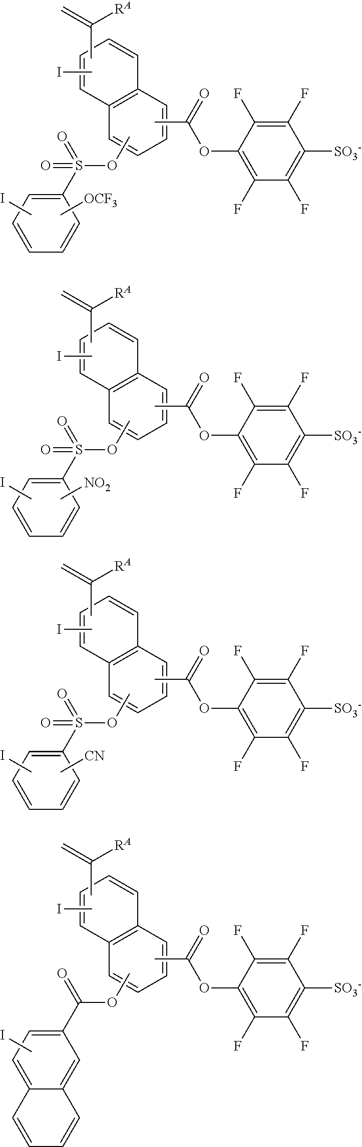

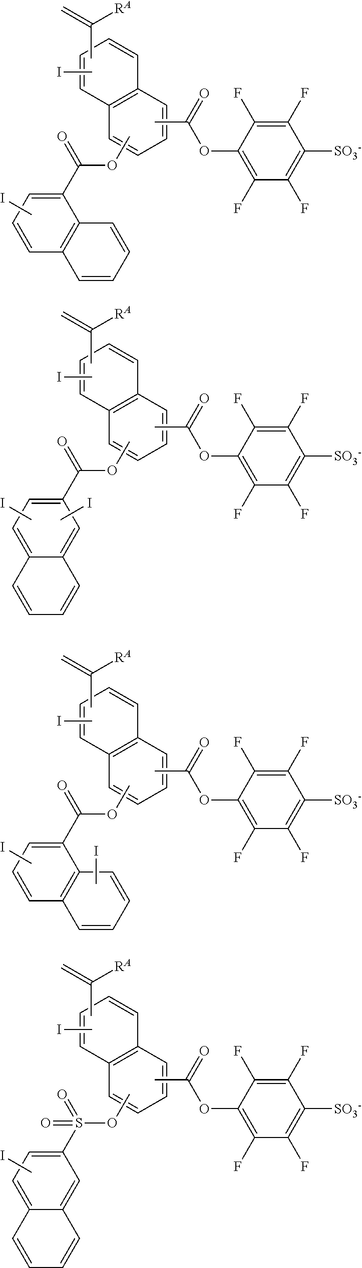

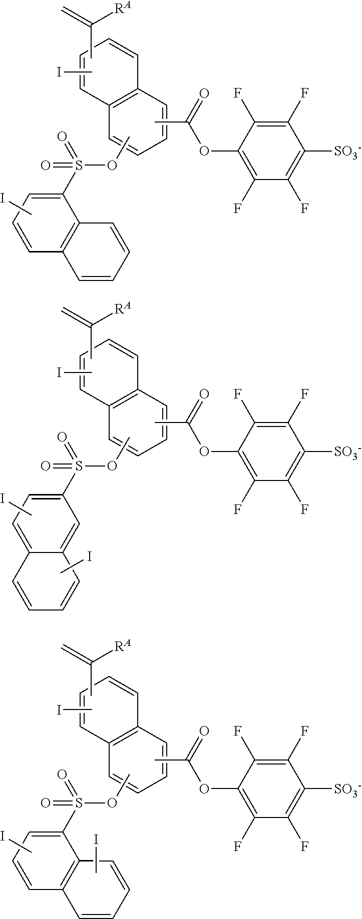

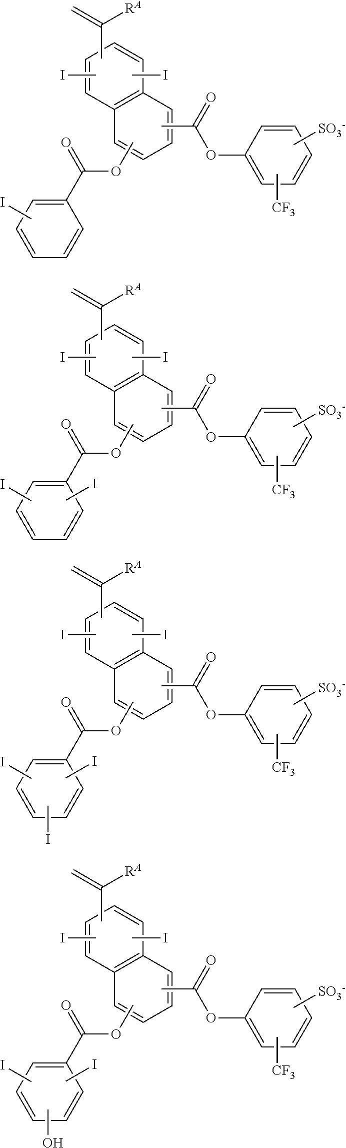

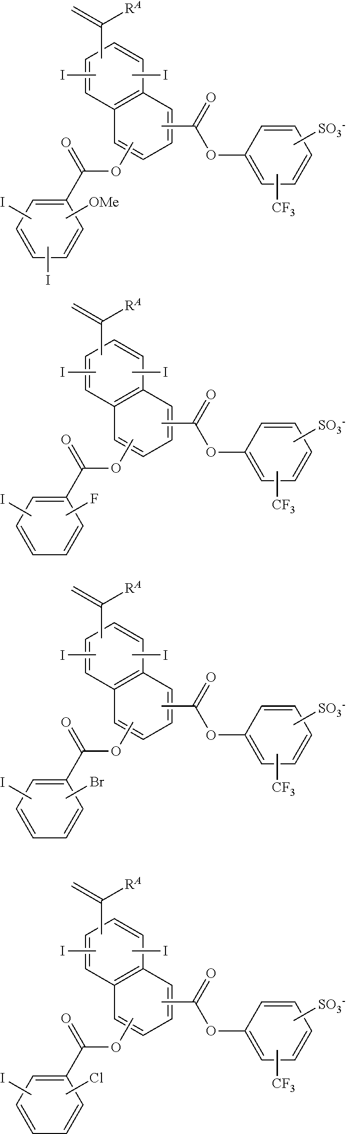

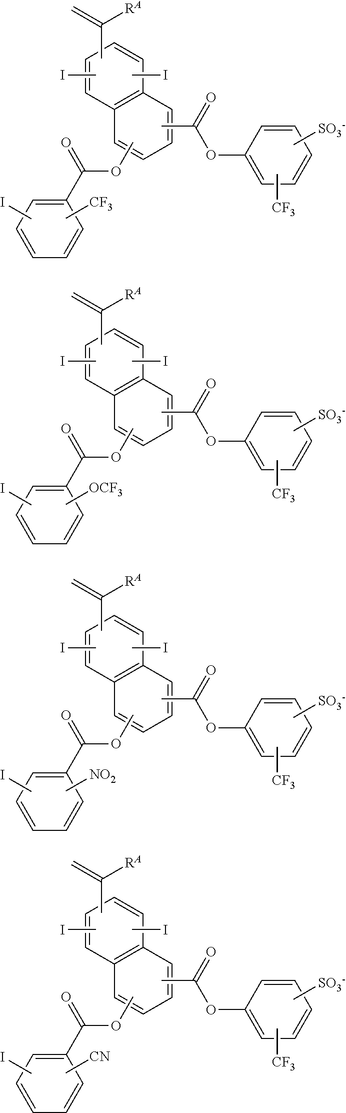

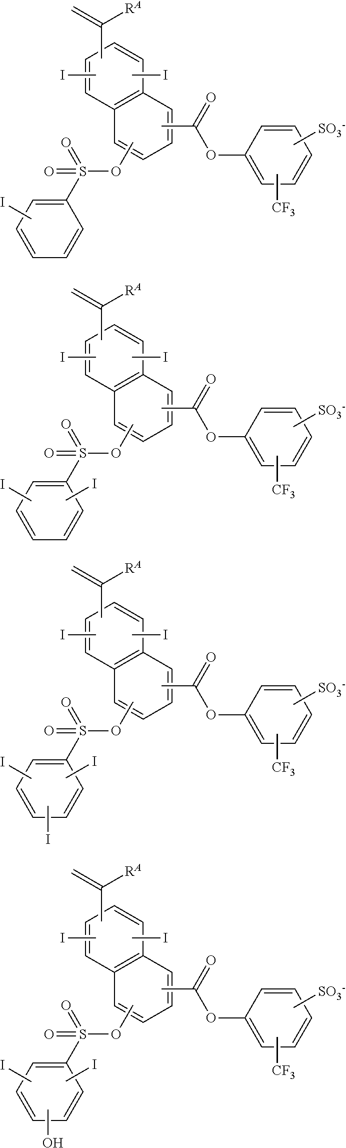

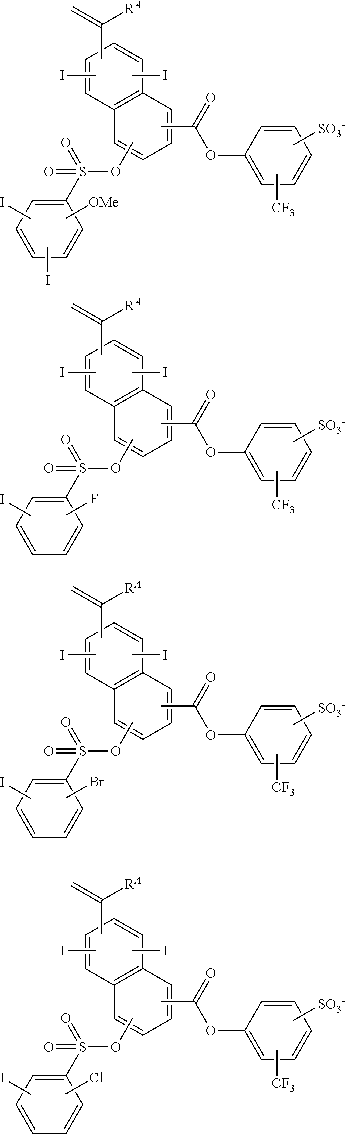

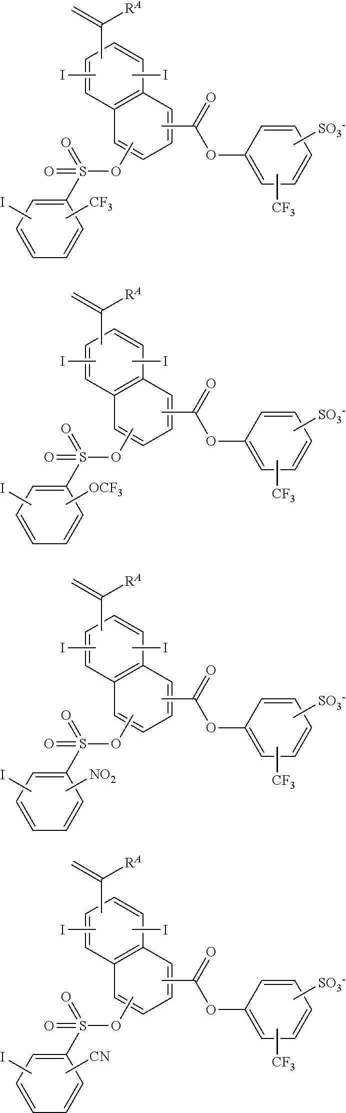

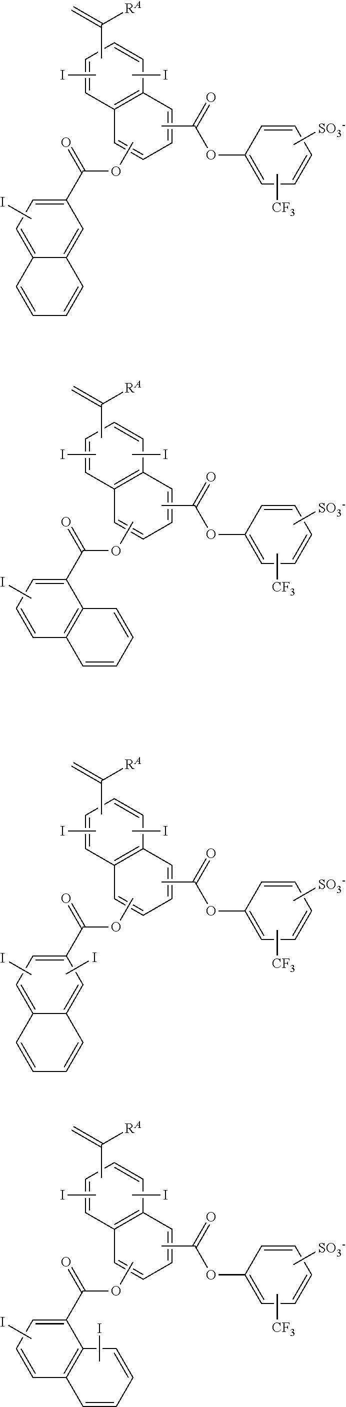

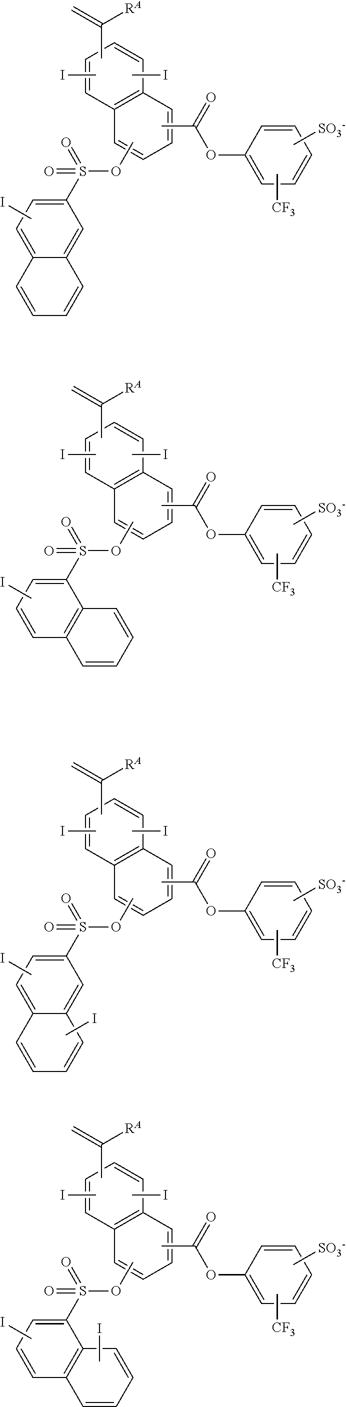

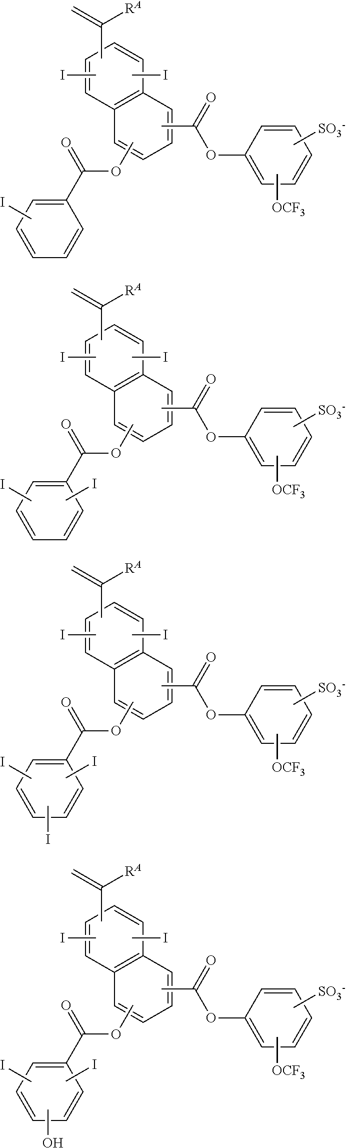

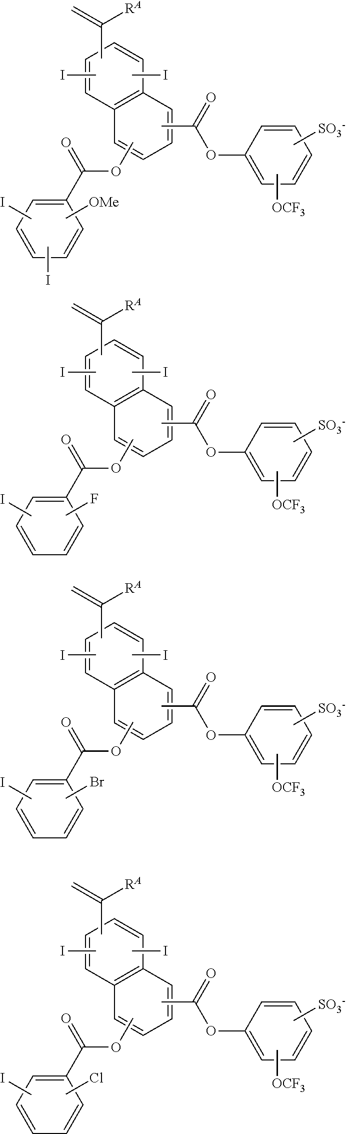

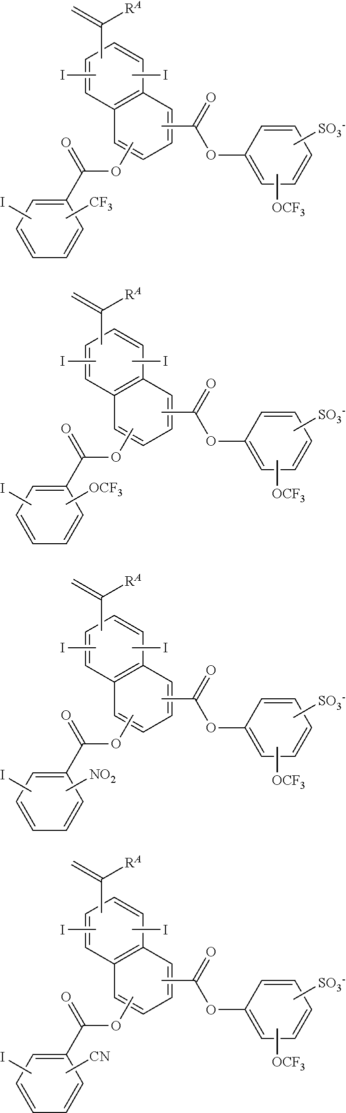

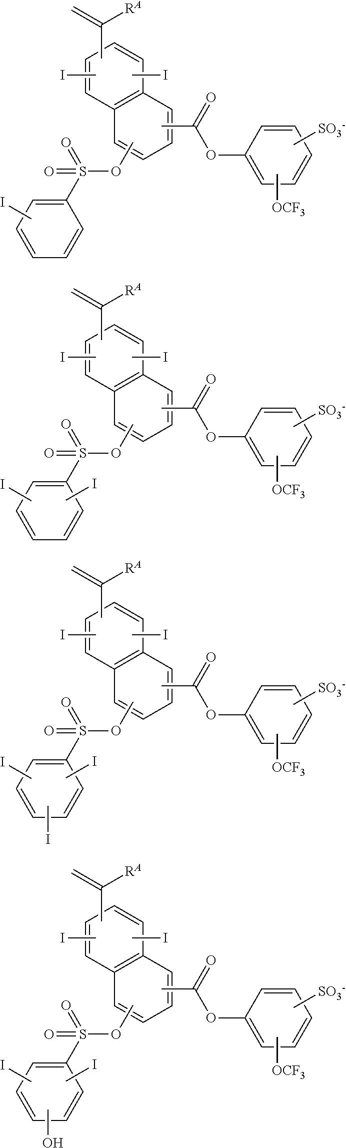

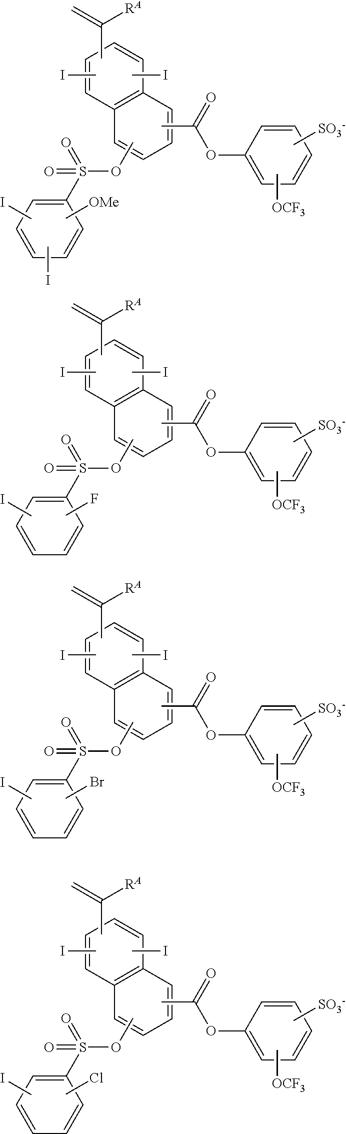

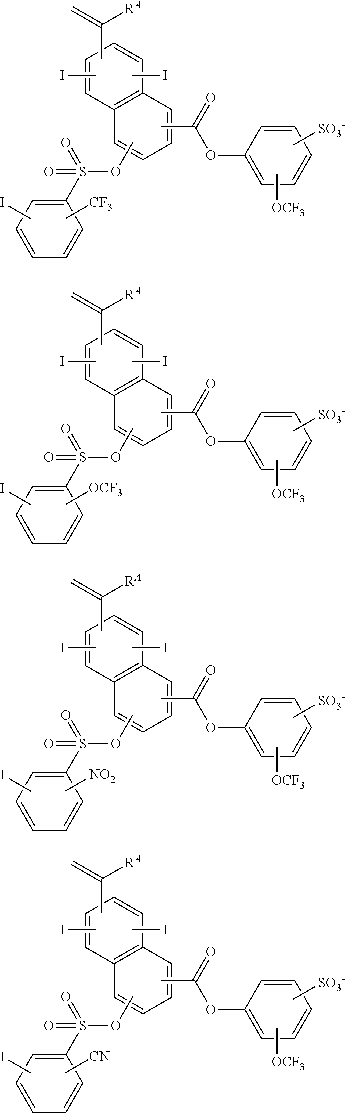

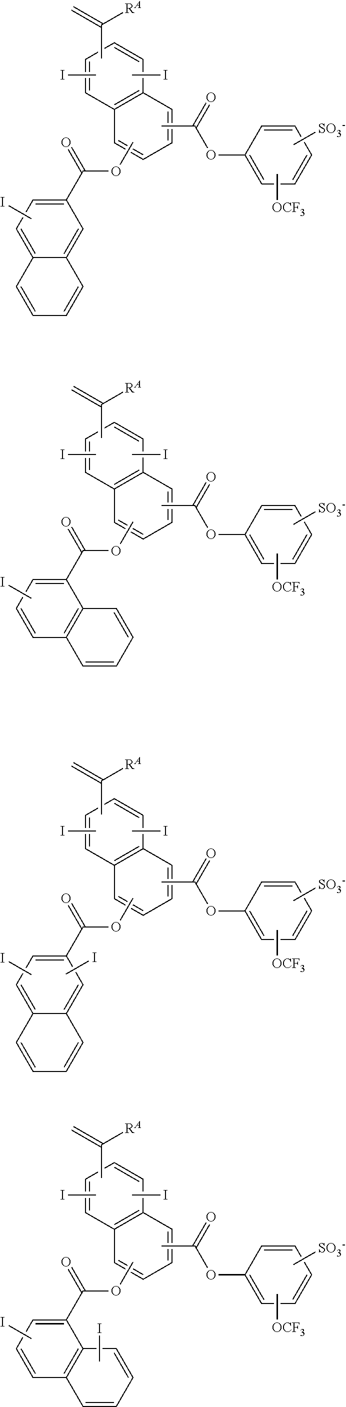

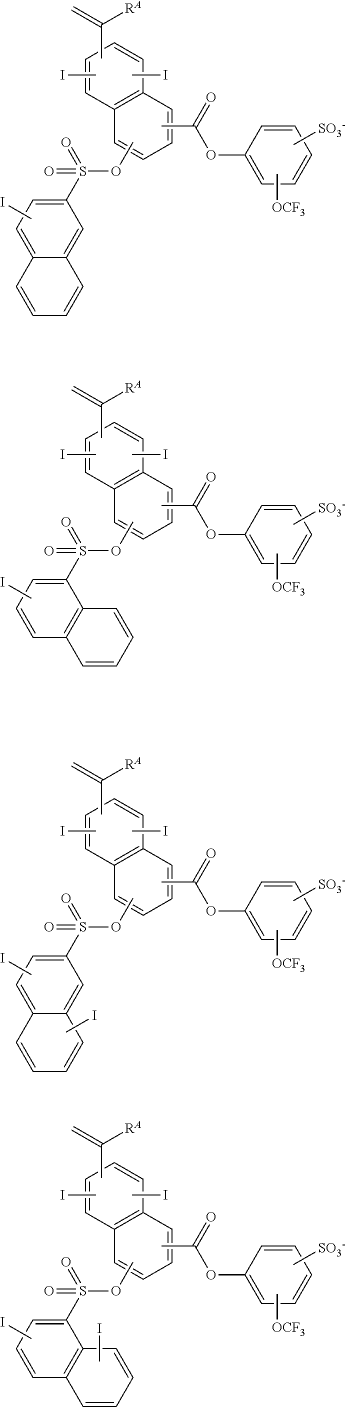

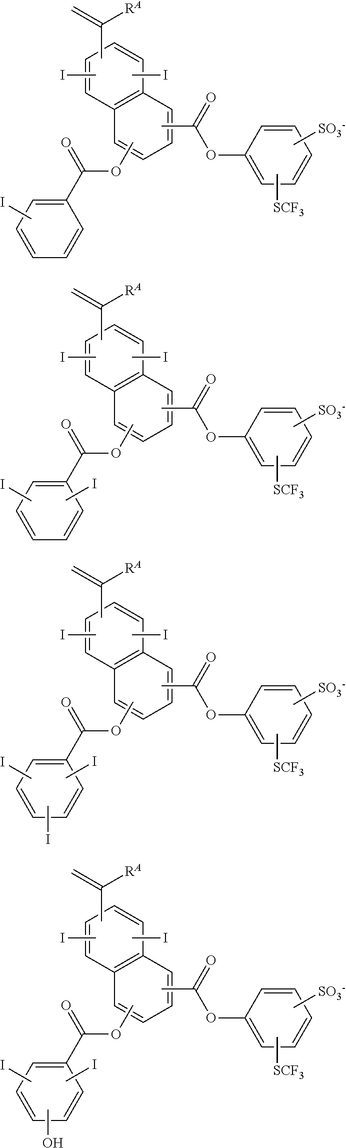

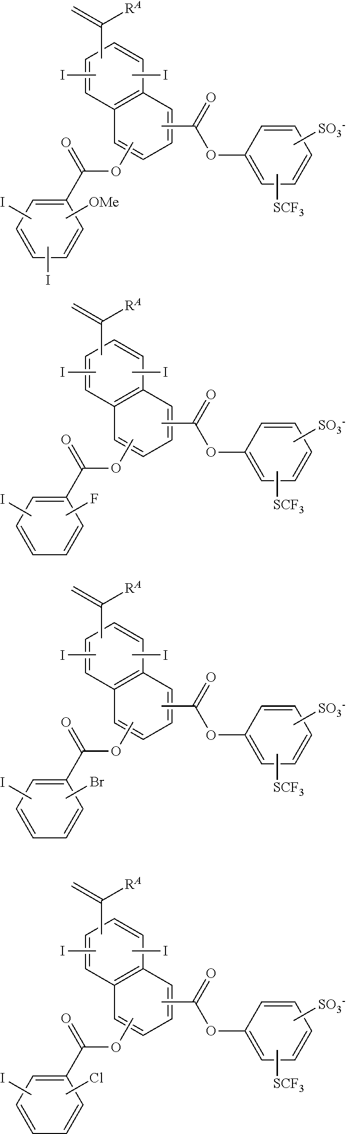

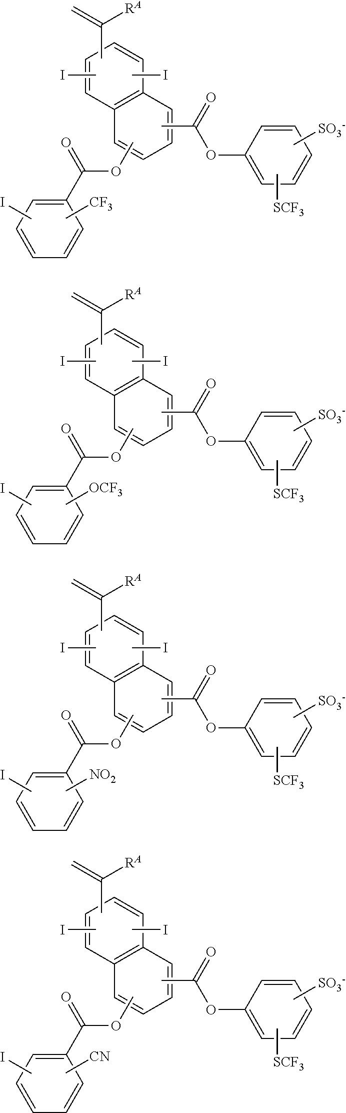

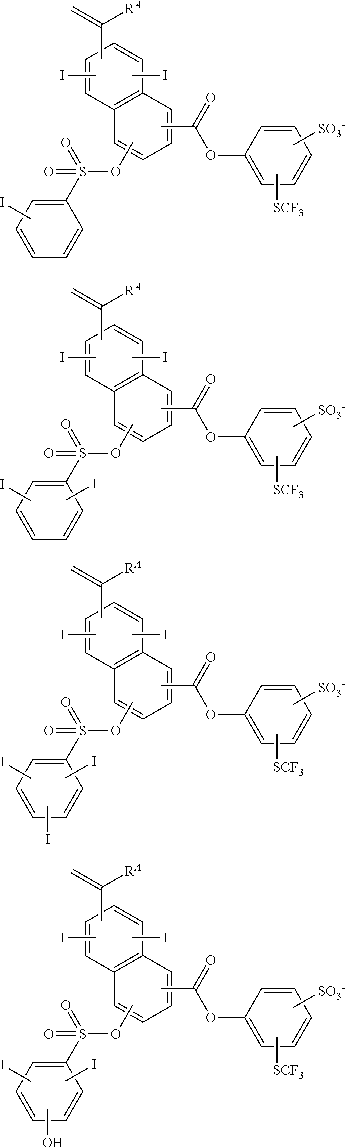

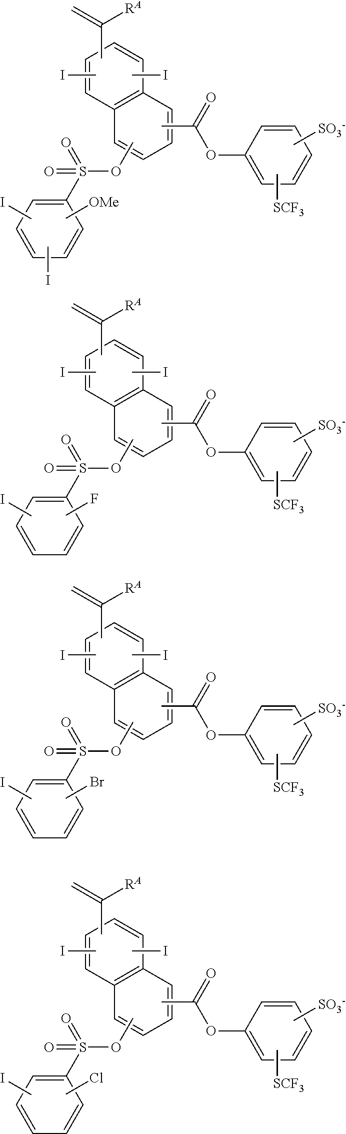

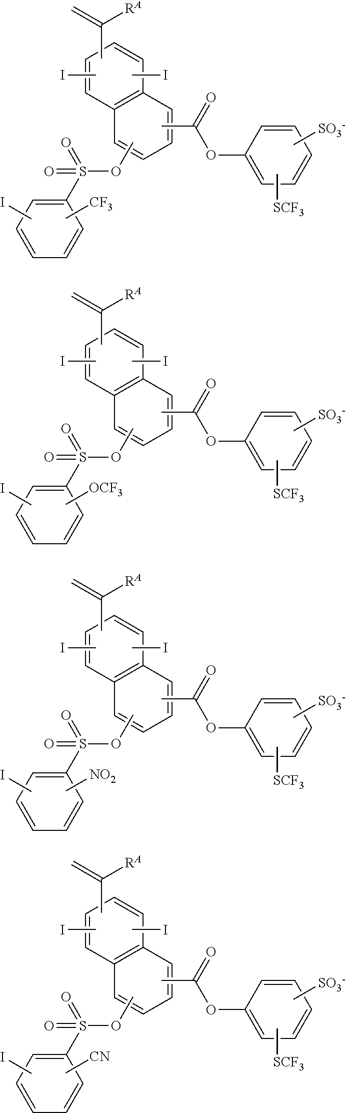

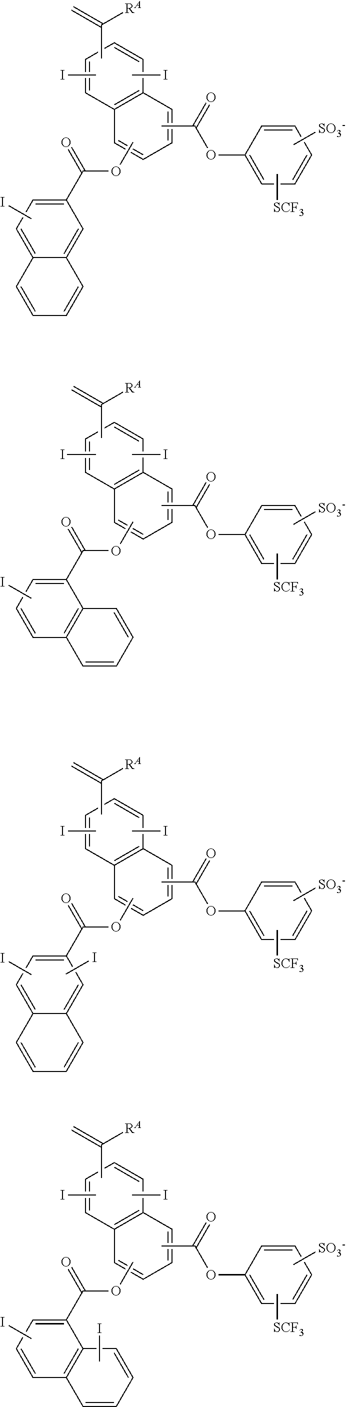

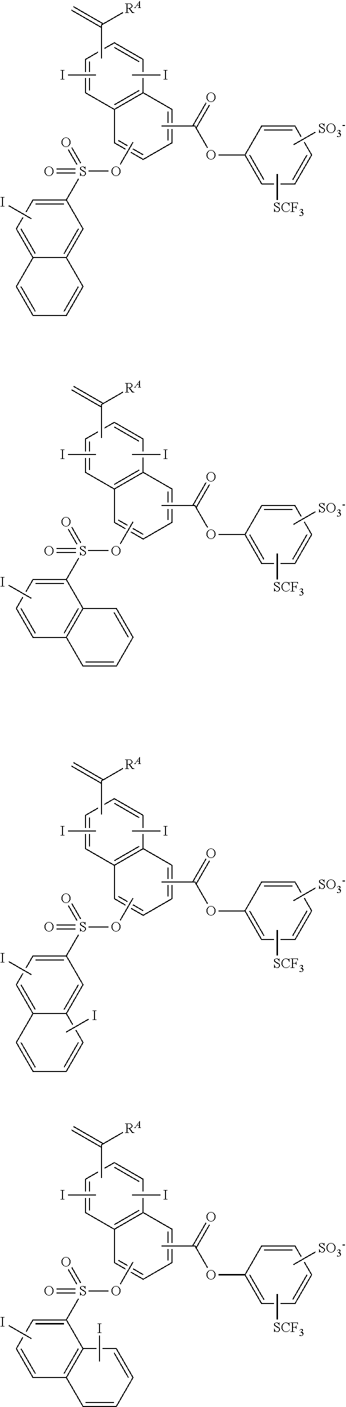

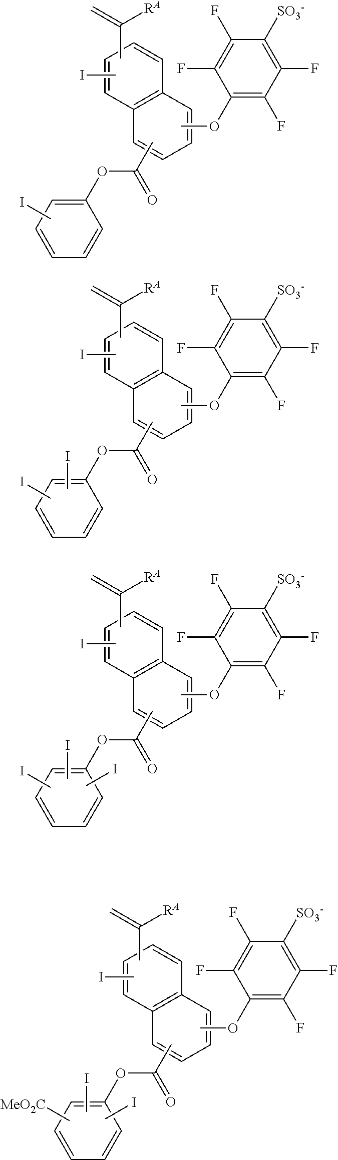

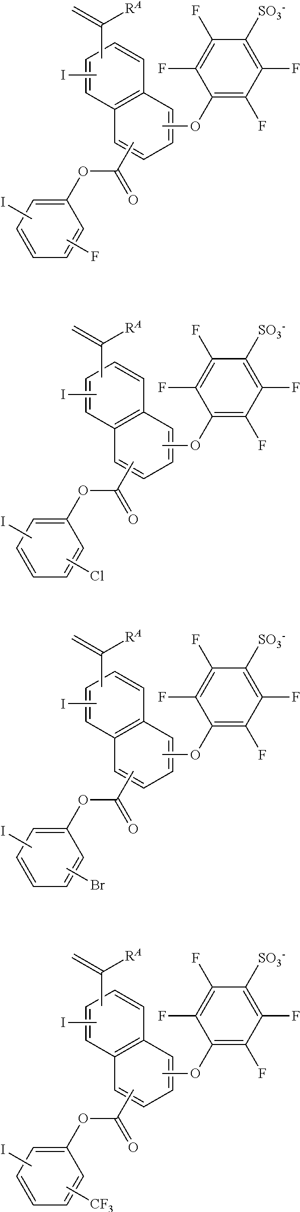

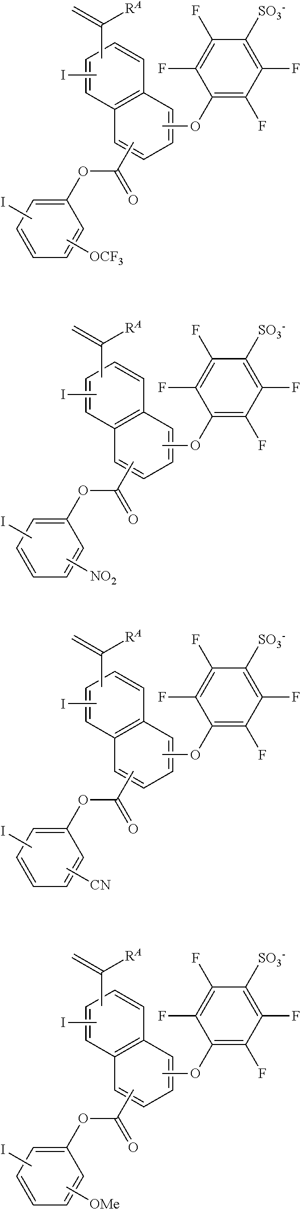

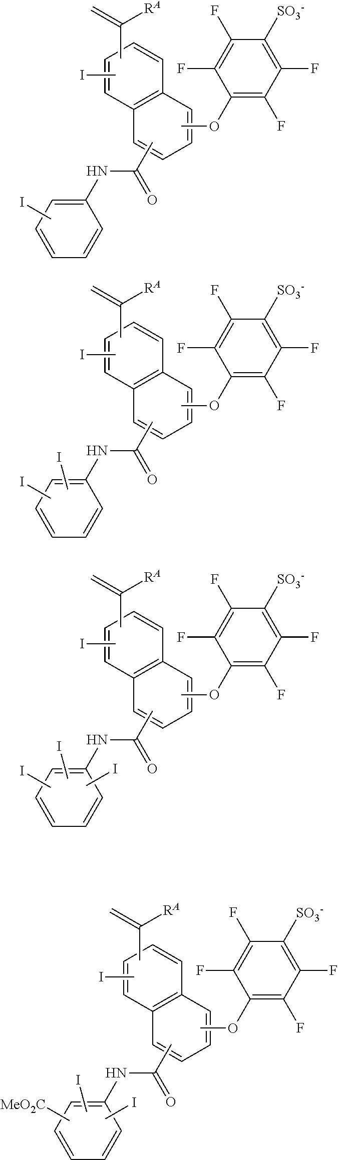

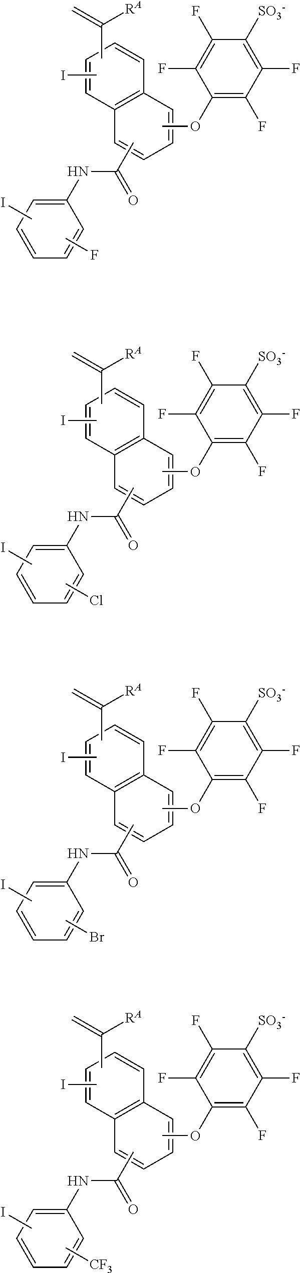

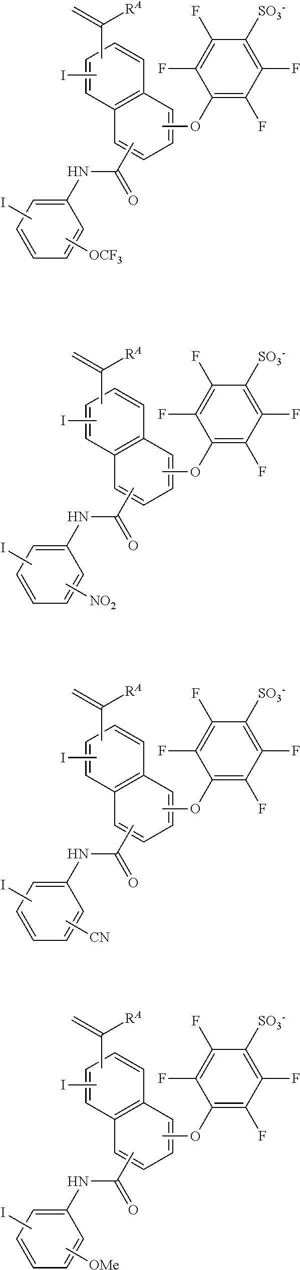

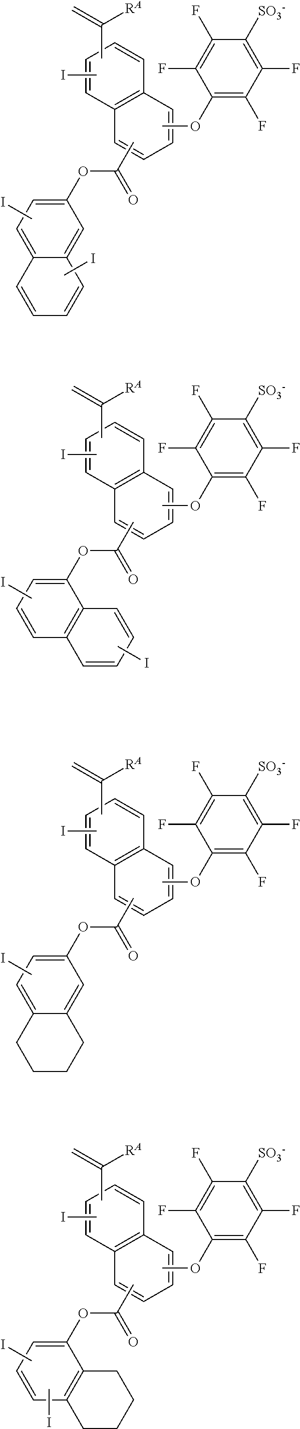

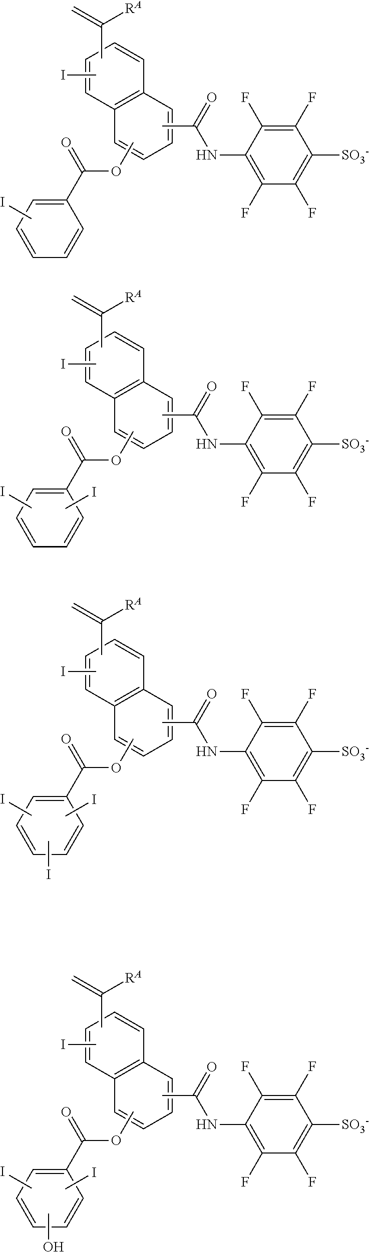

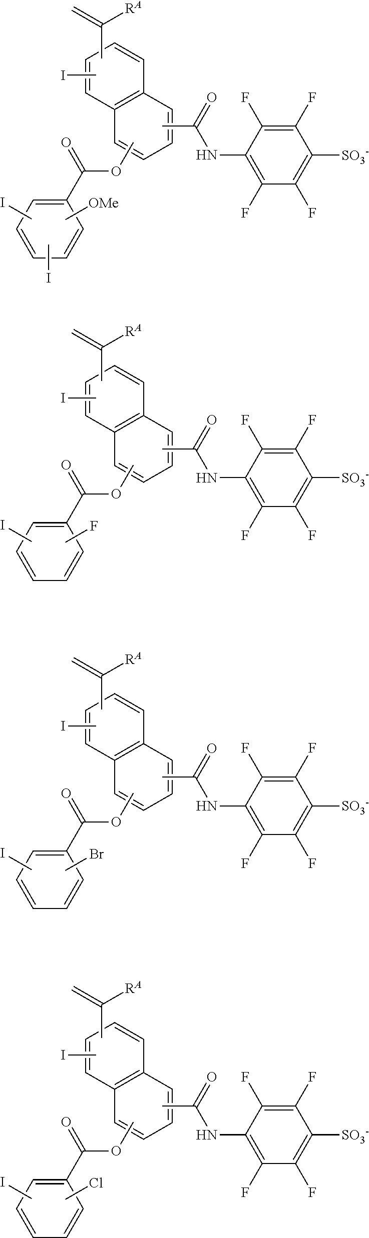

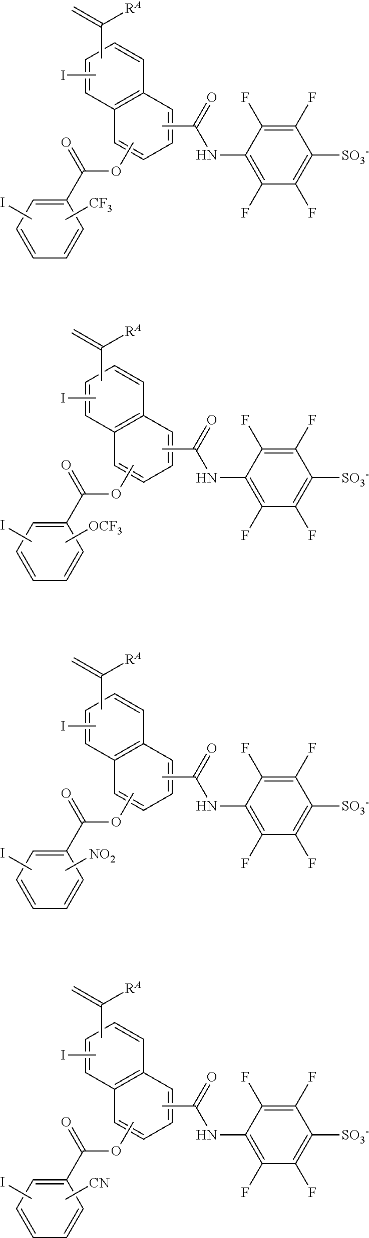

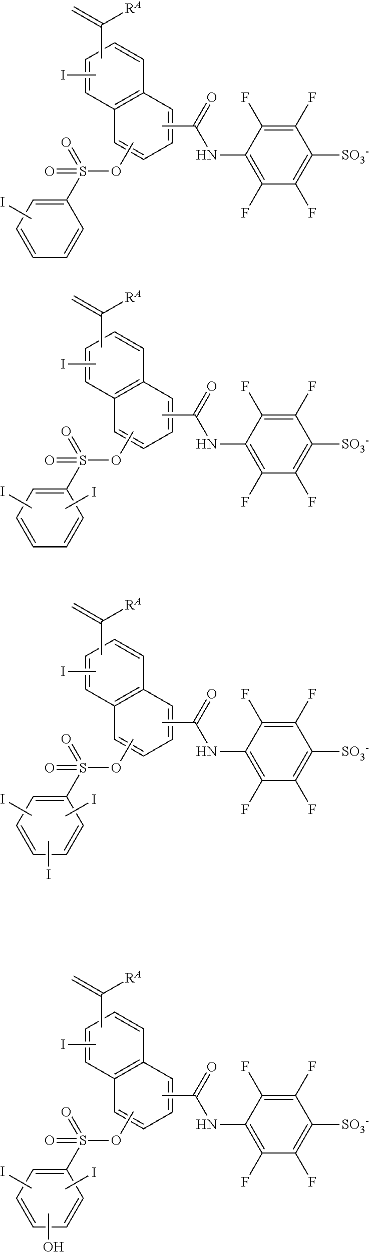

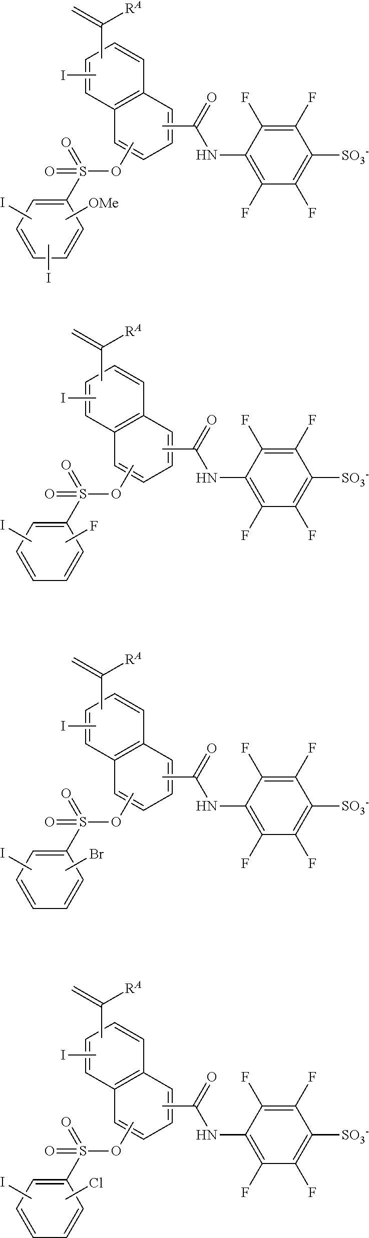

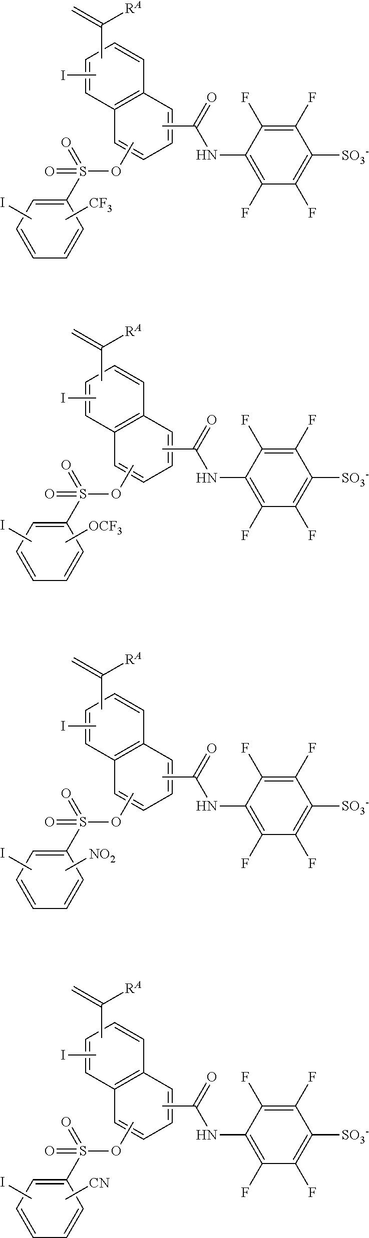

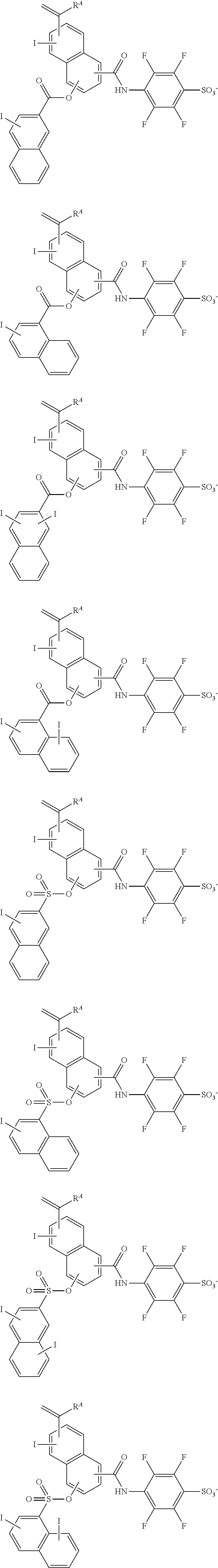

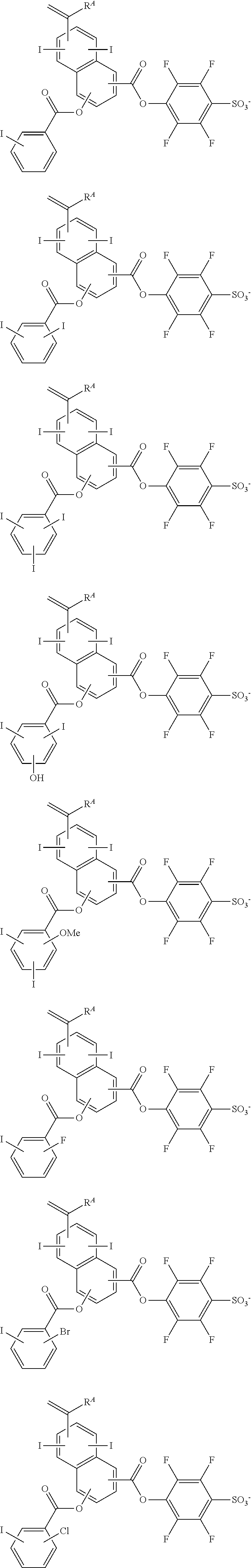

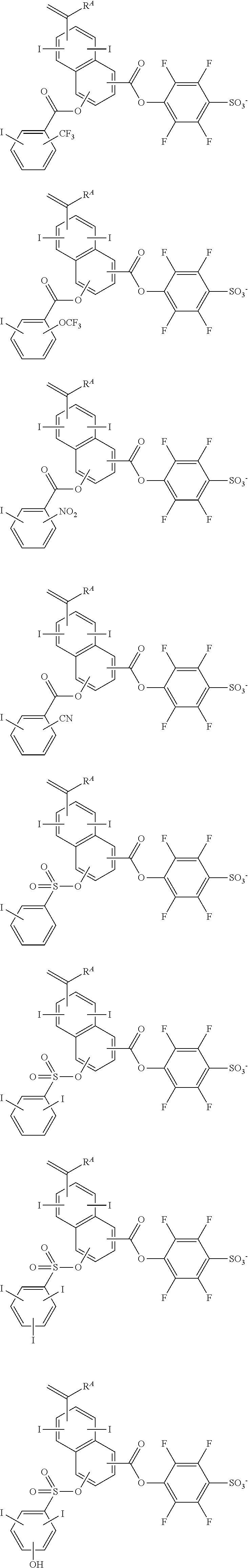

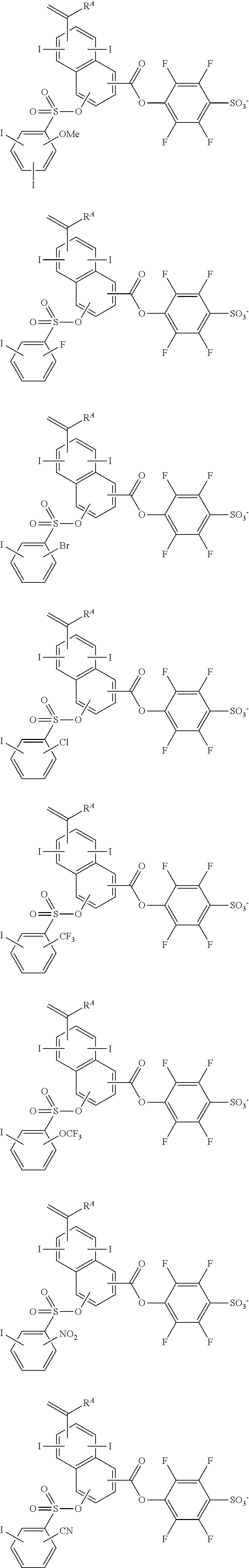

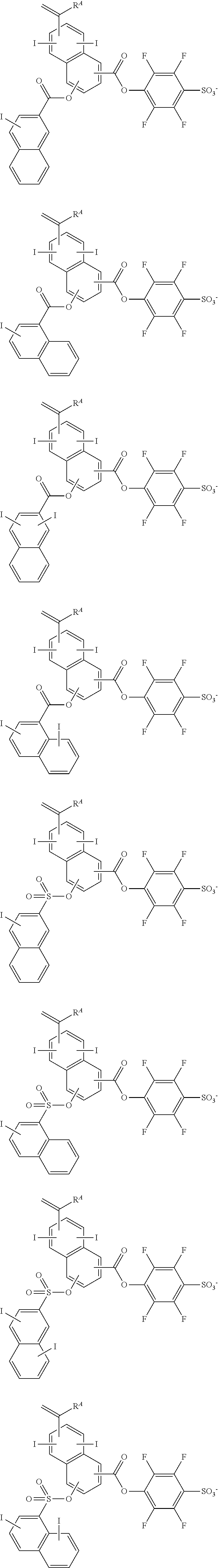

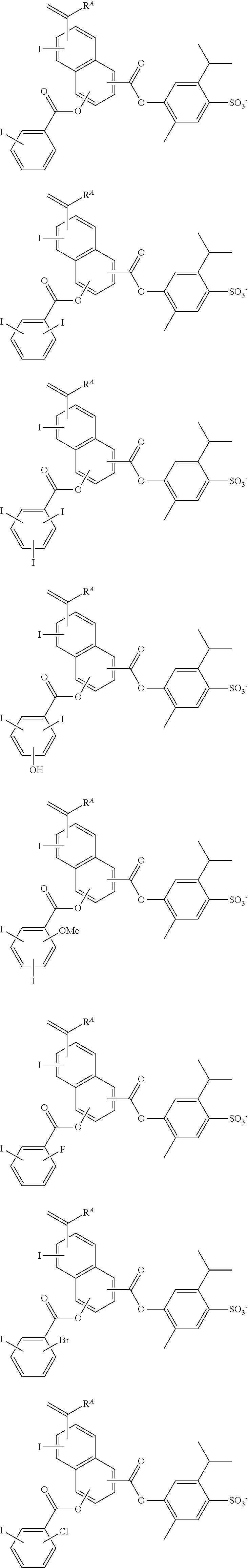

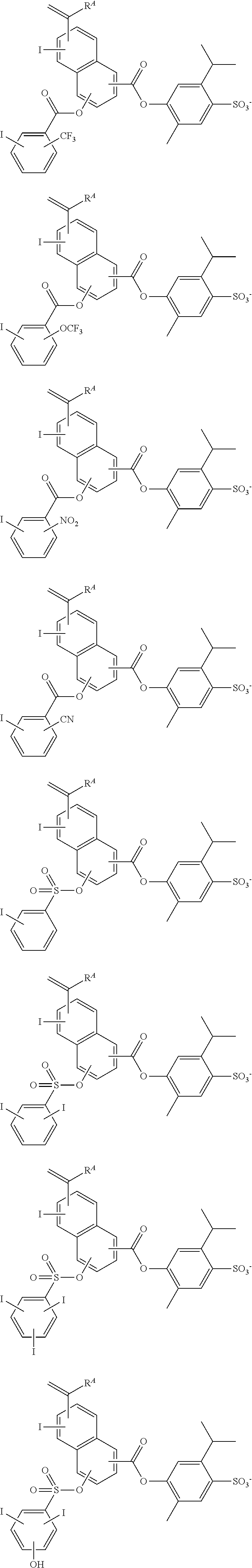

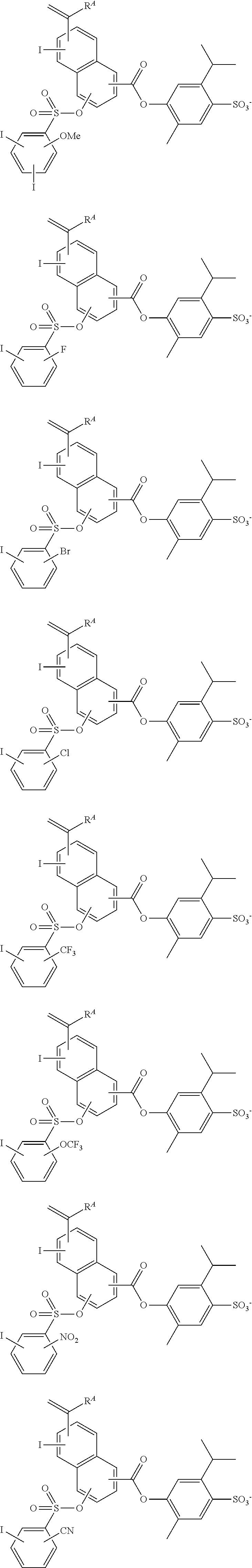

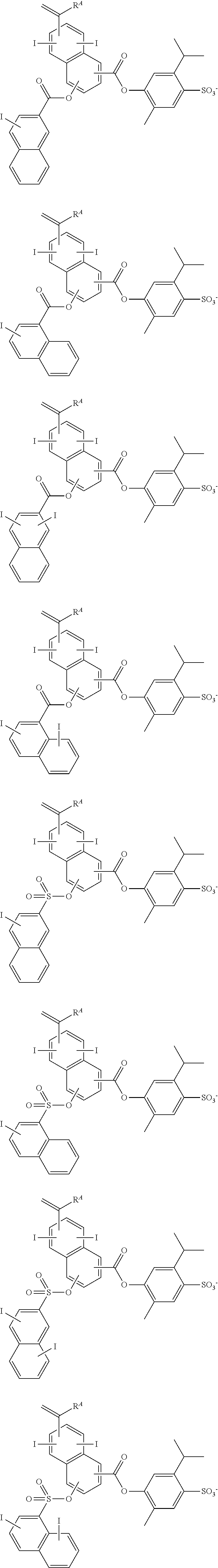

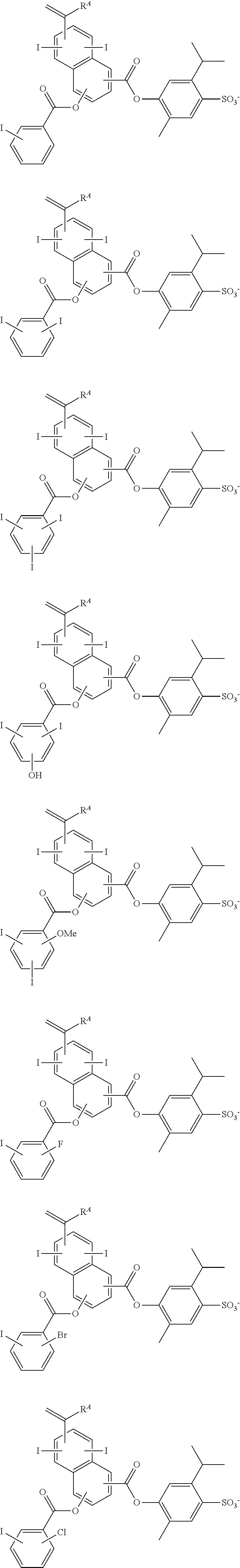

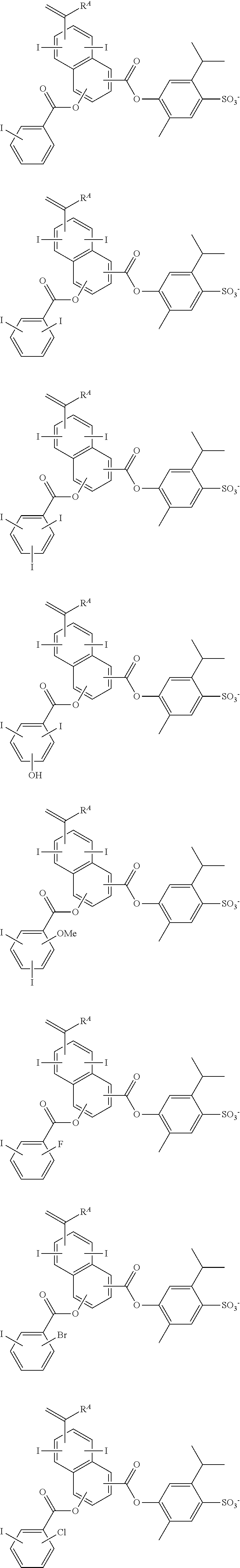

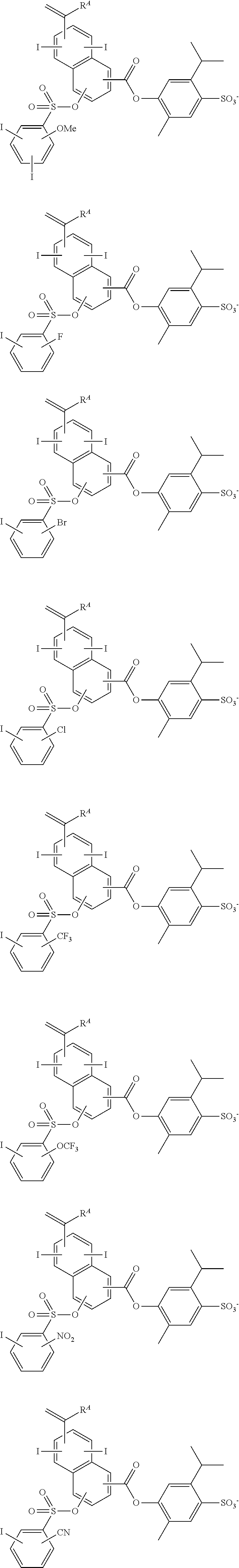

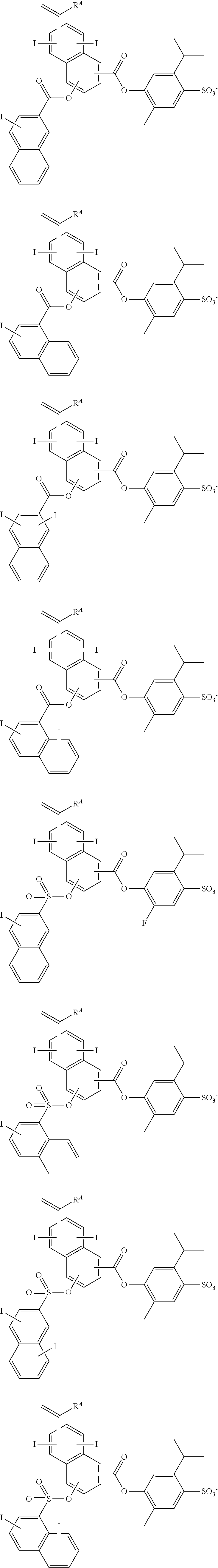

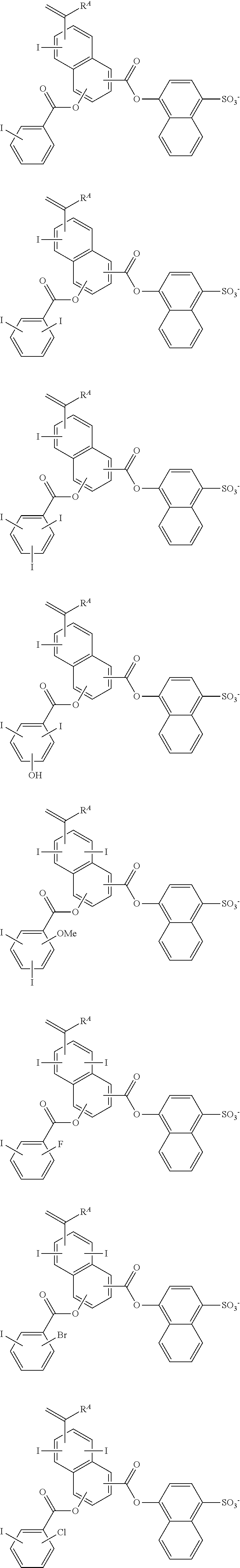

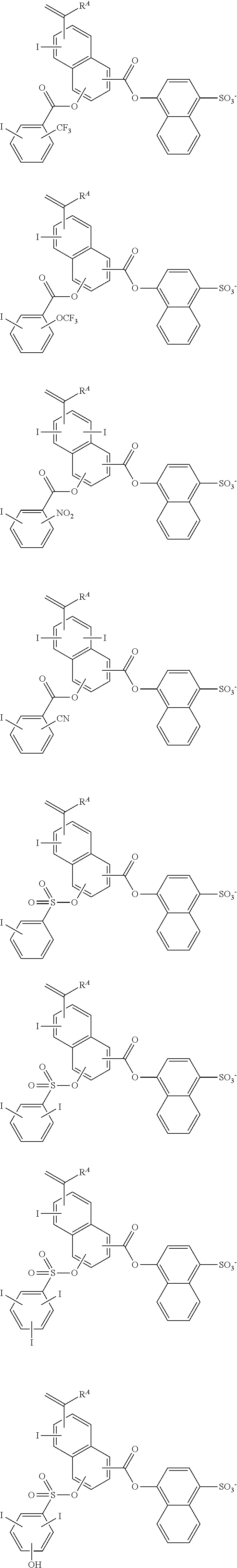

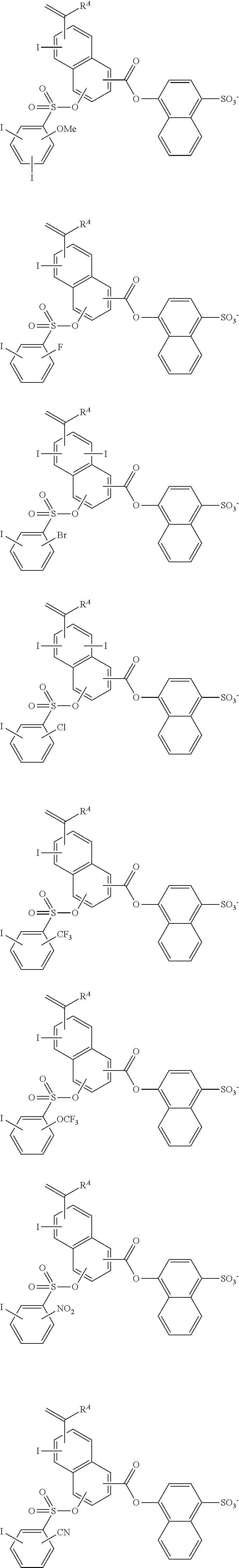

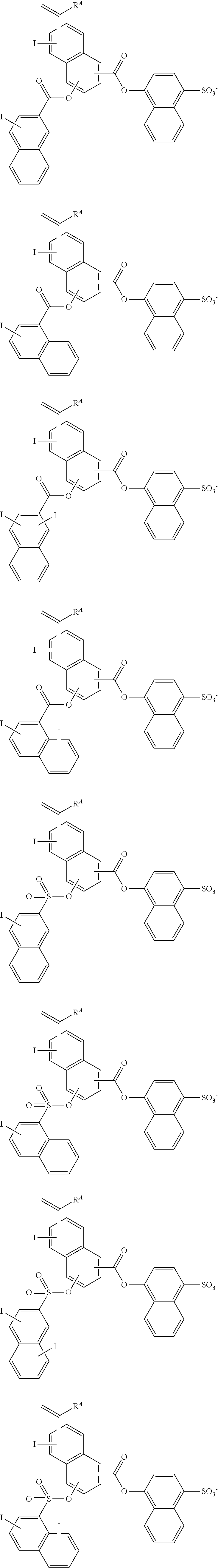

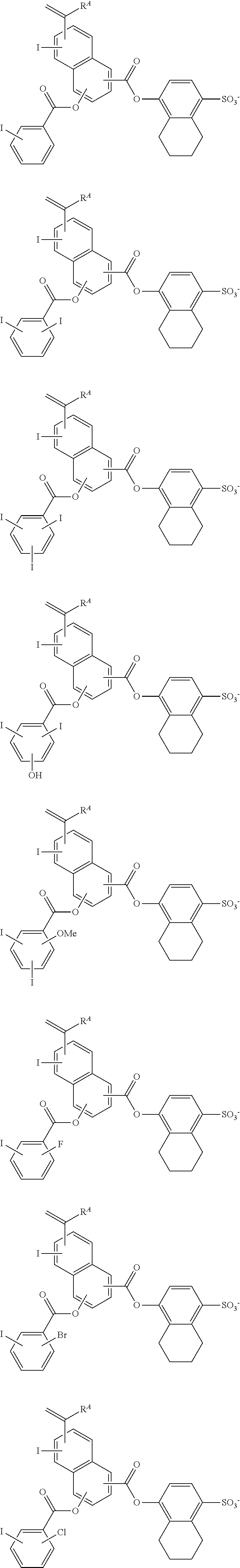

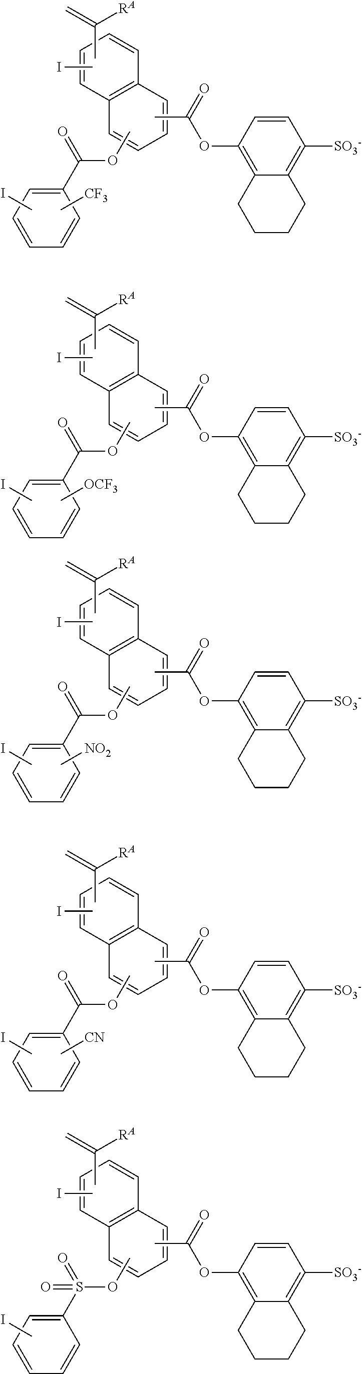

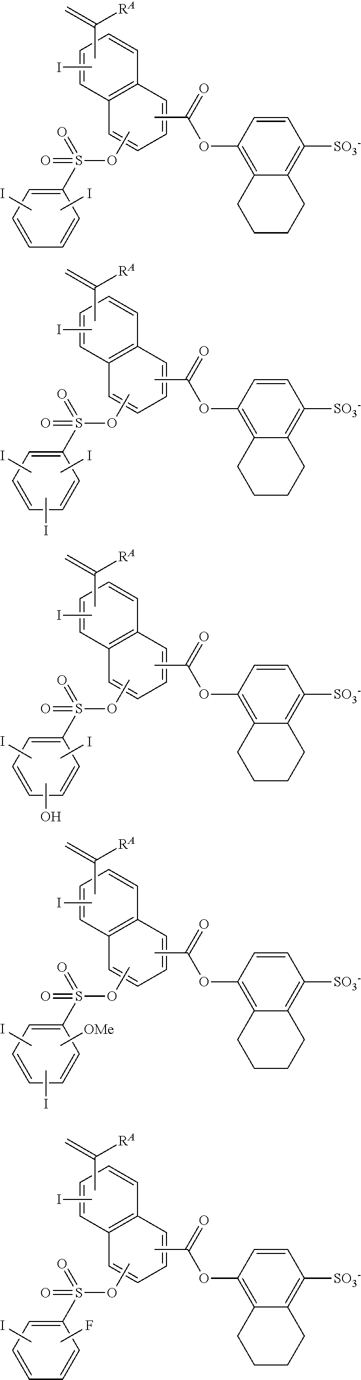

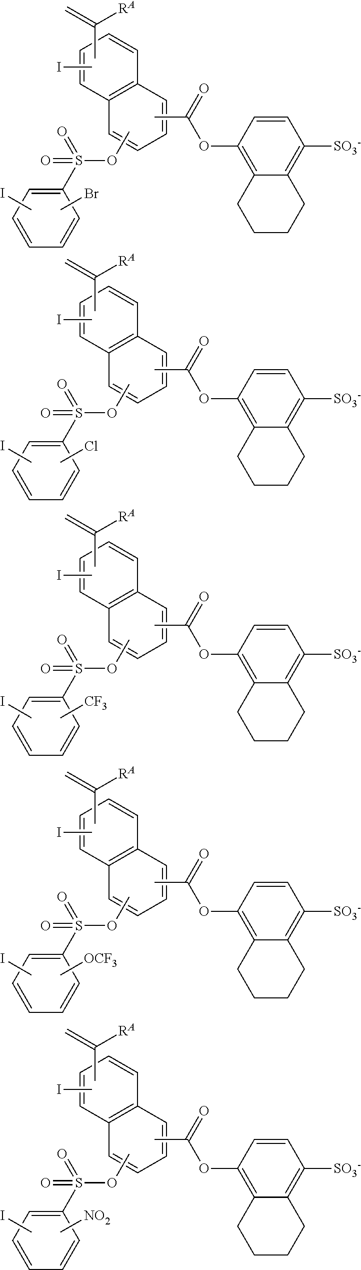

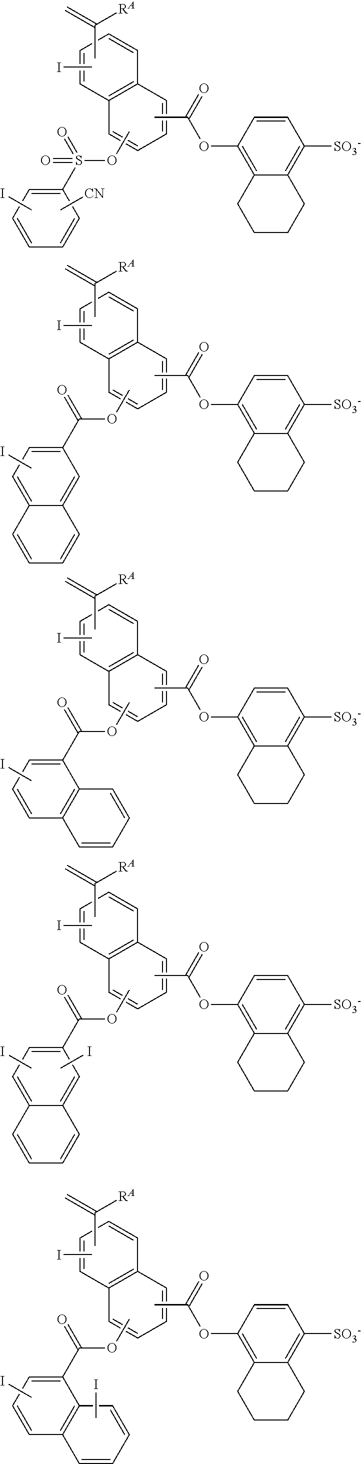

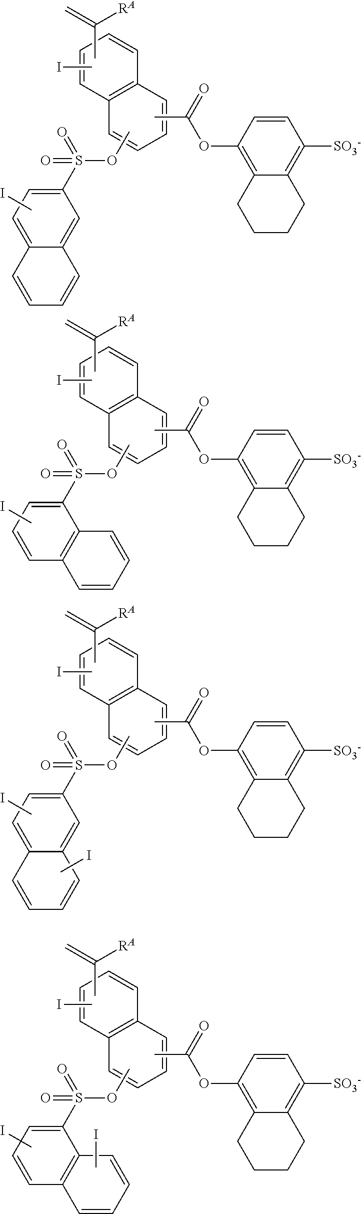

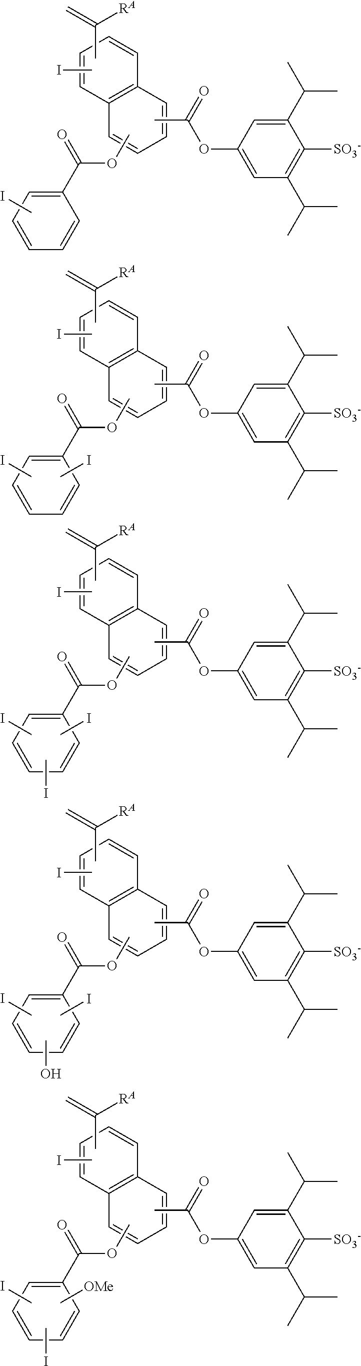

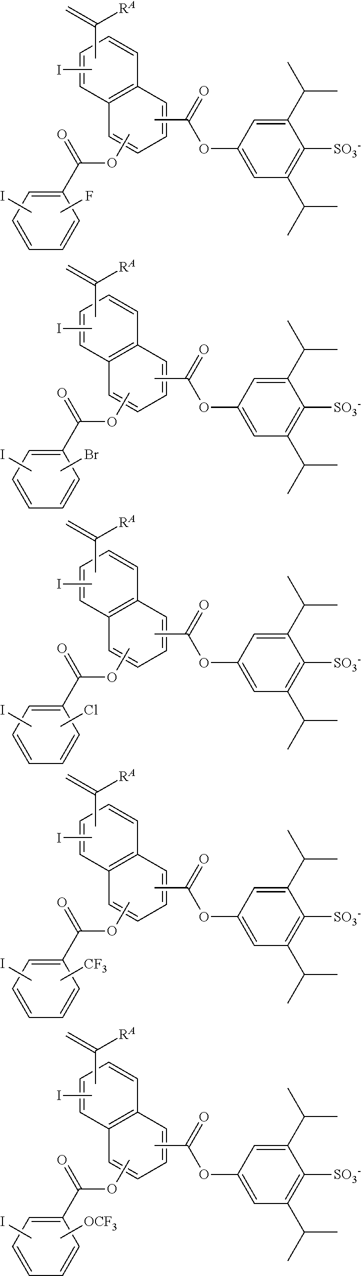

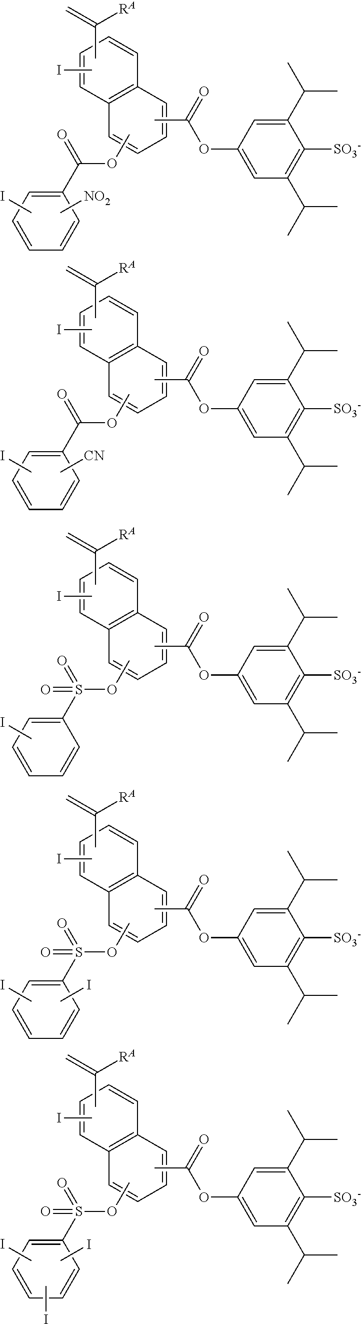

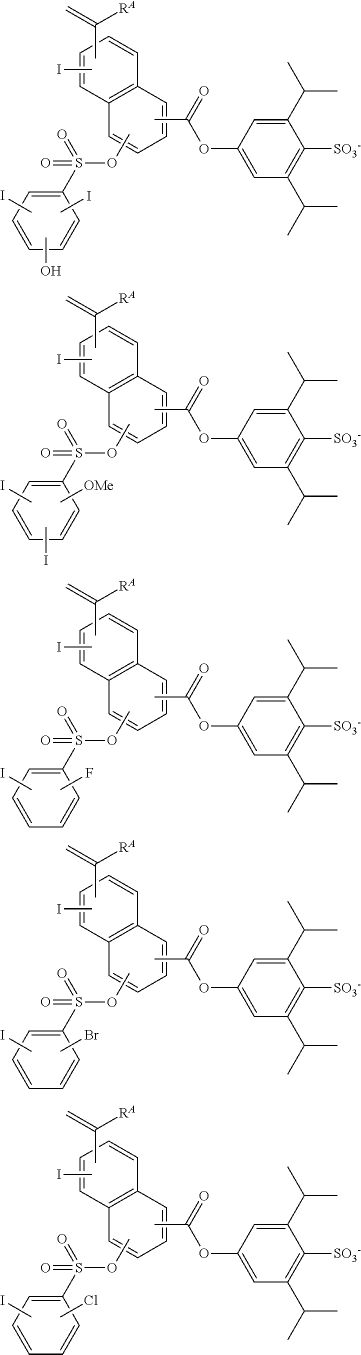

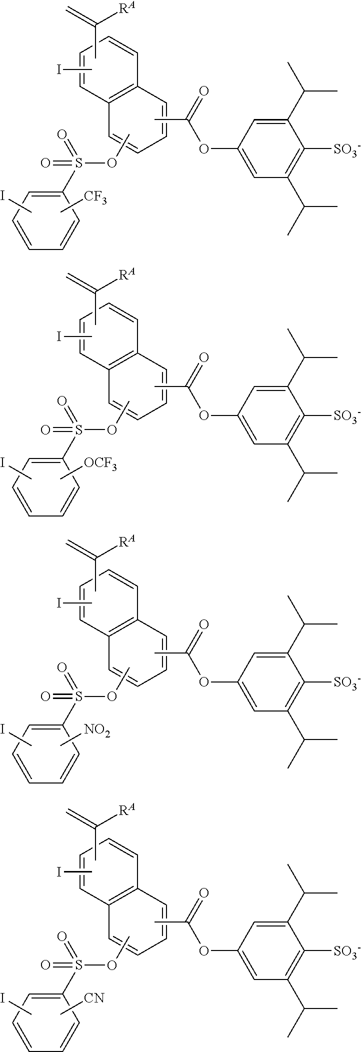

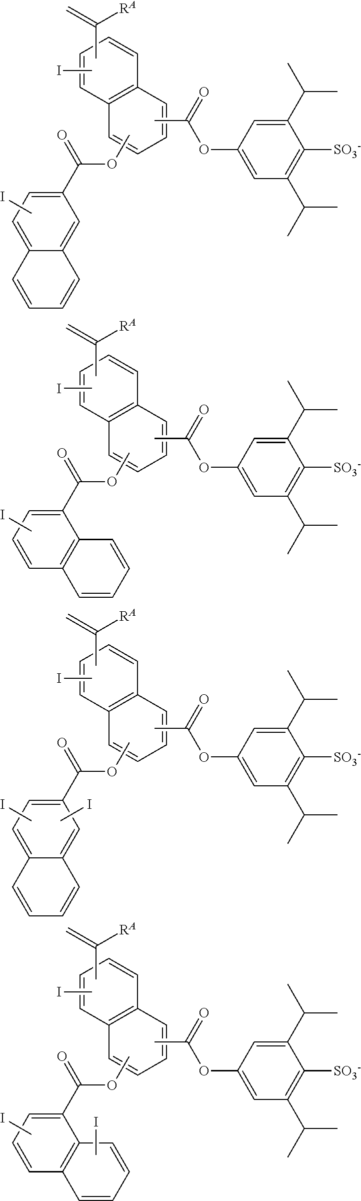

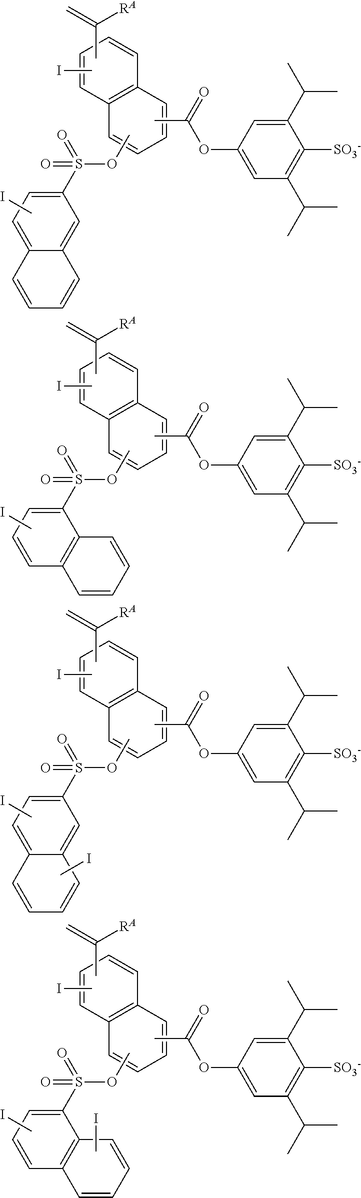

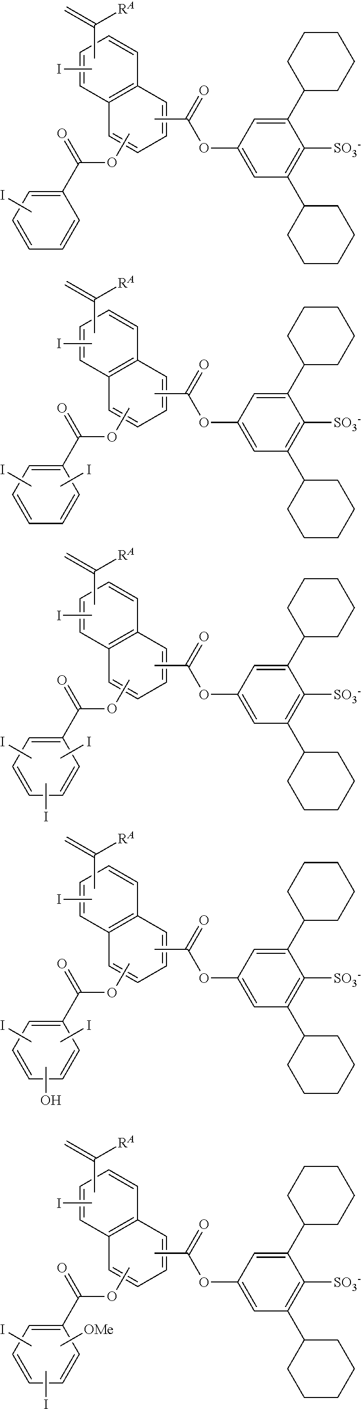

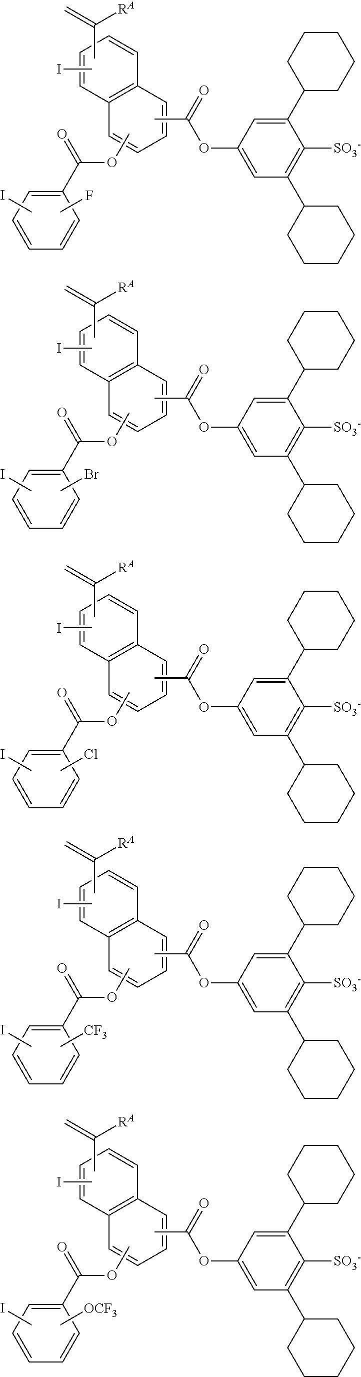

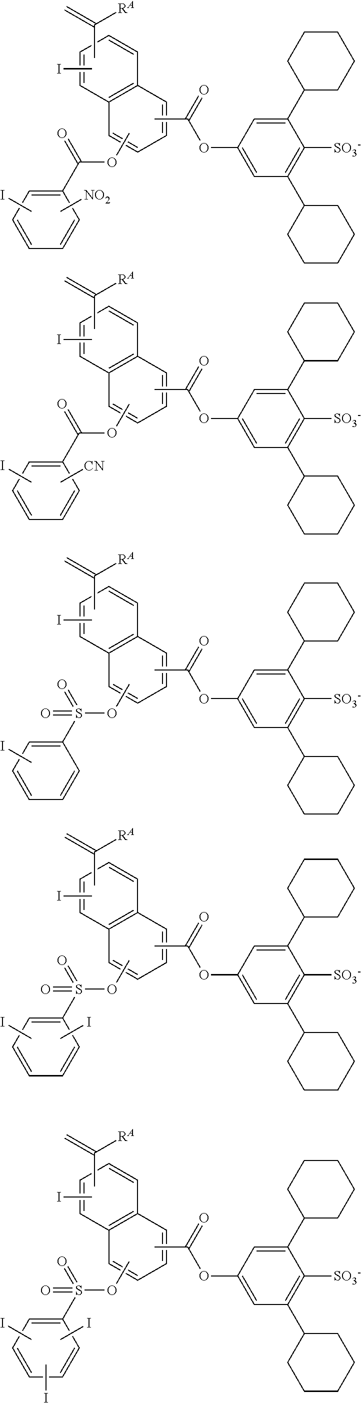

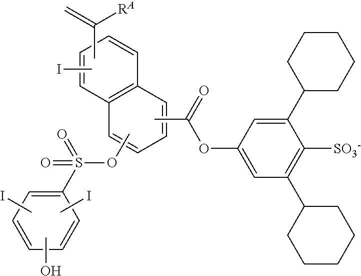

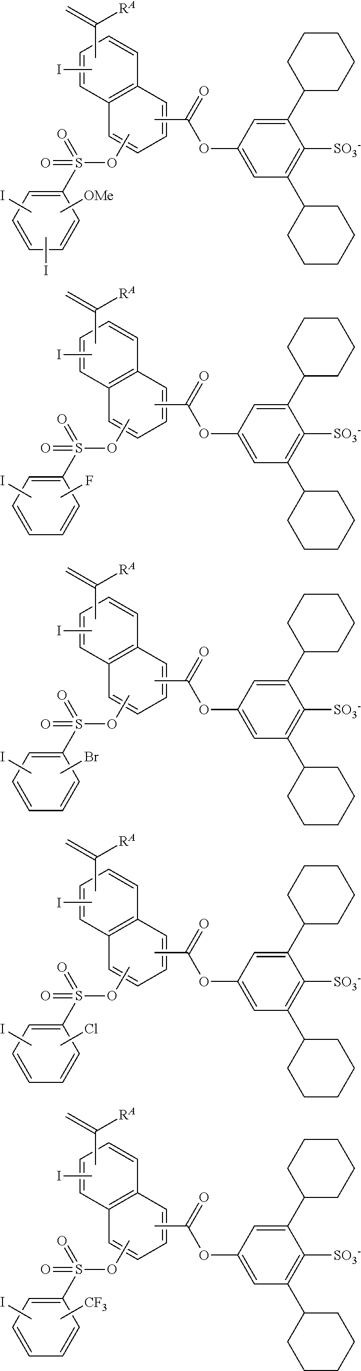

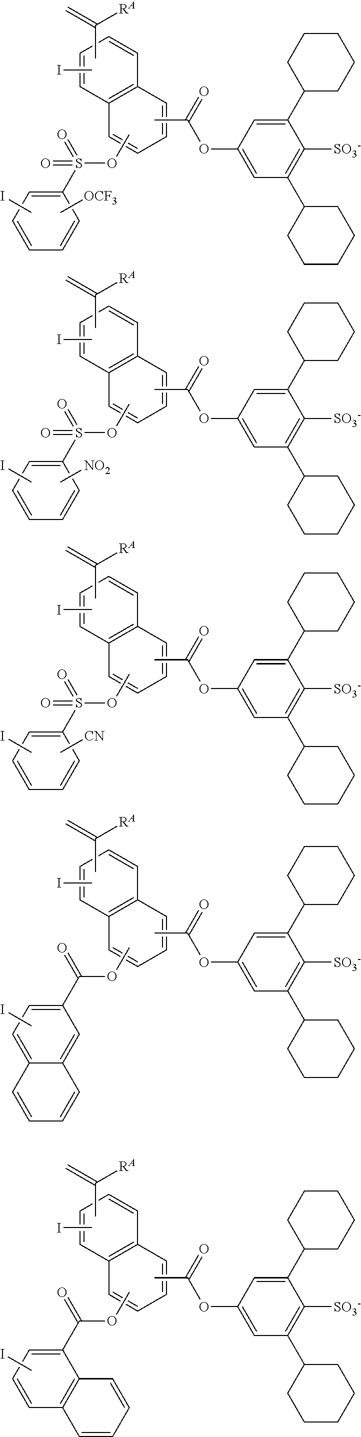

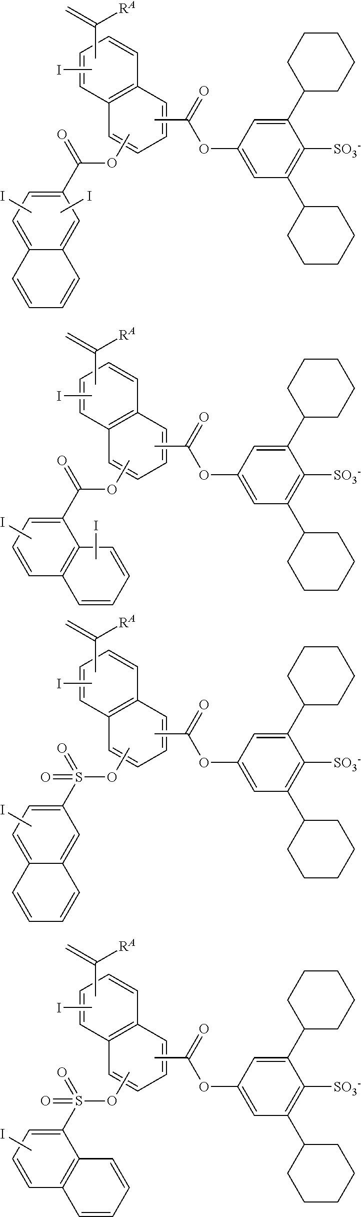

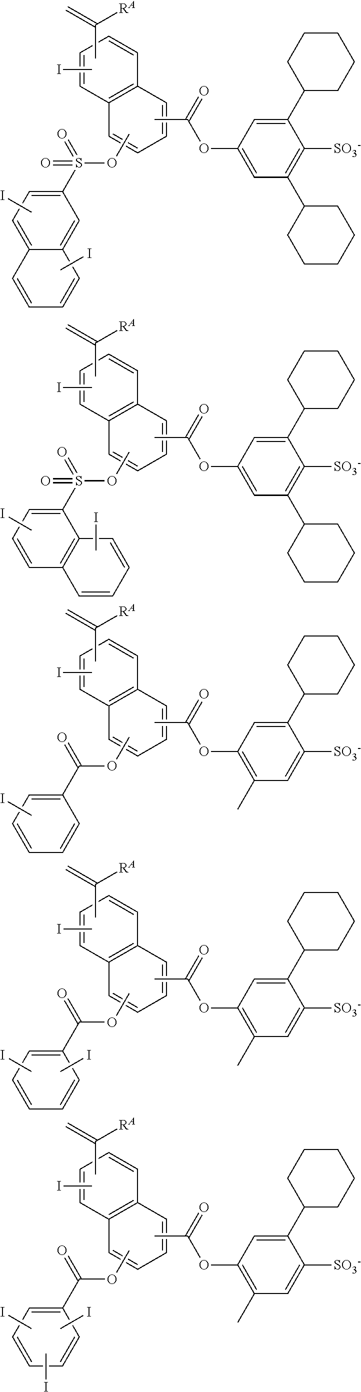

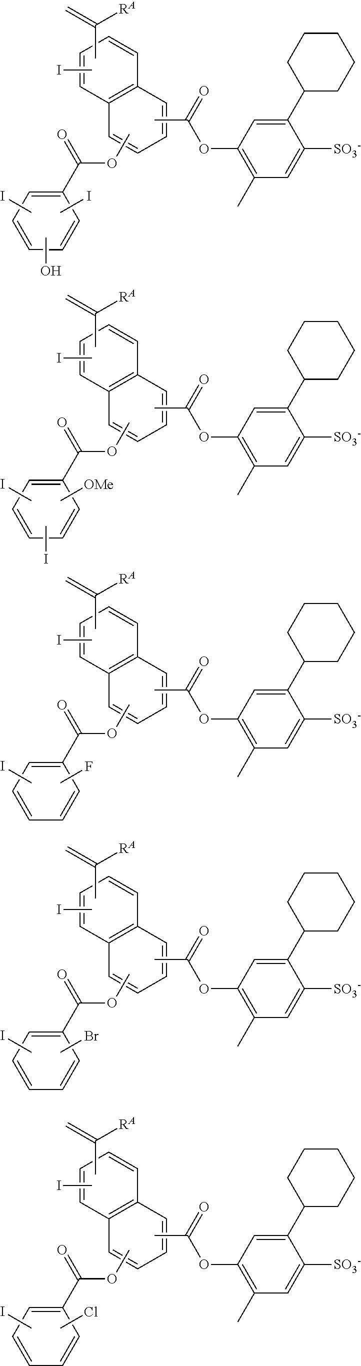

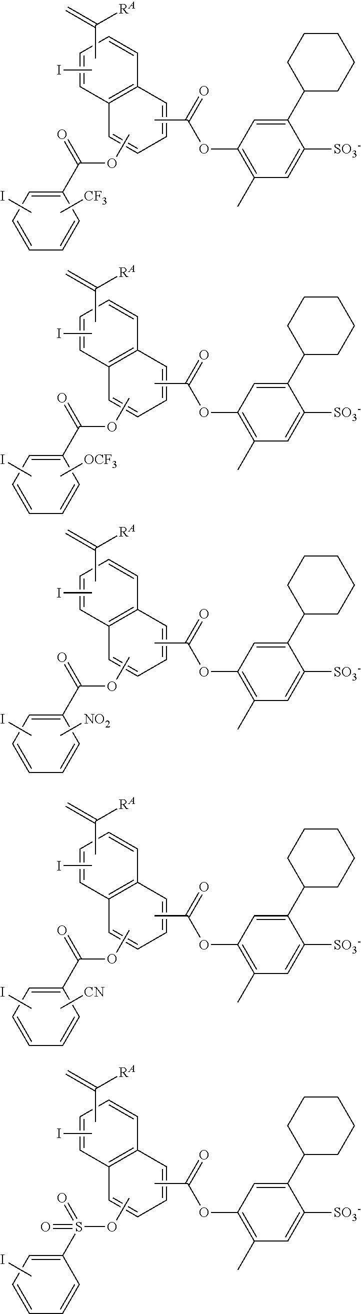

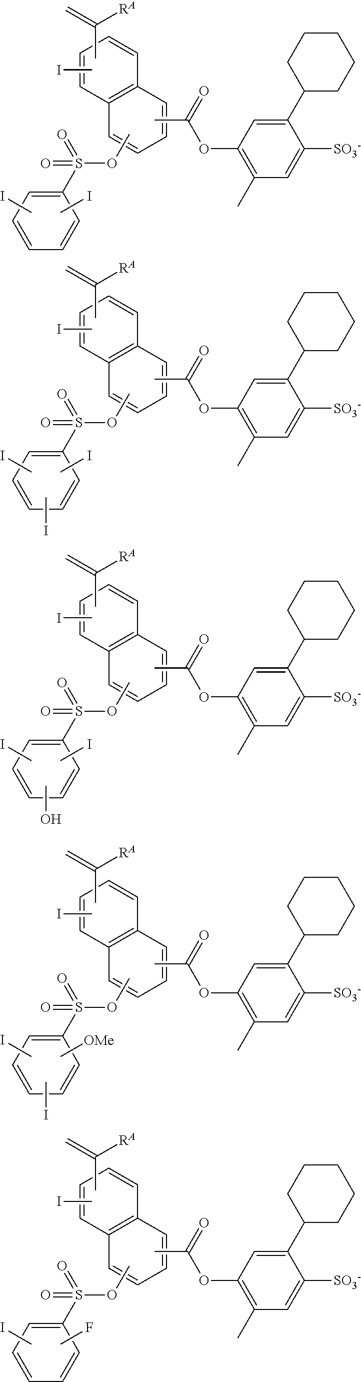

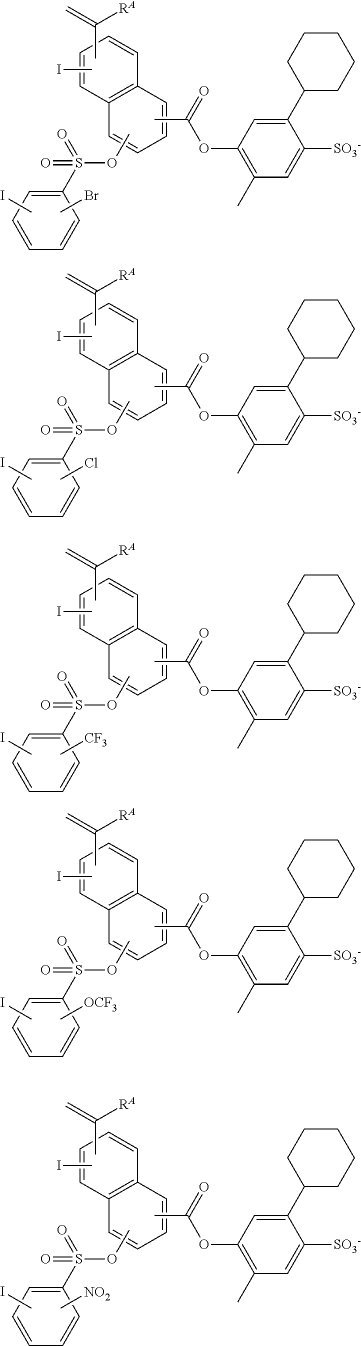

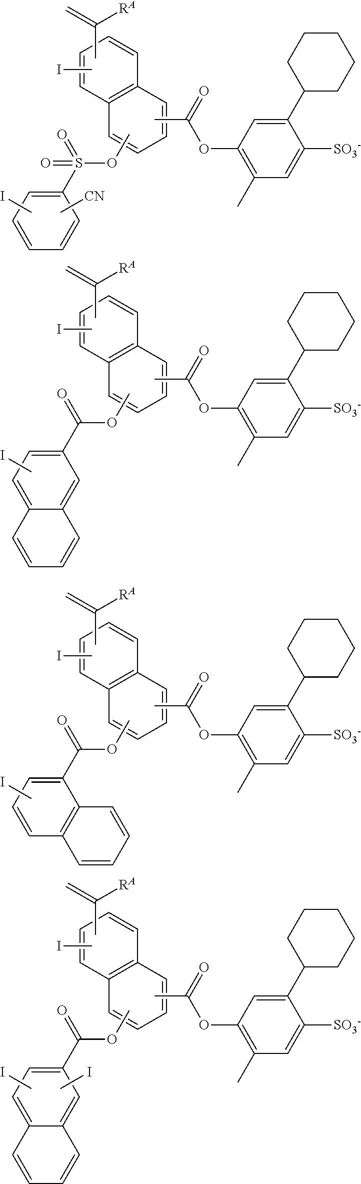

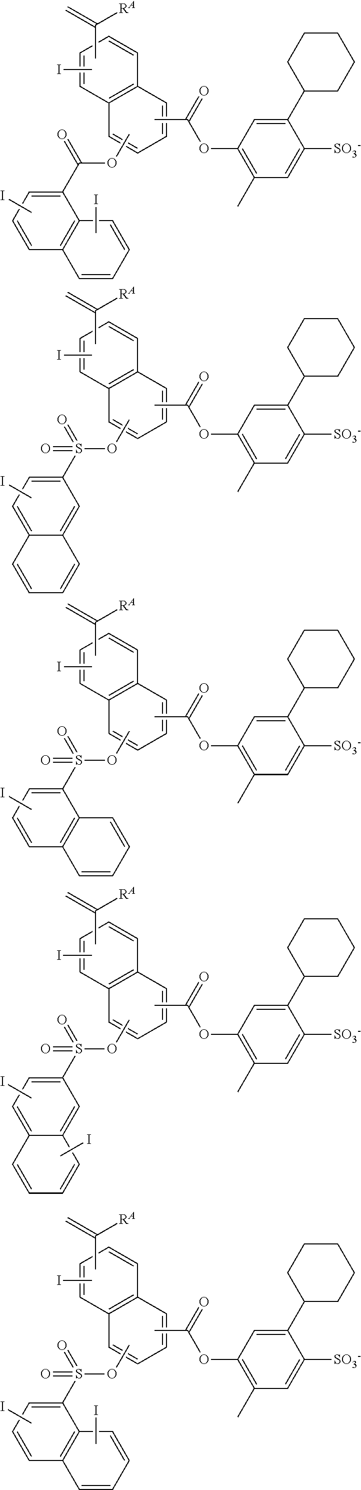

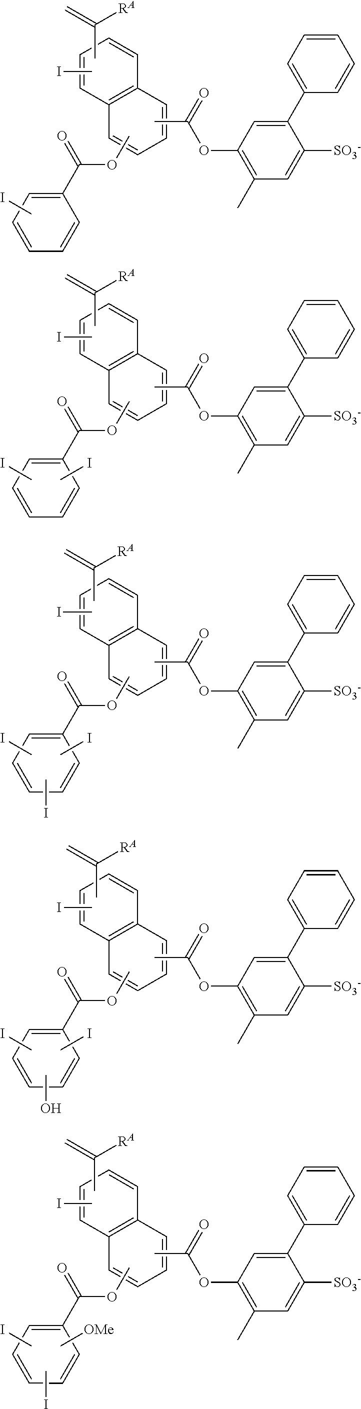

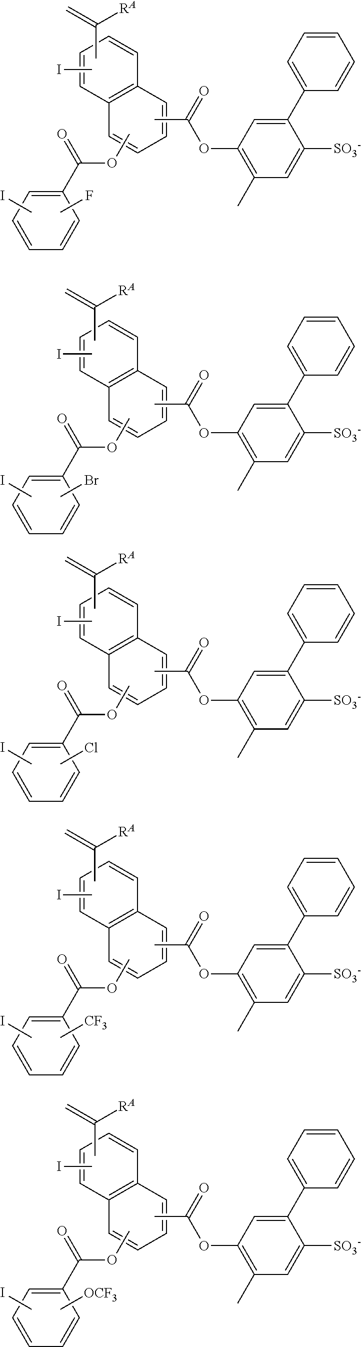

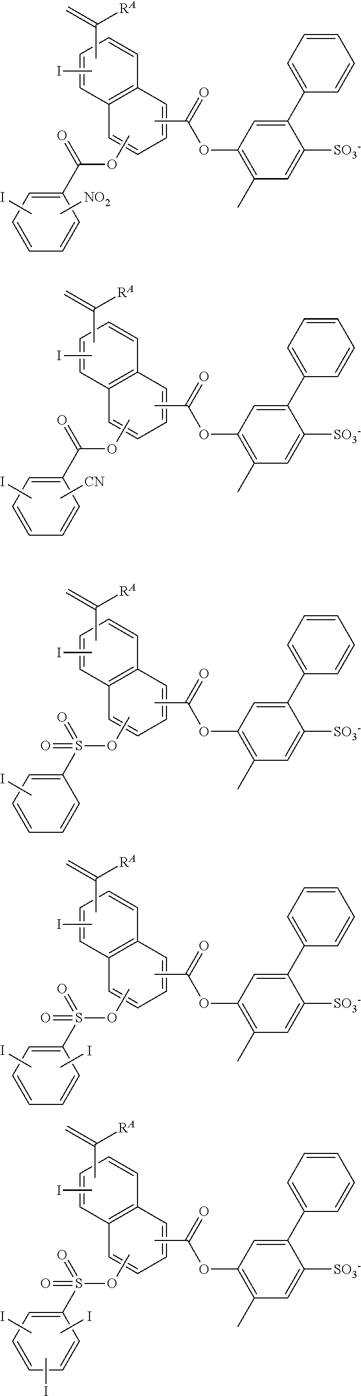

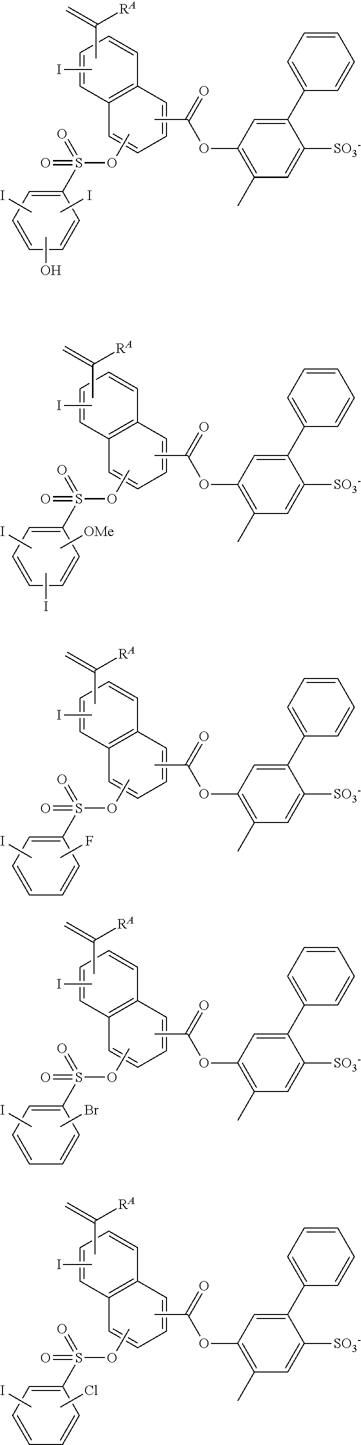

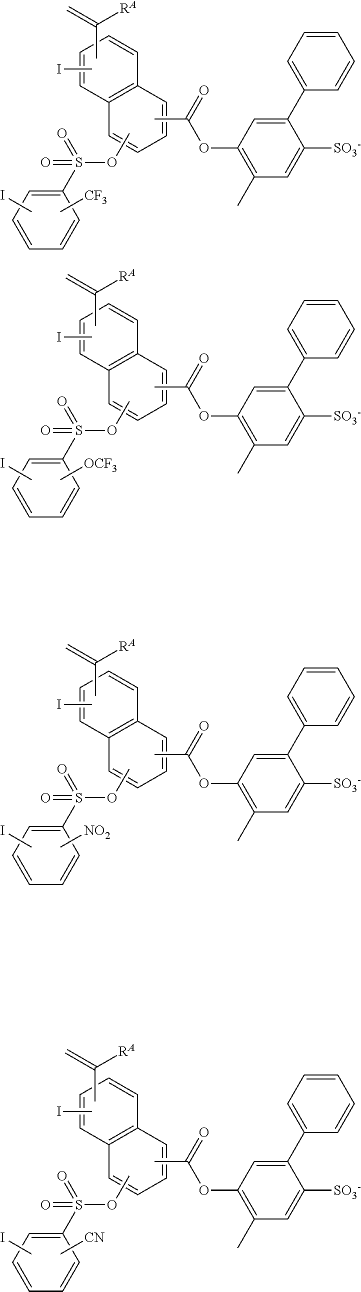

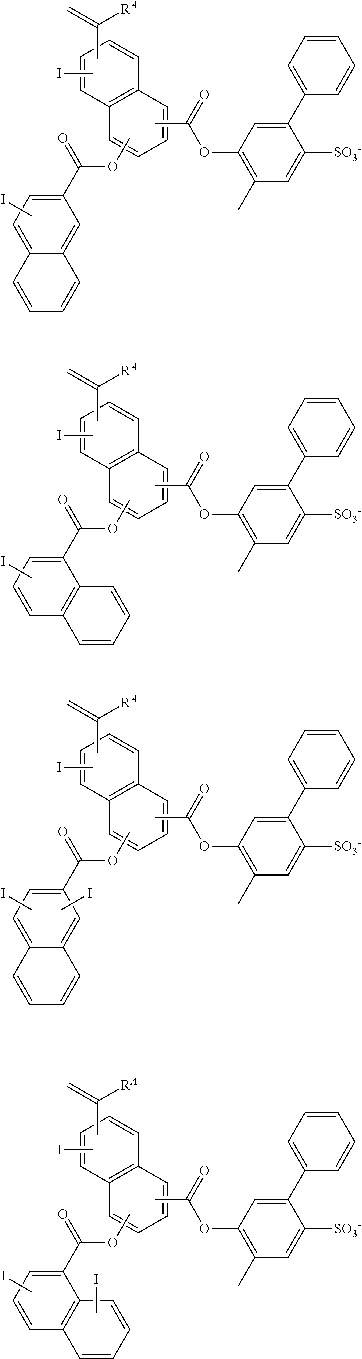

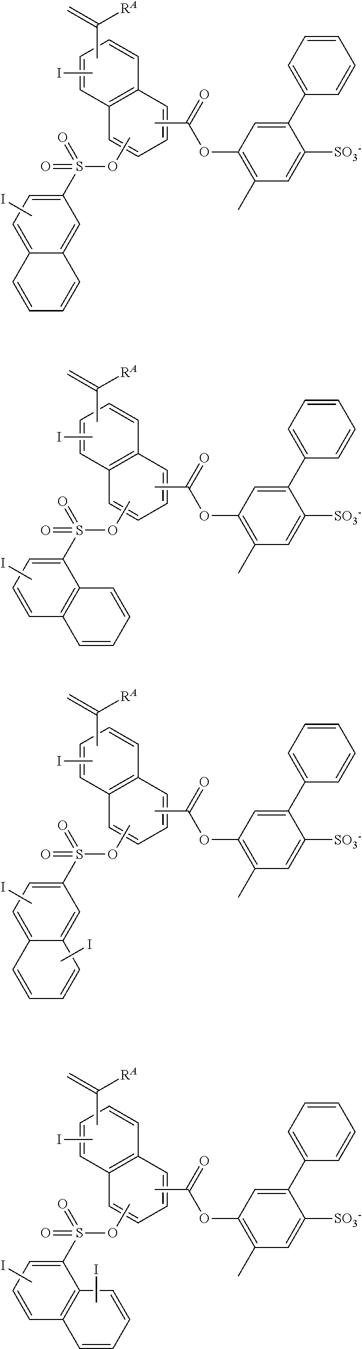

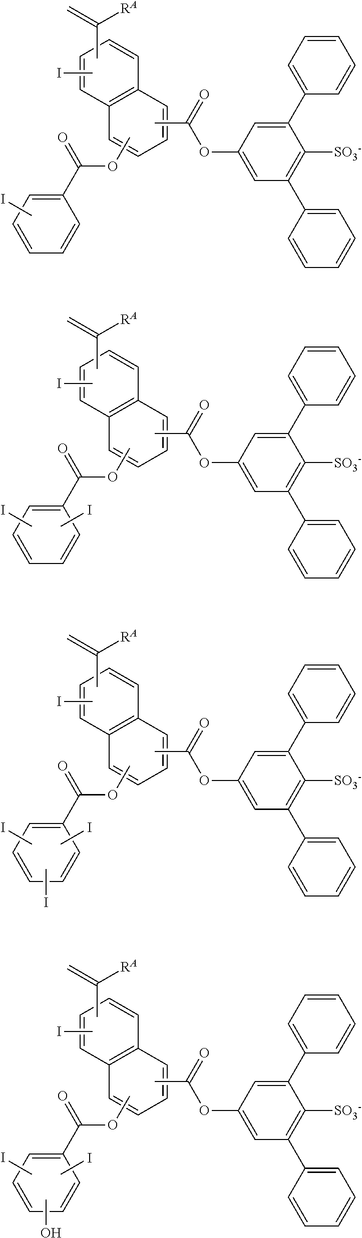

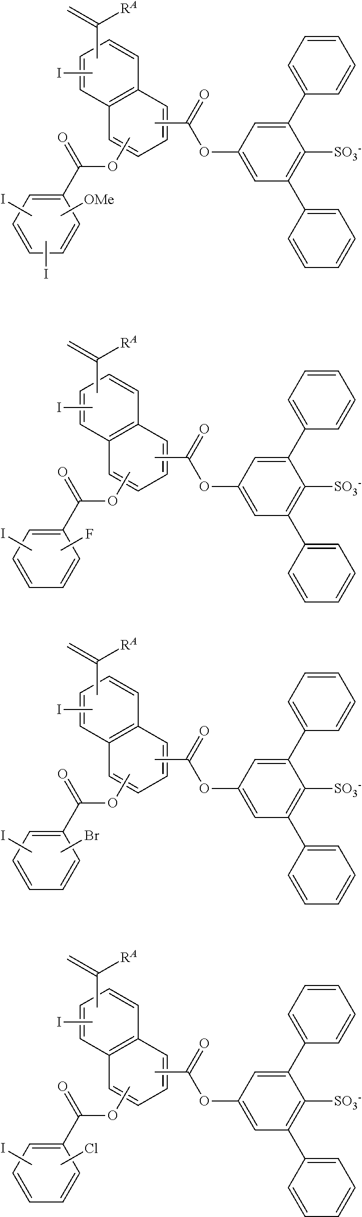

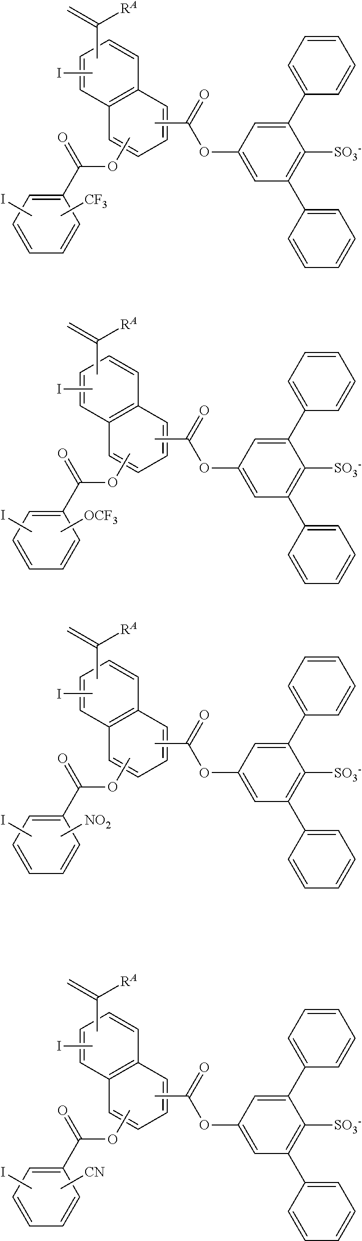

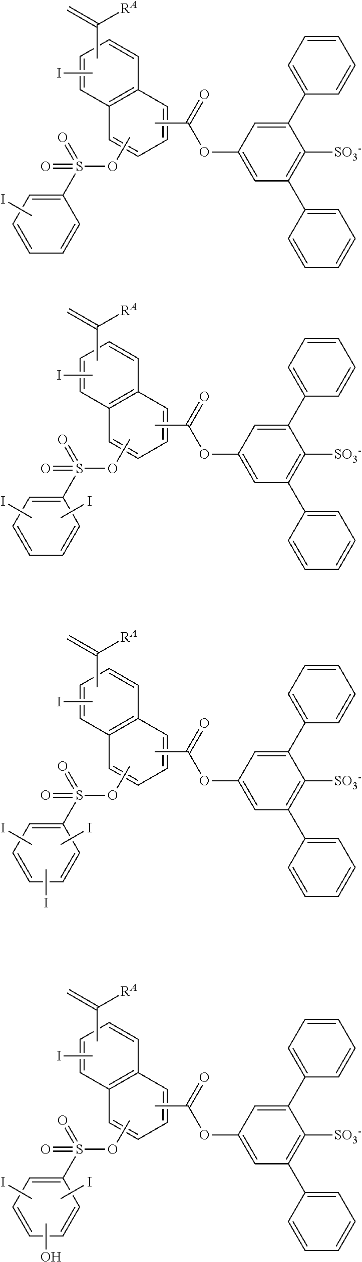

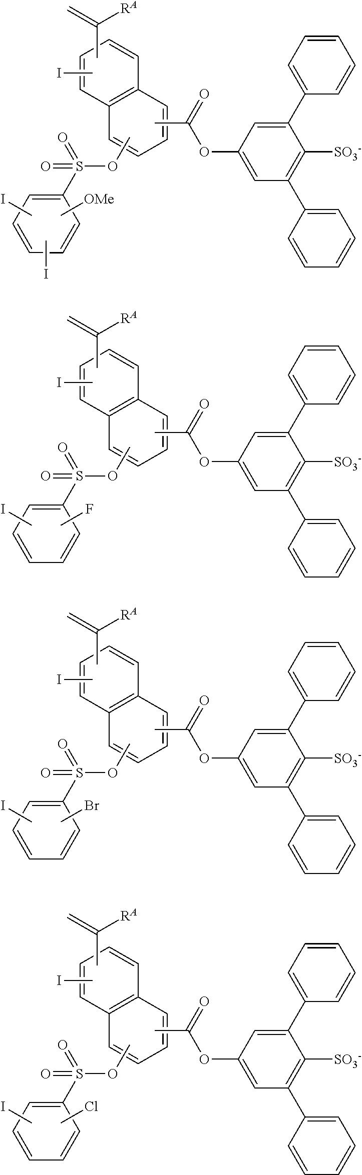

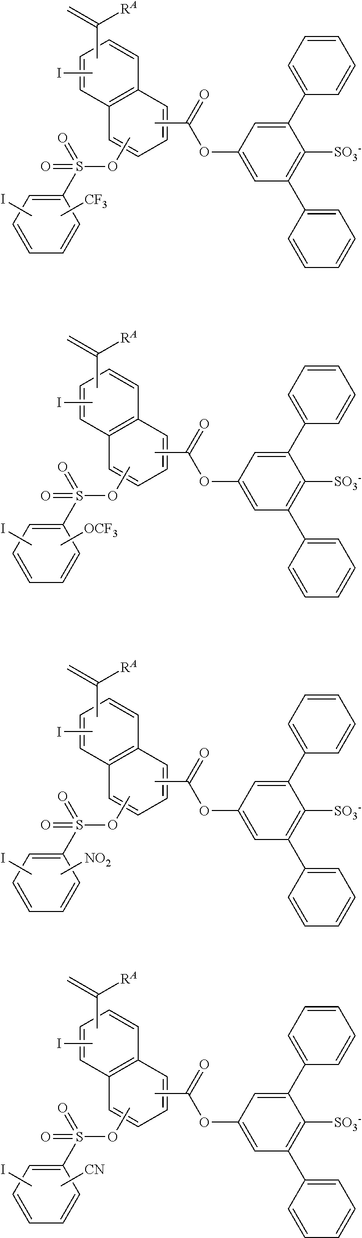

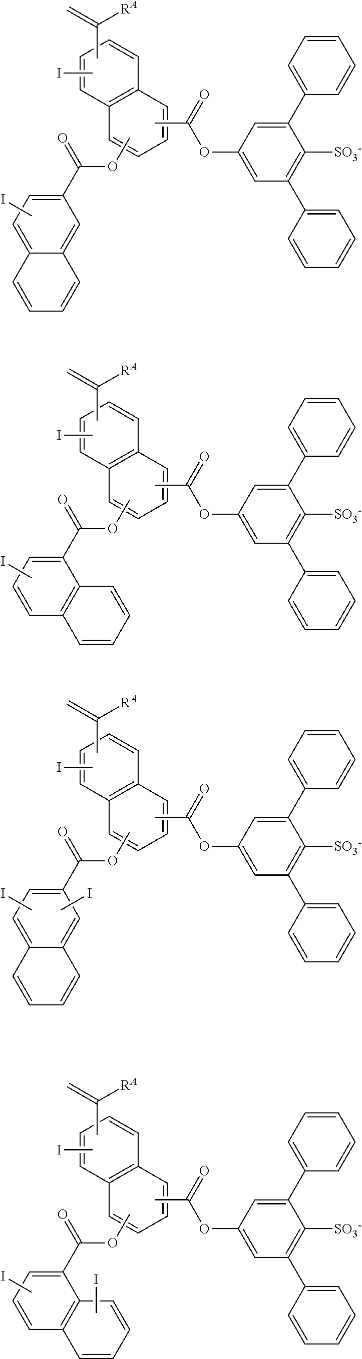

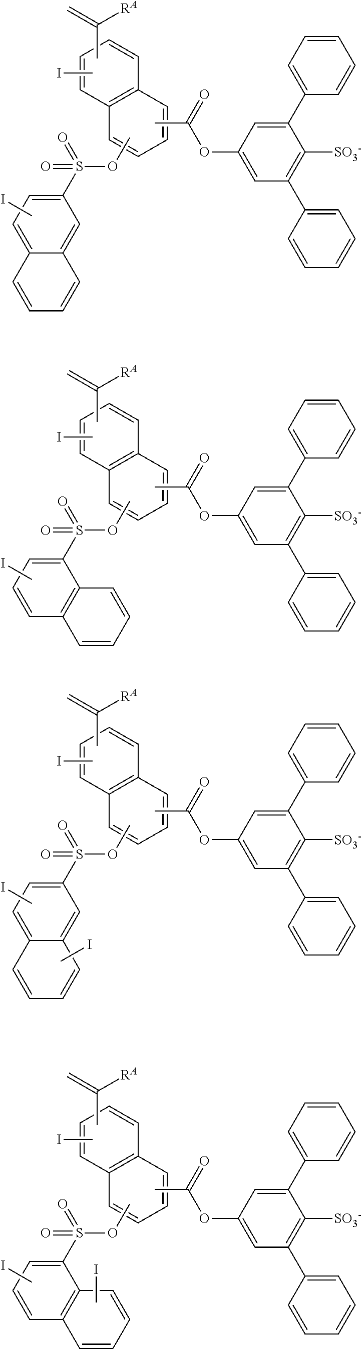

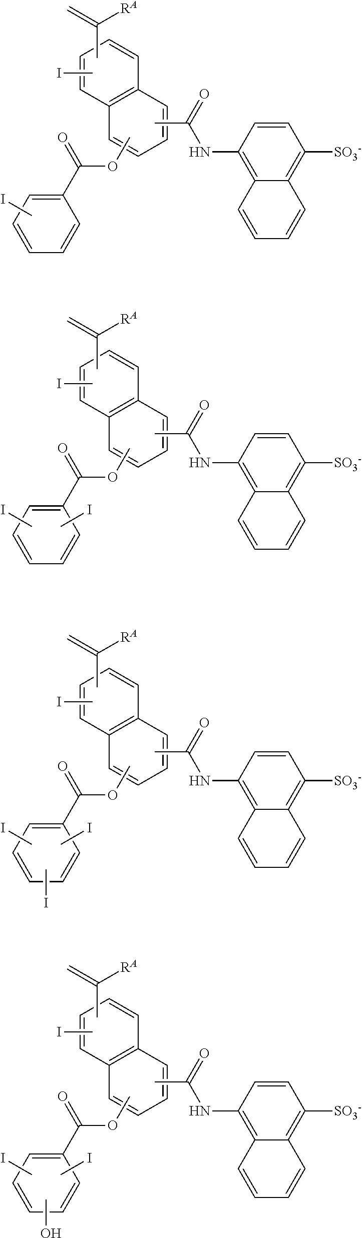

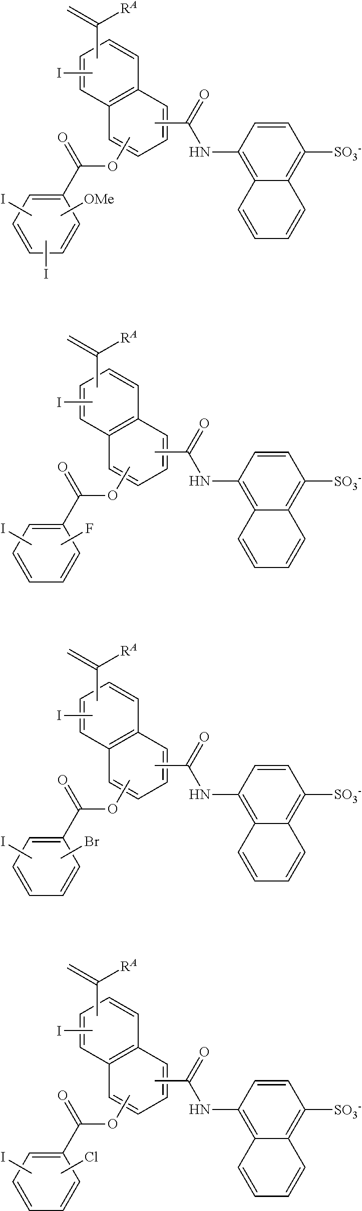

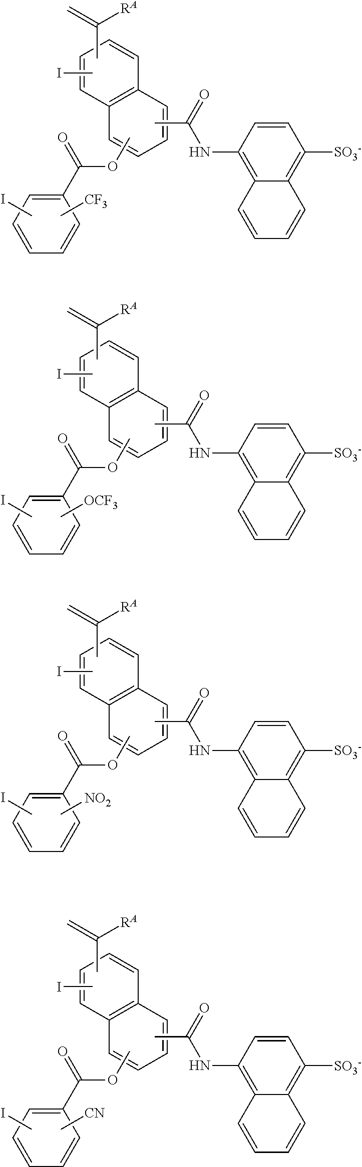

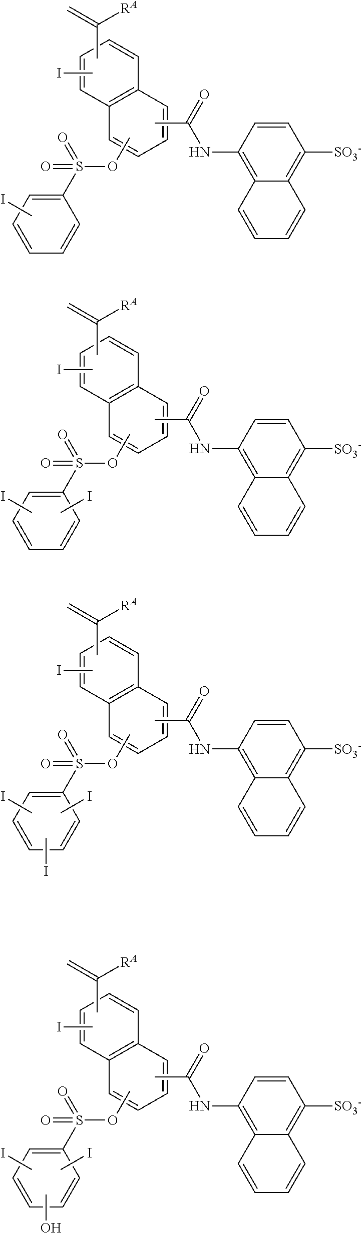

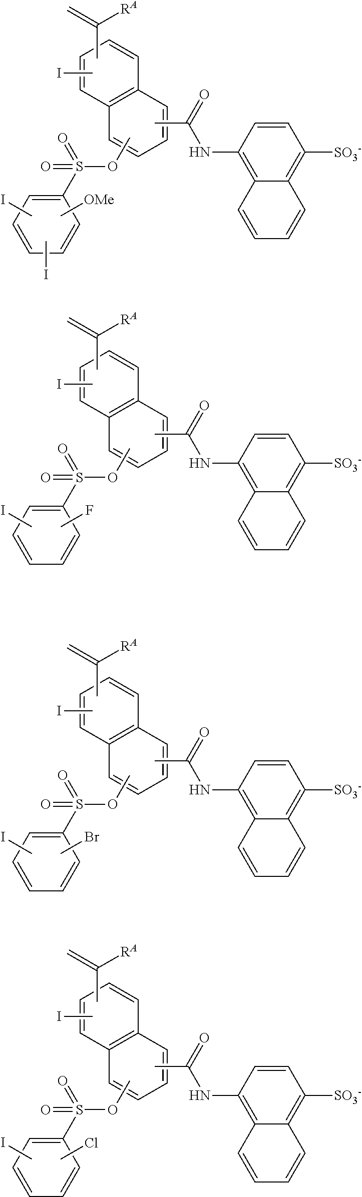

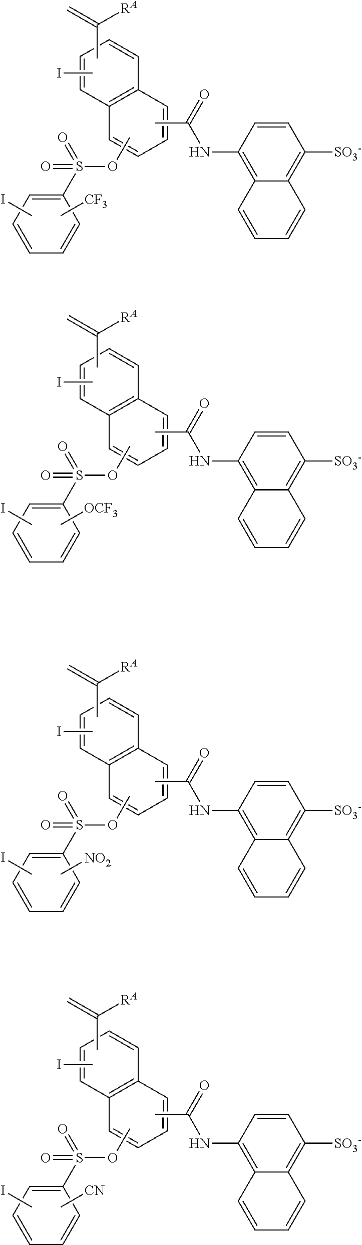

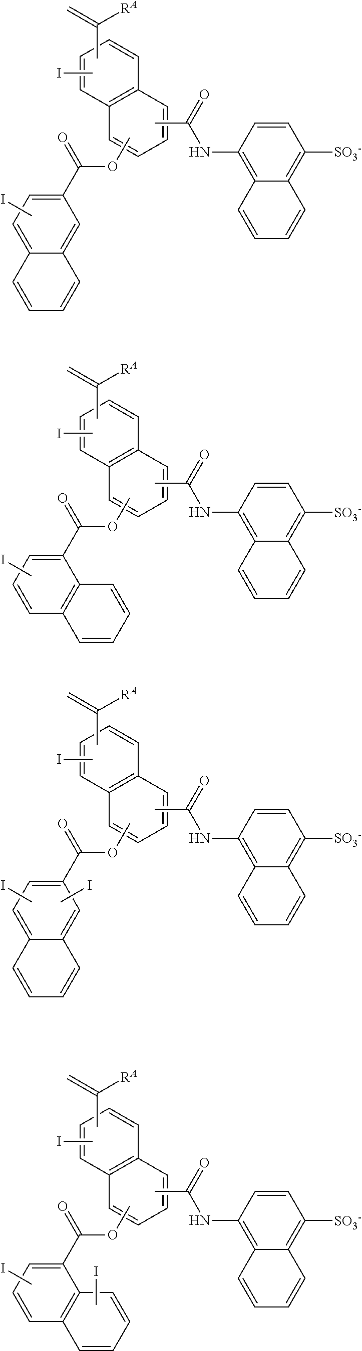

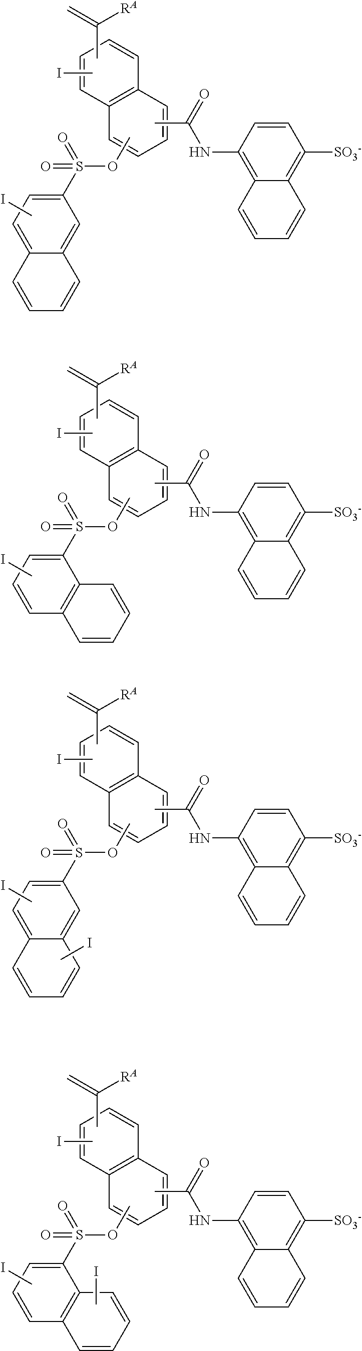

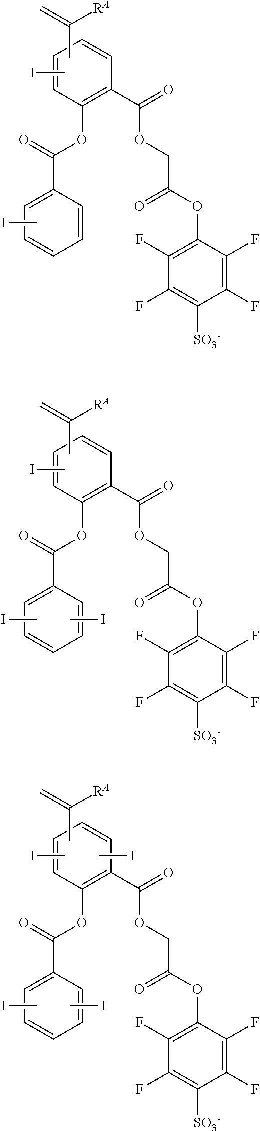

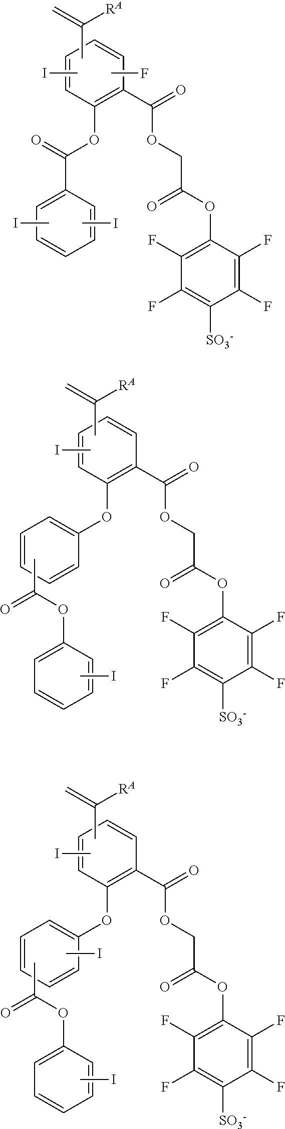

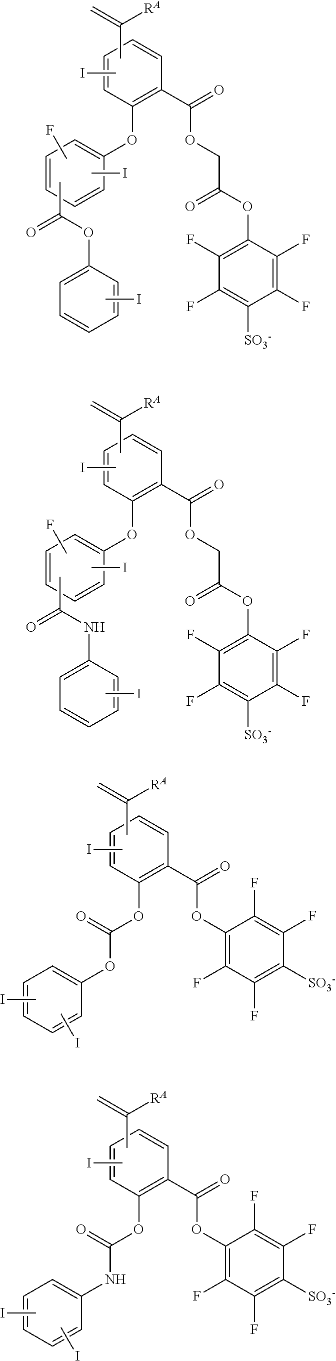

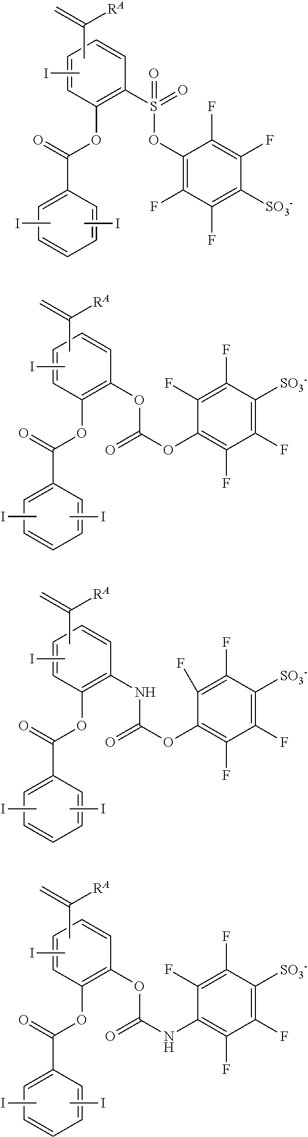

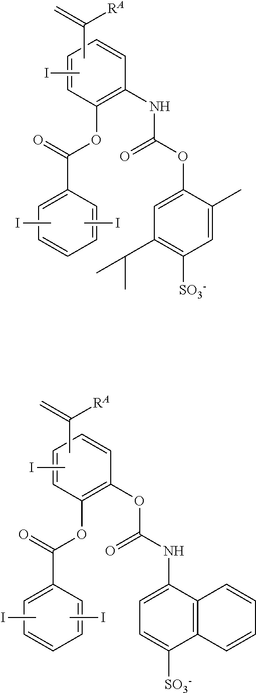

In formula (a), Z⁺ is an onium cation. The onium cation is preferably a sulfonium cation having the following formula (Z-1) or an iodonium cation having the following formula (Z-2):

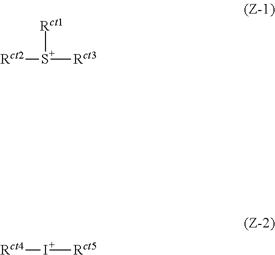

In formulas (Z-1) and (Z-2), R^{ct1} to R^{ct5} are each independently a halogen atom or a C₁-C₃₀ hydrocarbyl group which may contain a heteroatom.

Specific examples of the halogen atom of R^{ct1} to R^{ct5} include a fluorine atom, a chlorine atom, a bromine atom, and an iodine atom.

The hydrocarbyl group of R^{ct1} to R^{ct5} may be saturated or unsaturated, and may be linear, branched, or cyclic. Specific examples thereof include: a C₁-C₃₀ alkyl group such as a methyl group, an ethyl group, a n-propyl group, an isopropyl group, a n-butyl group, an isobutyl group, a sec-butyl group, or a tert-butyl group; a C₃-C₃₀ cyclic saturated hydrocarbyl group such as a cyclopropyl group, a cyclopentyl group, a cyclohexyl group, a cyclopropylmethyl group, a 4-methylcyclohexyl group, a cyclohexylmethyl group, a norbornyl group, or an adamantyl group; a C₂-C₃₀ alkenyl group such as a vinyl group, an allyl group, a propenyl group, a butenyl group, or a hexenyl group; a C₃-C₃₀ cyclic unsaturated hydrocarbyl group such as a cyclohexenyl group; a C₆-C₃₀ aryl group such as a phenyl group, a naphthyl group, or a thienyl group; a C₇-C₃₀ aralkyl group such as a benzyl group, a 1-phenylethyl group, or a 2-phenylethyl group; and a group obtained by combining these, and an aryl group is preferable. Some or all of hydrogen atoms of the hydrocarbyl group may be replaced with a group containing a heteroatom such as an oxygen atom, a sulfur atom, a nitrogen atom, or a halogen atom, some of (—CH₂—)s in the hydrocarbyl group may be replaced with a group containing a heteroatom such as an oxygen atom, a sulfur atom, or a nitrogen atom, and as a result, the hydrocarbyl group may contain a hydroxy group, a fluorine atom, a chlorine atom, a bromine atom, an iodine atom, a cyano group, a nitro group, a carbonyl group, an ether bond, an ester bond, a sulfonic acid ester bond, a carbonate bond, a lactone ring, a sultone ring, a carboxylic acid anhydride (—C(═O)—O—C(═O)—), a haloalkyl group, or the like.

R^{ct1} and R^{ct2} may be bonded to each other to form a ring together with a sulfur atom to which they are bonded. At this time, specific examples of the structure of the ring include those having the following formulas:

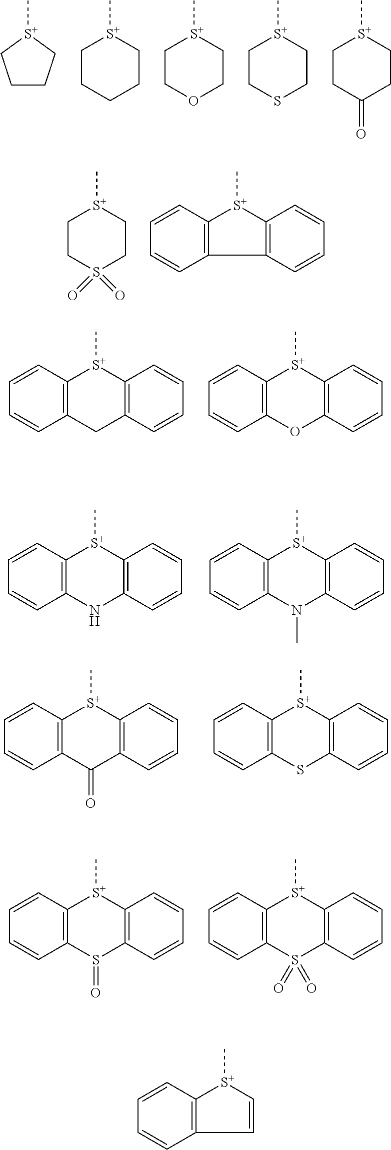

wherein the dashed line is a bond with R^{ct3}.

Specific examples of the sulfonium cation having formula (Z-1) include, but are not limited to, those listed below.

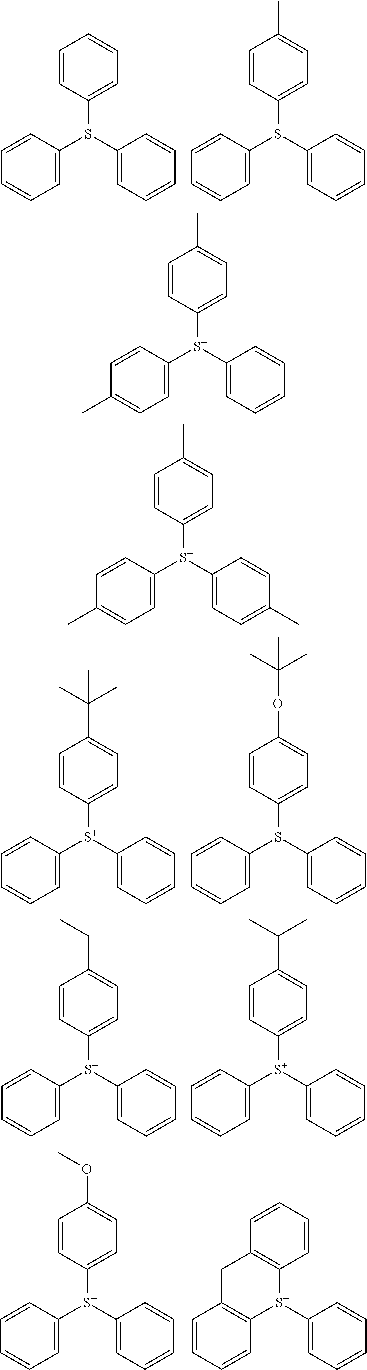

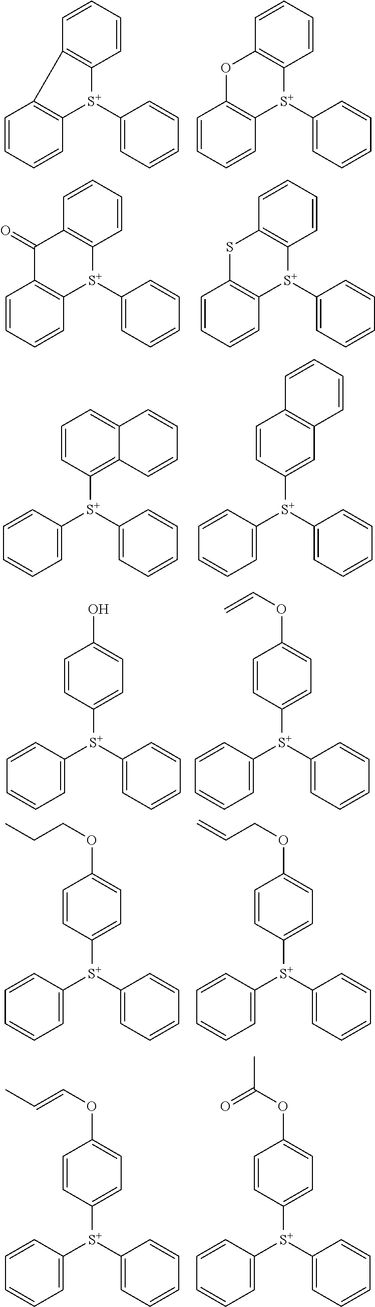

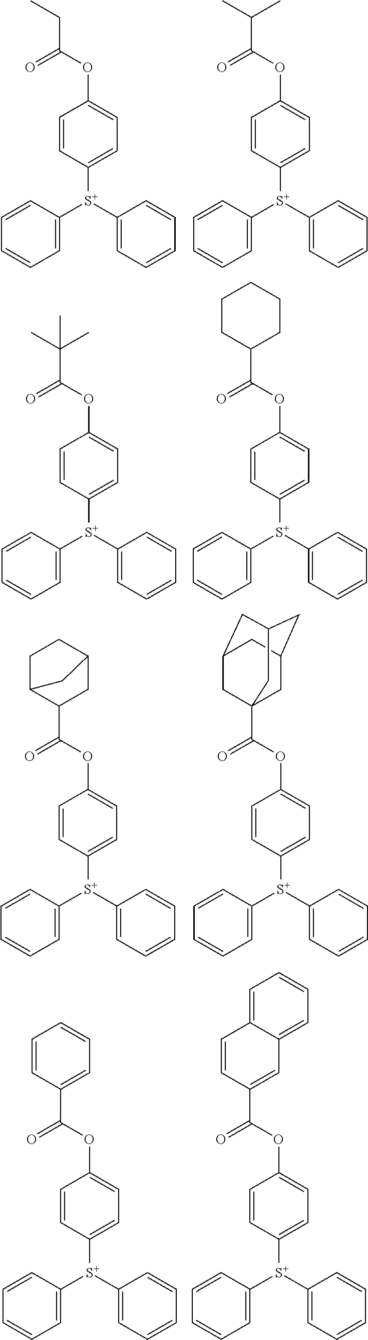

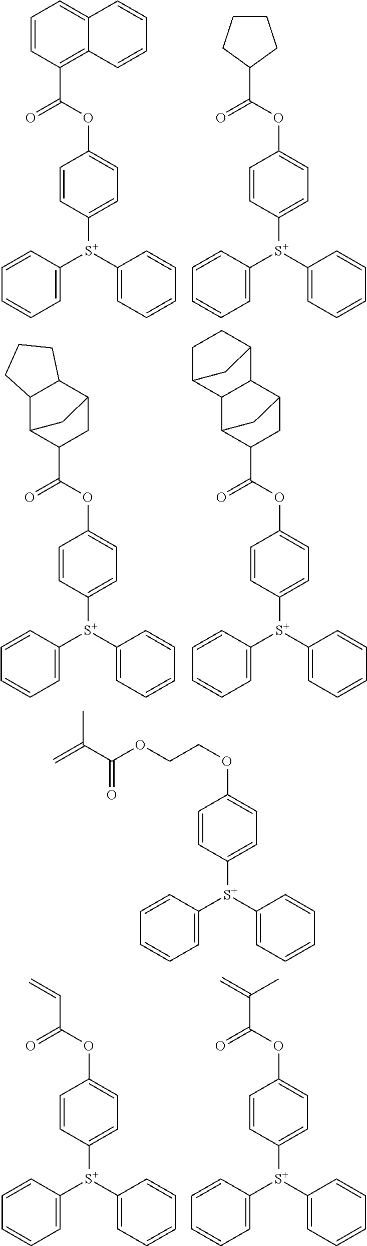

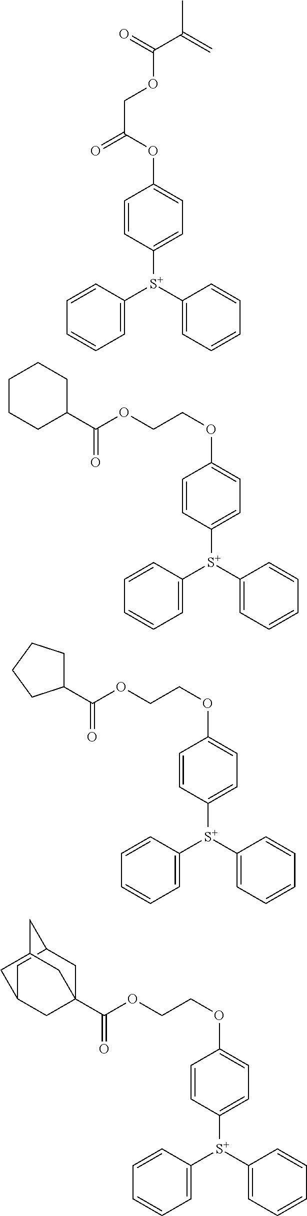 [SMILES: C=C(C)C(=O)OCC(=O)Oc1ccc([S+](c2ccccc2)c2ccccc2)cc1 ; O=C(OCCOc1ccc([S+](c2ccccc2)c2ccccc2)cc1)C1CCCCC1 ; O=C(OCCOc1ccc([S+](c2ccccc2)c2ccccc2)cc1)C1CCCC1 ; O=C(OCCOc1ccc([S+](c2ccccc2)c2ccccc2)cc1)C12CC3CC(CC(C3)C1)C2]

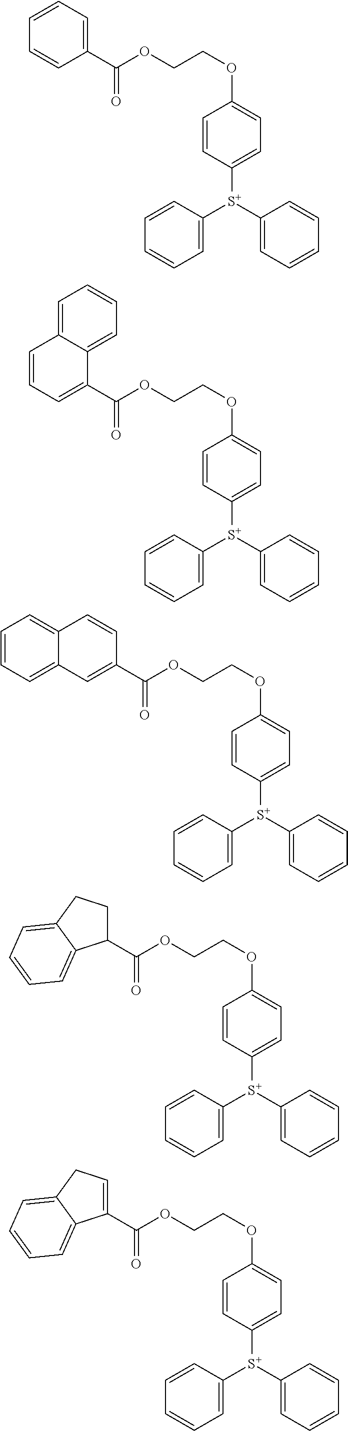 [SMILES: O=C(OCCOc1ccc([S+](c2ccccc2)c2ccccc2)cc1)c1ccccc1 ; O=C(OCCOc1ccc([S+](c2ccccc2)c2ccccc2)cc1)c1cccc2ccccc12 ; O=C(OCCOc1ccc([S+](c2ccccc2)c2ccccc2)cc1)c1ccc2ccccc2c1 ; O=C(OCCOc1ccc([S+](c2ccccc2)c2ccccc2)cc1)C1CCc2ccccc21 ; O=C(OCCOc1ccc([S+](c2ccccc2)c2ccccc2)cc1)C1=CCc2ccccc21]

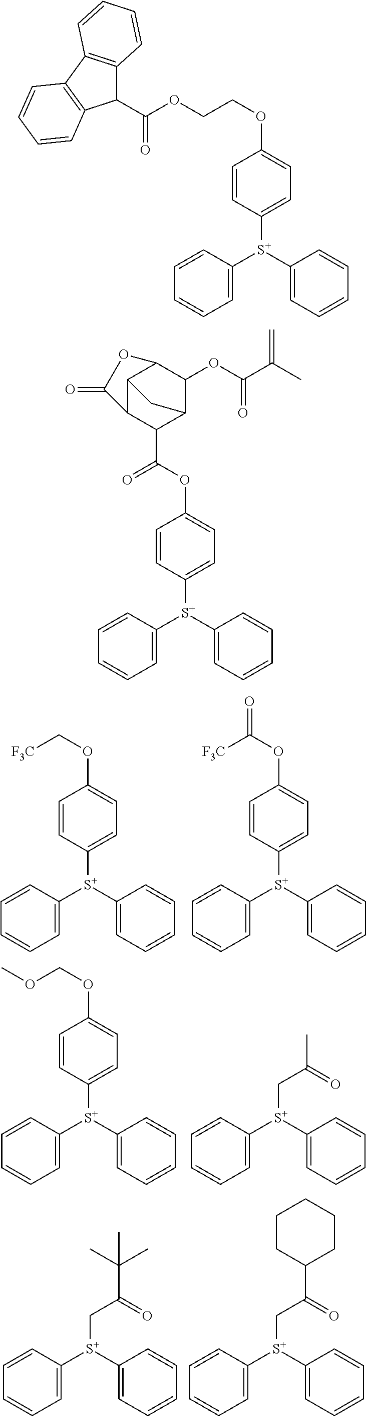

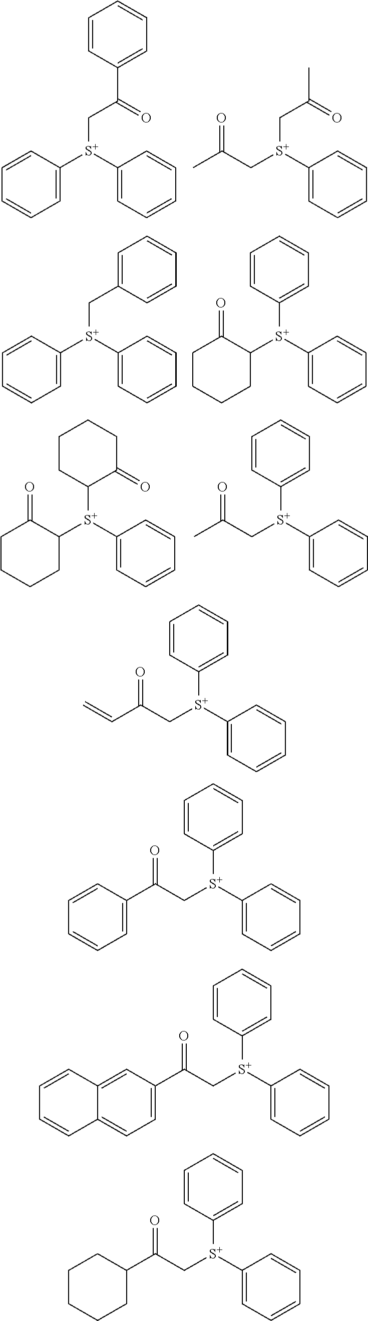

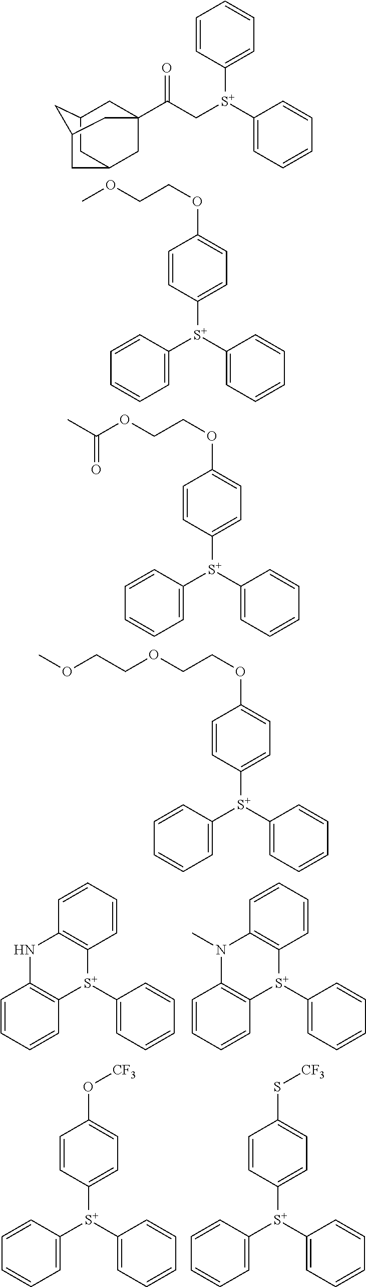

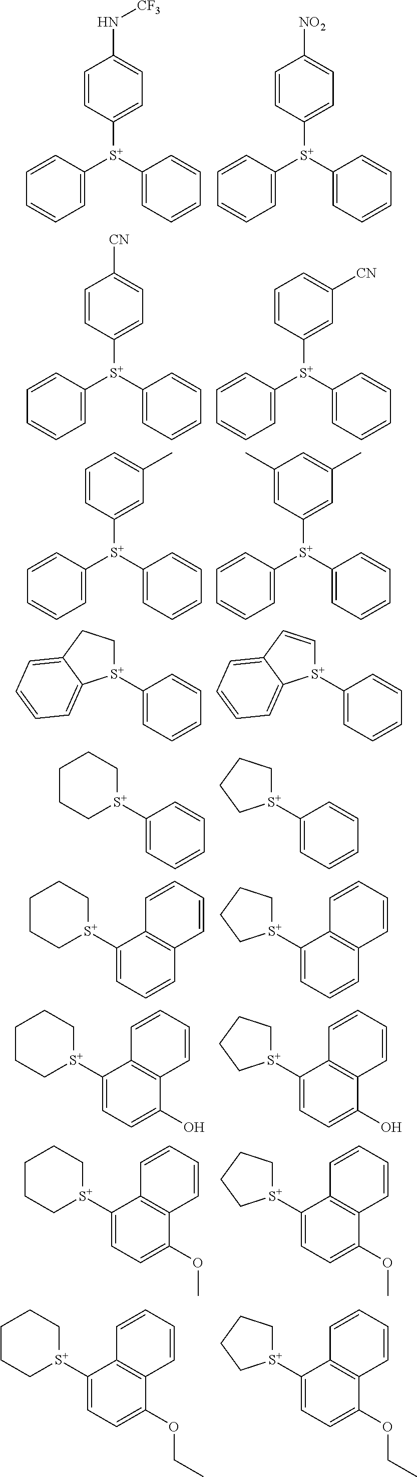

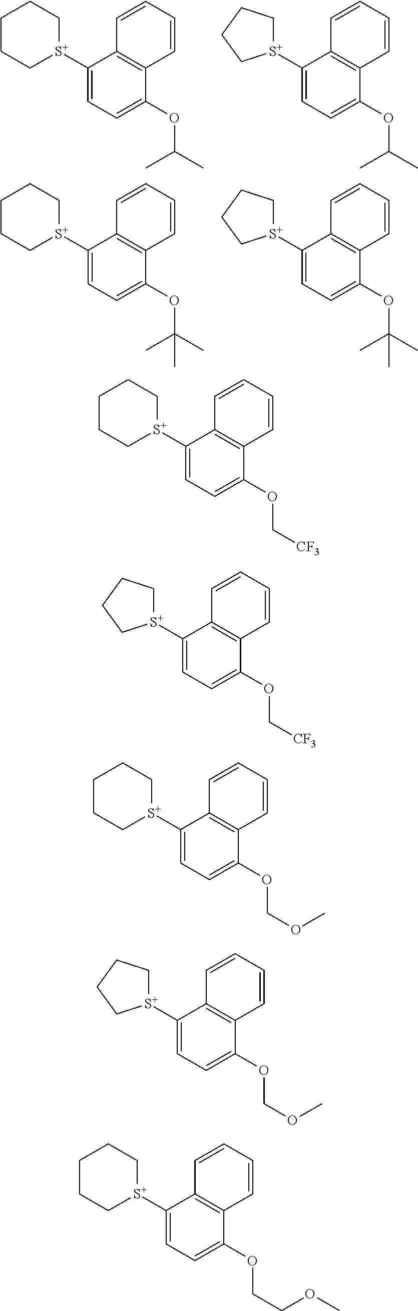

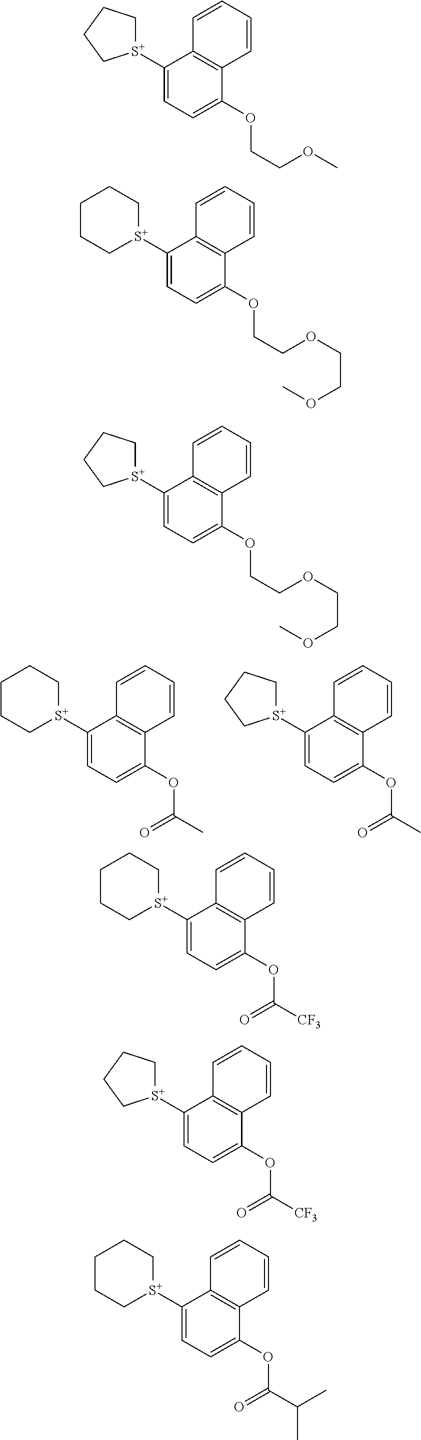

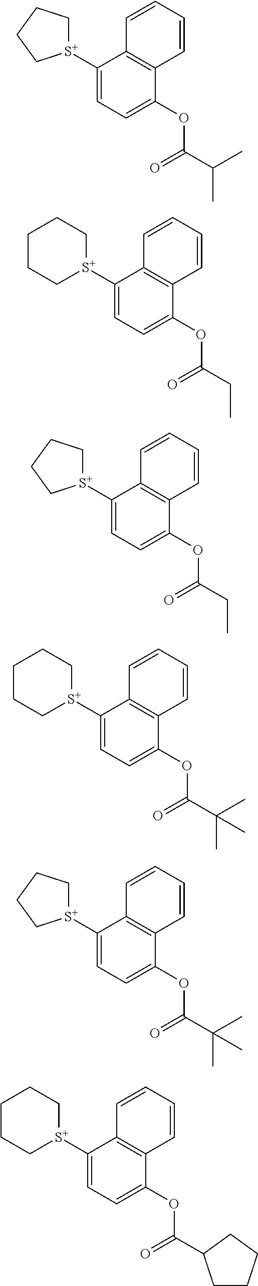 [SMILES: CC(C)C(=O)Oc1ccc([S+]2CCCC2)c2ccccc12 ; CCC(=O)Oc1ccc([S+]2CCCCC2)c2ccccc12 ; CCC(=O)Oc1ccc([S+]2CCCC2)c2ccccc12 ; CC(C)(C)C(=O)Oc1ccc([S+]2CCCCC2)c2ccccc12 ; CC(C)(C)C(=O)Oc1ccc([S+]2CCCC2)c2ccccc12 ; O=C(Oc1ccc([S+]2CCCCC2)c2ccccc12)C1CCCC1]

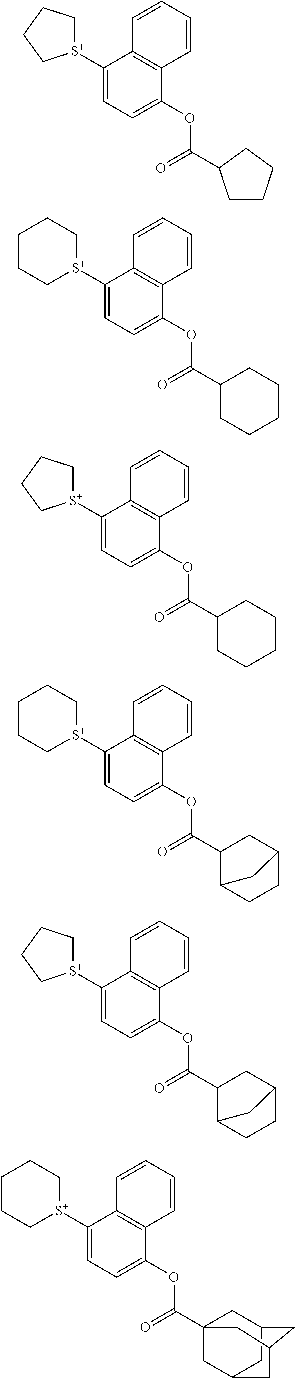 [SMILES: O=C(Oc1ccc([S+]2CCCC2)c2ccccc12)C1CCCC1 ; O=C(Oc1ccc([S+]2CCCCC2)c2ccccc12)C1CCCCC1 ; O=C(Oc1ccc([S+]2CCCC2)c2ccccc12)C1CCCCC1 ; O=C(Oc1ccc([S+]2CCCCC2)c2ccccc12)C1CC2CCC1C2 ; O=C(Oc1ccc([S+]2CCCC2)c2ccccc12)C1CC2CCC1C2 ; O=C(Oc1ccc([S+]2CCCCC2)c2ccccc12)C12CC3CC(CC(C3)C1)C2]

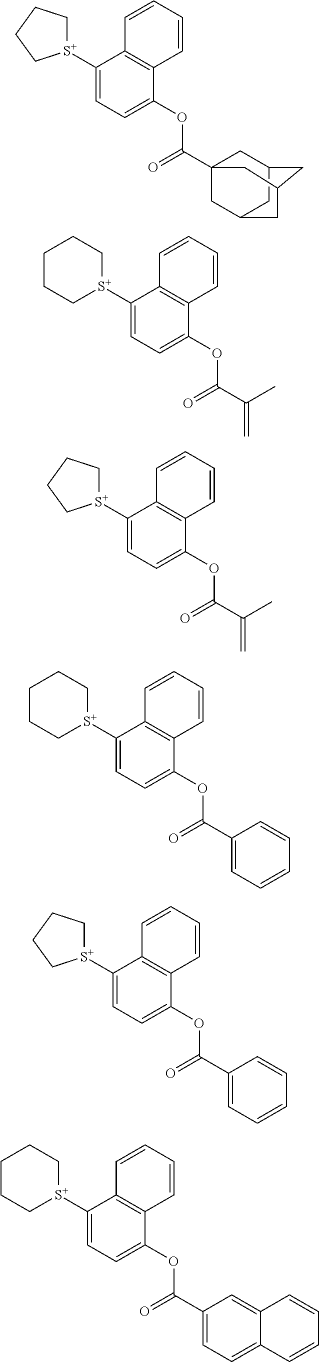 [SMILES: O=C(Oc1ccc([S+]2CCCC2)c2ccccc12)C12CC3CC(CC(C3)C1)C2 ; C=C(C)C(=O)Oc1ccc([S+]2CCCCC2)c2ccccc12 ; C=C(C)C(=O)Oc1ccc([S+]2CCCC2)c2ccccc12 ; O=C(Oc1ccc([S+]2CCCCC2)c2ccccc12)c1ccccc1 ; O=C(Oc1ccc([S+]2CCCC2)c2ccccc12)c1ccccc1 ; O=C(Oc1ccc([S+]2CCCCC2)c2ccccc12)c1ccc2ccccc2c1]

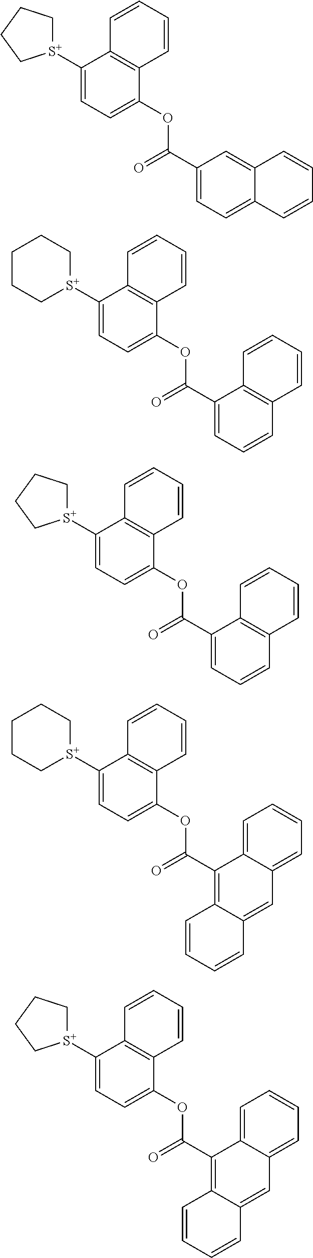 [SMILES: O=C(Oc1ccc([S+]2CCCC2)c2ccccc12)c1ccc2ccccc2c1 ; O=C(Oc1ccc([S+]2CCCCC2)c2ccccc12)c1cccc2ccccc12 ; O=C(Oc1ccc([S+]2CCCC2)c2ccccc12)c1cccc2ccccc12 ; O=C(Oc1ccc([S+]2CCCCC2)c2ccccc12)c1c2ccccc2cc2ccccc12 ; O=C(Oc1ccc([S+]2CCCC2)c2ccccc12)c1c2ccccc2cc2ccccc12]

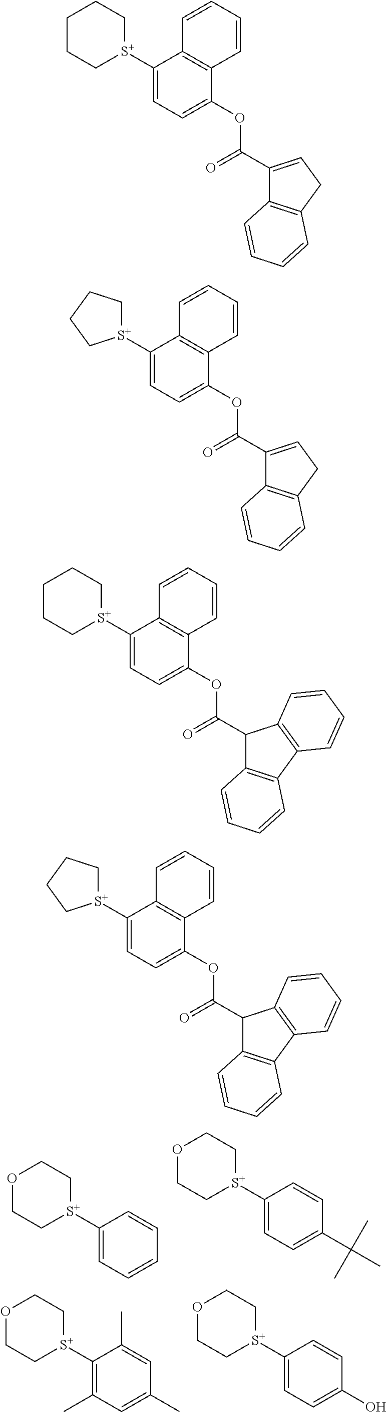

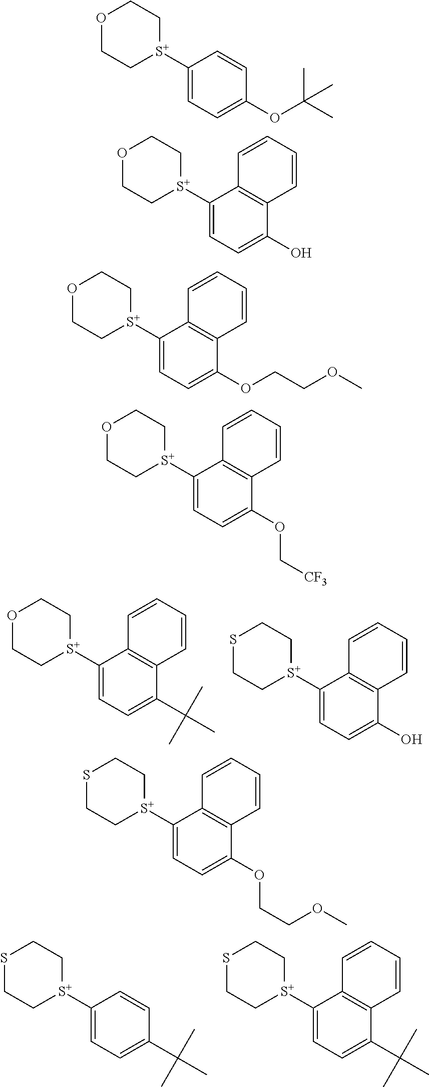

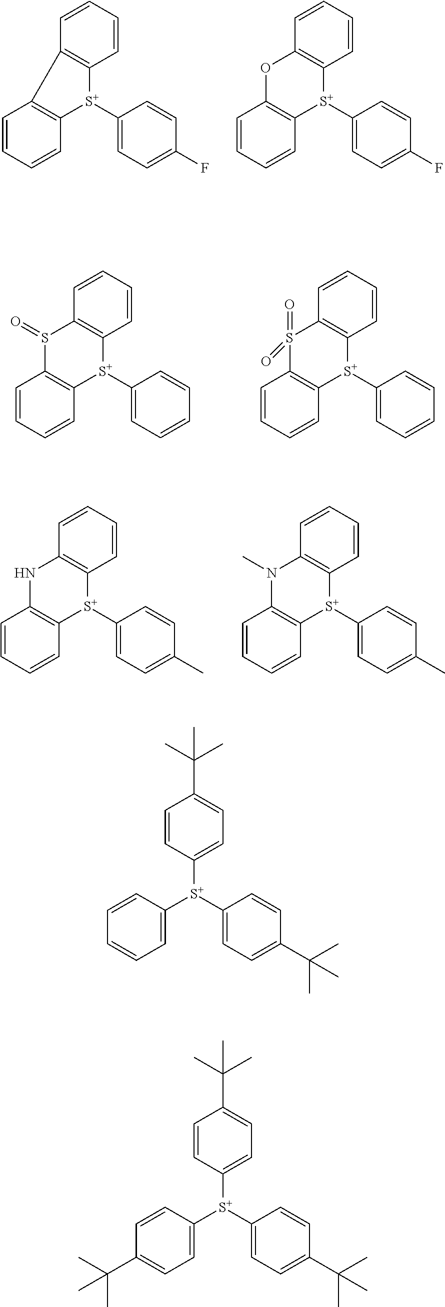

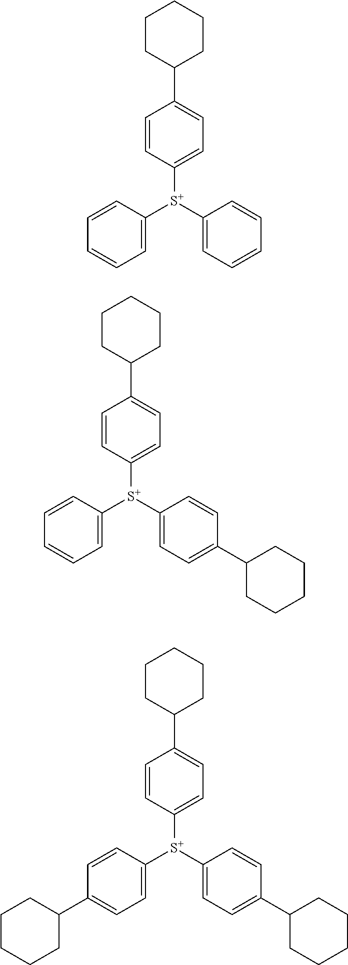 [SMILES: c1ccc([S+](c2ccccc2)c2ccc(C3CCCCC3)cc2)cc1 ; c1ccc([S+](c2ccc(C3CCCCC3)cc2)c2ccc(C3CCCCC3)cc2)cc1 ; c1cc([S+](c2ccc(C3CCCCC3)cc2)c2ccc(C3CCCCC3)cc2)ccc1C1CCCCC1]

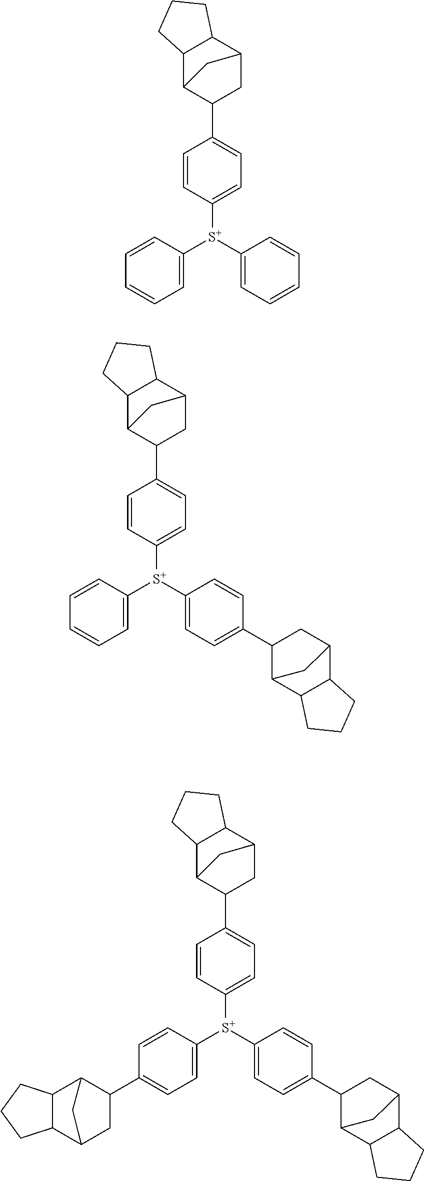 [SMILES: c1ccc([S+](c2ccccc2)c2ccc(C3CC4CC3C3CCCC43)cc2)cc1 ; c1ccc([S+](c2ccc(C3CC4CC3C3CCCC43)cc2)c2ccc(C3CC4CC3C3CCCC43)cc2)cc1 ; c1cc([S+](c2ccc(C3CC4CC3C3CCCC43)cc2)c2ccc(C3CC4CC3C3CCCC43)cc2)ccc1C1CC2CC1C1CCCC21]

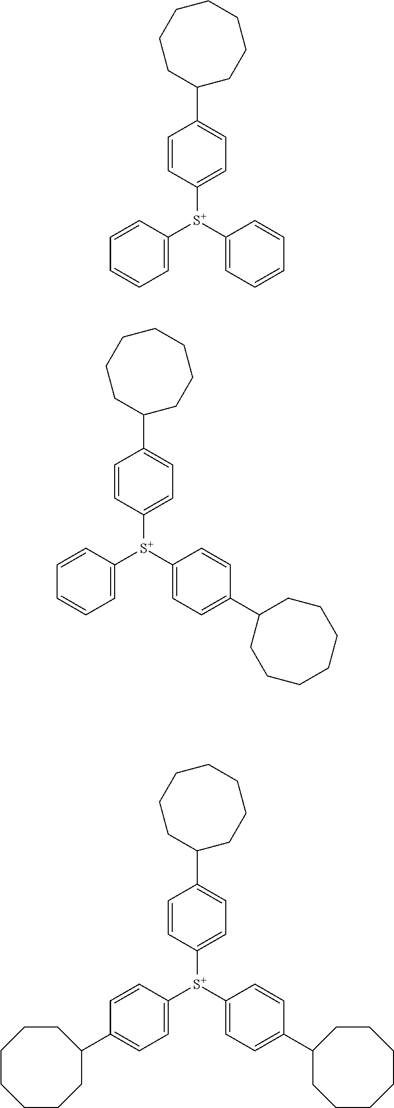 [SMILES: c1ccc([S+](c2ccccc2)c2ccc(C3CCCCCCC3)cc2)cc1 ; c1ccc([S+](c2ccc(C3CCCCCCC3)cc2)c2ccc(C3CCCCCCC3)cc2)cc1 ; c1cc([S+](c2ccc(C3CCCCCCC3)cc2)c2ccc(C3CCCCCCC3)cc2)ccc1C1CCCCCCC1]

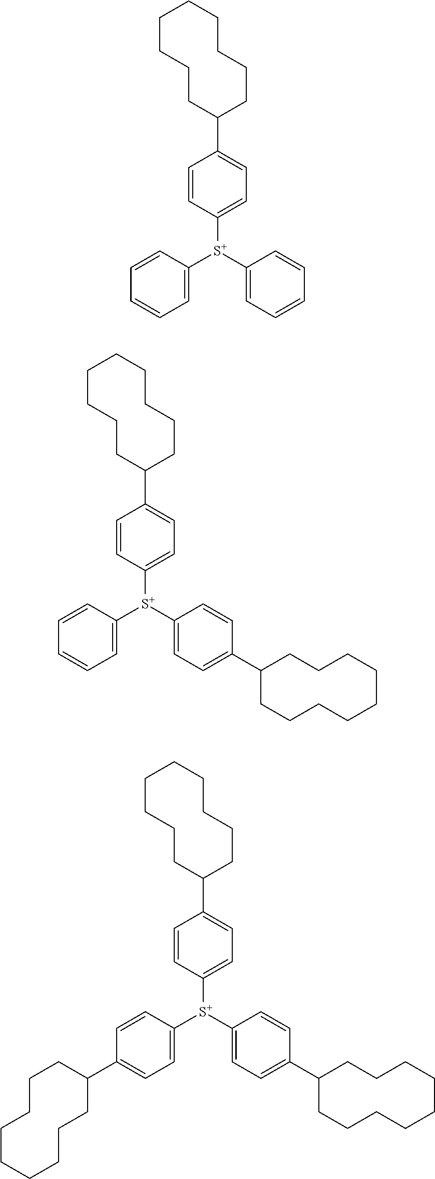 [SMILES: c1ccc([S+](c2ccccc2)c2ccc(C3CCCCCCCCC3)cc2)cc1 ; c1ccc([S+](c2ccc(C3CCCCCCCCC3)cc2)c2ccc(C3CCCCCCCCC3)cc2)cc1 ; c1cc([S+](c2ccc(C3CCCCCCCCC3)cc2)c2ccc(C3CCCCCCCCC3)cc2)ccc1C1CCCCCCCCC1]

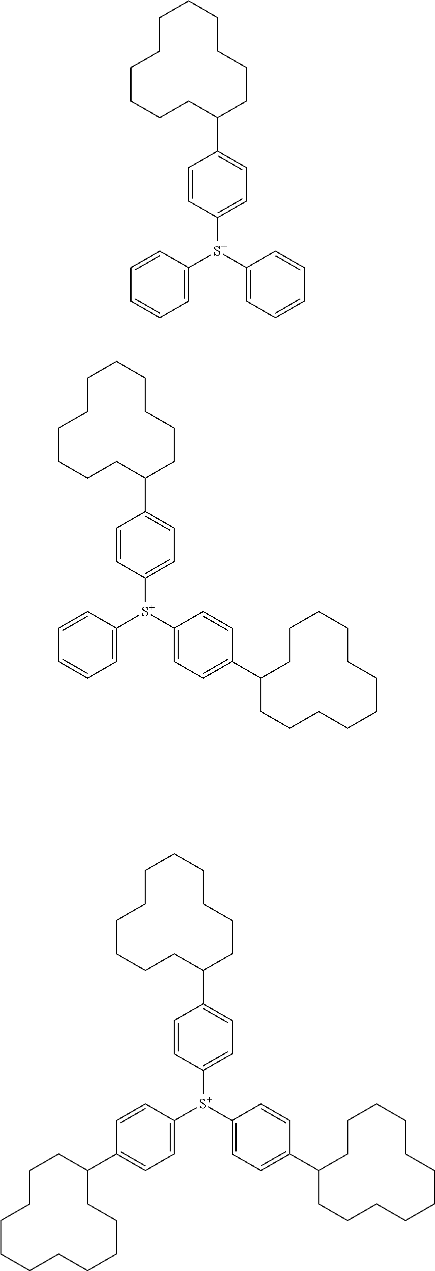 [SMILES: c1ccc([S+](c2ccccc2)c2ccc(C3CCCCCCCCCCC3)cc2)cc1 ; c1ccc([S+](c2ccc(C3CCCCCCCCCCC3)cc2)c2ccc(C3CCCCCCCCCCC3)cc2)cc1 ; c1cc([S+](c2ccc(C3CCCCCCCCCCC3)cc2)c2ccc(C3CCCCCCCCCCC3)cc2)ccc1C1CCCCCCCCCCC1]

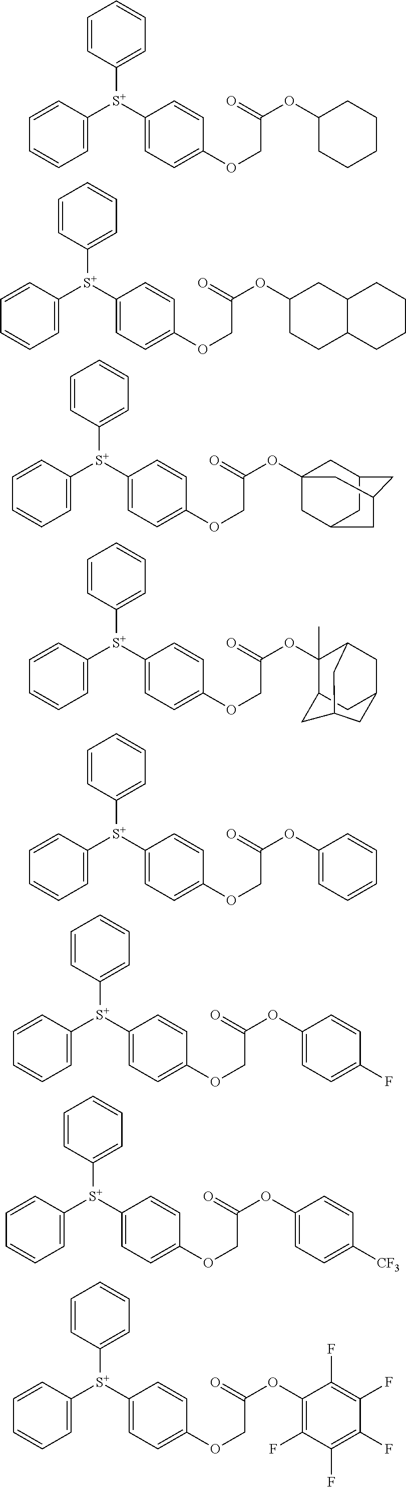

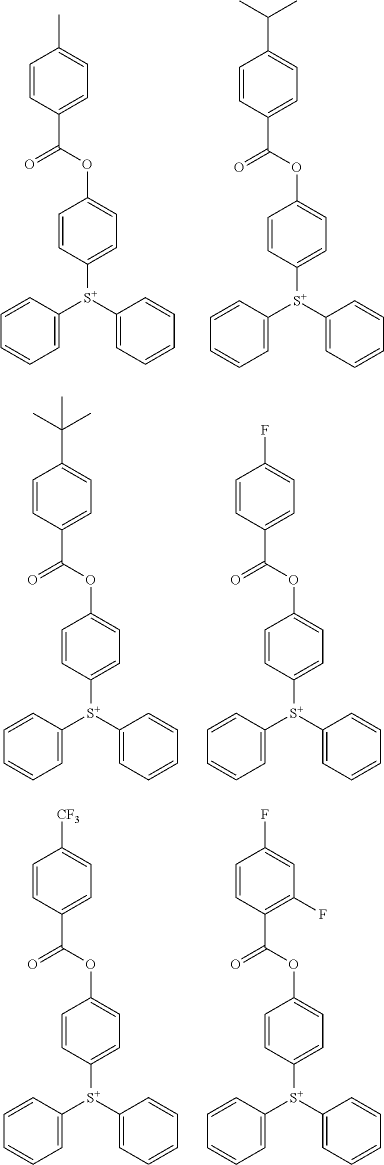 [SMILES: Cc1ccc(C(=O)Oc2ccc([S+](c3ccccc3)c3ccccc3)cc2)cc1 ; CC(C)c1ccc(C(=O)Oc2ccc([S+](c3ccccc3)c3ccccc3)cc2)cc1 ; CC(C)(C)c1ccc(C(=O)Oc2ccc([S+](c3ccccc3)c3ccccc3)cc2)cc1 ; O=C(Oc1ccc([S+](c2ccccc2)c2ccccc2)cc1)c1ccc(F)cc1 ; O=C(Oc1ccc([S+](c2ccccc2)c2ccccc2)cc1)c1ccc(C(F)(F)F)cc1 ; O=C(Oc1ccc([S+](c2ccccc2)c2ccccc2)cc1)c1ccc(F)cc1F]

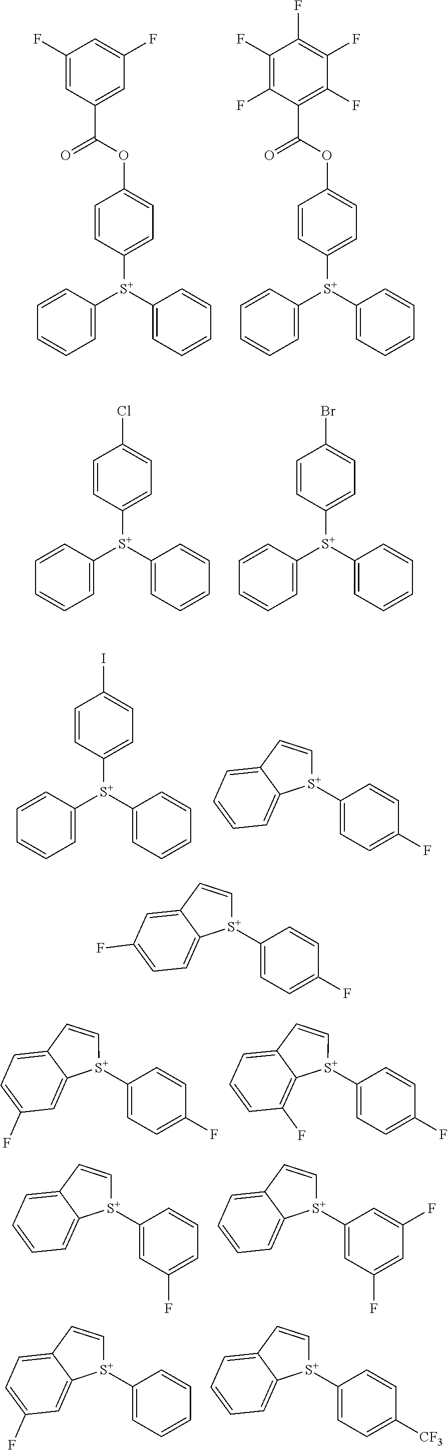

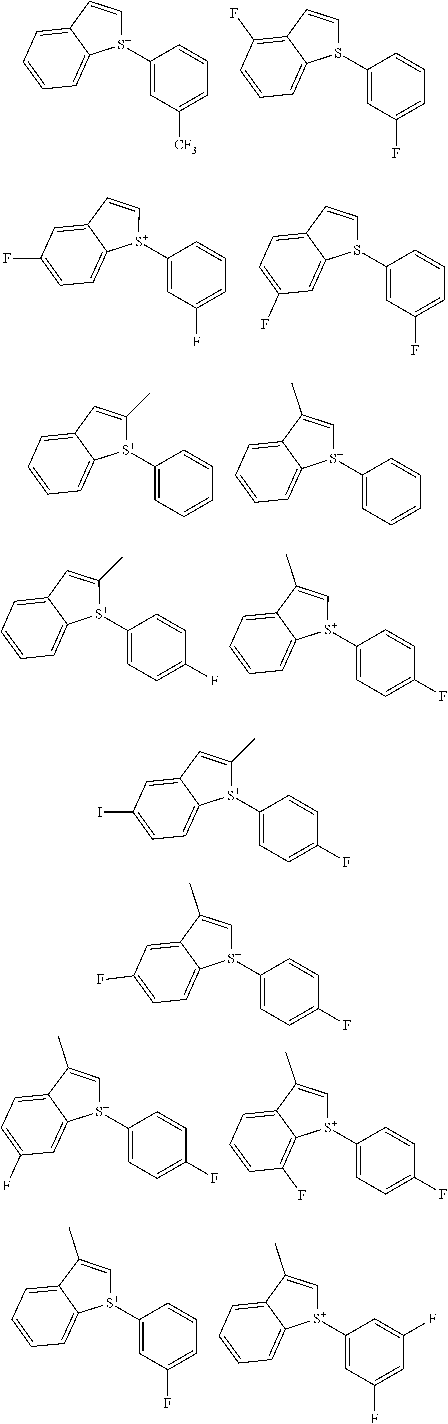

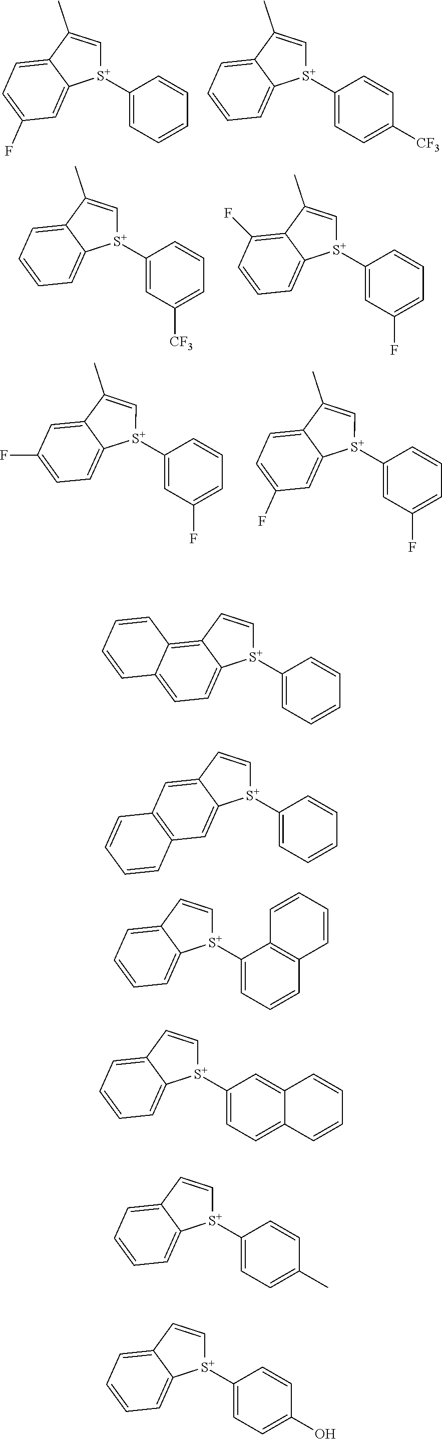

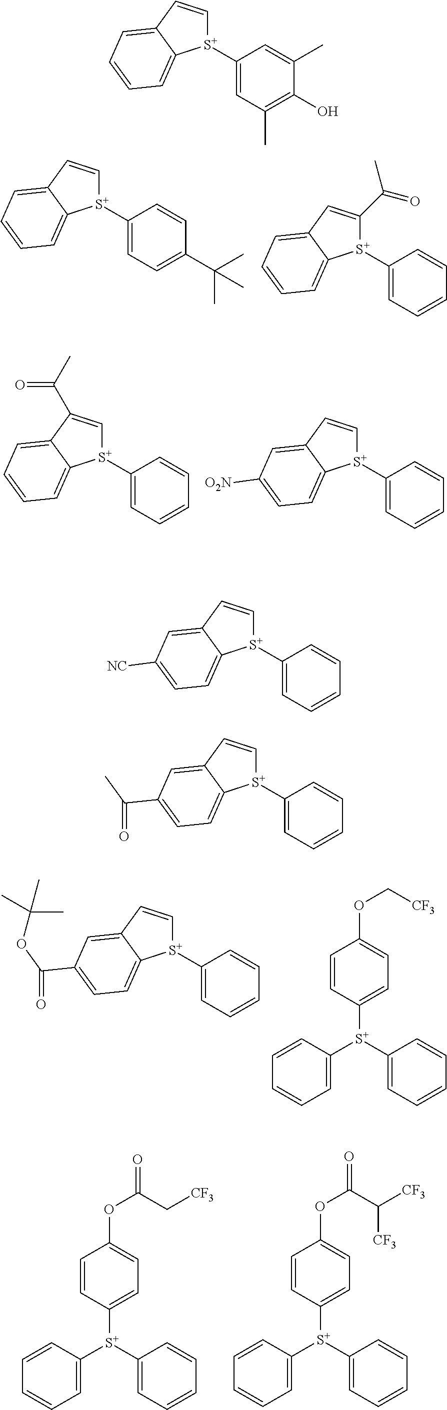

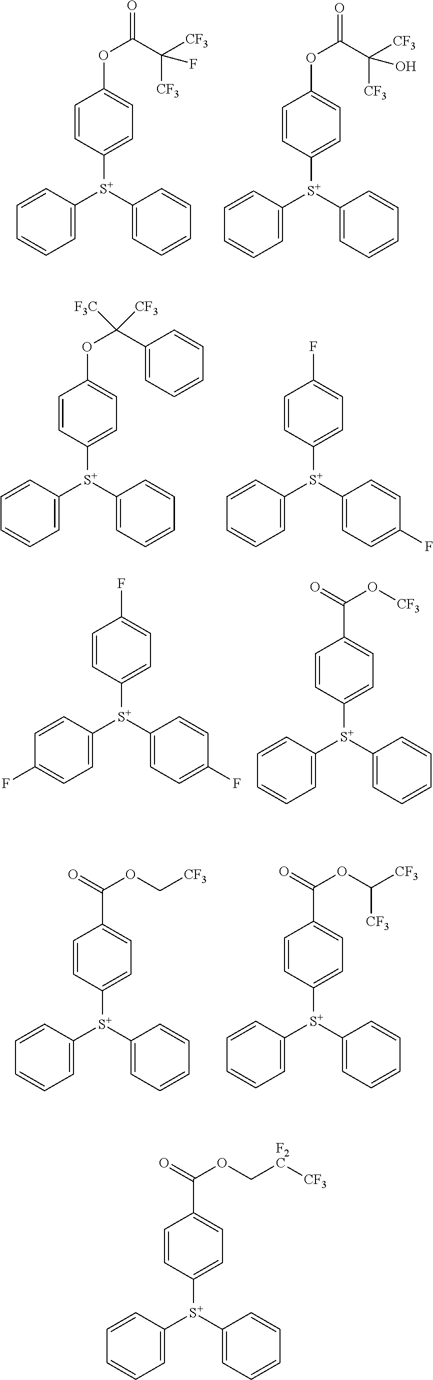

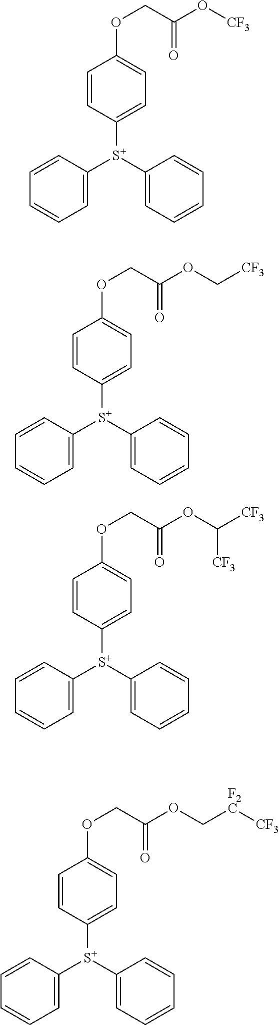 [SMILES: O=C(COc1ccc([S+](c2ccccc2)c2ccccc2)cc1)OC(F)(F)F ; O=C(COc1ccc([S+](c2ccccc2)c2ccccc2)cc1)OCC(F)(F)F ; O=C(COc1ccc([S+](c2ccccc2)c2ccccc2)cc1)OC(C(F)(F)F)C(F)(F)F ; O=C(COc1ccc([S+](c2ccccc2)c2ccccc2)cc1)OCC(F)(F)C(F)(F)F]

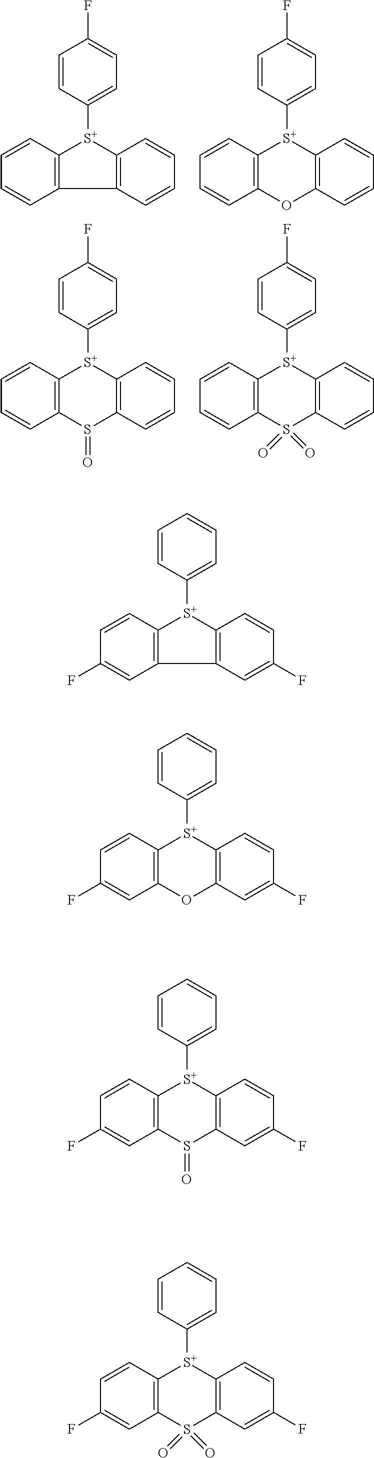

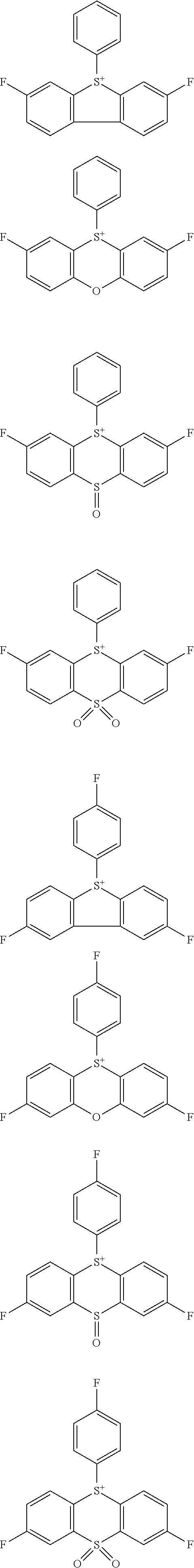

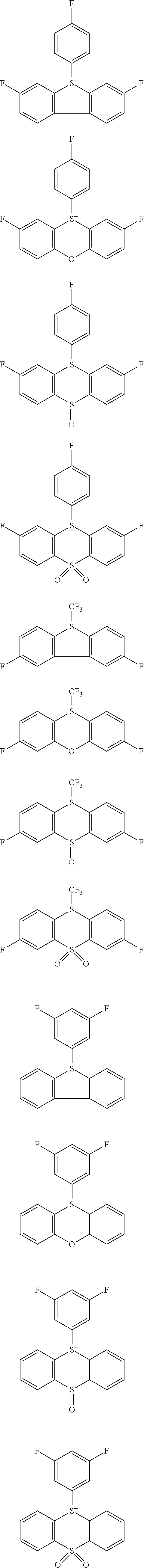

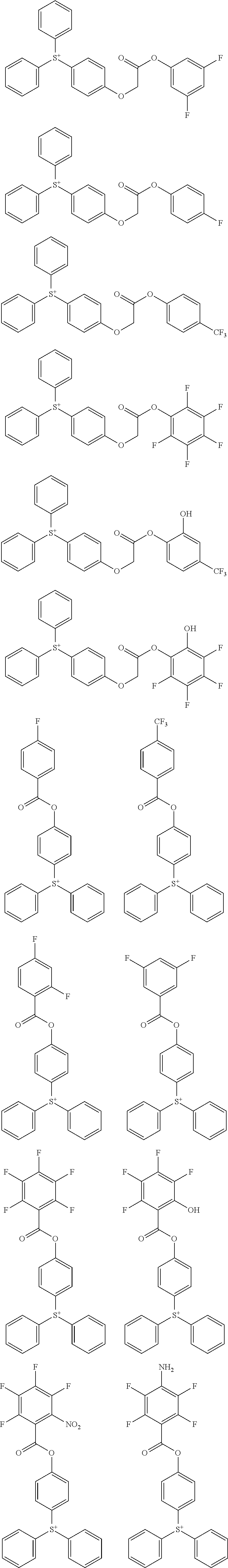

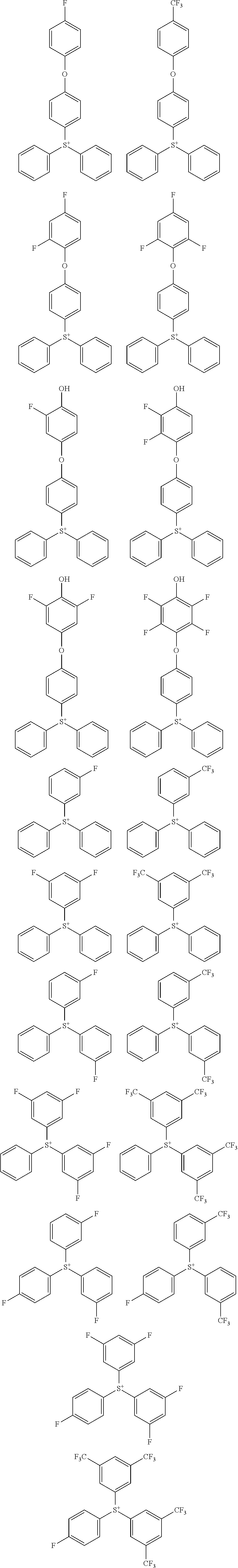

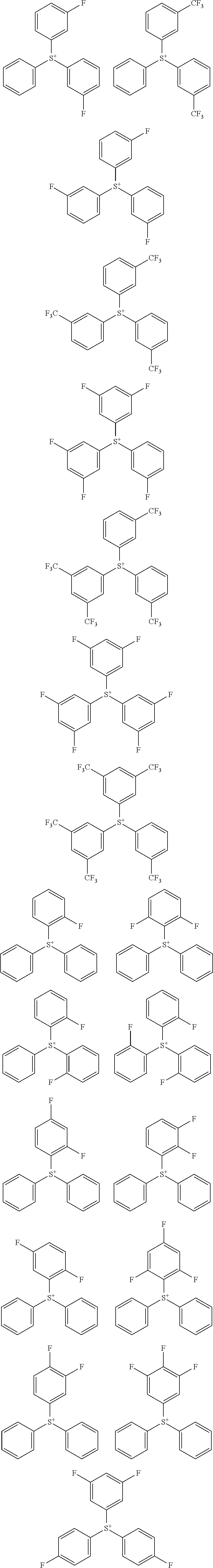

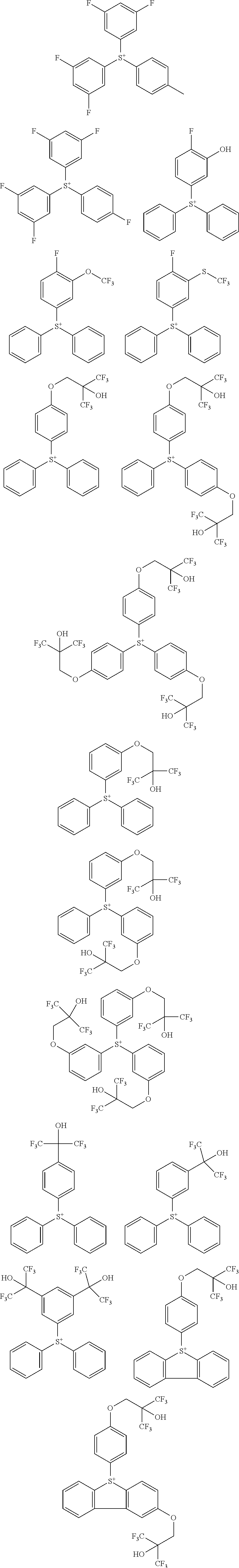

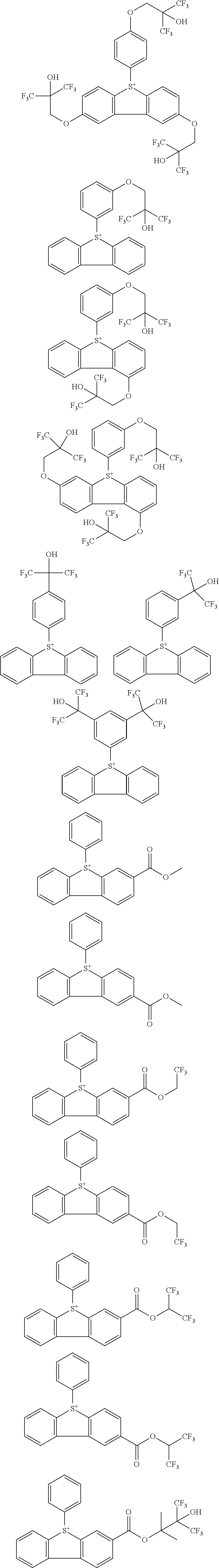

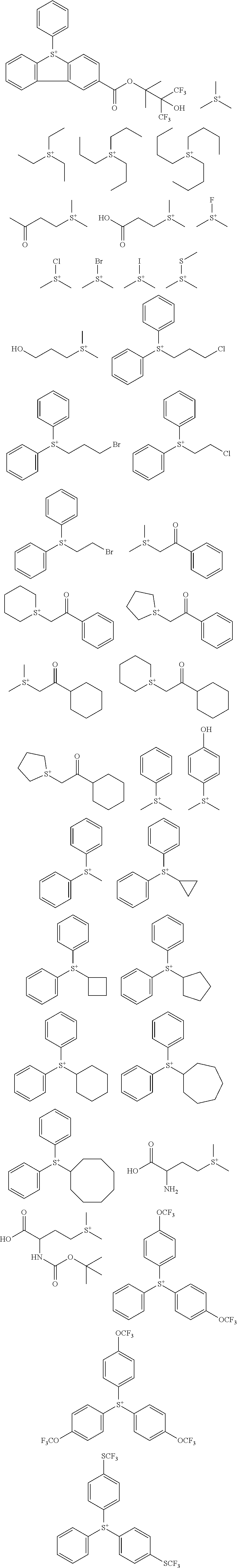

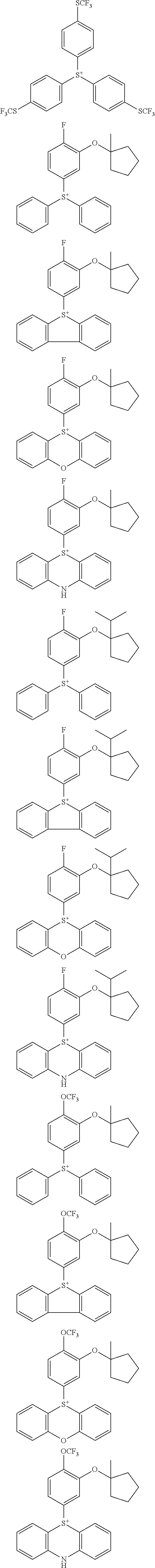

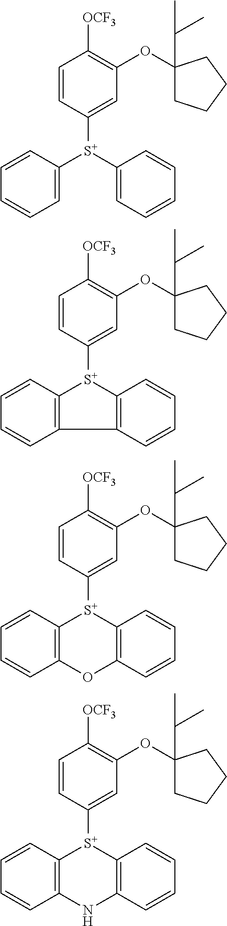 [SMILES: CC(C)C1(Oc2cc([S+](c3ccccc3)c3ccccc3)ccc2OC(F)(F)F)CCCC1 ; CC(C)C1(Oc2cc(-[s+]3c4ccccc4c4ccccc43)ccc2OC(F)(F)F)CCCC1 ; CC(C)C1(Oc2cc([S+]3c4ccccc4Oc4ccccc43)ccc2OC(F)(F)F)CCCC1 ; CC(C)C1(Oc2cc([S+]3c4ccccc4Nc4ccccc43)ccc2OC(F)(F)F)CCCC1]

Specific examples of the iodonium cation having formula (Z-2) include, but are not limited to, those listed below.

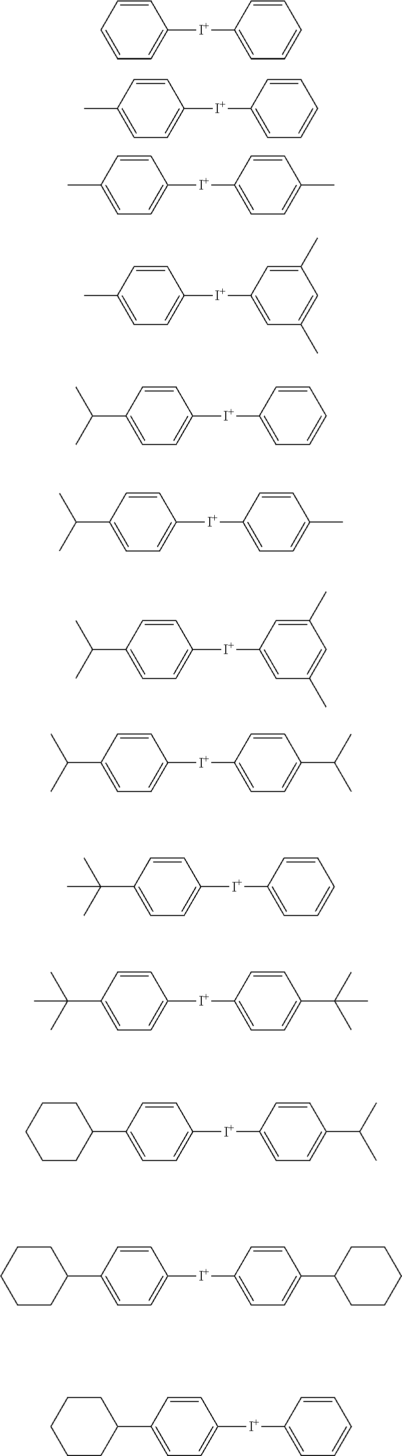

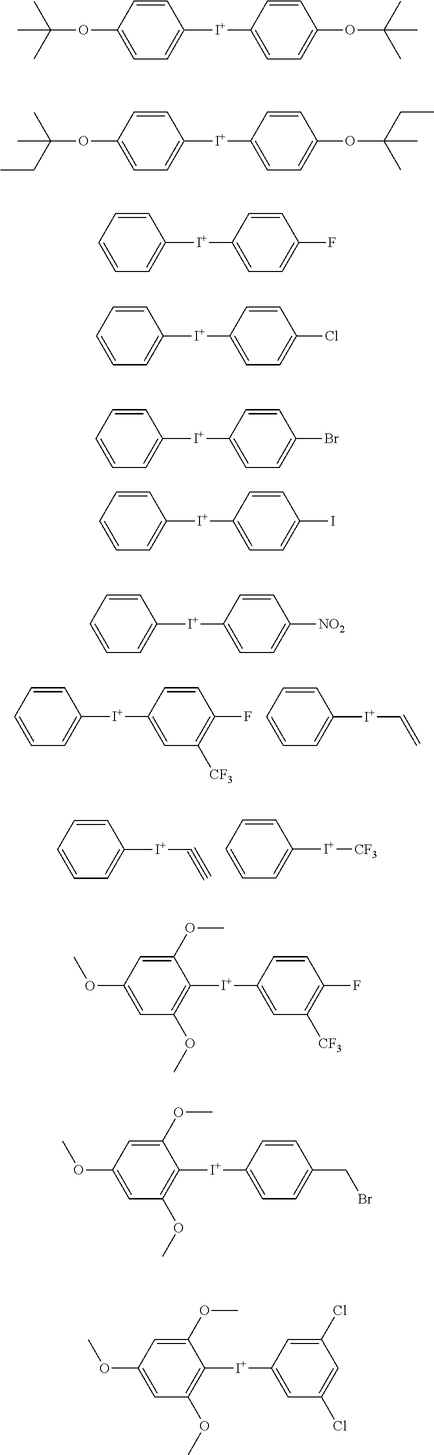

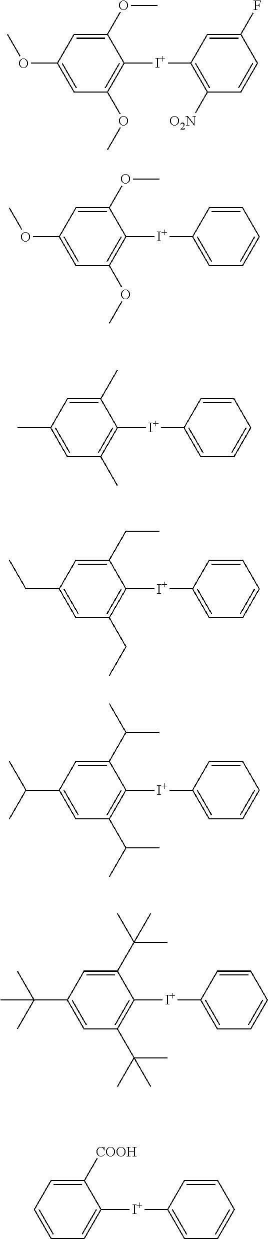

The onium cation of Z⁺ is preferably a sulfonium cation having the following formula (Z-3):

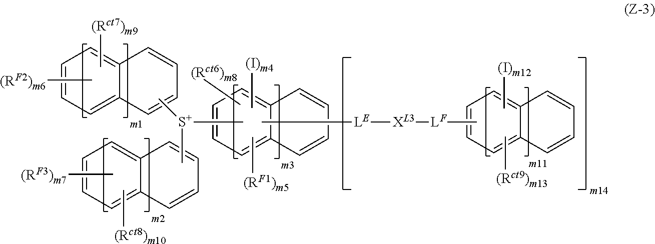

In formula (Z-3), m1 is 0 or 1. When m1 is 0, it is a benzene ring, and when m1 is 1, it is a naphthalene ring, but a benzene ring in which m1 is 0 is preferable from a viewpoint of solvent solubility. m2 is 0 or 1. When m2 is 0, it is a benzene ring, and when m2 is 1, it is a naphthalene ring, but a benzene ring in which m2 is 0 is preferable from a viewpoint of solvent solubility. m3 is 0 or 1. When m3 is 0, it is a benzene ring, and when m3 is 1, it is a naphthalene ring, but a benzene ring in which m3 is 0 is preferable from a viewpoint of solvent solubility.

In formula (Z-3), m4 is 0, 1, 2, 3, or 4. The larger the number of iodine atoms in the cation structure, the higher absorption particularly for EUV, but since there is a concern that solvent solubility is poor and precipitation occurs in a resist composition, m4 is preferably 0, 1, 2, or 3, and more preferably 0, 1, or 2.

In formula (Z-3), m5 is 0, 1, 2, 3, or 4. m5 is preferably 0, 1, 2, or 3, and more preferably 0, 1, or 2 from a viewpoint of raw material procurement. m6 is 0, 1, 2, 3, 4, 5, or 6. m6 is preferably 0, 1, 2, or 3, and more preferably 0, 1, or 2 from a viewpoint of raw material procurement. m7 is 0, 1, 2, 3, 4, 5, or 6. m7 is preferably 0, 1, 2, or 3, and more preferably 0, 1, or 2 from a viewpoint of raw material procurement.

In formula (Z-3), m8 is 0, 1, or 2. m8 is preferably 0 or 1 from a viewpoint of raw material procurement. m9 is 0, 1, or 2. m9 is preferably 0 or 1 from a viewpoint of raw material procurement. m10 is 0, 1, or 2. m10 is preferably 0 or 1 from a viewpoint of raw material procurement.

In formula (Z-3), m11 is 0 or 1. When m11 is 0, it is a benzene ring, and when m11 is 1, it is a naphthalene ring, but a benzene ring in which m11 is 0 is preferable from a viewpoint of solvent solubility.

In formula (Z-3), m12 is 0, 1, 2, 3, or 4. The larger the number of iodine atoms in the cation structure, the higher absorption particularly for EUV, but since there is a concern that solvent solubility is poor and precipitation occurs in a resist composition, m12 is preferably 0, 1, 2, or 3, and more preferably 0, 1, or 2.

In formula (Z-3), m13 is 0, 1, or 2. m13 is preferably 0 or 1 from a viewpoint of raw material procurement. m14 is 0, 1, or 2. m14 is preferably 0 or 1 from a viewpoint of synthesis.

Provided that 0≤m6+m9≤4 when m1 is 0, and 0≤m6+m9≤6 when m1 is 1. 0≤m7+m10≤4 when m2 is 0, and 0≤m7+m10≤6 when m2 is 1. 1≤m4+m5+m8+m14≤4 when m3 is 0, and 1≤m4+m5+m8+m14≤6 when m3 is 1. 0≤m12+m13≤4 when m11 is 0, and 0≤m12+m13≤6 when m11 is 1. m4+m12≥1.

In formula (Z-3), R^{F1} to R^{F3} are each independently a fluorine atom, a C₁-C₆ fluorinated saturated hydrocarbyl group, a C₁-C₆ fluorinated saturated hydrocarbyloxy group, or a C₁-C₆ fluorinated saturated hydrocarbylthio group. Among these, R^{F1} to R^{F3} are each preferably a trifluoromethyl group, a trifluoromethoxy group, or a trifluorothiomethoxy group. When m5 is 2 or more, R^{F1}s may be the same as or different from each other, when m6 is 2 or more, R^{F2}s may be the same as or different from each other, and when m7 is 2 or more, R^{F3}s may be the same as or different from each other.

In formula (Z-3), each of R^{ct6} to R^{ct9} is a halogen atom other than an iodine atom and a fluorine atom, a nitro group, a cyano group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. The hydrocarbyl group and hydrocarbyl moieties of the hydrocarbyloxy group and the hydrocarbylthio group may be saturated or unsaturated, and may be linear, branched, or cyclic. Specific examples thereof include those similar to those exemplified as the hydrocarbyl group of R¹ in the description of formula (a). Some or all of hydrogen atoms of the hydrocarbyl group and hydrocarbyl moieties of the hydrocarbyloxy group and the hydrocarbylthio group may be replaced with a group containing a heteroatom such as an oxygen atom, a sulfur atom, a nitrogen atom, or a halogen atom, some of (—CH₂—)s in the hydrocarbyl group may be replaced with a group containing a heteroatom such as an oxygen atom, a sulfur atom, or a nitrogen atom, and as a result, the hydrocarbyl group may contain a hydroxy group, a cyano group, a fluorine atom, a chlorine atom, a bromine atom, an iodine atom, a carbonyl group, an ether bond, an ester bond, a sulfonic acid ester bond, a carbonate bond, a lactone ring, a sultone ring, a carboxylic acid anhydride (—C(═O)—O—C(═O)—), a haloalkyl group, or the like.

When m8 is 2, two R^{ct6}s may be the same or different from each other, and may be bonded to each other to form a ring together with carbon atoms to which they are bonded, when m9 is 2, two R^{ct7}s may be the same or different from each other, and may be bonded to each other to form a ring together with carbon atoms to which they are bonded, when m10 is 2, two R^{ct8}s may be the same or different from each other, and may be bonded to each other to form a ring together with carbon atoms to which they are bonded, and when m13 is 2, two R^{ct9}s may be the same or different from each other, and may be bonded to each other to form a ring with carbon atoms to which they are bonded. Specific examples of the ring formed at this time include a cyclopropane ring, a cyclobutane ring, a cyclopentane ring, a cyclohexane ring, a norbornane ring, and an adamantane ring. Some or all of hydrogen atoms of the ring may be replaced with a group containing a heteroatom such as an oxygen atom, a sulfur atom, a nitrogen atom, or a halogen atom, some of (—CH₂—)s in the ring may be replaced with a group containing a heteroatom such as an oxygen atom, a sulfur atom, or a nitrogen atom, and as a result, the ring may contain a hydroxy group, a fluorine atom, a chlorine atom, a bromine atom, an iodine atom, a cyano group, a carbonyl group, an ether bond, an ester bond, a sulfonic acid ester bond, a carbonate bond, a lactone ring, a sultone ring, a carboxylic acid anhydride (—C(═O)—O—C(═O)—), a haloalkyl group, or the like.

Aromatic rings directly bonded to S⁺ in the sulfonium cation having formula (Z-3) may be bonded to each other to form a ring together with S+. At this time, specific examples of the structure of the ring include those having the following formulas:

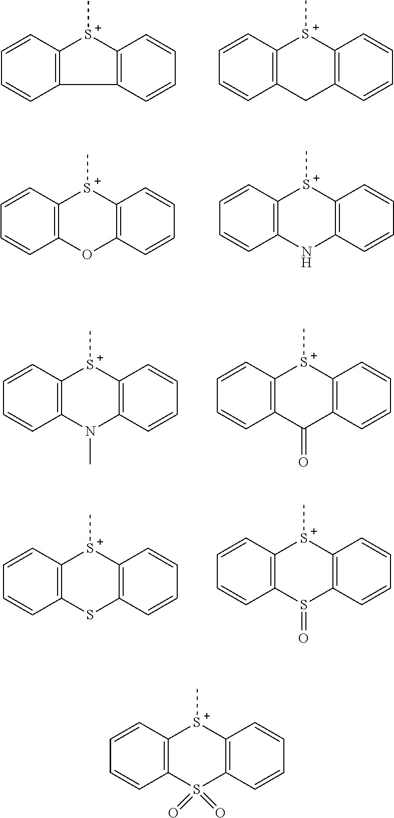

wherein the dashed line is a bond.

In formula (Z-3), L^{E} and L^{F} are each independently a single bond, an ether bond, an ester bond, an amide bond, a sulfonic acid ester bond, a sulfonic acid amide bond, a carbonate bond, or a carbamate bond. Among these, L^{E} is preferably a single bond, an ether bond, an ester bond, or a sulfonic acid ester bond, and more preferably an ester bond or a sulfonic acid ester bond. L^{F} is preferably a single bond, an ether bond, or an ester bond, and more preferably a single bond.

In formula (Z-3), X^{L3} is a single bond or a C₁-C₄₀ hydrocarbylene group which may contain a heteroatom. Specific examples of the C₁-C₄₀ hydrocarbylene group which may contain a heteroatom include, but are not limited to, those similar to those exemplified as specific examples of the C₁-C₄₀ hydrocarbylene group which may contain a heteroatom, represented by X^{L1} and X^{L2}.

The sulfonium cation having formula (Z-3) preferably has the following formula (Z-3-1):

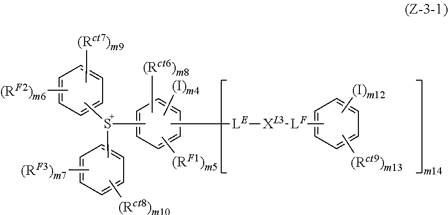

wherein m4 to m10, m12 to m14, R^{F1} to R^{F3}, R^{ct6} to R^{ct9}, L^{E}, L^{F}, and X^{L3} are as defined above.

The cation having formula (Z-3-1) preferably has the following formula (Z-3-2):

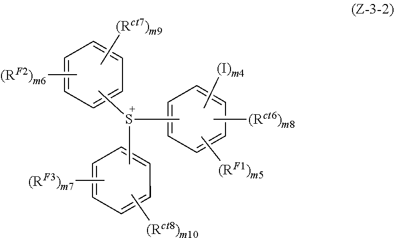

wherein m4 to m10, R^{F1} to R^{F3}, and R^{ct6} to Rct8 are as defined above.

Specific examples of the sulfonium cation having formula (Z-3) include, but are not limited to, those listed below. Note that, in the following formulas, Me is a methyl group.

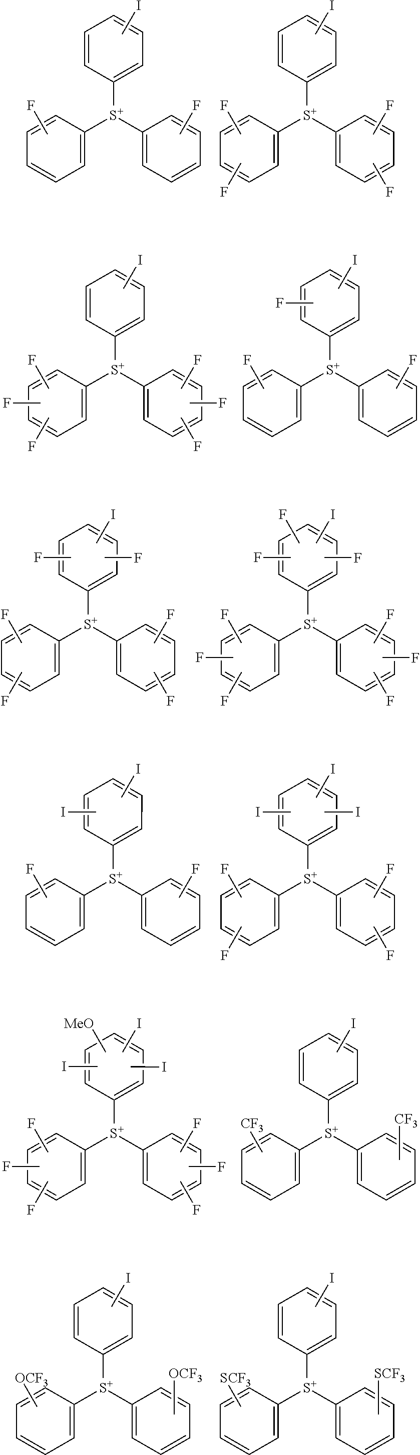

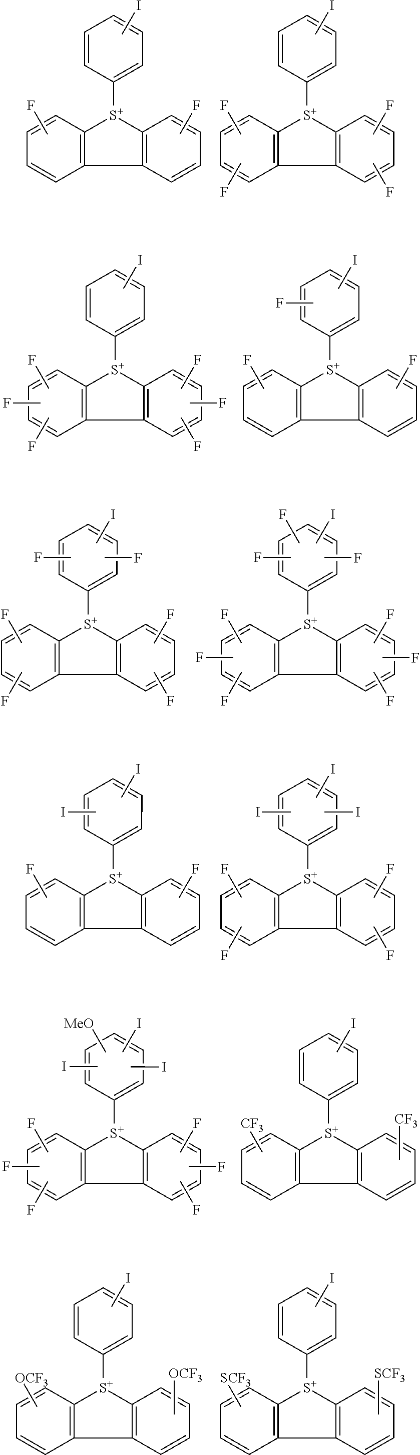

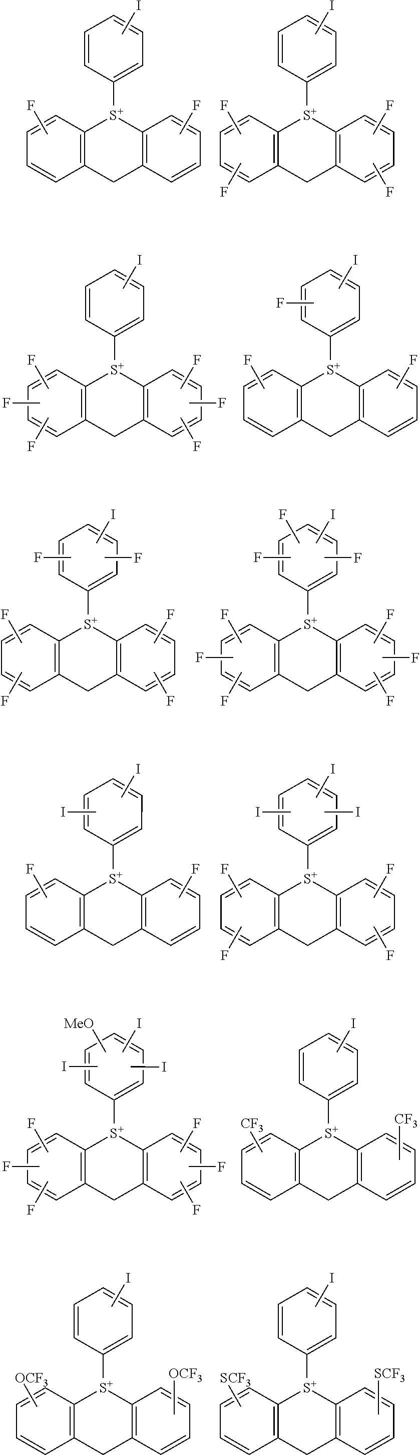

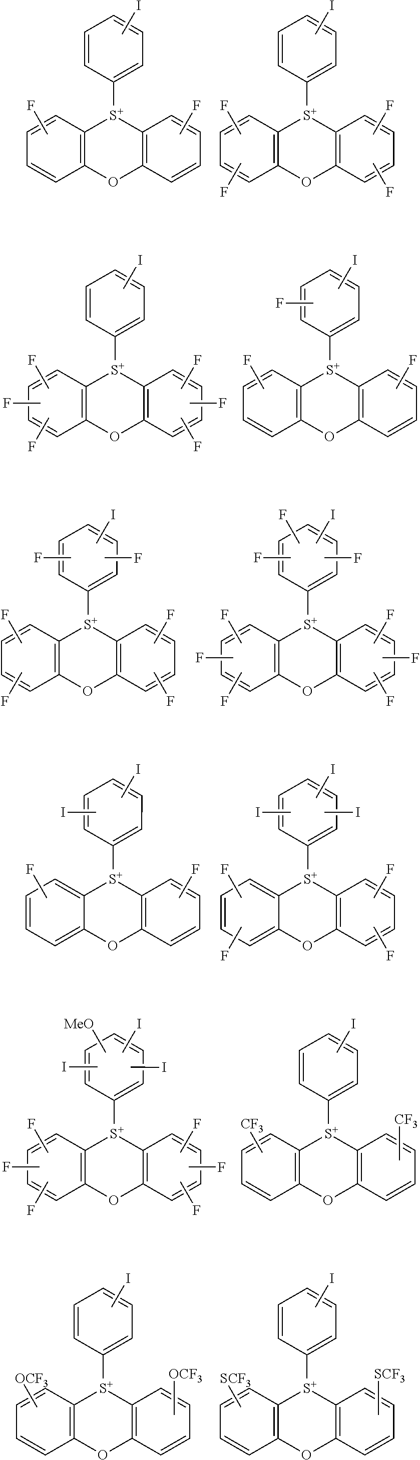

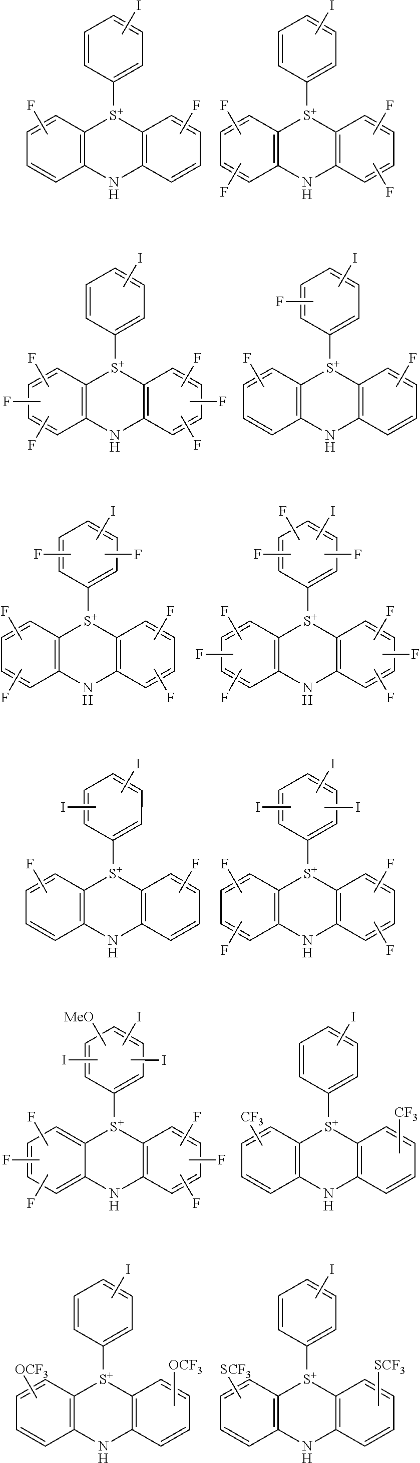

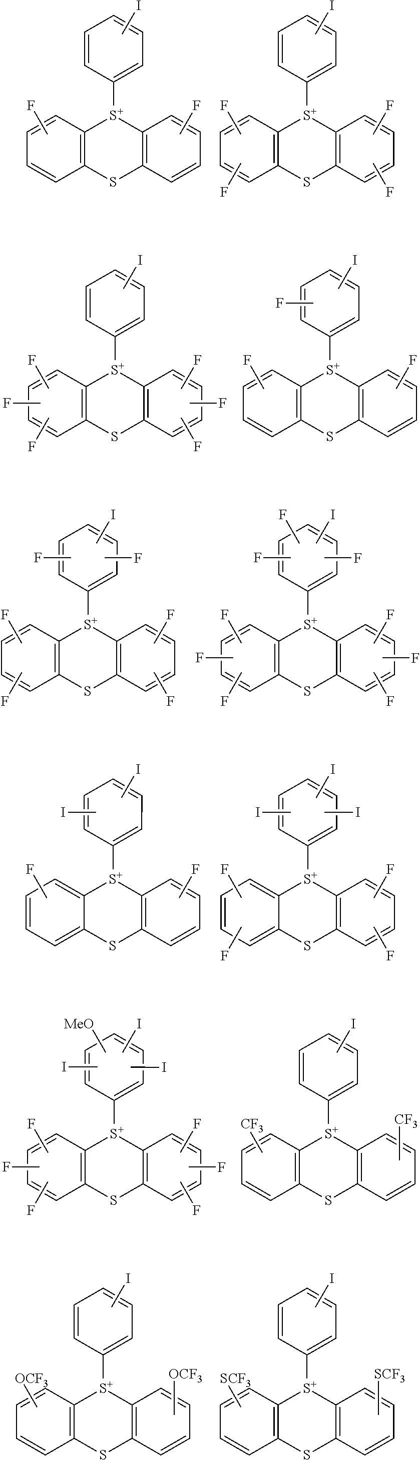

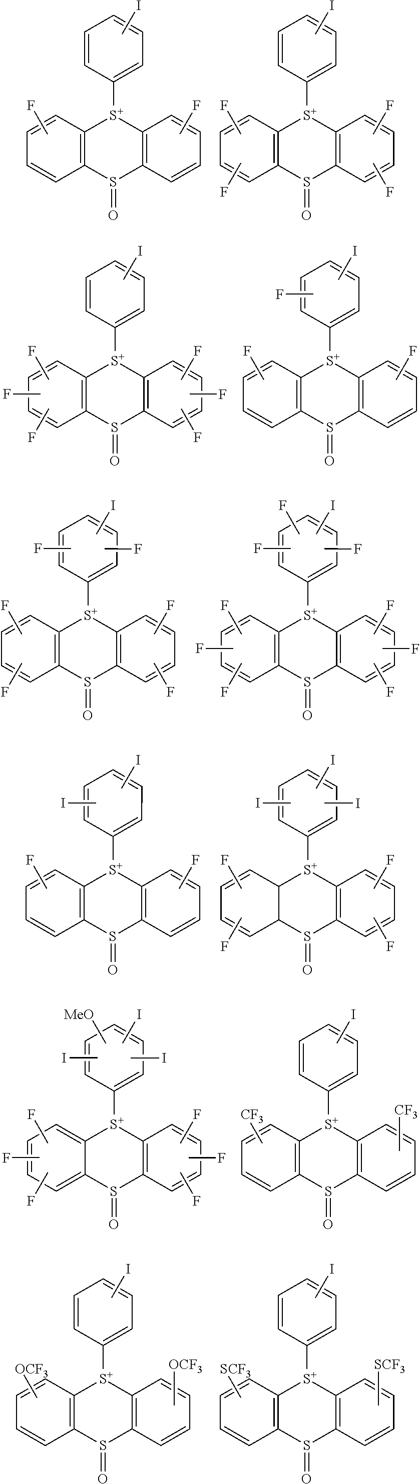

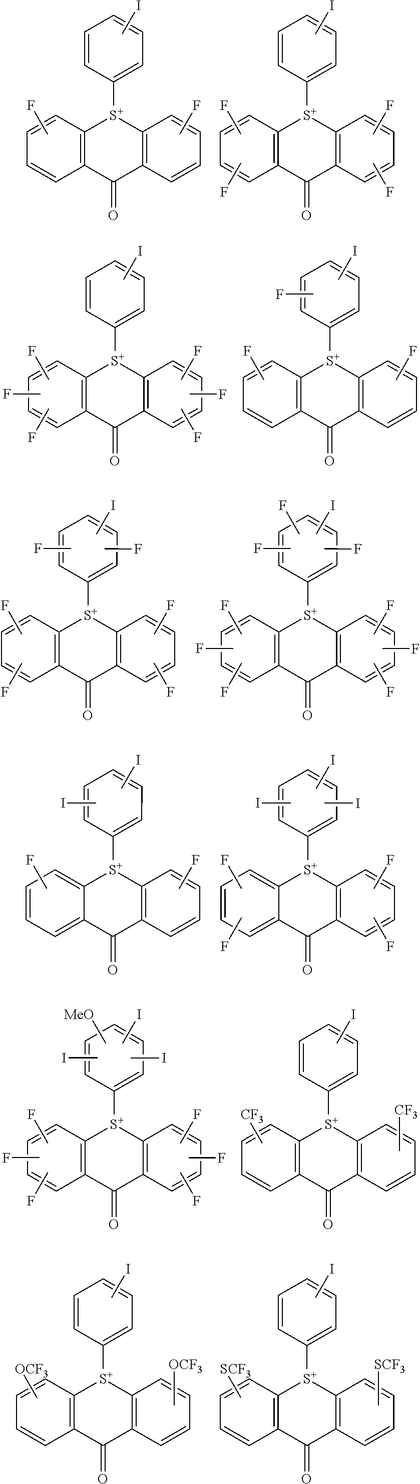

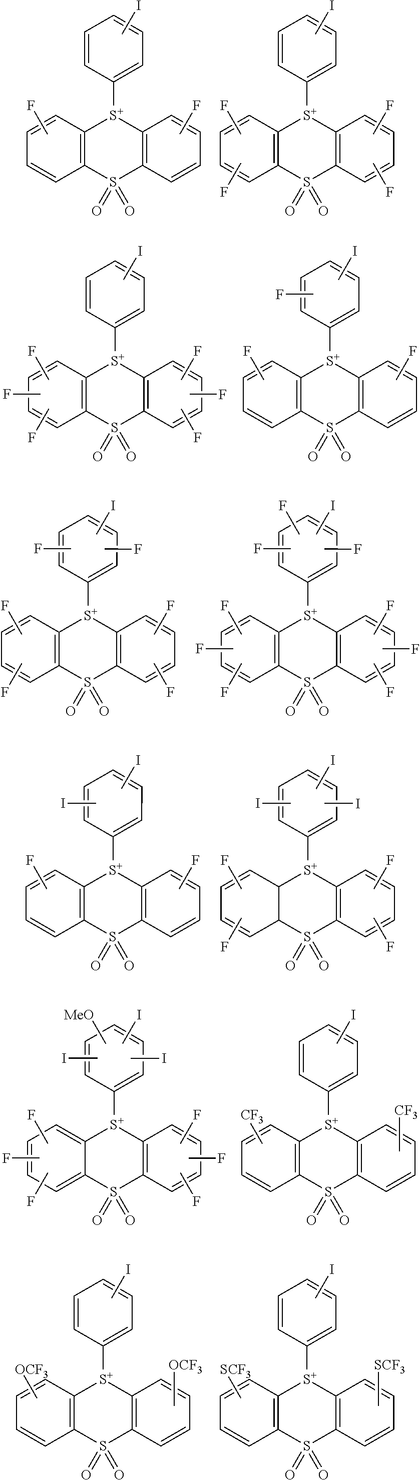

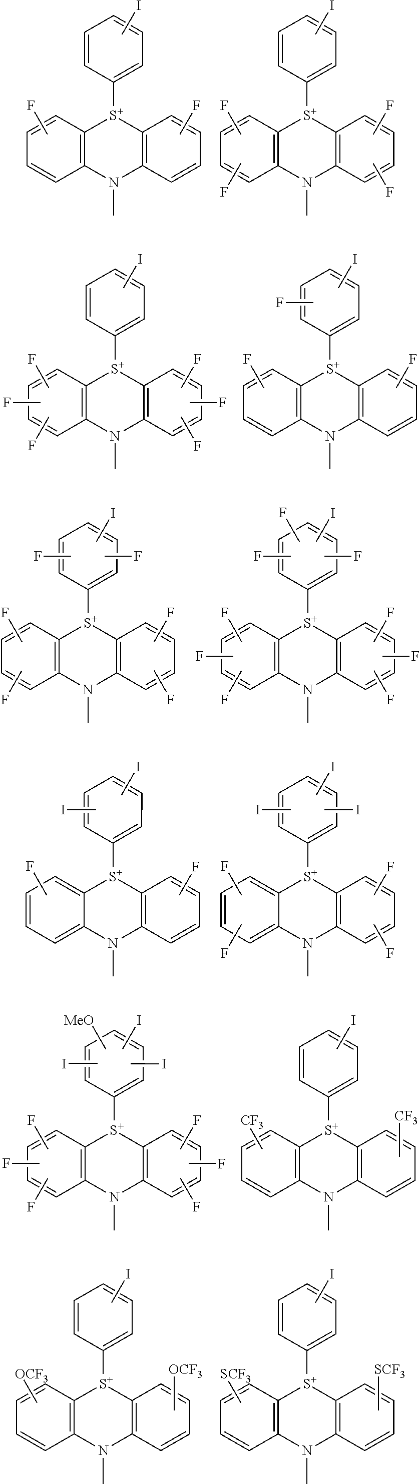

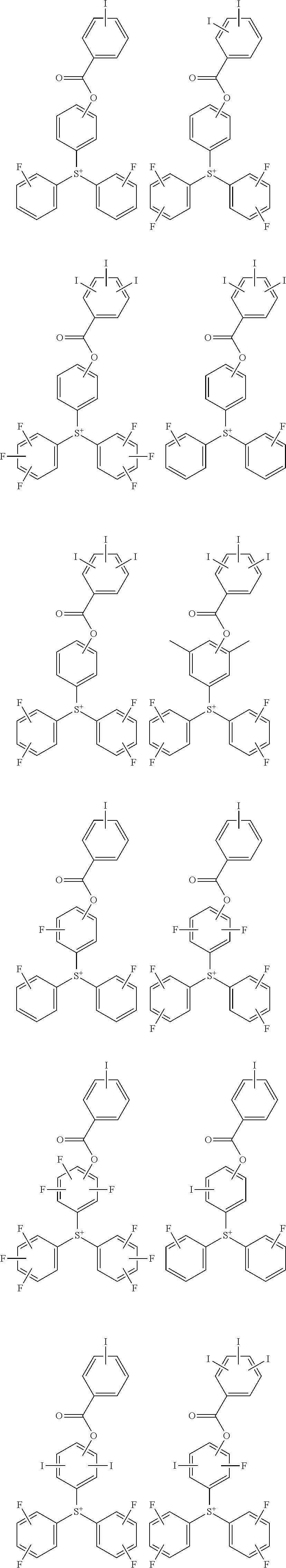

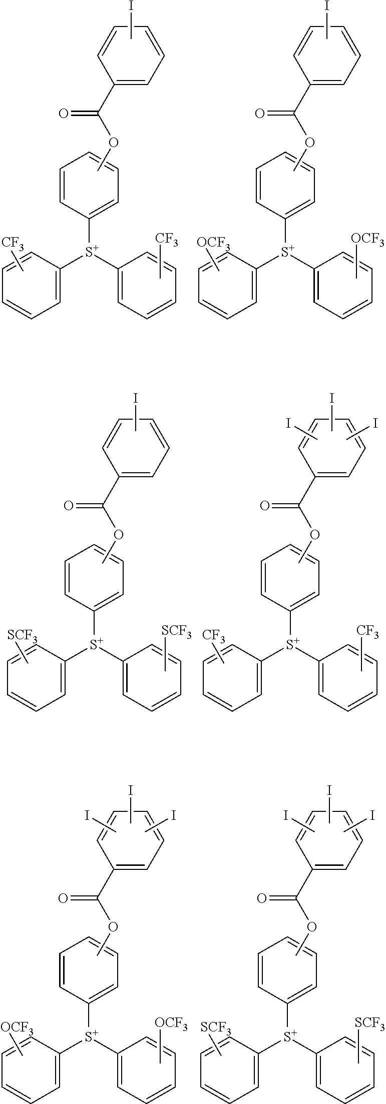

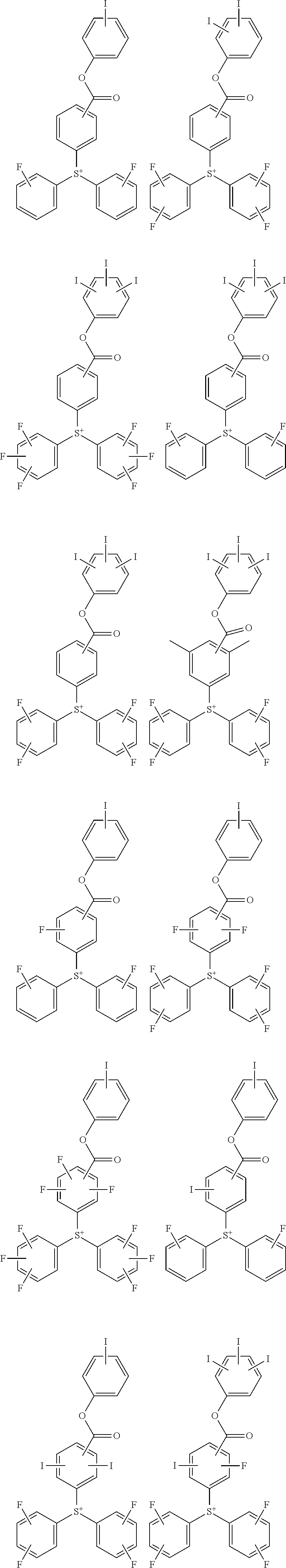

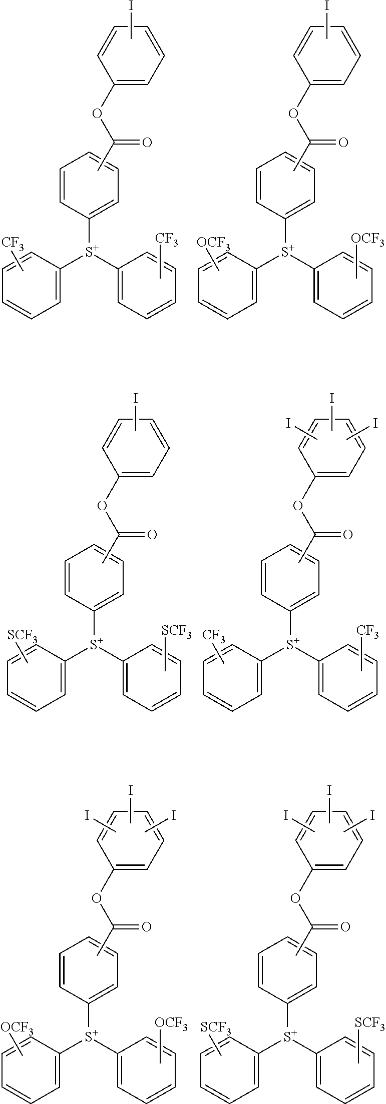

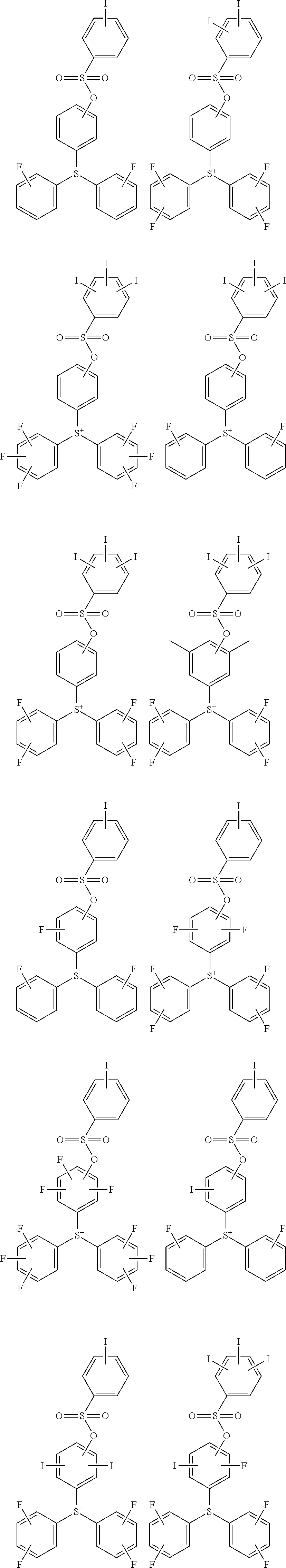

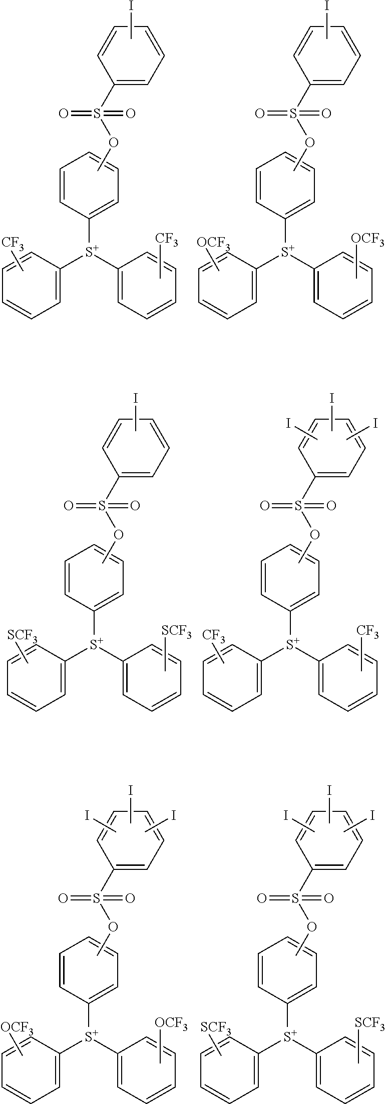

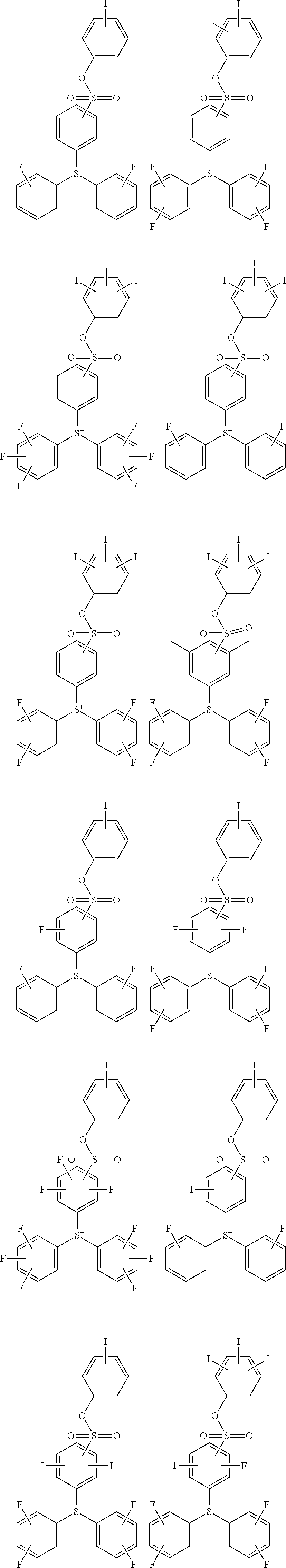

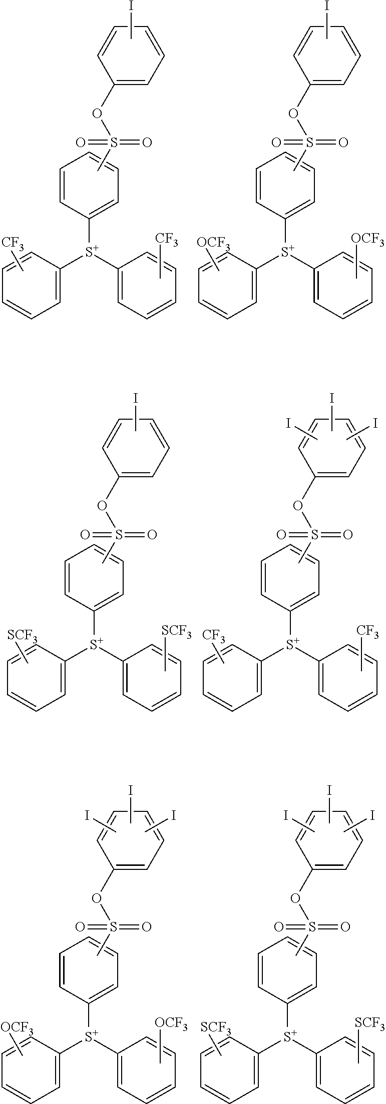

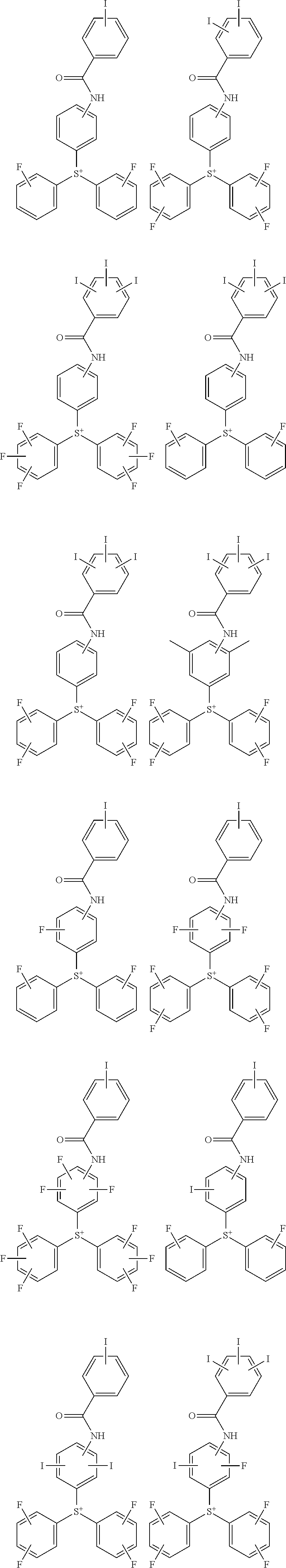

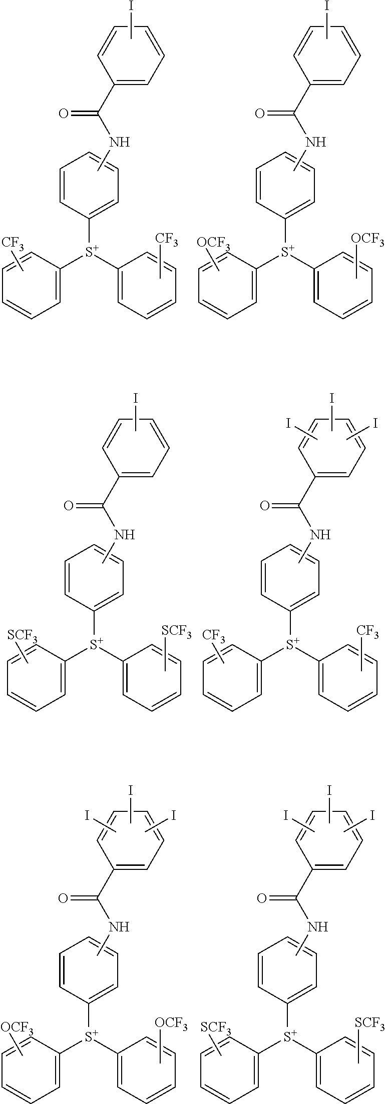

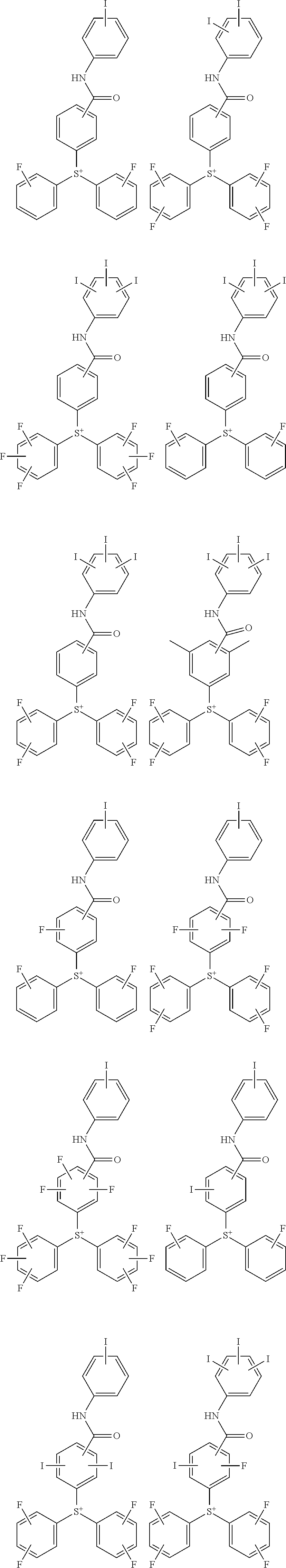

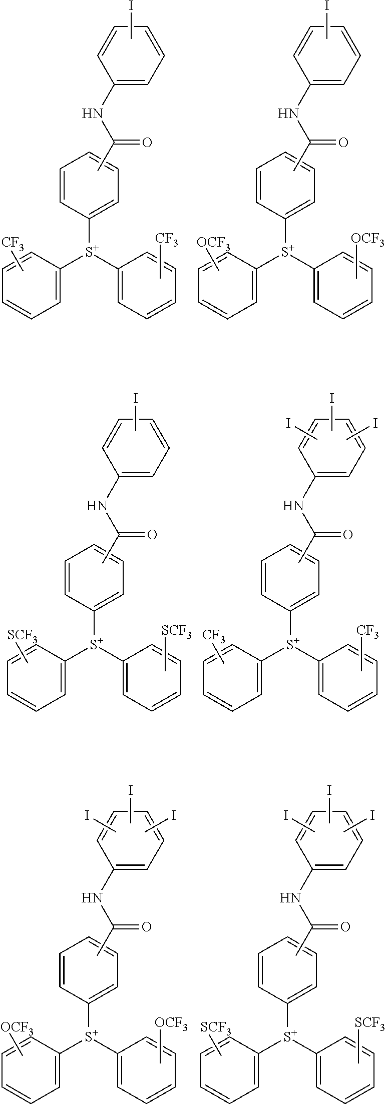

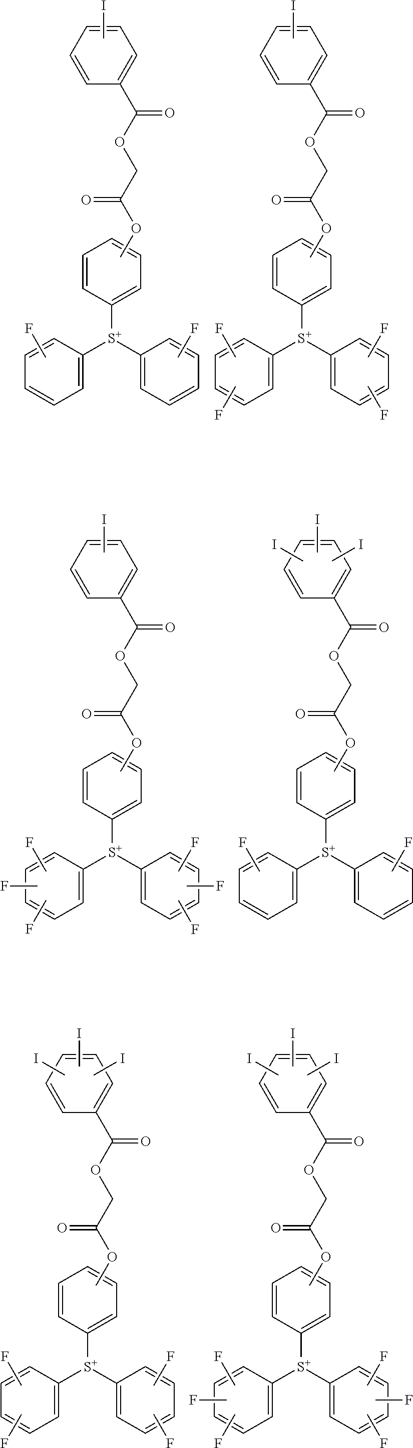

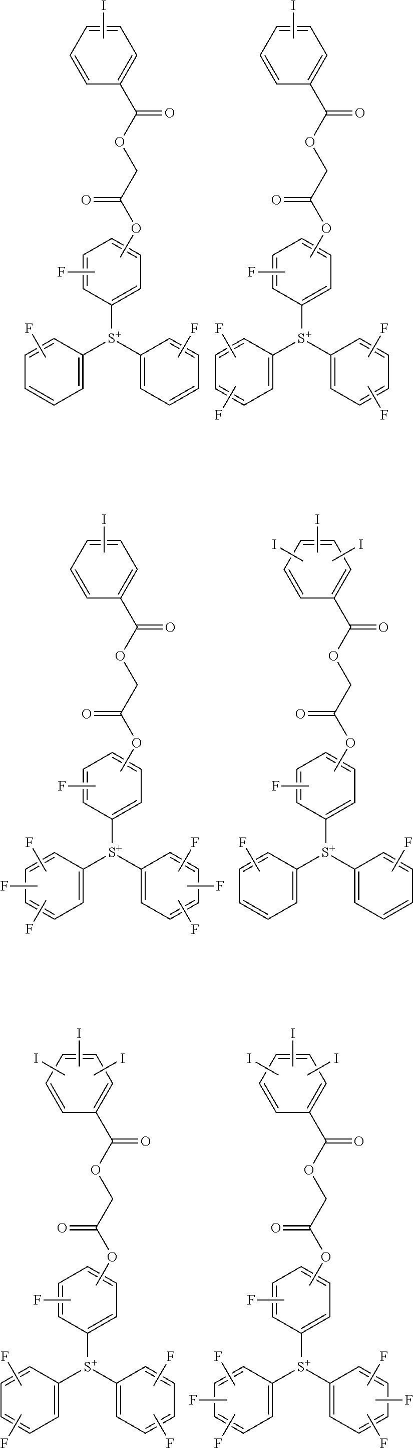

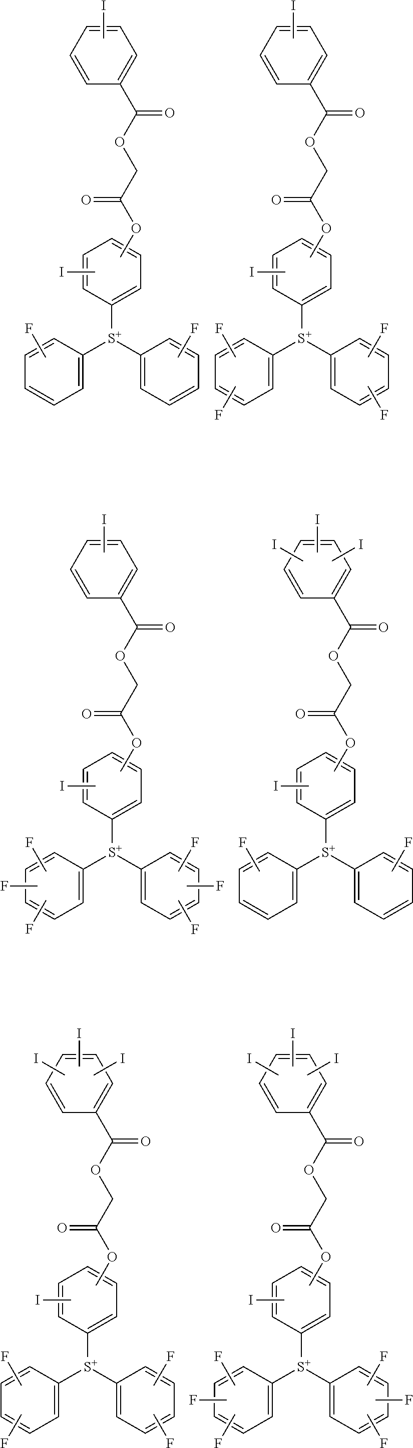

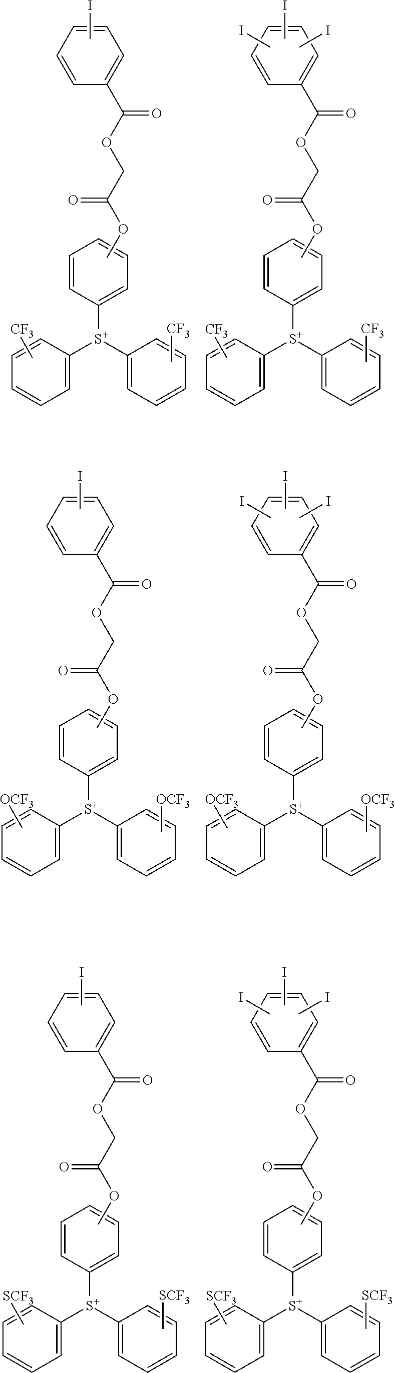

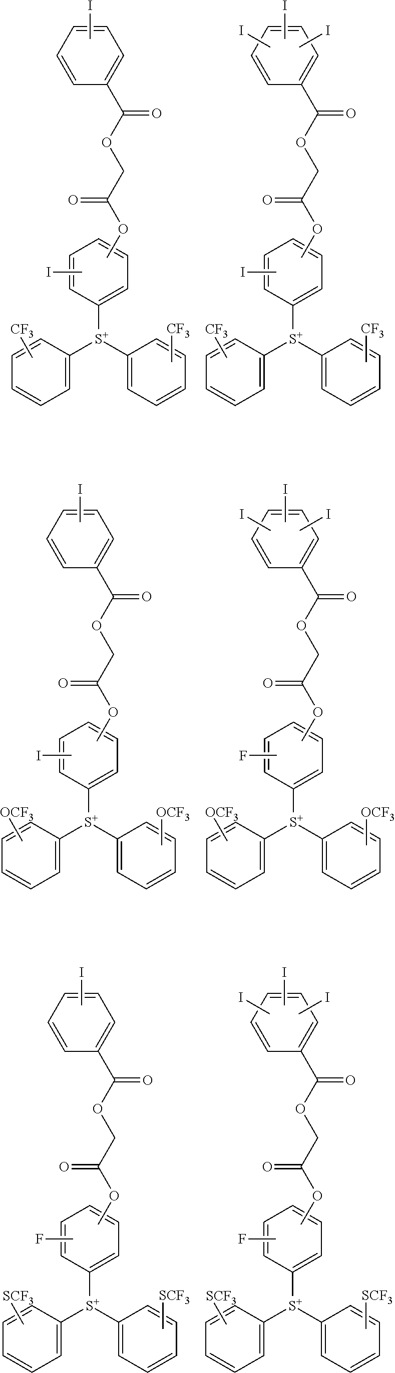

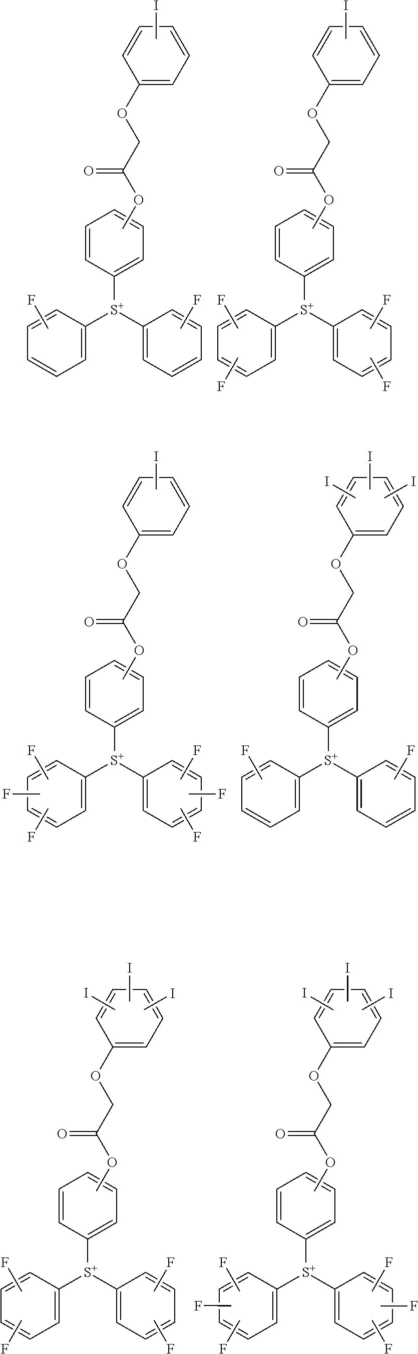

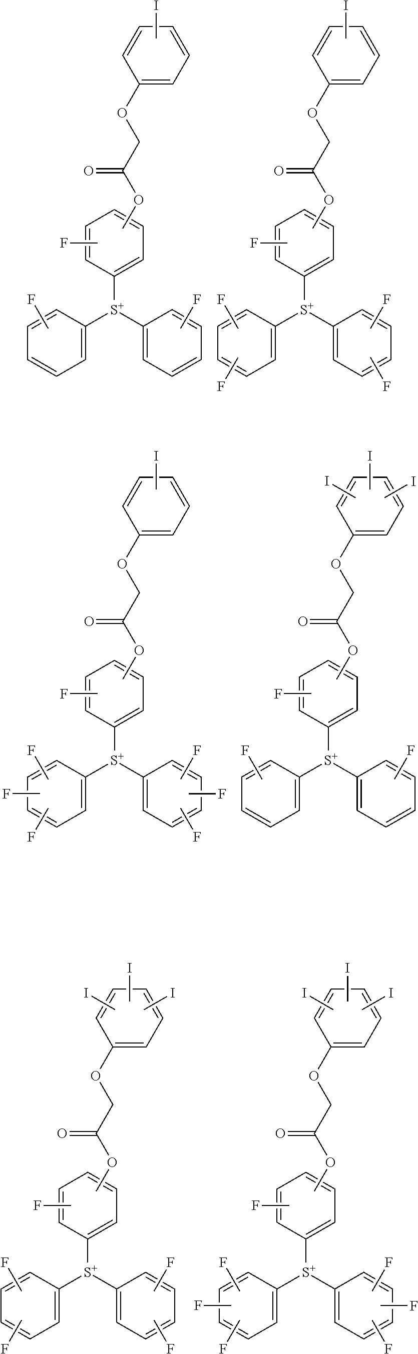

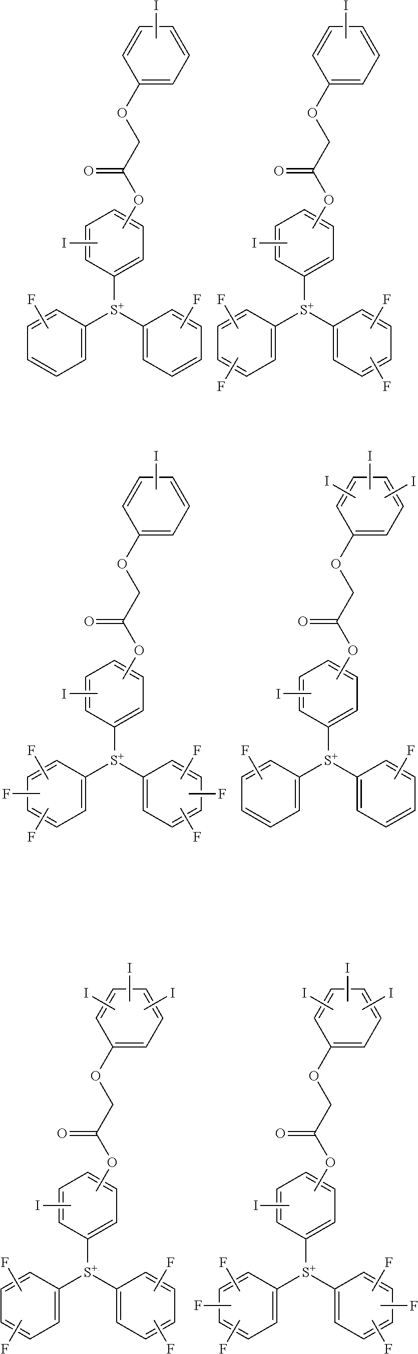

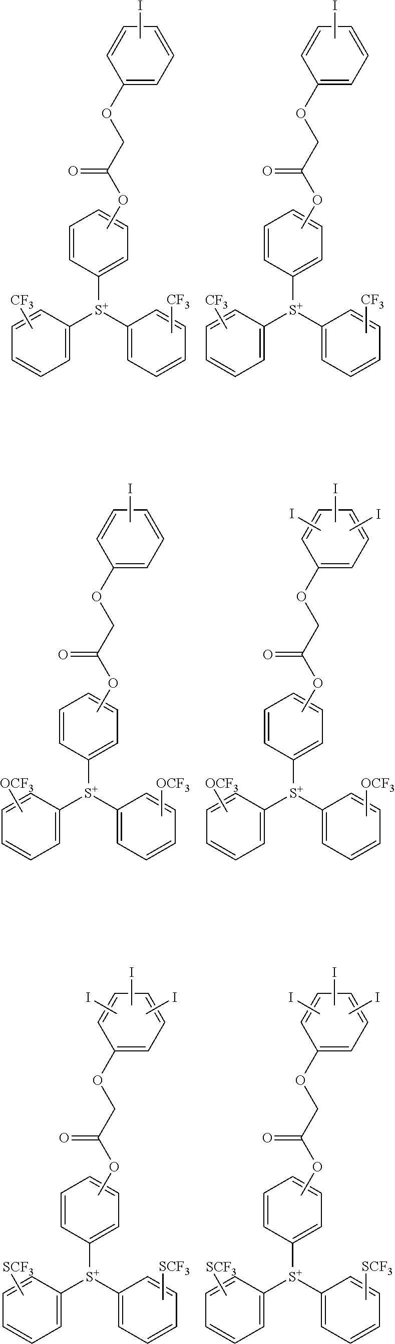

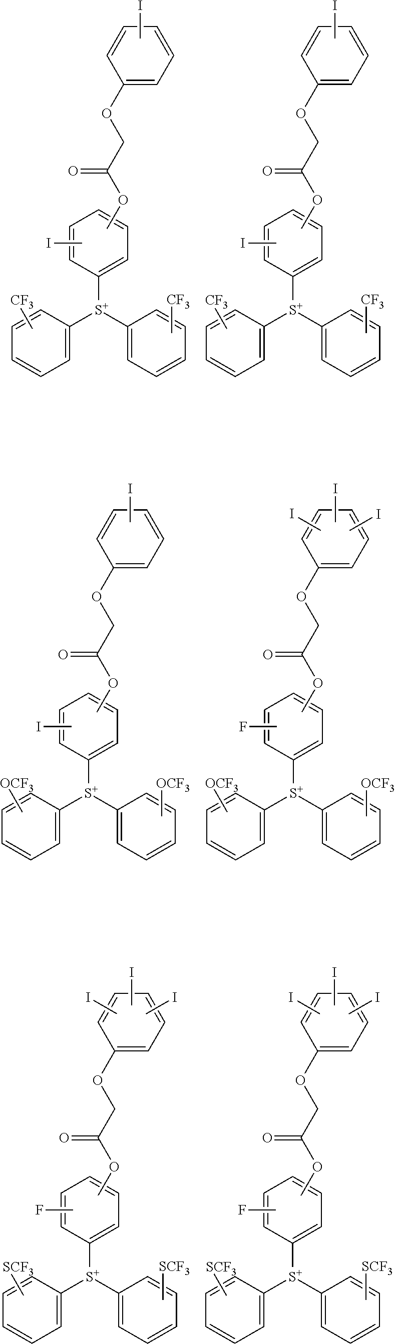

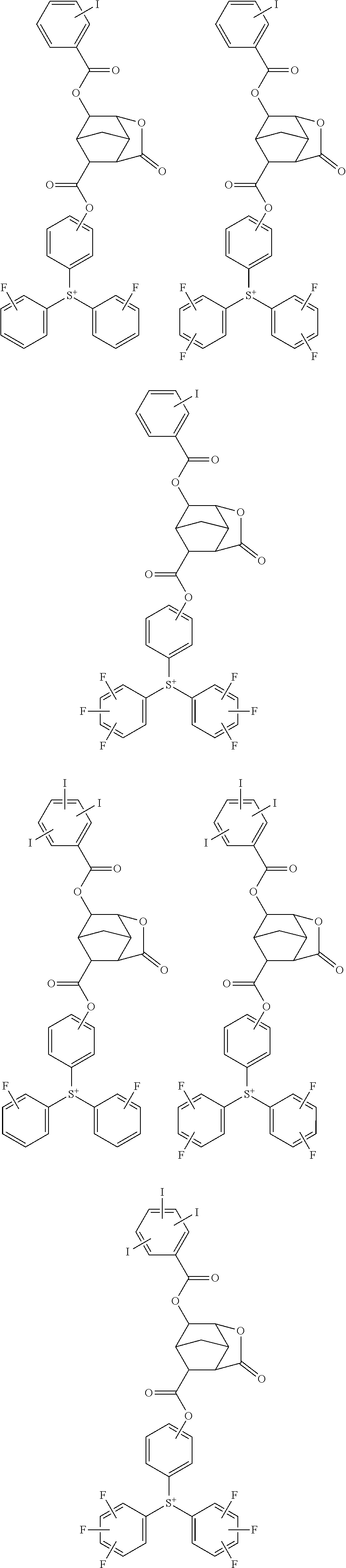

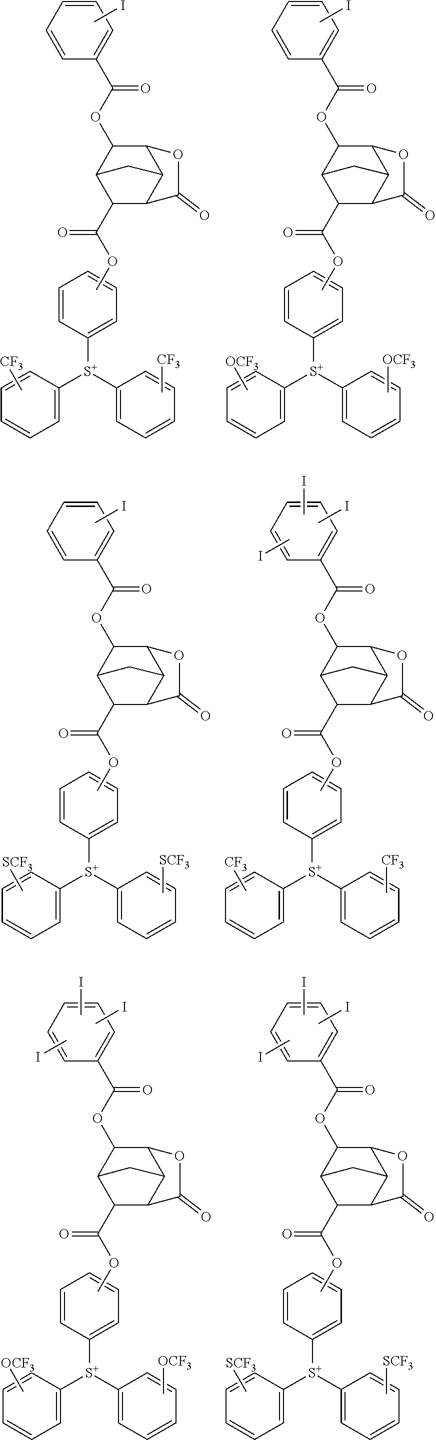

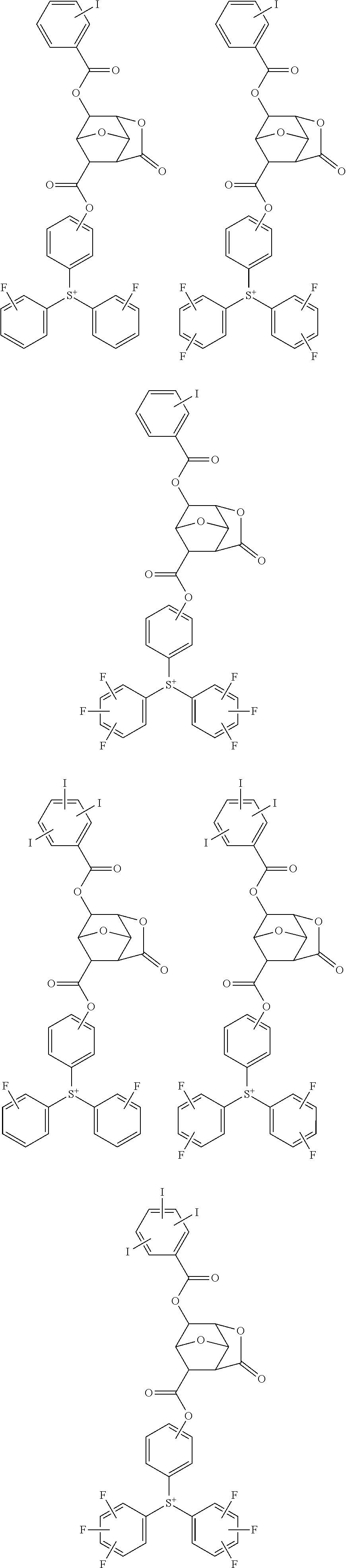

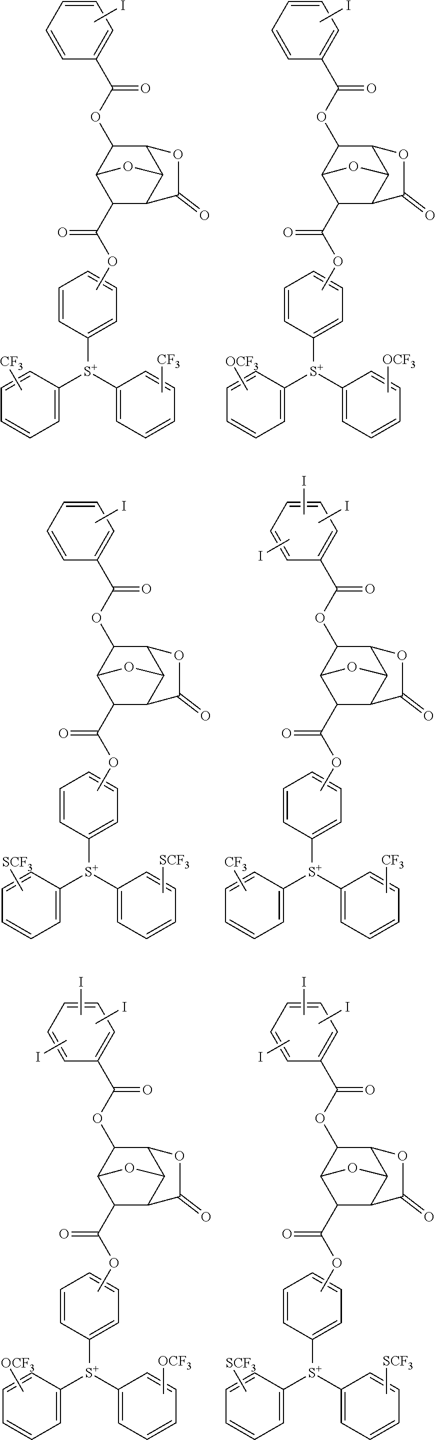

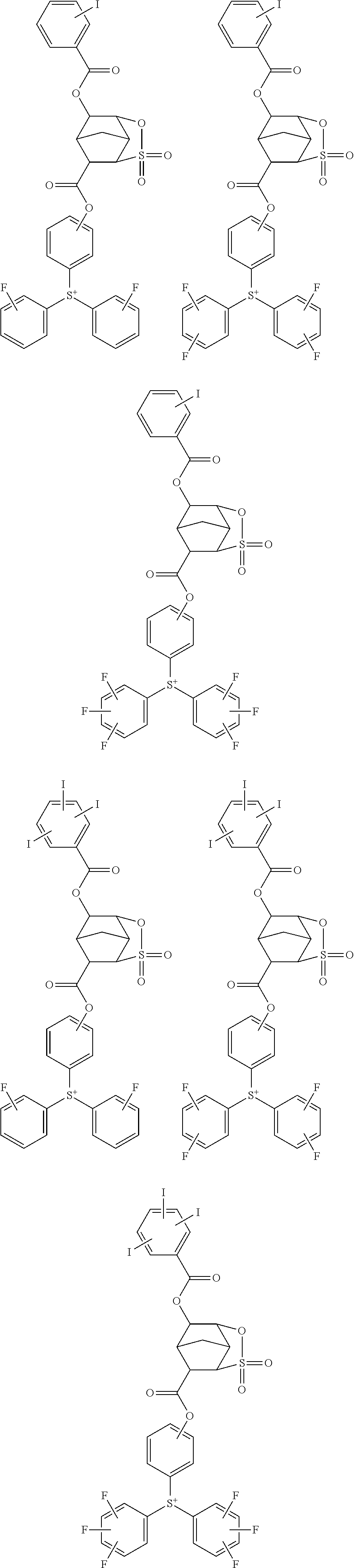

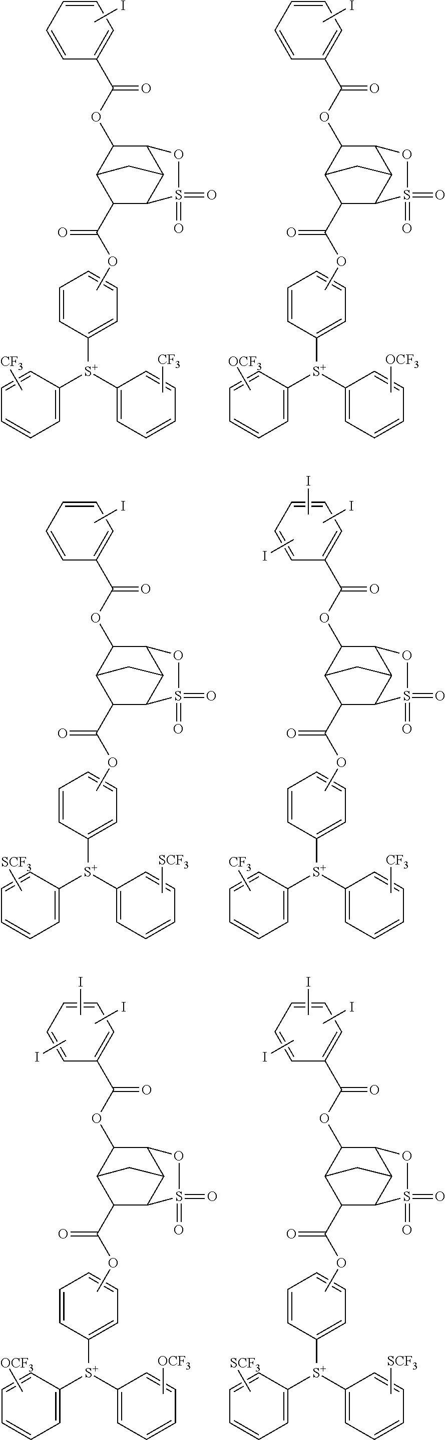

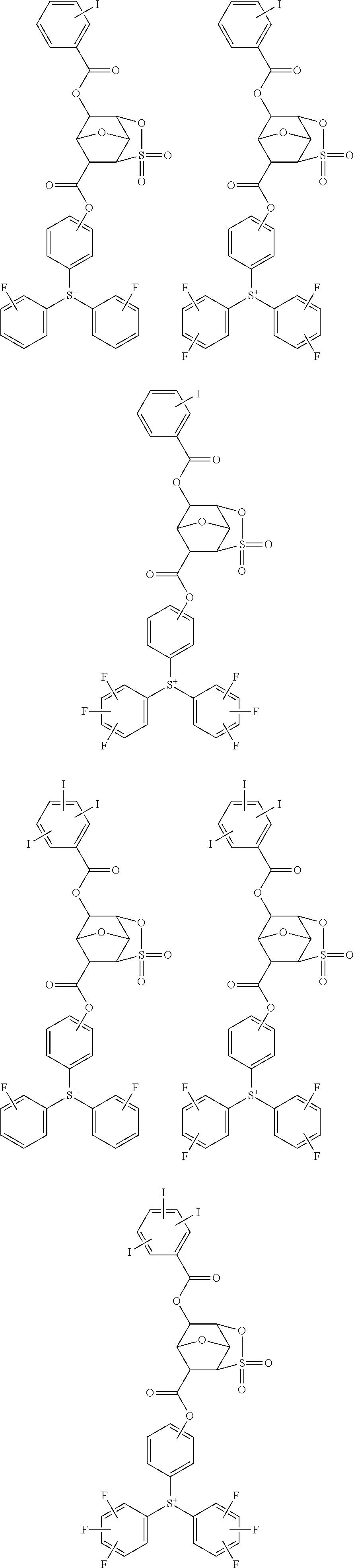

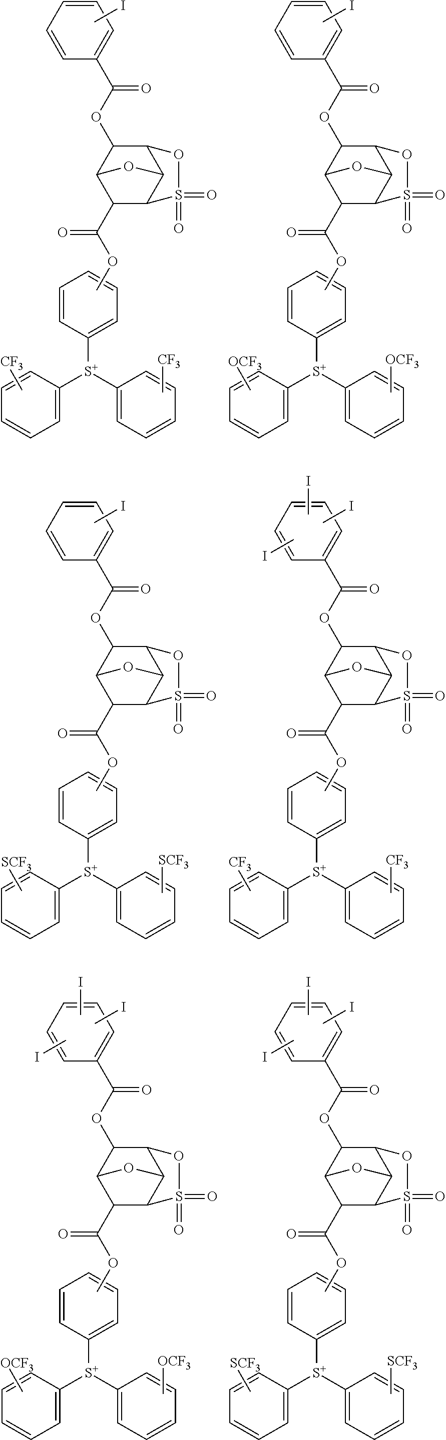

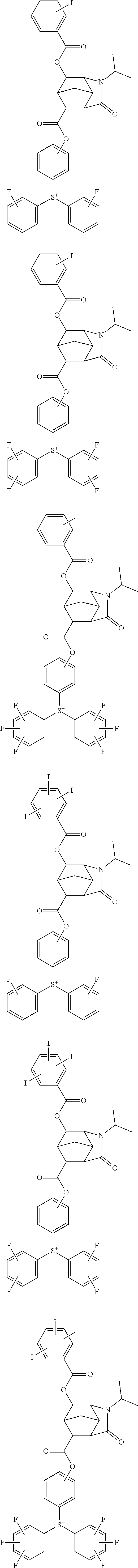

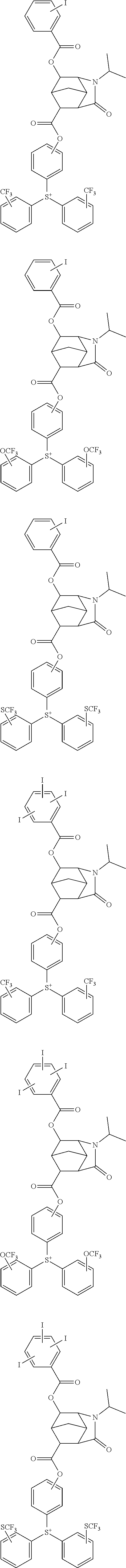

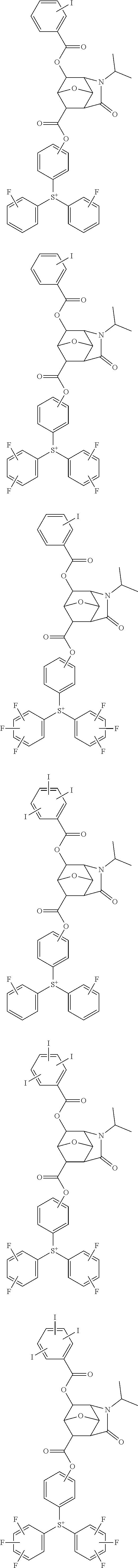

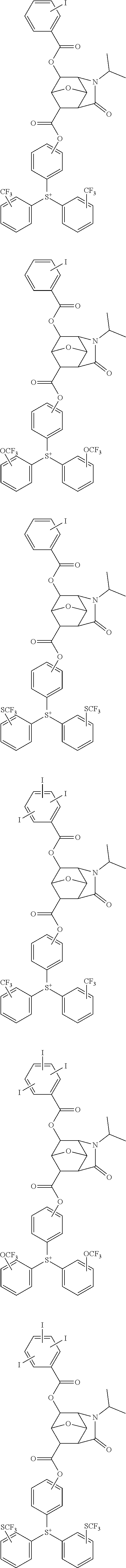

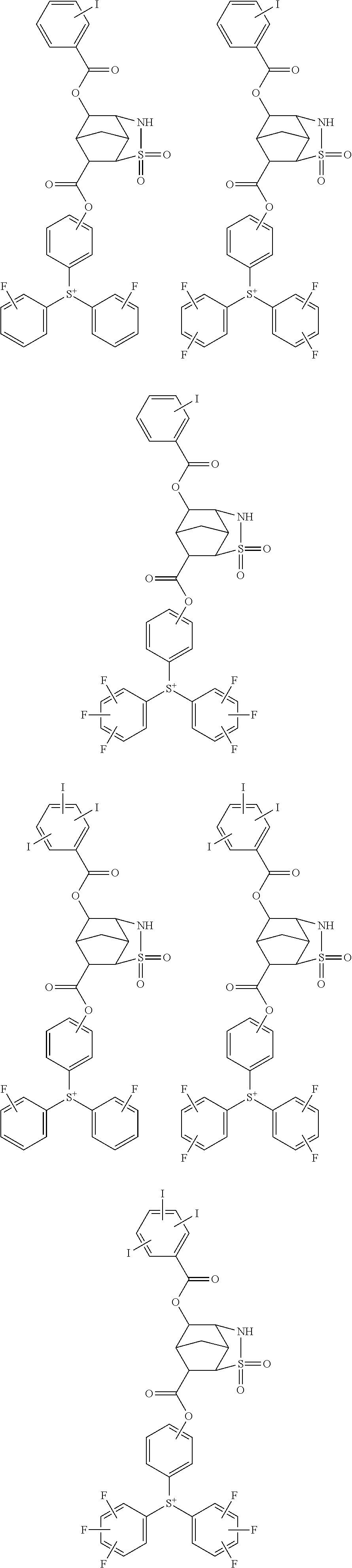

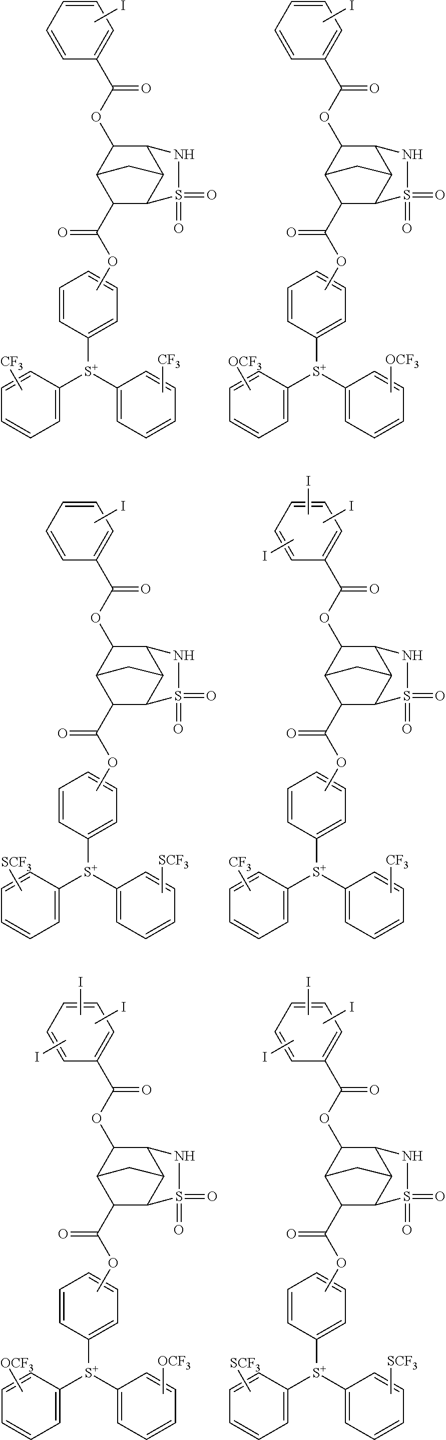

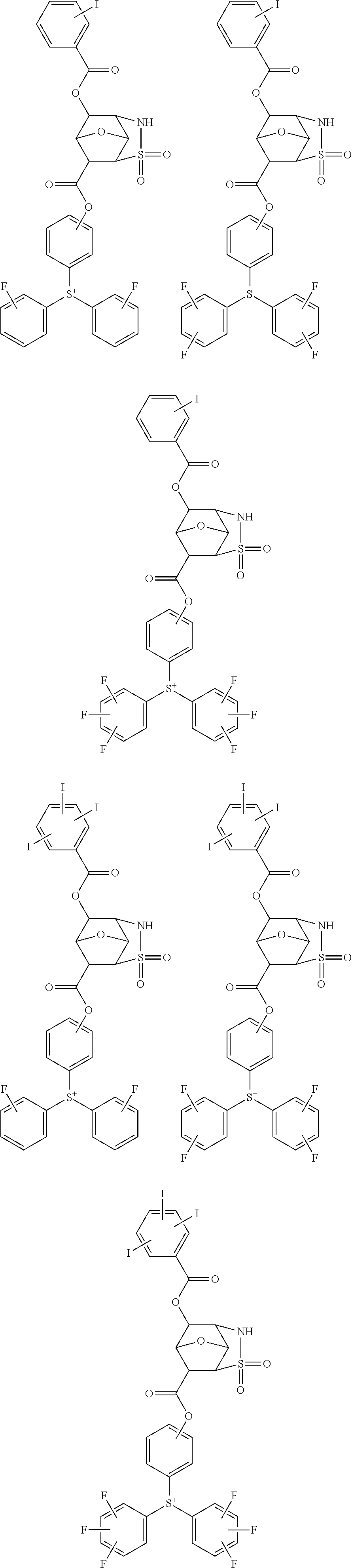

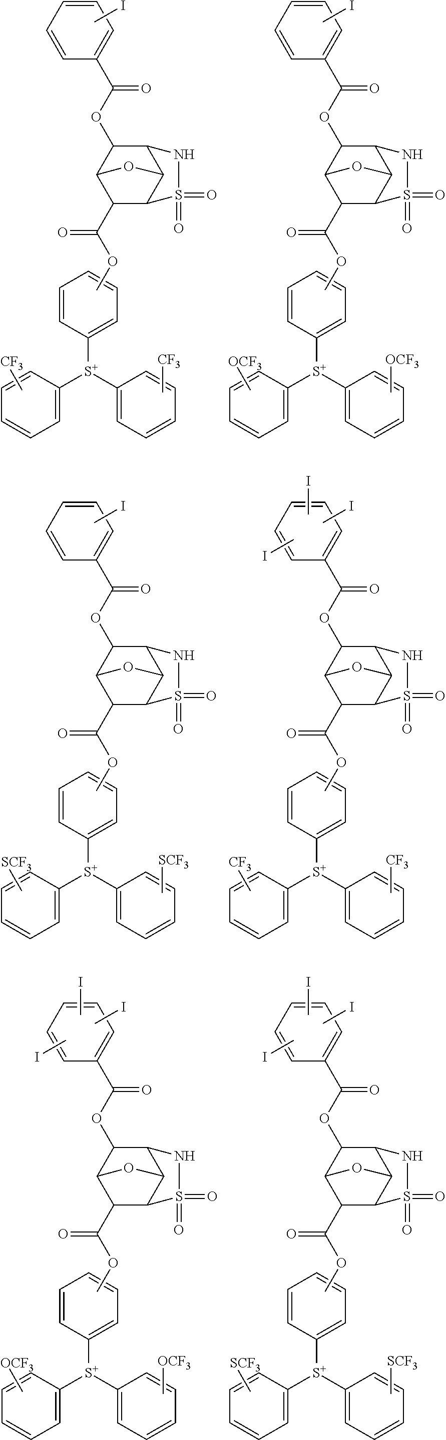

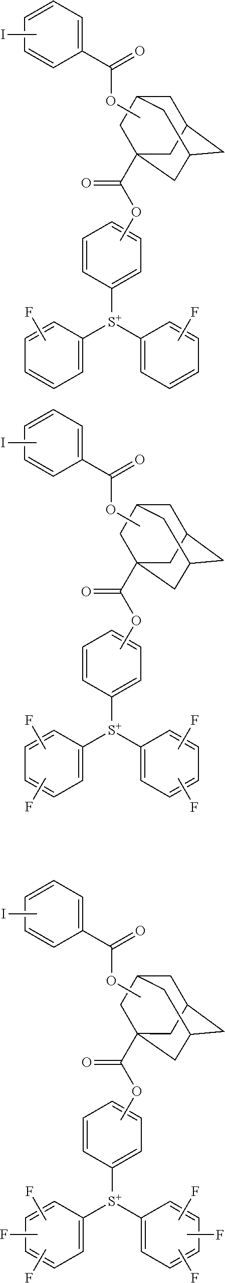

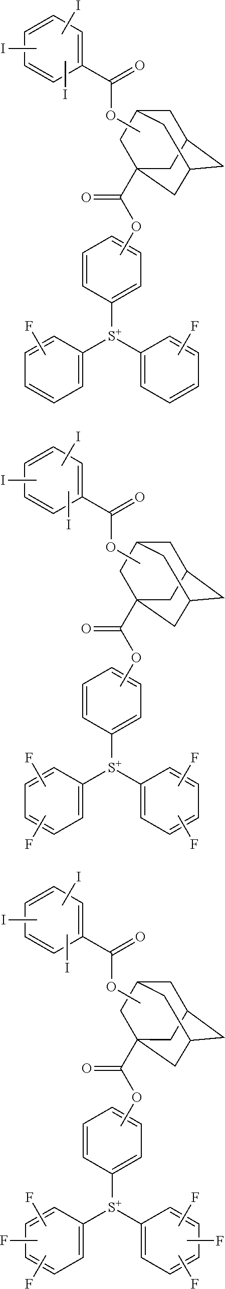

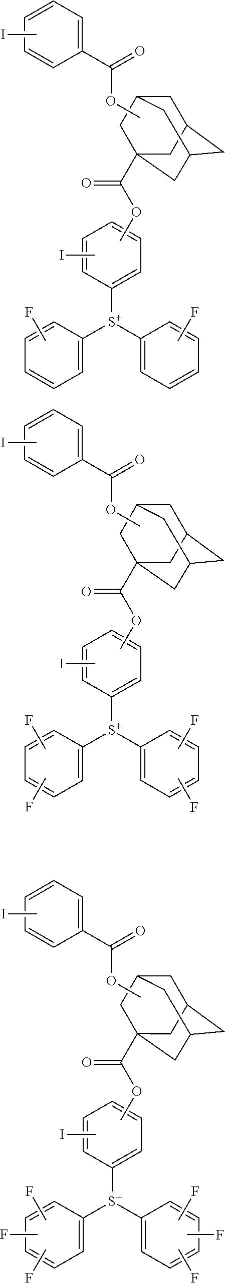

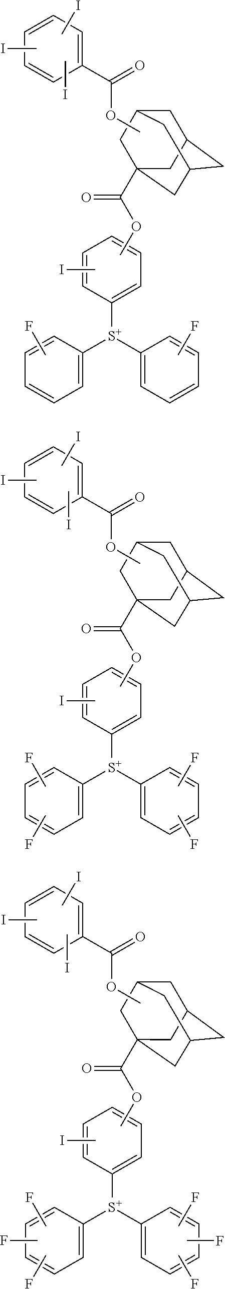

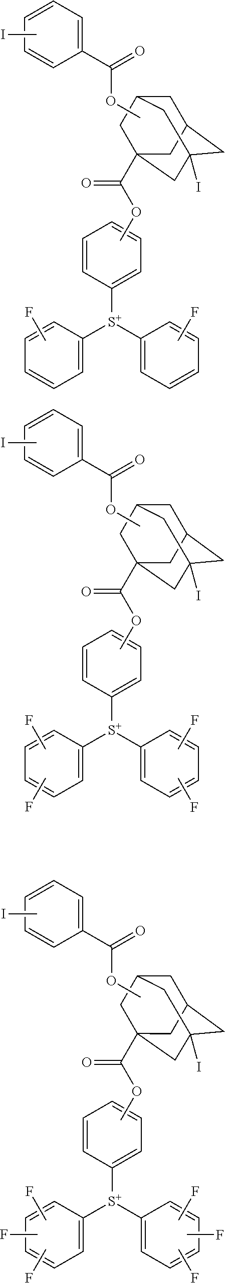

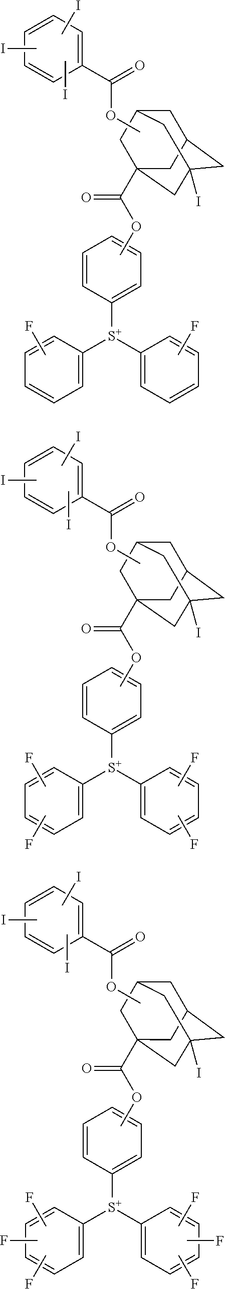

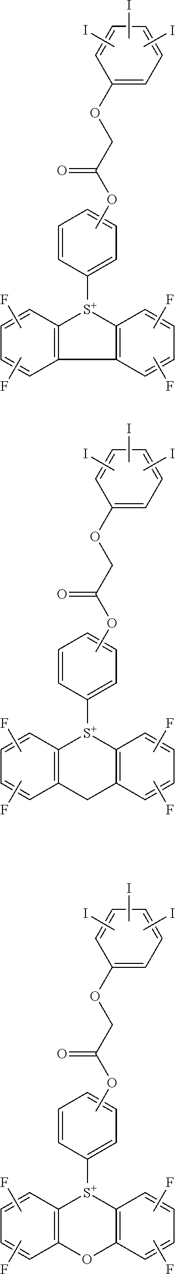

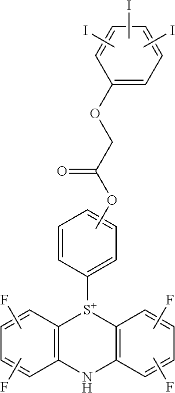

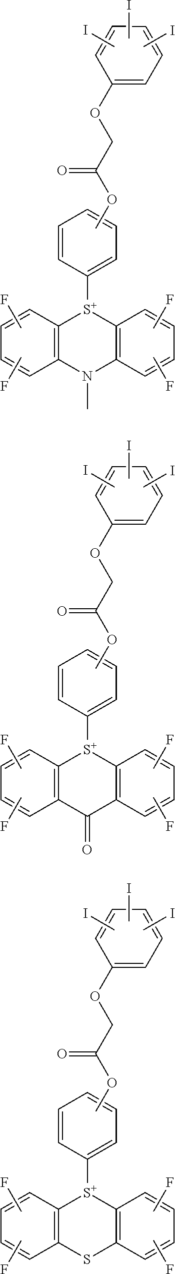

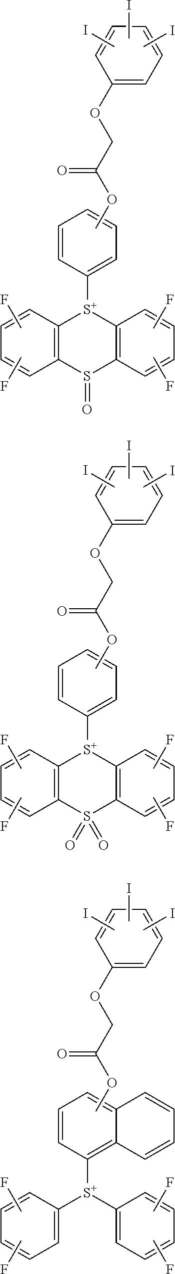

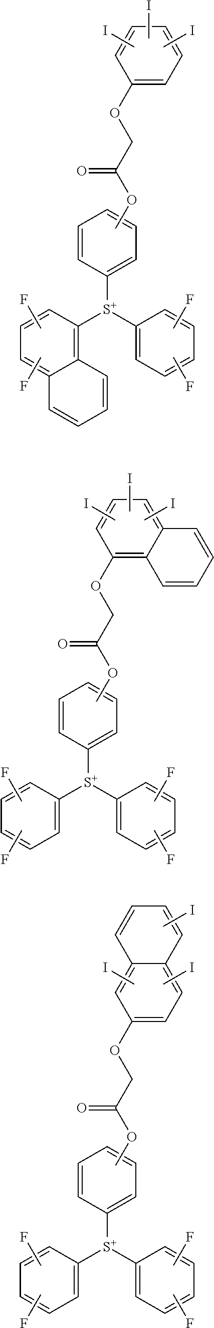

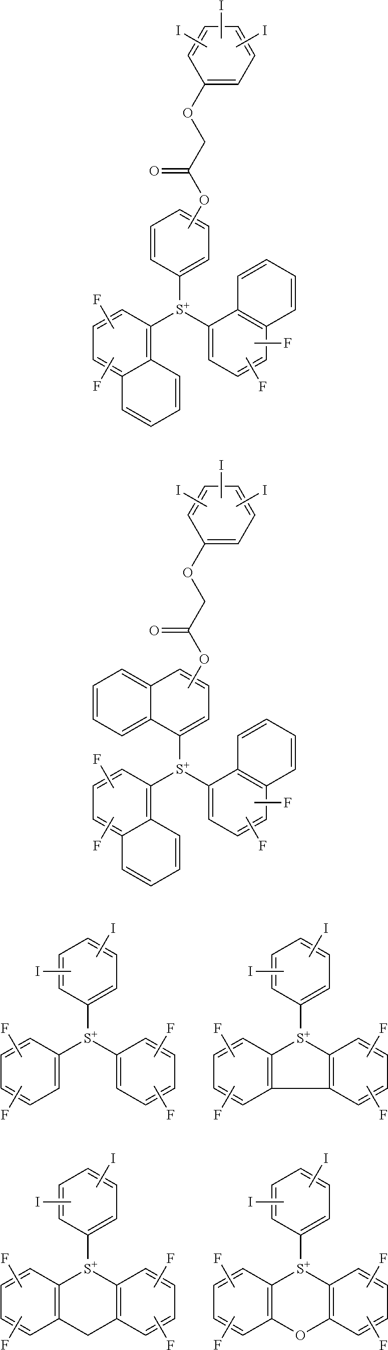

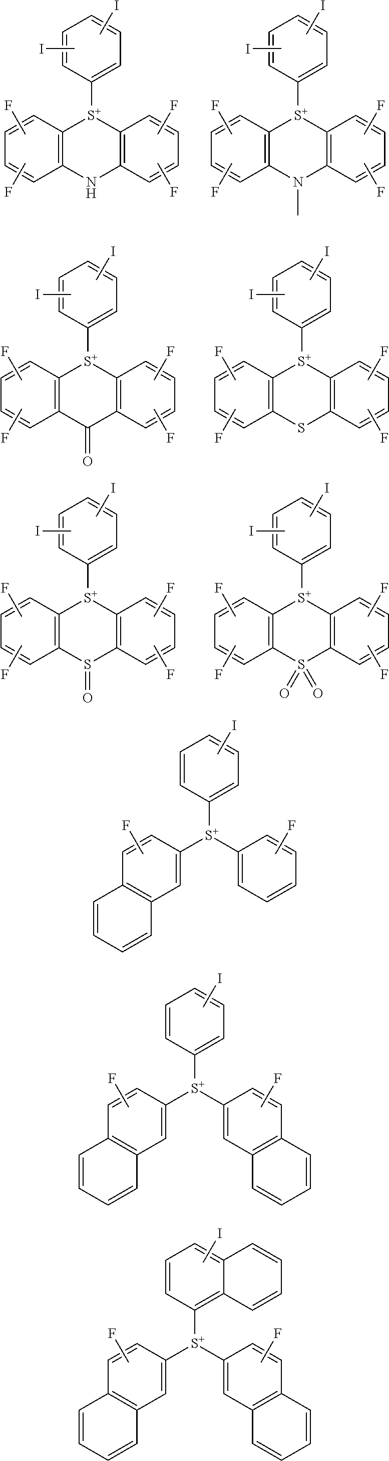

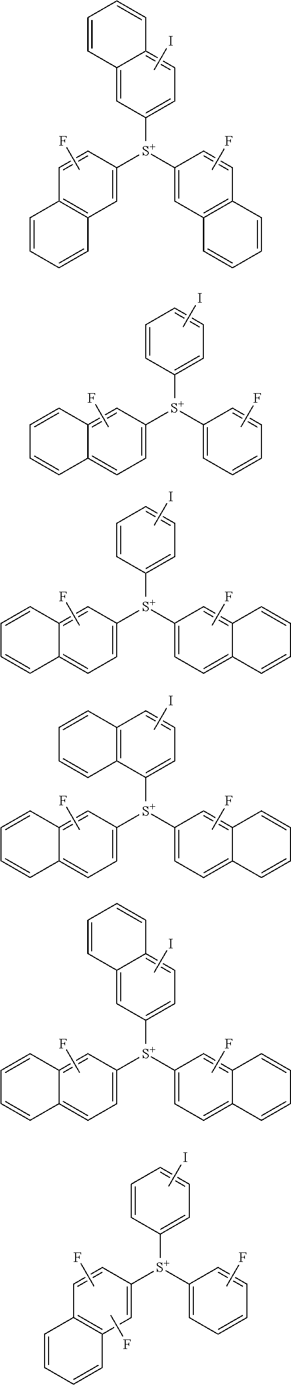

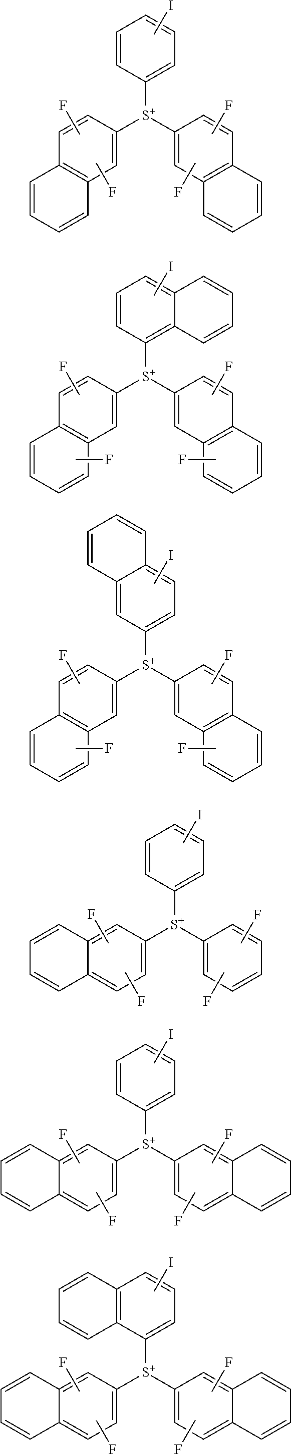

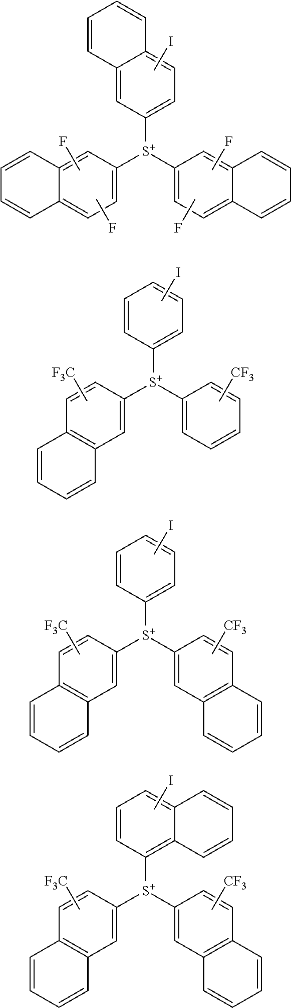

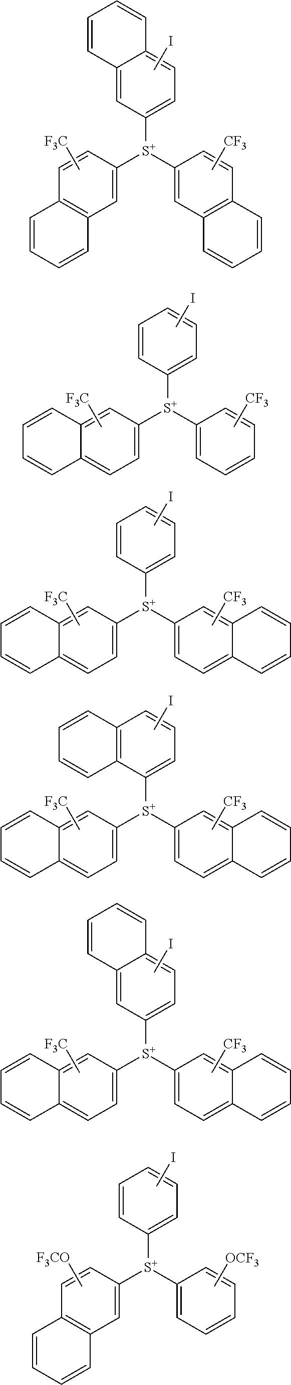

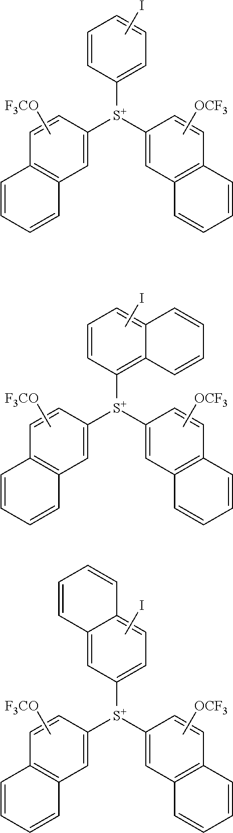

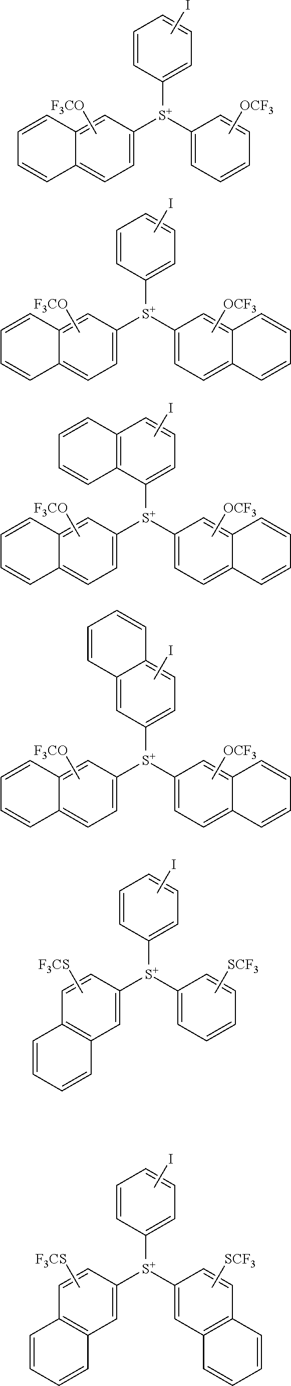

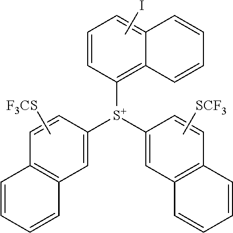

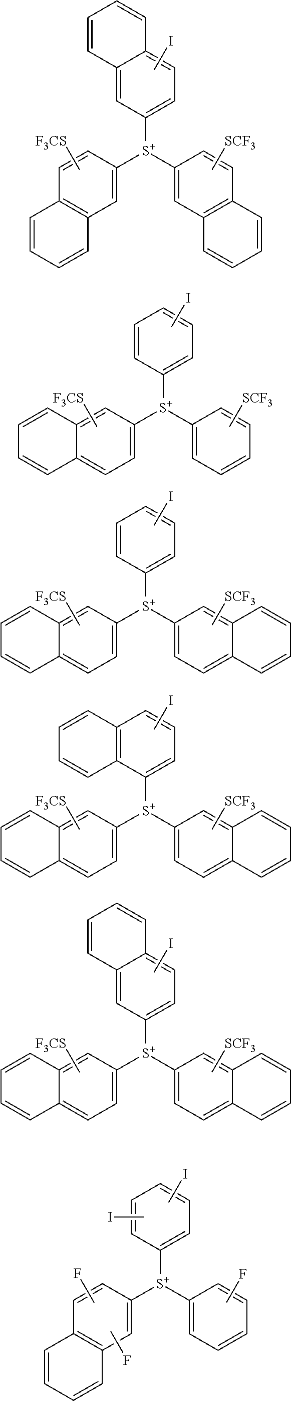

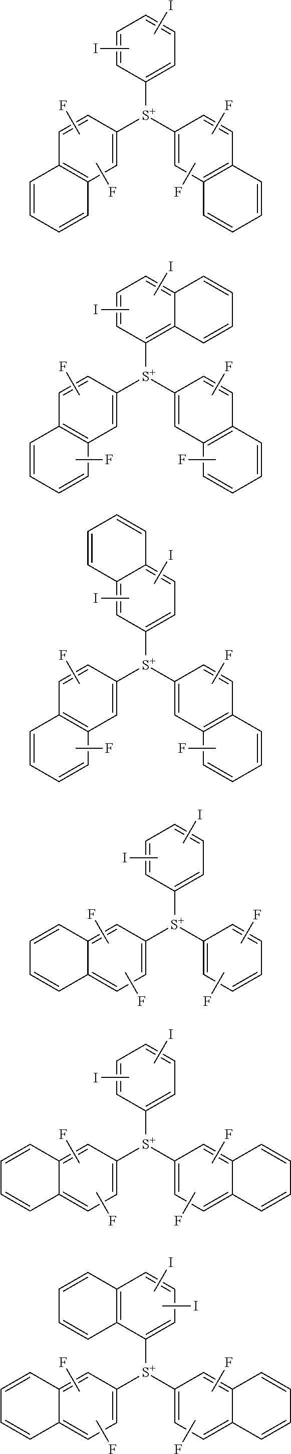

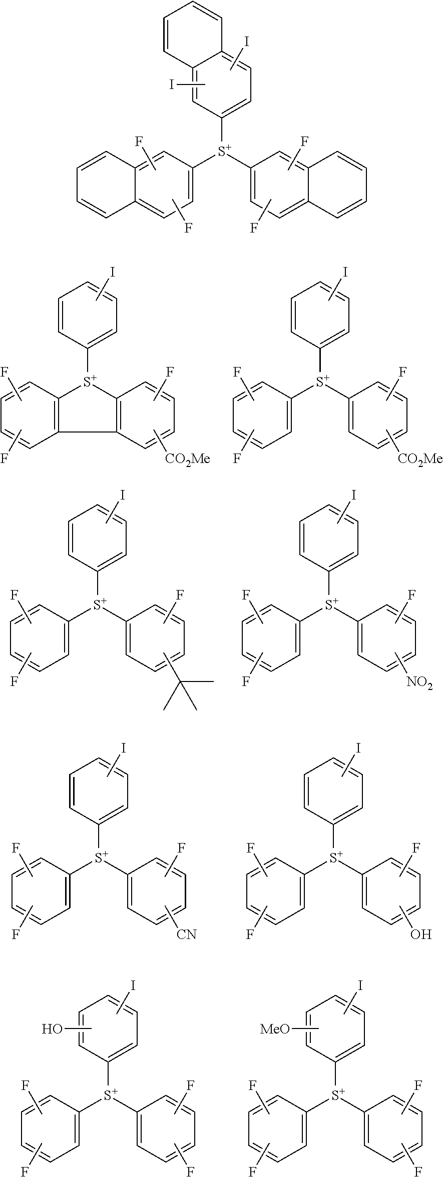

Specific examples of the onium salt of the present invention include any combination of the anion and the cation described above.

The onium salt monomer of the present invention can be synthesized by a known method. As an example, a method for manufacturing an onium salt monomer having the following formula (PAG-1-ex) is described, but a synthesis method is not limited thereto.

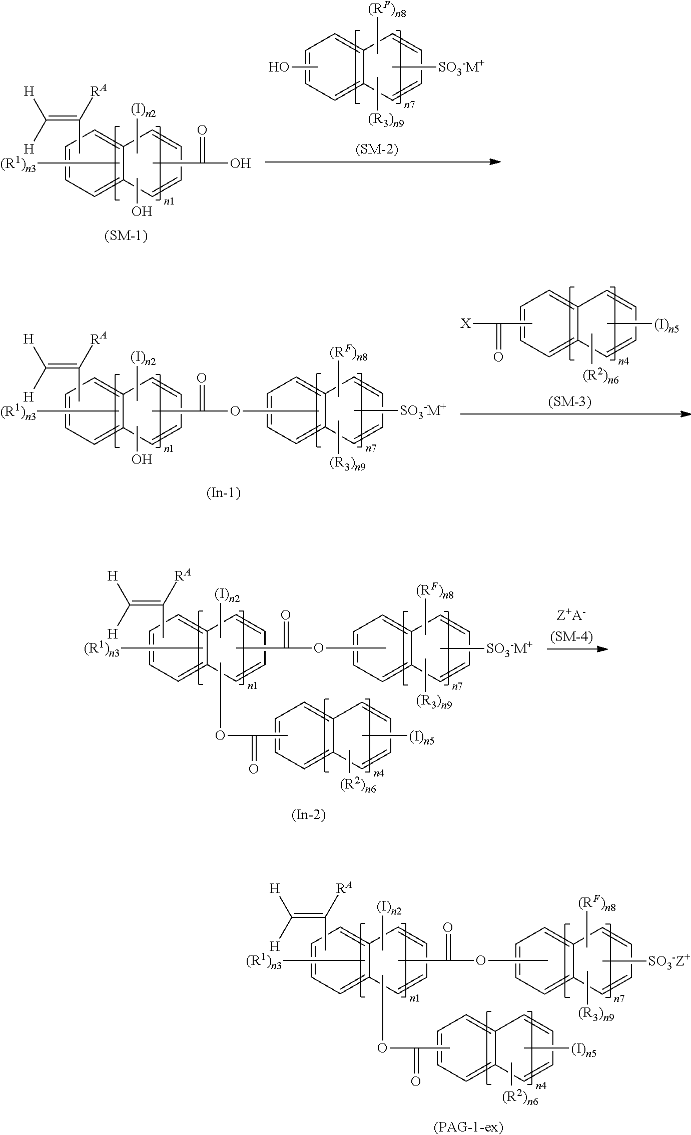

wherein n1 to n9, R^{A}, R¹ to R³, R^{F}, and Z⁺ are as defined above. M⁺ is a counter cation. A is a counter anion. X is a hydroxy group or a halogen atom selected from a chlorine atom, a bromine atom, and an iodine atom.

A first step is a step of performing a reaction between a raw material SM-1 and a raw material SM-2 which are commercially available products or obtained by known synthesis methods to obtain an intermediate In-1. When an ester bond is directly formed from a carboxy group of the raw material SM-1 and a hydroxy group of the raw material SM-2, various condensing agents can be used. Examples of the condensing agent to be used include N,N′-dicyclohexylcarbodiimide, N,N′-diisopropylcarbodiimide, 1-[3-(dimethylamino) propyl]-3-ethylcarbodiimide, and 1-ethyl-3-(3-dimethylaminopropyl) carbodiimide hydrochloride. It is preferable to use 1-ethyl-3-3-dimethylaminopropyl) carbodiimide hydrochloride from a viewpoint of ease of removing a urea compound generated as a by-product after the reaction. The reaction is performed by dissolving the raw material SM-1 and the raw material SM-2 in a halogen-based solvent such as methylene chloride and adding a condensing agent. When 4-dimethylaminopyridine (DMAP) is added as a catalyst, a reaction rate can be improved. It is desirable to complete the reaction by tracking the reaction by silica gel thin layer chromatography (TLC) from a viewpoint of yield, but a reaction time is usually about 12 to 24 hours. After the reaction is stopped, a urea compound as a by-product is removed by filtration or water washing as necessary, and then a reaction liquid is subjected to normal aqueous work-up, whereby the intermediate In-1 can be obtained. The obtained intermediate In-1 may be purified according to a conventional method such as chromatography or recrystallization as necessary.

A second step is a step of causing a raw material SM-3 to react with the obtained intermediate In-1 to obtain an intermediate In-2. When X is a hydroxy group, an ester bond is directly formed from a hydroxy group of the intermediate In-1 and a carboxy group of the raw material SM-3. At this time, various condensing agents can be used. Examples of the condensing agent to be used include those similar to those described in the first step. The reaction is performed by dissolving the intermediate In-1 and the raw material SM-3 in a halogen-based solvent such as methylene chloride and adding a condensing agent. When 4-dimethylaminopyridine (DMAP) is added as a catalyst, a reaction rate can be improved. When X is a halogen atom, an ester bond is formed from a hydroxy group of the intermediate In-1 and a carboxylic acid halide of the raw material SM-3. At this time, the reaction is preferably performed in the presence of an organic base such as triethylamine or pyridine, and 4-dimethylaminopyridine may be added for accelerating a reaction rate. The carboxylic acid halide is preferably a commercially available product or an acid chloride from a viewpoint of simplicity of preparation. A progress of the reaction is desirably confirmed by TLC from a viewpoint of yield, and a reaction time is usually about 12 to 24 hours. The intermediate In-2 can be obtained from the reaction mixture by normal aqueous work-up. If necessary, purification can be performed according to a conventional method such as chromatography or recrystallization.

A third step is a step of subjecting the obtained intermediate In-2 to salt exchange with an onium salt of Z⁺A⁻ (raw material SM-4) to obtain an onium salt (PAG-1-ex). Note that A is preferably a chloride ion, a bromide ion, an iodide ion, or a methyl sulfate anion because an exchange reaction easily proceeds quantitatively. It is desirable to complete the reaction by tracking the reaction by TLC from a viewpoint of yield, but a reaction time is usually about 4 to 12 hours. The onium salt (PAG-1-ex) can be obtained from the reaction mixture by normal aqueous work-up. If necessary, purification can be performed according to a conventional method such as chromatography or recrystallization.

In the scheme, the ion exchange in the third step can be easily performed by a known method, and for example, JP 2007-A-145797 can be referred to.

Note that the above manufacturing method is merely an example, and a method for manufacturing the onium salt of the present invention is not limited thereto.

### Polymer

A polymer of the present invention contains a repeat unit (hereinafter, also referred to as a repeat unit a) derived from the onium salt monomer having formula (a).

The polymer of the present invention is a polymer-bound photoacid generator that functions as a base polymer and as a photoacid generator in a chemically amplified resist composition. A structural feature of the onium salt monomer of the present invention is that an anion has a branched structure, which imparts good solvent solubility to the onium salt monomer. In addition, since the onium salt monomer has an aromatic ring substituted with a polymerizable group and an iodine atom, and has a structure in which a substituent containing an aromatic sulfonic acid anion structure and a substituent containing an aromatic ring substituted with an iodine atom are further bonded to the aromatic ring, a plurality of iodine atoms can be introduced thereinto. Since an iodine atom has very large absorption of EUV in EUV lithography of a wavelength of 13.5 nm, secondary electrons are generated from the iodine atom during exposure. Since the aromatic sulfonic acid anion extending from an aromatic vinyl structure has a short distance from a polymer main chain to an acid generation position, and is also at a position spatially close to the aromatic ring substituted with an iodine atom, bonded to the aromatic ring substituted with a polymerizable group and an iodine atom, secondary electrons generated from the iodine atom promote decomposition of a cation located in the vicinity of the anion, and an acid is efficiently generated, whereby sensitivity is increased. Furthermore, since an iodine atom has a large atomic weight and a generated acid is bonded to the polymer main chain, acid diffusion is small. The polymerizable group formed of styrene or a vinylnaphthalene structure is more rigid than a polymerizable group such as a methacrylate and improves a glass transition temperature (Tg) of the polymer. It is considered that aromatic rings in the base polymer or between the base polymers interact with each other (π-π stacking effect), whereby the base polymers are regularly arranged, and resistance to pattern collapse is exhibited against a developer even during fine pattern formation. Also in an etching step after fine pattern formation, excellent etching resistance is exhibited by an aromatic ring directly connected to the main chain. When a triarylsulfonium cation containing an iodine atom and a fluorine atom is used as the cation, secondary electrons are similarly generated from the iodine atom. In addition, an energy level of LUMO in a frontier orbital theory is lowered by an electron attraction effect by a fluorine atom, and generated secondary electrons are easily received. Therefore, decomposition of the cation is promoted, and an acid is effectively generated. These synergistic effects increase sensitivity. This makes it possible to prevent a decrease in resolution due to blurring of acid diffusion and to improve LWR and CDU. Thus, the polymer of the present invention is suitable particularly as a material for a chemically amplified positive resist composition.

The polymer may further contain a repeat unit having the following formula (b1) (hereinafter, also referred to as a repeat unit b1) or a repeat unit having the following formula (b2) (hereinafter, also referred to as a repeat unit b2).

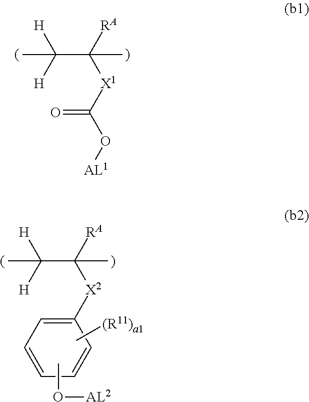

In formulas (b1) and (b2), RAs are each independently a hydrogen atom, a fluorine atom, a methyl group, or a trifluoromethyl group.

In formula (b1), X¹ is a single bond, a phenylene group, a naphthylene group, *—C(═O)—O—X¹¹—, or *—C(═O)—NH—X¹¹—, and the phenylene group or the naphthylene group may be substituted with a hydroxy group, a nitro group, a cyano group, a C₁-C₁₀ saturated hydrocarbyl group which may contain a fluorine atom, a C₁-C₁₀ saturated hydrocarbyloxy group which may contain a fluorine atom, or a halogen atom. X¹¹ is a C₁-C₁₀ saturated hydrocarbylene group, a phenylene group, or a naphthylene group, and the saturated hydrocarbylene group may contain a hydroxy group, an ether bond, an ester bond, or a lactone ring. * designates a point of attachment to the carbon atom of the main chain.

In formula (b2), X² is a single bond, *—C(═O)—O—, or —C(═O)—NH—. * designates a point of attachment to the carbon atom of the main chain. R¹¹ is a halogen atom, a cyano group, a hydroxy group, a nitro group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom. a1 is 0, 1, 2, 3, or 4, and preferably 0 or 1.

In formulas (b1) and (b2), AL¹ and AL² are each independently an acid-unstable group. Specific examples of the acid-unstable group include, but are not limited to, those described in JP 2013-A-80033 and JP 2013-A-83821.

Typically, specific examples of the acid-unstable group include those having the following formulas (AL-1) to (AL-3):

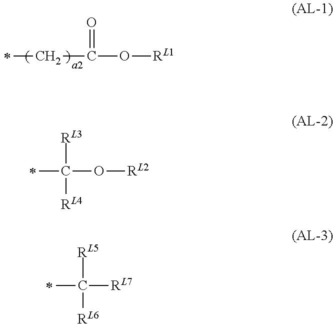

wherein * designates a point of attachment.

In formulas (AL-1) and (AL-2), R^{L1} and R¹² are each independently a C₁-C₄₀ hydrocarbyl group, and may contain a heteroatom such as an oxygen atom, a sulfur atom, a nitrogen atom, a fluorine atom, or an iodine atom. The hydrocarbyl group may be saturated or unsaturated, and may be linear, branched, or cyclic. The hydrocarbyl group preferably has 1 to 20 carbon atoms.

In formula (AL-1), a2 is an integer of 0 to 10, and preferably 1, 2, 3, 4 or 5.

In formula (AL-2), R^{L3} and R^{L4} are each independently a hydrogen atom or a C₁-C₂₀ hydrocarbyl group, and may contain a heteroatom such as an oxygen atom, a sulfur atom, a nitrogen atom, a fluorine atom, or an iodine atom. The hydrocarbyl group may be saturated or unsaturated, and may be linear, branched, or cyclic. Any two of R^{L2}, R^{L3}, and R^{L4} may be bonded to each other to form a C₃-C₂₀ ring together with a carbon atom or a carbon atom and an oxygen atom to which they are bonded. The ring is preferably a C₄-C₁₆ ring, and particularly preferably an alicyclic ring.

In formula (AL-3), R^{L5}, R^{L6}, and R^{L7} are each independently a C₁-C₂₀ hydrocarbyl group, and may contain a heteroatom such as an oxygen atom, a sulfur atom, a nitrogen atom, a fluorine atom, or an iodine atom. The hydrocarbyl group may be saturated or unsaturated, and may be linear, branched, or cyclic. Any two of R^{L5}, R^{L6}, and R^{L7} may be bonded to each other to form a C₃-C₂₀ ring together with a carbon atom to which they are bonded. The ring is preferably a C₄-C₁₆ ring, and particularly preferably an alicyclic ring.

Specific examples of the repeat unit b1 include, but are not limited to, those listed below. Note that, in the following formulas, R^{A} and AL¹ are as defined above.

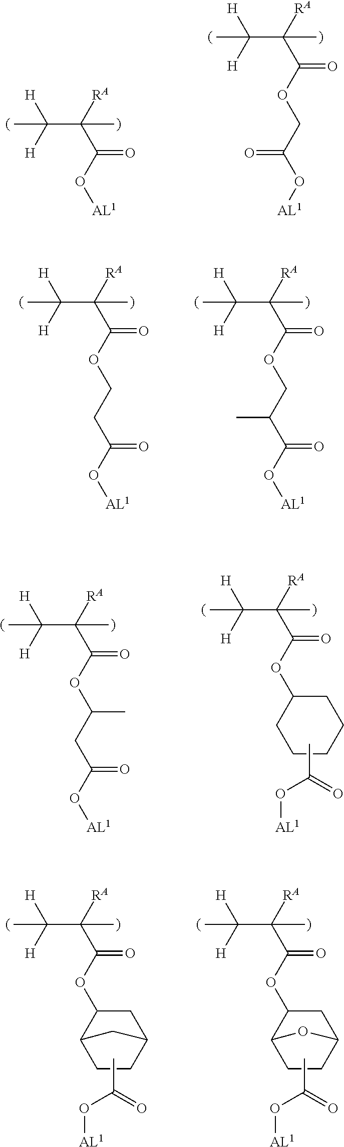

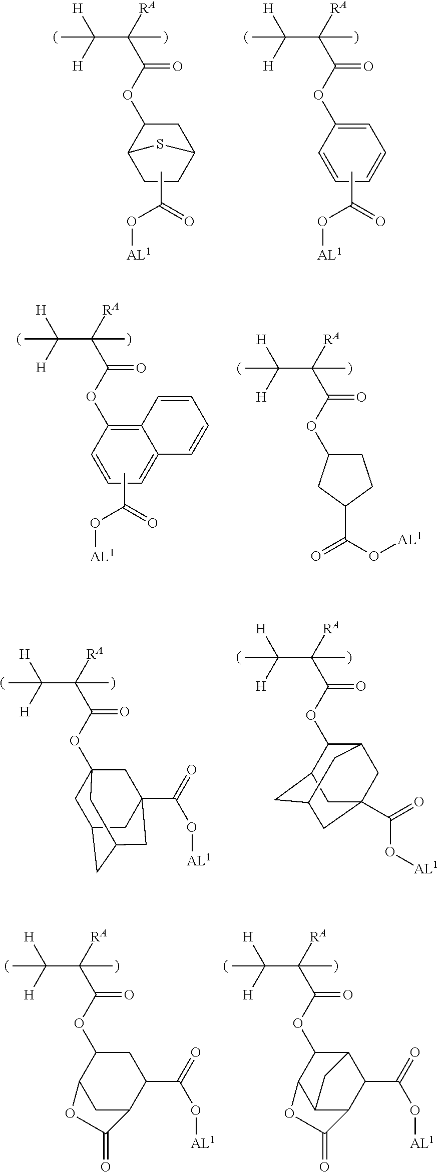

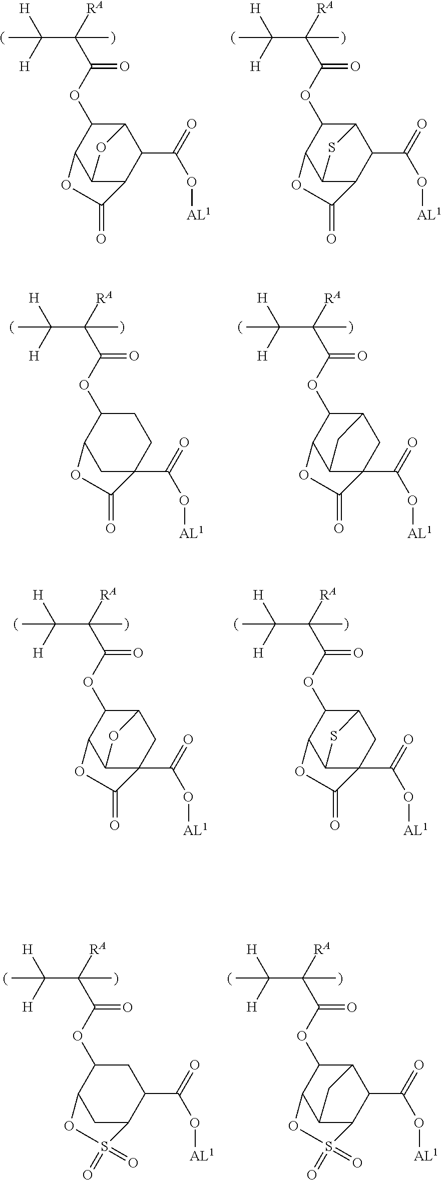

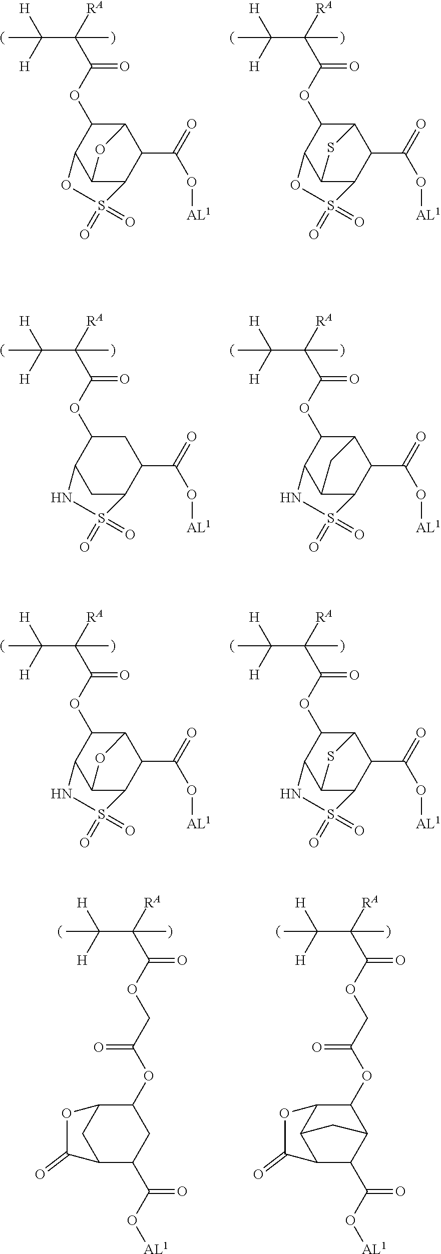

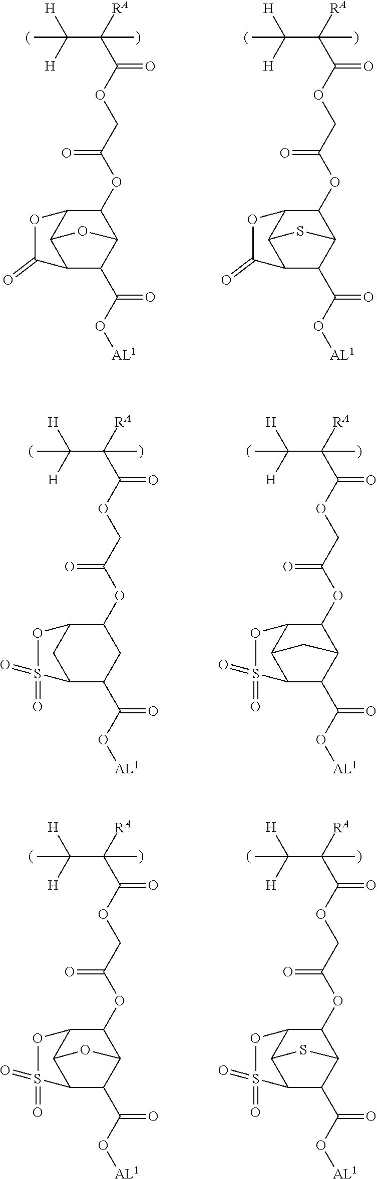

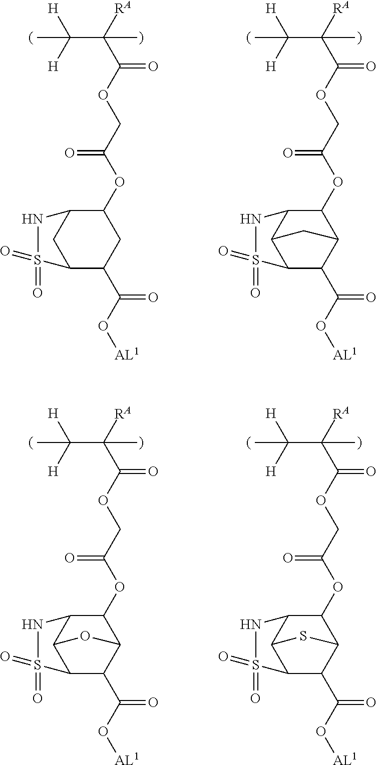

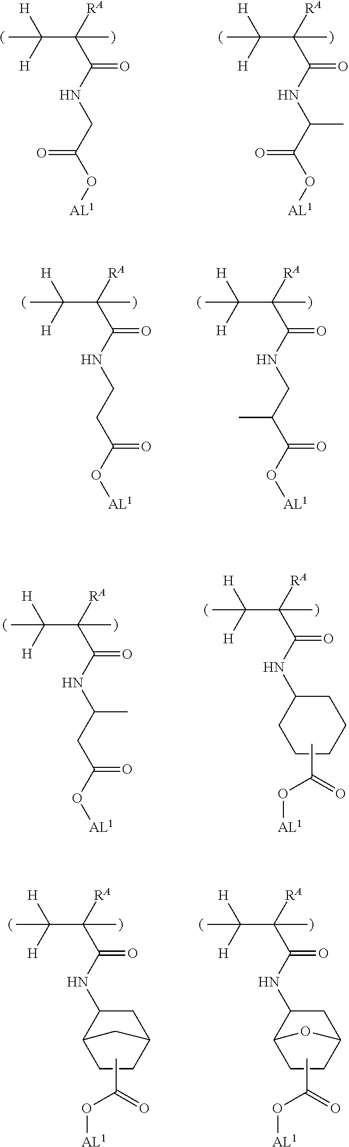

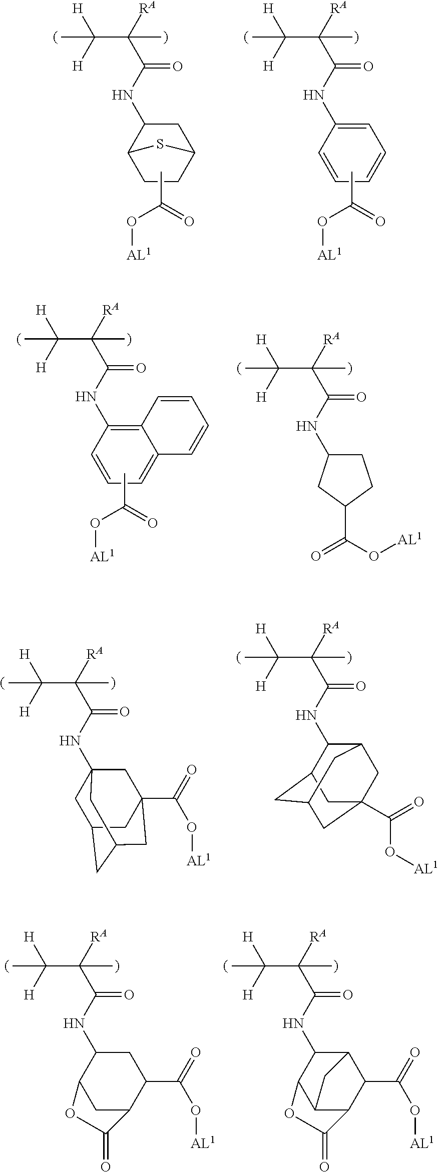

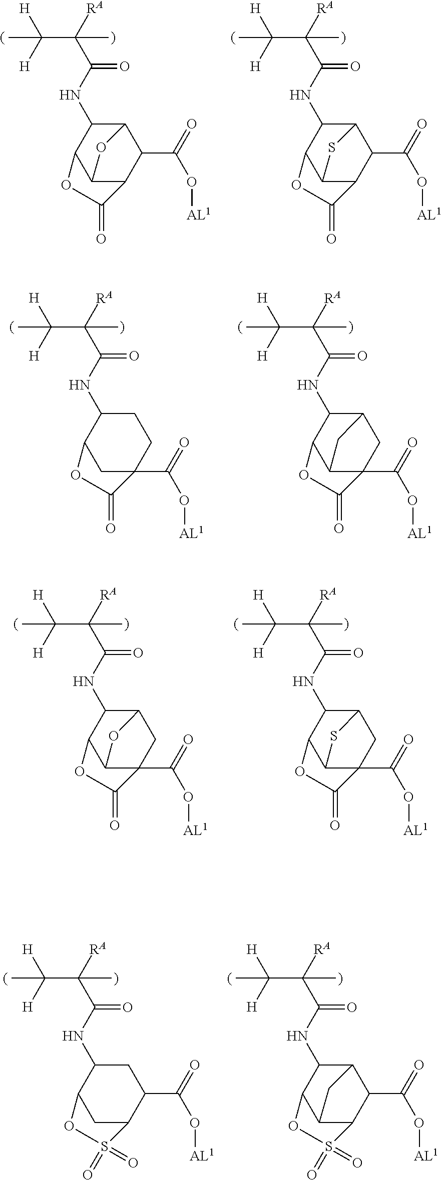

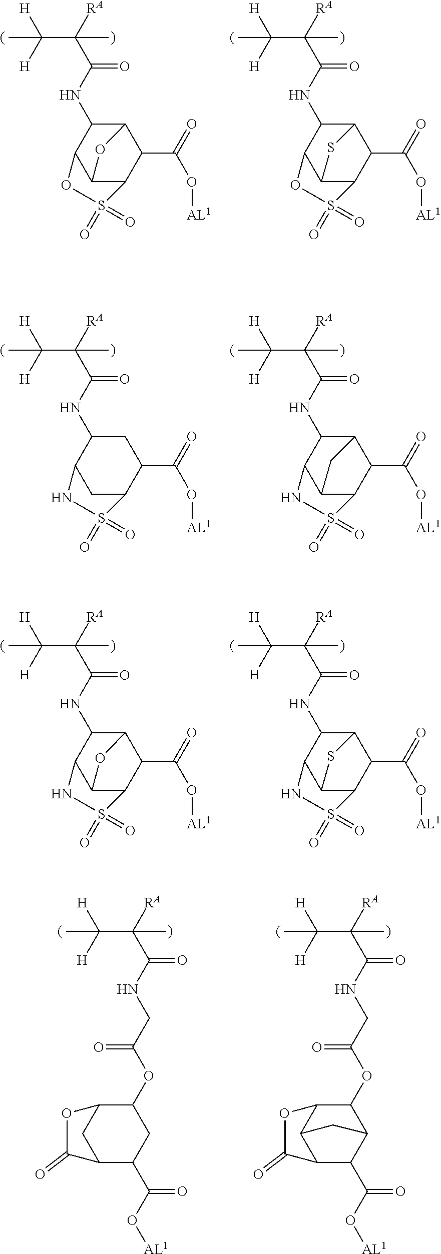

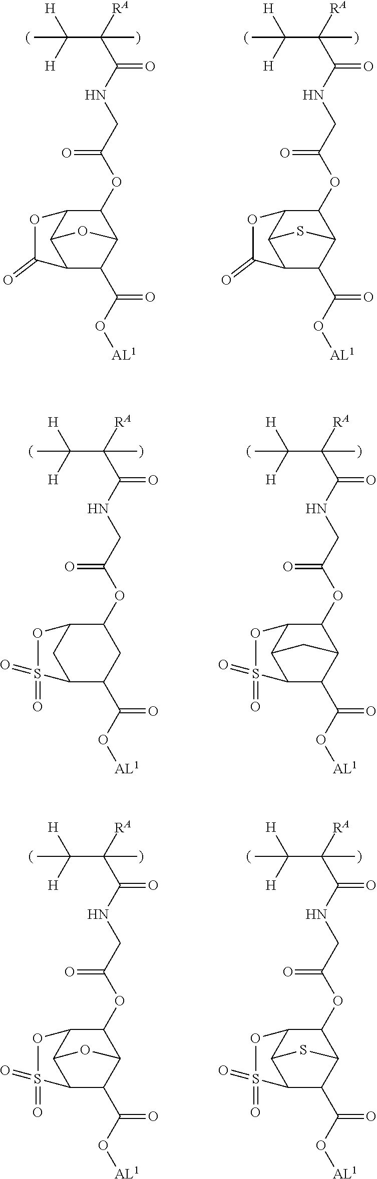

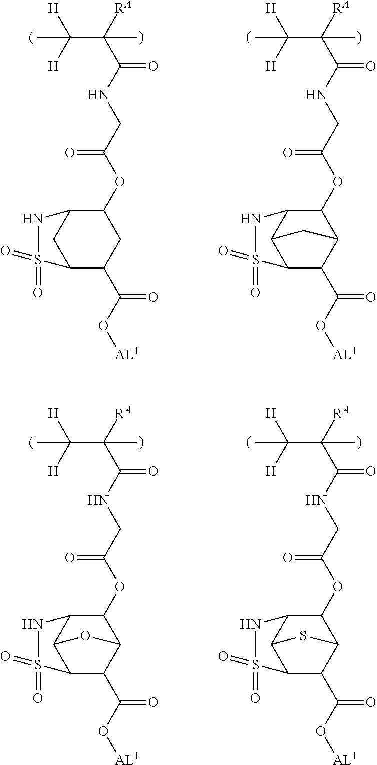

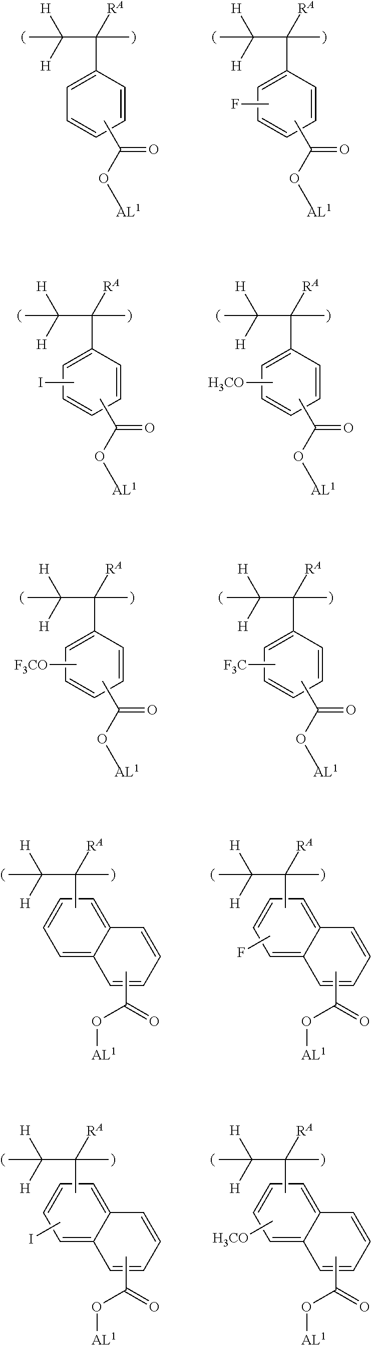

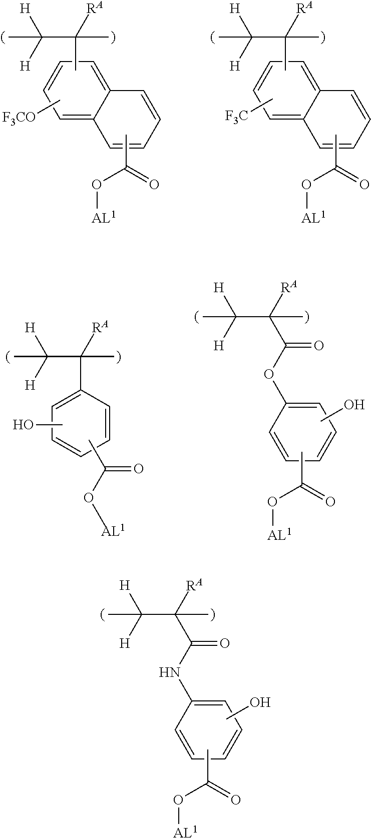

Specific examples of the repeat unit b2 include, but are not limited to, those listed below. Note that, in the following formulas, R^{A} and AL² are as defined above.

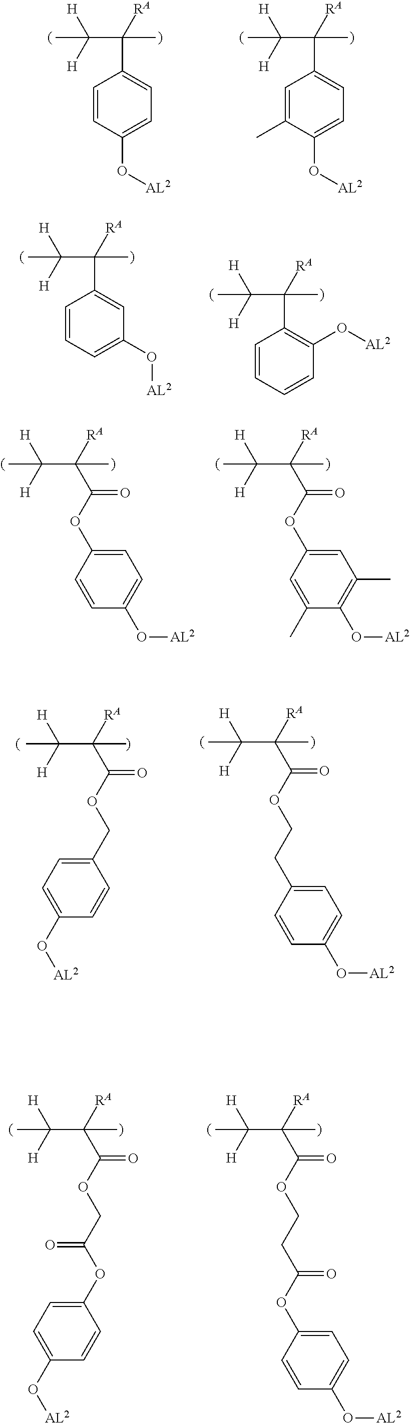

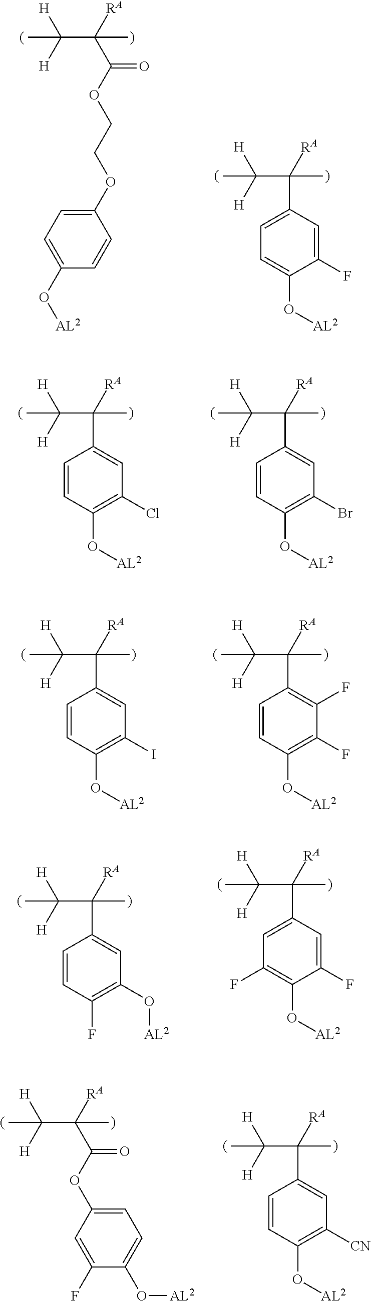

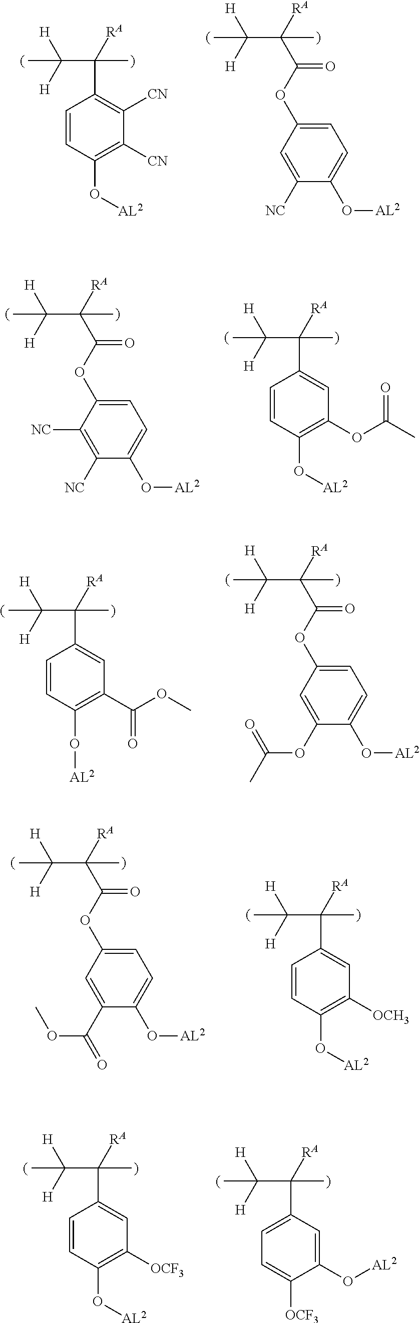

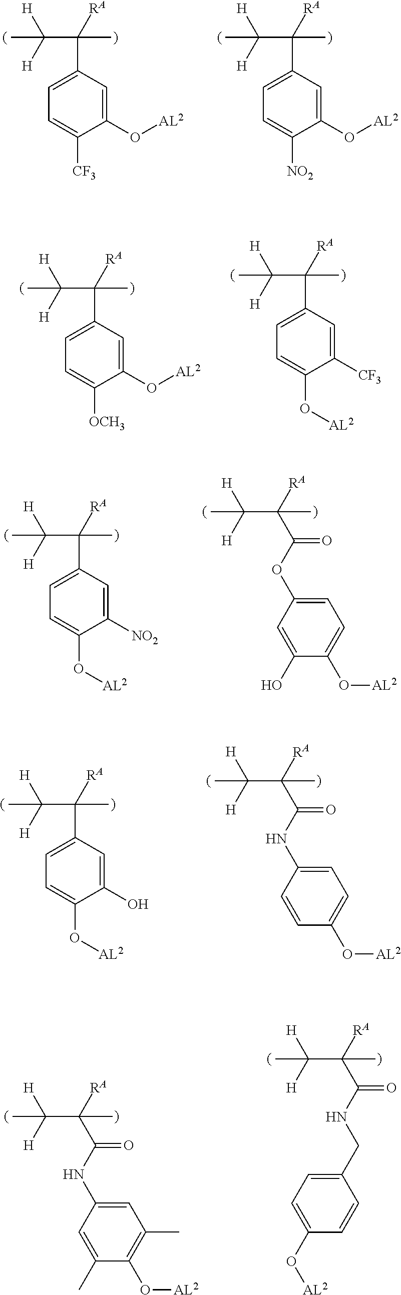

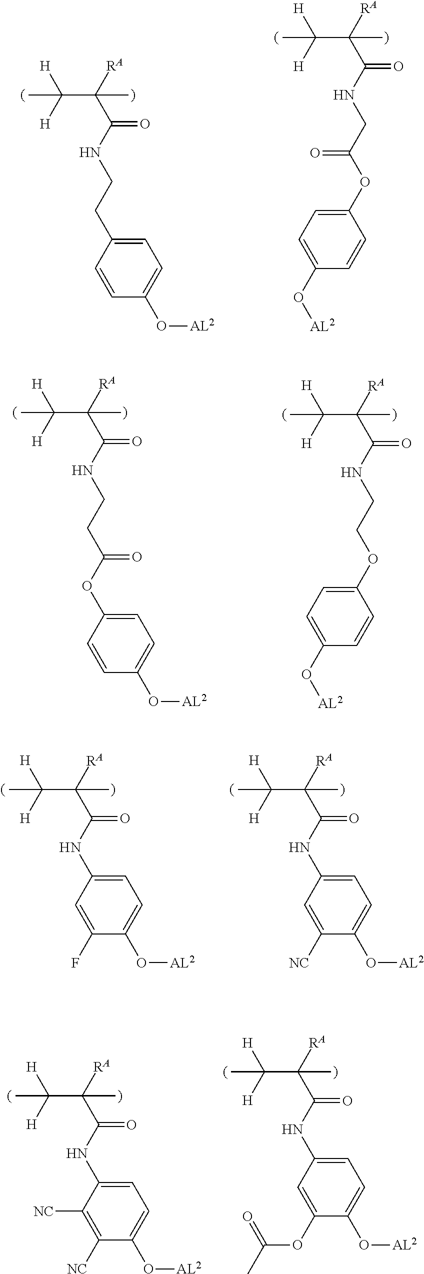

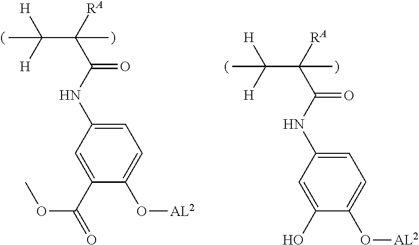

The polymer may further contain a repeat unit having the following formula (b3) (hereinafter, also referred to as a repeat unit b3).

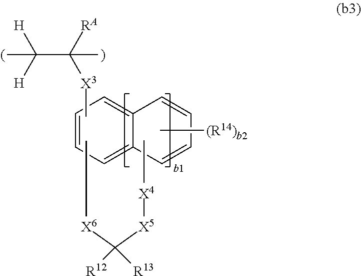

In formula (b3), b1 is 0 or 1. When b1 is 0, it is a benzene ring, and when b1 is 1, it is a naphthalene ring, but a benzene ring in which b1 is 0 is preferable from a viewpoint of solvent solubility. When b1 is 0, b2 is 0, 1, 2, or 3, and when b1 is 1, b2 is 0, 1, 2, 3, 4, or 5. b2 is preferably 0, 1, 2, or 3, and more preferably 0, 1, or 2 from a viewpoint of raw material procurement.

In formula (b3), R^{A} is a hydrogen atom, a fluorine atom, a methyl group, or a trifluoromethyl group. Among these, R^{A} is preferably a hydrogen atom or a methyl group, and more preferably a hydrogen atom.

In formula (b3), X³ is a single bond, *—C(═O)—O—, or *—C(═O)—NH—. * designates a point of attachment to the carbon atom of the main chain. Among these, X³ is preferably a single bond or *—C(═O)—O—, and more preferably a single bond.

In formula (b3), R¹² and R¹³ are each independently a hydrogen atom or a C₁-C₂₀ hydrocarbyl group which may contain a heteroatom. The hydrocarbyl group may be saturated or unsaturated, and may be linear, branched, or cyclic. Specific examples thereof include: a C₁-C₂₀ alkyl group such as a methyl group, an ethyl group, a n-propyl group, an isopropyl group, a n-butyl group, an isobutyl group, a sec-butyl group, a tert-butyl group, a n-pentyl group, a n-hexyl group, a n-octyl group, a n-nonyl group, a n-decyl group, an undecyl group, a dodecyl group, a tridecyl group, a tetradecyl group, a pentadecyl group, a heptadecyl group, an octadecyl group, a nonadecyl group, or an icosyl group; a C₃-C₂₀ cyclic saturated hydrocarbyl group such as a cyclopropyl group, a cyclopentyl group, a cyclohexyl group, a cyclopropylmethyl group, a 4-methylcyclohexyl group, a cyclohexylmethyl group, a norbornyl group, or an adamantyl group; a C₂-C₂₀ alkenyl group such as a vinyl group, an allyl group, a propenyl group, a butenyl group, or a hexenyl group; a C₃-C₂₀ cyclic unsaturated hydrocarbyl group such as a cyclohexenyl group; a C₆-C₂₀ aryl group such as a phenyl group or a naphthyl group; a C₇-C₂₀ aralkyl group such as a benzyl group, a 1-phenylethyl group, or a 2-phenylethyl group; and a group obtained by combining these. Some or all of hydrogen atoms of the hydrocarbyl group may be replaced with a group containing a heteroatom such as an oxygen atom, a sulfur atom, a nitrogen atom, or a halogen atom, some of (—CH₂—)s in the hydrocarbyl group may be replaced with a group containing a heteroatom such as an oxygen atom, a sulfur atom, or a nitrogen atom, and as a result, the hydrocarbyl group may contain a hydroxy group, a cyano group, a fluorine atom, a chlorine atom, a bromine atom, an iodine atom, a carbonyl group, an ether bond, an ester bond, a sulfonic acid ester bond, a carbonate bond, a lactone ring, a sultone ring, a carboxylic acid anhydride (—C(═O)—O—C(═O)—), a haloalkyl group, or the like.

R¹² and R¹³ may be bonded to each other to form a ring together with a carbon atom to which they are bonded. Specific examples of the ring formed at this time include a cyclopropane ring, a cyclobutane ring, a cyclopentane ring, a cyclohexane ring, a norbornane ring, and an adamantane ring. Some or all of hydrogen atoms of the ring may be replaced with a group containing a heteroatom such as an oxygen atom, a sulfur atom, a nitrogen atom, or a halogen atom, some of (—CH₂—)s in the ring may be replaced with a group containing a heteroatom such as an oxygen atom, a sulfur atom, or a nitrogen atom, and as a result, the ring may contain a hydroxy group, a fluorine atom, a chlorine atom, a bromine atom, an iodine atom, a cyano group, a carbonyl group, an ether bond, an ester bond, a sulfonic acid ester bond, a carbonate bond, a lactone ring, a sultone ring, a carboxylic acid anhydride (—C(═O)—O—C(═O)—), a haloalkyl group, or the like.

In formula (b3), R¹⁴ is a halogen atom, a hydroxy group, a cyano group, a nitro group, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{14A})(R^{14B}). R^{14A} and R^{14B} are each independently a hydrogen atom or a C₁-C₆ hydrocarbyl group, The halogen atom is preferably a fluorine atom, a chlorine atom, a bromine atom, or an iodine atom, and more preferably a fluorine atom or an iodine atom. The hydrocarbyl group and hydrocarbyl moieties of the hydrocarbyloxy group, the hydrocarbyloxycarbonyl group, and the hydrocarbylthio group may be saturated or unsaturated, and may be linear, branched, or cyclic. Specific examples thereof include those similar to those exemplified as the hydrocarbyl group of R¹² and R¹³. Some or all of hydrogen atoms of the hydrocarbyl group may be replaced with a group containing a heteroatom such as an oxygen atom, a sulfur atom, a nitrogen atom, or a halogen atom, some of (—CH₂—)s in the hydrocarbyl group may be replaced with a group containing a heteroatom such as an oxygen atom, a sulfur atom, or a nitrogen atom, and as a result, the hydrocarbyl group may contain a hydroxy group, a cyano group, a fluorine atom, a chlorine atom, a bromine atom, an iodine atom, a carbonyl group, an ether bond, an ester bond, a sulfonic acid ester bond, a carbonate bond, a lactone ring, a sultone ring, a carboxylic acid anhydride (—C(═O)—O—C(═O)—), a haloalkyl group, or the like. When b2 is 2 or more, R¹⁴s may be the same as or different from each other.

When b2 is 2 or more, a plurality of R¹⁴s may be bonded to each other to form a ring together with aromatic ring carbon atoms to which they are bonded. Specific examples of the ring formed at this time include a cyclopropane ring, a cyclobutane ring, a cyclopentane ring, a cyclohexane ring, a norbornane ring, and an adamantane ring. Some or all of hydrogen atoms of the ring may be replaced with a group containing a heteroatom such as an oxygen atom, a sulfur atom, a nitrogen atom, or a halogen atom, some of (—CH₂—)s in the ring may be replaced with a group containing a heteroatom such as an oxygen atom, a sulfur atom, or a nitrogen atom, and as a result, the ring may contain a hydroxy group, a fluorine atom, a chlorine atom, a bromine atom, an iodine atom, a cyano group, a carbonyl group, an ether bond, an ester bond, a sulfonic acid ester bond, a carbonate bond, a lactone ring, a sultone ring, a carboxylic acid anhydride (—C(═O)—O—C(═O)—), a haloalkyl group, or the like.

In formula (b3), X⁴ is a single bond, a C₁-C₄ aliphatic hydrocarbylene group, a carbonyl group, a sulfonyl group, or a group obtained by combining these groups. Among these, X⁴ is preferably a single bond, a carbonyl group, or a sulfonyl group from a viewpoint of raw material procurement, and is more preferably a single bond or a carbonyl group from a viewpoint of a polar group generated after reaction.

In formula (b3), X⁵ and X⁶ are each independently an oxygen atom or a sulfur atom. Provided that X⁴ and X⁶ are bonded to adjacent aromatic ring carbon atoms. X⁵ and X⁶ may be the same as or different from each other, but X⁵ and X⁶ are both preferably oxygen atoms from a viewpoint of reactivity.

Specific examples of the repeat unit b3 include, but are not limited to, those listed below. Note that, in the following formulas, R^{A} is as defined above, and Me is a methyl group. Bonding positions of various substituents on an aromatic ring may be replaced with each other.

1. An onium salt monomer having the following formula (a): 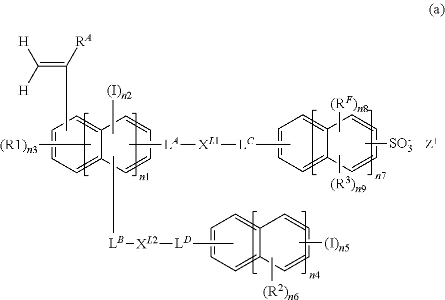
wherein n1 is 0 or 1, n2 is 1, 2, 3, or 4, n3 is 0, 1, or 2, provided that 1≤n2+n3≤4 when n1 is 0, and 1≤n2+n3≤6 when n1 is 1, n4 is 0 or 1, n5 is 1, 2, 3, or 4, n6 is 0, 1, or 2, provided that 1≤n5+n6≤4 when n4 is 0, and 1≤n5+n6≤6 when n4 is 1, n7 is 0 or 1, n8 is 0, 1, 2, 3, or 4, and n9 is 0, 1, or 2, provided that 0≤n8+n9≤4 when n7 is 0, and 0≤n8+n9≤6 when n7 is 1,
R^{A} is a hydrogen atom, a fluorine atom, a methyl group, or a trifluoromethyl group,
R¹ is a halogen atom other than an iodine atom, a nitro group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when n3 is 2, R's may be the same as or different from each other, and two R's may be bonded to each other to form a ring together with carbon atoms to which they are bonded,
R² is a halogen atom other than an iodine atom, a nitro group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when n6 is 2, R²s may be the same as or different from each other, and two R²s may be bonded to each other to form a ring together with carbon atoms to which they are bonded,
R³ is a halogen atom other than a fluorine atom, a nitro group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when n9 is 2, R³s may be the same as or different from each other, and two R³s may be bonded to each other to form a ring together with carbon atoms to which they are bonded,
R^{F} is a fluorine atom, a C₁-C₆ fluorinated saturated hydrocarbyl group, a C₁-C₆ fluorinated saturated hydrocarbyloxy group, or a C₁-C₆ fluorinated saturated hydrocarbylthio group, when n8 is 2, 3, or 4, R's may be the same as or different from each other,
L^{A}, L^{B}, L^{C}, and LP are each independently a single bond, an ether bond, an ester bond, a sulfonic acid ester bond, an amide bond, a sulfonic acid amide bond, a carbonate bond, or a carbamate bond,
X^{L1} and X^{L2} are each independently a single bond or a C₁-C₄₀ hydrocarbylene group which may contain a heteroatom, and
Z⁺ is an onium cation.

2. The onium salt monomer according to claim 1, which has the following formula (a1): 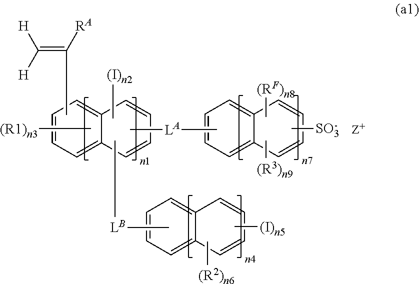
wherein n1 to n9, R^{A}, R¹, R², R³, R^{F}, L^{A}, L^{B}, and Z⁺ are as defined above.

3. The onium salt monomer according to claim 2, which has the following formula (a2): 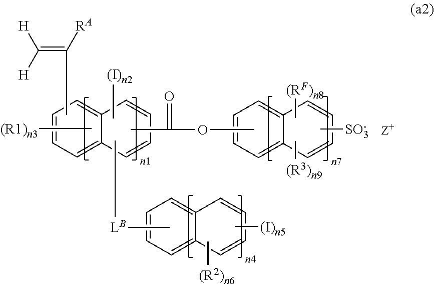
wherein n1 to n9, R^{A}, R¹, R², R³, R^{F}, L^{B}, and Z⁺ are as defined above.

4. The onium salt monomer according to claim 1, wherein Z⁺ is a sulfonium cation having the following formula (Z-1) or an iodonium cation having the following formula (Z-2): 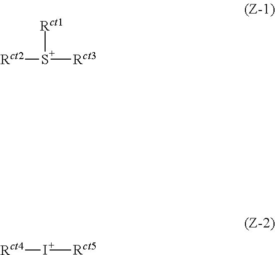
wherein R^{ct1} to R^{ct5} are each independently a halogen atom or a C₁-C₃₀ hydrocarbyl group which may contain a heteroatom, and R^{ct1} and R^{ct2} may be bonded to each other to form a ring together with a sulfur atom to which they are bonded.

5. The onium salt monomer according to claim 1, wherein Z⁺ is a sulfonium cation having the following formula (Z-3): 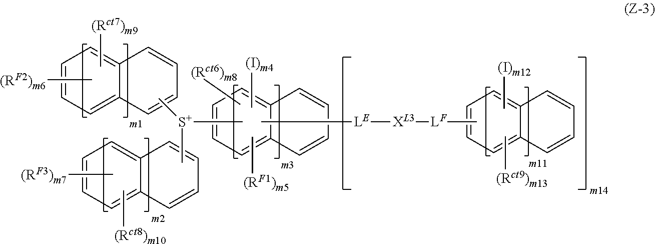
wherein m1 is 0 or 1, m2 is 0 or 1, m3 is 0 or 1, m4 is 0, 1, 2, 3, or 4, m5 is 0, 1, 2, 3, or 4, m6 is 0, 1, 2, 3, 4, 5, or 6, m7 is 0, 1, 2, 3, 4, 5, or 6, m8 is 0, 1, or 2, m9 is 0, 1, or 2, m10 is 0, 1, or 2, m11 is 0 or 1, m12 is 0, 1, 2, 3, or 4, m13 is 0, 1, or 2, m14 is 0, 1, or 2, provided that 0≤m6+m9≤4 when m1 is 0, and 0≤m6+m9≤6 when m1 is 1, 0≤m7+m10≤4 when m2 is 0, and 0≤m7+m10≤6 when m2 is 1, 1≤m4+m5+m8+m14≤4 when m3 is 0, and 1≤m4+m5+m8+m14≤6 when m3 is 1, 0≤m12+m13≤4 when m11 is 0, and 0≤m12+m13≤6 when m11 is 1, and m4+m12≥1,
R^{F1} to R^{F3} are each independently a fluorine atom, a C₁-C₆ fluorinated saturated hydrocarbyl group, a C₁-C₆ fluorinated saturated hydrocarbyloxy group, or a C₁-C₆ fluorinated saturated hydrocarbylthio group, when m5 is 2 or more, R^{F1}s may be the same as or different from each other, when m6 is 2 or more, R^{F2}s may be the same as or different from each other, and when m7 is 2 or more, R^{F3}s may be the same as or different from each other,
each of R^{ct6} to R^{ct9} is a halogen atom other than an iodine atom and a fluorine atom, a nitro group, a cyano group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when m8 is 2, two R^{ct6}s may be the same or different from each other, and may be bonded to each other to form a ring together with carbon atoms to which they are bonded, when m9 is 2, two R^{ct7}s may be the same or different from each other, and may be bonded to each other to form a ring together with carbon atoms to which they are bonded, when m10 is 2, two R^{ct8}s may be the same or different from each other, and may be bonded to each other to form a ring together with carbon atoms to which they are bonded, when m13 is 2, two R^{ct9}s may be the same or different from each other, and may be bonded to each other to form a ring with carbon atoms to which they are bonded,
aromatic rings directly bonded to S⁺ in the sulfonium cation may be bonded to each other to form a ring together with S⁺,
L^{E} and L^{F} are each independently a single bond, an ether bond, an ester bond, an amide bond, a sulfonic acid ester bond, a sulfonic acid amide bond, a carbonate bond, or a carbamate bond, and
X^{L3} is a single bond or a C₁-C₄₀ hydrocarbylene group which may contain a heteroatom.

6. The onium salt monomer according to claim 5, wherein the sulfonium cation having formula (Z-3) has the following formula (Z-3-1): 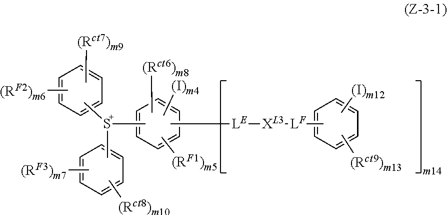
wherein m4 to m10, m12 to m14, R^{F1} to R^{F3}, Rct6 to R^{ct9}, L^{E}, L^{F}, and X¹³ are as defined above.

7. The onium salt monomer according to claim 6, wherein the sulfonium cation having formula (Z-3-1) has the following formula (Z-3-2): 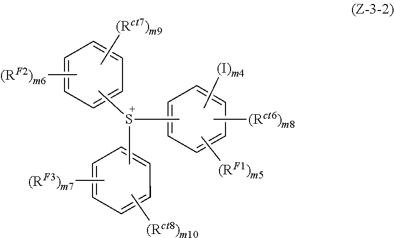
wherein m4 to m10, R^{F1} to R^{F3}, and R^{ct6} to R^{ct8} are as defined above.

8. A monomeric photoacid generator comprising the onium salt monomer according to claim 1.

9. A polymer comprising a repeat unit derived from the monomeric photoacid generator according to claim 8.

10. The polymer according to claim 9, further comprising a repeat unit having the following formula (b1) or (b2): 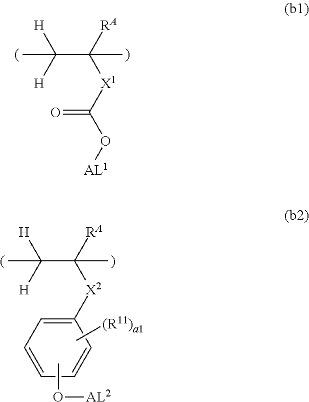
wherein R^{A}s are each independently a hydrogen atom, a fluorine atom, a methyl group, or a trifluoromethyl group,
X¹ is a single bond, a phenylene group, a naphthylene group, *—C(═O)—O—X¹¹—, or *—C(═O)—NH—X¹¹—, the phenylene group or the naphthylene group may be substituted with a hydroxy group, a nitro group, a cyano group, a C₁-C₁₀ saturated hydrocarbyl group which may contain a fluorine atom, a C₁-C₁₀ saturated hydrocarbyloxy group which may contain a fluorine atom, or a halogen atom, X¹¹ is a C₁-C₁₀ saturated hydrocarbylene group, a phenylene group, or a naphthylene group, and the saturated hydrocarbylene group may contain a hydroxy group, an ether bond, an ester bond, or a lactone ring,
X² is a single bond, *—C(═O)—O—, or *—C(═O)—NH—,
* designates a point of attachment to the carbon atom of the main chain,
R¹¹ is a halogen atom, a cyano group, a hydroxy group, a nitro group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom,
AL¹ and AL² are each independently an acid-unstable group, and
a1 is 0, 1, 2, 3, or 4.

11. The polymer according to claim 9, further comprising a repeat unit having the following formula (b3): 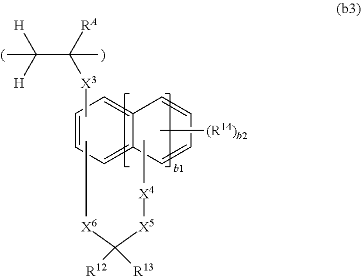
wherein b1 is 0 or 1, b2 is 0, 1, 2, or 3 when b1 is 0, and b2 is 0, 1, 2, 3, 4, or 5 when b1 is 1,
R^{A} is a hydrogen atom, a fluorine atom, a methyl group, or a trifluoromethyl group,
X³ is a single bond, *—C(═O)—O—, or *—C(═O)—NH—, * designates a point of attachment to the carbon atom of the main chain,
R¹² and R¹³ are each independently a hydrogen atom or a C₁-C₂₀ hydrocarbyl group which may contain a heteroatom, R¹² and R¹³ may be bonded to each other to form a ring together with a carbon atom to which they are bonded,
R¹⁴ is a halogen atom, a hydroxy group, a cyano group, a nitro group, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{14A})(R^{14B}), R^{14A} and R^{14B} are each independently a hydrogen atom or a C₁-C₆ hydrocarbyl group, when b2 is 2 or more, a plurality of R¹⁴s may be bonded to each other to form a ring together with aromatic ring carbon atoms to which they are bonded,
X⁴ is a single bond, a C₁-C₄ aliphatic hydrocarbylene group, a carbonyl group, a sulfonyl group, or a group obtained by combining these groups, and
X⁵ and X⁶ are each independently an oxygen atom or a sulfur atom, provided that X⁴ and X⁶ are bonded to adjacent aromatic ring carbon atoms.

12. The polymer according to any one of claim 9, further comprising a repeat unit having the following formula (c): 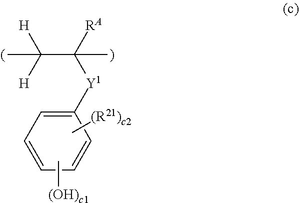
wherein R^{A} is a hydrogen atom, a fluorine atom, a methyl group, or a trifluoromethyl group,
Y¹ is a single bond, *—C(═O)—O—, or *—C(═O)—NH—, * designates a point of attachment to the carbon atom of the main chain,
R²¹ is a halogen atom, a nitro group, a cyano group, a carboxy group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom,
c1 is 1, 2, 3, or 4, and c2 is 0, 1, 2, or 3, provided that 1≤c1+c2≤5.

13. The polymer according to claim 9, further comprising a repeat unit having the following formula (d): 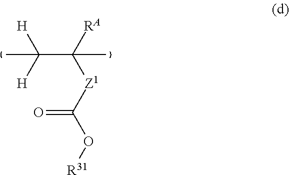
wherein R^{A} is a hydrogen atom, a fluorine atom, a methyl group, or a trifluoromethyl group,
Z¹ is a single bond, a phenylene group, a naphthylene group, *—C(═O)—O—Z¹¹—, or *—C(═O)—NH—Z¹¹—, the phenylene group or the naphthylene group may be substituted with a hydroxy group, a nitro group, a cyano group, a C₁-C₁₀ saturated hydrocarbyl group which may contain a fluorine atom, a C₁-C₁₀ saturated hydrocarbyloxy group which may contain a fluorine atom, or a halogen atom, * designates a point of attachment to the carbon atom of the main chain, Z¹¹ is a C₁-C₁₀ saturated hydrocarbylene group, a phenylene group, or a naphthylene group, and the saturated hydrocarbylene group may contain a hydroxy group, an ether bond, an ester bond, or a lactone ring, and
R³¹ is a hydrogen atom or a C₁-C₂₀ group containing at least one structure selected from a hydroxy group other than a phenolic hydroxy group, a cyano group, a carbonyl group, a carboxy group, an ether bond, an ester bond, a sulfonic acid ester bond, a carbonate bond, a lactone ring, a sultone ring, and a carboxylic anhydride (—C(═O)—O—C(═O)—).

14. A chemically amplified resist composition comprising a base polymer including (A) the polymer according to claim 9.

15. The chemically amplified resist composition according to claim 14, further comprising (B) an organic solvent.

16. The chemically amplified resist composition according to claim 14, further comprising (C) a quencher.

17. The chemically amplified resist composition according to claim 14, further comprising (D) an acid generator.

18. The chemically amplified resist composition according to claim 14, further comprising (E) a surfactant.

19. A pattern forming method comprising: a step of forming a resist film on a substrate using the chemically amplified resist composition according to claim 14; a step of exposing the resist film to a high energy ray; and a step of developing the exposed resist film using a developer.

20. The pattern forming method according to claim 19, wherein the high energy ray is ArF excimer laser light having a wavelength of 193 nm, KrF excimer laser light having a wavelength of 248 nm, an electron beam, or an extreme ultraviolet ray having a wavelength of 3 to 15 nm.
